(19) United States
(12) Patent Application Publication
(10) Pub. No.: US 2026/0217713 A1
(43) Pub. Date: Jul. 30, 2026

(54) KAT6 TARGETING COMPOUNDS

(71) Applicant: Prelude Therapeutics Incorporated, Wilmington, DE (US)

(72) Inventors: Andrew Buesking, Wilmington, DE (US); Sarah Pawley, Landenberg, PA (US); Chun Chen, Claymont, DE (US); Vijay Devannah, Newark, DE (US); Sina Rezazadeh, Newark, DE (US); Corey Basch, West Chester, PA (US)

(21) Appl. No.: 19/426,760

(22) Filed: Dec. 19, 2025

Related U.S. Application Data
(60) Provisional application No. 63/736,960, filed on Dec. 20, 2024.
(60) Provisional application No. 63/822,904, filed on Jun. 13, 2025.
(60) Provisional application No. 63/922,437, filed on Nov. 21, 2025.

(51) Int. Cl. C07D 471/10; A61K 31/454; A61K 31/4545; A61K 31/4725; A61K 31/496; A61K 31/4985; A61K 31/506; A61K 31/513; C07B 59/00; C07D 413/14; C07D 451/02; C07D 471/04; C07D 487/04; C07D 519/00

(52) U.S. Cl. CPC C07D 471/10; A61K 31/454; A61K 31/4545; A61K 31/4725; A61K 31/496; A61K 31/4985; A61K 31/506; A61K 31/513; C07B 59/002; C07D 413/14; C07D 451/02; C07D 471/04; C07D 487/04; C07D 519/00; C07B 2200/05

(57) ABSTRACT

The present disclosure provides bifunctional compounds comprising a target protein binding moiety and a E3 ubiquitin ligase binding moiety, and associated methods of use.

### CROSS REFERENCE TO RELATED APPLICATIONS

This application claims the benefit of U.S. Provisional Application Nos. 63/736,960 filed Dec. 20, 2024, 63/822,904 filed Jun. 13, 2025, and 63/922,437 filed Nov. 21, 2025, the entireties of which are incorporated by reference herein.

### TECHNICAL FIELD

The description provides bifunctional compounds comprising a target protein binding moiety and a E3 ubiquitin ligase binding moiety, and associated methods of use. The bifunctional compounds are useful as modulators of targeted KAT6 proteins, which are degraded and/or otherwise inhibited by bifunctional compounds according to the present disclosure.

### BACKGROUND OF THE INVENTION

Epigenetic control of transcription via chromatin remodeling is a substantial regulatory mechanism that ultimately impacts the functional expression of many proteins. Aberrations to epigenetic regulators are contributors to human diseases and are considered major oncogenic drivers in many cancers. Regulation of histones plays an essential role in this process and epigenetic regulators modify histone proteins through assorted post-translational modifications such as acetylation and methylation to alter, for example, chromatin accessibility. Histone acetylation is catalyzed by histone acetyltransferases (HATs), and dysregulation of this class of epigenetic regulators is linked to transcriptional alterations and cancer (1, 2).

The MYST family of acetyltransferases have been linked to several types of cancer and contain a conserved catalytic domain (MYST domain). Lysine acetyltransferase 6a (KAT6A, MOZ, MYST3) is a member of this family, promotes histone acetylation, and is paralogous to lysine acetyltransferase 6b (KAT6B) (3, 4). KAT6A reportedly acetylates lysine 9 (H3K9ac), lysine 14 (H3K14ac)(4), and lysine 23 (H3K23ac) (5) of histone H3, although the functional dependency on these sites, and other potential substrates, remains poorly understood. In addition to histone proteins, KAT6A reportedly acetylates non-histone proteins such as p53 (6), but its role in regulating non-histone substrates is not clear. KAT6A can form a protein complex with inhibitor of growth family member 5 (ING5), bromodomain and PHD finger containing 1, 2 or 3 (BRPF1, BRPF2, BRPF3), and myst/Esa1 associated factor 6 (EAF6), which may enhance its gene regulatory capacity (4, 7). Mechanistically, KAT6A is linked to regulation of cellular processes such as senescence, cell cycle progression, and hematopoietic stem cell maintenance (4, 7, 8).

More recently, KAT6A was shown to epigenetically regulate estrogen receptor alpha (ER alpha) expression via its HAT domain in KAT6A amplified breast cancer cell lines. Loss of KAT6A was associated with reduced proliferation in vitro and tumor growth in vivo and its overexpression correlates with worse clinical outcome in estrogen receptor positive (ER+) breast cancers (9). Inhibitors of KAT6A reportedly show greater sensitivity in the ER+ luminal subtype (ER/PR+/HER2−) (10). KAT6A is suggested to be an oncogenic in breast, brain, hematological, gynecological, and other cancers (5, 9, 11, 12).

KAT6A is frequently altered in many types of cancers, most often by copy number amplification or recurrent chromosomal translocations. Located within the recurrently amplified chromosomal 8p11-12 amplicon, KAT6A amplification is observed in numerous cancers including breast, lung, prostate, blood, bladder, uterine, endometrial, ovarian, esophageal, head and neck, stomach, colon, and other cancers (9, 13, 14). Especially in hematological cancers, recurrent rearrangements lead to several fusion proteins like KAT6A-CBP, KAT6A-p300, KAT6A-TIF2, KAT6A-NcoA3, and KAT6A-LEUTX (7). Recently, KAT6A has been implicated as important for the growth of MLL-rearranged AML (11).

To date, only one KAT6A targeted small molecule inhibitor is currently in clinical trials (PF-07248144, Phase 1, Pfizer) to study effects in locally advanced or metastatic ER+/HER2− breast, castration-resistant prostate, and non-small cell lung cancer as a single agent or in combination with either fulvestrant or letrozole+palbociclib (NCT04606446) (15). It remains unclear whether traditional small molecule inhibitors of KAT6A can unlock the full potential of targeting these or other cancers in a selective manner. Therefore, exploring a targeted protein degradation (TPD) approach to develop potent and selective KAT6A degraders could enhance the therapeutic window in cancers dependent on KAT6A for growth, proliferation, or survival.

In light of the established role of KATs in diseases such as cancer, a need exists for new modulators of these proteins.

### REFERENCES

1. Nair S S, Kumar R. Chromatin remodeling in cancer: a gateway to regulate gene transcription. Mol Oncol. 2012; 6(6):611-9.
2. Farria A, Li W, Dent S Y. KATs in cancer: functions and therapies. Oncogene. 2015; 34(38):4901-13.
3. Avvakumov N, Côté J. The MYST family of histone acetyltransferases and their intimate links to cancer. Oncogene. 2007; 26(37):5395-407.
4. Su J, Wang X, Bai Y, Sun M, Yao Y, Duan Y. The role of MOZ/KAT6A in hematological malignancies and advances in MOZ/KAT6A inhibitors. Pharmacol Res. 2021; 174:105930.
5. Lv D, Jia F, Hou Y, Sang Y, Alvarez A A, Zhang W, et al. Histone Acetyltransferase KAT6A Upregulates PI3K/AKT Signaling through TRIM24 Binding. Cancer Res. 2017; 77(22):6190-201.
6. Rokudai S, Laptenko O, Arnal S M, Taya Y, Kitabayashi I, Prives C. MOZ increases p53 acetylation and premature senescence through its complex formation with PML. Proc Natl Acad Sci USA. 2013; 110(10):3895-900.
7. Huang F, Abmayr S M, Workman J L. Regulation of KAT6 Acetyltransferases and Their Roles in Cell Cycle Progression, Stem Cell Maintenance, and Human Disease. Mol Cell Biol. 2016; 36(14):1900-7.
8. Sheikh B N, Phipson B, El-Saafin F, Vanyai H K, Downer N L, Bird M J, et al. MOZ (MYST3, KAT6A) inhibits senescence via the INK4A-ARF pathway. Oncogene. 2015; 34(47):5807-20.
9. Yu L, Liang Y, Cao X, Wang X, Gao H, Lin S Y, et al. Identification of MYST3 as a novel epigenetic activator of ERα frequently amplified in breast cancer. Oncogene. 2017; 36(20):2910-8.
10. Sharma S, Chung J, Uryu S, Rickard A, Nady N, Khan S, et al. Abstract 1130: First-in-class KAT6A/KAT6B inhibitor CTx-648 (PF-9363) demonstrates potent anti-tumor activity in ER+ breast cancer with KAT6A dysregulation. Cancer Research. 2021; 81(13_Supplement):1130-.
11. Yan F, Li J, Milosevic J, Petroni R, Liu S, Shi Z, et al. KAT6A and ENL Form an Epigenetic Transcriptional Control Module to Drive Critical Leukemogenic Gene-Expression Programs. Cancer Discov. 2022; 12(3):792-811.
12. Liu W, Zhan Z, Zhang M, Sun B, Shi Q, Luo F, et al. KAT6A, a novel regulator of 3-catenin, promotes tumorigenicity and chemoresistance in ovarian cancer by acetylating COP1. Theranostics. 2021; 11(13):6278-92.
13. Saglam O, Tang Z, Tang G, Medeiros L J, Toruner G A. KAT6A amplifications are associated with shorter progression-free survival and overall survival in patients with endometrial serous carcinoma. PLoS One. 2020; 15(9):e0238477.
14. Cerami E, Gao J, Dogrusoz U, Gross B E, Sumer S O, Aksoy B A, et al. The cBio cancer genomics portal: an open platform for exploring multidimensional cancer genomics data. Cancer Discov. 2012; 2(5):401-4.
15. ClinicalTrials.gov. NCT04606446: Study of PF-07248144 in Advanced or Metastatic Solid Tumors (KAT6) Bethesda (M D): National Library of Medicine (U S); 2020 [Available from: https://clinicaltrials.gov/ct2/show/NCT04606446.

### SUMMARY OF THE INVENTION

The present disclosure is directed to compounds of Formula (I):

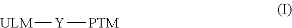

or a pharmaceutically acceptable salt or solvate thereof,
wherein Y-PTM is a moiety of Formula IA:

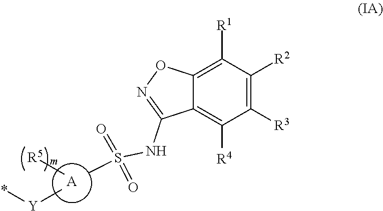

wherein
   Y is a covalent bond, or chemical moiety that links PTM and ULM;
   * is a point of attachment to ULM;
   Ring A is a C₆-C₁₀ membered aryl group or a 5-10 membered heteroaryl group;
   R¹ is H or 5-6 membered heteroaryl optionally substituted by methyl;
   R² is H, C₁₋₄alk-O—C₁₋₄alkyl, or —(C(R⁸)₂)ₙ-(5-9 membered heteroaryl) optionally substituted by halogen, C₁-C₃ alkyl, —CH₂OH, or —OH;
   R³ is H, halogen, C₁-C₄ alkyl, cyclopropyl, haloalkyl, C₁-C₄ alkoxy, or haloalkoxy;
   R⁴ is H, halogen, C₁-C₄ alkyl, cyclopropyl, C₁-C₄ alkoxy, or —O-cyclopropyl;
   each R⁵ is independently H, halogen, oxo, —OH, —CN, —NO₂, —C₁-C₆alkyl, —C₂-C₆alkenyl, —C₂-C₆alkynyl, C₀-C₁alk-aryl, C₀-C₁alk-heteroaryl, cycloalkyl, cycloalkenyl, heterocycloalkyl, or heterocycloalkenyl, —OR^{a}, —SR^{a}, —NR^{c}R^{d}, —NR^{a}R^{c}, —C(O)R^{b}, —OC(O)R^{a}, —C(O)OR^{a}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{d})(OR^{c}), or —S(O)₂R^{b};
   each R^{a} is independently H, —C(O)R^{b}, —C(O)OR^{c}, —C(O)NR^{c}R^{d}, —C(═NR^{b})NR^{b}R^{c}, —C(═NOR^{b})R^{b}R^{c}, —C(═NCN)NR^{b}R^{c}, —P(OR^{c})₂, —P(O)R^{c}R^{b}, —P(O)OR^{c}OR^{b}, —S(O)R^{b}, —S(O)NR^{c}R^{d}, —S(O)₂R^{b}, —S(O)₂NR^{c}R^{d}, SiR^{b}₃, —C₁-C₁₀alkyl, —C₁-C₁₀ haloalkyl, —C₂-C₁₀ alkenyl, —C₂-C₁₀ alkynyl, aryl, cycloalkyl, cycloalkenyl, heteroaryl, heterocycloalkyl, or heterocycloalkenyl;
   each R^{b} is independently H, —C₁-C₆ alkyl, —C₁-C₆ haloalkyl, —C₂-C₆ alkenyl, —C₂-C₆ alkynyl, aryl, cycloalkyl, cycloalkenyl, heteroaryl, heterocycloalkyl, or heterocycloalkenyl;
   each R^{c} or R^{d} is independently H, —C₁-C₁₀ alkyl, —C₂-C₆ alkenyl, —C₂-C₆ alkynyl, —OC₁-C₆alkyl, —O-cycloalkyl, aryl, heteroaryl, cycloalkyl, cycloalkenyl, heterocycloalkyl, or heterocycloalkenyl; or
   R^{c} and R^{d}, together with the atom to which they are both attached, form a monocyclic or multicyclic heterocycloalkyl, or a monocyclic or multicyclic heterocyclo-alkenyl group;
   each R⁸ is independently H, halogen, or C₁-C₄ alkyl;
   m is 0, 1, 2, 3, or 4;
   n is 0 or 1; and
   ULM is

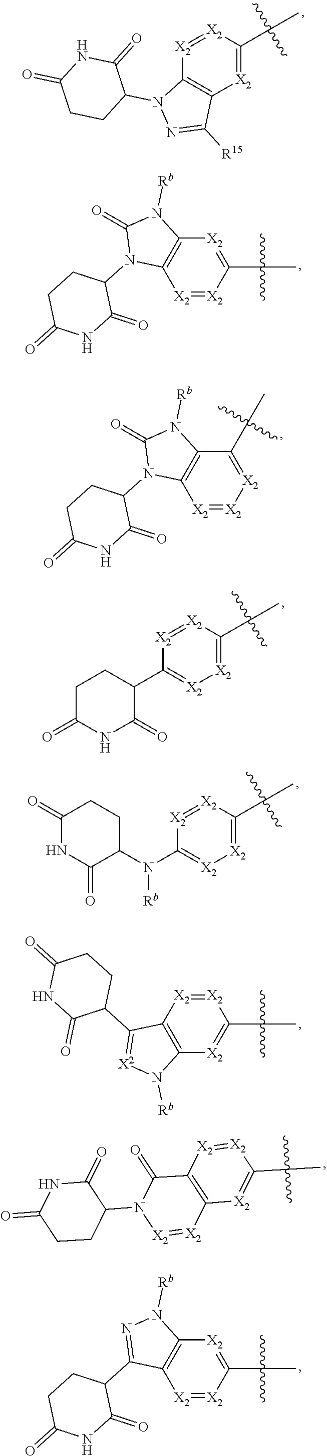

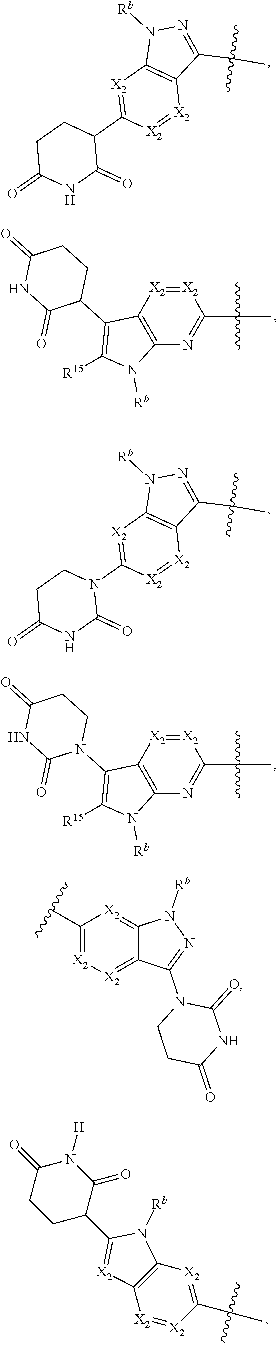

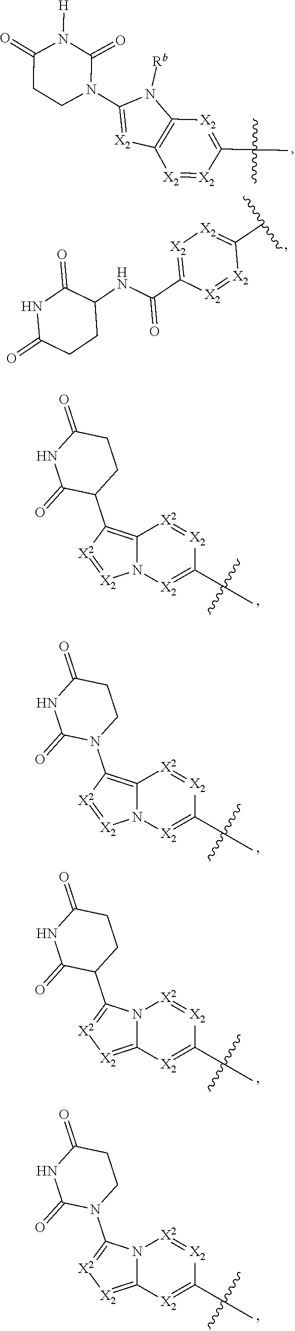

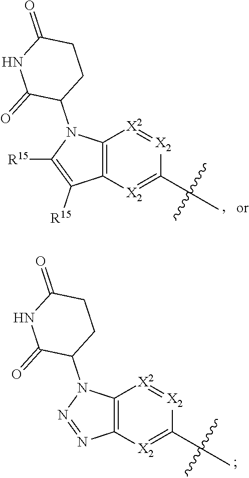

wherein
[symbol] is a point of attachment to Y;
each X₂ is independently N or CR¹⁵; and
each R¹⁵ is independently H, D, halogen, oxo, —OH, —CN, —NO₂, —C₁-C₆alkyl, —C₁-C₆ haloalkyl, —C₂-C₆alkenyl, —C₂-C₆alkynyl, C₀-C₁alk-aryl, C₀-C₁alk-heteroaryl, cycloalkyl, cycloalkenyl, heterocycloalkyl, or heterocycloalkenyl, —OR^{a}, —SR^{a}, —NR^{c}R^{d}, —NR^{a}R^{c}, —C(O)R^{b}, —OC(O)R^{a}, —C(O)OR^{a}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{d})(OR^{c}) or —S(O)₂R^{b};
wherein any of the —C₁-C₆alkyl, C₁-C₈ alkoxide, —C₂-C₆alkenyl, —C₂-C₆alkynyl, aryl, heteroaryl, cycloalkyl, cycloalkenyl, heterocycloalkyl, heterocycloalkenyl, spirocycloalkyl, spiroheterocycloalkyl and spiroheterocycloalkenyl groups in any of R₁-R₅, R₈, R₁₅, and ring A is optionally substituted by 1-6 R^{f} groups; and
each R^{f} is independently D, oxo, halogen, C₁-C₈ alkoxy, C₁-C₈ alkyl, C₁₋₆ haloalkyl, C₁₋₆ haloalkoxy, —OH, —CN, —NO₂, —C₂₋₆ alkenyl, —C₂₋₆ alkynyl, C₆₋₁₀ aryl, C₅₋₁₂ heteroaryl, C₃₋₈ cycloalkyl, C₃₋₈ cycloalkenyl, C₃₋₈ heterocycloalkyl, C₃₋₈ heterocycloalkenyl, —OR^{a}, —SR^{a}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)R^{c}R^{d}, —P(O)(OR^{b})(OR^{b}), —B(OR^{c})(OR^{d}), —S(O)₂R^{b}, —C(O)NR^{b}OR^{b}, —S(O)₂OR^{b}, —OS(O)₂OR^{b}, or —OPO(OR^{b})(OR^{b}); wherein said C₁-C₈ alkyl is optionally substituted by 1-6 groups selected from D, halogen, —OH, —CN, —OR^{a}, —SR^{a}, —NR^{a}R^{d}, or NR^{c}R^{d}.

Stereoisomers of the compounds of Formula I, and the pharmaceutical salts and stereoisomers thereof, are also contemplated, described, and encompassed herein. Methods of using compounds of Formula I are described, as well as pharmaceutical compositions including the compounds of Formula I.

### DETAILED DESCRIPTION OF ILLUSTRATIVE EMBODIMENTS

Unless otherwise defined, all technical and scientific terms used herein have the same meaning as commonly understood by one of ordinary skill in the art to which this disclosure pertains. The terminology used in the description is for describing particular embodiments only and is not intended to be limiting of the disclosure.

Where a range of values is provided, it is understood that each intervening value, to the tenth of the unit of the lower limit unless the context clearly dictates otherwise (such as in the case of a group containing a number of carbon atoms in which case each carbon atom number falling within the range is provided), between the upper and lower limit of that range and any other stated or intervening value in that stated range is encompassed within the disclosure. The upper and lower limits of these smaller ranges may independently be included in the smaller ranges is also encompassed within the disclosure, subject to any specifically excluded limit in the stated range. Where the stated range includes one or both of the limits, ranges excluding both of those included limits are also included in the disclosure.

The following terms are used to describe the present disclosure. In instances where a term is not specifically defined herein, that term is given an art-recognized meaning by those of ordinary skill applying that term in context to its use in describing the present disclosure.

The articles “a” and “an” as used herein and in the appended claims are used herein to refer to one or to more than one (e.g., to at least one) of the grammatical object of the article unless the context clearly indicates otherwise. By way of example, “an element” means one element or more than one element.

The terms “co-administration” and “co-administering” or “combination therapy” refer to both concurrent administration (administration of two or more therapeutic agents at the same time) and time varied administration (administration of one or more therapeutic agents at a time different from that of the administration of an additional therapeutic agent or agents), as long as the therapeutic agents are present in the patient to some extent, preferably at effective amounts, at the same time. In certain preferred aspects, one or more of the present compounds described herein, are co-administered in combination with at least one additional bioactive agent, especially including an anticancer agent. In particularly preferred aspects, the co-administration of compounds results in synergistic activity and/or therapy, including anticancer activity.

The term “compound”, as used herein, unless otherwise indicated, refers to any specific chemical compound disclosed herein and includes tautomers, regioisomers, geometric isomers, and where applicable, stereoisomers, including optical isomers (enantiomers) and other stereoisomers (diastereomers) thereof, as well as pharmaceutically acceptable salts and derivatives, including prodrug and/or deuterated forms thereof where applicable, in context. Deuterated small molecules contemplated are those in which one or more of the hydrogen atoms contained in the drug molecule have been replaced by deuterium.

Within its use in context, the term compound generally refers to a single compound, but also may include other compounds such as stereoisomers, regioisomers and/or optical isomers (including racemic mixtures) as well as specific enantiomers or enantiomerically enriched mixtures of disclosed compounds. The term also refers, in context to prodrug forms of compounds which have been modified to facilitate the administration and delivery of compounds to a site of activity. It is noted that in describing the present compounds, numerous substituents and variables associated with same, among others, are described. It is understood by those of ordinary skill that molecules which are described herein are stable compounds as generally described hereunder.

The term “ubiquitin ligase” refers to a family of proteins that facilitate the transfer of ubiquitin to a specific substrate protein, targeting the substrate protein for degradation. For example, an E3 ubiquitin ligase protein that alone or in combination with an E2 ubiquitin-conjugating enzyme causes the attachment of ubiquitin to a lysine on a target protein, and subsequently targets the specific protein substrates for degradation by the proteasome. Thus, E3 ubiquitin ligase alone or in complex with an E2 ubiquitin conjugating enzyme is responsible for the transfer of ubiquitin to targeted proteins. In general, the ubiquitin ligase is involved in polyubiquitination such that a second ubiquitin is attached to the first; a third is attached to the second, and so forth. Polyubiquitination marks proteins for degradation by the proteasome. However, there are some ubiquitination events that are limited to mono-ubiquitination, in which only a single ubiquitin is added by the ubiquitin ligase to a substrate molecule. Mono-ubiquitinated proteins are not targeted to the proteasome for degradation but may instead be altered in their cellular location or function, for example, via binding other proteins that have domains capable of binding ubiquitin. Further complicating matters, different lysines on ubiquitin can be targeted by an E3 to make chains. The most common lysine is Lys48 on the ubiquitin chain. This is the lysine used to make polyubiquitin, which is recognized by the proteasome.

As used herein, the term “alkyl”, by itself or as part of another substituent, means, unless otherwise stated, a straight or branched chain hydrocarbon radical having up to twelve carbon atoms. In some embodiments, the number of carbon atoms is designated (i.e., C₁-C₈ means one to eight carbons). Examples of alkyl groups include methyl, ethyl, n-propyl, iso-propyl, n-butyl, t-butyl, iso-butyl, sec-butyl, n-pentyl, n-hexyl, n-heptyl, n-octyl, and the like. Alkyl groups may be optionally substituted as provided herein. In some embodiments, the alkyl group is a C₁-C₆ alkyl; in some embodiments, it is a C₁-C₄ alkyl.

When a range of carbon atoms is used herein, for example, C₁-C₆, all ranges, as well as individual numbers of carbon atoms are encompassed. For example, “C₁-C₃” includes C₁-C₃, C₁-C₂, C₂-C₃, C1, C2, and C3.

The term “optionally substituted”, as used in combination with a substituent defined herein, means that the substituent may, but is not required to be, substituted with one or more suitable functional groups or other substituents as provided herein. For example, a substituent may be optionally substituted with one or more of: halo, cyano, C₁₋₆ alkyl, C₃₋₆ cycloalkyl, C₂₋₆ alkenyl, C₂₋₆ alkynyl, halo(C₁₋₆)alkyl, C₁₋₆ alkoxy, halo(C₁₋₆ alkoxy), C₁₋₆ alkylthio, C₁₋₆ alkylamino, NH₂, NH(C₁₋₆ alkyl), N(C₁₋₆ alkyl)₂, NH(C₁₋₆alkoxy), N(C₁₋₆ alkoxy)₂, —C(O)NHC₁₋₆ alkyl, —C(O)N(C₁₋₆ alkyl)₂, —C(O)NH₂, —C(O)C₁₋₆ alkyl, —C(O)₂C₁₋₆ alkyl, —NHCO(C₁₋₆ alkyl), —N(C₁₋₆ alkyl)CO(C₁₋₆ alkyl), —S(O)C₁₋₆ alkyl, —S(O)₂C₁₋₆ alkyl, oxo, 6-12 membered aryl, benzyl, pyridinyl, pyrazolyl, thiazolyl, isothiazolyl, or other 5 to 12 membered heteroaryl groups. In some embodiments, each of the above optional substituents are themselves optionally substituted by one or two groups.

The term “cycloalkyl” as used herein refers to a 3-12 membered cyclic alkyl group, and includes bridged (e.g., adamantine) and spirocycles (e.g., spiro[3.5]nonane). Cycloalkyl groups may be fully saturated or partially unsaturated. The term “cycloalkyl” also includes multiple condensed ring systems (e.g., ring systems comprising 2, 3 or 4 rings) wherein a single cycloalkyl ring (as defined above) can be condensed with one or more groups selected from heterocycles, carbocycles, aryls, or heteroaryls to form the multiple condensed ring system. Such multiple condensed ring systems may be optionally substituted with one or more (e.g., 1, 2, 3 or 4) oxo groups on the carbocycle or heterocycle portions of the multiple condensed ring. The rings of the multiple condensed ring system can be connected to each other via fused, spiro and bridged bonds when allowed by valency requirements. It is to be understood that the individual rings of the multiple condensed ring system may be connected in any order relative to one another. It is also to be understood that the point of attachment of a multiple condensed ring system (as defined above for a cycloalkyl) can be at any position of the cycloalkylic ring. Examples of cycloalkyl groups include cyclopropyl, cyclobutyl, cyclopentyl, cycloheptyl, cyclohexyl, cycloheptyl, cyclooctyl, indenyl, bicyclo[2.2.1]heptanyl, bicyclo[3.1.1]heptanyl, bicyclo[4.1.0]heptanyl, spiro[3.3]heptanyl, and spiro[3.4]octanyl. In some embodiments, the cycloalkyl group is a 3-7 membered cycloalkyl.

The term “akenyl” as used herein refers to C₂-C₁₂ alkyl group that contains at least one carbon-carbon double bond. In some embodiments, the alkenyl group is optionally substituted. In some embodiments, the alkenyl group is a C₂-C₆ alkenyl.

The term “akynyl” as used herein refers to C₂-C₁₂ alkyl group that contains at least one carbon-carbon triple bond. In some embodiments, the alkenyl group is optionally substituted. In some embodiments, the alkynyl group is a C₂-C₆ alkynyl.

The terms “alkoxy,” “alkylamino” and “alkylthio”, are used in their conventional sense, and refer to those alkyl groups attached to the remainder of the molecule via an oxygen atom (“oxy”), an amino group (“amino”) or thio group. The term “alkylamino” includes mono- di-alkylamino groups, the alkyl portions can be the same or different.

The terms “halo” or “halogen”, by itself or as part of another substituent, means a fluorine, chlorine, bromine, or iodine atom.

The term “heteroalkyl” refers to an alkyl group in which one or more carbon atom has been replaced by a heteroatom selected from S, O, P and N. Exemplary heteroalkyls include alkyl ethers, secondary and tertiary alkyl amines, alkyl amides, alkyl sulfides, and the like. The group may be a terminal group or a bridging group. As used herein reference to the normal chain when used in the context of a bridging group refers to the direct chain of atoms linking the two terminal positions of the bridging group.

The term “aryl” as used herein refers to a single, all carbon aromatic ring or a multiple condensed all carbon ring system wherein at least one of the rings is aromatic. For example, in certain embodiments, an aryl group has 6 to 12 carbon atoms. Aryl includes a phenyl radical. Aryl also includes multiple condensed ring systems (e.g., ring systems comprising 2, 3 or 4 rings) having about 9 to 12 carbon atoms in which at least one ring is aromatic and wherein the other rings may be aromatic or not aromatic. Such multiple condensed ring systems are optionally substituted with one or more (e.g., 1, 2 or 3) oxo groups on any carbocycle portion of the multiple condensed ring system. The rings of the multiple condensed ring system can be connected to each other via fused, spiro and bridged bonds when allowed by valency requirements. It is to be understood that the point of attachment of a multiple condensed ring system, as defined above, can be at any position of the aromatic ring. Non-limiting examples of aryl groups include, but are not limited to, phenyl, indenyl, naphthyl, 1, 2,3,4-tetrahydronaphthyl, and the like.

The term “heteroaryl” as used herein refers to a single aromatic ring that has at least one atom other than carbon in the ring, wherein the atoms are selected from the group consisting of oxygen, nitrogen and sulfur; “heteroaryl” also includes multiple condensed ring systems that have at least one such aromatic ring, which multiple condensed ring systems are further described below. Thus, “heteroaryl” includes single aromatic rings of from about 1 to 6 carbon atoms and about 1-4 heteroatoms selected from the group consisting of oxygen, nitrogen and sulfur. The sulfur and nitrogen atoms may also be present in an oxidized form provided the ring is aromatic. Exemplary heteroaryl ring systems include but are not limited to pyridyl, pyrimidinyl, oxazolyl or furyl. “Heteroaryl” also includes multiple condensed ring systems (e.g., ring systems comprising 2, 3 or 4 rings) wherein a heteroaryl group, as defined above, is condensed with one or more rings selected from heteroaryls (to form for example a naphthyridinyl such as 1,8-naphthyridinyl), heterocycles, (to form for example a 1, 2, 3, 4-tetrahydronaphthyridinyl such as 1,2,3,4-tetrahydro-1,8-naphthyridinyl), carbocycles (to form for example 5,6,7,8-tetrahydroquinolyl) and aryls (to form for example indazolyl) to form the multiple condensed ring system. Thus, a heteroaryl (a single aromatic ring or multiple condensed ring system) has about 1-20 carbon atoms and about 1-6 heteroatoms within the heteroaryl ring. A heteroaryl (a single aromatic ring or multiple condensed ring system) can also have about 5 to 12 or about 5 to 10 members within the heteroaryl ring. Multiple condensed ring systems may be optionally substituted with one or more (e.g., 1, 2, 3 or 4) oxo groups on the carbocycle or heterocycle portions of the condensed ring. The rings of a multiple condensed ring system can be connected to each other via fused, spiro and bridged bonds when allowed by valency requirements. It is to be understood that the individual rings of the multiple condensed ring system may be connected in any order relative to one another. It is also to be understood that the point of attachment of a multiple condensed ring system (as defined above for a heteroaryl) can be at any position of the heteroaryl ring. It is also to be understood that the point of attachment for a heteroaryl or heteroaryl multiple condensed ring system can be at any suitable atom of the heteroaryl ring including a carbon atom and a heteroatom (e.g., a nitrogen). Exemplary heteroaryls include but are not limited to pyridyl, pyrrolyl, pyrazinyl, pyrimidinyl, pyridazinyl, pyrazolyl, thienyl, indolyl, imidazolyl, oxazolyl, isoxazolyl, thiazolyl, furyl, oxadiazolyl, thiadiazolyl, quinolyl, isoquinolyl, benzothiazolyl, benzoxazolyl, indazolyl, quinoxalyl, quinazolyl, 5,6,7,8-tetrahydroisoquinolinyl benzofuranyl, benzimidazolyl, thianaphthenyl, pyrrolo[2,3-b]pyridinyl, quinazolinyl-4(3H)-one, triazolyl, 4,5,6,7-tetrahydro-1H-indazole and 3b,4,4a,5-tetrahydro-1H-cyclopropa[3,4]cyclo-penta[1,2-c]pyrazole. In one embodiment the term “heteroaryl” refers to a single aromatic ring containing at least one heteroatom. For example, the term includes 5-membered and 6-membered monocyclic aromatic rings that include one or more heteroatoms. Non-limiting examples of heteroaryl include but are not limited to pyridyl, furyl, thiazole, pyrimidine, oxazole, and thiadiazole.

The term “heterocyclyl” or “heterocycle” as used herein refers to a single saturated or partially unsaturated ring that has at least one atom other than carbon in the ring, wherein the atom is selected from the group consisting of oxygen, nitrogen and sulfur; the term also includes multiple condensed ring systems that have at least one such saturated or partially unsaturated ring, which multiple condensed ring systems are further described below. Thus, the term includes single saturated or partially unsaturated rings (e.g., 3, 4, 5, 6 or 7-membered rings) from about 1 to 6 carbon atoms and from about 1 to 3 heteroatoms selected from the group consisting of oxygen, nitrogen and sulfur in the ring. The ring may be substituted with one or more (e.g., 1, 2 or 3) oxo groups and the sulfur and nitrogen atoms may also be present in their oxidized forms. Exemplary heterocycles include but are not limited to azetidinyl, tetrahydrofuranyl and piperidinyl. The term “heterocycle” also includes multiple condensed ring systems (e.g., ring systems comprising 2, 3 or 4 rings) wherein a single heterocycle ring (as defined above) can be condensed with one or more groups selected from heterocycles (to form for example a 1,8-decahydronapthyridinyl), carbocycles (to form for example a decahydroquinolyl) and aryls to form the multiple condensed ring system. Thus, a heterocycle (a single saturated or single partially unsaturated ring or multiple condensed ring system) has about 2-20 carbon atoms and 1-6 heteroatoms within the heterocycle ring. Such multiple condensed ring systems may be optionally substituted with one or more (e.g., 1, 2, 3 or 4) oxo groups on the carbocycle or heterocycle portions of the multiple condensed ring. The rings of the multiple condensed ring system can be connected to each other via fused, spiro and bridged bonds when allowed by valency requirements. It is to be understood that the individual rings of the multiple condensed ring system may be connected in any order relative to one another. Accordingly, a heterocycle (a single saturated or single partially unsaturated ring or multiple condensed ring system) has about 3-20 atoms including about 1-6 heteroatoms within the heterocycle ring system. It is also to be understood that the point of attachment of a multiple condensed ring system (as defined above for a heterocylyl) can be at any position of the heterocyclic ring. It is also to be understood that the point of attachment for a heterocycle or heterocycle multiple condensed ring system can be at any suitable atom of the heterocyclic ring including a carbon atom and a heteroatom (e.g., a nitrogen). In one embodiment the term heterocycle includes a C₂₋₂₀ heterocycle. In one embodiment the term heterocycle includes a C₂₋₇ heterocycle. In one embodiment the term heterocycle includes a C₂₋₅ heterocycle. In one embodiment the term heterocycle includes a C₂₋₄ heterocycle. Exemplary heterocycles include, but are not limited to aziridinyl, azetidinyl, pyrrolidinyl, piperidinyl, homopiperidinyl, morpholinyl, thiomorpholinyl, piperazinyl, tetrahydrofuranyl, dihydrooxazolyl, tetrahydropyranyl, tetrahydrothiopyranyl, 1,2,3,4-tetrahydroquinolyl, benzoxazinyl, dihydrooxazolyl, chromanyl, 1,2-dihydropyridinyl, 2,3-dihydrobenzofuranyl, 1,3-benzodioxolyl, 1,4-benzodioxanyl, spiro[cyclopropane-1,1′-isoindolinyl]-3′-one, isoindolinyl-1-one, 2-oxa-6-azaspiro[3.3]heptanyl, imidazolidin-2-one N-methylpiperidine, imidazolidine, pyrazolidine, butyrolactam, valerolactam, imidazolidinone, hydantoin, dioxolane, phthalimide, 1,4-dioxane, thiomorpholine, thiomorpholine-S-oxide, thiomorpholine-S,S-oxide, pyran, 3-pyrroline, thiopyran, pyrone, tetrhydrothiophene, quinuclidine, tropane, 2-azaspiro[3.3]heptane, 2-azaspiro[3.5]nonane, 7-azaspiro[3.5]nonane, 2,7-diazaspiro[3.5]nonane, (1R,5S)-3-azabicyclo[3.2.1]octane, (1s,4s)-2-azabicyclo[2.2.2]octane, (1R,4R)-2-oxa-5-azabicyclo[2.2.2]octane and pyrrolidin-2-one. In one embodiment the term “heterocycle” refers to a monocyclic, saturated or partially unsaturated, 3-8 membered ring having at least one heteroatom. For example, the term includes a monocyclic, saturated or partially unsaturated, 4, 5, 6, or 7 membered ring having at least one heteroatom. Non-limiting examples of heterocycle include aziridine, azetidine, pyrrolidine, piperidine, piperidine, piperazine, oxirane, morpholine, and thiomorpholine. The term “9- or 10-membered heterobicycle” as used herein refers to a partially unsaturated or aromatic fused bicyclic ring system having at least one heteroatom. For example, the term 9- or 10-membered heterobicycle includes a bicyclic ring system having a benzo ring fused to a 5-membered or 6-membered saturated, partially unsaturated, or aromatic ring that contains one or more heteroatoms.

As used herein, the term “heteroatom” is meant to include oxygen (O), nitrogen (N), sulfur (S) and silicon (Si). The nitrogen and sulfur can be in an oxidized form when feasible.

As used herein, the term “chiral” refers to molecules which have the property of non-superimposability of the mirror image partner, while the term “achiral” refers to molecules which are superimposable on their mirror image partner.

As used herein, the term “stereoisomers” refers to compounds which have identical chemical constitution but differ with regard to the arrangement of the atoms or groups in space, e.g., enantiomers, diastereomers, tautomers.

The term “patient” or “subject” is used throughout the specification to describe an animal, preferably a human or a domesticated animal, to whom treatment, including prophylactic treatment, with the compositions according to the present disclosure is provided. For treatment of those infections, conditions or disease states which are specific for a specific animal such as a human patient, the term patient refers to that specific animal, including a domesticated animal such as a dog or cat or a farm animal such as a horse, cow, sheep, etc. In general, in the present disclosure, the term patient refers to a human patient unless otherwise stated or implied from the context of the use of the term.

The term “effective” is used to describe an amount of a compound, composition or component which, when used within the context of its intended use, effects an intended result. The term effective subsumes all other effective amount or effective concentration terms, which are otherwise described or used in the present application.

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, e.g., in humans.

“Pharmaceutically acceptable salt” refers to a salt of a compound of the disclosure that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate and the like.

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of an agent and that is compatible therewith. Examples of excipients include calcium carbonate, calcium phosphate, various sugars and types of starch, cellulose derivatives, gelatin, vegetable oils, and polyethylene glycols.

A “solvate” refers to a physical association of a compound of Formula I with one or more solvent molecules.

“Treating” or “treatment” of any disease or disorder refers, in one embodiment, to ameliorating the disease or disorder (e.g., arresting or reducing the development of the disease or at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to delaying the onset of the disease or disorder.

In one embodiment, the disclosure is directed to a compound of Formula (I):

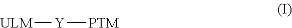

or a pharmaceutically acceptable salt or solvate thereof,
wherein Y-PTM is a moiety of Formula IA:

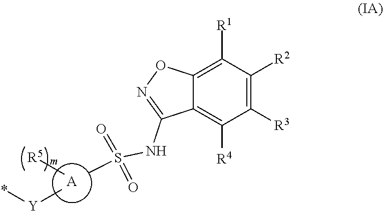

wherein
   Y is a covalent bond, or chemical moiety that links PTM and ULM;
   * is a point of attachment to ULM;
   Ring A is a C₆-C₁₀ membered aryl group or a 5-10 membered heteroaryl group;
   R¹ is H or 5-6 membered heteroaryl optionally substituted by methyl;
   R² is H, C₁₋₄alk-O—C₁₋₄alkyl, or —(C(R⁸)₂)ₙ-(5-9 membered heteroaryl) optionally substituted by halogen, C₁-C₃ alkyl, —CH₂OH, or —OH;
   R³ is H, halogen, C₁-C₄ alkyl, cyclopropyl, haloalkyl, C₁-C₄ alkoxy, or haloalkoxy;
   R⁴ is H, halogen, C₁-C₄ alkyl, cyclopropyl, C₁-C₄ alkoxy, or —O-cyclopropyl;
   each R⁵ is independently H, halogen, oxo, —OH, —CN, —NO₂, —C₁-C₆alkyl, —C₂-C₆alkenyl, —C₂-C₆alkynyl, C₀-C₁alk-aryl, C₀-C₁alk-heteroaryl, cycloalkyl, cycloalkenyl, heterocycloalkyl, or heterocycloalkenyl, —OR^{a}, —SR^{a}, —NR^{c}R^{d}, —NR^{a}R^{c}, —C(O)R^{b}, —OC(O)R^{a}, —C(O)OR^{a}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{d})(OR^{c}), or —S(O)₂R^{b};
   each R^{a} is independently H, —C(O)R^{b}, —C(O)OR^{c}, —C(O)NR^{c}R^{d}, —C(═NR^{b})NR^{b}R^{c}, —C(═NOR^{b})R^{b}R^{c}, —C(═NCN)NR^{b}R^{c}, —P(OR^{c})₂, —P(O)R^{c}R^{b}, —P(O)OR^{c}OR^{b}, —S(O)R^{b}, —S(O)NR^{c}R^{d}, —S(O)₂R^{b}, —S(O)₂NR^{c}R^{d}, SiR^{b}₃, —C₁-C₁₀alkyl, —C₁-C₁₀ haloalkyl, —C₂-C₁₀ alkenyl, —C₂-C₁₀ alkynyl, aryl, cycloalkyl, cycloalkenyl, heteroaryl, heterocycloalkyl, or heterocycloalkenyl;
   each R^{b} is independently H, —C₁-C₆ alkyl, —C₁-C₆ haloalkyl, —C₂-C₆ alkenyl, —C₂-C₆ alkynyl, aryl, cycloalkyl, cycloalkenyl, heteroaryl, heterocycloalkyl, or heterocycloalkenyl;
   each R^{c} or R^{d} is independently H, —C₁-C₁₀ alkyl, —C₂-C₆ alkenyl, —C₂-C₆ alkynyl, —OC₁-C₆alkyl, —O-cycloalkyl, aryl, heteroaryl, cycloalkyl, cycloalkenyl, heterocycloalkyl, or heterocycloalkenyl; or
   R^{c} and R^{d}, together with the atom to which they are both attached, form a monocyclic or multicyclic heterocycloalkyl, or a monocyclic or multicyclic heterocyclo-alkenyl group;
   each R⁸ is independently H, halogen, or C₁-C₄ alkyl;
   m is 0, 1, 2, 3, or 4;
   n is 0 or 1; and
   ULM is

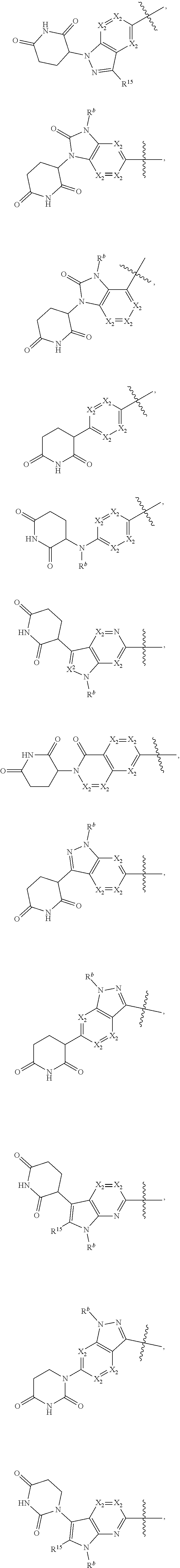

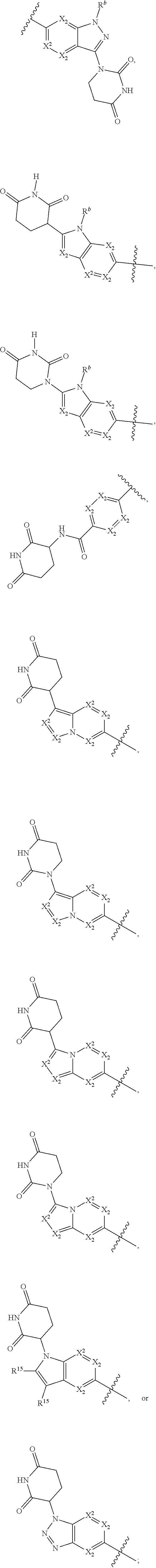

wherein
[symbol] is a point of attachment to Y;
each X₂ is independently N or CR¹⁵; and
each R¹⁵ is independently H, D, halogen, oxo, —OH, —CN, —NO₂, —C₁-C₆alkyl, —C₁-C₆ haloalkyl, —C₂-C₆alkenyl, —C₂-C₆alkynyl, C₀-C₁alk-aryl, C₀-C₁alk-heteroaryl, cycloalkyl, cycloalkenyl, heterocycloalkyl, or heterocycloalkenyl, —OR^{a}, —SR^{a}, —NR^{c}R^{d}, —NR^{a}R^{c}, —C(O)R^{b}, —OC(O)R^{a}, —C(O)OR^{a}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{d})(OR^{c}) or —S(O)₂R^{b};
wherein any of the —C₁-C₆alkyl, C₁-C₈ alkoxide, —C₂-C₆alkenyl, —C₂-C₆alkynyl, aryl, heteroaryl, cycloalkyl, cycloalkenyl, heterocycloalkyl, heterocycloalkenyl, spirocycloalkyl, spiroheterocycloalkyl and spiroheterocycloalkenyl groups in any of R₁—R₅, R₈, R¹⁵, and ring A is optionally substituted by 1-6 R^{f} groups; and
each R^{f} is independently D, oxo, halogen, C₁-C₈ alkoxy, C₁-C₈ alkyl, C₁₋₆ haloalkyl, C₁₋₆ haloalkoxy, —OH, —CN, —NO₂, —C₂₋₆ alkenyl, —C₂₋₆ alkynyl, C₆₋₁₀ aryl, C₅₋₁₂ heteroaryl, C₃₋₈ cycloalkyl, C₃₋₈ cycloalkenyl, C₃₋₈ heterocycloalkyl, C₃₋₈ heterocycloalkenyl, —OR^{a}, —SR^{a}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)R^{c}R^{d}, —P(O)(OR^{b})(OR^{b}), —B(OR^{c})(OR^{d}), —S(O)₂R^{b}, —C(O)NR^{b}OR^{b}, —S(O)₂OR^{b}, —OS(O)₂OR^{b}, or —OPO(OR^{b})(OR^{b}); wherein said C₁-C₈ alkyl is optionally substituted by 1-6 groups selected from D, halogen, —OH, —CN, —OR^{a}, —SR^{a}, —NR^{a}R^{d}, or NR^{c}R^{d}.

In some embodiments, the compounds of Formula I include a protein targeting moiety (PTM). In some embodiments, the Y-PTM in the compounds of Formula I is a moiety of Formula IA

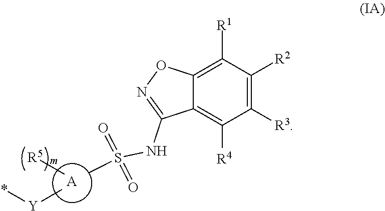

According to the disclosure, Y in Formula IA is a covalent bond, or chemical moiety that links PTM and ULM. In some embodiments, Y in Formula IA is a chemical moiety that links PTM and ULM. In other embodiments, Y in Formula IA is a covalent bond.

According to the disclosure, Ring A in Formula IA is a C₆-C₁₀ membered aryl group or a 5-10 membered heteroaryl group. In some embodiments, Ring A in Formula IA is a C₆-C₁₀ membered aryl group. In some embodiments, Ring A in Formula IA is a phenyl group. In other embodiments, Ring A in Formula IA is a 5-10 membered heteroaryl group.

In some embodiments, Ring A in Formula IA is selected from

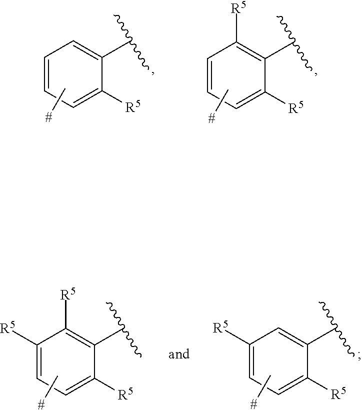

wherein [symbol] is a point of attachment to the sulfonamide group and # is a point of attachment to Y.

In some embodiments, Ring A in Formula IA is

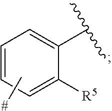

wherein [symbol] is a point of attachment to the sulfonamide group and # is a point of attachment to Y.

In some embodiments, Ring A in Formula IA is

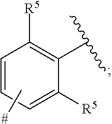

wherein [symbol] is a point of attachment to the sulfonamide group and # is a point of attachment to Y.

In other embodiments, Ring A in Formula IA is

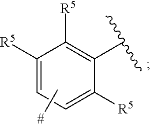

wherein [symbol] is a point of attachment to the sulfonamide group and # is a point of attachment to Y.

In yet other embodiments, Ring A in Formula IA is

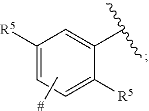

wherein [symbol] is a point of attachment to the sulfonamide group and # is a point of attachment to Y.

In some embodiments, Ring A in Formula IA is selected from

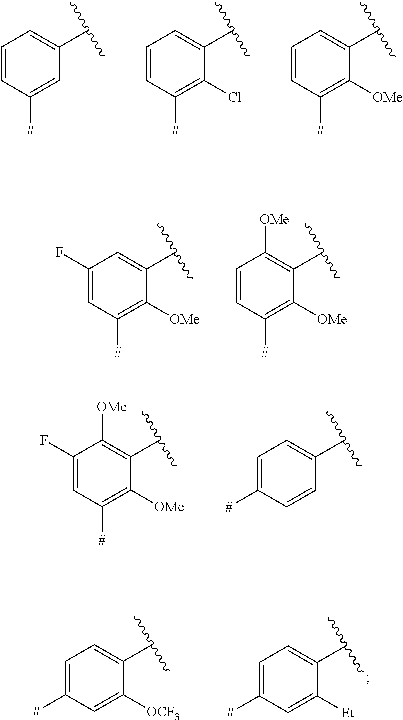

wherein [symbol] is a point of attachment to the sulfonamide group and # is a point of attachment to Y.

In some embodiments, Ring A in Formula IA is

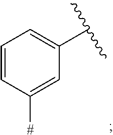

wherein [symbol] is a point of attachment to the sulfonamide group and # is a point of attachment to Y.

In some embodiments, Ring A in Formula IA is

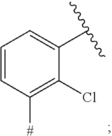

wherein [symbol] is a point of attachment to the sulfonamide group and # is a point of attachment to Y.

In some embodiments, Ring A in Formula IA is

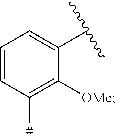

wherein [symbol] is a point of attachment to the sulfonamide group and # is a point of attachment to Y.

In other embodiments, Ring A in Formula IA is

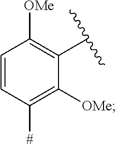

wherein [symbol] is a point of attachment to the sulfonamide group and # is a point of attachment to Y.

In other embodiments, Ring A in Formula IA is

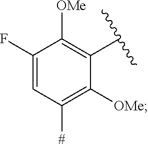

wherein [symbol] is a point of attachment to the sulfonamide group and # is a point of attachment to Y.

In yet other embodiments, Ring A in Formula IA is

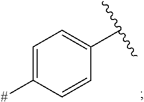

wherein [symbol] is a point of attachment to the sulfonamide group and # is a point of attachment to Y.

In yet other embodiments, Ring A in Formula IA is

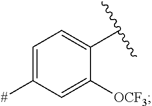

wherein [symbol] is a point of attachment to the sulfonamide group and # is a point of attachment to Y.

In yet other embodiments, Ring A in Formula IA is

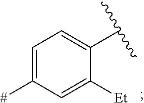

wherein [symbol] is a point of attachment to the sulfonamide group and # is a point of attachment to Y.

According to the disclosure, R¹ in Formula IA is H, D, or 5-6 membered heteroaryl optionally substituted by methyl. In some embodiments, R¹ in Formula IA is H. In some embodiments, R¹ in Formula IA is D. In other embodiments, R¹ in Formula IA is 5-6 membered heteroaryl optionally substituted by methyl.

According to the disclosure, R² in Formula IA is H, D, C₁₋₄alk-O—C₁₋₄alkyl or —(C(R⁸)₂)ₙ-(5-9 membered heteroaryl) optionally substituted by halogen, C₁-C₃ alkyl, —CH₂OH, or —OH. In some embodiments, R² in Formula IA is H. In some embodiments, R² in Formula IA is D. In other embodiments, R² in Formula IA is C₁₋₄alk-O—C₁₋₄alkyl. In other embodiments, R² in Formula IA is CH₂—O—CH₃.

In yet other embodiments, R² in Formula IA is —(C(R⁸)₂)ₙ-(5-9 membered heteroaryl) optionally substituted by halogen, C₁-C₃ alkyl, —CH₂OH, or —OH. In yet other embodiments, R² in Formula IA is —(C(R⁸)₂)ₙ-(5-6 membered heteroaryl) optionally substituted by halogen, C₁-C₃ alkyl, —CH₂OH, or —OH. In some embodiments, when R² in Formula IA is —(C(R⁸)₂)ₙ-(5-6 membered heteroaryl), the 5-6 membered heteroaryl is a pyrazole, a pyrrole, a pyridine or a pyridazine. In some embodiments, when R² in Formula IA is —(C(R⁸)₂)ₙ-(5-6 membered heteroaryl), the 5-6 membered heteroaryl is a pyrazole.

According to the disclosure, R⁸ in Formula IA is H, D, halogen, or C₁-C₄ alkyl. In some embodiments, R⁸ in Formula IA is H. In some embodiments, R⁸ in Formula IA is D. In other embodiments, R⁸ in Formula IA is halogen. In other embodiments, R⁸ in Formula IA is C₁-C₄ alkyl.

According to the disclosure, n in Formula IA is 0 or 1. In some embodiments, n in Formula IA=0. In other embodiments, n in Formula IA=1.

According to the disclosure, R³ in Formula IA is H, D, halogen, C₁-C₄ alkyl, cyclopropyl, haloalkyl, C₁-C₄ alkoxy, or haloalkoxy. In some embodiments, R³ in Formula IA is H. In some embodiments, R³ in Formula IA is D. In some embodiments, R³ in Formula IA is halogen. In some embodiments, R³ in Formula IA is C₁-C₄ alkyl. In other embodiments, R³ in Formula IA is cyclopropyl. In other embodiments, R³ in Formula IA is C₁-C₄ haloalkyl. In other embodiments, R³ in Formula IA is C₁-C₄ alkoxy. In other embodiments, R³ in Formula IA is haloalkoxy.

According to the disclosure, R⁴ in Formula IA is H, D, halogen, C₁-C₄ alkyl, cyclopropyl, C₁-C₄ alkoxy, or —O-cyclopropyl. In some embodiments, R⁴ in Formula IA is H. In some embodiments, R⁴ in Formula IA is D. In some embodiments, R⁴ in Formula IA is halogen. In some embodiments, R⁴ in Formula IA is C₁-C₄ alkyl. In other embodiments, R⁴ in Formula IA is cyclopropyl. In other embodiments, R⁴ in Formula IA is C₁-C₄ alkoxy. In other embodiments, R⁴ is —O-cyclopropyl. In other embodiments, R⁴ in Formula IA is methoxy.

According to the disclosure, each R⁵ in Formula IA is independently H, halogen, oxo, —OH, —CN, —NO₂, —C₁-C₆alkyl, —C₂-C₆alkenyl, —C₂-C₆alkynyl, C₀-C₁alk-aryl, C₀-C₁alk-heteroaryl, cycloalkyl, cycloalkenyl, heterocycloalkyl, or heterocycloalkenyl, —OR^{a}, —SR^{a}, —NR^{c}R^{d}, —NR^{a}R^{c}, —C(O)R^{b}, —OC(O)R^{a}, —C(O)OR^{a}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{d})(OR^{c}) or —S(O)₂R^{b}.

In some embodiments, at least one R⁵ in Formula IA is H. In some embodiments, each R⁵ in Formula IA is D. In some embodiments, at least one R⁵ in Formula IA is halogen. In some embodiments, each R⁵ in Formula IA is C₁-C₄ alkoxy. In some embodiments, at least one R⁵ in Formula IA is C₁-C₄ alkoxy.

In other embodiments, at least one R⁵ in Formula IA is F. In other embodiments, at least one R⁵ in Formula IA is —CN. In other embodiments, at least one R⁵ in Formula IA is C₁-C₄ alkyl. In other embodiments, at least one R⁵ in Formula IA is haloalkyl. In other embodiments, at least one R⁵ in Formula IA is cyclopropyl. In yet other embodiments, at least one R⁵ in Formula IA is haloalkoxy. In yet other embodiments, at least one R⁵ in Formula IA is —O-cyclopropyl. In yet other embodiments, at least one R⁵ in Formula IA is —CH₂—O—CH₃. In yet other embodiments, at least one R⁵ in Formula IA is —C(O)OCH₃. In yet other embodiments, at least one R⁵ in Formula IA is —C(O)N(H)CH₃.

In some embodiments, at least one R⁵ in Formula IA is halogen and at least one R₅ is C₁-C₄ alkoxy. In some embodiments, one R⁵ in Formula IA is halogen and two R₅ are C₁-C₄ alkoxy. In some embodiments, one R⁵ in Formula IA is F and two R₅ are OCH₃.

According to the disclosure, m in Formula IA is 0, 1, 2, 3, or 4. In some embodiments, m is 0. In some embodiments, m in Formula IA is 1. In other embodiments, m in Formula IA is 2. In other embodiments, m in Formula IA is 3. In other embodiments, m in Formula IA is 4.

According to the disclosure, ULM is a small molecule E3 Ubiquitin Ligase binding moiety that binds a Cereblon E3 Ubiquitin Ligase. In some embodiments, ULM is a moiety as described herein.

In some embodiments, ULM is

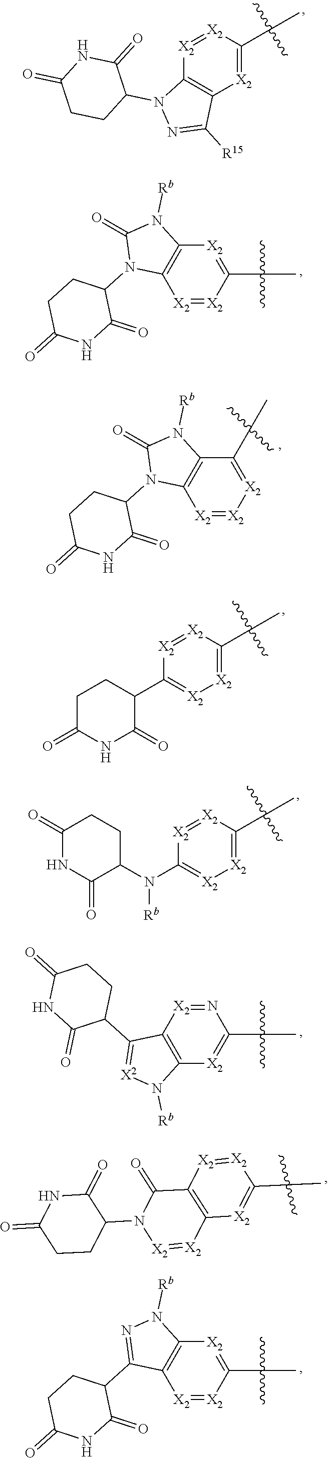

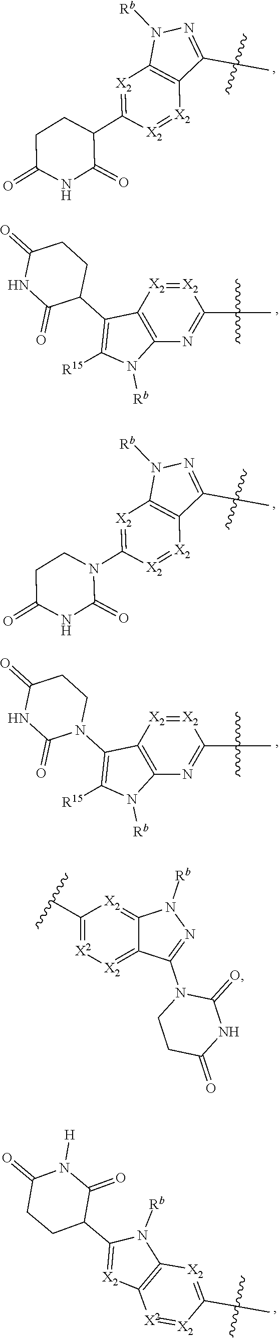

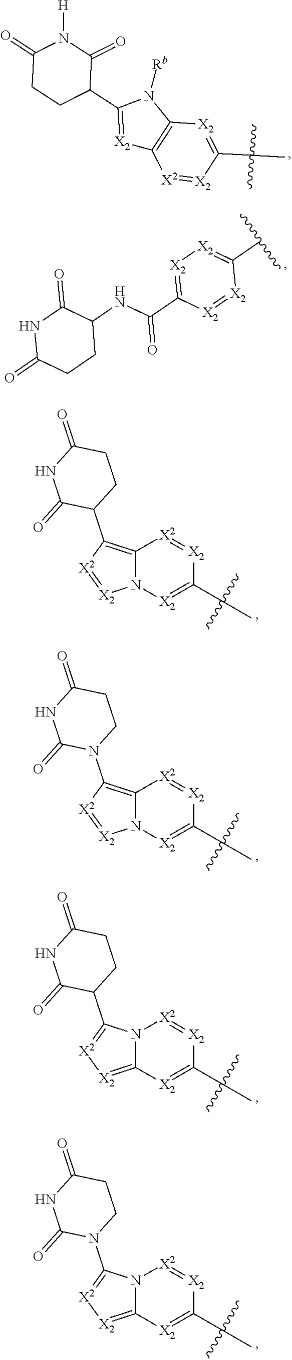

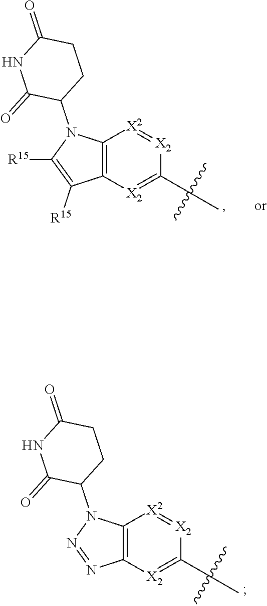

wherein
[symbol] is a point of attachment to Y;
each X₂ is independently N or CR¹⁵; and
each R¹⁵ is independently H, D, halogen, oxo, —OH, —CN, —NO₂, —C₁-C₆alkyl, —C₁-C₆ haloalkyl, —C₂-C₆alkenyl, —C₂-C₆alkynyl, C₀-C₁alk-aryl, C₀-C₁alk-heteroaryl, cycloalkyl, cycloalkenyl, heterocycloalkyl, or heterocycloalkenyl, —OR^{a}, —SR^{a}, —NR^{c}R^{d}, —NR^{a}R^{c}, —C(O)R^{b}, —OC(O)R^{a}, —C(O)OR^{a}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{d})(OR^{c}) or —S(O)₂R^{b}.

In some embodiments, ULM is

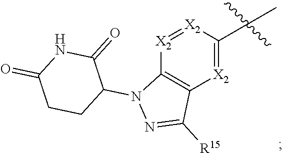

wherein [symbol] is a point of attachment to Y; and each X₂ is independently N or CR¹⁵.

In some embodiments, ULM is

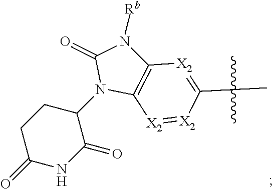

wherein [symbol] is a point of attachment to Y; and each X₂ is independently N or CRIS.

In some embodiments, ULM is

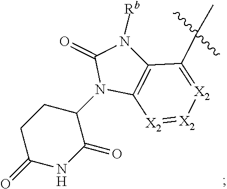

wherein [symbol] is a point of attachment to Y; and each X₂ is independently N or CRIS.

In some embodiments, ULM is

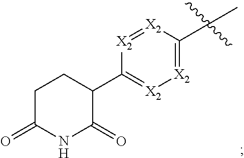

wherein [symbol] is a point of attachment to Y; and each X₂ is independently N or CR¹⁵.

In some embodiments, ULM is

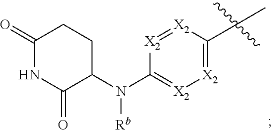

wherein [symbol] is a point of attachment to Y; and each X₂ is independently N or CR¹⁵.

In some embodiments, ULM is

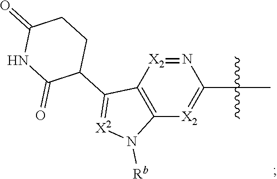

wherein [symbol] is a point of attachment to Y; and each X₂ is independently N or CR¹⁵.

In some embodiments, ULM is

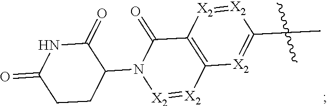

wherein [symbol] is a point of attachment to Y; and each X₂ is independently N or CR¹⁵.

In some embodiments, ULM is

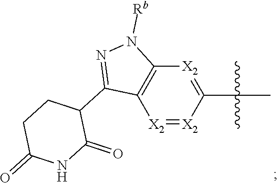

wherein [symbol] is a point of attachment to Y; and each X₂ is independently N or CR¹⁵.

In other embodiments, ULM is

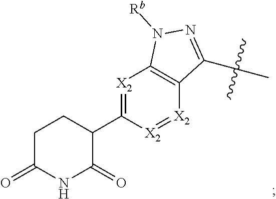

wherein [symbol] is a point of attachment to Y; and each X₂ is independently N or CR¹⁵.

In other embodiments, ULM is

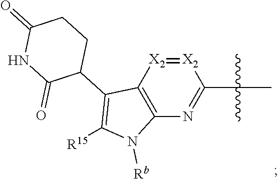

wherein [symbol] is a point of attachment to Y; and each X₂ is independently N or CR¹⁵.

In other embodiments, ULM is

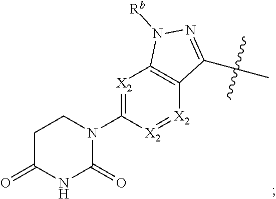

wherein [symbol] is a point of attachment to Y; and each X₂ is independently N or CR¹⁵.

In other embodiments, ULM is

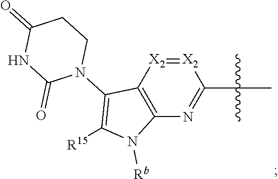

wherein [symbol] is a point of attachment to Y; and each X₂ is independently N or CR¹⁵.

In other embodiments, ULM is

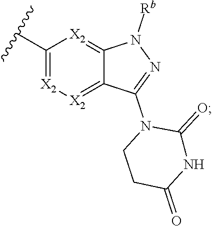

wherein [symbol] is a point of attachment to Y; and each X₂ is independently N or CR¹⁵.

In other embodiments, ULM is

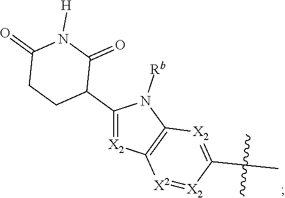

wherein [symbol] is a point of attachment to Y; and each X₂ is independently N or CR¹⁵.

In other embodiments, ULM is

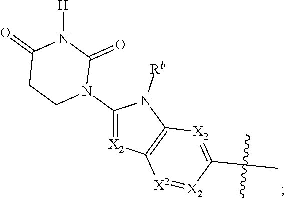

wherein [symbol] is a point of attachment to Y; and each X₂ is independently N or CR¹⁵.

In other embodiments, ULM is

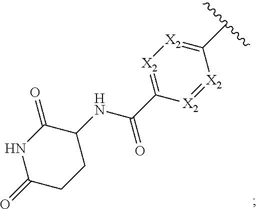

wherein [symbol] is a point of attachment to Y; and each X₂ is independently N or CR¹⁵.

In yet other embodiments, ULM is

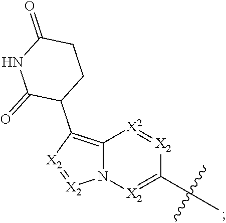

wherein [symbol] is a point of attachment to Y; and each X₂ is independently N or CR¹⁵.

In yet other embodiments, ULM is

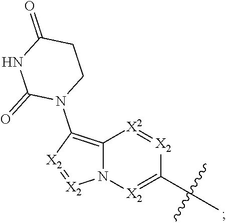

wherein [symbol] is a point of attachment to Y; and each X₂ is independently N or CR¹⁵.

In yet other embodiments, ULM is

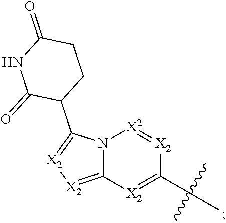

wherein [symbol] is a point of attachment to Y; and each X₂ is independently N or CR¹⁵.

In yet other embodiments, ULM is

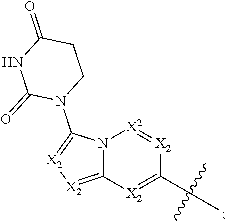

wherein [symbol] is a point of attachment to Y; and each X₂ is independently N or CR¹⁵.

In yet other embodiments, ULM is

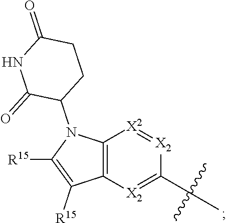

wherein [symbol] is a point of attachment to Y; and each X₂ is independently N or CR¹⁵.

In yet other embodiments, ULM is

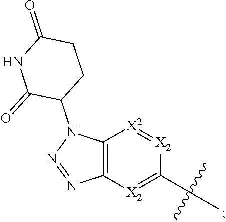

wherein [symbol] is a point of attachment to Y; and each X₂ is independently N or CR¹⁵.

In some embodiments, each X₂ of ULM is independently N or CR¹⁵. In some embodiments, at least one X₂ of ULM is N. In some embodiments, at least one X₂ of ULM is CR^{b}. In some embodiments, each X₂ of ULM is CR¹⁵.

According to the disclosure, each R¹⁵ is independently H, D, halogen, oxo, —OH, —CN, —NO₂, —C₁-C₆alkyl, —C₁-C₆haloalkyl, —C₂-C₆alkenyl, —C₂-C₆alkynyl, C₀-C₁alk-aryl, C₀-C₁alk-heteroaryl, cycloalkyl, cycloalkenyl, heterocycloalkyl, or heterocycloalkenyl, —OR^{a}, —SR^{a}, —NR^{c}R^{d}, —NR^{a}R^{c}, —C(O)R^{b}, —OC(O)R^{a}, —C(O)OR^{a}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{d})(OR^{c}) or —S(O)₂R^{b}.

In some embodiments, each R¹⁵ is H. In some embodiments, at least one R¹⁵ is H. In some embodiments, each R¹⁵ is D. In some embodiments, at least one R¹⁵ is D. In some embodiments, each R¹⁵ is C₁-C₆alkyl. In some embodiments, at least one R¹⁵ is C₁-C₆alkyl. In some embodiments, each R¹⁵ is methyl or ethyl. In some embodiments, at least one R¹⁵ is methyl or ethyl.

In other embodiments, each R¹⁵ is independently selected from halogen, oxo, —OH, —CN, —NO₂, —C₁-C₆haloalkyl, —C₂-C₆alkenyl, —C₂-C₆alkynyl, C₀-C₁alk-aryl, C₀-C₁alk-heteroaryl, cycloalkyl, cycloalkenyl, heterocycloalkyl, or heterocycloalkenyl, —OR^{a}, —SR^{a}, —NR^{c}R^{d}, —NR^{a}R^{c}, —C(O)R^{b}, —OC(O)R^{a}, —C(O)OR^{a}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{d})(OR^{c}) or —S(O)₂R^{b}.

In some embodiments, ULM is

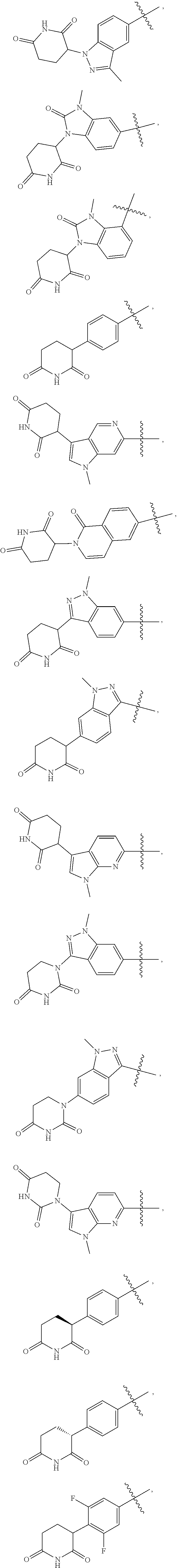

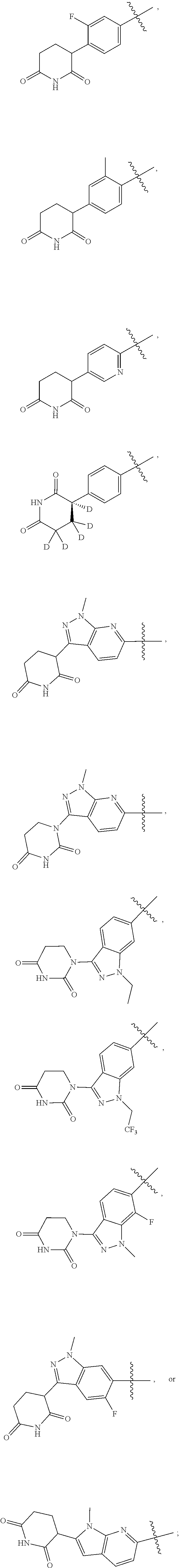

wherein [symbol] is a point of attachment to Y.

In some embodiments, ULM is

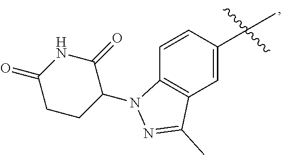

wherein [symbol] is a point of attachment to Y.

In some embodiments, ULM is

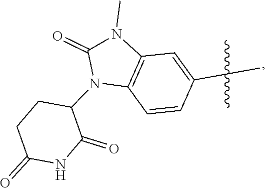

wherein [symbol] is a point of attachment to Y.

In some embodiments, ULM is

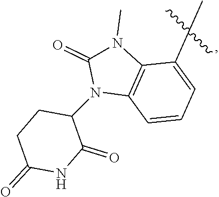

wherein [symbol] is a point of attachment to Y.

In some embodiments, ULM is

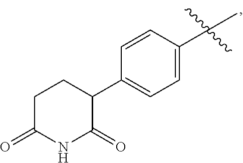

wherein [symbol] is a point of attachment to Y.

In some embodiments, ULM is

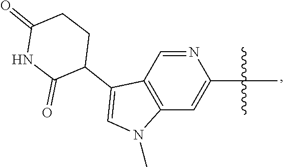

wherein [symbol] is a point of attachment to Y.

In some embodiments, ULM is

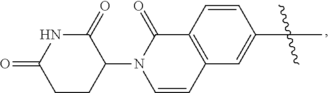

wherein [symbol] is a point of attachment to Y.

In some embodiments, ULM is

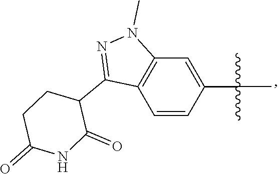

wherein [symbol] is a point of attachment to Y.

In some embodiments, ULM is

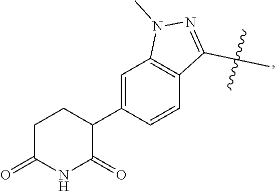

wherein [symbol] is a point of attachment to Y.

In some embodiments, ULM is

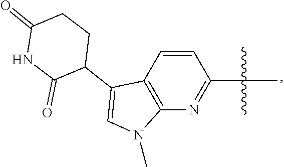

wherein [symbol] is a point of attachment to Y.

In some embodiments, ULM is

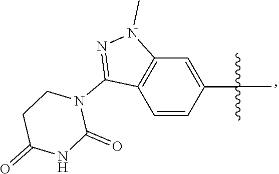

wherein [symbol] is a point of attachment to Y.

In other embodiments, ULM is

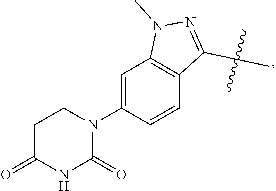

wherein [symbol] is a point of attachment to Y.

In other embodiments, ULM is

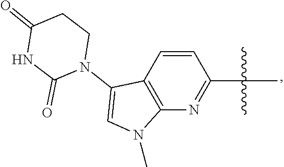

wherein [symbol] is a point of attachment to Y.

In other embodiments, ULM is

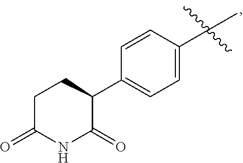

wherein [symbol] is a point of attachment to Y.

In other embodiments, ULM is

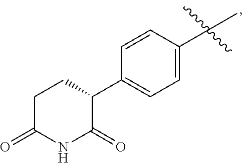

wherein [symbol] is a point of attachment to Y.

In other embodiments, ULM is

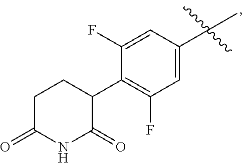

wherein [symbol] is a point of attachment to Y.

In other embodiments, ULM is

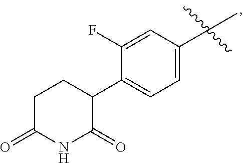

wherein [symbol] is a point of attachment to Y.

In other embodiments, ULM is

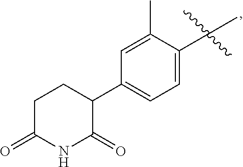

wherein [symbol] is a point of attachment to Y.

In other embodiments, ULM is

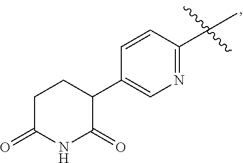

wherein [symbol] is a point of attachment to Y.

In yet other embodiments, ULM is

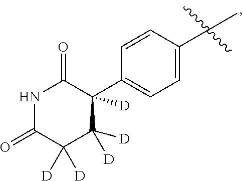

wherein [symbol] is a point of attachment to Y.

In yet other embodiments, ULM is

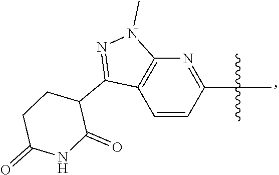

wherein [symbol] is a point of attachment to Y.

In yet other embodiments, ULM is

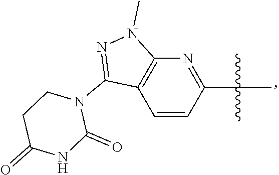

wherein [symbol] is a point of attachment to Y.

In yet other embodiments, ULM is

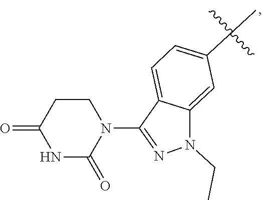

wherein [symbol] is a point of attachment to Y.

In yet other embodiments, ULM is

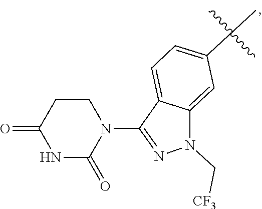

wherein [symbol] is a point of attachment to Y.

In yet other embodiments, ULM is

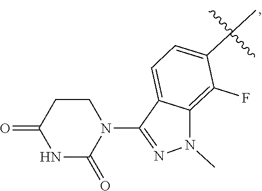

wherein [symbol] is a point of attachment to Y.

In yet other embodiments, ULM is

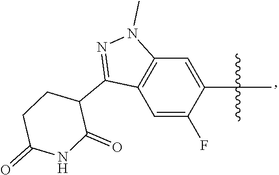

wherein [symbol] is a point of attachment to Y.

In yet other embodiments, ULM is

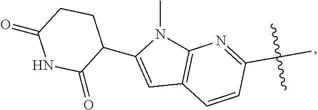

wherein [symbol] is a point of attachment to Y.

Chemical moieties that are used to link PTM and ULM moieties are known in the art. These moieties are sometimes referred to as “linkers” in the art. In some embodiments, Y in Formula IA is a chemical moiety that is used to link a PTM and ULM that is known in the art.

In some embodiments, Y in Formula IA is a chemical moiety that is used to link a PTM and ULM as described in U.S. Patent Application Publication No. 2019/0300521, the entirety of which is incorporated by reference herein.

In other embodiments, Y in Formula IA is a chemical moiety that is used to link a PTM and ULM as described in U.S. Patent Application Publication No. 2019/0255066, the entirety of which is incorporated by reference herein.

In other embodiments, Y in Formula IA is a chemical moiety that is used to link a PTM and ULM as described in WO 2019/084030, the entirety of which is incorporated by reference herein.

In other embodiments, Y in Formula IA is a chemical moiety that is used to link a PTM and ULM as described in WO 2019/084026, the entirety of which is incorporated by reference herein.

In some embodiments, Y in Formula IA is a chemical structural unit represented by the formula:

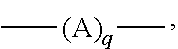

wherein:
q is an integer from 1 to 14;
   each A is independently selected from the group consisting of CR^{1a}R^{1b}, O, S, SO, SO₂, NR^{1c}, SO₂NR^{1c}, SONR^{1c}, SO(═NR^{1c}), SO(═NR^{1c})NR^{1d}, CONR^{1c}, NR^{1c}CONR^{1d}, NR^{1c}C(O)O, NR^{1c}SO₂NR^{1d}, CO, CR^{1a}═CR^{1b}, C≡C, SiR^{1a}R^{1b}, P(O)R^{1a}, P(O)OR^{1a}, (CR^{1a}R^{1b})₁₋₄, —(CR^{1a}R^{1b})₁₋₄O(CR^{1a}R^{1b})₁₋₄, —(CR^{1a}R^{1b})₁₋₄S(CR^{1a}R^{1b})₁₋₄, —(CR^{1a}R^{1b})₁₋₄NR^{1c}(CR^{1a}R^{1b})₁₋₄, NR^{1c}C(═NCN)NR^{1d}NR^{1c}C(═NCN), NR^{1c}C(═CNO₂)NR^{1d}, 3-11 membered cycloalkyl, optionally substituted with 1-6 R^{1a} or R^{1b} groups, 3-11 membered heterocyclyl optionally substituted with 1-6 R^{1a} or R^{1b} groups, aryl optionally substituted with 1-6 R^{1a} or R^{1b} groups, heteroaryl optionally substituted with 1-6 R^{1a} or R^{1b} groups, and R^{1a}, R^{1b}, R^{1c}, R^{1d} and R^{1c} are each independently, —H, D, -halo, —C₁-C₈alkyl, —C₁-C₆haloalkyl, —O—C₁-C₈alkyl, —S—C₁-C₈alkyl, —NHC₁-C₈alkyl, —N(C₁-C₈alkyl)₂, 3-11 membered cycloalkyl, aryl, heteroaryl, 3-11 membered heterocyclyl, —O-(3-11 membered cycloalkyl), —S-(3-11 membered cycloalkyl), NH-(3-11 membered cycloalkyl), N(3-11 membered cycloalkyl)₂, N-(3-11 membered cycloalkyl)(C₁-C₈alkyl), —OH, —NH₂, —SH, —SO₂C₁-C₈alkyl, —SO₂-aryl, —SO₂-heteroaryl, SO(NH)C₁-C₈alkyl, P(O)(OC₁-C₈alkyl)(C₁-C₈alkyl), —P(O)(OC₁-C₈alkyl)₂, —C≡C—C₁-C₈alkyl, —C≡CH, —CH═CH(C₁-C₈alkyl), —C(C₁-C₈alkyl)═CH(C₁-C₈alkyl), —C(C₁-C₈alkyl)═C(C₁-C₈alkyl)₂, —Si(OH)₃, —Si(C₁-C₈alkyl)₃, —Si(OH)(C₁-C₈alkyl)₂, —C(O)C₁-C₈alkyl, —C(O)OC₁-C₈alkyl, —CO₂H, —CN, —CF₃, —CHF₂, —CH₂F, —NO₂, —SF₅, —SO₂NHC₁-C₈alkyl, —SO₂N(C₁-C₈alkyl)₂, —SO(NH)NHC₁-C₈alkyl, —SO(NH)N(C₁-C₈alkyl)₂, —SONHC₁-C₈alkyl, —SON(C₁-C₈alkyl)₂, —CONHC₁-C₈alkyl, —CON(C₁-C₈alkyl)₂, —N(C₁-C₈alkyl)CONH(C₁-C₈alkyl), —N(C₁-C₈alkyl)CON(C₁-C₈alkyl)₂, —NHCONH(C₁-C₈alkyl), —NHCON(C₁-C₈alkyl)₂, —NHCONH₂, —N(C₁-C₈alkyl)SO₂NH(C₁-C₈alkyl), —N(C₁-C₈alkyl)SO₂N(C₁-C₈alkyl)₂, —NHSO₂NH(C₁-C₈alkyl), —NHSO₂N(C₁-C₈alkyl)₂, or —NHSO₂NH₂; or
where the context permits, R^{1a} or R^{1b}, are linked to other groups, or to each other, to form a cycloalkyl and/or a heterocyclyl moiety, optionally substituted with 0-4 R^{1c} groups.

In these embodiments, q represents the number of connected A groups. For example, when q=1, -(A)_{q}- is -A₁-; when q=2, -(A)_{q}- is -A₁-A₂-; when q=3, -(A)_{q}- is -A₁-A₂-A₃-; when q=4, -(A)_{q}- is -A₁-A₂-A₃-A₄-; when q=5, -(A)_{q}- is -A₁-A₂-A₃-A₄-A₅-; when q=6, -(A)_{q}- is -A₁-A₂-A₃-A₄-A₅-A₆-; when q=7, -(A)_{q}- is -A₁-A₂-A₃-A₄-A₅-A₆-A₇-; when q=8, -(A)_{q}- is -A₁-A₂-A₃-A₄-A₅-A₆-A₇-A₈-; when q=9, -(A)_{q}- is -A₁-A₂-A₃-A₄-A₅-A₆-A₇-A₈-A₉-; when q=10, -(A)_{q}- is -A₁-A₂-A₃-A₄-A₅-A₆-A₇-A₈-A₉-A₁₀-; when q=11, -(A)_{q}- is -A₁-A₂-A₃-A₄-A₅-A₆-A₇-A₈-A₉-A₁₀-A₁₁-; when q=12, -(A)_{q}- is -A₁-A₂-A₃-A₄-A₅-A₆-A₇-A₈-A₉-A₁₀-A₁₁-A₁₂-; when q=13, -(A)_{q}- is -A₁-A₂-A₃-A₄-A₅-A₆-A₇-A₈-A₉-A₁₀-A₁₁-A₁₂-A₁₃-; and when q=14, -(A)_{q}- is -A₁-A₂-A₃-A₄-A₅-A₆-A₇-A₈-A₉-A₁₀-A₁₁-A₁₂-A₁₃-A₁₄-.

In some embodiments, q=4 and Y in Formula IA is a chemical moiety represented by the formula: -A₁-A₂-A₃-A₄-, wherein each of A₁₄ is independently selected from the group consisting of O, S, SO, SO₂, NR^{1c}, SO₂NR^{1c}, SONR^{1c}, SO(═NR^{1c}), SO(═NR^{1c})NR^{1d}, CONR^{1c}, NR^{1c}CONR^{1d}, NR^{1c}C(O)O, NR^{1c}SO₂NR^{1d}, CO, CR^{1a}═CR^{1b}, C≡C, SiR^{1a}R^{1b}, P(O)R^{1a}, P(O)OR^{1a}, (CR^{1a}R^{1b})₁₋₄, —(CR^{1a}R^{1b})₁₋₄O(CR^{1a}R^{1b})₁₋₄, —(CR^{1a}R^{1b})₁₋₄S(CR^{1a}R^{1b})₁₋₄, —(CR^{1a}R^{1b})₁₋₄NR^{1c}(CR^{1a}R^{1b})₁₋₄, optionally substituted 3-11 membered cycloalkyl, 3-11 membered heterocyclyl, aryl, and heteroaryl;
wherein R^{1a} and R^{1b} are each independently selected from the group consisting of —H, D, -halo, —C₁-C₈alkyl, —O—C₁-C₈alkyl, —C₁-C₆haloalkyl, —S—C₁-C₈alkyl, —NHC₁-C₈alkyl, —N(C₁-C₈alkyl)₂, 3-11 membered cycloalkyl, aryl, heteroaryl, 3-11 membered heterocyclyl, —O-(3-11 membered cycloalkyl), —S-(3-11 membered cycloalkyl), NH-(3-11 membered cycloalkyl), N(3-11 membered cycloalkyl)₂, N-(3-11 membered cycloalkyl)(C₁-C₈alkyl), —OH, —NH₂, —SH, —SO₂C₁-C₈alkyl, —SO₂-aryl, —SO₂-heteroaryl, SO(NH)C₁-C₈alkyl, P(O)(OC₁-C₈alkyl)(C₁-C₈alkyl), —P(O)(OC₁-C₈alkyl)₂, —C≡C—C₁-C₈alkyl, —C≡CH, —CH═CH(C₁-C₈alkyl), —C(C₁-C₈alkyl)═CH(C₁-C₈alkyl), —C(C₁-C₈alkyl)═C(C₁-C₈alkyl)₂, —Si(OH)₃, —Si(C₁-C₈alkyl)₃, —Si(OH)(C₁-C₈alkyl)₂, —C(O)C₁-C₈alkyl, —C(O)OC₁-C₈alkyl, —CO₂H, —CN, —NO₂, —SF₅, —SO₂NHC₁-C₈alkyl, —SO₂N(C₁-C₈alkyl)₂, —SO(NH)NHC₁-C₈alkyl, —SO(NH)N(C₁-C₈alkyl)₂, —SONHC₁-C₈alkyl, —SON(C₁-C₈alkyl)₂, —CONHC₁-C₈alkyl, —CON(C₁-C₈alkyl)₂, —N(C₁-C₈alkyl)CONH(C₁-C₈alkyl), —N(C₁-C₈alkyl)CON(C₁-C₈alkyl)₂, —NHCONH(C₁-C₈alkyl), —NHCON(C₁-C₈alkyl)₂, —NHCONH₂, —N(C₁-C₈alkyl)SO₂NH(C₁-C₈alkyl), —N(C₁-C₈alkyl)SO₂N(C₁-C₈alkyl)₂, —NHSO₂NH(C₁-C₈alkyl), —NHSO₂N(C₁-C₈alkyl)₂, or —NHSO₂NH₂; and
R^{1c} and R^{1d} are each independently selected from the group consisting of H, D, optionally substituted C₁₋₄ alkyl, C₃₋₈ cyclcoalkyl, C₃₋₈ heterocyclcoalkyl, aryl, or heteroaryl.

In other embodiments, q=3 and Y in Formula IA is a chemical moiety represented by the formula: -A₁-A₂-A₃-, wherein each of A₁₋₃ is independently selected from the group consisting of O, S, SO, SO₂, NR^{1c}, SO₂NR^{1c}, SONR^{1c}, SO(═NR^{1c}), SO(═NR^{1c})NR^{1d}, CONR^{c}, NR^{1c}CONR^{1d}, NR^{1c}C(O)O, NR^{1c}SO₂NR^{1d}, CO, CR^{1a}═CR^{1b}, C≡C, SiR^{1a}R^{1b}, P(O)R^{1a}, P(O)OR^{1a}, (CR^{1a}R^{1b})₁₋₄, —(CR^{1a}R^{1b})₁₋₄O(CR^{1a}R^{1b})₁₋₄, —(CR^{1a}R^{1b})₁₋₄S(CR^{1a}R^{1b})₁₋₄, —(CR^{1a}R^{1b})₁₋₄NR^{1c}(CR^{1a}R^{1b})₁₋₄, optionally substituted 3-11 membered cycloalkyl, 3-11 membered heterocyclyl, aryl, and heteroaryl;
wherein R^{1a} and R^{1b} are each independently selected from the group consisting of —H, D, -halo, —C₁-C₈alkyl, —O—C₁-C₈alkyl, —C₁-C₆haloalkyl, —S—C₁-C₈alkyl, μ-NHC₁-C₈alkyl, —N(C₁-C₈alkyl)₂, 3-11 membered cycloalkyl, aryl, heteroaryl, 3-11 membered heterocyclyl, —O-(3-11 membered cycloalkyl), —S-(3-11 membered cycloalkyl), NH-(3-11 membered cycloalkyl), N(3-11 membered cycloalkyl)₂, N-(3-11 membered cycloalkyl)(C₁-C₈alkyl), —OH, —NH₂, —SH, —SO₂C₁-C₈alkyl, —SO₂-aryl, —SO₂-heteroaryl, SO(NH)C₁-C₈alkyl, P(O)(OC₁-C₈alkyl)(C₁-C₈alkyl), —P(O)(OC₁-C₈alkyl)₂, —C≡C—C₁-C₈alkyl, —C≡CH, —CH═CH(C₁-C₈alkyl), —C(C₁-C₈alkyl)═CH(C₁-C₈alkyl), —C(C₁-C₈alkyl)═C(C₁-C₈alkyl)₂, —Si(OH)₃, —Si(C₁-C₈alkyl)₃, —Si(OH)(C₁-C₈alkyl)₂, —C(O)C₁-C₈alkyl, —C(O)OC₁-C₈alkyl, —CO₂H, —CN, —NO₂, —SF₅, —SO₂NHC₁-C₈alkyl, —SO₂N(C₁-C₈alkyl)₂, —SO(NH)NHC₁-C₈alkyl, —SO(NH)N(C₁-C₈alkyl)₂, —SONHC₁-C₈alkyl, —SON(C₁-C₈alkyl)₂, —CONHC₁-C₈alkyl, —CON(C₁-C₈alkyl)₂, —N(C₁-C₈alkyl)CONH(C₁-C₈alkyl), —N(C₁-C₈alkyl)CON(C₁-C₈alkyl)₂, —NHCONH(C₁-C₈alkyl), —NHCON(C₁-C₈alkyl)₂, —NHCONH₂, —N(C₁-C₈alkyl)SO₂NH(C₁-C₈alkyl), —N(C₁-C₈alkyl)SO₂N(C₁-C₈alkyl)₂, —NHSO₂NH(C₁-C₈alkyl), —NHSO₂N(C₁-C₈alkyl)₂, or —NHSO₂NH₂; and
R^{1c} and R^{1d} are each independently selected from the group consisting of H, D, optionally substituted C₁₋₄ alkyl, C₃₋₈ cyclcoalkyl, C₃₋₈ heterocyclcoalkyl, aryl, or heteroaryl.

In other embodiments, q=2 and Y in Formula IA is a chemical moiety represented by the formula: -A₁-A₂-, wherein each of A₁₋₂ is independently selected from the group consisting of O, S, SO, SO₂, NR^{1c}, SO₂NR^{1c}SONR^{1c}, SO(═NR^{1c}) SO(═NR^{1c})NR^{1d}, CONR¹, NR^{1c}CONR^{1d}, NR^{1c}C(O)O, NR^{1c}SO₂NR^{1d}, CO, CR^{1a}═CR^{1b}, C≡C, SiR^{1a}R^{1b}, P(O)R^{1a}, P(O)OR^{1a}, (CR^{1a}R^{1b})₁₋₄, —(CR^{1a}R^{1b})₁₋₄O(CR^{1a}R^{1b})₁₋₄, —(CR^{1a}R^{1b})₁₋₄S(CR^{1a}R^{1b})₁₋₄, —(CR^{1a}R^{1b})₁₋₄NR^{1c}(CR^{1a}R^{1b})₁₋₄, optionally substituted 3-11 membered cycloalkyl, 3-11 membered heterocyclyl, aryl, and heteroaryl;
wherein R^{1a} and R^{1b} are each independently selected from the group consisting of —H, D, -halo, —C₁-C₈alkyl, —O—C₁-C₈alkyl, —C₁-C₆haloalkyl, —S—C₁-C₈alkyl, —NHC₁-C₈alkyl, —N(C₁-C₈alkyl)₂, 3-11 membered cycloalkyl, aryl, heteroaryl, 3-11 membered heterocyclyl, —O-(3-11 membered cycloalkyl), —S-(3-11 membered cycloalkyl), NH-(3-11 membered cycloalkyl), N(3-11 membered cycloalkyl)₂, N-(3-11 membered cycloalkyl)(C₁-C₈alkyl), —OH, —NH₂, —SH, —SO₂C₁-C₈alkyl, —SO₂-aryl, —SO₂-heteroaryl, SO(NH)C₁-C₈alkyl, P(O)(OC₁-C₈alkyl)(C₁-C₈alkyl), —P(O)(OC₁-C₈alkyl)₂, —C≡C—C₁-C₈alkyl, —C≡CH, —CH═CH(C₁-C₈alkyl), —C(C₁-C₈alkyl)═CH(C₁-C₈alkyl), —C(C₁-C₈alkyl)═C(C₁-C₈alkyl)₂, —Si(OH)₃, —Si(C₁-C₈alkyl)₃, —Si(OH)(C₁-C₈alkyl)₂, —C(O)C₁-C₈alkyl, —C(O)OC₁-C₈alkyl, —CO₂H, —CN, —NO₂, —SF₅, —SO₂NHC₁-C₈alkyl, —SO₂N(C₁-C₈alkyl)₂, —SO(NH)NHC₁-C₈alkyl, —SO(NH)N(C₁-C₈alkyl)₂, —SONHC₁-C₈alkyl, —SON(C₁-C₈alkyl)₂, —CONHC₁-C₈alkyl, —CON(C₁-C₈alkyl)₂, —N(C₁-C₈alkyl)CONH(C₁-C₈alkyl), —N(C₁-C₈alkyl)CON(C₁-C₈alkyl)₂, —NHCONH(C₁-C₈alkyl), —NHCON(C₁-C₈alkyl)₂, —NHCONH₂, —N(C₁-C₈alkyl)SO₂NH(C₁-C₈alkyl), —N(C₁-C₈alkyl)SO₂N(C₁-C₈alkyl)₂, —NHSO₂NH(C₁-C₈alkyl), —NHSO₂N(C₁-C₈alkyl)₂, or —NHSO₂NH₂; and
R^{1c} and R^{1d} are each independently selected from the group consisting of H, D, optionally substituted C₁₋₄ alkyl, C₃₋₈ cyclcoalkyl, C₃₋₈ heterocyclcoalkyl, aryl, or heteroaryl.

In other embodiments, q=1 and Y in Formula IA is a chemical moiety represented by the formula: -A₁-, wherein A₁ is selected from the group consisting of O, S, SO, SO₂, NR^{1c}, SO₂NR^{1c}, SONR^{1c}, SO(═NR^{1c}), SO(═NR^{1c})NR^{1d}, CONR^{1c}, NR^{1c}CONR^{1d}, NR^{1c}C(O)O, NR^{1c}SO₂NR^{1d}, CO, CR^{1a}═CR^{1b}, C≡C, SR^{1a}R^{1b}, P(O)R^{1a}, P(O)OR^{1a}, (CR^{1a}R^{1b})₁₋₄, —(CR^{1a}R^{1b})₁₋₄O(CR^{1a}R^{1b})₁₋₄, —(CR^{1a}R^{1b})₁₋₄S(CR^{1a}R^{1b})₁₋₄, —(CR^{1a}R^{1b})₁₋₄NR^{1c}(CR^{1a}R^{1b})₁₋₄, optionally substituted 3-11 membered cycloalkyl, 3-11 membered heterocyclyl, aryl, and heteroaryl; wherein R^{1a} and R^{1b} are each independently selected from the group consisting of —H, D, -halo, —C₁-C₈alkyl, —O—C₁-C₈alkyl, —C₁-C₆haloalkyl, —S—C₁-C₈alkyl, —NHC₁-C₈alkyl, —N(C₁-C₈alkyl)₂, 3-11 membered cycloalkyl, aryl, heteroaryl, 3-11 membered heterocyclyl, —O-(3-11 membered cycloalkyl), —S-(3-11 membered cycloalkyl), NH-(3-11 membered cycloalkyl), N(3-11 membered cycloalkyl)₂, N-(3-11 membered cycloalkyl)(C₁-C₈alkyl), —OH, —NH₂, —SH, —SO₂C₁-C₈alkyl, —SO₂-aryl, SO(NH)C₁-C₈alkyl, P(O)(OC₁-C₈alkyl)(C₁-C₈alkyl), —P(O)(OC₁-C₈alkyl)₂, —C≡C—C₁-C₈alkyl, —C≡CH, —CH═CH(C₁-C₈alkyl), —C(C₁-C₈alkyl)═CH(C₁-C₈alkyl), —C(C₁-C₈alkyl)═C(C₁-C₈alkyl)₂, —Si(OH)₃, —Si(C₁-C₈alkyl)₃, —Si(OH)(C₁-C₈alkyl)₂, —C(O)C₁-C₈alkyl, —C(O)OC₁-C₈alkyl, —CO₂H, —CN, —NO₂, —SF₅, —SO₂NHC₁-C₈alkyl, —SO₂N(C₁-C₈alkyl)₂, —SO(NH)NHC₁-C₈alkyl, —SO(NH)N(C₁-C₈alkyl)₂, —SONHC₁-C₈alkyl, —SON(C₁-C₈alkyl)₂, —CONHC₁-C₈alkyl, —CON(C₁-C₈alkyl)₂, —N(C₁-C₈alkyl)CONH(C₁-C₈alkyl), —N(C₁-C₈alkyl)CON(C₁-C₈alkyl)₂, —NHCONH(C₁-C₈alkyl), —NHCON(C₁-C₈alkyl)₂, —NHCONH₂, —N(C₁-C₈alkyl)SO₂NH(C₁-C₈alkyl), —N(C₁-C₈alkyl)SO₂N(C₁-C₈alkyl)₂, —NHSO₂NH(C₁-C₈alkyl), —NHSO₂N(C₁-C₈alkyl)₂, or —NHSO₂NH₂; and
R^{1c} and R^{1d} are each independently selected from the group consisting of H, D, optionally substituted C₁₋₄ alkyl, C₃₋₈ cyclcoalkyl, C₃₋₈ heterocyclcoalkyl, aryl, or heteroaryl.

In some embodiments, Y in Formula IA is a covalent bond, 3-11 membered cycloalkyl optionally substituted with 1-6 R^{1a} or R^{1b} groups, 3-11 membered heterocyclyl optionally substituted with 1-6 R^{1a} or R^{1b} groups, —(CR^{1a}R^{1b})₁₋₅, —(CR^{1a}═CR^{1b})—, —(CR^{1a}R^{1b})₁₋₅-A- wherein A is O, S, or NR^{1c}—(CR^{1a}R^{1b})₁₋₅-A-(CR^{1a}R^{1b})₁₋₅— wherein A is O, S, or NR^{1c}, —(CR^{1a}R^{1b})₁₋₅-A-(CR^{1a}R^{1b})₁₋₅-A- wherein A is O, S, or NR^{1c}—(CR^{1a}R^{1b})₁₋₅—(CR^{1a}═CR^{1b})—(CR^{1a}R^{1b})₁₋₅—, —(CR^{1a}R^{1b})₁₋₅—(CR^{1a}═CR^{1b})—(CR^{1a}R^{1b})₁₋₅-A- wherein A is O, S, or NR^{1c}, —(CR^{1a}R^{1b})₁₋₅—(C≡C)—(CR^{1a}R^{1b})₁₋₅—, —(CR^{1a}R^{1b})₁₋₅—(C≡C)—(CR^{1a}R^{1b})₁₋₅-A- wherein A is O, S, or NR^{1c}, —(C≡C)—(CR^{1a}R^{1b})₁₋₅-A-(CR^{1a}R^{1b})₁₋₅— wherein A is O, S, or NR^{1c}, —(C≡C)—(CR^{1a}R^{1b})₁₋₅, —(CR^{1a}R^{1b})₁₋₅-(3-11 membered cycloalkyl optionally substituted with 1-6 R^{1a} or R^{1b} groups)-, —(CR^{1a}R^{1b})₁₋₅-(3-11 membered heterocyclyl optionally substituted with 1-6 R^{1a} or R^{1b} groups)-, -(3-11 membered cycloalkyl optionally substituted with 1-6 R^{1a} or R^{1b} groups)-(CR^{1a}R^{1b})₁₋₅—, -(3-11 membered heterocyclyl optionally substituted with 1-6 R^{1a} or R^{1b} groups) —(CR^{1a}R^{1b})₁₋₅—, —(CR^{1a}R^{1b})₁₋₅-(3-11 membered cycloalkyl optionally substituted with 1-6 R^{1a} or R^{1b} groups)-A-, —(CR^{1a}R^{1b})₁₋₅-(3-11 membered heterocyclyl optionally substituted with 1-6 R^{1a} or R^{1b} groups)-A-, —(CR^{1a}R^{1b})₁₋₅-(3-11 membered cycloalkyl optionally substituted with 1-6 R^{1a} or R^{1b} groups)-(CR^{1a}R^{1b})₁₋₅, —(CR^{1a}R^{1b})₁₋₅-(3-11 membered cycloalkyl optionally substituted with 1-6 R^{1a} or R^{1b} groups)-A- wherein A is O, S, or NR^{1c}, —(CR^{1a}R^{1b})₁₋₅-(3-11 membered cycloalkyl optionally substituted with 1-6 R^{1a} or R^{1b} groups)-(CR^{1a}R^{1b})₁₋₅-A- wherein A is O, S, or NR^{1c}—(CR^{1a}R^{1b})₁₋₅-A-(3-11 membered cycloalkyl optionally substituted with 1-6 R^{1a} or R^{1b} groups)- wherein A is O, S, or NR^{1c}—(CR^{1a}R^{1b})₁₋₅-(3-11 membered heterocyclyl optionally substituted with 1-6 R^{1a} or R^{1b} groups)-(CR^{1a}R^{1b})₁₋₅, —(CR^{1a}R^{1b})₁₋₅-(3-11 membered heterocyclyl optionally substituted with 1-6 R^{1a} or R^{1b} groups)-(CR^{1a}R^{1b})₁₋₅-A- wherein A is O, S, or NR^{1c}—(CR^{1a}R^{1b})₁₋₅-(3-11 membered heterocyclyl optionally substituted with 1-6 R^{1a} or R^{1b} groups)-A- wherein A is O, S, or NR^{1c}—(CR^{1a}R^{1b})₁₋₅-A-(3-11 membered heterocyclyl optionally substituted with 1-6 R^{1a} or R^{1b} groups)- wherein A is O, S, or NR^{1c}—(CR^{1a}R^{1b})₅-(3-11 membered cycloalkyl optionally substituted with 1-6 R^{1a} or R^{1b} groups)-(CR^{1a}R^{1b})₁₋₅-A- wherein A is O, S, or NR^{1c}—(CR^{1a}R^{1b})₁₋₅-A-(3-11 membered cycloalkyl optionally substituted with 1-6 R^{1a} or R^{1b} groups)- wherein A is O, S, or NR^{1c}—(CR^{1a}R^{1b})₁₋₅-A-(3-11 membered cycloalkyl optionally substituted with 1-6 R^{1a} or R^{1b} groups)-(CR^{1a}R^{1b})₁₋₅-A- wherein each A is independently O, S, or NR^{1c}, —(CR^{1a}R^{1b})₁₋₅-A-(3-11 membered heterocyclyl optionally substituted with 1-6 R^{1a} or R^{1b} groups)-(CR^{1a}R^{1b})₁₋₅-A- wherein each A is independently O, S, or NR^{1c}—(CR^{1a}R^{1b})₁₋₅-A-(CR^{1a}R^{1b})₁₋₅-A- wherein A is O, S, or NR^{1c}, —(CR^{1a}R^{1b})₁₋₅-A-(CR^{1a}R^{1b})₁₋₅-A-(CR^{1a}R^{1b})₁₋₅-A-(CR^{1a}R^{1b})₁₋₅-A- wherein A is O, S, or NR^{1c}, —(CR^{1a}R^{1b})₁₋₅-A-(CO) wherein A is O, S, or NR^{1c}, —(CR^{1a}R^{1b})₁₋₅—(CR^{1a}═CR^{1b})—(CR^{1a}R^{1b})₁₋₅-A-(CO)— wherein A is O, S, or NR^{1c}—(CR^{1a}R^{1b})₁₋₅—(C≡C)—(CR^{1a}R^{1b})₁₋₅-A-(CO)— wherein A is O, S, or NR^{1c}, —(CR^{1a}R^{1b})₁₋₅-(3-11 membered cycloalkyl optionally substituted with 1-6 R^{1a} or R^{1b} groups)-(CR^{1a}R^{1b})₁₋₅-A-(CO)— wherein A is O, S, or NR^{1c}—(CR^{1a}R^{1b})₁₋₅-A-(CO)-(3-11 membered cycloalkyl optionally substituted with 1-6 R^{1a} or R^{1b} groups)- wherein A is O, S, or NR^{1c}, —(CR^{1a}R^{1b})₁₋₅-(3-11 membered heterocyclyl optionally substituted with 1-6 R^{1a} or R^{1b} groups)-(CR^{1a}R^{1b})₁₋₅-A-(CO)— wherein A is O, S, or NR^{1c}—(CR^{1a}R^{1b})₁₋₅-A-(CO)-(3-11 membered heterocyclyl optionally substituted with 1-6 R^{1a} or R^{1b} groups)- wherein A is O, S, or NR^{1c}—(CR^{1a}R^{1b})₁₋₅-A-(3-11 membered cycloalkyl optionally substituted with 1-6 R^{1a} or R^{1b} groups)-A-(CO)— wherein each A is independently O, S, or NR^{1c}, -(3-11 membered cycloalkyl optionally substituted with 1-6 R^{1a} or R^{1b} groups)-CO—(CR^{1a}R^{1b})₁₋₅-A- wherein A is O, S, or NR^{1c}—(CR^{1a}R^{1b})₁₋₅-(3-11 membered cycloalkyl optionally substituted with 1-6 R^{1a} or R^{1b} groups)-(CR^{1a}R^{1b})₁₋₅-A-(CO)— wherein A is O, S, or NR^{1c}—(CR^{1a}R^{1b})₁₋₅-(3-11 membered heterocyclyl optionally substituted with 1-6 R^{1a} or R^{1b} groups)-(CR^{1a}R^{1b})₁₋₅-A-(CO)— wherein A is O, S, or NR^{1c}, -(3-11 membered cycloalkyl optionally substituted with 1-6 R^{1a} or R^{1b} groups)-(CR^{1a}R^{1b})₁₋₅—, or -(3-11 membered heterocyclyl optionally substituted with 1-6 R^{1a} or R^{1b} groups)-(CR^{1a}R^{1b})₁₋₅.

In some embodiments, Y in Formula IA is —CR^{1a}═CR^{1b}—, such as, for example, —CH═CH—.

In some embodiments, Y in Formula IA is —(CR^{1a}R^{1b})₁₋₅, for example —(CH₂)₁₋₅—, —CH₂—, —CH₂CH₂CH₂— and the like.

In some embodiments, Y in Formula IA is —(CR^{1a}R^{1b})₁₋₅-A- wherein A is O, S, or NR^{c}, such as for example, —(CH₂)₁₋₅—O—, —(CH₂)₁₋₅—S—, —(CH₂)₁₋₅—NH—, or —(CH₂)₀₋₂—(C(CH₃)₂)—(CH₂)₀₋₂—O—.

In other embodiments, Y in Formula IA is —(CR^{1a}R^{1b})₁₋₅-A-(CR^{1a}R^{1b})₁₋₅— wherein A is O, S, or NR^{1c}, such as, for example, —(CH₂)₁₋₅—O—(CH₂)₁₋₅—, —(CH₂)₁₋₅—S—(CH₂)₁₋₅—, —(CH₂)₁₋₅—NH—(CH₂)₁₋₅—.

In some embodiments, Y in Formula IA is —(C≡C)—(CR^{1a}R^{1b})₁₋₅, such as, for example, —(C≡C)—(CH₂)₂—, and the like.

In some embodiments, Y in Formula IA is —(CR^{1a}R^{1b})₁₋₅-(3-11 membered cycloalkyl optionally substituted with 1-6 R^{1a} or R^{1b} groups)-, such as, for example, —CH₂-cyclobutyl-.

In some embodiments, Y in Formula IA is —(CR^{1a}R^{1b})₁₋₅-(3-11 membered cycloalkyl optionally substituted with 1-6 R^{1a} or R^{1b} groups)-(CR^{1a}R^{1b})₁₋₅, such as, for example, —CH₂— cyclobutyl-CH₂— and the like.

In some embodiments, Y in Formula IA is —(CR^{1a}R^{1b})₁₋₅-(3-11 membered heterocyclyl optionally substituted with 1-6 R^{1a} or R^{1b} groups)-(CR^{1a}R^{1b})₁₋₅, such as, for example, —CH₂-azetidinyl-CH₂—.

In some embodiments, Y in Formula IA is —(CR^{1a}R^{1b})₁₋₅-(3-11 membered heterocyclyl optionally substituted with 1-6 R^{1a} or R^{1b} groups)-, such as, for example, —CH₂— azetidinyl-.

In some embodiments, Y in Formula IA is -(3-11 membered heterocyclyl optionally substituted with 1-6 R^{1a} or R^{1b} groups) —(CR^{1a}R^{1b})₁₋₅—, such as, for example, -azetidinyl-CH₂—, -pyrolidnyl-CH₂—, -piperidinyl-CH₂—, and the like.

In some embodiments, Y in Formula IA is —(CR^{1a}R^{1b})₁₋₅-(3-11 membered cycloalkyl optionally substituted with 1-6 R^{1a} or R^{1b} groups)-(CR^{1a}R^{1b})₁₋₅-A- wherein A is O, S, or NR^{1c} such as, for example, —CH₂-cyclopropyl-CH₂—O—, and the like.

In some embodiments, Y in Formula IA is —(CR^{1a}R^{1b})₁₋₅-(3-11 membered heterocyclyl optionally substituted with 1-6 R^{1a} or R^{1b} groups)-(CR^{1a}R^{1b})₁₋₅-A- wherein A is O, S, or NR^{1c}, such as, for example, —CH₂-piperidinyl-CH₂CH₂—O—, and the like.

In some embodiments, Y in Formula IA is —(CR^{1a}R^{1b})₁₋₅-(3-11 membered heterocyclyl optionally substituted with 1-6 R^{1a} or R^{1b} groups)-A- wherein A is O, S, or NR^{1c} such as, for example, —CH₂-azetidinyl-O—, and the like.

In some embodiments, Y in Formula IA is —(CR^{1a}R^{1b})₁₋₅-A-(3-11 membered heterocyclyl optionally substituted with 1-6 R^{1a} or R^{1b} groups)- wherein A is O, S, or NR^{1c} such as, for example, —CH₂—O-azetidinyl-, —CH₂—NH-azetidinyl-, and the like.

In other embodiments, Y in Formula IA is —(CR^{1a}R^{1b})₁₋₅-A-(3-11 membered cycloalkyl optionally substituted with 1-6 R^{1a} or R^{1b} groups)- wherein A is O, S, or NR^{c}, such as —CH₂—O-cyclobutylene-, —CH₂—NH-cyclobutylene-, and the like.

In some embodiments, Y in Formula IA is —(CR^{1a}R^{1b})₁₋₅-A-(CR^{1a}R^{1b})₁₋₅-A- wherein A is O, S, or NR^{c}, such as, for example, —CH₂—O—CH₂CH₂—O—.

In some embodiments, Y in Formula IA is

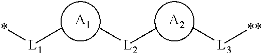

wherein
** is a point of attachment to PTM;
L₁, L₂, and L₃ are each independently a bond, CR^{1a}R^{1b}, O, S, SO, SO₂, NRI° SO₂NR^{1c}, SONR^{1c}, SO(═NR^{1c}), SO(═NR^{1c})NR^{1d}, CONR^{1c}, NR^{1c}CONR^{1d}, NR^{1c}C(O)O, NR^{1c}SO₂NR^{1d}, CO, CR^{1a}═CR^{1b}, C≡C, SiR^{1a}R^{1b}, P(O)R^{1a}, P(O)OR^{1a}, (CR^{1a}R^{1b})₁₋₄, —(CR^{1a}R^{1b})₁₋₄O(CR^{1a}R^{1b})₁₋₄, —(CR^{1a}R^{1b})₁₋₄S(CR^{1a}R^{1b})₁₋₄, —(CR^{1a}R^{1b})₁₋₄NR^{1c}(CR^{1a}R^{1b})₁, —NR^{1c}C(═NCN)NR^{1d}, NR^{1c}C(═NCN), NR^{1c}C(═CNO₂)NR^{1d};
ring A₁ and ring A₂ are each independently 3-12 membered cycloalkyl, optionally substituted with 1-6 R^{1a} or R^{1b} groups, 3-12 membered heterocyclyl optionally substituted with 1-6 R^{1a} or R^{1b} groups, aryl optionally substituted with 1-6 R^{1a} or R^{1b} groups, or heteroaryl optionally substituted with 1-6 R^{1a} or R^{1b} groups,
wherein R^{1a}, R^{1b}, R^{1c}, R^{1d} and R^{1e} are each independently, —H, D, -halo, —C₁-C₈alkyl, —O—C₁-C₈alkyl, —C₁-C₆haloalkyl, —S—C₁-C₈alkyl, —NHC₁-C₈alkyl, —N(C₁-C₈alkyl)₂, 3-11 membered cycloalkyl, aryl, heteroaryl, 3-11 membered heterocyclyl, —O-(3-11 membered cycloalkyl), —S-(3-11 membered cycloalkyl), NH-(3-11 membered cycloalkyl), N(3-11 membered cycloalkyl)₂, N-(3-11 membered cycloalkyl)(C₁-C₈alkyl), —OH, —NH₂, —SH, —SO₂C₁-C₈alkyl, —SO₂-aryl, —SO₂-heteroaryl, SO(NH)C₁-C₈alkyl, P(O)(OC₁-C₈alkyl)(C₁-C₈alkyl), —P(O)(OC₁-C₈alkyl)₂, —C≡C—C₁-C₈alkyl, —C≡CH, —CH═CH(C₁-C₈alkyl), —C(C₁-C₈alkyl)═CH(C₁-C₈alkyl), —C(C₁-C₈alkyl)═C(C₁-C₈alkyl)₂, —Si(OH)₃, —Si(C₁-C₈alkyl)₃, —Si(OH)(C₁-C₈alkyl)₂, —C(O)C₁-C₈alkyl, —C(O)OC₁-C₈alkyl, —CO₂H, —CN, —CF₃, —CHF₂, —CH₂F, —NO₂, —SF₅, —SO₂NHC₁-C₈alkyl, —SO₂N(C₁-C₈alkyl)₂, —SO(NH)NHC₁-C₈alkyl, —SO(NH)N(C₁-C₈alkyl)₂, —SONHC₁-C₈alkyl, —SON(C₁-C₈alkyl)₂, —CONHC₁-C₈alkyl, —CON(C₁-C₈alkyl)₂, —N(C₁-C₈alkyl)CONH(C₁-C₈alkyl), —N(C₁-C₈alkyl)CON(C₁-C₈alkyl)₂, —NHCONH(C₁-C₈alkyl), —NHCON(C₁-C₈alkyl)₂, —NHCONH₂, —N(C₁-C₈alkyl)SO₂NH(C₁-C₈alkyl), —N(C₁-C₈alkyl)SO₂N(C₁-C₈alkyl)₂, —NHSO₂NH(C₁-C₈alkyl), —NHSO₂N(C₁-C₈alkyl)₂, or —NHSO₂NH₂; and where R^{1a} or R^{1b}, each independently may be optionally linked to other groups to form cycloalkyl and/or heterocyclyl moiety, optionally substituted with 0-4 R^{1e} groups.

In some embodiments, Y in Formula IA is

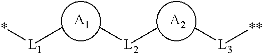

wherein
** is a point of attachment to PTM;
L₁ is a bond, (C(R¹⁰)₂)ₚ or CO;
L₂ is a bond, (C(R¹⁰)₂)ₚ or CO;
L₃ is a bond, (C(R¹⁰)₂)ₚ or CO;
each p is independently 1, 2, 3, or 4;
each R¹⁰ is independently H, D, or C₁-C₄ alkyl;
ring A₁ is a 3-7 membered cycloalkyl group, a 4-10-membered heterocycloalkyl group, an aryl group, or a heteroaryl group; and
ring A₂ is a 3-7 membered cycloalkyl group, a 4-12-membered heterocycloalkyl group, an aryl group, or a heteroaryl group.

According to the disclosure, L₁ is a bond, (C(R¹⁰)₂)ₚ or CO. In some embodiments, L₁ is a bond. In some embodiments, L₁ is (C(R¹⁰)₂)ₚ. In other embodiments, L₁ is CO.

According to the disclosure, L₂ is a bond, (C(R¹⁰)₂)ₚ or CO. In some embodiments, L₂ is a bond. In some embodiments, L₂ is (C(R¹⁰)₂)ₚ. In other embodiments, L₂ is CO.

According to the disclosure, L₃ is a bond, (C(R¹⁰)₂)ₚ or CO. In some embodiments, L₃ is a bond. In some embodiments, L₃ is (C(R¹⁰)₂)ₚ. In other embodiments, L₃ is CO.

According to the disclosure, each p is independently 1, 2, 3, or 4. In some embodiments, each p is 1. In some embodiments, at least one p is 1. In some embodiments, each p is 2. In some embodiments, at least one p is 2. In other embodiments, each p is 3. In other embodiments, at least one p is 3. In other embodiments, each p is 4. In other embodiments, at least one p is 4.

According to the disclosure, each R¹⁰ is independently H, D, or C₁-C₄ alkyl. In some embodiments, each R¹⁰ is H. In some embodiments, at least one R¹⁰ is H. In some embodiments, each R¹⁰ is D. In some embodiments, at least one R¹⁰ is D. In other embodiments, each R¹⁰ is C₁-C₄ alkyl. In other embodiments, at least one R¹⁰ is C₁-C₄ alkyl. In other embodiments, each R¹⁰ is methyl or ethyl. In other embodiments, at least one R¹⁰ is methyl or ethyl.

According to the disclosure, ring A₁ is a 3-7 membered cycloalkyl group, a 4-10-membered heterocycloalkyl group, an aryl group, or a heteroaryl group. In some embodiments, ring A₁ is a 3-7 membered cycloalkyl group. In some embodiments, ring A₁ is a 4-10-membered heterocycloalkyl group. In other embodiments, ring A₁ is an aryl group. In other embodiments, ring A₁ is a heteroaryl group.

In some embodiments, ring A₁ is a piperazine group, a morpholine group, a piperidine group, a pyrrolidine group, an azetidine group or an azabicyclo-alkyl group. In some embodiments, ring A₁ is a piperazine group. In some embodiments, ring A₁ is a morpholine group. In other embodiments, ring A₁ is a piperidine group. In other embodiments, ring A₁ is a pyrrolidine group. In yet other embodiments, ring A₁ is an azetidine group. In yet other embodiments, ring A₁ is an azabicyclo-alkyl group.

In other embodiments, ring A₁ is a piperidine or pyrrolidine group.

According to the disclosure, ring A₂ is a 3-7 membered cycloalkyl group, a 4-12-membered heterocycloalkyl group, an aryl group, or a heteroaryl group. In some embodiments, ring A₂ is a 3-7 membered cycloalkyl group. In some embodiments, ring A₂ is a 4-12-membered heterocycloalkyl group. In other embodiments, ring A₂ is an aryl group. In other embodiments, ring A₂ is a heteroaryl group.

In some embodiments, ring A₂ is a piperazine group, a piperidine group, a pyrrolidine group, an azaspiroalkyl group, a diazaspiroalkyl group or an azabicycloalkyl group. In some embodiments, ring A₂ is a piperazine group. In some embodiments, ring A₂ is a piperidine group. In other embodiments, ring A₂ is a pyrrolidine group. In other embodiments, ring A₂ is an azaspiroalkyl group. In yet other embodiments, ring A₂ is a diazaspiroalkyl group. In yet other embodiments, ring A₂ is an azabicycloalkyl group.

In other embodiments, ring A₂ is a piperazine group, an azaspiroalkyl group or a diazaspiroalkyl group.

In other embodiments, ring A₂ is aryl optionally substituted with 1-6 R^{1a} or R^{1b} groups.

In some embodiments, Y in Formula IA is

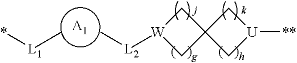

wherein
* is a point of attachment to ULM;
** is a point of attachment to PTM;
W is CR₁₁ or N;
U is CR₁₁ or N;
R¹¹ is H, D, F, C₁₋₆alkyl, or C₁₋₆alkoxide; and
each g, h, j and k is independently 0, 1 or 2.

According to the disclosure, W is CR₁₁ or N. In some embodiments, W is N. In other embodiments, W is CR₁₁.

According to the disclosure, U is CR₁₁ or N. In some embodiments, U is N. In other embodiments, U is CR₁₁.

In some embodiments, W and U are N. In other embodiments, W is CR₁₁ and U are N. In yet other embodiments, W is N and U is CR₁₁.

According to the disclosure, R₁ is H, D, F, C₁₋₆alkyl, or C₁₋₆alkoxide. In some embodiments, R₁₁ is H. In other embodiments, R₁₁ is D. In other embodiments, R₁₁ is F. In other embodiments, R₁₁ is C₁₋₆alkyl. In other embodiments, R₁₁ is C₁₋₆alkoxide.

According to the disclosure, g is 0, 1 or 2. In some embodiments, g is 0. In other embodiments, g is 1. In yet other embodiments, g is 2.

According to the disclosure, h is 0, 1 or 2. In some embodiments, h is 0. In other embodiments, h is 1. In yet other embodiments, h is 2.

According to the disclosure, j is 0, 1 or 2. In some embodiments, j is 0. In other embodiments, j is 1. In yet other embodiments, j is 2.

According to the disclosure, k is 0, 1 or 2. In some embodiments, k is 0. In other embodiments, k is 1. In yet other embodiments, k is 2.

In some embodiments, j and g are each 2. In some embodiments, k and h are each 1. In other embodiments, j and g are each 1. In yet other embodiments, k and h are each 2.

In some embodiments, Y in Formula IA is

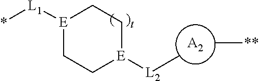

wherein
* is a point of attachment to ULM;
** is a point of attachment to PTM;
each E is independently CR₁₁ or N;
R₁₁ is H, F, C₁₋₆alkyl, or C₁₋₆alkoxide; and
t is 0, 1, 2 or 3.

According to the disclosure, each E is independently CR₁₁ or N. In some embodiments, each E is N. In other embodiments, one E is CR₁₁ and the other E is N. In yet other embodiments, each E is CR₁₁.

According to the disclosure, t is 0, 1, 2 or 3. In some embodiments, t is 0. In some embodiments, t is 1. In other embodiments, t is 2. In other embodiments, t is 3.

According to the disclosure, R₁₁ is H, D, F, C₁₋₆alkyl, or C₁₋₆alkoxide. In some embodiments, R₁₁ is H. In other embodiments, R₁₁ is D. In other embodiments, R₁₁ is F. In other embodiments, R₁₁ is C₁₋₆alkyl. In other embodiments, R₁₁ is C₁₋₆alkoxide.

In some embodiments, Y in Formula IA is

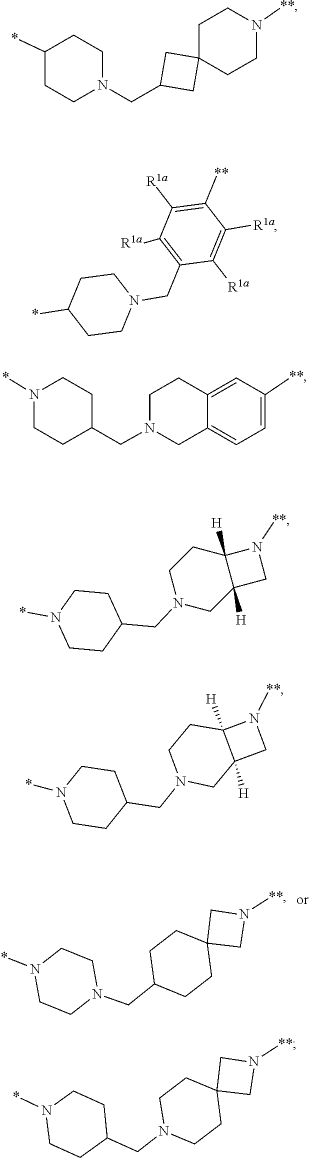

wherein * is a point of attachment to ULM and ** is a point of attachment to PTM.

In some embodiments, Y in Formula IA is

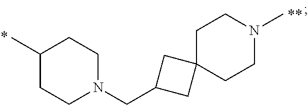

wherein * is a point of attachment to ULM and ** is a point of attachment to PTM.

In some embodiments, Y in Formula IA is

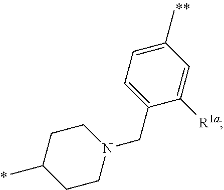

wherein * is a point of attachment to ULM and ** is a point of attachment to PTM.

In other embodiments, Y in Formula IA is

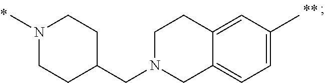

wherein * is a point of attachment to ULM and ** is a point of attachment to PTM.

In other embodiments, Y in Formula IA is

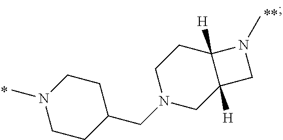

wherein * is a point of attachment to ULM and ** is a point of attachment to PTM.

In other embodiments, Y in Formula IA is

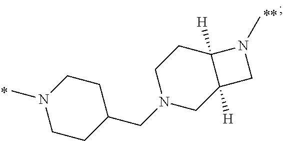

wherein * is a point of attachment to ULM and ** is a point of attachment to PTM.

In yet other embodiments, Y in Formula IA is

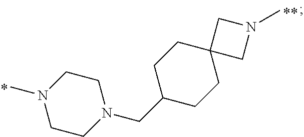

wherein * is a point of attachment to ULM and ** is a point of attachment to PTM.

In yet other embodiments, Y in Formula IA is

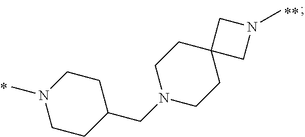

wherein * is a point of attachment to ULM and ** is a point of attachment to PTM.

According to the disclosure, Y-PTM is a compound of formula IA-1

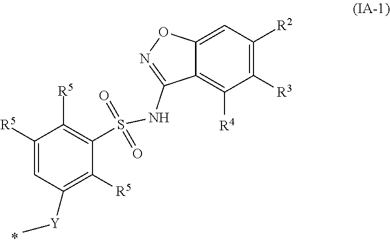

or a pharmaceutically acceptable salt thereof, wherein R², R³, R⁴, R⁵ and Y are as defined herein.

According to the disclosure, Y-PTM is a compound of formula IA-2

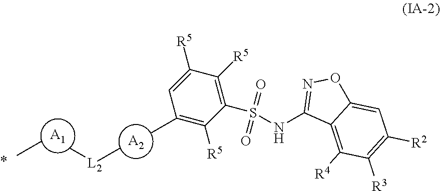

or a pharmaceutically acceptable salt thereof, wherein R², R³, R⁴, R⁵, L₂, ring A₁ and ring A₂ are as defined herein.

According to the disclosure, ring A₁ is selected from:

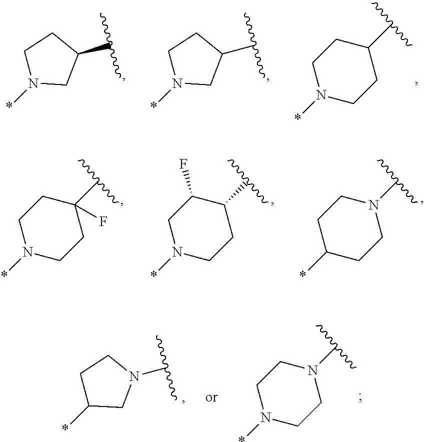

wherein [symbol] is a point of attachment to L₂ and * is a point of attachment to ULM.

[symbol]

In some embodiments, ring A₁ is

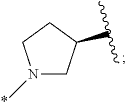

wherein [symbol] is a point of attachment to L₂ and * is a point of attachment to ULM.

In some embodiments, ring A₁ is

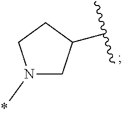

wherein [symbol] is a point of attachment to L₂ and * is a point of attachment to ULM.

In some embodiments, ring A₁ is

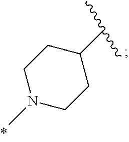

wherein [symbol] is a point of attachment to L₂ and * is a point of attachment to ULM.

In other embodiments, ring A₁ is

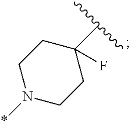

wherein [symbol] is a point of attachment to L₂ and * is a point of attachment to ULM.

In other embodiments, ring A₁ is

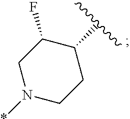

wherein [symbol] is a point of attachment to L₂ and * is a point of attachment to ULM.

In other embodiments, ring A₁ is

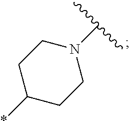

wherein [symbol] is a point of attachment to L₂ and * is a point of attachment to ULM.

In yet other embodiments, ring A₁ is

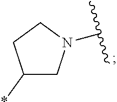

wherein [symbol] is a point of attachment to L₂ and * is a point of attachment to ULM.

In yet other embodiments, ring A₁ is

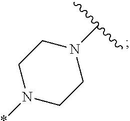

wherein [symbol] is a point of attachment to L₂ and * is a point of attachment to ULM.

According to the disclosure, ring A₂ is selected from:

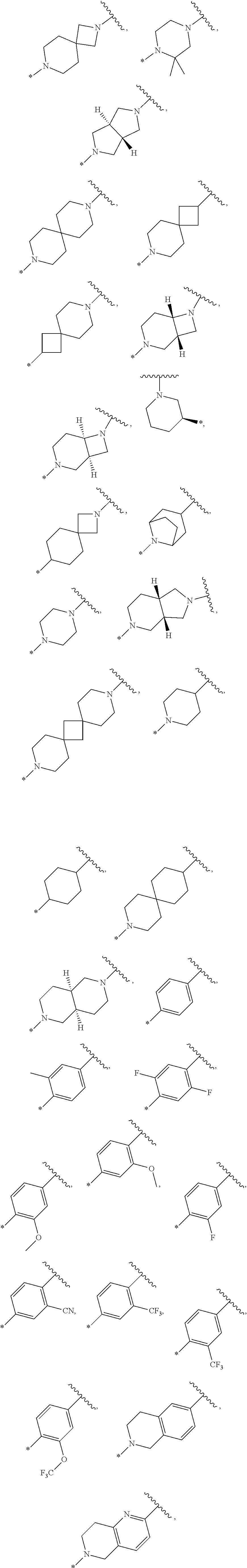

wherein [symbol] is a point of attachment to PLM and * is a point of attachment to L₂.

In some embodiments, ring A₂ is

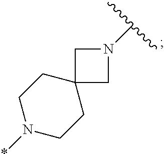

wherein [symbol] is a point of attachment to PLM and * is a point of attachment to L₂.

In some embodiments, ring A₂ is

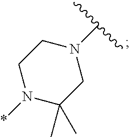

wherein [symbol] is a point of attachment to PLM and * is a point of attachment to L₂.

In some embodiments, ring A₂ is

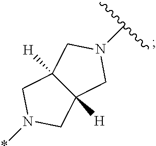

wherein [symbol] is a point of attachment to PLM and * is a point of attachment to L₂.

In some embodiments, ring A₂ is

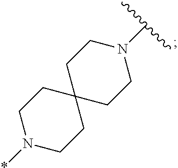

wherein [symbol] is a point of attachment to PLM and * is a point of attachment to L₂.

In some embodiments, ring A₂ is

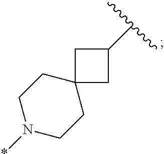

wherein [symbol] is a point of attachment to PLM and * is a point of attachment to L₂.

In some embodiments, ring A₂ is

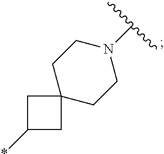

wherein [symbol] is a point of attachment to PLM and * is a point of attachment to L₂.

In some embodiments, ring A₂ is

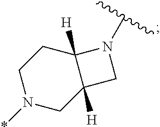

wherein [symbol] is a point of attachment to PLM and * is a point of attachment to L₂.

In some embodiments, ring A₂ is

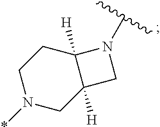

wherein [symbol] is a point of attachment to PLM and * is a point of attachment to L₂.

In some embodiments, ring A₂ is

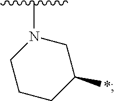

wherein [symbol] is a point of attachment to PLM and * is a point of attachment to L₂.

In some embodiments, ring A₂ is

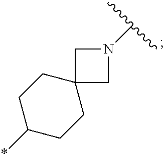

wherein [symbol] is a point of attachment to PLM and * is a point of attachment to L₂.

In some embodiments, ring A₂ is

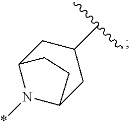

wherein [symbol] is a point of attachment to PLM and * is a point of attachment to L₂.

In some embodiments, ring A₂ is

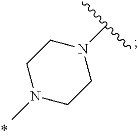

wherein [symbol] is a point of attachment to PLM and * is a point of attachment to L₂.

In other embodiments, ring A₂ is

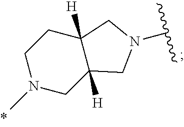

wherein [symbol] is a point of attachment to PLM and * is a point of attachment to L₂.

In other embodiments, ring A₂ is

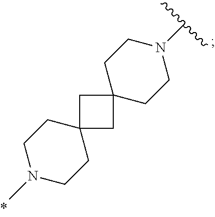

wherein [symbol] is a point of attachment to PLM and * is a point of attachment to L₂.

In other embodiments, ring A₂

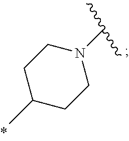

wherein [symbol] is a point of attachment to PLM and * is a point of attachment to L₂.

In other embodiments, ring A₂

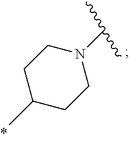

wherein [symbol] is a point of attachment to PLM and * is a point of attachment to L₂.

In other embodiments, ring A₂

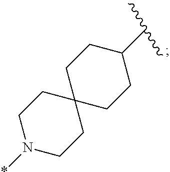

wherein [symbol] is a point of attachment to PLM and * is a point of attachment to L₂.

In other embodiments, ring A₂ is

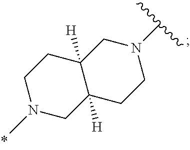

wherein [symbol] is a point of attachment to PLM and * is a point of attachment to L₂.

In other embodiments, ring A₂ is

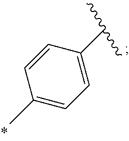

wherein [symbol] is a point of attachment to PLM and * is a point of attachment to L₂.

In other embodiments, ring A₂ is

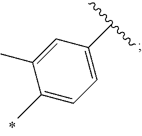

wherein [symbol] is a point of attachment to PLM and * is a point of attachment to L₂.

In other embodiments, ring A₂ is

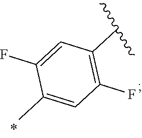

wherein [symbol] is a point of attachment to PLM and * is a point of attachment to L₂.

In other embodiments, ring A₂ is

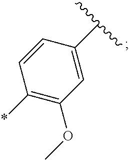

wherein [symbol] is a point of attachment to PLM and * is a point of attachment to L₂.

In other embodiments, ring A₂ is

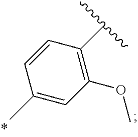

wherein [symbol] is a point of attachment to PLM and * is a point of attachment to L₂.

In other embodiments, ring A₂ is

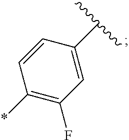

wherein [symbol] is a point of attachment to PLM and * is a point of attachment to L₂.

In other embodiments, ring A₂ is

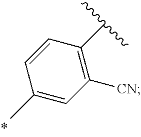

wherein [symbol] is a point of attachment to PLM and * is a point of attachment to L₂.

In other embodiments, ring A₂ is

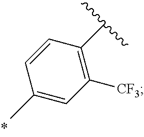

wherein [symbol] is a point of attachment to PLM and * is a point of attachment to L₂.

In yet other embodiments, ring A₂ is

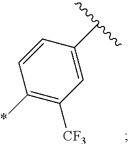

wherein [symbol] is a point of attachment to PLM and * is a point of attachment to L₂.

In yet other embodiments, ring A₂ is

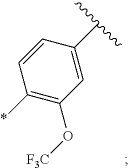

wherein [symbol] is a point of attachment to PLM and * is a point of attachment to L₂.

In yet other embodiments, ring A₂ is

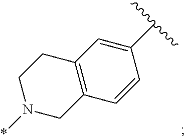

wherein [symbol] is a point of attachment to PLM and * is a point of attachment to L₂.

In yet other embodiments, ring A₂ is

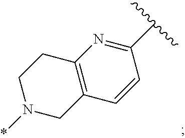

wherein [symbol] is a point of attachment to PLM and * is a point of attachment to L₂.

In yet other embodiments, ring A₂ is

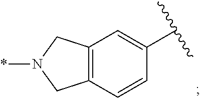

wherein [symbol] is a point of attachment to PLM and * is a point of attachment to L₂.

In yet other embodiments, ring A₂ is

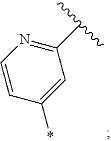

wherein [symbol] is a point of attachment to PLM and * is a point of attachment to L₂.

In yet other embodiments, ring A₂ is

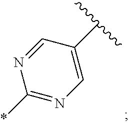

wherein [symbol] is a point of attachment to PLM and * is a point of attachment to L₂.

In yet other embodiments, ring A₂ is

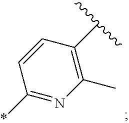

wherein [symbol] is a point of attachment to PLM and * is a point of attachment to L₂.

In yet other embodiments, ring A₂ is

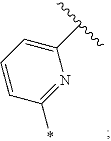

wherein [symbol] is a point of attachment to PLM and * is a point of attachment to L₂.

In yet other embodiments, ring A₂ is

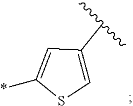

wherein [symbol] is a point of attachment to PLM and * is a point of attachment to L₂.

According to the disclosure, Y-PTM is a compound of formula IA-3

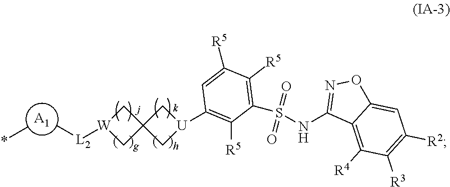

or a pharmaceutically acceptable salt thereof; wherein R², R³, R⁴, R⁵, R¹¹, L₂, ring A₁, W, U, j, k, g and h are as defined herein.

According to the disclosure, Y-PTM is a compound of formula IA-4

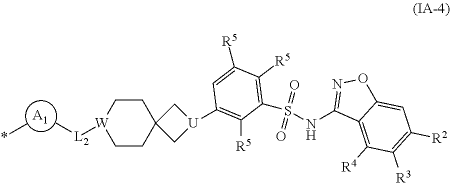

or a pharmaceutically acceptable salt thereof, wherein R², R³, R⁴, R⁵, R¹¹, L₂, ring A₁, W and U are as defined herein.

According to the disclosure, Y-PTM is a compound of formula IA-5

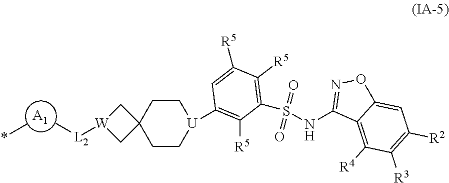

or a pharmaceutically acceptable salt thereof, wherein R², R³, R⁴, R⁵, R¹¹, L₂, ring A₁, W and U are as defined herein.

According to the disclosure, Y-PTM is a compound of formula IA-6

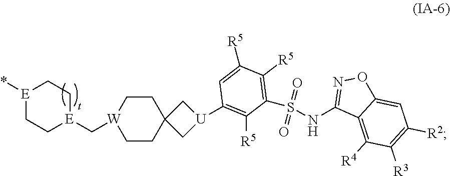

or a pharmaceutically acceptable salt thereof, wherein R², R³, R⁴, R⁵, R¹¹, W and U are as defined herein; and wherein
each E is independently CR_{II} or N; and
t is 0, 1, 2 or 3.

According to the disclosure, each E is independently CR₁₁ or N. In some embodiments, each E is N. In other embodiments, one E is CR₁₁ and the other E is N. In yet other embodiments, each E is CR₁₁.

According to the disclosure, t is 0, 1, 2 or 3. In some embodiments, t is 0. In some embodiments, t is 1. In other embodiments, t is 2. In other embodiments, t is 3.

According to the disclosure, R₁₁ is H, D, F, C₁₋₆alkyl, or C₁₋₆alkoxide. In some embodiments, R₁₁ is H. In other embodiments, R₁₁ is D. In other embodiments, R₁₁ is F. In other embodiments, R₁₁ is C₁₋₆alkyl. In other embodiments, R₁₁ is C₁₋₆alkoxide.

According to the disclosure, Y-PTM is a compound of formula IA-7

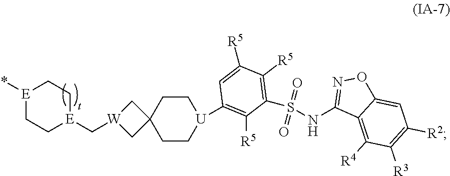

or a pharmaceutically acceptable salt thereof, wherein R², R³, R⁴, R⁵, R¹¹, W, U, E and t are as defined herein.

According to the disclosure, Y-PTM is a compound of formula IA-8

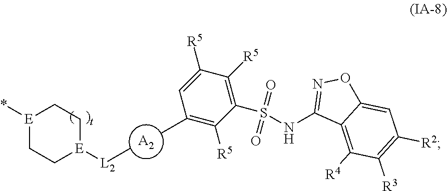

or a pharmaceutically acceptable salt thereof, wherein R², R³, R⁴, R⁵, R¹¹, L₂, ring A₂, E and t are as defined herein.

According to the disclosure, Y-PTM is a compound of formula IA-9

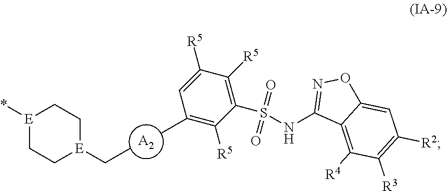

or a pharmaceutically acceptable salt thereof, wherein R², R³, R⁴, R⁵, R¹¹, ring A₂ and E are as defined herein.

According to the disclosure, any of the —C₁-C₆alkyl, C₁-C₈ alkoxide, —C₂-C₆alkenyl, —C₂-C₆alkynyl, aryl, heteroaryl, cycloalkyl, cycloalkenyl, heterocycloalkyl, heterocycloalkenyl, spirocycloalkyl, spiroheterocycloalkyl and spiroheterocycloalkenyl groups in any of R₁-R¹⁵ and ring Ais optionally substituted by 1-6 R^{f} groups.

In some embodiments, each R^{f} in Formula I is independently D, oxo, halogen, C₁-C₈ alkoxy, C₁-C₈ alkyl, C₁₋₆ haloalkyl, C₁₋₆ haloalkoxy, —OH, —CN, —NO₂, —C₂-C₆ alkenyl, —C₂-C₆ alkynyl, C₆₋₁₀ aryl, C₅₋₁₂ heteroaryl, C₃₋₈ cycloalkyl, C₃₋₈ cycloalkenyl, C₃₋₈ heterocycloalkyl, C₃₋₈ heterocycloalkenyl, —OR^{a}, —SR^{a}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)R^{c}R^{d}, —P(O)(OR^{b})(OR^{b}), —B(OR^{c})(OR^{d}), —S(O)₂R^{b}, —C(O)NR^{b}OR^{b}, —S(O)₂OR^{b}, —OS(O)₂OR^{b}, or —OPO(OR^{b})(OR^{b}); wherein said C₁-C₈ alkyl is optionally substituted by 1-6 groups selected from D, halogen, —OH, —CN, —OR^{a}, —SR^{a}, —NR^{a}R^{d}, or NR^{c}R^{d}.

In some embodiments, at least one R^{f} in Formula I is D. In some embodiments, at least one R^{f} in Formula I is oxo. In some embodiments, at least one R^{f} in Formula I is halogen. In some embodiments, at least one R^{f} in Formula I is C₁-C₈ alkoxy. In some embodiments, at least one R^{f} in Formula I is C₁-C₈ alkyl. In some embodiments, the C₁-C₈ alkyl is optionally substituted by 1-6 groups selected from D, halogen, —OH, —CN, —OR^{a}, —SR^{a}, —NR^{a}R^{d}, or NR^{c}R^{d}. In some embodiments, at least one R^{f} in Formula I is haloalkyl. In some embodiments, at least one R^{f} in Formula I is —OH. In some embodiments, R^{f} in Formula I is —CN. In some embodiments, at least one R^{f} in Formula I is —NO₂. In some embodiments, at least one R^{f} in Formula I is —C₂-C₆ alkenyl. In some embodiments, at least one R^{f} in Formula I is —C₂-C₆ alkynyl. In some embodiments, at least one R^{f} in Formula I is C₆-10 aryl. In some embodiments, at least one R^{f} in Formula I is C₅₋₁₂ heteroaryl. In some embodiments, at least one R^{f} in Formula I is C₃₋₈ cycloalkyl. In other embodiments, at least one R^{f} in Formula I is C₃₋₈ cycloalkenyl. In other embodiments, at least one R^{f} in Formula I is C₃₋₈ heterocycloalkyl. In other embodiments, at least one R^{f} in Formula I is C₃₋₈ heterocycloalkenyl. In other embodiments, at least one R^{f} in Formula I is —OR^{a}. In other embodiments, at least one R^{f} in Formula I is —SR^{a}. In other embodiments, at least one R^{f} in Formula I is —NR^{c}R^{d}. In other embodiments, at least one R^{f} in Formula I is —NR^{a}R^{c}. In other embodiments, at least one R^{f} in Formula I is —C(O)R^{b}. In other embodiments, at least one R^{f} in Formula I is —OC(O)R^{b}. In other embodiments, at least one R^{f} in Formula I is —C(O)OR^{b}. In other embodiments, at least one R^{f} in Formula I is —C(O)NR^{c}R^{d}. In yet other embodiments, at least one R^{f} in Formula I is —S(O)R^{b}. In yet other embodiments, at least one R^{f} in Formula I is —S(O)₂NR^{c}R^{d}. In yet other embodiments, at least one R^{f} in Formula I is —S(O)(═NR^{b})R^{b}. In yet other embodiments, at least one R^{f} in Formula I is —SF₅. In yet other embodiments, at least one R^{f} in Formula I is —P(O)R^{b}R^{b}. In yet other embodiments, at least one R^{f} in Formula I is —P(O)(OR^{b})(OR^{b}). In yet other embodiments, at least one R^{f} in Formula I is —B(OR^{c})(OR^{d}). In yet other embodiments, at least one R^{f} in Formula I is —S(O)₂R^{b}. In yet other embodiments, at least one R^{f} in Formula I is —C(O)NR^{b}OR^{b}. In yet other embodiments, at least one R^{f} in Formula I is —S(O)₂OR^{b}. In yet other embodiments, at least one R^{f} in Formula I is —OS(O)₂OR^{b}. In yet other embodiments, at least one R^{f} in Formula I is —OPO(OR^{b})(OR^{b}).

In some embodiments, each R^{a} in Formula I is independently H, D, —C(O)R^{b}, —C(O)OR^{c}, —C(O)NR^{c}R^{d}, —C(═NR^{b})NR^{b}R^{c}, —C(═NOR^{b})NR^{b}R^{c}, —C(═NCN)NR^{b}R^{c}, —P(OR^{c})₂, —P(O)R^{c}R^{b}, —P(O)OR^{c}OR^{b}, —S(O)R^{b}, —S(O)NR^{c}R^{d}, —S(O)₂R^{b}, —S(O)₂NR^{c}R^{d}, SiR^{b}₃, —C₁-C₁₀alkyl, —C₁-C₁₀ haloalkyl, —C₂-C₁₀ alkenyl, —C₂-C₁₀ alkynyl, C₆₋₁₀ aryl, C₃₋₈ cycloalkyl, C₃₋₈ cycloalkenyl, C₅₋₁₂ heteroaryl, C₃₋₈ heterocycloalkyl, or C₃₋₈ heterocycloalkenyl.

In some embodiments, at least one R^{a} in Formula I is H. In some embodiments, at least one R^{a} in Formula I is D. In some embodiments, at least one R^{a} in Formula I is —C(O)R^{b}. In some embodiments, at least one R^{a} in Formula I is —C(O)OR^{c}. In some embodiments, at least one R^{a} in Formula I is —C(O)NR^{c}R^{d}. In some embodiments, at least one R^{a} in Formula I is —C(═NR^{b})NR^{b}R^{c}. In some embodiments, at least one R^{a} in Formula I is C(═NOR^{b})NR^{b}R^{c}. In some embodiments, at least one R^{a} in Formula I is —C(═NCN)NR^{b}R^{c}.

In other embodiments, at least one R^{a} in Formula I is —P(OR^{c})₂, —P(O)R^{c}R^{b}, —P(O)OR^{c}OR^{b}, —S(O)R^{b}, —S(O)NR^{c}R^{d}, —S(O)₂R^{b}, —S(O)₂NR^{c}R^{d}, SiR^{b}₃, and the like. In yet other embodiments, at least one R^{a} in Formula I is —C₁-C₁₀alkyl, —C₁-C₁₀ haloalkyl, —C₂-C₁₀ alkenyl, —C₂-C₁₀ alkynyl, C₆-10 aryl, C₃₋₈ cycloalkyl, C₃₋₈ cycloalkenyl, C₅₋₁₂ heteroaryl, C₃₋₈ heterocycloalkyl, C₃₋₈ heterocycloalkenyl, and the like.

In some embodiments, each R^{b} in Formula I is independently H, D, —C₁-C₆ alkyl, —C₁-C₆ haloalkyl, —C₂-C₆ alkenyl, —C₂-C₆ alkynyl, C₆₋₁₀ aryl, C₃₋₈ cycloalkyl, C₃₋₈ cycloalkenyl, C₅₋₁₂ heteroaryl, C₃₋₈ heterocycloalkyl, or C₃₋₈ heterocycloalkenyl.

In some embodiments, at least one R^{b} in Formula I is H. In some embodiments, at least one R^{b} in Formula I is D. In some embodiments, at least one R^{b} in Formula I is —C₁-C₆ alkyl. In some embodiments, at least one R^{b} in Formula I is —C₁-C₆ haloalkyl. In some embodiments, at least one R^{b} in Formula I is —C₂-C₆ alkenyl. In some embodiments, at least one R^{b} in Formula I is —C₂-C₆ alkynyl. In other embodiments, at least one R^{b} in Formula I is C₆₋₁₀ aryl. In other embodiments, at least one R^{b} in Formula I is C₃₋₈ cycloalkyl. In other embodiments, at least one R^{b} in Formula I is C₃₋₈ cycloalkenyl. In other embodiments, at least one R^{b} in Formula I is C₅₋₁₂ heteroaryl. In other embodiments, at least one R^{b} in Formula I is C₃₋₈ heterocycloalkyl. In other embodiments, at least one R^{b} in Formula I is C₃₋₈ heterocycloalkenyl.

In some embodiments, each R^{c} or R^{d} in Formula I is independently H, D, —C₁-C₆ alkyl, —C₂-C₆ alkenyl, —C₂-C₆ alkynyl, C₆₋₁₀ aryl, C₃₋₈ cycloalkyl, C₃₋₈ cycloalkenyl, C₅₋₁₂ heteroaryl, C₃₋₈ heterocycloalkyl, or C₃₋₈ heterocycloalkenyl.

In some embodiments, R^{c} or R^{d} in Formula I is H. In some embodiments, R^{c} or R^{d} in Formula I is D. In some embodiments, R^{c} or R^{d} in Formula I is —C₁-C₁₀ alkyl. In some embodiments, R^{c} or R^{d} in Formula I is —C₂-C₆ alkenyl. In some embodiments, R^{c} or R^{d} in Formula I is —C₂-C₆ alkynyl. In other embodiments, R^{c} or R^{d} in Formula I is —OC₁-C₆alkyl. In other embodiments, R^{c} or R^{d} in Formula I is —O-cycloalkyl. In other embodiments, R^{c} or R^{d} in Formula I is C₆-10 aryl. In other embodiments, R^{c} or R^{d} in Formula I is C₃₋₈ cycloalkyl. In other embodiments, R^{c} or R^{d} in Formula I is C₃₋₈ cycloalkenyl. In other embodiments, R^{c} or R^{d} in Formula I is C₅₋₁₂ heteroaryl. In other embodiments, R^{c} or R^{d} in Formula I is C₃₋₈ heterocycloalkyl. In other embodiments, R^{c} or R^{d} in Formula I is C₃₋₈ heterocycloalkenyl. In yet other embodiments, R^{c} and R^{d} in Formula I, together with the atom to which they are both attached, form a monocyclic or multicyclic heterocycloalkyl, or a monocyclic or multicyclic heterocycloalkenyl group. In yet other embodiments, R^{c} and R^{d} in Formula I form a monocyclic heterocycloalkyl. In yet other embodiments, R^{c} and R^{d} in Formula I form a multicyclic heterocycloalkyl. In yet other embodiments, R^{c} and R^{d} in Formula I form a monocyclic heterocycloalkenyl group. In yet other embodiments, R^{c} and R^{d} in Formula I form a multicyclic heterocycloalkenyl group.

In some embodiments, the compounds disclosed herein are
N-(6-((1H-Pyrazol-1-yl)methyl)-4-methoxybenzo[d]isoxazol-3-yl)-3-(7-(((3S)-1-(1-(2,6-dioxopiperidin-3-yl)-3-methyl-1H-indazol-5-yl)pyrrolidin-3-yl)methyl)-2,7-diazaspiro[3.5]nonan-2-yl)benzenesulfonamide;
N-(6-((1H-Pyrazol-1-yl)methyl)-4-methoxybenzo[d]isoxazol-3-yl)-3-(7-((1-(1-(2,6-dioxopiperidin-3-yl)-3-methyl-2-oxo-2,3-dihydro-1H-benzo[d]imidazol-5-yl)piperidin-4-yl)methyl)-2,7-diazaspiro[3.5]nonan-2-yl)benzenesulfonamide;
N-(6-((1H-Pyrazol-1-yl)methyl)-4-methoxybenzo[d]isoxazol-3-yl)-3-(7-((1-(4-(2,6-dioxopiperidin-3-yl)phenyl)pyrrolidin-3-yl)methyl)-2,7-diazaspiro[3.5]nonan-2-yl)benzenesulfonamide;
N-(6-((1H-Pyrazol-1-yl)methyl)-4-methoxybenzo[d]isoxazol-3-yl)-3-(7-((1-(3-(2,6-dioxopiperidin-3-yl)-1-methyl-1H-pyrrolo[3,2-c]pyridin-6-yl)piperidin-4-yl)methyl)-2,7-diazaspiro[3.5]nonan-2-yl)benzenesulfonamide;
N-(6-((1H-Pyrazol-1-yl)methyl)-4-methoxybenzo[d]isoxazol-3-yl)-3-(7-((1-(2-(2,6-dioxopiperidin-3-yl)-1-oxo-1,2-dihydroisoquinolin-6-yl)piperidin-4-yl)methyl)-2,7-diazaspiro[3.5]nonan-2-yl)-2-methoxybenzenesulfonamide;
N-(6-((1H-Pyrazol-1-yl)methyl)-4-methoxybenzo[d]isoxazol-3-yl)-3-(7-((1-(1-(2,6-dioxopiperidin-3-yl)-3-methyl-1H-indazol-5-yl)piperidin-4-yl)methyl)-2,7-diazaspiro[3.5]nonan-2-yl)-2-methoxybenzenesulfonamide;
N-(6-((1H-Pyrazol-1-yl)methyl)-4-methoxybenzo[d]isoxazol-3-yl)-3-(7-((1-(3-(2,6-dioxopiperidin-3-yl)-1-methyl-1H-indazol-6-yl)piperidin-4-yl)methyl)-2,7-diazaspiro[3.5]nonan-2-yl)-2-methoxybenzenesulfonamide;
N-(6-((1H-Pyrazol-1-yl)methyl)-4-methoxybenzo[d]isoxazol-3-yl)-3-(7-((1-(6-(2,6-dioxopiperidin-3-yl)-1-methyl-1H-indazol-3-yl)piperidin-4-yl)methyl)-2,7-diazaspiro[3.5]nonan-2-yl)-2-methoxybenzenesulfonamide;
N-(6-((1H-Pyrazol-1-yl)methyl)-4-methoxybenzo[d]isoxazol-3-yl)-3-(7-((1-(3-(2,6-dioxopiperidin-3-yl)-1-methyl-1H-pyrrolo[2,3-b]pyridin-6-yl)piperidin-4-yl)methyl)-2,7-diazaspiro[3.5]nonan-2-yl)-2-methoxybenzenesulfonamide;
N-(6-((1H-Pyrazol-1-yl)methyl)-4-methoxybenzo[d]isoxazol-3-yl)-3-(7-((1-(3-(2,4-dioxotetrahydropyrimidin-1(2H)-yl)-1-methyl-1H-indazol-6-yl)piperidin-4-yl)methyl)-2,7-diazaspiro[3.5]nonan-2-yl)-2,4-dimethoxybenzenesulfonamide;
N-(6-((1H-Pyrazol-1-yl)methyl)-4-methoxybenzo[d]isoxazol-3-yl)-3-(7-((1-(3-(2,6-dioxopiperidin-3-yl)-1-methyl-1H-indazol-6-yl)piperidin-4-yl)methyl)-2,7-diazaspiro[3.5]nonan-2-yl)-2,6-dimethoxybenzenesulfonamide;
N-(6-((1H-Pyrazol-1-yl)methyl)-4-methoxybenzo[d]isoxazol-3-yl)-4-(7-((1-(1-(2,6-dioxopiperidin-3-yl)-3-methyl-1H-indazol-5-yl)piperidin-4-yl)methyl)-2,7-diazaspiro[3.5]nonan-2-yl)benzenesulfonamide;
N-(6-((1H-Pyrazol-1-yl)methyl)-4-methoxybenzo[d]isoxazol-3-yl)-4-(7-((1-(2-(2,6-dioxopiperidin-3-yl)-1-oxo-1,2-dihydroisoquinolin-6-yl)piperidin-4-yl)methyl)-2,7-diazaspiro[3.5]nonan-2-yl)benzenesulfonamide;
N-(6-((1H-Pyrazol-1-yl)methyl)-4-methoxybenzo[d]isoxazol-3-yl)-4-(7-((1-(1-(2,6-dioxopiperidin-3-yl)-3-methyl-1H-indazol-5-yl)piperidin-4-yl)methyl)-2,7-diazaspiro[3.5]nonan-2-yl)-2-methoxybenzenesulfonamide;
N-(6-((1H-Pyrazol-1-yl)methyl)-4-methoxybenzo[d]isoxazol-3-yl)-4-(7-((1-(1-(2,6-dioxopiperidin-3-yl)-3-methyl-2-oxo-2,3-dihydro-1H-benzo[d]imidazol-5-yl)piperidin-4-yl)methyl)-2,7-diazaspiro[3.5]nonan-2-yl)-2-methoxybenzenesulfonamide;
N-(6-((1H-Pyrazol-1-yl)methyl)-4-methoxybenzo[d]isoxazol-3-yl)-4-(4-((1-(1-(2,6-dioxopiperidin-3-yl)-3-methyl-2-oxo-2,3-dihydro-1H-benzo[d]imidazol-5-yl)piperidin-4-yl)methyl)-3,3-dimethylpiperazin-1-yl)benzenesulfonamide;
N-(6-((1H-Pyrazol-1-yl)methyl)-4-methoxybenzo[d]isoxazol-3-yl)-4-((3aS,6aS)-5-((1-(1-(2,6-dioxopiperidin-3-yl)-3-methyl-2-oxo-2,3-dihydro-1H-benzo[d]imidazol-5-yl)piperidin-4-yl)methyl)hexahydropyrrolo[3,4-c]pyrrol-2(1H)-yl)benzenesulfonamide;
N-(6-((1H-Pyrazol-1-yl)methyl)-4-methoxybenzo[d]isoxazol-3-yl)-4-((3aR,6aS)-5-((1-(1-(2,6-dioxopiperidin-3-yl)-3-methyl-2-oxo-2,3-dihydro-1H-benzo[d]imidazol-5-yl)piperidin-4-yl)methyl)hexahydropyrrolo[3,4-c]pyrrol-2(1H)-yl)-2-methoxybenzenesulfonamide;
N-(6-((1H-Pyrazol-1-yl)methyl)-4-methoxybenzo[d]isoxazol-3-yl)-3-(9-((1-(4-(2,6-dioxopiperidin-3-yl)phenyl)piperidin-4-yl)methyl)-3,9-diazaspiro[5.5]undecan-3-yl)benzenesulfonamide;
N-(6-((1H-Pyrazol-1-yl)methyl)-4-methoxybenzo[d]isoxazol-3-yl)-3-(9-((1-(3-(2,6-dioxopiperidin-3-yl)-1-methyl-1H-indazol-6-yl)piperidin-4-yl)methyl)-3,9-diazaspiro[5.5]undecan-3-yl)benzenesulfonamide;
N-(6-((1H-Pyrazol-1-yl)methyl)-4-methoxybenzo[d]isoxazol-3-yl)-3-(7-((1-(3-(2,6-dioxopiperidin-3-yl)-1-methyl-1H-indazol-6-yl)piperidin-4-yl)methyl)-2,7-diazaspiro[3.5]nonan-2-yl)-2-(2,2,2-trifluoroethoxy)benzenesulfonamide;
N-(6-((1H-Pyrazol-1-yl)methyl)-4-methoxybenzo[d]isoxazol-3-yl)-2-chloro-3-(7-((1-(3-(2,6-dioxopiperidin-3-yl)-1-methyl-1H-indazol-6-yl)piperidin-4-yl)methyl)-2,7-diazaspiro[3.5]nonan-2-yl)benzenesulfonamide;
N-(6-((1H-Pyrazol-1-yl)methyl)-4-methoxybenzo[d]isoxazol-3-yl)-3-(7-((1-(3-(2,6-dioxopiperidin-3-yl)-1-methyl-1H-indazol-6-yl)piperidin-4-yl)methyl)-2,7-diazaspiro[3.5]nonan-2-yl)-5-fluoro-2-methoxybenzenesulfonamide;
N-(6-((1H-Pyrazol-1-yl)methyl)-4-methoxybenzo[d]isoxazol-3-yl)-5-chloro-3-(7-((1-(3-(2,6-dioxopiperidin-3-yl)-1-methyl-1H-indazol-6-yl)piperidin-4-yl)methyl)-2,7-diazaspiro[3.5]nonan-2-yl)-2-methoxybenzenesulfonamide;
N-(6-((1H-Pyrazol-1-yl)methyl)-4-methoxybenzo[d]isoxazol-3-yl)-3-(7-((1-(3-(2,6-dioxopiperidin-3-yl)-1-methyl-1H-indazol-6-yl)piperidin-4-yl)methyl)-2,7-diazaspiro[3.5]nonan-2-yl)-2-methoxy-5-methylbenzenesulfonamide;
N-(6-((1H-pyrazol-1-yl)methyl)-4-methoxybenzo[d]isoxazol-3-yl)-3-(7-((1-(3-(2,6-dioxopiperidin-3-yl)-1-methyl-1H-indazol-6-yl)piperidin-4-yl)methyl)-2,7-diazaspiro[3.5]nonan-2-yl)-2-(trifluoromethyl)benzenesulfonamide;
N-(6-((1H-pyrazol-1-yl)methyl)-4-methoxybenzo[d]isoxazol-3-yl)-3-(7-((1-(3-(2,6-dioxopiperidin-3-yl)-1-methyl-1H-indazol-6-yl)piperidin-4-yl)methyl)-2,7-diazaspiro[3.5]nonan-2-yl)-2-fluorobenzenesulfonamide;
N-(6-((1H-Pyrazol-1-yl)methyl)-4-methoxybenzo[d]isoxazol-3-yl)-3-(7-((1-(3-(2,6-dioxopiperidin-3-yl)-1-methyl-1H-indazol-6-yl)piperidin-4-yl)methyl)-2,7-diazaspiro[3.5]nonan-2-yl)-5-fluoro-2,6-dimethoxybenzenesulfonamide;
N-(6-((1H-Pyrazol-1-yl)methyl)-4-methoxybenzo[d]isoxazol-3-yl)-5-(7-((1-(4-(2,6-dioxopiperidin-3-yl)phenyl)piperidin-4-yl)methyl)-2,7-diazaspiro[3.5]nonan-2-yl)-2-methylbenzenesulfonamide;
N-(6-((1H-Pyrazol-1-yl)methyl)-4-methoxybenzo[d]isoxazol-3-yl)-3-(7-((1-(4-(2,6-dioxopiperidin-3-yl)phenyl)piperidin-4-yl)methyl)-2,7-diazaspiro[3.5]nonan-2-yl)-2-methylbenzenesulfonamide;
N-(6-((1H-Pyrazol-1-yl)methyl)-4-methoxybenzo[d]isoxazol-3-yl)-4-(7-((1-(3-(2,6-dioxopiperidin-3-yl)-1-methyl-1H-indazol-6-yl)piperidin-4-yl)methyl)-2,7-diazaspiro[3.5]nonan-2-yl)-2-methylbenzenesulfonamide;
N-(6-((1H-Pyrazol-1-yl)methyl)-4-methoxybenzo[d]isoxazol-3-yl)-4-(7-((1-(3-(2,6-dioxopiperidin-3-yl)-1-methyl-1H-indazol-6-yl)piperidin-4-yl)methyl)-2,7-diazaspiro[3.5]nonan-2-yl)-2-ethylbenzenesulfonamide;
N-(6-((1H-Pyrazol-1-yl)methyl)-4-methoxybenzo[d]isoxazol-3-yl)-4-(7-((1-(3-(2,6-dioxopiperidin-3-yl)-1-methyl-1H-indazol-6-yl)piperidin-4-yl)methyl)-2,7-diazaspiro[3.5]nonan-2-yl)-2-(trifluoromethoxy)benzenesulfonamide;
N-(6-((1H-Pyrazol-1-yl)methyl)-4-methoxybenzo[d]isoxazol-3-yl)-3-(7-((1-(3-(2,6-dioxopiperidin-3-yl)-1-methyl-1H-indazol-6-yl)piperidin-4-yl)methyl)-7-azaspiro[3.5]nonan-2-yl)benzenesulfonamide;
N-(6-((1H-Pyrazol-1-yl)methyl)-4-methoxybenzo[d]isoxazol-3-yl)-3-(2-((4-(3-(2,4-dioxotetrahydropyrimidin-1(2H)-yl)-1-methyl-1H-indazol-6-yl)piperidin-1-yl)methyl)-7-azaspiro[3.5]nonan-7-yl)benzenesulfonamide;
N-(6-((1H-Pyrazol-1-yl)methyl)-4-methoxybenzo[d]isoxazol-3-yl)-3-(2-((4-(3-(2,6-dioxopiperidin-3-yl)-1-methyl-1H-indazol-6-yl)piperidin-1-yl)methyl)-7-azaspiro[3.5]nonan-7-yl)benzenesulfonamide;
N-(6-((1H-Pyrazol-1-yl)methyl)-4-methoxybenzo[d]isoxazol-3-yl)-3-(2-((4-((1-(2,6-dioxopiperidin-3-yl)-3-methyl-2-oxo-2,3-dihydro-1H-benzo[d]imidazol-4-yl)ethynyl)piperidin-1-yl)methyl)-7-azaspiro[3.5]nonan-7-yl)benzenesulfonamide;
N-(6-((1H-Pyrazol-1-yl)methyl)-4-methoxybenzo[d]isoxazol-3-yl)-4′-((4-(3-(2,4-dioxotetrahydropyrimidin-1(2H)-yl)-1-methyl-1H-indazol-6-yl)piperidin-1-yl)methyl)-2,4-dimethoxy-[1,1′-biphenyl]-3-sulfonamide;
or a pharmaceutically acceptable salt thereof.

In some embodiments, the compounds disclosed herein are
3-(7-((1-(3-(2,6-Dioxopiperidin-3-yl)-1-methyl-1H-indazol-6-yl)piperidin-4-yl)methyl)-2,7-diazaspiro[3.5]nonan-2-yl)-5-fluoro-2,6-dimethoxy-N-(4-methoxy-6-(methoxymethyl)-benzo[d]isoxazol-3-yl)benzenesulfonamide;
3-(7-((1-(3-(2,4-Dioxotetrahydropyrimidin-1(2H)-yl)-1-methyl-1H-indazol-6-yl)-piperidin-4-yl)methyl)-2,7-diazaspiro[3.5]nonan-2-yl)-5-fluoro-2,6-dimethoxy-N-(4-methoxy-6-(methoxymethyl)benzo[d]isoxazol-3-yl)benzenesulfonamide;
3-(7-((1-(3-(2,6-Dioxopiperidin-3-yl)-1-methyl-1H-pyrrolo[2,3-b]pyridin-6-yl)piperidin-4-yl)methyl)-2,7-diazaspiro[3.5]nonan-2-yl)-5-fluoro-2,6-dimethoxy-N-(4-methoxy-6-(methoxy-methyl)benzo[d]isoxazol-3-yl)benzenesulfonamide;
3-(2-((1-(3-(2,6-Dioxopiperidin-3-yl)-1-methyl-1H-indazol-6-yl)piperidin-4-yl)methyl)-1,2,3,4-tetrahydroisoquinolin-6-yl)-5-fluoro-2,6-dimethoxy-N-(4-methoxy-6-(methoxymethyl)-benzo[d]isoxazol-3-yl)benzenesulfonamide;
3-(2-((1-(3-(2,4-Dioxotetrahydropyrimidin-1(2H)-yl)-1-methyl-1H-indazol-6-yl)-piperidin-4-yl)methyl)-1,2,3,4-tetrahydroisoquinolin-6-yl)-5-fluoro-2,6-dimethoxy-N-(4-methoxy-6-(methoxymethyl)benzo[d]isoxazol-3-yl)benzenesulfonamide;
4′-((4-(3-(2,6-Dioxopiperidin-3-yl)-1-methyl-1H-indazol-6-yl)piperidin-1-yl)methyl)-5-fluoro-2,4-dimethoxy-N-(4-methoxy-6-(methoxymethyl)benzo[d]isoxazol-3-yl)-3′-methyl-[1,1′-biphenyl]-3-sulfonamide;
4′-((4-(3-(2,4-Dioxotetrahydropyrimidin-1(2H)-yl)-1-methyl-1H-indazol-6-yl)piperidin-1-yl)methyl)-5-fluoro-2,4-dimethoxy-N-(4-methoxy-6-(methoxymethyl)benzo-[d]isoxazol-3-yl)-3′-methyl-[1,1′-biphenyl]-3-sulfonamide;
N-(6-((1H-Pyrazol-1-yl)methyl)-4-methoxybenzo[d]isoxazol-3-yl)-3-(7-((1-(3-(2,4-dioxotetrahydropyrimidin-1(2H)-yl)-1-methyl-1H-indazol-6-yl)piperidin-4-yl)methyl)-2,7-diazaspiro[3.5]nonan-2-yl)-5-fluoro-2,6-dimethoxybenzenesulfonamide;
rac-3-((cis)-3-((1-(3-(2,4-Dioxotetrahydropyrimidin-1(2H)-yl)-1-methyl-1H-indazol-6-yl)piperidin-4-yl)methyl)-3,7-diazabicyclo[4.2.0]octan-7-yl)-5-fluoro-2,6-dimethoxy-N-(4-methoxy-6-(methoxymethyl)benzo[d]isoxazol-3-yl)benzenesulfonamide;
3-((1-(3-(2,6-Dioxopiperidin-3-yl)-1-methyl-1H-pyrrolo[2,3-b]pyridin-6-yl)piperidin-4-yl)methyl)-3,7-diazabicyclo[4.2.0]octan-7-yl)-5-fluoro-2,6-dimethoxy-N-(4-methoxy-6-(methoxymethyl)benzo[d]isoxazol-3-yl)benzenesulfonamide;
rac-3-((cis)-3-((1-(3-(2,4-Dioxotetrahydropyrimidin-1(2H)-yl)-1-methyl-1H-indazol-6-yl)piperidin-4-yl)methyl)-3,7-diazabicyclo[4.2.0]octan-7-yl)-2-methoxy-N-(4-methoxy-6-(methoxymethyl)benzo[d]isoxazol-3-yl)-5-(trifluoromethyl)benzenesulfonamide;
3-((1-(3-(2,6-Dioxopiperidin-3-yl)-1-methyl-1H-pyrrolo[2,3-b]pyridin-6-yl)piperidin-4-yl)methyl)-3,7-diazabicyclo[4.2.0]octan-7-yl)-2-methoxy-N-(4-methoxy-6-(methoxymethyl)-benzo[d]isoxazol-3-yl)-5-(trifluoromethyl)benzenesulfonamide;
N-(6-((1H-pyrazol-1-yl)methyl)-4-methoxybenzo[d]isoxazol-3-yl)-3-(6-((1-(3-(2,4-dioxotetrahydropyrimidin-1(2H)-yl)-1-methyl-1H-indazol-6-yl)piperidin-4-yl)methyl)-5,6,7,8-tetrahydro-1,6-naphthyridin-2-yl)-5-fluoro-2,6-dimethoxybenzene-sulfonamide;
N-(6-((1H-pyrazol-1-yl)methyl)-4-methoxybenzo[d]isoxazol-3-yl)-3-(7-((1-(4-(2,6-dioxopiperidin-3-yl)phenyl)piperidin-4-yl)methyl)-2,7-diazaspiro[3.5]nonan-2-yl)-5-fluoro-2,6-dimethoxybenzenesulfonamide;
rac-3-(cis)-N-(6-((1H-pyrazol-1-yl)methyl)-4-methoxybenzo[d]isoxazol-3-yl)-3-((1-(3-(2,4-dioxotetrahydropyrimidin-1(2H)-yl)-1-methyl-1H-indazol-6-yl)-piperidin-4-yl)methyl)-3,7-diazabicyclo[4.2.0]octan-7-yl)-5-fluoro-2,6-dimethoxy-benzenesulfonamide;
or a pharmaceutically acceptable salt thereof.

In some embodiments, the compounds disclosed herein are
N-(6-((1H-Pyrazol-1-yl)methyl)-4-methoxybenzo[d]isoxazol-3-yl)-3-(7-((4-(3-(2,4-dioxotetrahydropyrimidin-1(2H)-yl)-1-methyl-1H-indazol-6-yl)piperazin-1-yl)methyl)-2-azaspiro[3.5]nonan-2-yl)benzenesulfonamide;
N-(6-((1H-Pyrazol-1-yl)methyl)-4-methoxybenzo[d]isoxazol-3-yl)-3-(7-((4-(4-(2,6-dioxopiperidin-3-yl)phenyl)piperidin-1-yl)methyl)-2-azaspiro[3.5]nonan-2-yl)-5-fluoro-2,6-dimethoxybenzenesulfonamide;
N-(6-((1H-Pyrazol-1-yl)methyl)-4-methoxybenzo[d]isoxazol-3-yl)-3-(7-((1-(3-(2,6-dioxopiperidin-3-yl)-1-methyl-1H-indazol-6-yl)piperidin-4-yl)methyl)-2,7-diazaspiro[3.5]nonan-2-yl)benzenesulfonamide;
N-(6-((1H-Pyrazol-1-yl)methyl)-4-methoxybenzo[d]isoxazol-3-yl)-3-(7-((1-(3-(2,4-dioxotetrahydropyrimidin-1(2H)-yl)-1-methyl-1H-pyrazolo[3,4-b]pyridin-6-yl)piperidin-4-yl)methyl)-2,7-diazaspiro[3.5]nonan-2-yl)-2,6-dimethoxybenzenesulfonamide;
N-(6-((1H-Pyrazol-1-yl)methyl)-4-methoxybenzo[d]isoxazol-3-yl)-3-(7-((1-(3-(2,6-dioxopiperidin-3-yl)-1-methyl-1H-pyrazolo[3,4-b]pyridin-6-yl)piperidin-4-yl)methyl)-2,7-diazaspiro[3.5]nonan-2-yl)-5-fluoro-2,6-dimethoxybenzenesulfonamide;
N-(6-((1H-Pyrazol-1-yl)methyl)-4-methoxybenzo[d]isoxazol-3-yl)-3-(7-((1-(3-(2,4-dioxotetrahydropyrimidin-1(2H)-yl)-7-fluoro-1-methyl-1H-indazol-6-yl)piperidin-4-yl)methyl)-2,7-diazaspiro[3.5]nonan-2-yl)-5-fluoro-2,6-dimethoxybenzenesulfonamide;
N-(6-((1H-Pyrazol-1-yl)methyl)-4-methoxybenzo[d]isoxazol-3-yl)-3-(4-((7-(3-(2,4-dioxotetrahydropyrimidin-1(2H)-yl)-1-methyl-1H-indazol-6-yl)-7-azaspiro[3.5]nonan-2-yl)methyl)piperazin-1-yl)-5-fluoro-2,6-dimethoxybenzenesulfonamide;
N-(6-((1H-Pyrazol-1-yl)methyl)-4-methoxybenzo[d]isoxazol-3-yl)-3-(6-((1-(3-(2,6-dioxopiperidin-3-yl)-1-methyl-1H-indazol-6-yl)piperidin-4-yl)methyl)-5,6,7,8-tetrahydro-1,6-naphthyridin-2-yl)-5-fluoro-2,6-dimethoxybenzenesulfonamide;
N-(6-((1H-Pyrazol-1-yl)methyl)-4-methoxybenzo[d]isoxazol-3-yl)-4′-((4-(3-(2,4-dioxotetrahydropyrimidin-1(2H)-yl)-1-methyl-1H-indazol-6-yl)piperidin-1-yl)methyl)-2,4-dimethoxy-3′-methyl-[1,1′-biphenyl]-3-sulfonamide;
N-(6-((1H-Pyrazol-1-yl)methyl)-4-methoxybenzo[d]isoxazol-3-yl)-4′-((4-(3-(2,4-dioxotetrahydropyrimidin-1(2H)-yl)-1-methyl-1H-indazol-6-yl)piperidin-1-yl)methyl)-2,2′,4-trimethoxy-[1,1′-biphenyl]-3-sulfonamide;
N-(6-((1H-Pyrazol-1-yl)methyl)-4-methoxybenzo[d]isoxazol-3-yl)-4′-((4-(3-(2,6-dioxopiperidin-3-yl)-1-methyl-1H-pyrrolo[2,3-b]pyridin-6-yl)piperidin-1-yl)methyl)-2,3′,4-trimethoxy-[1,1′-biphenyl]-3-sulfonamide;
N-(6-((1H-Pyrazol-1-yl)methyl)-4-methoxybenzo[d]isoxazol-3-yl)-4′-((4-(3-(2,6-dioxopiperidin-3-yl)-1-methyl-1H-indazol-6-yl)piperidin-1-yl)methyl)-2,4-dimethoxy-3′-(trifluoromethoxy)-[1,1′-biphenyl]-3-sulfonamide;
N-(6-((1H-Pyrazol-1-yl)methyl)-4-methoxybenzo[d]isoxazol-3-yl)-4′-((4-(3-(2,4-dioxotetrahydropyrimidin-1(2H)-yl)-1-methyl-1H-indazol-6-yl)piperidin-1-yl)methyl)-2′-fluoro-2,4-dimethoxy-[1,1′-biphenyl]-3-sulfonamide;
N-(6-((1H-Pyrazol-1-yl)methyl)-4-methoxybenzo[d]isoxazol-3-yl)-4′-((4-(3-(2,6-dioxopiperidin-3-yl)-1-methyl-1H-indazol-6-yl)piperidin-1-yl)methyl)-3′-fluoro-2,4-dimethoxy-[1,1′-biphenyl]-3-sulfonamide;
N-(6-((1H-Pyrazol-1-yl)methyl)-4-methoxybenzo[d]isoxazol-3-yl)-4′-((4-(3-(2,6-dioxopiperidin-3-yl)-1-methyl-1H-indazol-6-yl)piperidin-1-yl)methyl)-2′,5′-difluoro-2,4-dimethoxy-[1,1′-biphenyl]-3-sulfonamide;
N-(6-((1H-Pyrazol-1-yl)methyl)-4-methoxybenzo[d]isoxazol-3-yl)-4′-((4-(3-(2,4-dioxotetrahydropyrimidin-1(2H)-yl)-1-methyl-1H-indazol-6-yl)piperidin-1-yl)methyl)-2,4-dimethoxy-3′-(trifluoromethyl)-[1,1′-biphenyl]-3-sulfonamide;
N-(6-((1H-Pyrazol-1-yl)methyl)-4-methoxybenzo[d]isoxazol-3-yl)-4′-((4-(3-(2,6-dioxopiperidin-3-yl)-1-methyl-1H-indazol-6-yl)piperidin-1-yl)methyl)-2,4-dimethoxy-2′-(trifluoromethyl)-[1,1′-biphenyl]-3-sulfonamide;
N-(6-((1H-Pyrazol-1-yl)methyl)-4-methoxybenzo[d]isoxazol-3-yl)-2′-cyano-4′-((4-(3-(2,4-dioxotetrahydropyrimidin-1(2H)-yl)-1-methyl-1H-indazol-6-yl)piperidin-1-yl)methyl)-2,4-dimethoxy-[1,1′-biphenyl]-3-sulfonamide;
N-(6-((1H-Pyrazol-1-yl)methyl)-4-methoxybenzo[d]isoxazol-3-yl)-3-(5-((4-(3-(2,6-dioxopiperidin-3-yl)-1-methyl-1H-indazol-6-yl)piperidin-1-yl)methyl)thiophen-3-yl)-2,6-dimethoxybenzenesulfonamide;
N-(6-((1H-Pyrazol-1-yl)methyl)-4-methoxybenzo[d]isoxazol-3-yl)-3-(2-((1-(3-(2,6-dioxopiperidin-3-yl)-1-methyl-1H-indazol-6-yl)piperidin-4-yl)methyl)-1,2,3,4-tetrahydro-isoquinolin-6-yl)-5-fluoro-2,6-dimethoxybenzenesulfonamide;
N-(6-((1H-Pyrazol-1-yl)methyl)-4-methoxybenzo[d]isoxazol-3-yl)-3-(2-((1-(3-(2,4-dioxotetrahydropyrimidin-1(2H)-yl)-1-methyl-1H-indazol-6-yl)piperidin-4-yl)methyl)-isoindolin-5-yl)-5-fluoro-2,6-dimethoxybenzenesulfonamide;
N-(6-((1H-Pyrazol-1-yl)methyl)-4-methoxybenzo[d]isoxazol-3-yl)-3-(2-((1-(3-(2,4-dioxotetrahydropyrimidin-1(2H)-yl)-1-methyl-1H-indazol-6-yl)piperidin-4-yl)methyl)-1,2,3,4-tetrahydroisoquinolin-6-yl)-6-fluoro-2-methoxybenzenesulfonamide;
N-(6-((1H-Pyrazol-1-yl)methyl)-4-methoxybenzo[d]isoxazol-3-yl)-3-(2-((1-(3-(2,4-dioxotetrahydropyrimidin-1(2H)-yl)-1-methyl-1H-indazol-6-yl)piperidin-4-yl)methyl)-1,2,3,4-tetrahydroisoquinolin-7-yl)-2-methoxy-5-(trifluoromethyl)benzenesulfonamide;
N-(6-((1H-Pyrazol-1-yl)methyl)-4-methoxybenzo[d]isoxazol-3-yl)-5-(tert-butyl)-3-(6-((1-(3-(2,4-dioxotetrahydropyrimidin-1(2H)-yl)-1-methyl-1H-indazol-6-yl)piperidin-4-yl)methyl)-5,6,7,8-tetrahydro-1,6-naphthyridin-2-yl)-2-methoxybenzenesulfonamide;
N-(6-((1H-Pyrazol-1-yl)methyl)-4-methoxybenzo[d]isoxazol-3-yl)-3-(2-((4-(3-(2,6-dioxopiperidin-3-yl)-1-methyl-1H-indazol-6-yl)piperidin-1-yl)methyl)pyrimidin-5-yl)-2,6-dimethoxybenzenesulfonamide;
N-(6-((1H-Pyrazol-1-yl)methyl)-4-methoxybenzo[d]isoxazol-3-yl)-3-(6-((4-(3-(2,6-dioxopiperidin-3-yl)-1-methyl-1H-indazol-6-yl)piperidin-1-yl)methyl)-2-methylpyridin-3-yl)-5-fluoro-2,6-dimethoxybenzenesulfonamide;
N-(6-((1H-Pyrazol-1-yl)methyl)-4-methoxybenzo[d]isoxazol-3-yl)-4′-(4-(3-(2,6-dioxopiperidin-3-yl)-1-methyl-1H-indazol-6-yl)piperidine-1-carbonyl)-2,4-dimethoxy-[1,1′-biphenyl]-3-sulfonamide;
N-(6-((1H-Pyrazol-1-yl)methyl)-4-methoxybenzo[d]isoxazol-3-yl)-3-(1-((1-(3-(2,4-dioxotetrahydropyrimidin-1(2H)-yl)-1-methyl-1H-indazol-6-yl)piperidin-4-yl)methyl)-piperidin-4-yl)-5-fluoro-2,6-dimethoxybenzenesulfonamide;
N-(6-((1H-Pyrazol-1-yl)methyl)-4-methoxybenzo[d]isoxazol-3-yl)-3-(4-((4-(3-(2,4-dioxotetrahydropyrimidin-1(2H)-yl)-1-methyl-1H-indazol-6-yl)piperidin-1-yl)methyl)cyclo-hexyl)-5-fluoro-2,6-dimethoxybenzenesulfonamide;
N-(6-((1H-Pyrazol-1-yl)methyl)-4-methoxybenzo[d]isoxazol-3-yl)-3-(3-((1-(3-(2,6-dioxopiperidin-3-yl)-1-methyl-1H-indazol-6-yl)piperidin-4-yl)methyl)-3-azaspiro[5.5]-undecan-9-yl)-2,6-dimethoxybenzenesulfonamide;
N-(6-((1H-Pyrazol-1-yl)methyl)-4-methoxybenzo[d]isoxazol-3-yl)-3-((1-((1-(3-(2,6-dioxopiperidin-3-yl)-1-methyl-1H-pyrrolo[2,3-b]pyridin-6-yl)piperidin-4-yl)methyl)piperidin-4-yl)methyl)-5-fluoro-2,6-dimethoxybenzenesulfonamide;
N-(6-((1H-Pyrazol-1-yl)methyl)-4-methoxybenzo[d]isoxazol-3-yl)-4′-((4-(3-(2,4-dioxotetrahydropyrimidin-1(2H)-yl)-1-methyl-1H-indazol-6-yl)piperidin-1-yl)methyl)-2,4-dimethoxy-[1,1′-biphenyl]-3-sulfonamide;
N-(6-((1H-Pyrazol-1-yl)methyl)-4-methoxybenzo[d]isoxazol-3-yl)-4′-((4-(3-(2,4-dioxotetrahydropyrimidin-1(2H)-yl)-1-methyl-1H-indazol-6-yl)piperazin-1-yl)methyl)-5-fluoro-2,4-dimethoxy-2′-(trifluoromethyl)-[1,1′-biphenyl]-3-sulfonamide;
N-(6-((1H-Pyrazol-1-yl)methyl)-4-methoxybenzo[d]isoxazol-3-yl)-4′-((3-(3-(2,6-dioxopiperidin-3-yl)-1-methyl-1H-indazol-6-yl)pyrrolidin-1-yl)methyl)-5-fluoro-2,4-dimethoxy-2′-(trifluoromethyl)-[1,1′-biphenyl]-3-sulfonamide;
rac-N-(6-((1H-Pyrazol-1-yl)methyl)-4-methoxybenzo[d]isoxazol-3-yl)-3-((3a,7a)-5-((1-(3-(2,4-dioxotetrahydropyrimidin-1(2H)-yl)-1-methyl-1H-indazol-6-yl)piperidin-4-yl)methyl)octahydro-2H-pyrrolo[3,4-c]pyridin-2-yl)-5-fluoro-2,6-dimethoxy-benzenesulfonamide;
N-(6-((1H-Pyrazol-1-yl)methyl)-4-methoxybenzo[d]isoxazol-3-yl)-3-((4aR,8aR)-6-((1-(3-(2,4-dioxotetrahydropyrimidin-1(2H)-yl)-1-methyl-1H-indazol-6-yl)piperidin-4-yl)methyl)octahydro-2,6-naphthyridin-2(1H)-yl)-5-fluoro-2,6-dimethoxybenzenesulfonamide;
N-(6-((1H-Pyrazol-1-yl)methyl)-4-methoxybenzo[d]isoxazol-3-yl)-3-((3aR,6aR)-5-((1-(3-(2,4-dioxotetrahydropyrimidin-1(2H)-yl)-1-ethyl-1H-indazol-6-yl)piperidin-4-yl)methyl)hexahydropyrrolo[3,4-c]pyrrol-2(1H)-yl)-2,6-dimethoxybenzenesulfonamide;
N-(6-((1H-Pyrazol-1-yl)methyl)-4-methoxybenzo[d]isoxazol-3-yl)-3-(11-((1-(3-(2,4-dioxotetrahydropyrimidin-1(2H)-yl)-1-methyl-1H-indazol-6-yl)piperidin-4-yl)methyl)-3,11-diazadispiro[5.1.58.16]tetradecan-3-yl)-2,6-dimethoxybenzenesulfonamide;
N-(6-((1H-Pyrazol-1-yl)methyl)-4-methoxybenzo[d]isoxazol-3-yl)-3-(7-((1-(3-(2,4-dioxotetrahydropyrimidin-1(2H)-yl)-1-ethyl-1H-indazol-6-yl)piperidin-4-yl)methyl)-2,7-diazaspiro[3.5]nonan-2-yl)-5-fluoro-2,6-dimethoxybenzenesulfonamide;
N-(6-((1H-Pyrazol-1-yl)methyl)-4-methoxybenzo[d]isoxazol-3-yl)-3-(7-((1-(4-(2,6-dioxopiperidin-3-yl)-2-methylphenyl)piperidin-4-yl)methyl)-2,7-diazaspiro[3.5]nonan-2-yl)-5-fluoro-2,6-dimethoxybenzenesulfonamide;
N-(6-((1H-Pyrazol-1-yl)methyl)-4-methoxybenzo[d]isoxazol-3-yl)-3-(7-((1-(4-(2,6-dioxopiperidin-3-yl)-3-fluorophenyl)piperidin-4-yl)methyl)-2,7-diazaspiro[3.5]nonan-2-yl)-2-methoxybenzenesulfonamide;
N-(6-((1H-Pyrazol-1-yl)methyl)-4-methoxybenzo[d]isoxazol-3-yl)-3-(7-((1-(4-(2,6-dioxopiperidin-3-yl)-3, 5-difluorophenyl)piperidin-4-yl)methyl)-2,7-diazaspiro[3.5]nonan-2-yl)-2-methoxybenzenesulfonamide;
N-(6-((1H-Pyrazol-1-yl)methyl)-4-methoxybenzo[d]isoxazol-3-yl)-3-(7-(2-(1-(4-(2,6-dioxopiperidin-3-yl)phenyl)piperidin-4-yl)ethyl)-2,7-diazaspiro[3.5]nonan-2-yl)-5-fluoro-2,6-dimethoxybenzenesulfonamide;
N-(6-((1H-Pyrazol-1-yl)methyl)-4-methoxybenzo[d]isoxazol-3-yl)-3-(7-(((3S)-1-(1-(2,6-dioxopiperidin-3-yl)-3-methyl-1H-indazol-5-yl)pyrrolidin-3-yl)methyl)-2,7-diazaspiro-[3.5]nonan-2-yl)-5-fluoro-2,6-dimethoxybenzenesulfonamide;
N-(6-((1H-Pyrazol-1-yl)methyl)-4-methoxybenzo[d]isoxazol-3-yl)-3-(7-(((3S)-1-(4-(2,6-dioxopiperidin-3-yl)phenyl)pyrrolidin-3-yl)methyl)-2,7-diazaspiro[3.5]nonan-2-yl)-5-fluoro-2,6-dimethoxybenzenesulfonamide;
N-(6-((1H-Pyrazol-1-yl)methyl)-4-methoxybenzo[d]isoxazol-3-yl)-3-(7-(((3R,4S)-1-(4-(2,6-dioxopiperidin-3-yl)phenyl)-3-fluoropiperidin-4-yl)methyl)-2,7-diazaspiro[3.5]nonan-2-yl)-5-fluoro-2,6-dimethoxybenzenesulfonamide;
N-(6-((1H-Pyrazol-1-yl)methyl)-4-methoxybenzo[d]isoxazol-3-yl)-3-(7-((1-(3-(2,4-dioxotetrahydropyrimidin-1(2H)-yl)-1-methyl-1H-pyrrolo[2,3-b]pyridin-6-yl)-4-fluoro-piperidin-4-yl)methyl)-2,7-diazaspiro[3.5]nonan-2-yl)-2-methoxybenzenesulfonamide;
3-[7-[[4-[4-(2,6-Dioxopiperidin-3-yl)phenyl]piperazin-1-yl]methyl]-2-azaspiro-[3.5]nonan-2-yl]-5-fluoro-2-methoxy-N-[4-methoxy-6-(pyrazol-1-ylmethyl)-1,2-benzoxazol-3-yl]benzenesulfonamide;
N-(6-((1H-Pyrazol-1-yl)methyl)-4-methoxybenzo[d]isoxazol-3-yl)-3-((1S,6S)-3-((1-(3-(2,4-dioxotetrahydropyrimidin-1(2H)-yl)-1-methyl-1H-indazol-6-yl)piperidin-4-yl)methyl)-3,7-diazabicyclo[4.2.0]octan-7-yl)-2-methoxy-6-methylbenzenesulfonamide;
N-(6-((1H-Pyrazol-1-yl)methyl)-4-methoxybenzo[d]isoxazol-3-yl)-3-(7-((1-(3-(2,6-dioxopiperidin-3-yl)-5-fluoro-1-methyl-1H-indazol-6-yl)piperidin-4-yl)methyl)-2,7-diazaspiro[3.5]nonan-2-yl)-5-fluoro-2,6-dimethoxybenzenesulfonamide;
N-(6-((1H-Pyrazol-1-yl)methyl)-4-methoxybenzo[d]isoxazol-3-yl)-3-((3R)-3-((4-(3-(2,6-dioxopiperidin-3-yl)-1-methyl-1H-indazol-6-yl)piperidin-1-yl)methyl)piperidin-1-yl)-2,6-dimethoxybenzenesulfonamide;
3-[6-[[4-[3-(2,6-Dioxopiperidin-3-yl)-1-methylindazol-6-yl]piperidin-1-yl]methyl]-2-pyridinyl]-2,6-dimethoxy-N-[4-methoxy-6-(pyrazol-1-ylmethyl)-1,2-benzoxazol-3-yl]benzenesulfonamide;
N-(6-((1H-Pyrazol-1-yl)methyl)-4-methoxybenzo[d]isoxazol-3-yl)-3-(4-((4-(3-(2,6-dioxopiperidin-3-yl)-1-methyl-1H-indazol-6-yl)piperidin-1-yl)methyl)pyridin-2-yl)-2,6-dimethoxybenzenesulfonamide;
N-(6-((1H-Pyrazol-1-yl)methyl)-4-methoxybenzo[d]isoxazol-3-yl)-3-(8-((1-(3-(2,4-dioxotetrahydropyrimidin-1(2H)-yl)-1-methyl-1H-indazol-6-yl)piperidin-4-yl)methyl)-8-azabicyclo[3.2.1]octan-3-yl)-5-fluoro-2,6-dimethoxybenzenesulfonamide;
N-(6-((1H-Pyrazol-1-yl)methyl)-4-methoxybenzo[d]isoxazol-3-yl)-3-(7-((1-(2-(2,6-dioxopiperidin-3-yl)-1-methyl-1H-pyrrolo[2,3-b]pyridin-6-yl)piperidin-4-yl)methyl)-2,7-diazaspiro[3.5]nonan-2-yl)-2-methoxybenzenesulfonamide;
N-(6-((1H-Pyrazol-1-yl)methyl)-4-methoxybenzo[d]isoxazol-3-yl)-3-(7-((1-(2-(2,6-dioxopiperidin-3-yl)-1-methyl-1H-pyrrolo[2,3-b]pyridin-6-yl)piperidin-4-yl)methyl)-2,7-diazaspiro[3.5]nonan-2-yl)-5-fluoro-2,6-dimethoxybenzenesulfonamide;
N-(6-((1H-Pyrazol-1-yl)methyl)-4-methoxybenzo[d]isoxazol-3-yl)-3-(7-((1-(5-(2,6-dioxopiperidin-3-yl)pyridin-2-yl)piperidin-4-yl)methyl)-2,7-diazaspiro[3.5]nonan-2-yl)-5-fluoro-2,6-dimethoxybenzenesulfonamide;
4-(4-((2-(3-(N-(6-((1H-Pyrazol-1-yl)methyl)-4-methoxybenzo[d]isoxazol-3-yl)sulfamoyl)-5-fluoro-2,4-dimethoxyphenyl)-2,7-diazaspiro[3.5]nonan-7-yl)methyl)-piperidin-1-yl)-N-(2,6-dioxopiperidin-3-yl)benzamide;
(R)—N-(6-((1H-Pyrazol-1-yl)methyl)-4-methoxybenzo[d]isoxazol-3-yl)-3-(7-((1-(4-(2,6-dioxopiperidin-3-yl-3,4,4,5,5-d5)phenyl)piperidin-4-yl)methyl)-2,7-diazaspiro[3.5]-nonan-2-yl)-5-fluoro-2,6-dimethoxybenzenesulfonamide;
(R)—N-(6-((1H-Pyrazol-1-yl)methyl)-4-methoxybenzo[d]isoxazol-3-yl)-3-(7-((1-(4-(2,6-dioxopiperidin-3-yl)phenyl)piperidin-4-yl)methyl)-2,7-diazaspiro[3.5]nonan-2-yl)-5-fluoro-2,6-dimethoxybenzenesulfonamide;
(S)—N-(6-((1H-Pyrazol-1-yl)methyl)-4-methoxybenzo[d]isoxazol-3-yl)-3-(7-((1-(4-(2,6-dioxopiperidin-3-yl)phenyl)piperidin-4-yl)methyl)-2,7-diazaspiro[3.5]nonan-2-yl)-5-fluoro-2,6-dimethoxybenzenesulfonamide;
N-(6-((1H-Pyrazol-1-yl)methyl)-4-methoxybenzo[d]isoxazol-3-yl)-3-(6-((1-(3-(2,6-dioxopiperidin-3-yl)-1-(methyl-d₃)-1H-pyrrolo[2,3-b]pyridin-6-yl)piperidin-4-yl)methyl)-5,6,7,8-tetrahydro-1,6-naphthyridin-2-yl)-5-fluoro-2,6-dimethoxybenzene-sulfonamide;
N-(6-((1H-Pyrazol-1-yl)methyl)-4-methoxybenzo[d]isoxazol-3-yl)-3-(6-((1-(3-(2,4-dioxotetrahydropyrimidin-1(2H)-yl)-1-(methyl-d₃)-1H-indazol-6-yl)piperidin-4-yl)methyl)-5,6,7,8-tetrahydro-1,6-naphthyridin-2-yl)-5-fluoro-2,6-dimethoxybenzenesulfonamide;
N-(6-((1H-Pyrazol-1-yl)methyl)-4-methoxybenzo[d]isoxazol-3-yl)-3-(7-((1-(3-(2,6-dioxopiperidin-3-yl)-1-(2,2,2-trifluoroethyl)-1H-indazol-6-yl)piperidin-4-yl)methyl)-2,7-diazaspiro[3.5]nonan-2-yl)-5-fluoro-2,6-dimethoxybenzenesulfonamide;
N-(6-((1H-Pyrazol-1-yl)methyl)-4-methoxybenzo[d]isoxazol-3-yl)-3-(2-((1-(3-(2,6-dioxopiperidin-3-yl)-1-methyl-1H-indazol-6-yl)piperidin-4-yl)methyl)-2,8-diazaspiro[4.5]-decan-8-yl)-5-fluoro-2,6-dimethoxybenzenesulfonamide;
N-(6-((1H-Pyrazol-1-yl)methyl)-4-methoxybenzo[d]isoxazol-3-yl)-3-(7-((1-(3-(2,6-dioxopiperidin-3-yl)-1-methyl-1H-indazol-6-yl)piperidin-4-yl)methyl)-5,6,7,8-tetrahydro-imidazo[1,2-a]pyrazin-3-yl)-2,6-dimethoxybenzenesulfonamide;
N-(6-((1H-Pyrazol-1-yl)methyl)-4-methoxybenzo[d]isoxazol-3-yl)-3-(7-(3-(2,6-dioxo-piperidin-3-yl)-1-methyl-1H-indazol-6-yl)-2,7-diazaspiro[3.5]nonan-2-yl)-2-methoxybenzene-sulfonamide;
or a pharmaceutically acceptable salt thereof.

It will be apparent that the compounds of the invention, including all subgenera described herein, may have multiple stereogenic centers. As a result, there exist multiple stereoisomers (enantiomers and diastereomers) of the compounds (and subgenera described herein). The present disclosure contemplates and encompasses each stereoisomer of any compound of encompassed by the disclosure as well as mixtures of said stereoisomers.

Pharmaceutically acceptable salts and solvates of the compounds of the disclosure (including all subgenera described herein) are also within the scope of the disclosure.

Isotopic variants of the compounds of the disclosure (including all subgenera described herein) are also contemplated by the present disclosure. Isotopes include those atoms have the same atomic number but different mass numbers. For example, isotopes of hydrogen are tritium and deuterium.

### Pharmaceutical Compositions and Methods of Administration

The subject pharmaceutical compositions are typically formulated to provide a therapeutically effective amount of a compound of the present disclosure as the active ingredient, or a pharmaceutically acceptable salt, ester, prodrug, solvate, hydrate or derivative thereof. Where desired, the pharmaceutical compositions contain pharmaceutically acceptable salt and/or coordination complex thereof, and one or more pharmaceutically acceptable excipients, carriers, including inert solid diluents and fillers, diluents, including sterile aqueous solution and various organic solvents, permeation enhancers, solubilizers and adjuvants.

The subject pharmaceutical compositions can be administered alone or in combination with one or more other agents, which are also typically administered in the form of pharmaceutical compositions. Where desired, the one or more compounds of the invention and other agent(s) may be mixed into a preparation or both components may be formulated into separate preparations to use them in combination separately or at the same time. In some embodiments, the concentration of one or more compounds provided in the pharmaceutical compositions of the present invention is less than 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% (or a number in the range defined by and including any two numbers above) w/w, w/v or v/v.

In some embodiments, the concentration of one or more compounds of the invention is greater than 90%, 80%, 70%, 60%, 50%, 40%, 30%, 20%, 19.75%, 19.50%, 19.25%, 19%, 18.75%, 18.50%, 18.25% 18%, 17.75%, 17.50%, 17.25% 17%, 16.75%, 16.50%, 16.25%, 16%, 15.75%, 15.50%, 15.25% 15%, 14.75%, 14.50%, 14.25% 14%, 13.75%, 13.50%, 13.25%, 13%, 12.75%, 12.50%, 12.25%, 12%, 11.75%, 11.50%, 11.25% 11%, 10.75%, 10.50%, 10.25% 10%, 9.75%, 9.50%, 9.25%, 9%, 8.75%, 8.50%, 8.25% 8%, 7.75%, 7.50%, 7.25%, 7%, 6.75%, 6.50%, 6.25%, 6%, 5.75%, 5.50%, 5.25%, 5%, 4.75%, 4.50%, 4.25%, 4%, 3.75%, 3.50%, 3.25%, 3%, 2.75%, 2.50%, 2.25%, 2%, 1.75%, 1.50%, 1.25%, 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% (or a number in the range defined by and including any two numbers above) w/w, w/v, or v/v.

In some embodiments, the concentration of one or more compounds of the invention is in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, approximately 1% to approximately 10% w/w, w/v or v/v.

In some embodiments, the concentration of one or more compounds of the invention is in the range from approximately 0.001% to approximately 10%, approximately 0.01% to approximately 5%, approximately 0.02% to approximately 4.5%, approximately 0.03% to approximately 4%, approximately 0.04% to approximately 3.5%, approximately 0.05% to approximately 3%, approximately 0.06% to approximately 2.5%, approximately 0.07% to approximately 2%, approximately 0.08% to approximately 1.5%, approximately 0.09% to approximately 1%, approximately 0.1% to approximately 0.9% w/w, w/v or v/v.

In some embodiments, the amount of one or more compounds of the invention is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g (or a number in the range defined by and including any two numbers above).

In some embodiments, the amount of one or more compounds of the invention is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, , 0.15 g, 0.2 g, , 0.25 g, 0.3 g, , 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g (or a number in the range defined by and including any two numbers above).

In some embodiments, the amount of one or more compounds of the invention is in the range of 0.0001-10 g, 0.0005-9 g, 0.001-8 g, 0.005-7 g, 0.01-6 g, 0.05-5 g, 0.1-4 g, 0.5-4 g, or 1-3 g.

The compounds according to the invention are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. An exemplary dosage is 10 to 30 mg per day. The exact dosage will depend upon the route of administration, the form in which the compound is administered, the subject to be treated, the body weight of the subject to be treated, and the preference and experience of the attending physician.

A pharmaceutical composition of the invention typically contains an active ingredient (e.g., a compound of the disclosure) of the present invention or a pharmaceutically acceptable salt and/or coordination complex thereof, and one or more pharmaceutically acceptable excipients, carriers, including but not limited to inert solid diluents and fillers, diluents, sterile aqueous solution and various organic solvents, permeation enhancers, solubilizers and adjuvants.

Described below are non-limiting exemplary pharmaceutical compositions and methods for preparing the same.

### Pharmaceutical Compositions for Oral Administration.

In some embodiments, the invention provides a pharmaceutical composition for oral administration containing a compound of the invention, and a pharmaceutical excipient suitable for oral administration.

In some embodiments, the invention provides a solid pharmaceutical composition for oral administration containing: (i) an effective amount of a compound of the invention; optionally (ii) an effective amount of a second agent; and (iii) a pharmaceutical excipient suitable for oral administration. In some embodiments, the composition further contains: (iv) an effective amount of a third agent.

In some embodiments, the pharmaceutical composition may be a liquid pharmaceutical composition suitable for oral consumption. Pharmaceutical compositions of the invention suitable for oral administration can be presented as discrete dosage forms, such as capsules, cachets, or tablets, or liquids or aerosol sprays each containing a predetermined amount of an active ingredient as a powder or in granules, a solution, or a suspension in an aqueous or non-aqueous liquid, an oil-in-water emulsion, or a water-in-oil liquid emulsion. Such dosage forms can be prepared by any of the methods of pharmacy, but all methods include the step of bringing the active ingredient into association with the carrier, which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

This invention further encompasses anhydrous pharmaceutical compositions and dosage forms comprising an active ingredient, since water can facilitate the degradation of some compounds. For example, water may be added (e.g., 5%) in the pharmaceutical arts as a means of simulating long-term storage in order to determine characteristics such as shelf-life or the stability of formulations over time. Anhydrous pharmaceutical compositions and dosage forms of the invention can be prepared using anhydrous or low moisture containing ingredients and low moisture or low humidity conditions. Pharmaceutical compositions and dosage forms of the invention which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

An active ingredient can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants may be used in the compositions of the invention to provide tablets that disintegrate when exposed to an aqueous environment. Too much of a disintegrant may produce tablets which may disintegrate in the bottle. Too little may be insufficient for disintegration to occur and may thus alter the rate and extent of release of the active ingredient(s) from the dosage form. Thus, a sufficient amount of disintegrant that is neither too little nor too much to detrimentally alter the release of the active ingredient(s) may be used to form the dosage forms of the compounds disclosed herein. The amount of disintegrant used may vary based upon the type of formulation and mode of administration, and may be readily discernible to those of ordinary skill in the art. About 0.5 to about 15 weight percent of disintegrant, or about 1 to about 5 weight percent of disintegrant, may be used in the pharmaceutical composition. Disintegrants that can be used to form pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, or mixtures thereof. Additional lubricants include, for example, a syloid silica gel, a coagulated aerosol of synthetic silica, or mixtures thereof. A lubricant can optionally be added, in an amount of less than about 1 weight percent of the pharmaceutical composition.

When aqueous suspensions and/or elixirs are desired for oral administration, the active ingredient therein may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactant which can be used to form pharmaceutical compositions and dosage forms of the invention include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions.

Hydrophilic surfactants are generally considered to be those compounds having an HLB value greater than about 10, as well as anionic, cationic, or zwitterionic compounds for which the HLB scale is not generally applicable. Similarly, lipophilic (e.g., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acyl lactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidyl-choline, phosphatidylethanolamine, phosphatidylglycerol, phosphatidic acid, phosphatidyl-serine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but are not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof, polyoxyethylated vitamins and derivatives thereof, polyoxyethylene-polyoxypropylene block copolymers; and mixtures thereof, polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-lOoleate, Tween 40, Tween 60, sucrose monostearate, sucrose mono laurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In one embodiment, the composition may include a solubilizer to ensure good solubilization and/or dissolution of the compound of the present invention and to minimize precipitation of the compound of the present invention. This can be especially important for compositions for non-oral use, e.g., compositions for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, ε-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, ε-caprolactone and isomers thereof, δ-valerolactone and isomers thereof, β-butyrolactone and isomers thereof, and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a subject using conventional techniques, such as distillation or evaporation. Thus, if present, the solubilizer can be in a weight ratio of 10%, 25% o, 50%), 100% o, or up to about 200%>by weight, based on the combined weight of the drug, and other excipients. If desired, very small amounts of solubilizer may also be used, such as 5%>, 2%>, 1%) or even less. Typically, the solubilizer may be present in an amount of about 1%>to about 100%, more typically about 5%>to about 25%>by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals, alkaline earth metals, and the like. Example may include, but not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid and the like.

### Pharmaceutical Compositions for Injection.

In some embodiments, the invention provides a pharmaceutical composition for injection containing a compound of the present invention and a pharmaceutical excipient suitable for injection. Components and amounts of agents in the compositions are as described herein.

The forms in which the novel compositions of the present invention may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating the compound of the present invention in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

### Pharmaceutical Compositions for Topical (e.g., Transdermal) Delivery.

In some embodiments, the invention provides a pharmaceutical composition for transdermal delivery containing a compound of the present invention and a pharmaceutical excipient suitable for transdermal delivery.

Compositions of the present invention can be formulated into preparations in solid, semisolid, or liquid forms suitable for local or topical administration, such as gels, water soluble jellies, creams, lotions, suspensions, foams, powders, slurries, ointments, solutions, oils, pastes, suppositories, sprays, emulsions, saline solutions, dimethylsulfoxide (DMSO)-based solutions. In general, carriers with higher densities are capable of providing an area with a prolonged exposure to the active ingredients. In contrast, a solution formulation may provide more immediate exposure of the active ingredient to the chosen area.

The pharmaceutical compositions also may comprise suitable solid or gel phase carriers or excipients, which are compounds that allow increased penetration of, or assist in the delivery of, therapeutic molecules across the stratum corneum permeability barrier of the skin. There are many of these penetration-enhancing molecules known to those trained in the art of topical formulation.

Examples of such carriers and excipients include, but are not limited to, humectants (e.g., urea), glycols (e.g., propylene glycol), alcohols (e.g., ethanol), fatty acids (e.g., oleic acid), surfactants (e.g., isopropyl myristate and sodium lauryl sulfate), pyrrolidones, glycerol monolaurate, sulfoxides, terpenes (e.g., menthol), amines, amides, alkanes, alkanols, water, calcium carbonate, calcium phosphate, various sugars, starches, cellulose derivatives, gelatin, and polymers such as polyethylene glycols.

Another exemplary formulation for use in the methods of the present invention employs transdermal delivery devices (“patches”). Such transdermal patches may be used to provide continuous or discontinuous infusion of a compound of the present invention in controlled amounts, either with or without another agent.

The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

### Pharmaceutical Compositions for Inhalation.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices that deliver the formulation in an appropriate manner.

### Other Pharmaceutical Compositions.

Pharmaceutical compositions may also be prepared from compositions described herein and one or more pharmaceutically acceptable excipients suitable for sublingual, buccal, rectal, intraosseous, intraocular, intranasal, epidural, or intraspinal administration. Preparations for such pharmaceutical compositions are well-known in the art. See, e.g., Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 20037ybg; Goodman and Gilman, eds., The Pharmacological Basis of Therapeutics, Tenth Edition, McGraw Hill, 2001; Remingtons Pharmaceutical Sciences, 20th Ed., Lippincott Williams & Wilkins., 2000; Martindale, The Extra Pharmacopoeia, Thirty-Second Edition (The Pharmaceutical Press, London, 1999); all of which are incorporated by reference herein in their entirety.

Administration of the compounds or pharmaceutical composition of the present invention can be effected by any method that enables delivery of the compounds to the site of action. These methods include oral routes, intraduodenal routes, parenteral injection (including intravenous, intraarterial, subcutaneous, intramuscular, intravascular, intraperitoneal or infusion), topical (e.g. transdermal application), rectal administration, via local delivery by catheter or stent or through inhalation. Compounds can also be administered intraadiposally or intrathecally.

In some embodiments, the compounds or pharmaceutical composition of the present invention are administered by intravenous injection.

The amount of the compound administered will be dependent on the subject being treated, the severity of the disorder or condition, the rate of administration, the disposition of the compound and the discretion of the prescribing physician. However, an effective dosage is in the range of about 0.001 to about 100 mg per kg body weight per day, preferably about 1 to about 35 mg/kg/day, in single or divided doses. For a 70 kg human, this would amount to about 0.05 to 7 g/day, preferably about 0.05 to about 2.5 g/day. In some instances, dosage levels below the lower limit of the aforesaid range may be more than adequate, while in other cases still larger doses may be employed without causing any harmful side effect, e.g. by dividing such larger doses into several small doses for administration throughout the day.

In some embodiments, a compound of the invention is administered in a single dose. Typically, such administration will be by injection, e.g., intravenous injection, in order to introduce the agent quickly. However, other routes may be used as appropriate. A single dose of a compound of the invention may also be used for treatment of an acute condition.

In some embodiments, a compound of the invention is administered in multiple doses. Dosing may be about once, twice, three times, four times, five times, six times, or more than six times per day. Dosing may be about once a month, once every two weeks, once a week, or once every other day. In another embodiment a compound of the invention and another agent are administered together about once per day to about 6 times per day. In another embodiment the administration of a compound of the invention and an agent continues for less than about 7 days. In yet another embodiment the administration continues for more than about 6, 10, 14, 28 days, two months, six months, or one year. In some cases, continuous dosing is achieved and maintained as long as necessary.

Administration of the compounds of the invention may continue as long as necessary. In some embodiments, a compound of the invention is administered for more than 1, 2, 3, 4, 5, 6, 7, 14, or 28 days. In some embodiments, a compound of the invention is administered for less than 28, 14, 7, 6, 5, 4, 3, 2, or 1 day. In some embodiments, a compound of the invention is administered chronically on an ongoing basis, e.g., for the treatment of chronic effects.

An effective amount of a compound of the invention may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, or as an inhalant.

The compositions of the invention may also be delivered via an impregnated or coated device such as a stent, for example, or an artery-inserted cylindrical polymer. Such a method of administration may, for example, aid in the prevention or amelioration of restenosis following procedures such as balloon angioplasty. Without being bound by theory, compounds of the invention may slow or inhibit the migration and proliferation of smooth muscle cells in the arterial wall which contribute to restenosis. A compound of the invention may be administered, for example, by local delivery from the struts of a stent, from a stent graft, from grafts, or from the cover or sheath of a stent. In some embodiments, a compound of the invention is admixed with a matrix. Such a matrix may be a polymeric matrix, and may serve to bond the compound to the stent. Polymeric matrices suitable for such use, include, for example, lactone-based polyesters or copolyesters such as polylactide, polycaprolactonglycolide, polyorthoesters, polyanhydrides, polyaminoacids, polysaccharides, polyphosphazenes, poly (ether-ester) copolymers (e.g. PEO-PLLA); polydimethylsiloxane, poly(ethylene-vinylacetate), acrylate-based polymers or copolymers (e.g. polyhydroxyethyl methylmethacrylate, polyvinyl pyrrolidinone), fluorinated polymers such as polytetrafluoroethylene and cellulose esters. Suitable matrices may be nondegrading or may degrade with time, releasing the compound or compounds. Compounds of the invention may be applied to the surface of the stent by various methods such as dip/spin coating, spray coating, dip-coating, and/or brush-coating. The compounds may be applied in a solvent and the solvent may be allowed to evaporate, thus forming a layer of compound onto the stent. Alternatively, the compound may be located in the body of the stent or graft, for example in microchannels or micropores. When implanted, the compound diffuses out of the body of the stent to contact the arterial wall. Such stents may be prepared by dipping a stent manufactured to contain such micropores or microchannels into a solution of the compound of the invention in a suitable solvent, followed by evaporation of the solvent. Excess drug on the surface of the stent may be removed via an additional brief solvent wash. In yet other embodiments, compounds of the invention may be covalently linked to a stent or graft. To link compounds of the invention to the stent or graft, a covalent linker may be used which degrades in vivo, leading to the release of the compound from the stent. Any bio-labile linkage may be used for such a purpose, such as ester, amide or anhydride linkages. Compounds of the invention may additionally be administered intravascularly from a balloon used during angioplasty. Extravascular administration of the compounds via the pericard or via advential application of formulations of the invention may also be performed to decrease restenosis.

A variety of stent devices which may be used as described are disclosed, for example, in the following references, all of which are hereby incorporated by reference: U.S. Pat. Nos. 5,451,233; 5,040,548; 5,061,273; 5,496,346; 5,292,331; 5,674,278; 3,657,744; 4,739,762; 5,195,984; 5,292,331; 5,674,278; 5,879,382; 6,344,053.

The compounds of the invention may be administered in dosages. It is known in the art that due to intersubject variability in compound pharmacokinetics, individualization of dosing regimen is necessary for optimal therapy. Dosing for a compound of the invention may be found by routine experimentation in light of the instant disclosure.

When a compound of the invention is administered in a composition that comprises one or more agents, and the agent has a shorter half-life than the compound of the invention unit dose forms of the agent and the compound of the invention may be adjusted accordingly. The subject pharmaceutical composition may, for example, be in a form suitable for oral administration as a tablet, capsule, pill, powder, sustained release formulations, solution, suspension, for parenteral injection as a sterile solution, suspension or emulsion, for topical administration as an ointment or cream or for rectal administration as a suppository. The pharmaceutical composition may be in unit dosage forms suitable for single administration of precise dosages. The pharmaceutical composition will include a conventional pharmaceutical carrier or excipient and a compound according to the invention as an active ingredient. In addition, it may include other medicinal or pharmaceutical agents, carriers, adjuvants, etc. Exemplary parenteral administration forms include solutions or suspensions of active compound in sterile aqueous solutions, for example, aqueous propylene glycol or dextrose solutions. Such dosage forms can be suitably buffered, if desired.

### METHODS OF USE

The method typically comprises administering to a subject a therapeutically effective amount of a compound of the invention. The therapeutically effective amount of the subject combination of compounds may vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of proliferation or down-regulation of activity of a target protein. The specific dose will vary depending on the particular compounds chosen, the dosing regimen to be followed, whether it is administered in combination with other compounds, timing of administration, the tissue to which it is administered, and the physical delivery system in which it is carried.

In certain embodiment, the present invention provides a pharmaceutical composition comprising a compound of bispecific formula, or pharmaceutically acceptable salt thereof.

In certain embodiment, the present invention provides a pharmaceutical composition comprising a compound of bispecific formula for use in degrading a target protein in a cell.

In certain embodiment, a method of degrading a target protein comprising administering to a cell therapeutically effective amount of a bispecific compound, or pharmaceutically acceptable salt, wherein the compound is effective for degrading the target protein.

“Abnormal cell growth” or “cancer” as used herein, unless otherwise indicated, refers to cell growth that is independent of normal regulatory mechanisms (e.g., loss of contact inhibition). This includes the abnormal growth of: (1) tumor cells (tumors) that proliferate by expressing a mutated tyrosine kinase or overexpression of a receptor tyrosine kinase; (2) benign and malignant cells of other proliferative diseases in which aberrant tyrosine kinase activation occurs; (3) any tumors that proliferate by receptor tyrosine kinases; (4) any tumors that proliferate by aberrant serine/threonine kinase activation; (5) benign and malignant cells of other proliferative diseases in which aberrant serine/threonine kinase activation occurs; (6) any tumors that proliferate by aberrant signaling, metabolic, epigenetic and transcriptional mechanism; and (7) benign and malignant cells of other proliferative diseases in which aberrant signaling, metabolic, epigenetic and transcriptional mechanism.

For convenience, certain well-known abbreviations, may be used herein, including: estrogen receptor positive (ER+), human epidermal growth factor receptor 2 negative (HER2−), non-small cell lung cancer (NSCLC) and castration resistant prostate cancer (CRPC).

Further embodiments relate to methods of treating abnormal cell growth in a patient. Additional embodiments relate to a method of treating abnormal cell growth in a patient comprising administering to the patient an amount of a compound described herein that is effective in treating abnormal cell growth.

In other embodiments, the abnormal cell growth is cancer.

In some embodiments, the cancer is selected from the group consisting of lung cancer, mesothelioma, bone cancer, pancreatic cancer, skin cancer, cancer of the head or neck, cutaneous or intraocular melanoma, uterine cancer, ovarian cancer, rectal cancer, cancer of the anal region, stomach cancer, hepatic carcinoma, colon cancer, breast cancer, uterine cancer, carcinoma of the fallopian tubes, carcinoma of the endometrium, carcinoma of the cervix, carcinoma of the vagina, carcinoma of the vulva, Hodgkin's disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, hematology malignancy, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, glioblastoma, brain stem glioma, pituitary adenoma, or a combination of two or more of the foregoing cancers.

Additional embodiments relate to methods of treating solid tumors in a patient. Some embodiments relate to the treatment of solid tumors in a patient comprising administering to the patient an amount of a compound described herein that is effective in treating the solid tumor.

In one embodiment, the solid tumor is breast, lung, colon, brain, prostate, stomach, pancreatic, ovarian, melanoma, endocrine, uterine, testicular, or bladder.

In one embodiment, the solid tumor is breast, lung, prostate, pancreatic, or ovarian.

In one embodiment, the cancer is breast cancer.

In one embodiment, the breast cancer is ER+ breast cancer.

In one embodiment, the breast cancer is ER+ HER2− breast cancer.

In one embodiment, the breast cancer is locally advanced or metastatic ER+ HER2− breast cancer.

In one embodiment, the lung cancer is non-small cell lung cancer.

In one embodiment, the lung cancer is locally advanced or metastatic non-small cell lung cancer.

In one embodiment, the prostate cancer is castration resistant prostate cancer. In one embodiment, the prostate cancer is locally advanced or metastatic castration resistant prostate cancer.

Additional embodiments relate to methods of treating hematologic tumors in a patient. Some embodiments relate to the treatment of hematologic tumors in a patient comprising administering to the patient an amount of a compound described herein that is effective in treating the hematologic tumor.

In one embodiment, the hematologic tumor is leukemia, lymphoma or multiple myeloma.

In one embodiment, the hematologic tumor is leukemia or lymphoma.

Additional embodiments relate to methods of treating cancer in a patient comprising administering to the patient an amount of a compound described herein that is effective in treating cancer. In one embodiment, the cancer is breast, lung, colon, brain, prostate, stomach, pancreatic, ovarian, melanoma, endocrine, uterine, testicular, bladder, or hematologic.

In one embodiment, the cancer is breast, lung, prostate, pancreatic, ovarian, or hematologic.

In one embodiment, the cancer is breast, lung, prostate, pancreatic, or ovarian. In one embodiment, the cancer is breast cancer.

In one embodiment, the breast cancer is ER+ breast cancer.

In one embodiment, the breast cancer is ER+ HER2− breast cancer.

In one embodiment, the breast cancer is locally advanced or metastatic ER+ HER2− breast cancer.

In one embodiment, the lung cancer is non-small cell lung cancer.

In one embodiment, the lung cancer is locally advanced or metastatic non-small cell lung cancer.

In one embodiment, the prostate cancer is castration resistant prostate cancer. In one embodiment, the prostate cancer is locally advanced or metastatic castration resistant prostate cancer.

In one embodiment, the cancer is hematologic.

In one embodiment, the hematologic tumor is leukemia or lymphoma.

Further embodiments relate to methods of treating cancer in a patient which comprises administering to the patient an amount of a compound described herein that is effective in treating cancer in combination with an anti-tumor agent selected from the group consisting of mitotic inhibitors, alkylating agents, anti-metabolites, intercalating antibiotics, growth factor inhibitors, radiation, cell cycle inhibitors, enzymes, topoisomerase inhibitors, biological response modifiers, antibodies, cytotoxics, anti-hormones, and anti-androgens.

More embodiments relate to pharmaceutical compositions for treating cancer in a patient comprising an amount of a compound described herein that is effective in treating cancer, and a pharmaceutically acceptable carrier.

Additional embodiments relate to a method of treating cancer in a patient, and in particular a human, comprising administering to the patient an amount of a compound described herein, or a pharmaceutically acceptable salt, solvate, hydrate or prodrug thereof, that is effective in treating cancer. In one embodiment of this method, the cancer, includes, but is not limited to, lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers.

In one embodiment the method comprises comprising administering to a patient an amount of a compound described herein that is effective in treating said cancer solid tumor. In one preferred embodiment the solid tumor is breast, lung, colon, brain, prostate, stomach, pancreatic, ovarian, skin (melanoma), endocrine, uterine, testicular, and bladder cancer.

In another embodiment of said method, said cancer is a benign proliferative disease, including, but not limited to, psoriasis, benign prostatic hypertrophy or restenosis.

Some embodiments relate to a method of treating cancer in a patient which comprises administering to said patient an amount of a compound described herein, or a pharmaceutically acceptable salt, solvate, hydrate or prodrug thereof, that is effective in treating cancer in combination with an anti-tumor agent selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, antibodies, cytotoxics, anti-hormones, and anti-androgens.

Additional embodiments relate to a pharmaceutical composition for treating cancer in a patient, and in particular a human, comprising an amount of a compound described herein, or a pharmaceutically acceptable salt, solvate, hydrate or prodrug thereof, that is effective in treating cancer, and a pharmaceutically acceptable carrier. In one embodiment of said composition, the cancer, includes, but not limited to, lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers. In another embodiment of said pharmaceutical composition, said abnormal cell growth is a benign proliferative disease, including, but not limited to, psoriasis, benign prostatic hypertrophy or restinosis.

Further embodiments relate to a method of treating cancer in a patient which comprises administering to said patient an amount of a compound described herein, or a pharmaceutically acceptable salt, solvate, or hydrate thereof, that is effective in treating cancer in combination with another anti-tumor agent selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, antibodies, cytotoxics, anti-hormones, and anti-androgens. Some embodiments contemplate a pharmaceutical composition for treating abnormal cell growth wherein the composition includes a compound described herein, or a pharmaceutically acceptable salt, solvate, or hydrate thereof, that is effective in treating abnormal cell growth, and another anti-tumor agent selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, antibodies, cytotoxics, anti-hormones, and anti-androgens.

Yet more embodiments relate to a method of treating a disorder associated with angiogenesis in a patient, including a human, comprising administering to said patient an amount of a compound described herein, as defined above, or a pharmaceutically acceptable salt, solvate, hydrate or prodrug thereof, that is effective in treating said disorder in combination with one or more anti-tumor agents listed above. Such disorders include cancerous tumors such as melanoma; ocular disorders such as age-related macular degeneration, presumed ocular histoplasmosis syndrome, and retinal neovascularization from proliferative diabetic retinopathy; rheumatoid arthritis; bone loss disorders such as osteoporosis, Paget's disease, humoral hypercalcemia of malignancy, hypercalcemia from tumors metastatic to bone, and osteoporosis induced by glucocorticoid treatment; coronary restenosis; and certain microbial infections including those associated with microbial pathogens selected from adenovirus, hantaviruses, Borrelia burgdorferi, Yersinia spp., Bordetella pertussis, and group A Streptococcus.

Compounds of the disclosure, as well as pharmaceutical compositions comprising them, can be administered to treat any of the described diseases, alone or in combination with a medical therapy. Medical therapies include, for example, surgery and radiotherapy (e.g., gamma-radiation, neutron beam radiotherapy, electron beam radiotherapy, proton therapy, brachytherapy, systemic radioactive isotopes).

In other aspects, compounds of the disclosure, as well as pharmaceutical compositions comprising them, can be administered to treat any of the described diseases, alone or in combination with one or more other agents.

In other methods, the compounds of the disclosure, as well as pharmaceutical compositions comprising them, can be administered in combination with agonists of nuclear receptors agents.

In other methods, the compounds of the disclosure, as well as pharmaceutical compositions comprising them, can be administered in combination with antagonists of nuclear receptors agents.

In other methods, the compounds of the disclosure, as well as pharmaceutical compositions comprising them, can be administered in combination with an anti-proliferative agent.

### Combination Therapies

For treating cancer and other proliferative diseases, the compounds of the invention can be used in combination with chemotherapeutic agents, agonists or antagonists of nuclear receptors, or other anti-proliferative agents. The compounds of the invention can also be used in combination with a medical therapy such as surgery or radiotherapy, e.g., gamma-radiation, neutron beam radiotherapy, electron beam radiotherapy, proton therapy, brachytherapy, and systemic radioactive isotopes. Examples of suitable chemotherapeutic agents include any of: abarelix, aldesleukin, alemtuzumab, alitretinoin, allopurinol, all-trans retinoic acid, altretamine, anastrozole, arsenic trioxide, asparaginase, azacitidine, bendamustine, bevacizumab, bexarotene, bleomycin, bortezombi, bortezomib, busulfan intravenous, busulfan oral, calusterone, capecitabine, carboplatin, carmustine, cetuximab, chlorambucil, cisplatin, cladribine, clofarabine, cyclophosphamide, cytarabine, dacarbazine, dactinomycin, dalteparin sodium, dasatinib, daunorubicin, decitabine, denileukin, denileukin diftitox, dexrazoxane, docetaxel, doxorubicin, dromostanolone propionate, eculizumab, epirubicin, erlotinib, estramustine, etoposide phosphate, etoposide, exemestane, fentanyl citrate, filgrastim, floxuridine, fludarabine, fluorouracil, fulvestrant, gefitinib, gemcitabine, gemtuzumab ozogamicin, goserelin acetate, histrelin acetate, ibritumomab tiuxetan, idarubicin, ifosfamide, imatinib mesylate, interferon alfa 2a, irinotecan, lapatinib ditosylate, lenalidomide, letrozole, leucovorin, leuprolide acetate, levamisole, lomustine, meclorethamine, megestrol acetate, melphalan, mercaptopurine, methotrexate, methoxsalen, mitomycin C, mitotane, mitoxantrone, nandrolone phenpropionate, nelarabine, nofetumomab, oxaliplatin, paclitaxel, pamidronate, panobinostat, panitumumab, pegaspargase, pegfilgrastim, pemetrexed disodium, pentostatin, pipobroman, plicamycin, procarbazine, quinacrine, rasburicase, rituximab, ruxolitinib, sorafenib, streptozocin, sunitinib, sunitinib maleate, tamoxifen, temozolomide, teniposide, testolactone, thalidomide, thioguanine, thiotepa, topotecan, toremifene, tositumomab, trastuzumab, tretinoin, uracil mustard, valrubicin, vinblastine, vincristine, vinorelbine, vorinstat and zoledronate.

Some embodiments relate to a method of (and to a pharmaceutical composition for) treating cancer in a patient which comprise an amount of a compound described herein, or a pharmaceutically acceptable salt, solvate, or hydrate thereof, in combination with an amount of one or more substances selected from anti-angiogenesis agents, signal transduction inhibitors (e.g., inhibiting the means by which regulatory molecules that govern the fundamental processes of cell growth, differentiation, and survival communicated within the cell), and antiproliferative agents, which amounts are together effective in treating said abnormal cell growth.

Anti-angiogenesis agents, such as MMP-2 (matrix-metalloprotienase 2) inhibitors, MMP-9 (matrix-metalloprotienase 9) inhibitors, and COX-II (cyclooxygenase II) inhibitors, can be used in conjunction with a compound described herein in the methods and pharmaceutical compositions described herein.

Tyrosine kinase inhibitors can also be combined with a compound described herein.

VEGF inhibitors, for example, sutent and axitinib, can also be combined with a compound described herein.

ErbB2 receptor inhibitors may be administered in combination with a compound described herein. Various other compounds, such as styrene derivatives, have also been shown to possess tyrosine kinase inhibitory properties, and some of tyrosine kinase inhibitors have been identified as erbB2 receptor inhibitors.

Epidermal growth factor receptor (EGFR) inhibitors may be administered in combination with a compound of the present invention. PI3K inhibitors, such as PI3K alpha or PI3K beta inhibitors, may be administered in combination with a compound of the present invention.

Mammalian target of rapamycin (mTOR) inhibitors may be administered in combination with a compound of the present invention.

c-Met inhibitors may be administered in combination with a compound of the present invention.

CDK inhibitors may be administered in combination with a compound of the present invention.

MEK inhibitors may be administered in combination with a compound of the present invention.

PARP inhibitors may be administered in combination with a compound of the present invention.

JAK inhibitors may be administered in combination with a compound of the present invention.

An antagonist of a Programmed Death 1 protein (PD-1) may be administered in combination with a compound of the present invention.

An antagonist of Programmed Death-Ligand 1 (PD-L1) may be administered in combination with a compound of the present invention.

Other antiproliferative agents that may be used with the compounds described herein include inhibitors of the enzyme farnesyl protein transferase and inhibitors of the receptor tyrosine kinase PDGFr.

A compound described herein may also be used with other agents useful in treating abnormal cell growth or cancer, including, but not limited to, agents capable of enhancing antitumor immune responses, such as CTLA4 (cytotoxic lymphocyte antigen 4) antibodies, and other agents capable of blocking CTLA4; and anti-proliferative agents such as other farnesyl protein transferase inhibitors, for example the farnesyl protein transferase.

A compound described herein may be applied as a sole therapy or may involve one or more other anti-tumor substances, for example those selected from, for example, mitotic inhibitors, alkylating agents, anti-metabolites, growth factor inhibitors, cell cycle inhibitors, intercalating antibiotics, enzymes, and anti-hormones.

The compounds described herein may be used alone or in combination with one or more of a variety of anti-cancer agents or supportive care agents. For example, the compounds described herein may be used with cytotoxic agents. Some embodiments also contemplate the use of the compounds described herein together with hormonal therapy. Further, some embodiments provide a compound described herein alone or in combination with one or more supportive care products, e.g., a product selected from the group consisting of Filgrastim (Neupogen), ondansetron (Zofran), Fragmin, Procrit, Aloxi, Emend, or combinations thereof. Such conjoint treatment may be achieved by way of the simultaneous, sequential or separate dosing of the individual components of the treatment.

The compounds described herein may be used with antitumor agents, alkylating agents, antimetabolites, antibiotics, plant-derived antitumor agents, camptothecin derivatives, tyrosine kinase inhibitors, antibodies, interferons, and/or biological response modifiers. In this regard, the following is a non-limiting list of examples of secondary agents that may be used with the compounds described herein.

### ASPECTS OF THE INVENTION

In a first aspect of the invention, provided is a compound of Formula (I)

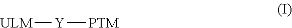

or a pharmaceutically acceptable salt or solvate thereof,
wherein Y-PTM is a moiety of Formula IA:

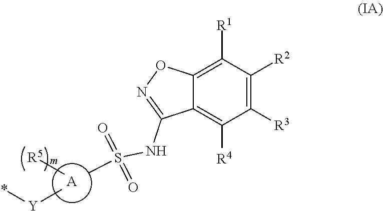

wherein
   Y is a covalent bond, or chemical moiety that links PTM and ULM;
   * is a point of attachment to ULM;
   Ring A is a C₆-C₁₀ membered aryl group or a 5-10 membered heteroaryl group;
   R¹ is H or 5-6 membered heteroaryl optionally substituted by methyl;
   R² is H, C₁₋₄alk-O—C₁₋₄alkyl, or —(C(R⁸)₂)ₙ-(5-9 membered heteroaryl) optionally substituted by halogen, C₁-C₃ alkyl, —CH₂OH, or —OH;
   R³ is H or halogen, C₁-C₄ alkyl, cyclopropyl, haloalkyl, C₁-C₄ alkoxy, or haloalkoxy;
   R⁴ is H or halogen, C₁-C₄ alkyl, cyclopropyl, C₁-C₄ alkoxy, or —O-cyclopropyl;
   each R⁵ is independently H, halogen, oxo, —OH, —CN, —NO₂, —C₁-C₆alkyl, —C₂-C₆alkenyl, —C₂-C₆alkynyl, C₀-C₁alk-aryl, C₀-C₁alk-heteroaryl, cycloalkyl, cycloalkenyl, heterocycloalkyl, or heterocycloalkenyl, —OR^{a}, —SR^{a}, —NR^{c}R^{d}, —NR^{a}R^{c}, —C(O)R^{b}, —OC(O)R^{a}, —C(O)OR^{a}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{d})(OR^{c}) or —S(O)₂R^{b};
   each R^{a} is independently H, —C(O)R^{b}, —C(O)OR^{c}, —C(O)NR^{c}R^{d}, —C(═NR^{b})NR^{b}R^{c}, —C(═NOR^{b})R^{b}R^{c}, —C(═NCN)NR^{b}R^{c}, —P(OR^{c})₂, —P(O)R^{c}R^{b}, —P(O)OR^{c}OR^{b}, —S(O)R^{b}, —S(O)NR^{c}R^{d}, —S(O)₂R^{b}, —S(O)₂NR^{c}R^{d}, SiR^{b}₃, —C₁-C₁₀alkyl, —C₁-C₁₀ haloalkyl, —C₂-C₁₀ alkenyl, —C₂-C₁₀ alkynyl, aryl, cycloalkyl, cycloalkenyl, heteroaryl, heterocycloalkyl, or heterocycloalkenyl;
   each R^{b}, is independently H, —C₁-C₆ alkyl, —C₁-C₆ haloalkyl, —C₂-C₆ alkenyl, —C₂-C₆ alkynyl, aryl, cycloalkyl, cycloalkenyl, heteroaryl, heterocycloalkyl, or heterocycloalkenyl;
   each R^{c} or R^{d} is independently H, —C₁-C₁₀ alkyl, —C₂-C₆ alkenyl, —C₂-C₆ alkynyl, —OC₁-C₆alkyl, —O-cycloalkyl, aryl, heteroaryl, cycloalkyl, cycloalkenyl, heterocycloalkyl, or heterocycloalkenyl; or
   R and R^{d}, together with the atom to which they are both attached, form a monocyclic or multicyclic heterocycloalkyl, or a monocyclic or multicyclic heterocyclo-alkenyl group;
   each R⁸ is independently H, halogen, or C₁-C₄ alkyl;
   m is 0, 1, 2, 3, or 4;
   n is 0 or 1; and
   ULM is

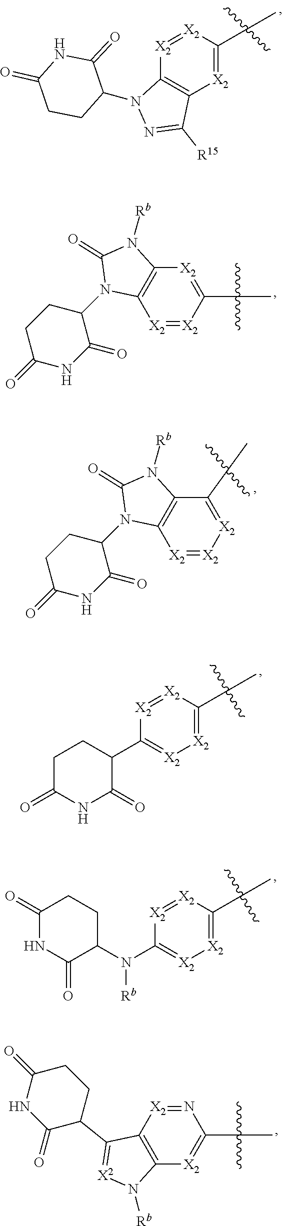

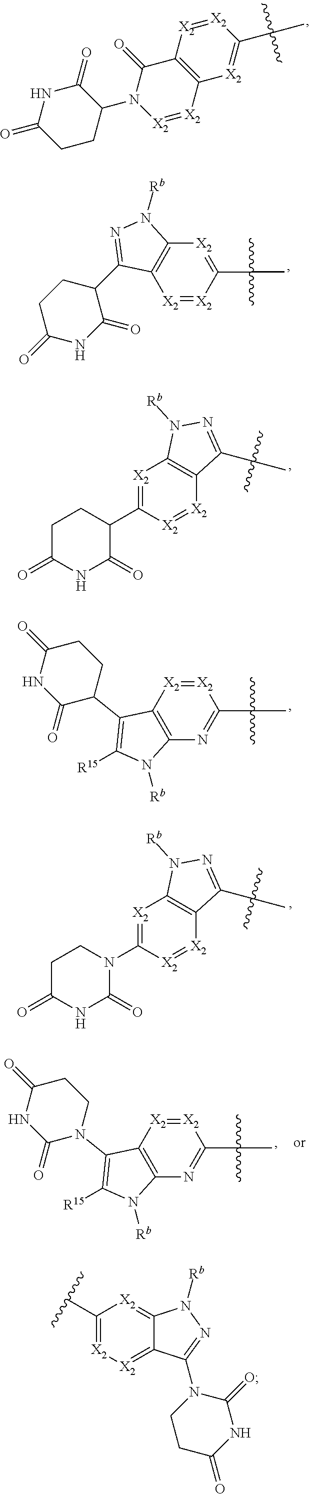

wherein
[symbol] is a point of attachment to Y;
each X₂ is independently N or CR¹⁵; and
each R¹⁵ is independently H, D, halogen, oxo, —OH, —CN, —NO₂, —C₁-C₆alkyl, —C₁-C₆ haloalkyl, —C₂-C₆alkenyl, —C₂-C₆alkynyl, C₀-C₁alk-aryl, C₀-C₁alk-heteroaryl, cycloalkyl, cycloalkenyl, heterocycloalkyl, or heterocycloalkenyl, —OR^{a}, —SR^{a}, —NR^{c}R^{d}, —NR^{a}R^{c}, —C(O)R^{b}, —OC(O)R^{a}, —C(O)OR^{a}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{d})(OR^{c}) or —S(O)₂R^{b};
   wherein any of the —C₁-C₆alkyl, C₁-C₈ alkoxide, —C₂-C₆alkenyl, —C₂-C₆alkynyl, aryl, heteroaryl, cycloalkyl, cycloalkenyl, heterocycloalkyl, heterocycloalkenyl, spirocycloalkyl, spiroheterocycloalkyl and spiroheterocycloalkenyl groups in any of R₁—R₁₅ and ring A is optionally substituted by 1-6 R^{f} groups; and
   each R^{f} is independently H, D, oxo, halogen, C₁-C₈ alkoxy, C₁-C₈ alkyl, C₁₋₆ haloalkyl, C₁₋₆ haloalkoxy, —OH, —CN, —NO₂, —C₂₋₆ alkenyl, —C₂₋₆ alkynyl, C₆₋₁₀ aryl, C₅₋₁₂ heteroaryl, C₃₋₈ cycloalkyl, C₃₋₈ cycloalkenyl, C₃₋₈ heterocycloalkyl, C₃₋₈ heterocycloalkenyl, —OR^{a}, —SR^{a}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)R^{c}R^{d}, —P(O)(OR^{b})(OR^{b}), —B(OR^{c})(OR^{d}), —S(O)₂R^{b}, —C(O)NR^{b}OR^{b}, —S(O)₂OR^{b}, —OS(O)₂OR^{b}, or —OPO(OR^{b})(OR^{b}); wherein said C₁-C₈ alkyl is optionally substituted by 1-6 groups selected from D, halogen, —OH, —CN, —OR^{a}, —SR^{a}, —NR^{a}R^{d}, or NR^{c}R^{d}.

In a second aspect of the invention, provided is a compound of the first aspect, wherein Y is a chemical moiety that links PTM and ULM.

In a third aspect of the invention, provided is a compound of the second aspect, wherein R¹ is H.

In a fourth aspect of the invention, provided is a compound of any one of the preceding aspects, wherein R² is H.

In a fifth aspect of the invention, provided is a compound of any one of the first through third aspects, wherein R² is —(C(R⁸)₂)ₙ-(5-9 membered heteroaryl) optionally substituted by halogen, C₁-C₃ alkyl, —CH₂OH, or —OH.

In a sixth aspect of the invention, provided is a compound of the fifth aspect, wherein R² is —(C(R⁸)₂)ₙ-(5-6 membered heteroaryl) optionally substituted by halogen, C₁-C₃ alkyl, —CH₂OH, or —OH.

In a seventh aspect of the invention, provided is a compound of the sixth aspect, wherein the 5-6 membered heteroaryl is a pyrazole, a pyrrole, a pyridine or a pyridazine.

In an eighth aspect of the invention, provided is a compound of the sixth aspect, wherein the 5-6 membered heteroaryl is a pyrazole.

In a ninth aspect of the invention, provided is a compound of any one of the first through third aspects, wherein R² is C₁₋₄alk-O—C₁₋₄alkyl.

In a tenth aspect of the invention, provided is a compound of the ninth aspect, wherein R² is CH₂—O—CH₃.

In an eleventh aspect of the invention, provided is a compound of any one of the preceding aspects, wherein n is 0.

In a twelfth aspect of the invention, provided is a compound of any one of the first through tenth aspects, wherein n is 1.

In a thirteenth aspect of the invention, provided is a compound of any one of the preceding aspects, wherein R³ is H.

In a fourteenth aspect of the invention, provided is a compound of any one of the first through twelfth aspects, wherein R³ is halogen.

In a fifteenth aspect of the invention, provided is a compound of any one of the first through twelfth aspects, wherein R³ is C₁-C₄ alkyl.

In a sixteenth aspect of the invention, provided is a compound of any one of the preceding aspects, wherein R⁴ is H.

In a seventeenth aspect of the invention, provided is a compound of any one of the first through fifteenth aspects, wherein R⁴ is C₁-C₄ alkyl.

In an eighteenth aspect of the invention, provided is a compound of any one of the first through fifteenth aspects, wherein R⁴ is C₁-C₄ alkoxy.

In a nineteenth aspect of the invention, provided is a compound of the eighteenth aspect, wherein R⁴ is methoxy.

In a twentieth aspect of the invention, provided is a compound of any one of the preceding aspects, wherein Ring A is C₆-C₁₀ aryl.

In a twenty first aspect of the invention, provided is a compound of the twentieth aspect, wherein Ring A is phenyl.

In a twenty second aspect of the invention, provided is a compound of any one of the preceding aspects, wherein each R⁵ is H.

In a twenty third aspect of the invention, provided is a compound of any one of the first through twenty first aspects, wherein at least one R⁵ is H.

In a twenty fourth aspect of the invention, provided is a compound of any one of the first through twenty first aspects, wherein each R₅ is C₁-C₄ alkoxy.

In a twenty fifth aspect of the invention, provided is a compound of any one of the first through twenty first aspects, wherein at least one R₅ is C₁-C₄ alkoxy.

In a twenty sixth aspect of the invention, provided is a compound of any one of the preceding aspects, wherein m is 0.

In a twenty seventh aspect of the invention, provided is a compound of any one of the first through twenty fifth aspects, wherein m is 1.

In a twenty eighth aspect of the invention, provided is a compound of any one of the first through twenty fifth aspects, wherein m is 2.

In a twenty ninth aspect of the invention, provided is a compound of any one of the first through twenty fifth aspects, wherein m is 3.

In a thirtieth aspect of the invention, provided is a compound of any one of the first through twenty fifth aspects, wherein m is 4.

In a thirty first aspect of the invention, provided is a compound of any one of the preceding aspects, wherein each R⁸ is H.

In a thirty second aspect of the invention, provided is a compound of any one of the preceding aspects, wherein Y is represented by the formula:

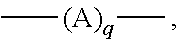

wherein:
q is an integer from 1 to 14;
   each A is independently selected from the group consisting of CR^{1a}R^{1b} O, S, SO, SO₂, NR^{1c}, SO₂NR^{1c}, SONR^{1c}, SO(═NR^{1c}), SO(═NR^{1c})NR^{1d}, CONR^{1c}, NR^{1c}CONR^{1d}, NR^{1c}C(O)O, NR^{1c}SO₂NR^{1d}, CO, CR^{1a}═CR^{1b}, C≡C, SiR^{1a}R^{1b}, P(O)R^{1a}, P(O)OR^{1a}, (CR^{1a}R^{1b})₁₋₄, —(CR^{1a}R^{1b})₁₋₄O(CR^{1a}R^{1b})₁₋₄, —(CR^{1a}R^{1b})₁₋₄S(CR^{1a}R^{1b})₁₋₄, —(CR^{1a}R^{1b})₁₋₄NR^{1c}(CR^{1a}R^{1b})₁, —NR^{1c}C(═NCN)NR^{1d}, NR^{1c}C(═NCN), NR^{1c}C(═CNO₂)NR^{1d}, 3-11 membered cycloalkyl, optionally substituted with 0-6 R^{1a} or R^{1b} groups, 3-11 membered heterocyclyl optionally substituted with 0-6 R^{1a} or R^{1b} groups, aryl optionally substituted with 0-6 R^{1a} or R^{1b} groups, or heteroaryl optionally substituted with 0-6 R^{1a} or R^{1b} groups,
   wherein R^{1a}, R^{1b}, R^{1c}, R^{1d} and R^{1c} are each independently, —H, -halo, —C₁-C₈alkyl, —O—C₁-C₈alkyl, —C₁-C₆haloalkyl, —S—C₁-C₈alkyl, —NHC₁-C₈alkyl, —N(C₁-C₈alkyl)₂, 3-11 membered cycloalkyl, aryl, heteroaryl, 3-11 membered heterocyclyl, —O-(3-11 membered cycloalkyl), —S-(3-11 membered cycloalkyl), NH-(3-11 membered cycloalkyl), N(3-11 membered cycloalkyl)₂, N-(3-11 membered cycloalkyl)(C₁-C₈alkyl), —OH, —NH₂, —SO₂C₁-C₈alkyl, —SO₂-aryl, —SO₂-heteroaryl, SO(NH)C₁-C₈alkyl, P(O)(OC₁-C₈alkyl)(C₁-C₈alkyl), —P(O)(OC₁-C₈alkyl)₂, —C≡C—C₁-C₈alkyl, —C≡CH, —CH═CH(C₁-C₈alkyl), —C(C₁-C₈alkyl)═CH(C₁-C₈alkyl), —C(C₁-C₈alkyl)═C(C₁-C₈alkyl)₂, —Si(OH)₃, —Si(C₁-C₈alkyl)₃, —Si(OH)(C₁-C₈alkyl)₂, —C(O)C₁-C₈alkyl, —C(O)OC₁-C₈alkyl, —CO₂H, —CN, —CF₃, —CHF₂, —CH₂F, —NO₂, —SF₅, —SO₂NHC₁-C₈alkyl, —SO₂N(C₁-C₈alkyl)₂, —SO(NH)NHC₁-C₈alkyl, —SO(NH)N(C₁-C₈alkyl)₂, —SONHC₁-C₈alkyl, —SON(C₁-C₈alkyl)₂, —CONHC₁-C₈alkyl, —CON(C₁-C₈alkyl)₂, —N(C₁-C₈alkyl)CONH(C₁-C₈alkyl), —N(C₁-C₈alkyl)CON(C₁-C₈alkyl)₂, —NHCONH(C₁-C₈alkyl), —NHCON(C₁-C₈alkyl)₂, —NHCONH₂, —N(C₁-C₈alkyl)SO₂NH(C₁-C₈alkyl), —N(C₁-C₈alkyl)SO₂N(C₁-C₈alkyl)₂, —NHSO₂NH(C₁-C₈alkyl), —NHSO₂N(C₁-C₈alkyl)₂, or —NHSO₂NH₂; and where R^{1a} or R^{1b}, each independently may be optionally linked to other groups to form cycloalkyl and/or heterocyclyl moiety, optionally substituted with 0-4 R^{1c} groups.

In a thirty third aspect of the invention, provided is a compound of the thirty second aspect, wherein q is an integer from 1 to 5.

In a thirty fourth aspect of the invention, provided is a compound of any one of the first through thirty first aspects, wherein Y is

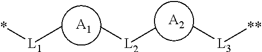

wherein
** is a point of attachment to PTM;
L₁, L₂, and L₃ are each independently a bond, CR^{1a}R^{1b}, O, S, SO, SO₂, NR^{1c}, SO₂NR^{1c}, SONR^{1c}, SO(═NR^{1c}), SO(═NR^{1c})NR^{1d}, CONR^{1c}, NR^{1c}CONR^{1d}, NR^{1c}C(O)O, NR^{1c}SO₂NR^{1d}, CO, CR^{1a}═CR^{1b}, C≡C, SiR^{1a}R^{1b}, P(O)R^{1a}, P(O)OR^{1a} (CR^{1a}R^{1b})₁₋₄, —(CR^{1a}R^{1b})₁₋₄O(CR^{1a}R^{1b})₁₋₄, —(CR^{1a}R^{1b})₁₋₄S(CR^{1a}R^{1b})₁₋₄, —(CR^{1a}R^{1b})₁₋₄NR^{1c}(CR^{1a}R^{1b})₁₋₄, NR^{1c}C(═NCN)NR^{1d}, NR^{1c}C(═NCN), NR^{1c}C(═CNO₂)NR^{1d};
ring A₁ and ring A₂ are each independently 3-12 membered cycloalkyl, optionally substituted with 0-6 R^{1a} or R^{1b} groups, 3-12 membered heterocyclyl optionally substituted with 0-6 R^{1a} or R^{1b} groups, aryl optionally substituted with 0-6 R^{1a} or R^{1b} groups, or heteroaryl optionally substituted with 0-6 R^{1a} or R^{1b} groups, wherein R^{1a}, R^{1b}, R^{1c}, R^{1d} and R^{1e} are each independently, —H, -halo, —C₁-C₈alkyl, —O—C₁-C₈alkyl, —C₁-C₆haloalkyl, —S—C₁-C₈alkyl, —NHC₁-C₈alkyl, —N(C₁-C₈alkyl)₂, 3-11 membered cycloalkyl, aryl, heteroaryl, 3-11 membered heterocyclyl, —O-(3-11 membered cycloalkyl), —S-(3-11 membered cycloalkyl), NH-(3-11 membered cycloalkyl), N(3-11 membered cycloalkyl)₂, N-(3-11 membered cycloalkyl)(C₁-C₈alkyl), —OH, —NH₂, —SH, —SO₂C₁-C₈alkyl, —SO₂-aryl, —SO₂-heteroaryl, SO(NH)C₁-C₈alkyl, P(O)(OC₁-C₈alkyl)(C₁-C₈alkyl), —P(O)(OC₁-C₈alkyl)₂, —C≡C—C₁-C₈alkyl, —C≡CH, —CH═CH(C₁-C₈alkyl), —C(C₁-C₈alkyl)═CH(C₁-C₈alkyl), —C(C₁-C₈alkyl)═C(C₁-C₈alkyl)₂, —Si(OH)₃, —Si(C₁-C₈alkyl)₃, —Si(OH)(C₁-C₈alkyl)₂, —C(O)C₁-C₈alkyl, —C(O)OC₁-C₈alkyl, —CO₂H, —CN, —CF₃, —CHF₂, —CH₂F, —NO₂, —SF₅, —SO₂NHC₁-C₈alkyl, —SO₂N(C₁-C₈alkyl)₂, —SO(NH)NHC₁-C₈alkyl, —SO(NH)N(C₁-C₈alkyl)₂, —SONHC₁-C₈alkyl, —SON(C₁-C₈alkyl)₂, —CONHC₁-C₈alkyl, —CON(C₁-C₈alkyl)₂, —N(C₁-C₈alkyl)CONH(C₁-C₈alkyl), —N(C₁-C₈alkyl)CON(C₁-C₈alkyl)₂, —NHCONH(C₁-C₈alkyl), —NHCON(C₁-C₈alkyl)₂, —NHCONH₂, —N(C₁-C₈alkyl)SO₂NH(C₁-C₈alkyl), —N(C₁-C₈alkyl)SO₂N(C₁-C₈alkyl)₂, —NHSO₂NH(C₁-C₈alkyl), —NHSO₂N(C₁-C₈alkyl)₂, or —NHSO₂NH₂; and where R^{1a} or R^{1b}, each independently may be optionally linked to other groups to form cycloalkyl and/or heterocyclyl moiety, optionally substituted with 0-4 R^{1c} groups.

In a thirty fifth aspect of the invention, provided is a compound of any one of the first through thirty first aspects, wherein Y is

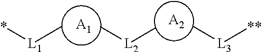

wherein
** is a point of attachment to PTM;
L₁ is a bond, (C(R¹⁰)₂)ₚ, or CO;
L₂ is a bond, (C(R¹⁰)₂)ₚ, or CO;
L₃ is a bond, (C(R¹⁰)₂)ₚ, or CO;
p is 1, 2, 3 or 4;
each Rio is independently H or C₁-C₄ alkyl;
ring A₁ is a 3-7 membered cycloalkyl group, a 4-10-membered heterocycloalkyl group, an aryl group, or a heteroaryl group; and
ring A₂ is a 3-7 membered cycloalkyl group, a 4-12-membered heterocycloalkyl group, an aryl group, or a heteroaryl group.

In a thirty sixth aspect of the invention, provided is a compound of thirty fifth aspect, wherein L₃ is a bond.

In a thirty seventh aspect of the invention, provided is a compound of thirty fifth aspect or the thirty sixth aspect, wherein L₁ is a bond.

In a thirty eighth aspect of the invention, provided is a compound of any one of the thirty fifth through thirty seventh aspects, wherein L₂ is (C(R¹⁰)₂)ₚ.

In a thirty ninth aspect of the invention, provided is a compound of any one of the thirty fifth through thirty eighth aspects, wherein ring A₁ is a 4-10-membered heterocycloalkyl group.

In a fortieth aspect of the invention, provided is a compound of thirty ninth aspect, wherein ring A₁ is a piperazine group, a morpholine group, a piperidine group, a pyrrolidine group, an azetidine group or an azabicyclo-alkyl group.

In a forty first aspect of the invention, provided is a compound of thirty ninth aspect, wherein ring A₁ is a piperidine group or pyrrolidine group.

In a forty second aspect of the invention, provided is a compound of any one of the thirty fifth through forty first aspects, wherein ring A₂ is a 4-12-membered heterocycloalkyl group.

In a forty third aspect of the invention, provided is a compound of the forty second aspect, wherein ring A₂ is a piperazine group, a piperidine group, a pyrrolidine group, an azaspiroalkyl group, a diazaspiroalkyl group, or an azabicycloalkyl group.

In a forty fourth aspect of the invention, provided is a compound of the forty second aspect, wherein ring A₂ is a piperazine group, an azaspiroalkyl group, or a diazaspiroalkyl group.

In a forty fifth aspect of the invention, provided is a compound of the forty second aspect, wherein ring A₂ is aryl optionally substituted with 0-6 R^{1a} or R^{1b} groups.

In a forty sixth aspect of the invention, provided is a compound of the thirty fifth aspect, wherein Y is

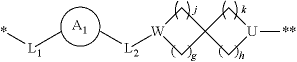

wherein
* is a point of attachment to ULM;
** is a point of attachment to PTM;
W is CR₁₁ or N;
U is CR₁₁ or N;
R₁₁ is H, fluoro, C₁₋₆alkyl, or C₁₋₆alkoxide; and
each g, h, j and k is independently 0, 1 or 2.

In a forty seventh aspect of the invention, provided is a compound of the forty sixth aspect, wherein j and g are each 2.

In a forty eighth aspect of the invention, provided is a compound of the forty sixth aspect or the forty seventh aspect, wherein k and h are each 1.

In a forty ninth aspect of the invention, provided is a compound of the forty sixth aspect, wherein j and g are each 1.

In a fiftieth aspect of the invention, provided is a compound of the forty sixth aspect or the forty seventh aspect, wherein k and h are each 2.

In a fifty first aspect of the invention, provided is a compound of any one of the forty sixth through fiftieth aspects, wherein W and U are N.

In a fifty second aspect of the invention, provided is a compound of any one of the forty sixth through fiftieth aspects, wherein W is CR₁₁ and U are N.

In a fifty third aspect of the invention, provided is a compound of any one of the forty sixth through fiftieth aspects, wherein W is N and U is CR₁₁.

In a fifty fourth aspect of the invention, provided is a compound of any one of the preceding aspects, wherein Ring A is selected from

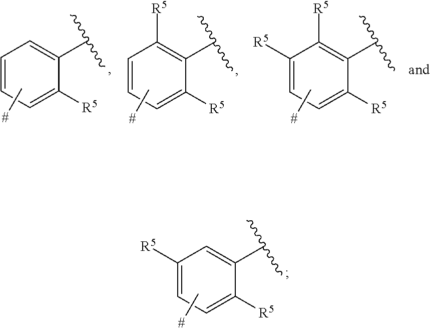

wherein
[symbol] is a point of attachment to the sulfonamide group and # is a point of attachment to Y.

In a fifty fifth aspect of the invention, provided is a compound of the fifty fourth aspect, wherein Ring A is selected from

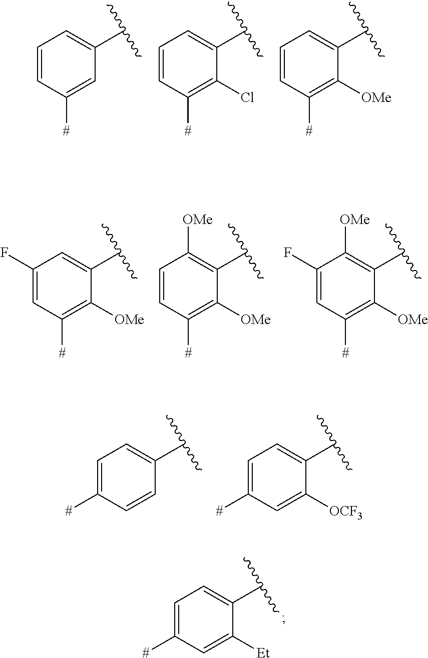

wherein
[symbol] is a point of attachment to the sulfonamide group and # is a point of attachment to Y.

In a fifty sixth aspect of the invention, provided is a compound of the first aspect, wherein Y-PTM is a compound of formula IA-1:

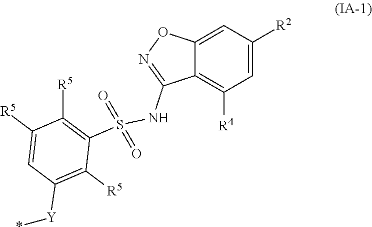

or a pharmaceutically acceptable salt thereof.

In a fifty seventh aspect of the invention, provided is a compound of the first aspect, wherein Y-PTM is a compound of formula IA-2:

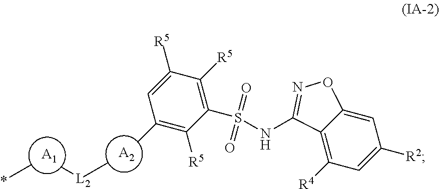

or a pharmaceutically acceptable salt thereof, wherein
* is a point of connection to ULM;
L₂ is a bond or (C(R¹⁰)₂)ₚ;
p is 1, 2, 3 or 4;
each R¹⁰ is independently H or C₁-C₄ alkyl;
ring A₁ is a 3-7 membered cycloalkyl group, a 4-10-membered heterocycloalkyl group, an aryl group, or a heteroaryl group; and
ring A₂ is a 3-7 membered cycloalkyl group, a 4-12-membered heterocycloalkyl group, an aryl group, or a heteroaryl group.

In a fifty eighth aspect of the invention, provided is a compound of the first aspect, wherein Y-PTM is a compound of formula IA-3:

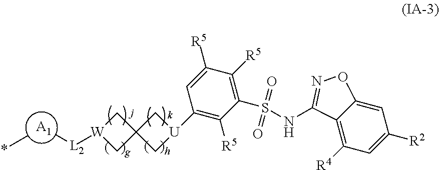

or a pharmaceutically acceptable salt thereof, wherein
* is a point of connection to ULM;
L₂ is a bond or (C(R¹⁰)₂)ₚ;
p is 1, 2, 3 or 4;
each R¹⁰ is independently H or C₁-C₄ alkyl;
ring A₁ is a 3-7 membered cycloalkyl group, a 4-10-membered heterocycloalkyl group, an aryl group, or a heteroaryl group;
W is CR₁₁ or N;
U is CR₁₁ or N;
R₁₁ is H, F, C₁₋₆alkyl, or C₁₋₆alkoxide; and
each g, h, j and k is independently 0, 1, 2 or 3.

In a fifty eighth aspect of the invention, provided is a compound of the fifty eighth aspect, wherein j and g are each 2.

In a sixtieth aspect of the invention, provided is a compound of the fifty eighth aspect or the fifty ninth aspect, wherein k and h are each 1.

In a sixth first aspect of the invention, provided is a compound of the fifty eighth aspect, wherein j and g are each 1.

In a sixty second aspect of the invention, provided is a compound of the fifty eighth aspect or the fifty ninth aspect, wherein k and h are each 2.

In a sixty third aspect of the invention, provided is a compound of any one of the fifty eighth through sixty second aspects, wherein W and U are N.

In a sixty fourth aspect of the invention, provided is a compound of any one of the fifty eighth through sixty second aspects, wherein W is CR₁₁ and U are N.

In a sixty fifth aspect of the invention, provided is a compound of any one of the fifty eighth through sixty second aspects, wherein W is N and U is CR₁₁.

In a sixty sixth aspect of the invention, provided is a compound of any one of the preceding aspects, wherein Y-PTM is a compound of formula IA-4 or formula IA-5:

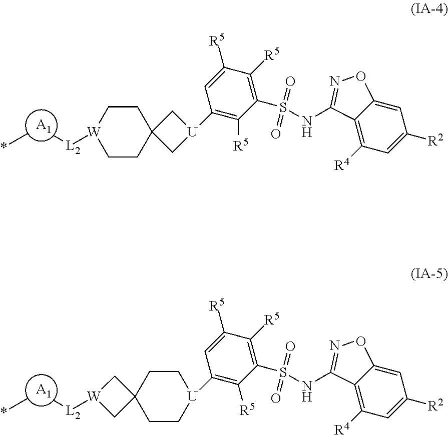

or a pharmaceutically acceptable salt thereof, wherein
* is a point of connection to ULM;
L₂ is a bond or (C(R¹⁰)₂)ₚ;
p is 1, 2, 3 or 4;
each R¹⁰ is independently H or C₁-C₄ alkyl;
ring A₁ is a 3-7 membered cycloalkyl group, a 4-10-membered heterocycloalkyl group, an aryl group, or a heteroaryl group;
W is CR₁₁ or N;
U is CR₁₁ or N; and
R₁₁ is H, F, C₁₋₆alkyl, or C₁₋₆alkoxide.

In a sixty seventh aspect of the invention, provided is a compound of the sixty sixth aspect, wherein Y-PTM is a compound of formula IA-6 or formula IA-7:

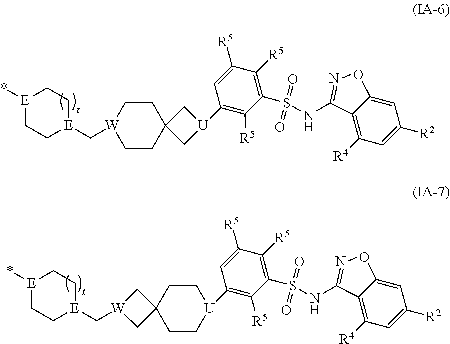

or a pharmaceutically acceptable salt thereof, wherein
* is a point of connection to ULM;
W is CR₁₁ or N;
U is CR₁₁ or N; and
R¹¹ is H, F, C₁₋₆alkyl, or C₁₋₆alkoxide;
each E is independently CR₁₁ or N; and
t is 0, 1, 2 or 3.

In a sixty eighth aspect of the invention, provided is a compound of any one of the fifty sixth through sixty seventh aspects, wherein each R⁵ is independently H, —CN, halogen, C₁-C₆ alkyl, C₁-C₆ fluoroalkyl, O—C₁-C₆ fluoroalkyl, C₃-C₆ cycloalkyl or C₁-C₆ alkoxy.

In a sixty ninth aspect of the invention, provided is a compound of any one of the preceding aspects, wherein each X₂ is CR^{b}.

In a seventieth aspect of the invention, provided is a compound of any one of the preceding aspects, wherein each X₂ is CH.

In a seventy first aspect of the invention, provided is a compound of any one of the preceding aspects, wherein ULM is

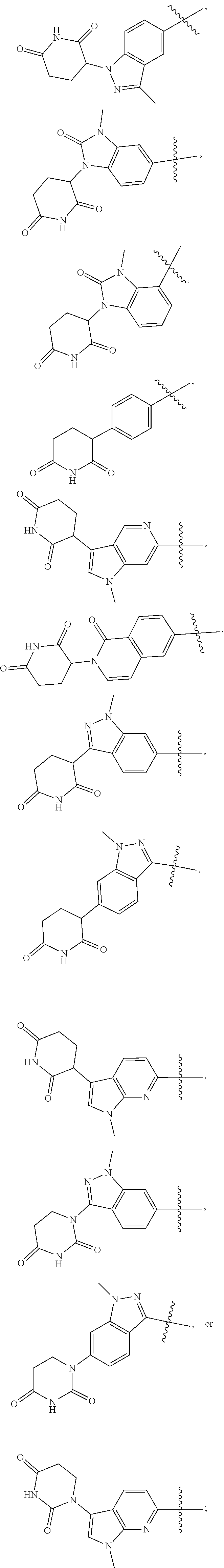

wherein [symbol] is a point of attachment to Y.

In a seventy second aspect of the invention, provided is a compound of the first aspect, that is:
N-(6-((1H-Pyrazol-1-yl)methyl)-4-methoxybenzo[d]isoxazol-3-yl)-3-(7-(((3S)-1-(1-(2,6-dioxopiperidin-3-yl)-3-methyl-1H-indazol-5-yl)pyrrolidin-3-yl)methyl)-2,7-diazaspiro[3.5]nonan-2-yl)benzenesulfonamide;
N-(6-((1H-Pyrazol-1-yl)methyl)-4-methoxybenzo[d]isoxazol-3-yl)-3-(7-((1-(1-(2,6-dioxopiperidin-3-yl)-3-methyl-2-oxo-2,3-dihydro-1H-benzo[d]imidazol-5-yl)piperidin-4-yl)methyl)-2,7-diazaspiro[3.5]nonan-2-yl)benzenesulfonamide;
N-(6-((1H-Pyrazol-1-yl)methyl)-4-methoxybenzo[d]isoxazol-3-yl)-3-(7-((1-(4-(2,6-dioxopiperidin-3-yl)phenyl)pyrrolidin-3-yl)methyl)-2,7-diazaspiro[3.5]nonan-2-yl)benzenesulfonamide;
N-(6-((1H-Pyrazol-1-yl)methyl)-4-methoxybenzo[d]isoxazol-3-yl)-3-(7-((1-(3-(2,6-dioxopiperidin-3-yl)-1-methyl-1H-pyrrolo[3,2-c]pyridin-6-yl)piperidin-4-yl)methyl)-2,7-diazaspiro[3.5]nonan-2-yl)benzenesulfonamide;
N-(6-((1H-Pyrazol-1-yl)methyl)-4-methoxybenzo[d]isoxazol-3-yl)-3-(7-((1-(2-(2,6-dioxopiperidin-3-yl)-1-oxo-1,2-dihydroisoquinolin-6-yl)piperidin-4-yl)methyl)-2,7-diazaspiro[3.5]nonan-2-yl)-2-methoxybenzenesulfonamide;
N-(6-((1H-Pyrazol-1-yl)methyl)-4-methoxybenzo[d]isoxazol-3-yl)-3-(7-((1-(1-(2,6-dioxopiperidin-3-yl)-3-methyl-1H-indazol-5-yl)piperidin-4-yl)methyl)-2,7-diazaspiro[3.5]nonan-2-yl)-2-methoxybenzenesulfonamide;
N-(6-((1H-Pyrazol-1-yl)methyl)-4-methoxybenzo[d]isoxazol-3-yl)-3-(7-((1-(3-(2,6-dioxopiperidin-3-yl)-1-methyl-1H-indazol-6-yl)piperidin-4-yl)methyl)-2,7-diazaspiro[3.5]nonan-2-yl)-2-methoxybenzenesulfonamide;
N-(6-((1H-Pyrazol-1-yl)methyl)-4-methoxybenzo[d]isoxazol-3-yl)-3-(7-((1-(6-(2,6-dioxopiperidin-3-yl)-1-methyl-1H-indazol-3-yl)piperidin-4-yl)methyl)-2,7-diazaspiro[3.5]nonan-2-yl)-2-methoxybenzenesulfonamide;
N-(6-((1H-Pyrazol-1-yl)methyl)-4-methoxybenzo[d]isoxazol-3-yl)-3-(7-((1-(3-(2,6-dioxopiperidin-3-yl)-1-methyl-1H-pyrrolo[2,3-b]pyridin-6-yl)piperidin-4-yl)methyl)-2,7-diazaspiro[3.5]nonan-2-yl)-2-methoxybenzenesulfonamide;
N-(6-((1H-Pyrazol-1-yl)methyl)-4-methoxybenzo[d]isoxazol-3-yl)-3-(7-((1-(3-(2,4-dioxotetrahydropyrimidin-1(2H)-yl)-1-methyl-1H-indazol-6-yl)piperidin-4-yl)methyl)-2,7-diazaspiro[3.5]nonan-2-yl)-2,4-dimethoxybenzenesulfonamide;
N-(6-((1H-Pyrazol-1-yl)methyl)-4-methoxybenzo[d]isoxazol-3-yl)-3-(7-((1-(3-(2,6-dioxopiperidin-3-yl)-1-methyl-1H-indazol-6-yl)piperidin-4-yl)methyl)-2,7-diazaspiro[3.5]nonan-2-yl)-2,6-dimethoxybenzenesulfonamide;
N-(6-((1H-Pyrazol-1-yl)methyl)-4-methoxybenzo[d]isoxazol-3-yl)-4-(7-((1-(1-(2,6-dioxopiperidin-3-yl)-3-methyl-1H-indazol-5-yl)piperidin-4-yl)methyl)-2,7-diazaspiro[3.5]nonan-2-yl)benzenesulfonamide;
N-(6-((1H-Pyrazol-1-yl)methyl)-4-methoxybenzo[d]isoxazol-3-yl)-4-(7-((1-(2-(2,6-dioxopiperidin-3-yl)-1-oxo-1,2-dihydroisoquinolin-6-yl)piperidin-4-yl)methyl)-2,7-diazaspiro[3.5]nonan-2-yl)benzenesulfonamide;
N-(6-((1H-Pyrazol-1-yl)methyl)-4-methoxybenzo[d]isoxazol-3-yl)-4-(7-((1-(1-(2,6-dioxopiperidin-3-yl)-3-methyl-1H-indazol-5-yl)piperidin-4-yl)methyl)-2,7-diazaspiro[3.5]nonan-2-yl)-2-methoxybenzenesulfonamide;
N-(6-((1H-Pyrazol-1-yl)methyl)-4-methoxybenzo[d]isoxazol-3-yl)-4-(7-((1-(1-(2,6-dioxopiperidin-3-yl)-3-methyl-2-oxo-2,3-dihydro-1H-benzo[d]imidazol-5-yl)piperidin-4-yl)methyl)-2,7-diazaspiro[3.5]nonan-2-yl)-2-methoxybenzenesulfonamide;
N-(6-((1H-Pyrazol-1-yl)methyl)-4-methoxybenzo[d]isoxazol-3-yl)-4-(4-((1-(1-(2,6-dioxopiperidin-3-yl)-3-methyl-2-oxo-2,3-dihydro-1H-benzo[d]imidazol-5-yl)piperidin-4-yl)methyl)-3,3-dimethylpiperazin-1-yl)benzenesulfonamide;
N-(6-((1H-Pyrazol-1-yl)methyl)-4-methoxybenzo[d]isoxazol-3-yl)-4-((3aS,6aS)-5-((1-(1-(2,6-dioxopiperidin-3-yl)-3-methyl-2-oxo-2,3-dihydro-1H-benzo[d]imidazol-5-yl)piperidin-4-yl)methyl)hexahydropyrrolo[3,4-c]pyrrol-2(1H)-yl)benzenesulfonamide;
N-(6-((1H-Pyrazol-1-yl)methyl)-4-methoxybenzo[d]isoxazol-3-yl)-4-((3aR,6aS)-5-((1-(1-(2,6-dioxopiperidin-3-yl)-3-methyl-2-oxo-2,3-dihydro-1H-benzo[d]imidazol-5-yl)piperidin-4-yl)methyl)hexahydropyrrolo[3,4-c]pyrrol-2(1H)-yl)-2-methoxybenzenesulfonamide;
N-(6-((1H-Pyrazol-1-yl)methyl)-4-methoxybenzo[d]isoxazol-3-yl)-3-(9-((1-(4-(2,6-dioxopiperidin-3-yl)phenyl)piperidin-4-yl)methyl)-3,9-diazaspiro[5.5]undecan-3-yl)benzenesulfonamide;
N-(6-((1H-Pyrazol-1-yl)methyl)-4-methoxybenzo[d]isoxazol-3-yl)-3-(9-((1-(3-(2,6-dioxopiperidin-3-yl)-1-methyl-1H-indazol-6-yl)piperidin-4-yl)methyl)-3,9-diazaspiro[5.5]undecan-3-yl)benzenesulfonamide;
N-(6-((1H-Pyrazol-1-yl)methyl)-4-methoxybenzo[d]isoxazol-3-yl)-3-(7-((1-(3-(2,6-dioxopiperidin-3-yl)-1-methyl-1H-indazol-6-yl)piperidin-4-yl)methyl)-2,7-diazaspiro[3.5]nonan-2-yl)-2-(2,2,2-trifluoroethoxy)benzenesulfonamide;
N-(6-((1H-Pyrazol-1-yl)methyl)-4-methoxybenzo[d]isoxazol-3-yl)-2-chloro-3-(7-((1-(3-(2,6-dioxopiperidin-3-yl)-1-methyl-1H-indazol-6-yl)piperidin-4-yl)methyl)-2,7-diazaspiro[3.5]nonan-2-yl)benzenesulfonamide;
N-(6-((1H-Pyrazol-1-yl)methyl)-4-methoxybenzo[d]isoxazol-3-yl)-3-(7-((1-(3-(2,6-dioxopiperidin-3-yl)-1-methyl-1H-indazol-6-yl)piperidin-4-yl)methyl)-2,7-diazaspiro[3.5]nonan-2-yl)-5-fluoro-2-methoxybenzenesulfonamide;
N-(6-((1H-Pyrazol-1-yl)methyl)-4-methoxybenzo[d]isoxazol-3-yl)-5-chloro-3-(7-((1-(3-(2,6-dioxopiperidin-3-yl)-1-methyl-1H-indazol-6-yl)piperidin-4-yl)methyl)-2,7-diazaspiro[3.5]nonan-2-yl)-2-methoxybenzenesulfonamide;
N-(6-((1H-Pyrazol-1-yl)methyl)-4-methoxybenzo[d]isoxazol-3-yl)-3-(7-((1-(3-(2,6-dioxopiperidin-3-yl)-1-methyl-1H-indazol-6-yl)piperidin-4-yl)methyl)-2,7-diazaspiro[3.5]nonan-2-yl)-2-methoxy-5-methylbenzenesulfonamide;
N-(6-((1H-pyrazol-1-yl)methyl)-4-methoxybenzo[d]isoxazol-3-yl)-3-(7-((1-(3-(2,6-dioxopiperidin-3-yl)-1-methyl-1H-indazol-6-yl)piperidin-4-yl)methyl)-2,7-diazaspiro[3.5]nonan-2-yl)-2-(trifluoromethyl)benzenesulfonamide;
N-(6-((1H-pyrazol-1-yl)methyl)-4-methoxybenzo[d]isoxazol-3-yl)-3-(7-((1-(3-(2,6-dioxopiperidin-3-yl)-1-methyl-1H-indazol-6-yl)piperidin-4-yl)methyl)-2,7-diazaspiro[3.5]nonan-2-yl)-2-fluorobenzenesulfonamide;
N-(6-((1H-Pyrazol-1-yl)methyl)-4-methoxybenzo[d]isoxazol-3-yl)-3-(7-((1-(3-(2,6-dioxopiperidin-3-yl)-1-methyl-1H-indazol-6-yl)piperidin-4-yl)methyl)-2,7-diazaspiro[3.5]nonan-2-yl)-5-fluoro-2,6-dimethoxybenzenesulfonamide;
N-(6-((1H-Pyrazol-1-yl)methyl)-4-methoxybenzo[d]isoxazol-3-yl)-5-(7-((1-(4-(2,6-dioxopiperidin-3-yl)phenyl)piperidin-4-yl)methyl)-2,7-diazaspiro[3.5]nonan-2-yl)-2-methylbenzenesulfonamide;
N-(6-((1H-Pyrazol-1-yl)methyl)-4-methoxybenzo[d]isoxazol-3-yl)-3-(7-((1-(4-(2,6-dioxopiperidin-3-yl)phenyl)piperidin-4-yl)methyl)-2,7-diazaspiro[3.5]nonan-2-yl)-2-methylbenzenesulfonamide;
N-(6-((1H-Pyrazol-1-yl)methyl)-4-methoxybenzo[d]isoxazol-3-yl)-4-(7-((1-(3-(2,6-dioxopiperidin-3-yl)-1-methyl-1H-indazol-6-yl)piperidin-4-yl)methyl)-2,7-diazaspiro[3.5]nonan-2-yl)-2-methylbenzenesulfonamide;
N-(6-((1H-Pyrazol-1-yl)methyl)-4-methoxybenzo[d]isoxazol-3-yl)-4-(7-((1-(3-(2,6-dioxopiperidin-3-yl)-1-methyl-1H-indazol-6-yl)piperidin-4-yl)methyl)-2,7-diazaspiro[3.5]nonan-2-yl)-2-ethylbenzenesulfonamide;
N-(6-((1H-Pyrazol-1-yl)methyl)-4-methoxybenzo[d]isoxazol-3-yl)-4-(7-((1-(3-(2,6-dioxopiperidin-3-yl)-1-methyl-1H-indazol-6-yl)piperidin-4-yl)methyl)-2,7-diazaspiro[3.5]nonan-2-yl)-2-(trifluoromethoxy)benzenesulfonamide;
N-(6-((1H-Pyrazol-1-yl)methyl)-4-methoxybenzo[d]isoxazol-3-yl)-3-(7-((1-(3-(2,6-dioxopiperidin-3-yl)-1-methyl-1H-indazol-6-yl)piperidin-4-yl)methyl)-7-azaspiro[3.5]nonan-2-yl)benzenesulfonamide;
N-(6-((1H-Pyrazol-1-yl)methyl)-4-methoxybenzo[d]isoxazol-3-yl)-3-(2-((4-(3-(2,4-dioxotetrahydropyrimidin-1(2H)-yl)-1-methyl-1H-indazol-6-yl)piperidin-1-yl)methyl)-7-azaspiro[3.5]nonan-7-yl)benzenesulfonamide;
N-(6-((1H-Pyrazol-1-yl)methyl)-4-methoxybenzo[d]isoxazol-3-yl)-3-(2-((4-(3-(2,6-dioxopiperidin-3-yl)-1-methyl-1H-indazol-6-yl)piperidin-1-yl)methyl)-7-azaspiro[3.5]nonan-7-yl)benzenesulfonamide;
N-(6-((1H-Pyrazol-1-yl)methyl)-4-methoxybenzo[d]isoxazol-3-yl)-3-(2-((4-((1-(2,6-dioxopiperidin-3-yl)-3-methyl-2-oxo-2,3-dihydro-1H-benzo[d]imidazol-4-yl)ethynyl)piperidin-1-yl)methyl)-7-azaspiro[3.5]nonan-7-yl)benzenesulfonamide;
or a pharmaceutically acceptable salt thereof.

In a seventy third aspect of the invention, provided is a compound of the first aspect, that is:
3-(7-((1-(3-(2,6-Dioxopiperidin-3-yl)-1-methyl-1H-indazol-6-yl)piperidin-4-yl)methyl)-2,7-diazaspiro[3.5]nonan-2-yl)-5-fluoro-2,6-dimethoxy-N-(4-methoxy-6-(methoxymethyl)-benzo[d]isoxazol-3-yl)benzenesulfonamide;
3-(7-((1-(3-(2,4-Dioxotetrahydropyrimidin-1(2H)-yl)-1-methyl-1H-indazol-6-yl)-piperidin-4-yl)methyl)-2,7-diazaspiro[3.5]nonan-2-yl)-5-fluoro-2,6-dimethoxy-N-(4-methoxy-6-(methoxymethyl)benzo[d]isoxazol-3-yl)benzenesulfonamide;
3-(7-((1-(3-(2,6-Dioxopiperidin-3-yl)-1-methyl-1H-pyrrolo[2,3-b]pyridin-6-yl)piperidin-4-yl)methyl)-2,7-diazaspiro[3.5]nonan-2-yl)-5-fluoro-2,6-dimethoxy-N-(4-methoxy-6-(methoxy-methyl)benzo[d]isoxazol-3-yl)benzenesulfonamide;
3-(2-((1-(3-(2,6-Dioxopiperidin-3-yl)-1-methyl-1H-indazol-6-yl)piperidin-4-yl)methyl)-1,2,3,4-tetrahydroisoquinolin-6-yl)-5-fluoro-2,6-dimethoxy-N-(4-methoxy-6-(methoxymethyl)-benzo[d]isoxazol-3-yl)benzenesulfonamide;
3-(2-((1-(3-(2,4-Dioxotetrahydropyrimidin-1(2H)-yl)-1-methyl-1H-indazol-6-yl)-piperidin-4-yl)methyl)-1,2,3,4-tetrahydroisoquinolin-6-yl)-5-fluoro-2,6-dimethoxy-N-(4-methoxy-6-(methoxymethyl)benzo[d]isoxazol-3-yl)benzenesulfonamide;
4′-((4-(3-(2,6-Dioxopiperidin-3-yl)-1-methyl-1H-indazol-6-yl)piperidin-1-yl)methyl)-5-fluoro-2,4-dimethoxy-N-(4-methoxy-6-(methoxymethyl)benzo[d]isoxazol-3-yl)-3′-methyl-[1,1′-biphenyl]-3-sulfonamide;
4′-((4-(3-(2,4-Dioxotetrahydropyrimidin-1(2H)-yl)-1-methyl-1H-indazol-6-yl)piperidin-1-yl)methyl)-5-fluoro-2,4-dimethoxy-N-(4-methoxy-6-(methoxymethyl)benzo-[d]isoxazol-3-yl)-3′-methyl-[1,1′-biphenyl]-3-sulfonamide;
N-(6-((1H-Pyrazol-1-yl)methyl)-4-methoxybenzo[d]isoxazol-3-yl)-3-(7-((1-(3-(2,4-dioxotetrahydropyrimidin-1(2H)-yl)-1-methyl-1H-indazol-6-yl)piperidin-4-yl)methyl)-2,7-diazaspiro[3.5]nonan-2-yl)-5-fluoro-2,6-dimethoxybenzenesulfonamide;
rac-3-((cis)-3-((1-(3-(2,4-Dioxotetrahydropyrimidin-1(2H)-yl)-1-methyl-1H-indazol-6-yl)piperidin-4-yl)methyl)-3,7-diazabicyclo[4.2.0]octan-7-yl)-5-fluoro-2,6-dimethoxy-N-(4-methoxy-6-(methoxymethyl)benzo[d]isoxazol-3-yl)benzenesulfonamide;
3-((1-(3-(2,6-Dioxopiperidin-3-yl)-1-methyl-1H-pyrrolo[2,3-b]pyridin-6-yl)piperidin-4-yl)methyl)-3,7-diazabicyclo[4.2.0]octan-7-yl)-5-fluoro-2,6-dimethoxy-N-(4-methoxy-6-(methoxymethyl)benzo[d]isoxazol-3-yl)benzenesulfonamide;
rac-3-((cis)-3-((1-(3-(2,4-Dioxotetrahydropyrimidin-1(2H)-yl)-1-methyl-1H-indazol-6-yl)piperidin-4-yl)methyl)-3,7-diazabicyclo[4.2.0]octan-7-yl)-2-methoxy-N-(4-methoxy-6-(methoxymethyl)benzo[d]isoxazol-3-yl)-5-(trifluoromethyl)benzenesulfonamide;
3-((1-(3-(2,6-Dioxopiperidin-3-yl)-1-methyl-1H-pyrrolo[2,3-b]pyridin-6-yl)piperidin-4-yl)methyl)-3,7-diazabicyclo[4.2.0]octan-7-yl)-2-methoxy-N-(4-methoxy-6-(methoxymethyl)-benzo[d]isoxazol-3-yl)-5-(trifluoromethyl)benzenesulfonamide;
N-(6-((1H-pyrazol-1-yl)methyl)-4-methoxybenzo[d]isoxazol-3-yl)-3-(6-((1-(3-(2,4-dioxotetrahydropyrimidin-1(2H)-yl)-1-methyl-1H-indazol-6-yl)piperidin-4-yl)methyl)-5,6,7,8-tetrahydro-1,6-naphthyridin-2-yl)-5-fluoro-2,6-dimethoxybenzene-sulfonamide;
N-(6-((1H-pyrazol-1-yl)methyl)-4-methoxybenzo[d]isoxazol-3-yl)-3-(7-((1-(4-(2,6-dioxopiperidin-3-yl)phenyl)piperidin-4-yl)methyl)-2,7-diazaspiro[3.5]nonan-2-yl)-5-fluoro-2,6-dimethoxybenzenesulfonamide;
rac-3-(cis)-N-(6-((1H-pyrazol-1-yl)methyl)-4-methoxybenzo[d]isoxazol-3-yl)-3-((1-(3-(2,4-dioxotetrahydropyrimidin-1(2H)-yl)-1-methyl-1H-indazol-6-yl)-piperidin-4-yl)methyl)-3,7-diazabicyclo[4.2.0]octan-7-yl)-5-fluoro-2,6-dimethoxy-benzenesulfonamide;
or a pharmaceutically acceptable salt thereof.

In a seventy fourth aspect of the invention, provided is a compound of any one of the preceding aspects, in the form of a pharmaceutically acceptable salt.

In a seventy fifth aspect of the invention, provided is a compound of any one of the preceding aspects, or a pharmaceutically acceptable salt thereof, and a pharmaceutically acceptable excipient.

In a seventy sixth aspect of the invention, provided is a method of treating cancer or solid tumors in a subject in need thereof comprising administering to the subject a compound of any one of the first through seventy fourth aspects or a pharmaceutical composition of the seventy fifth aspect.

In a seventy seventh aspect of the invention, provided is a method of the seventy sixth aspect, wherein the cancer is selected from the group consisting of lung cancer, mesothelioma, bone cancer, pancreatic cancer, skin cancer, cancer of the head or neck, cutaneous or intraocular melanoma, uterine cancer, ovarian cancer, rectal cancer, cancer of the anal region, stomach cancer, hepatic carcinoma, colon cancer, breast cancer, uterine cancer, carcinoma of the fallopian tubes, carcinoma of the endometrium, carcinoma of the cervix, carcinoma of the vagina, carcinoma of the vulva, Hodgkin's disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, hematology malignancy, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, glioblastoma, brain stem glioma, pituitary adenoma, and a combination of two or more of the foregoing cancers.

In a seventy eighth aspect of the invention, provided is a method of the seventy sixth aspect, wherein the solid tumors are selected from the group consisting of breast, lung, colon, brain, prostate, stomach, pancreatic, ovarian, melanoma, endocrine, uterine, testicular, hematologic and bladder.

In a seventy ninth aspect of the invention, provided is a method of the seventy eighth aspect, wherein the solid tumors are selected from the group consisting of breast, lung, prostate, pancreatic, hematologic and ovarian.

In an eightieth aspect of the invention, provided is a method of the seventy sixth aspect, wherein the cancer is breast cancer.

In an eighty first aspect of the invention, provided is a method of the eightieth aspect, wherein the breast cancer is ER+ breast cancer.

In an eighty second aspect of the invention, provided is a method of the eightieth aspect, wherein the breast cancer is ER+ HER2− breast cancer.

In an eighty third aspect of the invention, provided is a method of the eightieth aspect, wherein the breast cancer is locally advanced or metastatic ER+ HER2− breast cancer.

In an eighty fourth aspect of the invention, provided is a method of the seventy sixth aspect, wherein the cancer is lung cancer.

In an eighty fifth aspect of the invention, provided is a method of the eighty fourth aspect, wherein the lung cancer is non-small cell lung cancer.

In an eighty sixth aspect of the invention, provided is a method of the eighty fourth aspect, wherein the lung cancer is locally advanced or metastatic non-small cell lung cancer.

In an eighty seventh aspect of the invention, provided is a method of the seventy sixth aspect, wherein the cancer is prostate cancer.

In an eighty eighth aspect of the invention, provided is a method of the eighty seventh aspect, wherein the prostate cancer is castration resistant prostate cancer.

In an eighty ninth aspect of the invention, provided is a method of the eighty seventh aspect, wherein the prostate cancer is locally advanced or metastatic castration resistant prostate cancer.

In a ninetieth aspect of the invention, provided is a method of degrading a KAT6 protein comprising contacting the KAT6 protein with a compound of any one of the first through seventy fourth aspects or a pharmaceutical composition of the seventy fifth aspect.

In a ninety first aspect of the invention, provided is a compound of the first aspect, wherein R² is H or —(C(R⁸)₂)ₙ-(5-9 membered heteroaryl) optionally substituted by halogen, C₁-C₃ alkyl, —CH₂OH, or —OH.

Compounds of the invention can be prepared using numerous preparatory reactions known in the literature. The Schemes below provide general guidance in connection with preparing the compounds of the invention. One skilled in the art would understand that the preparations shown in the Schemes can be modified or optimized using general knowledge of organic chemistry to prepare various compounds of the invention. Example synthetic methods for preparing compounds of the invention are provided in the Schemes below.

The following Examples are provided to illustrate some of the concepts described within this disclosure. While the Examples are considered to provide an embodiment, it should not be considered to limit the more general embodiments described herein.

### EXAMPLES

### General Synthetic Procedures

The compounds of the Invention may be prepared using the general procedures described below. The compounds described herein may be prepared according to the following synthetic schemes and general synthetic procedures.

Compounds of Formula (I) can be prepared as described in Scheme I. Coupling of optionally protected compounds 1-1 with compounds 1-2 where RG⁵ and RG⁶ are each independently reactive groups such as, but not limited to, halogen (e.g., F, Cl, Br, I), hydroxy, aldehyde, boronic acid, boronate ester, trialkyltin, carboxylic acid, or amine and y is an integer from 0 to 14 using appropriate synthetic methods (such as, but not limited to, S_{N}Ar reaction, Suzuki coupling, Stille coupling, Buchwald-Hartwig reaction, Mitsunobu reaction, Williamson ether synthesis, amide coupling, or reductive amination) can afford compounds 1-3. Reaction of optionally protected 1-4 with compounds 1-5 where RG⁷ and RG⁸ are each independently reactive groups such as, but not limited to, halogen (e.g., F, Cl, Br, I), hydroxy, aldehyde, boronic acid, boronate ester, trialkyltin, carboxylic acid, or amine and z is an integer from 0 to 14 and the sum of y and z is an integer from 1 to 14 using appropriate synthetic methods (such as, but not limited to, S_{N}Ar reaction, Suzuki coupling, Stille coupling, Buchwald-Hartwig reaction, Mitsunobu reaction, Williamson ether synthesis, amide coupling, or reductive amination) can afford compounds 1-6. Coupling of compounds 1-3 and 1-6 using appropriate synthetic methods (such as, but not limited to, S_{N}Ar reaction, Suzuki coupling, Stille coupling, Buchwald-Hartwig reaction, Mitsunobu reaction, Williamson ether synthesis, amide coupling, or reductive amination) can afford compounds 1-7.

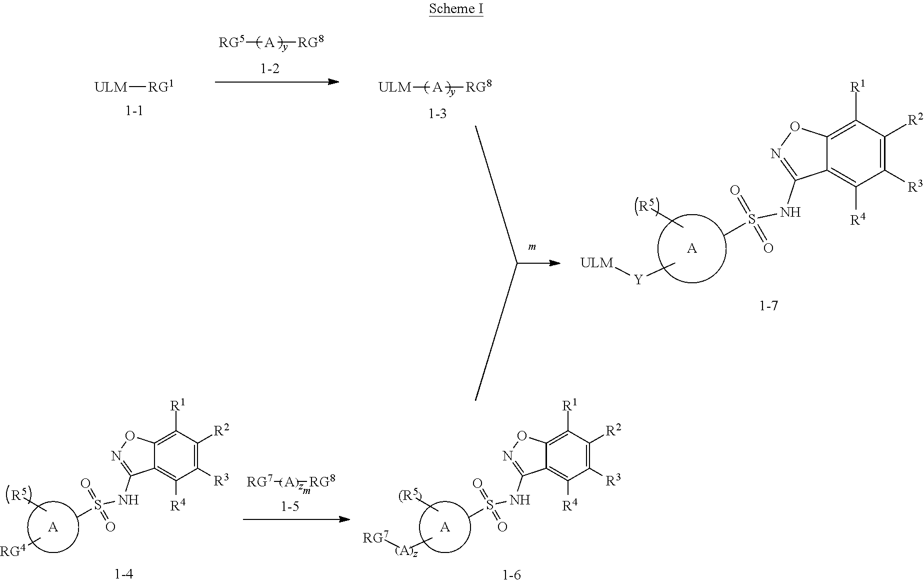

Compounds of Formula (I) can be prepared as described in Scheme II. Cleavage of acetals 2-1 where each R^{z} is independently a C₁-C₆ alkyl group or where both R^{z} can be taken together with the atoms to which they are both attached form a monocyclic heterocycloalkyl under standard conditions such as in the presence of an acid (e.g., toluenesulfonic acid or trifluoroacetic acid) can afford compounds 2-2. Coupling of compounds 2-3 where X^{e} is a halogen (e.g., F, Cl, Br, or I) or pseudohalogen (e.g., OMs or OTf) with compounds 2-4 where A₂a is 3-11 membered diazaheterocyclyl and P¹ is an appropriate nitrogen protecting group (e.g., Boc, Cbz, Bn, PMB, or acetyl) under standard S_{N}Ar conditions optionally in the presence of a base (e.g., DIPEA) or under standard Buchwald-Hartwig amination conditions (e.g., in the presence of a palladium catalyst, such as BrettPhos Pd G3, and a base, such as sodium tert-butoxide) and subsequent deprotection can afford compounds 2-5. Reaction of compounds 2-2 and amines 2-5 under standard conditions for reductive amination (e.g., in the presence of a reducing agent such as sodium triacetoxy-borohydride or sodium cyanoborohydride and optionally an acid, such as acetic acid) can afford compounds 2-6.

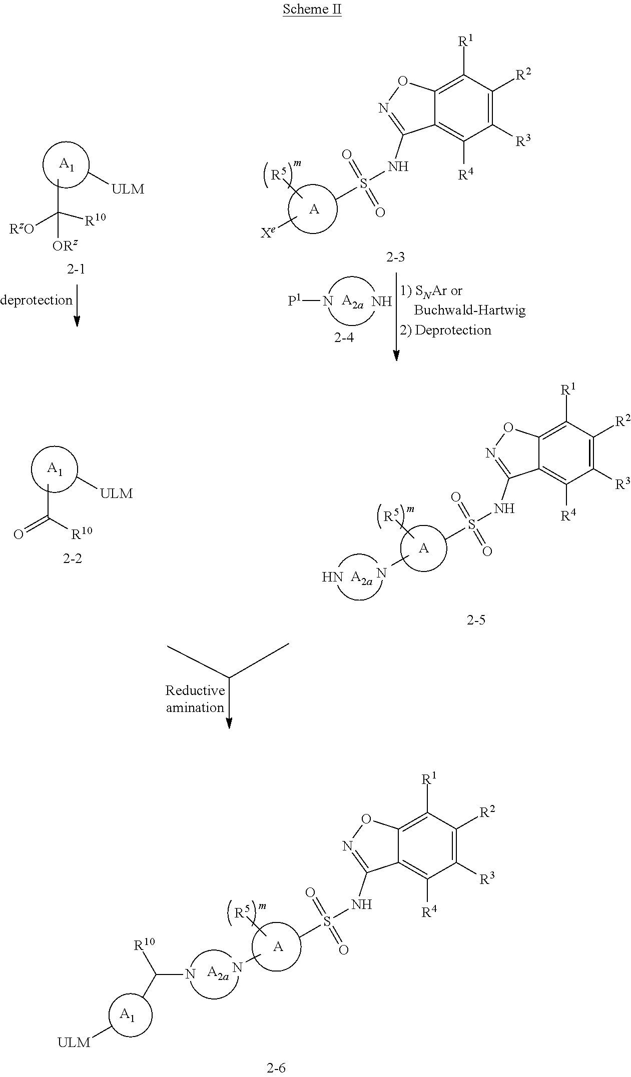

Compounds of Formula (I) can be prepared as described in Scheme III. Coupling of compounds 2-3 where X^{e} is a halogen (e.g., F, Cl, Br, or I) or pseudohalogen (e.g., OMs or OTf) with compounds 3-1 where A₂b is 3-11 membered azaheterocyclyl, P¹ is an appropriate nitrogen protecting group (e.g., Boc, Cbz, Bn, PMB, or acetyl), and M¹ is a boronic acid, boronate ester, potassium trifluoroborate, or an appropriately substituted metal, such as Sn(Bu)₃, Sn(Me)₃, ZnCl, ZnI, or ZnBr under standard Suzuki conditions (e.g., in the presence of a palladium catalyst, such as SPhos Pd G2, and a base, such as potassium carbonate) or under standard Stille conditions (e.g., in the presence of a palladium catalyst, such as SPhos Pd G2 or tetrakis(triphenylphosphine)palladium(O)) or under standard Negishi cross-coupling conditions (e.g., in the presence of a palladium catalyst, such as SPhos Pd G2, tetrakis-(triphenylphosphine)palladium(0), or [1,1′-bis(diphenylphosphino)ferrocene]dichloro-palladium(II)) and subsequent deprotection can afford compounds 3-2. Reaction of compounds 2-2 and amines 3-2 under standard conditions for reductive amination (e.g., in the presence of a reducing agent such as sodium triacetoxy-borohydride or sodium cyanoborohydride and optionally an acid, such as acetic acid) can afford compounds 3-3.

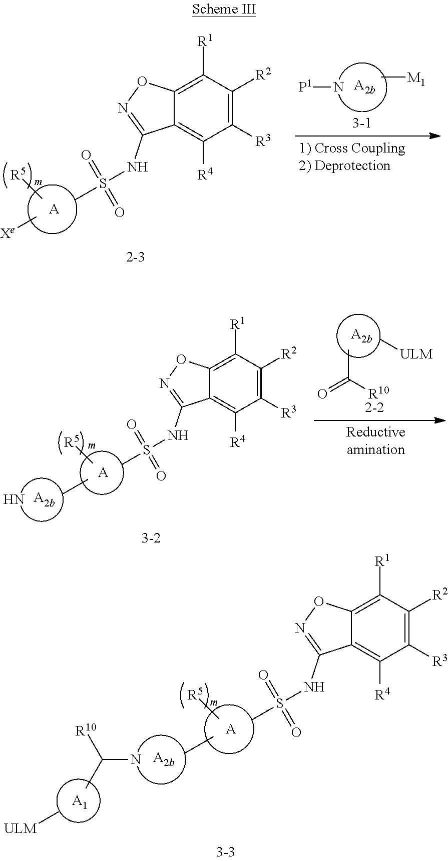

Compounds of Formula (I) can be prepared as described in Scheme IV. Coupling of compounds 2-3 where X^{e} is a halogen (e.g., F, Cl, Br, or I) or pseudohalogen (e.g., OMs or OTf) with compounds 4-1 where A₂b is 3-11 membered azaheterocyclyl and P² is an appropriate alcohol protecting group (e.g., TBS, TMS) under standard S_{N}Ar conditions optionally in the presence of a base (e.g., DIPEA) or under standard Buchwald-Hartwig amination conditions (e.g., in the presence of a palladium catalyst, such as BrettPhos Pd G3, and a base, such as sodium tert-butoxide) and subsequent deprotection can afford compounds 4-2. Oxidation of alcohols 4-2 under standard conditions such as in the presence of an oxidant (e.g., Dess Martin periodinane, 2-iodoxybenzoic acid, or sulfur trioxide pyridine complex) and optionally in the presence of a base (e.g., NaHCO₃ or Et₃N) can afford compounds 4-3. Reaction of compounds 4-3 and amines 4-4 where A_{1b} is 3-11 membered azaheterocyclyl under standard conditions for reductive amination (e.g., in the presence of a reducing agent such as sodium triacetoxy-borohydride or sodium cyanoborohydride and optionally an acid, such as acetic acid) can afford compounds 4-5.

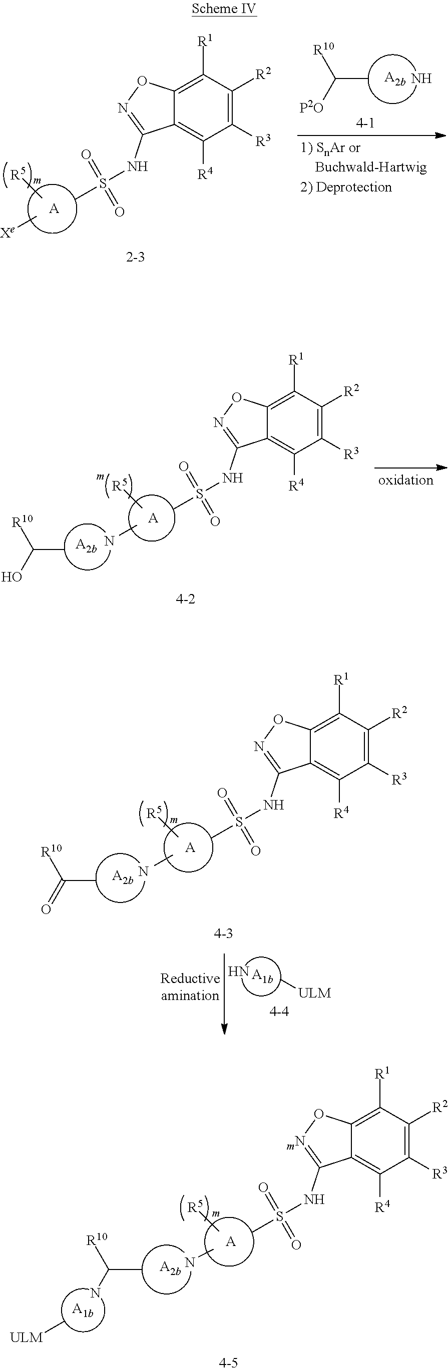

Compounds of Formula (I) can be prepared as described in Scheme V. Coupling of compounds 2-3 with compounds 5-1 where A₂c is an aryl optionally substituted with 0-6 R^{1a} or R^{1b} groups, or heteroaryl optionally substituted with 0-6 R^{1a} or R^{1b} groups and M¹ is a boronic acid, boronate ester, potassium trifluoroborate, or an appropriately substituted metal, such as Sn(Bu)₃, Sn(Me)₃, ZnI, ZnCl, or ZnBr under standard Suzuki conditions (e.g., in the presence of a palladium catalyst, such as SPhos Pd G2, and a base, such as potassium carbonate) or under standard Stille conditions (e.g., in the presence of a palladium catalyst, such as SPhos Pd G2 or tetrakis(triphenylphosphine)palladium(0)) or under standard Negishi cross-coupling conditions (e.g., in the presence of a palladium catalyst, such as SPhos Pd G2, tetrakis(triphenylphosphine)palladium(0), or [1,1′-bis(diphenylphosphino)ferrocene]dichloro-palladium(II)) can afford compounds 5-2. Reaction of compounds 5-2 and amines 4-4 where A_{1b} is a 3-12 membered heterocyclyl optionally substituted with 0-6 R^{1a} or R^{1b} groups under standard reductive amination conditions (e.g., in the presence of a reducing agent, such as sodium triacetoxyborohydride or sodium cyanoborohydride, and optionally an acid, such as acetic acid, and optionally a base, such as triethylamine) can afford compounds 5-3.

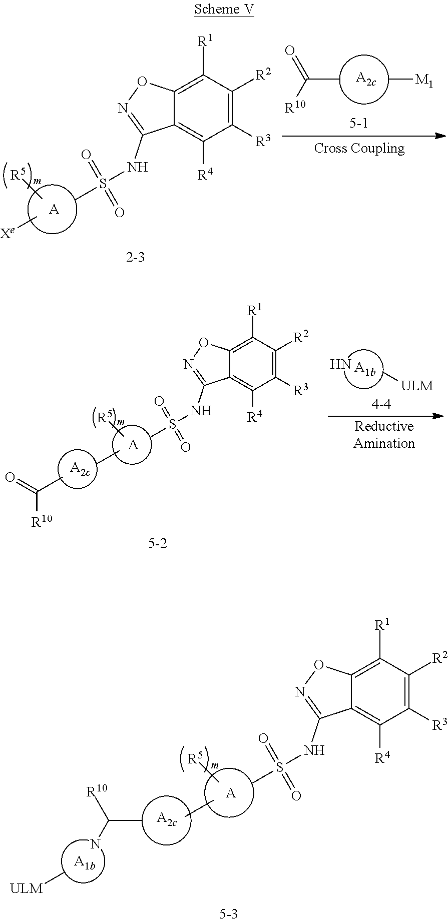

Intermediates for the synthesis of compound of Formula (I) can be prepared as described in Scheme VI. Coupling of compounds 6-1 where X^{a} is a halogen (e.g., Br or I) or pseudohalogen (e.g., OTf or OMs) with pyridines 6-2, where P³ is an appropriate protecting group (e.g., Bn or PMB) and M² is a boronic acid, boronate ester, potassium trifluoroborate, or an appropriately substituted metal, such as Sn(Bu)₃, Sn(Me)₃, ZnBr, ZnI, or ZnCl under standard Suzuki cross-coupling conditions (e.g., in the presence of a palladium catalyst, such as SPhos Pd G2) or standard Stille conditions (e.g., in the presence of a palladium (0) catalyst, such as tetrakis(triphenylphosphine)palladium (0)) or standard Negishi conditions (e.g., in the presence of a palladium catalyst, such as tetrakis(triphenylphosphine) palladium (0) or [1,1′-bis(diphenylphosphino)ferrocene]dichloropalladium(II)), can afford compounds 6-3. Coupling of compounds 6-3 where X^{b} is a halogen (e.g., F, Cl or Br) or pseudohalogen (e.g., OTf or OMs) with 6-4 where Ala is an optionally protected 3-7 membered cycloalkyl group, a 4-10-membered heterocycloalkyl group, an aryl group, or a heteroaryl group that is optionally substituted with one or more reactive groups such as, but not limited to, halogen (e.g., F, Cl, Br, I), hydroxy, aldehyde, boronic acid, boronate ester, trialkyltin, carboxylic acid, or amine under standard Suzuki (e.g., in the presence of a palladium catalyst, such as SPhos Pd G2) or standard Buchwald-Hartwig cross-coupling (e.g., in the presence of a palladium catalyst, such as BrettPhos Pd G4, and a base, such as sodium tert-butoxide) or standard S_{N}Ar reaction conditions (e.g., in the presence of a base, such as K₂CO₃, or an acid, such asp-toluenesulfonic acid) can afford compounds 6-5. Compounds 6-5 can be hydrogenated under standard conditions, such as exposure to H₂ or D₂ gas in the presence of a transition metal catalyst (e.g., palladium on charcoal or platinum(IV) oxide) to afford compounds 6-6.

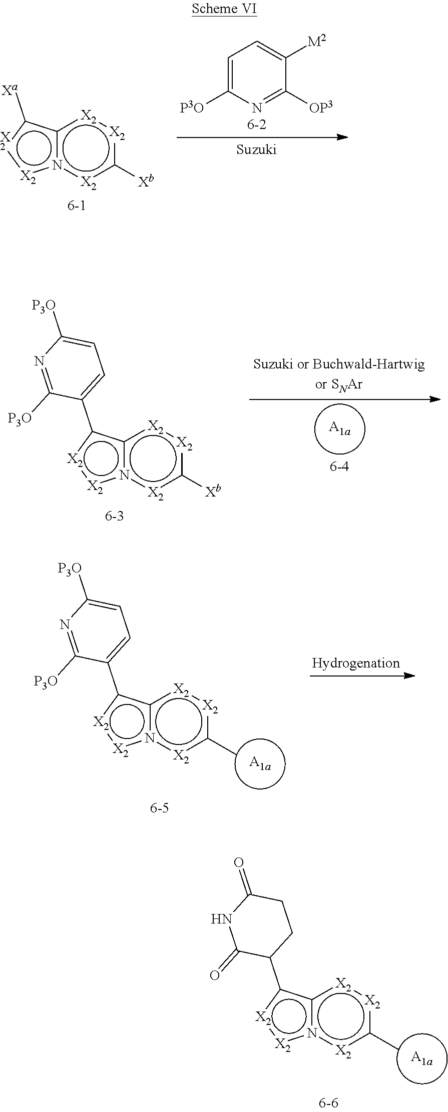

Intermediates for the synthesis of compounds of Formula (I) can be prepared as described in Scheme VII. Coupling of compounds 7-1 with compounds 7-2, where P⁴ is an appropriate protecting group (e.g., Bn or PMB) under standard Ullmann cross-coupling conditions (e.g., in the presence of a copper catalyst, such as copper acetate, and a ligand, such as tetramethyl phenanthroline) can afford dihydrouracils 7-3. Coupling of 7-3 with 6-4 under standard Suzuki (e.g., in the presence of a palladium catalyst, such as SPhos Pd G2) or standard Buchwald-Hartwig cross-coupling (e.g., in the presence of a palladium catalyst, such as BrettPhos Pd G4, and a base, such as sodium tert-butoxide) or standard S_{N}Ar reaction conditions (e.g., in the presence of a base, such as K₂CO₃ or an acid, such asp-toluenesulfonic acid) can afford compounds 7-5. Compounds 7-5 can be deprotected under standard conditions, such as in the presence of an acid (e.g., TFA) to afford compounds 7-6.

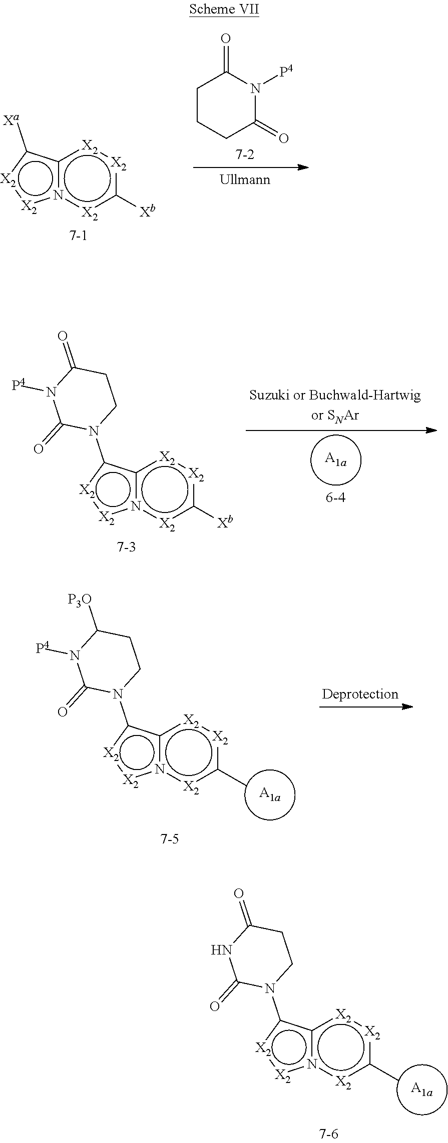

Intermediates for the synthesis of compound of Formula (I) can be prepared as described in Scheme VIII. Coupling of compounds 8-1 with pyridines 6-2, under standard Suzuki cross-coupling conditions (e.g., in the presence of a palladium catalyst, such as SPhos Pd G2) or standard Stille conditions (e.g., in the presence of a palladium (0) catalyst, such as tetrakis(triphenylphosphine)palladium (0)) or standard Negishi conditions (e.g., in the presence of a palladium catalyst, such as tetrakis(triphenylphosphine) palladium (0) or [1,1′-bis(diphenylphosphino)ferrocene]dichloropalladium(II)), can afford compounds 8-2. Coupling of compounds 8-2 with 6-4 under standard Suzuki (e.g., in the presence of a palladium catalyst, such as SPhos Pd G2) or standard Buchwald-Hartwig cross-coupling (e.g., in the presence of a palladium catalyst, such as BrettPhos Pd G4, and a base, such as sodium tert-butoxide) or standard S_{N}Ar reaction conditions (e.g., in the presence of a base, such as K₂CO₃, or an acid, such as p-toluenesulfonic acid) can afford compounds 8-3. Compounds 8-3 can be hydrogenated under standard conditions, such as exposure to H₂ or D₂ gas in the presence of a transition metal catalyst (e.g., palladium on charcoal or platinum(IV) oxide) to afford compounds 8-4.

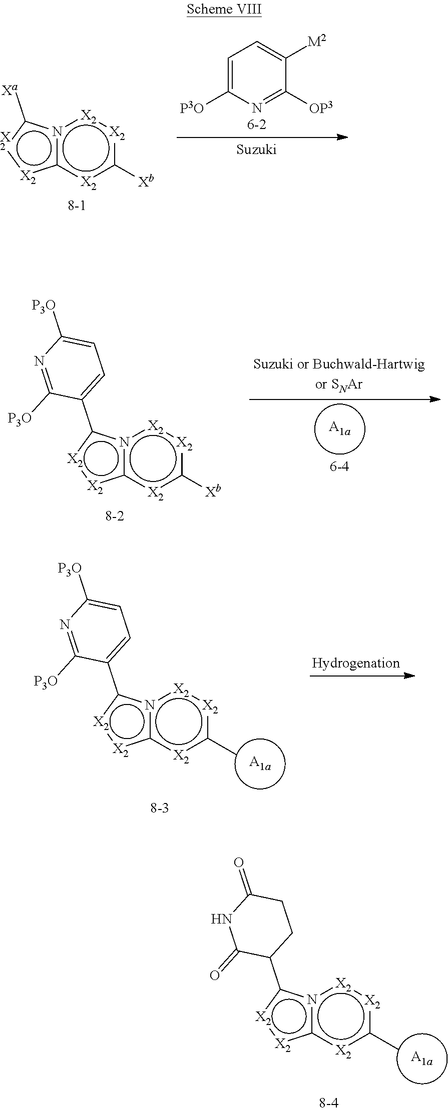

Intermediates for the synthesis of compounds of Formula (I) can be prepared as described in Scheme IX. Coupling of compounds 9-1 with compounds 7-2 under standard Ullmann cross-coupling conditions (e.g., in the presence of a copper catalyst, such as copper acetate, and a ligand, such as tetramethyl phenanthroline) can afford dihydrouracils 9-2. Coupling of 9-2 with 6-4 under standard Suzuki (e.g., in the presence of a palladium catalyst, such as SPhos Pd G2) or standard Buchwald-Hartwig cross-coupling (e.g., in the presence of a palladium catalyst, such as BrettPhos Pd G4, and a base, such as sodium tert-butoxide) or standard S_{N}Ar reaction conditions (e.g., in the presence of a base, such as K₂CO₃ or an acid, such asp-toluenesulfonic acid) can afford compounds 9-3. Compounds 9-3 can be deprotected under standard conditions, such as in the presence of an acid (e.g., TFA) to afford compounds 9-4.

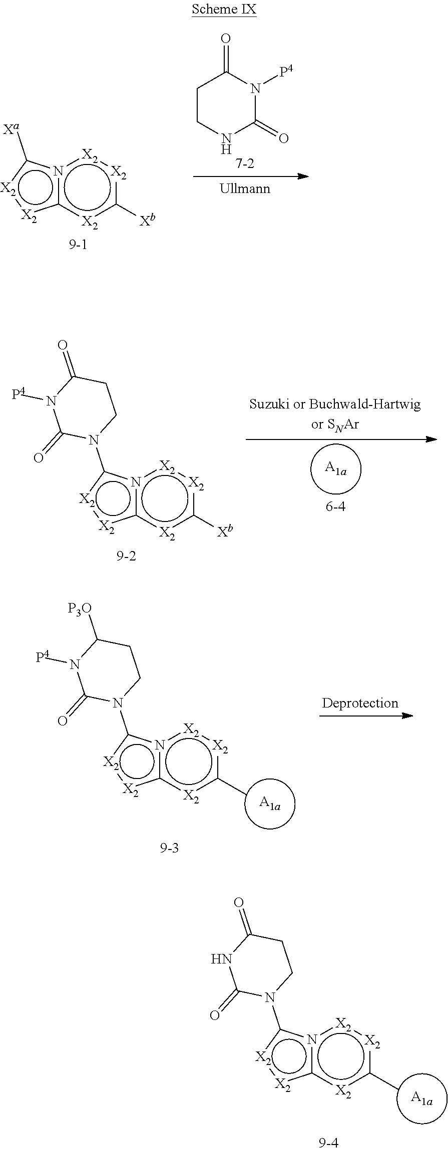

Intermediates for the synthesis of compounds of Formula (I) can also be prepared as previously described (WO2023019166A1; WO2022195454A1; WO2021053555A1; WO2024255810A1; CN120025316A; WO2017197046A1; CN115557933A; WO2021053555A1; WO2022125790A1; CN120025333A; WO2017197051A1; Norris, S., et al. J. Med. Chem. 2023, 66, 16388-16409; Teng, M. et al. J. Med. Chem. 2022, 65, 747-756; Lejava, A., et al. ACS Med. Chem. Lett. 2025, 16, 89-95; Actis, et al. J. Med Chem. 2025, 68, 3591-3611.).

### Example 1. N-(6-((1H-Pyrazol-1-yl)methyl)-4-methoxybenzo[d]isoxazol-3-yl)-3-(7-(((3S)-1-(1-(2,6-dioxopiperidin-3-yl)-3-methyl-1H-indazol-5-yl)pyrrolidin-3-yl)methyl)-2,7-diazaspiro[3.5]nonan-2-yl)benzenesulfonamide

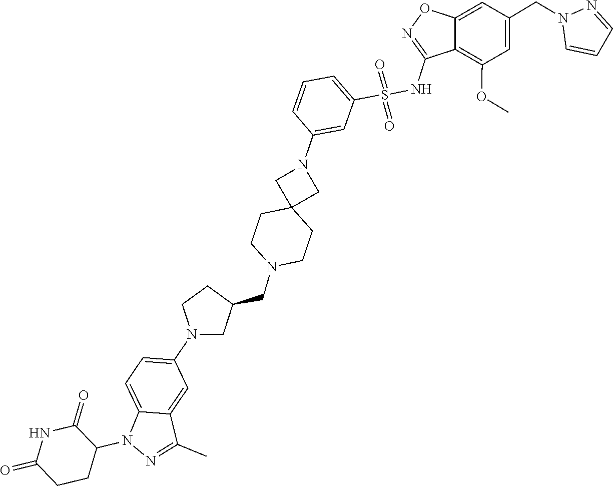 [SMILES: COc1cc(Cn2cccn2)cc2onc(NS(=O)(=O)c3cccc(N4CC5(CCN(C[C@@H]6CCN(c7ccc8c(c7)c(C)nn8C7CCC(=O)NC7=O)C6)CC5)C4)c3)c12]

### Step 1. tert-Butyl (R)-3-((benzyloxy)methyl)pyrrolidine-1-carboxylate

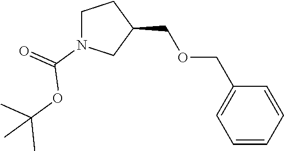 [SMILES: CC(C)(C)OC(=O)N1CC[C@@H](COCc2ccccc2)C1]

To a solution of sodium hydride (60% in mineral oil, 159 mg, 3.97 mmol) in THF (5 mL) at 0° C. was added a solution of tert-butyl (R)-3-(hydroxymethyl)pyrrolidine-1-carboxylate (400 mg, 1.99 mmol) in THF (5 mL). The reaction was stirred for 0.5 h at 0° C. Benzyl bromide (0.350 mL, 2.98 mmol) was added and the reaction was stirred for 2 h at room temperature. The reaction mixture was quenched with sat. NH₄Cl (aq) and extracted with EtOAc (3×15 mL). The combined organic layers were washed with brine, dried over MgSO₄, filtered, and concentrated. The crude material was purified by silica gel chromatography (0-25% EtOAc/hexanes) to afford the title compound (192 mg, 0.659 mmol, 33.1%) as a clear, colorless oil. LCMS calc. for C₁₇H₂₆NO₃ [M+H]⁺: m/z=292.2; Found 292.1.

### Step 2. (R)-3-((Benzyloxy)methyl)pyrrolidine

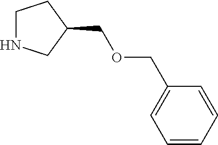 [SMILES: c1ccc(COC[C@@H]2CCNC2)cc1]

To a solution of tert-butyl (R)-3-((benzyloxy)methyl)pyrrolidine-1-carboxylate (659 mg, 2.26 mmol) in 1,4-dioxane (5 mL) was added hydrochloric acid (4N in dioxane, 8.48 mL, 33.9 mmol). The reaction was stirred for 1 h. The reaction mixture was concentrated and carried forward without further purification. LCMS calc. for C₁₂H₁₈NO [M+H]⁺: m/z=192.1; Found 192.1.

### Step 3. 1-(2,6-bis(Benzyloxy)pyridin-3-yl)-5-bromo-3-methyl-1H-indazole

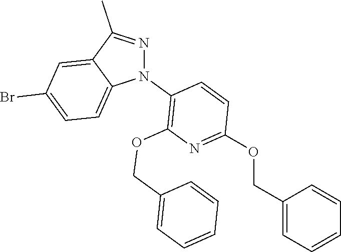 [SMILES: Cc1nn(-c2ccc(OCc3ccccc3)nc2OCc2ccccc2)c2ccc(Br)cc12]

A mixture of 2,6-bis(benzyloxy)-3-(4,4,5,5-tetramethyl-1,3,2-dioxaborolan-2-yl)pyridine (1.66 g, 3.98 mmol), 5-bromo-3-methyl-1H-indazole (700 mg, 3.32 mmol), cesium carbonate (1.30 g, 3.98 mmol), copper(II) acetate (120 mg, 0.663 mmol), and 1,10-phenanthroline (120 mg, 0.663 mmol) in DCE (20 mL) was stirred at 70° C., open to air, for 16 h. The reaction mixture was filtered through Celite and the filtrate was concentrated and purified by silica gel chromatography (0-25% EtOAc/hexanes) to afford the title compound (999 mg, 2.00 mmol, 60.2%). LCMS calc. for C₂₇H₂₃BrN₃O₂ [M+H]⁺: m/z=500.1, 502.1; Found 500.0, 501.9.

### Step 4. (R)-5-(3-((Benzyloxy)methyl)pyrrolidin-1-yl)-1-(2,6-bis(benzyloxy)pyridin-3-yl)-3-methyl-1H-indazole

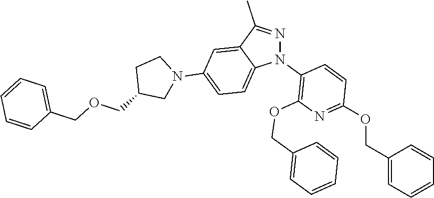 [SMILES: Cc1nn(-c2ccc(OCc3ccccc3)nc2OCc2ccccc2)c2ccc(N3CC[C@@H](COCc4ccccc4)C3)cc12]

A solution of 1-(2,6-bis(benzyloxy)pyridin-3-yl)-5-bromo-3-methyl-1H-indazole (200 mg, 0.400 mmol), 2-(dicyclohexylphosphino)-2′,4′,6′-tri-i-propyl-1,1′-biphenyl (38 mg, 0.080 mmol), cesium carbonate (430 mg, 1.32 mmol), (R)-3-((benzyloxy)methyl)pyrrolidine (107 mg, 0.560 mmol), and tris(dibenzylideneacetone)dipalladium(0) (36.6 mg, 0.0400 mmol) in 1,4-dioxane (4 mL) was sparged with nitrogen for 5 min. The reaction was stirred under a nitrogen atmosphere for 16 h at 110° C. The reaction mixture was cooled to room temperature and diluted with water (10 mL) and MTBE (10 mL). The aqueous layer was extracted with MTBE (3×5 mL). The combined organic layers were washed with brine, dried over MgSO₄, filtered, and concentrated. The crude material was purified by silica gel chromatography (0-25% EtOAc/hexanes) to afford the title compound (178 mg, 0.291 mmol, 73.0%) as a yellow solid. LCMS calc. for C₃₉H₃₉N₄O₃ [M+H]⁺: m/z=611.3; Found 611.2.

### Step 5. 3-(5-((R)-3-(Hydroxymethyl)pyrrolidin-1-yl)-3-methyl-1H-indazol-1-yl)piperidine-2,6-dione

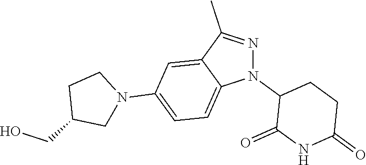 [SMILES: Cc1nn(C2CCC(=O)NC2=O)c2ccc(N3CC[C@@H](CO)C3)cc12]

A solution of (R)-5-(3-((benzyloxy)methyl)pyrrolidin-1-yl)-1-(2,6-bis(benzyloxy)-pyridin-3-yl)-3-methyl-1H-indazole (400 mg, 0.655 mmol), acetic acid (0.075 mL, 1.3 mmol) and palladium on carbon (10% wt, 139 mg, 0.131 mmol) in methanol (2.5 mL) and THF (2.5 mL) was sparged with hydrogen for 5 min. The reaction was stirred overnight under a hydrogen atmosphere (balloon) then filtered through Celite and concentrated. The crude material was purified by silica gel chromatography (50-100% EtOAc/hexanes then 0-20% MeOH/EtOAc) to afford the title compound (107 mg, 0.313 mmol, 46.0% yield). LCMS calc. for C₁₈H₂₃N₄O₃ [M+H]⁺: m/z=343.2; Found 343.1.

### Step 6. N-(6-((1H-Pyrazol-1-yl)methyl)-4-methoxybenzo[d]isoxazol-3-yl)-3-(7-(((3S)-1-(1-(2,6-dioxopiperidin-3-yl)-3-methyl-1H-indazol-5-yl)pyrrolidin-3-yl)methyl)-2, 7-diazaspiro[3.5]nonan-2-yl)benzenesulfonamide

To a solution of 3-(6-((R)-3-(hydroxymethyl)pyrrolidin-1-yl)-1-methyl-1H-indazol-3-yl)piperidine-2,6-dione (5.4 mg, 0.016 mmol) and triethylamine (0.020 mL, 0.14 mmol) in DMSO (0.30 mL) was added a solution of sulfurtrioxide pyridine complex (7.5 mg, 0.047 mmol) in DMSO (0.30 mL). The reaction was stirred for 1 h. A solution of N-(6-((1H-pyrazol-1-yl)methyl)-4-methoxybenzo[d]isoxazol-3-yl)-3-(2,7-diazaspiro[3.5]nonan-2-yl)benzenesulfonamide (which can be prepared as described in WO2023245150A1) (8.0 mg, 0.016 mmol) in MeCN (0.20 mL) and DMSO (0.30 mL) was added followed by acetic acid (0.014 mL, 0.24 mmol). The reaction was stirred for 0.5 h. Sodium triacetoxyborohydride (13 mg, 0.063 mmol) was added and the reaction was stirred for 1 h. The reaction was quenched with water, diluted with MeCN, filtered, and purified by prep-HPLC on a C18 column (21.7-41.7% MeCN/0.2% TFA (aq)) to afford the title compound (2.3 mg, 0.0022 mmol, 14%) as a white solid. LCMS calc. for C₄₃H₄₉N₁₀O₆S [M+H]⁺: m/z=833.4; Found 833.2.

### Example 2. N-(6-((1H-Pyrazol-1-yl)methyl)-4-methoxybenzo[d]isoxazol-3-yl)-3-(7-((1-(1-(2,6-dioxopiperidin-3-yl)-3-methyl-2-oxo-2,3-dihydro-1H-benzo[d]imidazol-5-yl)piperidin-4-yl)methyl)-2,7-diazaspiro[3.5]nonan-2-yl)benzenesulfonamide

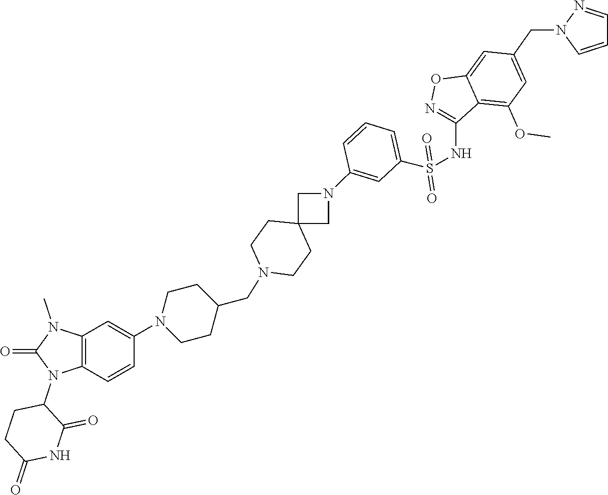 [SMILES: COc1cc(Cn2cccn2)cc2onc(NS(=O)(=O)c3cccc(N4CC5(CCN(CC6CCN(c7ccc8c(c7)n(C)c(=O)n8C7CCC(=O)NC7=O)CC6)CC5)C4)c3)c12]

To a solution of 1-(1-(2,6-dioxopiperidin-3-yl)-3-methyl-2-oxo-2,3-dihydro-1H-benzo[d]imidazol-5-yl)piperidine-4-carbaldehyde (5.1 mg, 0.014 mmol) and N-(6-((1H-pyrazol-1-yl)methyl)-4-methoxybenzo[d]isoxazol-3-yl)-3-(2,7-diazaspiro[3.5]nonan-2-yl)benzenesulfonamide (7.0 mg, 0.014 mmol) in MeCN (0.15 mL) and DMSO (0.60 mL) was added acetic acid (0.012 mL, 0.21 mmol). The reaction was stirred for 0.5 h. Sodium triacetoxyborohydride (12 mg, 0.055 mmol) was added and the reaction was stirred for 1 h. The reaction was quenched with water, diluted with MeCN, filtered, and purified by prep-HPLC on a C18 column (18.1-38.1% MeCN/0.2% TFA (aq)) to afford the title compound as a TFA salt (5.0 mg, 0.0046 mmol, 33%), a white solid. ¹H NMR (400 MHz, DMSO) δ 11.10 (s, 1H), 10.83 (s, 1H), 7.89 (d, J=2.2 Hz, 1H), 7.51 (d, J=1.8 Hz, 1H), 7.40 (t, J=7.9 Hz, 1H), 7.27 (d, J=7.7 Hz, 1H), 7.12-6.93 (m, 3H), 6.84 (s, 1H), 6.76 (s, 2H), 6.67 (dd, J=8.2, 2.3 Hz, 1H), 6.31 (t, J=2.1 Hz, 1H), 5.45 (s, 2H), 5.33 (dd, J=12.9, 5.4 Hz, 1H), 3.87 (s, 3H), 3.75-3.67 (m, 6H), 3.55-3.41 (m, 4H), 3.33 (s, 3H), 3.14-2.81 (m, 5H), 2.78-2.58 (m, 2H), 2.13 (d, J=13.8 Hz, 2H), 2.06-1.83 (m, 6H), 1.56-1.34 (m, 2H). LCMS calc. for C₄₄H₅₁N₁₀O₇S [M+H]⁺: m/z=863.4; Found 863.4.

### Example 3. N-(6-((1H-Pyrazol-1-yl)methyl)-4-methoxybenzo[d]isoxazol-3-yl)-3-(7-((1-(4-(2,6-dioxopiperidin-3-yl)phenyl)pyrrolidin-3-yl)methyl)-2,7-diazaspiro[3.5]nonan-2-yl)benzenesulfonamide

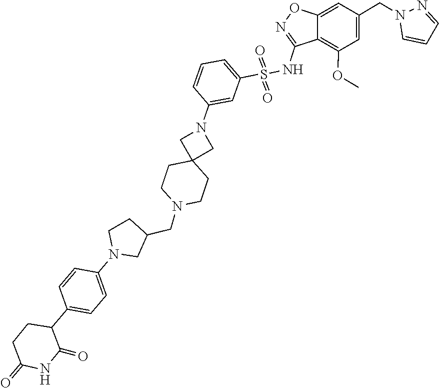 [SMILES: COc1cc(Cn2cccn2)cc2onc(NS(=O)(=O)c3cccc(N4CC5(CCN(CC6CCN(c7ccc(C8CCC(=O)NC8=O)cc7)C6)CC5)C4)c3)c12]

### Step 1. (1-(4-Bromophenyl)pyrrolidin-3-yl)methanol

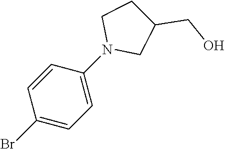 [SMILES: OCC1CCN(c2ccc(Br)cc2)C1]

A solution of 1-bromo-4-iodobenzene (1.00 g, 3.53 mmol), pyrrolidin-3-yl-methanol (393 mg, 3.89 mmol), copper(I) iodide (135 mg, 0.707 mmol), L-proline (81.4 mg, 0.707 mmol), and potassium phosphate tribasic (1.50 mg, 7.07 mmol) in DMSO (15 mL) was stirred at 80° C. for 16 h. The reaction was cooled to room temperature and filtered through Celite. The filtrate was diluted with brine (25 mL) and EtOAc (25 mL). The aqueous layer was extracted with EtOAc (3×25 mL). The combined organic layers were washed with brine, dried over MgSO₄, filtered, and concentrated to afford the title compound (871 mg, 3.40 mmol, 96.2%) as a brown solid. LCMS calc. for C₁₁H₁₅BrNO [M+H]⁺: m/z=256.0, 258.0; Found 256.0, 258.0.

### Step 2. (1-(4-(2,6-Bis(benzyloxy)pyridin-3-yl)phenyl)pyrrolidin-3-yl)methanol

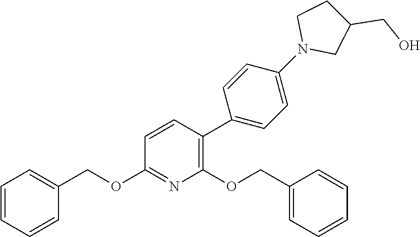 [SMILES: OCC1CCN(c2ccc(-c3ccc(OCc4ccccc4)nc3OCc3ccccc3)cc2)C1]

A solution of 2,6-bis(benzyloxy)-3-(4,4,5,5-tetramethyl-1,3,2-dioxaborolan-2-yl)pyridine (1.87 g, 4.49 mmol), [1-(4-bromophenyl)pyrrolidin-3-yl]methanol (826 mg, 3.22 mmol), cesium carbonate (2.10 g, 6.45 mmol), and [1,1′-bis(diphenylphosphino)ferrocene]-dichloropalladium(II)dichloromethane (0.290 g, 0.355 mmol) in 1,4-dioxane (35 mL) and water (4 mL) was sparged with nitrogen for 10 min then stirred for 16 h at 100° C. The reaction was cooled to room temperature, filtered, and concentrated onto Celite. The material was purified by silica gel chromatography (0-100% EtOAc/hexanes) to afford the title compound (696 mg, 1.49 mmol, 46.3%) as a tan solid. LCMS calc. for C₃₀H₃₁N₂O₃ [M+H]⁺: m/z=467.2; Found 467.1.

### Step 3. N-(6-((1H-Pyrazol-1-yl)methyl)-4-methoxybenzo[d]isoxazol-3-yl)-3-(7-((1-(4-(2,6-dioxopiperidin-3-yl)phenyl)pyrrolidin-3-yl)methyl)-2, 7-diazaspiro[3.5]nonan-2-yl)benzenesulfonamide

The title compound was synthesized according to procedures analogous to Example 1, Steps 5-6. LCMS calc. for C₄₁H₄₇N₈O₆S [M+H]⁺: m/z=779.3; Found 779.1.

### Example 4. N-(6-((1H-Pyrazol-1-yl)methyl)-4-methoxybenzo[d]isoxazol-3-yl)-3-(7-((1-(3-(2,6-dioxopiperidin-3-yl)-1-methyl-1H-pyrrolo[3,2-c]pyridin-6-yl)piperidin-4-yl)methyl)-2,7-diazaspiro[3.5]nonan-2-yl)benzenesulfonamide

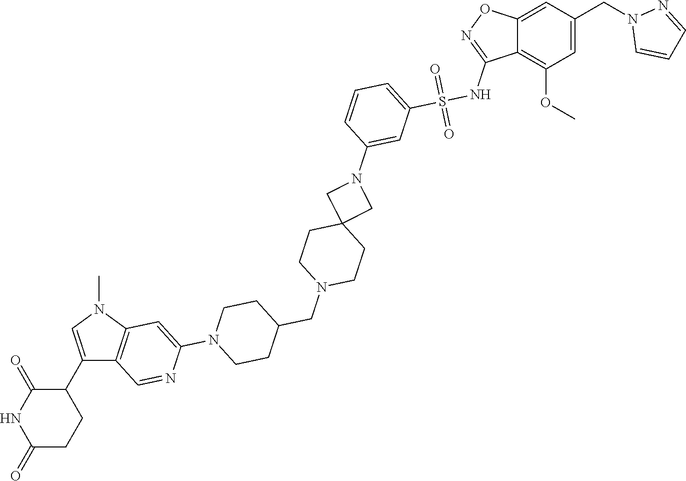 [SMILES: COc1cc(Cn2cccn2)cc2onc(NS(=O)(=O)c3cccc(N4CC5(CCN(CC6CCN(c7cc8c(cn7)c(C7CCC(=O)NC7=O)cn8C)CC6)CC5)C4)c3)c12]

### Step 1. 6-(4-(Dimethoxymethyl)piperidin-1-yl)-1-methyl-1H-pyrrolo[3,2-c]pyridine

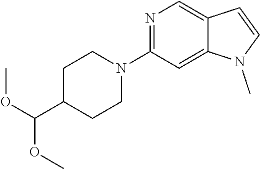 [SMILES: COC(OC)C1CCN(c2cc3c(ccn3C)cn2)CC1]

A solution of 6-bromo-1-methyl-1H-pyrrolo[3,2-c]pyridine (1.98 g, 9.37 mmol), 4-(dimethoxymethyl)piperidine (2.24 g, 14.1 mmol), potassium tert-butoxide (1N in THF, 2.63 g, 23.4 mmol), RuPhos Pd G3 (CAS 1445085-77-7, 392 mg, 0.468 mmol), and RuPhos (CAS 787618-22-8, 437 mg, 0.937 mmol) in 1,4-dioxane (25 mL) was stirred at 100° C. under a nitrogen atmosphere for 4 h. The mixture was cooled to room temperature, poured into cold water (200 mL), and extracted with EtOAc (100 mL×3). The combined organic layers were dried over Na₂SO₄, filtered, and concentrated. The crude material was purified by silica gel chromatography (0-75% EtOAc/heptanes) to afford the title compound (2.50 g, 8.64 mmol, 92.2%) as a yellow solid. LCMS calc. for C₁₆H₂₄N₃O₂ [M+H]⁺: m/z=290.2; Found 290.5.

### Step 2. 3-(2,6-Bis(benzyloxy)pyridin-3-yl)-6-(4-(dimethoxymethyl)piperidin-1-yl)-1-methyl-1H-pyrrolo[3,2-c]pyridine

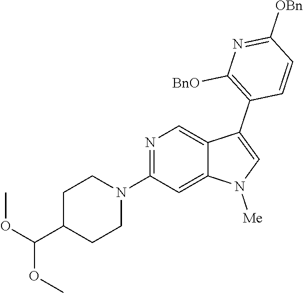 [SMILES: COC(OC)C1CCN(c2cc3c(cn2)c(-c2ccc(OCc4ccccc4)nc2OCc2ccccc2)cn3C)CC1]

A solution of 2,6-bis(benzyloxy)-3-iodopyridine (3.38 g, 8.10 mmol), 6-(4-(dimethoxymethyl)piperidin-1-yl)-1-methyl-1H-pyrrolo[3,2-c]pyridine (1.88 g, 6.48 mmol), palladium(II) acetate (0.146 g, 0.648 mmol), potassium carbonate (2.69 g, 19.5 mmol), tetrabutylammonium bromide (0.418 g, 1.30 mmol), and triphenylphosphine (0.170 g, 0.648 mmol) in DMSO (18 mL) was stirred at 120° C. for 18 h. The reaction was cooled to room temperature, diluted with EtOAc (250 mL), washed with water (75 mL) and brine (75 mL), dried over Na₂SO₄, filtered, and concentrated. The crude material was purified by silica gel chromatography (0-50% EtOAc/heptanes) to afford the title compound (3.00 g, 5.18 mmol, 79.8%) as a yellow solid. LCMS calc. for C₃₅H₃₉N₄O₄ [M+H]⁺: m/z=579.3; Found 579.6.

### Step 3. 3-(6-(4-(Dimethoxymethyl)piperidin-1-yl)-1-methyl-1H-pyrrolo[3,2-c]pyridin-3-yl)piperidine-2,6-dione

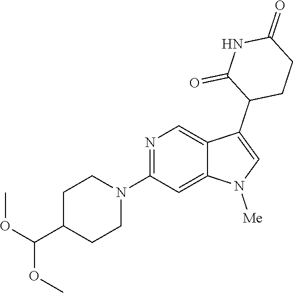 [SMILES: COC(OC)C1CCN(c2cc3c(cn2)c(C2CCC(=O)NC2=O)cn3C)CC1]

The title compound was synthesized according to procedures analogous to Example 1, Step 5. LCMS calc. for C₂₁H₂₉N₄O₄ [M+H]⁺: m/z=401.2; Found 401.4.

### Step 4. 1-(3-(2,6-Dioxopiperidin-3-yl)-1-methyl-1H-pyrrolo[3,2-c]pyridin-6-yl)piperidine-4-carbaldehyde

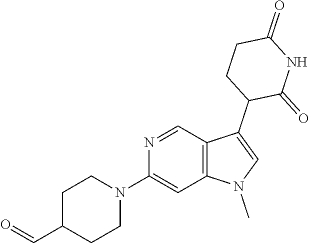 [SMILES: Cn1cc(C2CCC(=O)NC2=O)c2cnc(N3CCC(C=O)CC3)cc21]

To a solution of 3-(6-(4-(dimethoxymethyl)piperidin-1-yl)-1-methyl-1H-pyrrolo-[3,2-c]pyridin-3-yl)piperidine-2,6-dione (20.0 mg, 0.0499 mmol) in DCM (0.45 mL) and Acetone (0.18 mL) was added trifluoroacetic acid (0.153 mL, 2.00 mmol). The reaction was stirred for 2 h. The mixture was neutralized with NaHCO₃ then diluted with water and 3:1 CHCl₃/IPA. The aqueous layer was exacted with 3:1 CHCl₃/IPA (3×10 mL). The combined organic layers were washed with brine, dried over MgSO₄, filtered, and concentrated to afford the title compound (28.0 mg, 0.0790 mmol, >99%). LCMS calc. for C₁₉H₂₃N₄O₃ [M+H]⁺: m/z=355.2; Found 355.1.

### Step 5. N-(6-((1H-Pyrazol-1-yl)methyl)-4-methoxybenzo[d]isoxazol-3-yl)-3-(7-((1-(3-(2,6-dioxopiperidin-3-yl)-1-methyl-1H-pyrrolo[3,2-c]pyridin-6-yl)piperidin-4-yl)methyl)-2, 7-diazaspiro[3.5]nonan-2-yl)benzenesulfonamide

The title compound was synthesized according to procedures analogous to Example 2. ¹H NMR (400 MHz, DMSO-d₆) δ 11.03 (s, 1H), 9.26 (s, 1H), 8.60 (s, 1H), 7.89 (d, J=2.3 Hz, 1H), 7.51 (d, J=1.8 Hz, 1H), 7.40 (t, J=7.9 Hz, 1H), 7.27 (d, J=7.1 Hz, 2H), 7.01-6.93 (m, 1H), 6.84 (s, 1H), 6.76 (s, 1H), 6.68 (dd, J=8.0, 2.3 Hz, 1H), 6.62 (s, 1H), 6.31 (t, J=2.1 Hz, 1H), 5.45 (s, 2H), 4.39 (dd, J=12.4, 4.6 Hz, 1H), 4.11-3.94 (m, 2H), 3.87 (s, 4H), 3.74 (d, J=9.8 Hz, 5H), 3.66 (s, 2H), 3.51 (d, J=12.3 Hz, 3H), 3.19-2.92 (m, 5H), 2.84-2.59 (m, 2H), 2.27-2.08 (m, 4H), 2.07-1.83 (m, 4H), 1.48-1.30 (m, 2H). LCMS calc. for C₄₄H₅₁N₁₀O₆S [M+H]⁺: m/z=847.4; Found 847.2.

### Example 5. N-(6-((1H-Pyrazol-1-yl)methyl)-4-methoxybenzo[d]isoxazol-3-yl)-3-(7-((1-(2-(2,6-dioxopiperidin-3-yl)-1-oxo-1,2-dihydroisoquinolin-6-yl)piperidin-4-yl)methyl)-2,7-diazaspiro[3.5]nonan-2-yl)-2-methoxybenzenesulfonamide

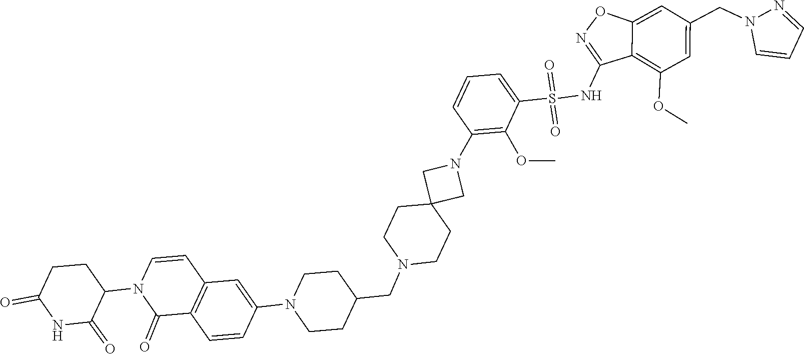 [SMILES: COc1c(N2CC3(CCN(CC4CCN(c5ccc6c(=O)n(C7CCC(=O)NC7=O)ccc6c5)CC4)CC3)C2)cccc1S(=O)(=O)Nc1noc2cc(Cn3cccn3)cc(OC)c12]

The title compound was synthesized according to procedures analogous to Example 2, using N-(6-((1H-pyrazol-1-yl)methyl)-4-methoxybenzo[d]isoxazol-3-yl)-2-methoxy-3-(2,7-diazaspiro[3.5]nonan-2-yl)benzenesulfonamide (which can be prepared as described in WO2023245150A1) and 1-(2-(2,6-dioxopiperidin-3-yl)-1-oxo-1,2-dihydroisoquinolin-6-yl)piperidine-4-carbaldehyde (which can be prepared as described in WO2022099117A1). ¹H NMR (400 MHz, DMSO-d₆) δ 11.01 (s, 1H), 10.56 (s, 1H), 7.99 (d, J=9.0 Hz, 1H), 7.89 (d, J=2.3 Hz, 1H), 7.51 (d, J=1.8 Hz, 1H), 7.27 (d, J=7.5 Hz, 1H), 7.25-7.12 (m, 3H), 6.98 (d, J=2.4 Hz, 1H), 6.86 (s, 1H), 6.83-6.75 (m, 2H), 6.47 (d, J=7.5 Hz, 1H), 6.31 (t, J=2.1 Hz, 1H), 5.45 (s, 2H), 4.00 (d, J=12.7 Hz, 2H), 3.81 (s, 3H), 3.69 (d, J=3.4 Hz, 5H), 3.61 (s, 2H), 3.55-3.40 (m, 4H), 3.15-2.76 (m, 6H), 2.73-2.55 (m, 2H), 2.11 (d, J=13.4 Hz, 3H), 2.05-1.89 (m, 3H), 1.82 (d, J=12.6 Hz, 2H), 1.30 (q, J=12.4 Hz, 2H). LCMS calc. for C₄₆H₅₂N₉O₈S [M+H]⁺: m/z=890.4; Found 890.2.

### Example 6. N-(6-((1H-Pyrazol-1-yl)methyl)-4-methoxybenzo[d]isoxazol-3-yl)-3-(7-((1-(1-(2,6-dioxopiperidin-3-yl)-3-methyl-1H-indazol-5-yl)piperidin-4-yl)methyl)-2,7-diazaspiro[3.5]nonan-2-yl)-2-methoxybenzenesulfonamide

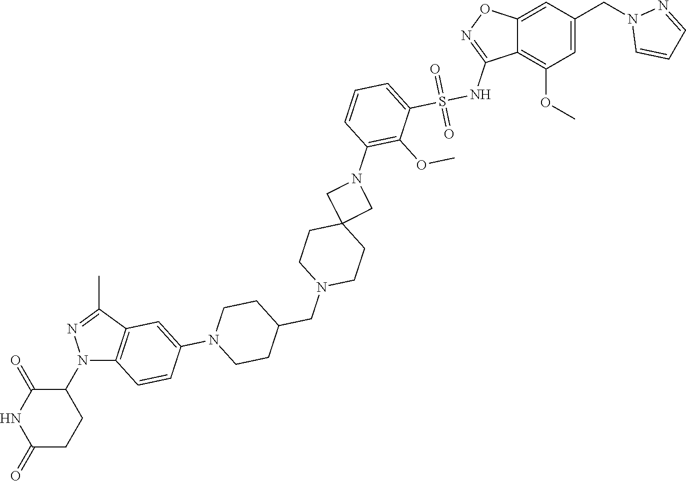 [SMILES: COc1c(N2CC3(CCN(CC4CCN(c5ccc6c(c5)c(C)nn6C5CCC(=O)NC5=O)CC4)CC3)C2)cccc1S(=O)(=O)Nc1noc2cc(Cn3cccn3)cc(OC)c12]

### Step 1. 3-(5-(4-(Dimethoxymethyl)piperidin-1-yl)-3-methyl-1H-indazol-1-yl)piperidine-2,6-dione

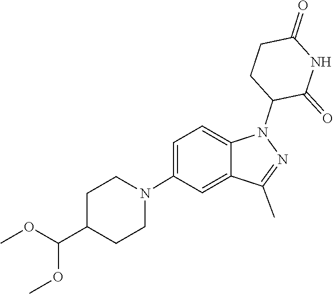 [SMILES: COC(OC)C1CCN(c2ccc3c(c2)c(C)nn3C2CCC(=O)NC2=O)CC1]

The title compound was synthesized according to procedures analogous to Example 1, Steps 4-5, using 4-(dimethoxymethyl)piperidine. LCMS calc. for C₂₁H₂₉N₄O₄ [M+H]⁺: m/z=401.2; Found 401.1.

### Step 2. 1-(1-(2,6-Dioxopiperidin-3-yl)-3-methyl-1H-indazol-5-yl)piperidine-4-carbaldehyde

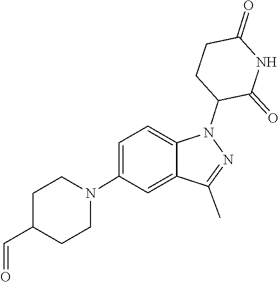 [SMILES: Cc1nn(C2CCC(=O)NC2=O)c2ccc(N3CCC(C=O)CC3)cc12]

The title compound was synthesized according to procedures analogous to Example 4, Step 4. LCMS calc. for C₁₉H₂₃N₄O₃ [M+H]⁺: m/z=355.2; Found 355.0.

### Step 3. N-(6-((1H-Pyrazol-1-yl)methyl)-4-methoxybenzo[d]isoxazol-3-yl)-3-(7-((1-(1-(2,6-dioxopiperidin-3-yl)-3-methyl-1H-indazol-6-yl)piperidin-4-yl)methyl)-2, 7-diazaspiro[3.5]nonan-2-yl)-2-methoxybenzenesulfonamide

The title compound was synthesized according to procedures analogous to Example 2. ¹H NMR (400 MHz, DMSO-d₆) δ 11.06 (s, 1H), 10.56 (s, 1H), 7.89 (d, J=2.3 Hz, 1H), 7.51 (d, J=1.9 Hz, 1H), 7.22 (dd, J=8.0, 1.6 Hz, 1H), 7.16 (t, J=7.9 Hz, 1H), 6.86 (s, 1H), 6.83-6.73 (m, 2H), 6.31 (t, J=2.1 Hz, 1H), 5.78-5.64 (m, 1H), 5.46 (s, 2H), 3.81 (s, 4H), 3.76-3.65 (m, 8H), 3.62 (s, 3H), 3.50 (d, J=12.0 Hz, 3H), 3.19-2.64 (m, 8H), 2.49-2.43 (m, 3H), 2.32-2.05 (m, 4H), 2.03-1.86 (m, 4H), 1.61-1.42 (m, 2H). LCMS calc. for C₄₅H₅₃N₁₀O₇S [M+H]⁺: m/z=877.4; Found 877.2.

### Example 7. N-(6-((1H-Pyrazol-1-yl)methyl)-4-methoxybenzo[d]isoxazol-3-yl)-3-(7-((1-(3-(2,6-dioxopiperidin-3-yl)-1-methyl-1H-indazol-6-yl)piperidin-4-yl)methyl)-2,7-diazaspiro[3.5]nonan-2-yl)-2-methoxybenzenesulfonamide

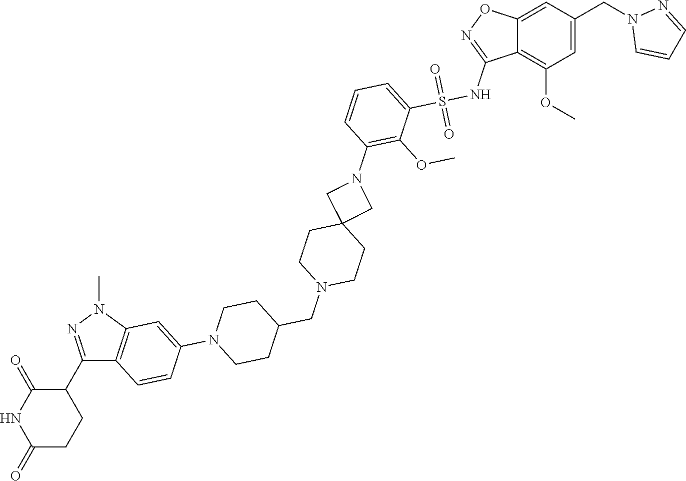 [SMILES: COc1c(N2CC3(CCN(CC4CCN(c5ccc6c(C7CCC(=O)NC7=O)nn(C)c6c5)CC4)CC3)C2)cccc1S(=O)(=O)Nc1noc2cc(Cn3cccn3)cc(OC)c12]

The title compound was synthesized according to procedures analogous to Example 2, using 1-(3-(2,6-dioxopiperidin-3-yl)-1-methyl-1H-indazol-6-yl)piperidine-4-carbaldehyde (which can be prepared as described in WO20240399901A₂). ¹H NMR (400 MHz, DMSO-d₆) δ 10.87 (s, 1H), 10.56 (s, 1H), 7.89 (d, J=2.3 Hz, 1H), 7.58-7.45 (m, 2H), 7.21 (dd, J=8.0, 1.6 Hz, 1H), 7.15 (t, J=7.9 Hz, 1H), 6.91 (d, J=40.6 Hz, 3H), 6.82-6.74 (m, 2H), 6.31 (t, J=2.1 Hz, 1H), 5.46 (s, 2H), 4.27 (dd, J=9.4, 5.1 Hz, 1H), 3.90 (s, 3H), 3.81 (s, 6H), 3.70 (d, J=4.2 Hz, 6H), 3.61 (s, 2H), 3.49 (d, J=12.1 Hz, 2H), 3.12-2.92 (m, 3H), 2.91-2.76 (m, 2H), 2.71-2.57 (m, 2H), 2.36-2.26 (m, 1H), 2.22-1.77 (m, 7H), 1.48-1.29 (m, 2H). LCMS calc. for C₄₅H₅₃N₁₀O₇S [M+H]⁺: m/z=877.4; Found 877.2.

### Example 8. N-(6-((1H-Pyrazol-1-yl)methyl)-4-methoxybenzo[d]isoxazol-3-yl)-3-(7-((1-(6-(2,6-dioxopiperidin-3-yl)-1-methyl-1H-indazol-3-yl)piperidin-4-yl)methyl)-2,7-diazaspiro[3.5]nonan-2-yl)-2-methoxybenzenesulfonamide

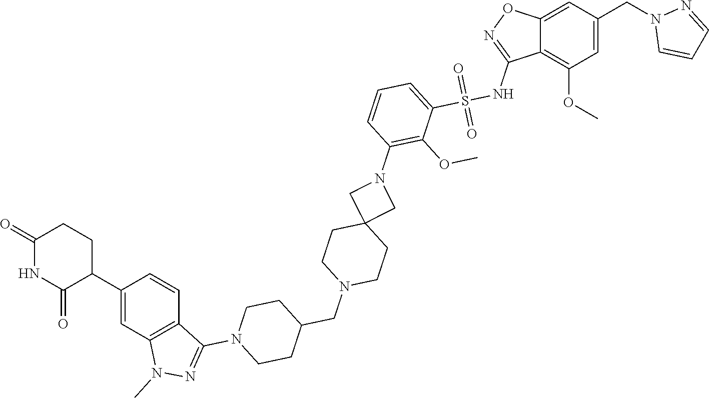 [SMILES: COc1c(N2CC3(CCN(CC4CCN(c5nn(C)c6cc(C7CCC(=O)NC7=O)ccc56)CC4)CC3)C2)cccc1S(=O)(=O)Nc1noc2cc(Cn3cccn3)cc(OC)c12]

### Step 1. 6-Bromo-3-(4-(dimethoxymethyl)piperidin-1-yl)-1-methyl-1H-indazole

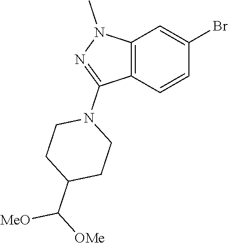 [SMILES: COC(OC)C1CCN(c2nn(C)c3cc(Br)ccc23)CC1]

To a solution of 6-bromo-3-iodo-1-methyl-1H-indazole (4.00 g, 11.9 mmol) and 4-(dimethoxymethyl)piperidine (1.89 g, 11.9 mmol) in DMSO (32 mL) was added potassium carbonate (3.28 g, 23.7 mmol), L-proline (547 mg, 4.75 mmol) and copper(I) iodide (452 mg, 2.37 mmol). The reaction mixture was stirred at 80° C. for 16 h. The reaction was cooled to room temperature, quenched with water, and stirred for 30 minutes. The aqueous phase was extracted with EtOAc (×3). The combined organic layers were dried over Na₂SO₄, filtered, and concentrated. The crude material was purified by silica gel chromatography (0-30% EtOAc/heptanes) to afford the title compound (1.49 g, 4.05 mmol, 34.1%). LCMS calc. for C₁₆H₂₃N₃O₂ [M+H]⁺: m/z=368.1, 370.1; Found 368.4, 370.4.

### Step 2. 6-(2,6-Bis(benzyloxy)pyridin-3-yl)-3-(4-(dimethoxymethyl)piperidin-1-yl)-1-methyl-1H-indazole

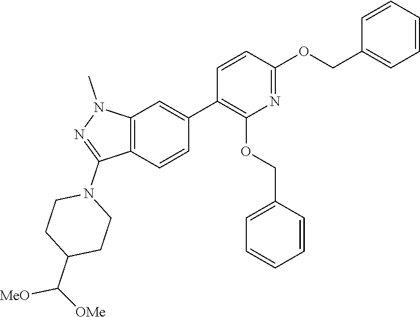 [SMILES: COC(OC)C1CCN(c2nn(C)c3cc(-c4ccc(OCc5ccccc5)nc4OCc4ccccc4)ccc23)CC1]

A solution of 6-bromo-3-(4-(dimethoxymethyl)piperidin-1-yl)-1-methyl-1H-indazole (1.49 g, 4.05 mmol), 2,6-bis(benzyloxy)-3-(4,4,5,5-tetramethyl-1,3,2-dioxa-borolan-2-yl)pyridine (2.53 g, 6.07 mmol), potassium phosphate tribasic (2.58 g, 12.1 mmol), and [1,1′-bis(diphenylphosphino)ferrocene]dichloropalladium(II) dichloromethane (0.331 g, 0.405 mmol) in 1,4-dioxane (30 mL) and water (7.5 mL) was sparged with nitrogen for 10 minutes, then stirred at 100° C. for 4 h. The reaction mixture was cooled to room temperature and poured into water then extracted with EtOAc (3×). The combined organic layers were dried over Na₂SO₄, filtered, and concentrated. The crude material was purified by silica gel chromatography (0-30% EtOAc/heptanes) to afford the title compound (2.20 g, 3.80 mmol, 94.0%). LCMS calc. for C₃₅H₃₉N₄O₄ [M+H]⁺: m/z=579.3; Found 579.6.

### Step 3. 3-(3-(4-(Dimethoxymethyl)piperidin-1-yl)-1-methyl-1H-indazol-6-yl)piperidine-2,6-dione

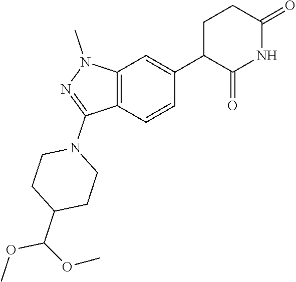 [SMILES: COC(OC)C1CCN(c2nn(C)c3cc(C4CCC(=O)NC4=O)ccc23)CC1]

The title compound was synthesized according to procedures analogous to Example 1, Step 5. LCMS calc. for C₂₁H₂₉N₄O₄ [M+H]⁺: m/z=401.2; Found 401.5.

### Step 4. 1-(6-(2,6-Dioxopiperidin-3-yl)-1-methyl-1H-indazol-3-yl)piperidine-4-carbaldehyde

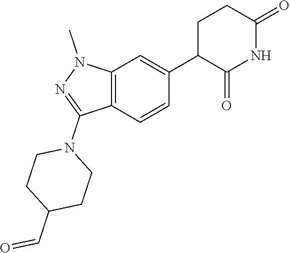 [SMILES: Cn1nc(N2CCC(C=O)CC2)c2ccc(C3CCC(=O)NC3=O)cc21]

The title compound was synthesized according to procedures analogous to Example 4, Step 4. LCMS calc. for C₁₉H₂₃N₄O₃ [M+H]⁺: m/z=355.2; Found 355.1.

### Step 5. N-(6-((1H-Pyrazol-1-yl)methyl)-4-methoxybenzo[d]isoxazol-3-yl)-3-(7-((1-(6-(2,6-dioxopiperidin-3-yl)-1-methyl-1H-indazol-3-yl)piperidin-4-yl)methyl)-2, 7-diazaspiro[3.5]nonan-2-yl)-2-methoxybenzenesulfonamide

The title compound was synthesized according to procedures analogous to Example 2. ¹H NMR (400 MHz, DMSO-d₆) δ 10.88 (s, 1H), 10.55 (s, 1H), 7.89 (d, J=2.3 Hz, 1H), 7.69 (d, J=8.5 Hz, 1H), 7.51 (d, J=1.8 Hz, 1H), 7.32 (s, 1H), 7.21 (dd, J=7.9, 1.6 Hz, 1H), 7.15 (t, J=7.9 Hz, 1H), 6.92-6.82 (m, 2H), 6.83-6.74 (m, 2H), 6.31 (t, J=2.1 Hz, 1H), 5.46 (s, 2H), 3.96 (dd, J=11.8, 4.9 Hz, 1H), 3.89 (d, J=12.3 Hz, 2H), 3.81 (d, J=3.6 Hz, 6H), 3.70 (d, J=4.1 Hz, 6H), 3.61 (s, 2H), 3.49 (d, J=12.1 Hz, 2H), 3.13-2.93 (m, 4H), 2.86 (t, J=12.0 Hz, 2H), 2.72 (ddd, J=17.0, 12.0, 5.1 Hz, 1H), 2.38-2.22 (m, 1H), 2.16-1.76 (m, 8H), 1.51-1.36 (m, 2H). LCMS calc. for C₄₅H₅₃N₁₀O₇S [M+H]⁺: m/z=877.4; Found 877.2.

### Example 9. N-(6-((1H-Pyrazol-1-yl)methyl)-4-methoxybenzo[d]isoxazol-3-yl)-3-(7-((1-(3-(2,6-dioxopiperidin-3-yl)-1-methyl-1H-pyrrolo[2,3-b]pyridin-6-yl)piperidin-4-yl)methyl)-2,7-diazaspiro[3.5]nonan-2-yl)-2-methoxybenzenesulfonamide

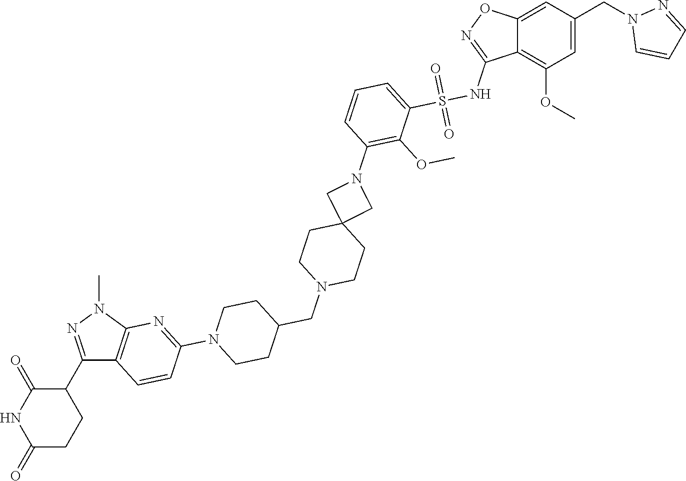 [SMILES: COc1c(N2CC3(CCN(CC4CCN(c5ccc6c(C7CCC(=O)NC7=O)nn(C)c6n5)CC4)CC3)C2)cccc1S(=O)(=O)Nc1noc2cc(Cn3cccn3)cc(OC)c12]

### Step 1. 3-(2,6-Bis(benzyloxy)pyridin-3-yl)-6-chloro-1-methyl-1H-pyrrolo[2,3-b]pyridine

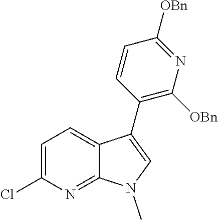 [SMILES: Cn1cc(-c2ccc(OCc3ccccc3)nc2OCc2ccccc2)c2ccc(Cl)nc21]

The title compound was synthesized according to procedures analogous to Example 8, Step 2. LCMS calc. for C₂₇H₂₃ClN₃O₂[M+H]⁺: m/z=456.1; Found 456.4.

### Step 2. 3-(2,6-Bis(benzyloxy)pyridin-3-yl)-6-(4-(dimethoxymethyl)piperidin-1-yl)-1-methyl-1H-pyrrolo[2,3-b]pyridine

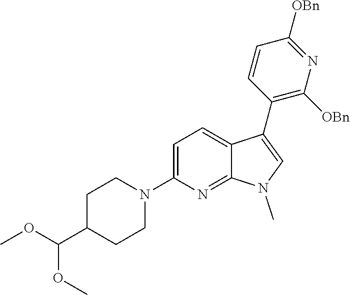 [SMILES: COC(OC)C1CCN(c2ccc3c(-c4ccc(OCc5ccccc5)nc4OCc4ccccc4)cn(C)c3n2)CC1]

The title compound was synthesized according to procedures analogous to Example 4, Step 1. LCMS calc. for C₃₅H₃₉N₄O₄ [M+H]⁺: m/z=579.3; Found 579.7.

### Step 3. 3-(6-(4-(Dimethoxymethyl)piperidin-1-yl)-1-methyl-1H-pyrrolo[2,3-b]pyridin-3-yl)piperidine-2,6-dione

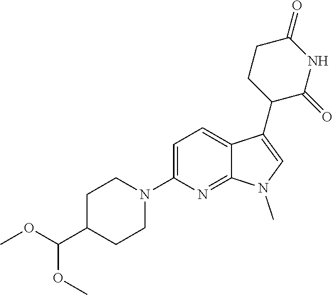 [SMILES: COC(OC)C1CCN(c2ccc3c(C4CCC(=O)NC4=O)cn(C)c3n2)CC1]

The title compound was synthesized according to procedures analogous to Example 1, Step 5. LCMS calc. for C₂₁H₂₉N₄O₄ [M+H]⁺: m/z=401.2; Found 401.5.

### Step 4. 1-(3-(2,6-Dioxopiperidin-3-yl)-1-methyl-1H-pyrrolo[2,3-b]pyridin-6-yl)piperidine-4-carbaldehyde

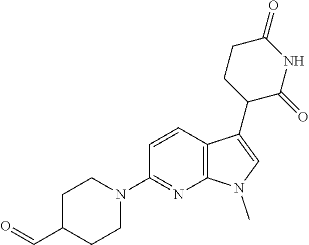 [SMILES: Cn1cc(C2CCC(=O)NC2=O)c2ccc(N3CCC(C=O)CC3)nc21]

The title compound was synthesized according to procedures analogous to Example 4, Step 4. LCMS calc. for C₁₉H₂₃N₄O₃ [M+H]⁺: m/z=355.2; Found 355.1.

### Step 5. N-(6-((1H-Pyrazol-1-yl)methyl)-4-methoxybenzo[d]isoxazol-3-yl)-3-(7-((1-(3-(2,6-dioxopiperidin-3-yl)-1-methyl-1H-pyrrolo[2,3-b]pyridin-6-yl)piperidin-4-yl)methyl)-2, 7-diazaspiro[3.5]nonan-2-yl)-2-methoxybenzenesulfonamide

The title compound was synthesized according to procedures analogous to Example 2. LCMS calc. for C₄₅H₅₃N₁₀O₇S [M+H]⁺: m/z=877.4; Found 877.9.

### Example 10. N-(6-((1H-Pyrazol-1-yl)methyl)-4-methoxybenzo[d]isoxazol-3-yl)-3-(7-((1-(3-(2,4-dioxotetrahydropyrimidin-1(2H)-yl)-1-methyl-1H-indazol-6-yl)piperidin-4-yl)methyl)-2,7-diazaspiro[3.5]nonan-2-yl)-2,6-dimethoxybenzenesulfonamide

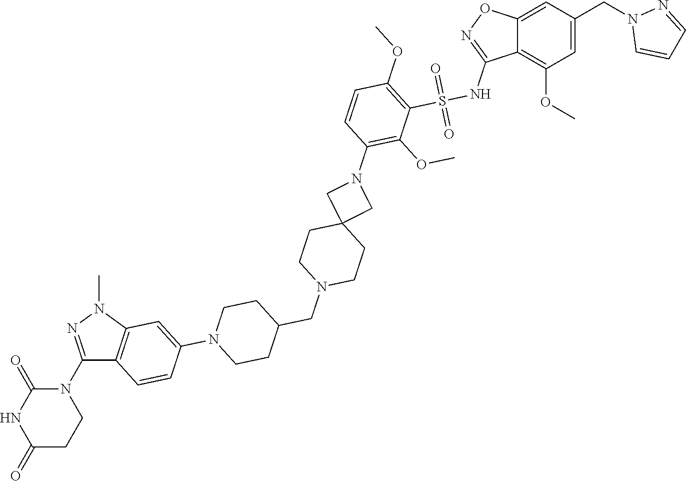 [SMILES: COc1ccc(N2CC3(CCN(CC4CCN(c5ccc6c(N7CCC(=O)NC7=O)nn(C)c6c5)CC4)CC3)C2)c(OC)c1S(=O)(=O)Nc1noc2cc(Cn3cccn3)cc(OC)c12]

The title compound was synthesized according to procedures analogous to Example 2, using N-(6-((1H-pyrazol-1-yl)methyl)-4-methoxybenzo[d]isoxazol-3-yl)-2,6-dimethoxy-3-(2,7-diazaspiro[3.5]nonan-2-yl)benzenesulfonamide (which can be prepared as described in WO2023245150A1) and 1-(3-(2,4-dioxotetrahydropyrimidin-1(2H)-yl)-1-methyl-1H-indazol-6-yl)piperidine-4-carbaldehyde (which can be prepared as described in WO2023249970A1). ¹H NMR (400 MHz, DMSO-d₆) δ 10.52 (s, 1H), 9.60 (s, 1H), 7.89 (d, J=2.2 Hz, 1H), 7.50 (d, J=1.8 Hz, 1H), 7.46 (d, J=9.0 Hz, 1H), 6.98-6.91 (m, 1H), 6.89-6.82 (m, 3H), 6.79 (s, 1H), 6.71 (d, J=9.1 Hz, 1H), 6.31 (t, J=2.1 Hz, 1H), 5.45 (s, 2H), 3.96-3.83 (m, 9H), 3.69 (d, J=3.1 Hz, 7H), 3.64 (s, 2H), 3.55 (s, 2H), 3.48 (d, J=12.3 Hz, 2H), 3.10-2.91 (m, 4H), 2.86-2.71 (m, 4H), 2.15-2.01 (m, 3H), 1.95 (t, J=12.9 Hz, 2H), 1.84 (d, J=12.6 Hz, 2H), 1.37 (q, J=11.4 Hz, 2H). LCMS calc. for C₄₅H₅₄N₁₁O₈S [M+H]⁺: m/z=908.4; Found 908.2.

### Example 11. N-(6-((1H-Pyrazol-1-yl)methyl)-4-methoxybenzo[d]isoxazol-3-yl)-3-(7-((1-(3-(2,6-dioxopiperidin-3-yl)-1-methyl-1H-indazol-6-yl)piperidin-4-yl)methyl)-2,7-diazaspiro[3.5]nonan-2-yl)-2,6-dimethoxybenzenesulfonamide

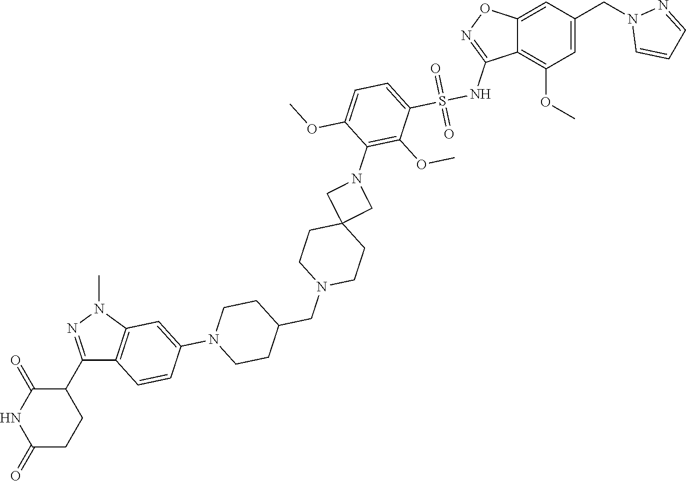 [SMILES: COc1ccc(S(=O)(=O)Nc2noc3cc(Cn4cccn4)cc(OC)c23)c(OC)c1N1CC2(CCN(CC3CCN(c4ccc5c(C6CCC(=O)NC6=O)nn(C)c5c4)CC3)CC2)C1]

The title compound was synthesized according to procedures analogous to Example 2, using 1-(3-(2,6-dioxopiperidin-3-yl)-1-methyl-1H-indazol-6-yl)piperidine-4-carbaldehyde (which can be prepared as described in WO2024039901A₂). LCMS calc. for C₄₆H₅₅N₁₀O₈S [M+H]⁺: m/z=907.4; Found 907.2.

### Example 12. N-(6-((1H-Pyrazol-1-yl)methyl)-4-methoxybenzo[d]isoxazol-3-yl)-4-(7-((1-(1-(2,6-dioxopiperidin-3-yl)-3-methyl-1H-indazol-5-yl)piperidin-4-yl)methyl)-2,7-diazaspiro[3.5]nonan-2-yl)benzenesulfonamide

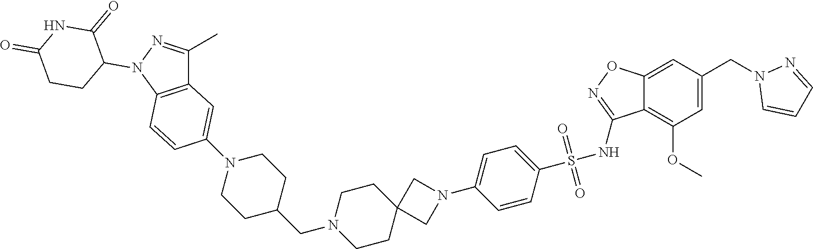 [SMILES: COc1cc(Cn2cccn2)cc2onc(NS(=O)(=O)c3ccc(N4CC5(CCN(CC6CCN(c7ccc8c(c7)c(C)nn8C7CCC(=O)NC7=O)CC6)CC5)C4)cc3)c12]

### Step 1. N-(6-((1H-Pyrazol-1-yl)methyl)-4-methoxybenzo[d]isoxazol-3-yl)-4-bromobenzenesulfonamide

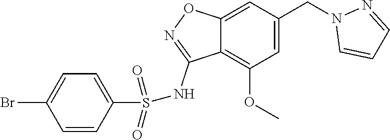 [SMILES: COc1cc(Cn2cccn2)cc2onc(NS(=O)(=O)c3ccc(Br)cc3)c12]

To a solution of 6-((1H-pyrazol-1-yl)methyl)-4-methoxybenzo[d]isoxazol-3-amine (which can be prepared as described in WO2023245150A1) (50.0 mg, 0.205 mmol) and 4-bromobenzenesulfonyl chloride (131 mg, 0.512 mmol) in THF (1.0 mL) was added potassium tert-butoxide (184 mg, 1.64 mmol). The reaction was stirred for 1 h then diluted with 1N HCl (aq) and EtOAc. The layers were separated, and the aqueous layer was extracted with EtOAc (3×). The combined organic layers were washed with brine, dried over MgSO₄, filtered, and concentrated. The crude material was purified by silica gel chromatography (0-10% MeOH/DCM) to afford the title compound (53.4 mg, 0.115 mmol, 56.3%) as a white solid. LCMS calc. for C₁₈H₁₆BrN₄O₄S [M+H]⁺: m/z=463.0, 465.0; Found 462.9, 464.9.

### Step 2. N-(6-((1H-Pyrazol-1-yl)methyl)-4-methoxybenzo[d]isoxazol-3-yl)-4-(2, 7-diazaspiro[3.5]nonan-2-yl)benzenesulfonamide

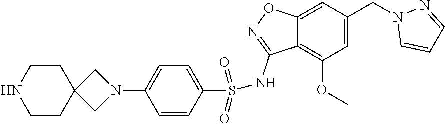 [SMILES: COc1cc(Cn2cccn2)cc2onc(NS(=O)(=O)c3ccc(N4CC5(CCNCC5)C4)cc3)c12]

To a nitrogen-sparged solution of N-(6-((1H-pyrazol-1-yl)methyl)-4-methoxybenzo-[d]isoxazol-3-yl)-4-bromobenzenesulfonamide (53.4 mg, 0.115 mmol), tert-butyl 2,7-diazaspiro[3.5]nonane-7-carboxylate (31.3 mg, 0.138 mmol), and sodium tert-butoxide (55.4 mg, 0.576 mmol) in 1,4-dioxane (1.15 mL) was added BrettPhos Pd G3 (CAS 1470372-59-8, 8.36 mg, 0.00922 mmol). The reaction was stirred for 1 h at 100° C., under a nitrogen atmosphere. The reaction mixture was cooled to room temperature and concentrated. Trifluoroacetic acid (0.5 mL) was added to the residue, and the reaction was stirred for 1 h. The reaction mixture was concentrated then purified by silica gel chromatography (0-25% MeOH/DCM) to afford the title compound (47.0 mg, 0.0920 mmol, 80.0%) as a white solid. C₂₅H₂₉N₆O₄S [M+H]⁺: m/z=509.2; Found 509.0.

### Step 3. N-(6-((1H-Pyrazol-1-yl)methyl)-4-methoxybenzo[d]isoxazol-3-yl)-4-(7-((1-(1-(2,6-dioxopiperidin-3-yl)-3-methyl-1H-indazol-5-yl)piperidin-4-yl)methyl)-2, 7-diazaspiro[3.5]nonan-2-yl)benzenesulfonamide

The title compound was synthesized according to procedures analogous to Example 2, using 1-(1-(2,6-dioxopiperidin-3-yl)-3-methyl-1H-indazol-5-yl)piperidine-4-carbaldehyde (from Example 6, Step 2). LCMS calc. for C₄₄H₅₁N₁₀O₆S [M+H]⁺: m/z=847.4; Found 847.2.

### Example 13. N-(6-((1H-Pyrazol-1-yl)methyl)-4-methoxybenzo[d]isoxazol-3-yl)-4-(7-((1-(2-(2,6-dioxopiperidin-3-yl)-1-oxo-1,2-dihydroisoquinolin-6-yl)piperidin-4-yl)methyl)-2,7-diazaspiro[3.5]nonan-2-yl)benzenesulfonamide

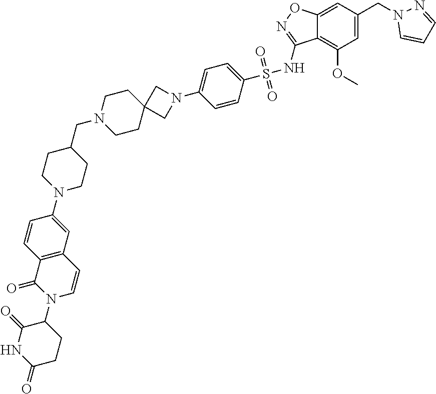 [SMILES: COc1cc(Cn2cccn2)cc2onc(NS(=O)(=O)c3ccc(N4CC5(CCN(CC6CCN(c7ccc8c(=O)n(C9CCC(=O)NC9=O)ccc8c7)CC6)CC5)C4)cc3)c12]

The title compound was synthesized according to procedures analogous to Example 2, using 1-(2-(2,6-dioxopiperidin-3-yl)-1-oxo-1,2-dihydroisoquinolin-6-yl)piperidine-4-carbaldehyde (which can be prepared as described in WO2022099117A1). ¹H NMR (400 MHz, DMSO-d₆) δ 11.01 (s, 1H), 10.42 (s, 1H), 7.99 (d, J=9.0 Hz, 1H), 7.88 (d, J=2.3 Hz, 1H), 7.78 (d, J=8.8 Hz, 2H), 7.50 (d, J=1.8 Hz, 1H), 7.27 (d, J=7.5 Hz, 1H), 7.20 (dd, J=9.3, 2.4 Hz, 1H), 6.98 (d, J=2.4 Hz, 1H), 6.81 (s, 1H), 6.74 (s, 1H), 6.54-6.44 (m, 3H), 6.31 (t, J=2.1 Hz, 1H), 5.44 (s, 2H), 4.00 (d, J=12.7 Hz, 2H), 3.88 (s, 3H), 3.78 (s, 2H), 3.71 (s, 2H), 3.48 (d, J=12.0 Hz, 2H), 3.09-2.77 (m, 7H), 2.63 (t, J=18.0 Hz, 3H), 2.11 (d, J=13.7 Hz, 3H), 2.05-1.90 (m, 3H), 1.82 (d, J=12.6 Hz, 2H), 1.29 (q, J=11.8 Hz, 2H). LCMS calc. for C₄₅H₅₀N₉O₇S [M+H]⁺: m/z=860.4; Found 860.2.

### Example 14. N-(6-((1H-Pyrazol-1-yl)methyl)-4-methoxybenzo[d]isoxazol-3-yl)-4-(7-((1-(1-(2,6-dioxopiperidin-3-yl)-3-methyl-1H-indazol-5-yl)piperidin-4-yl)methyl)-2,7-diazaspiro[3.5]nonan-2-yl)-2-methoxybenzenesulfonamide

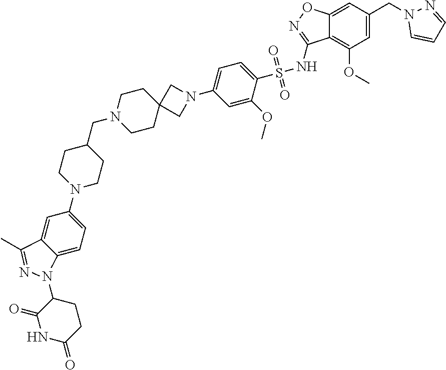 [SMILES: COc1cc(N2CC3(CCN(CC4CCN(c5ccc6c(c5)c(C)nn6C5CCC(=O)NC5=O)CC4)CC3)C2)ccc1S(=O)(=O)Nc1noc2cc(Cn3cccn3)cc(OC)c12]

### Step 1. N-(6-((1H-Pyrazol-1-yl)methyl)-4-methoxybenzo[d]isoxazol-3-yl)-2-methoxy-4-(2, 7-diazaspiro[3.5]nonan-2-yl)benzenesulfonamide

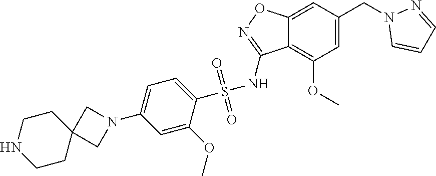 [SMILES: COc1cc(N2CC3(CCNCC3)C2)ccc1S(=O)(=O)Nc1noc2cc(Cn3cccn3)cc(OC)c12]

The title compound was synthesized according to procedures analogous to Example 12, Steps 1-2, using 4-bromo-2-methoxybenzenesulfonyl chloride. LCMS calc. for C₂₆H₃₁N₆O₅S [M+H]⁺: m/z=539.2; Found 539.1.

### Step 2. N-(6-((1H-Pyrazol-1-yl)methyl)-4-methoxybenzo[d]isoxazol-3-yl)-4-(7-((1-(1-(2,6-dioxopiperidin-3-yl)-3-methyl-1H-indazol-5-yl)piperidin-4-yl)methyl)-2, 7-diazaspiro[3.5]nonan-2-yl)-2-methoxybenzenesulfonamide

The title compound was synthesized according to procedures analogous to Example 2, using 1-(1-(2,6-dioxopiperidin-3-yl)-3-methyl-1H-indazol-5-yl)piperidine-4-carbaldehyde (from Example 6, Step 2). LCMS calc. for C₄₅H₅₃N₁₀O₇S [M+H]⁺: m/z=877.4; Found 877.1.

### Example 15. N-(6-((1H-Pyrazol-1-yl)methyl)-4-methoxybenzo[d]isoxazol-3-yl)-4-(7-((1-(1-(2,6-dioxopiperidin-3-yl)-3-methyl-2-oxo-2,3-dihydro-1H-benzo[d]imidazol-5-yl)piperidin-4-yl)methyl)-2,7-diazaspiro[3.5]nonan-2-yl)-2-methoxybenzene-sulfonamide

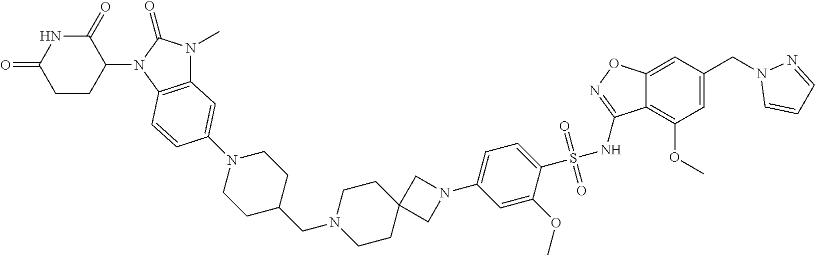 [SMILES: COc1cc(N2CC3(CCN(CC4CCN(c5ccc6c(c5)n(C)c(=O)n6C5CCC(=O)NC5=O)CC4)CC3)C2)ccc1S(=O)(=O)Nc1noc2cc(Cn3cccn3)cc(OC)c12]

The title compound was synthesized according to procedures analogous to Example 2, using 1-(1-(2,6-dioxopiperidin-3-yl)-3-methyl-2-oxo-2,3-dihydro-1H-benzo[d]imidazol-5-yl)piperidine-4-carbaldehyde (which can be prepared as described in WO2022236058A1). LCMS calc. for C₄₅H₅₃N₁₀O₈S [M+H]⁺: m/z=893.4; Found 893.2.

### Example 16. N-(6-((1H-Pyrazol-1-yl)methyl)-4-methoxybenzo[d]isoxazol-3-yl)-4-(4-((1-(1-(2,6-dioxopiperidin-3-yl)-3-methyl-2-oxo-2,3-dihydro-1H-benzo[d]imidazol-5-yl)piperidin-4-yl)methyl)-3,3-dimethylpiperazin-1-yl)benzenesulfonamide

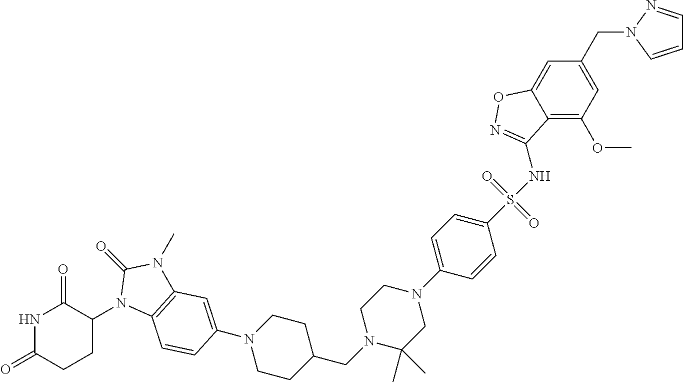 [SMILES: COc1cc(Cn2cccn2)cc2onc(NS(=O)(=O)c3ccc(N4CCN(CC5CCN(c6ccc7c(c6)n(C)c(=O)n7C6CCC(=O)NC6=O)CC5)C(C)(C)C4)cc3)c12]

### Step 1. N-(6-((1H-Pyrazol-1-yl)methyl)-4-methoxybenzo[d]isoxazol-3-yl)-4-(3,3-dimethylpiperazin-1-yl)benzenesulfonamide

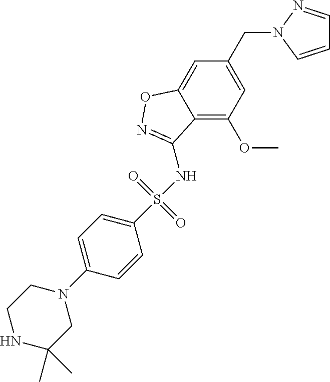 [SMILES: COc1cc(Cn2cccn2)cc2onc(NS(=O)(=O)c3ccc(N4CCNC(C)(C)C4)cc3)c12]

The title compound was synthesized according to procedures analogous to Example 12, Step 2, using tert-butyl 2,2-dimethylpiperazine-1-carboxylate. LCMS calc. for C₂₄H₂₉N₆O₄S [M+H]⁺: m/z=497.2; Found 497.1.

### Step 2. N-(6-((1H-Pyrazol-1-yl)methyl)-4-methoxybenzo[d]isoxazol-3-yl)-4-(4-((1-(1-(2,6-dioxopiperidin-3-yl)-3-methyl-2-oxo-2,3-dihydro-1H-benzo[d]imidazol-5-yl)piperidin-4-yl)methyl)-3,3-dimethylpiperazin-1-yl)benzenesulfonamide

The title compound was synthesized according to procedures analogous to Example 2, using 1-(1-(2,6-dioxopiperidin-3-yl)-3-methyl-2-oxo-2,3-dihydro-1H-benzo[d]imidazol-5-yl)piperidine-4-carbaldehyde (which can be prepared as described in WO2022236058A1). LCMS calc. for C₄₃H₅₁N₁₀O₇S [M+H]⁺: m/z=851.4; Found 851.2.

### Example 17. N-(6-((1H-Pyrazol-1-yl)methyl)-4-methoxybenzo[d]isoxazol-3-yl)-4-((3aS,6aS)-5-((1-(1-(2,6-dioxopiperidin-3-yl)-3-methyl-2-oxo-2,3-dihydro-1H-benzo[d]imidazol-5-yl)piperidin-4-yl)methyl)hexahydropyrrolo[3,4-c]pyrrol-2(1H)-yl)benzenesulfonamide

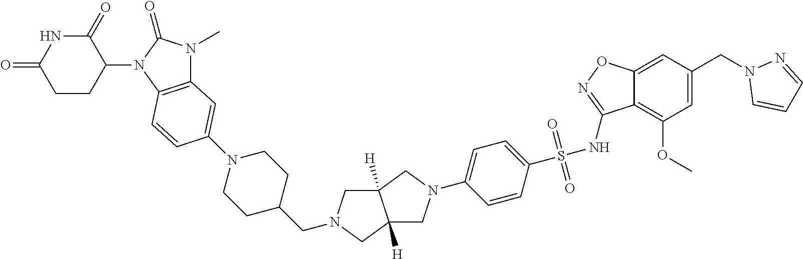 [SMILES: COc1cc(Cn2cccn2)cc2onc(NS(=O)(=O)c3ccc(N4C[C@@H]5CN(CC6CCN(c7ccc8c(c7)n(C)c(=O)n8C7CCC(=O)NC7=O)CC6)C[C@H]5C4)cc3)c12]

### Step 1. N-(6-((1H-Pyrazol-1-yl)methyl)-4-methoxybenzo[d]isoxazol-3-yl)-4-((3aS,6aS)-hexahydropyrrolo[3,4-c]pyrrol-2(1H)-yl)benzenesulfonamide

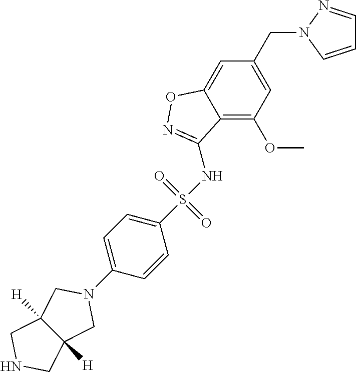 [SMILES: COc1cc(Cn2cccn2)cc2onc(NS(=O)(=O)c3ccc(N4C[C@@H]5CNC[C@H]5C4)cc3)c12]

The title compound was synthesized according to procedures analogous to Example 12, Step 2, using tert-butyl (3aR,6aR)-hexahydropyrrolo[3,4-c]pyrrole-2(1H)-carboxylate (relative stereochemistry, CAS 1818847-81-2). LCMS calc. for C₂₄H₂₇N₆O₄S [M+H]⁺: m/z=495.2; Found 495.0.

### Step 2. N-(6-((1H-Pyrazol-1-yl)methyl)-4-methoxybenzo[d]isoxazol-3-yl)-4-((3aS,6aS)-5-((1-(1-(2,6-dioxopiperidin-3-yl)-3-methyl-2-oxo-2,3-dihydro-1H-benzo[d]imidazol-5-yl)piperidin-4-yl)methyl)hexahydropyrrolo[3,4-c]pyrrol-2(H)-yl)benzenesulfonamide

The title compound was synthesized according to procedures analogous to Example 2, using 1-(1-(2,6-dioxopiperidin-3-yl)-3-methyl-2-oxo-2,3-dihydro-1H-benzo[d]imidazol-5-yl)piperidine-4-carbaldehyde (which can be prepared as described in WO2022236058A1). LCMS calc. for C₄₃H₄₉N₁₀O₇S [M+H]⁺: m/z=849.4; Found 849.2.

### Example 18. N-(6-((1H-Pyrazol-1-yl)methyl)-4-methoxybenzo[d]isoxazol-3-yl)-4-((3aR,6aS)-5-((1-(1-(2,6-dioxopiperidin-3-yl)-3-methyl-2-oxo-2,3-dihydro-1H-benzo[d]imidazol-5-yl)piperidin-4-yl)methyl)hexahydropyrrolo[3,4-c]pyrrol-2(1H)-yl)-2-methoxybenzenesulfonamide

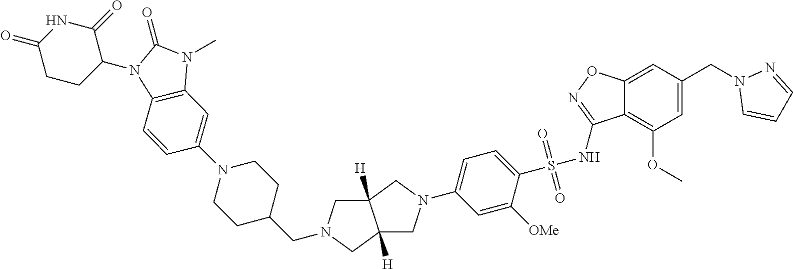 [SMILES: COc1cc(N2C[C@H]3CN(CC4CCN(c5ccc6c(c5)n(C)c(=O)n6C5CCC(=O)NC5=O)CC4)C[C@H]3C2)ccc1S(=O)(=O)Nc1noc2cc(Cn3cccn3)cc(OC)c12]

### Step 1. N-(6-((1H-Pyrazol-1-yl)methyl)-4-methoxybenzo[d]isoxazol-3-yl)-4-((3aR,6aS)-hexahydropyrrolo[3,4-c]pyrrol-2(H)-yl)-2-methoxybenzenesulfonamide

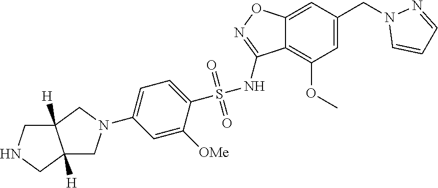 [SMILES: COc1cc(N2C[C@H]3CNC[C@H]3C2)ccc1S(=O)(=O)Nc1noc2cc(Cn3cccn3)cc(OC)c12]

The title compound was synthesized according to procedures analogous to Example 12, Step 2, using tert-butyl (3aR,6aS)-hexahydropyrrolo[3,4-c]pyrrole-2(1H)-carboxylate and N-(6-((1H-pyrazol-1-yl)methyl)-4-methoxybenzo[d]isoxazol-3-yl)-4-bromo-2-methoxybenzenesulfonamide. LCMS calc. for C₂₅H₂₉N₆O₅S [M+H]⁺: m/z=525.2; Found 525.1.

### Step 2. N-(6-((1H-Pyrazol-1-yl)methyl)-4-methoxybenzo[d]isoxazol-3-yl)-4-((3aR,6aS)-5-((1-(1-(2, 6-dioxopiperidin-3-yl)-3-methyl-2-oxo-2,3-dihydro-1H-benzo[d]-imidazol-5-yl)piperidin-4-yl)methyl)hexahydropyrrolo[3,4-c]pyrrol-2(H)-yl)-2-methoxybenzenesulfonamide

The title compound was synthesized according to procedures analogous to Example 2, using 1-(1-(2,6-dioxopiperidin-3-yl)-3-methyl-2-oxo-2,3-dihydro-1H-benzo-[d]imidazol-5-yl)piperidine-4-carbaldehyde (which can be prepared as described in WO2022236058A1). LCMS calc. for C₄₄H₅₁N₁₀O₈S [M+H]⁺: m/z=879.4; Found 879.2.

### Example 19. N-(6-((1H-Pyrazol-1-yl)methyl)-4-methoxybenzo[d]isoxazol-3-yl)-3-(9-((1-(4-(2,6-dioxopiperidin-3-yl)phenyl)piperidin-4-yl)methyl)-3,9-diazaspiro[5.5]-undecan-3-yl)benzenesulfonamide

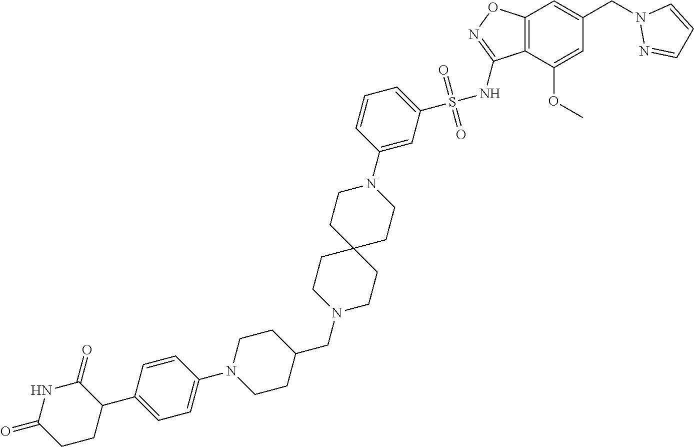 [SMILES: COc1cc(Cn2cccn2)cc2onc(NS(=O)(=O)c3cccc(N4CCC5(CCN(CC6CCN(c7ccc(C8CCC(=O)NC8=O)cc7)CC6)CC5)CC4)c3)c12]

### Step 1. N-(6-((1H-Pyrazol-1-yl)methyl)-4-methoxybenzo[d]isoxazol-3-yl)-3-(3,9-diazaspiro[5.5]undecan-3-yl)benzenesulfonamide

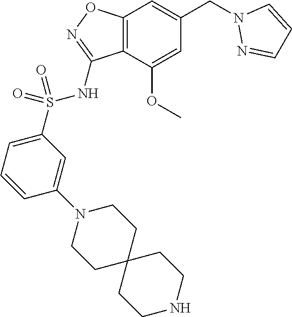 [SMILES: COc1cc(Cn2cccn2)cc2onc(NS(=O)(=O)c3cccc(N4CCC5(CCNCC5)CC4)c3)c12]

The title compound was synthesized according to procedures analogous to Example 12, Step 2 using N-(6-((1H-pyrazol-1-yl)methyl)-4-methoxybenzo[d]isoxazol-3-yl)-3-bromobenzenesulfonamide (which can be prepared as described in WO2023245150A1) and tert-butyl 3,9-diazaspiro[5.5]-undecane-3-carboxylate. LCMS calc. for C₂₇H₃₃N₆O₄S [M+H]⁺: m/z=537.2; Found 537.4.

### Step 2. N-(6-((1H-Pyrazol-1-yl)methyl)-4-methoxybenzo[d]isoxazol-3-yl)-3-(9-((1-(4-(2,6-dioxopiperidin-3-yl)phenyl)piperidin-4-yl)methyl)-3,9-diazaspiro[5.5]undecan-3-yl)benzenesulfonamide

The title compound was synthesized according to procedures analogous to Example 2, using 1-(4-(2,6-dioxopiperidin-3-yl)phenyl)piperidine-4-carbaldehyde (which can be prepared as described in WO2021219070A1). LCMS calc. for C₄₄H₅₃N₈O₆S [M+H]⁺: m/z=821.4; Found 821.7.

### Example 20. N-(6-((1H-Pyrazol-1-yl)methyl)-4-methoxybenzo[d]isoxazol-3-yl)-3-(9-((1-(3-(2,6-dioxopiperidin-3-yl)-1-methyl-1H-indazol-6-yl)piperidin-4-yl)methyl)-3,9-diazaspiro[5.5]undecan-3-yl)benzenesulfonamide

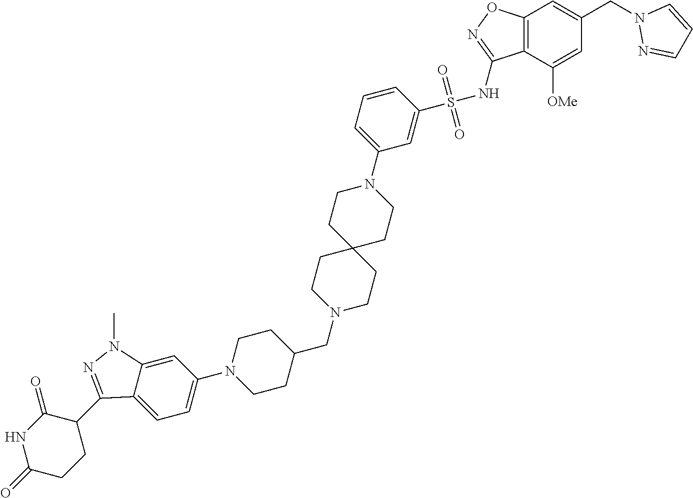 [SMILES: COc1cc(Cn2cccn2)cc2onc(NS(=O)(=O)c3cccc(N4CCC5(CCN(CC6CCN(c7ccc8c(C9CCC(=O)NC9=O)nn(C)c8c7)CC6)CC5)CC4)c3)c12]

The title compound was synthesized according to procedures analogous to Example 2, using N-(6-((1H-pyrazol-1-yl)methyl)-4-methoxybenzo[d]isoxazol-3-yl)-3-(3,9-diazaspiro[5.5]undecan-3-yl)benzenesulfonamide (from Example 19, Step 1) and 1-(3-(2,6-dioxopiperidin-3-yl)-1-methyl-1H-indazol-6-yl)piperidine-4-carbaldehyde (which can be prepared as described in WO2024039901A₂). LCMS calc. for C₄₆H₅₅N₁₀O₆S [M+H]⁺: m/z=875.4; Found 875.8.

### Example 21. N-(6-((1H-Pyrazol-1-yl)methyl)-4-methoxybenzo[d]isoxazol-3-yl)-3-(7-((1-(3-(2,6-dioxopiperidin-3-yl)-1-methyl-1H-indazol-6-yl)piperidin-4-yl)methyl)-2,7-diazaspiro[3.5]nonan-2-yl)-2-(2,2,2-trifluoroethoxy)benzenesulfonamide

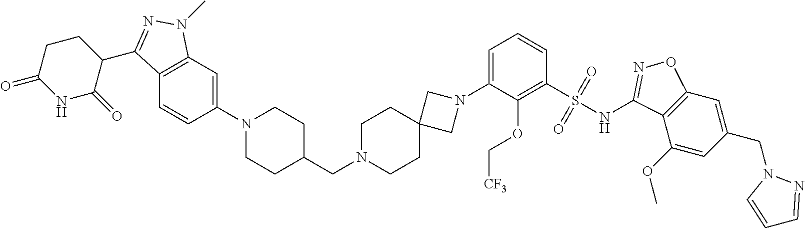 [SMILES: COc1cc(Cn2cccn2)cc2onc(NS(=O)(=O)c3cccc(N4CC5(CCN(CC6CCN(c7ccc8c(C9CCC(=O)NC9=O)nn(C)c8c7)CC6)CC5)C4)c3OCC(F)(F)F)c12]

### Step 1: 1,3-Dibromo-2-(2,2,2-trifluoroethoxy)benzene

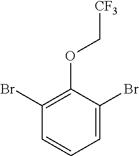 [SMILES: FC(F)(F)COc1c(Br)cccc1Br]

A mixture of 2,6-dibromophenol (1.00 g, 3.97 mmol), N,N-dimethyl formamide (10.0 mL), potassium carbonate (16.50 g, 11.90 mmol), and 1,1,1-trifluoro-2-iodoethane (1.8 mL, 12.00 mmol) were stirred at 100° C. for 5 hours. After cooling to room temperature, the mixture was diluted with water (30 mL) and hexanes (30 mL). The phases were separated, and the aqueous phase was extracted with hexanes (20 mL). The combined organic layers were dried over anhydrous magnesium sulfate, filtered, and concentrated. The crude material was purified by silica gel chromatography (hexanes) to afford the title compound (840 mg, 63% yield) as white crystalline solid. ¹H NMR (400 MHz, CDCl₃) δ 7.56 (d, J=8.1 Hz, 2H), 6.97 (t, J=8.0 Hz, 1H), 4.43 (q, J=8.2 Hz, 2H). ¹⁹F NMR (376 MHz, CDCl₃) δ −74.0.

### Step 2: Benzyl(3-bromo-2-(2,2,2-trifluoroethoxy)phenyl)sulfane

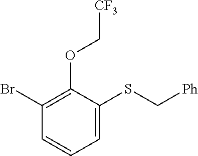 [SMILES: FC(F)(F)COc1c(Br)cccc1SCc1ccccc1]

A mixture of phenylmethanethiol (115 μL, 0.98 mmol), N,N-diisopropylethyl-amine (0.33 mL, 1.90 mmol), 1,3-dibromo-2-(2,2,2-trifluoroethoxy)benzene (313.0 mg, 0.94 mmol), and 1,4-dioxane (8 mL) was sparged with nitrogen for 5 minutes. Tris(di-benzylideneacetone)dipalladium(0) (42.9 mg, 0.05 mmol) and 9,9-dimethyl-4,5-bis(di-phenylphosphino)xanthene (54.2 mg, 0.09 mmol) were added. The mixture was purged with nitrogen for 5 minutes, and then stirred at 100° C. for 4 hours. After cooling to room temperature, the mixture was passed through a pad of silica gel (10 g) eluting with ethyl acetate (50 mL) and concentrated. The crude material was purified by silica gel chromatography (0-15% dichloromethane/hexanes) to afford the title compound (140 mg, 40% yield) as clear oil. ¹H NMR (400 MHz, CDCl₃) δ 7.44 (dd, J=8.1, 1.5 Hz, 1H), 7.34-7.20 (m, 6H), 6.97 (t, J=7.9 Hz, 1H), 4.40 (q, J=8.4 Hz, 2H), 4.15 (s, 2H). ¹⁹F NMR (376 MHz, CDCl₃) δ −74.0.

### Step 3: 3-Bromo-2-(2,2,2-trifluoroethoxy)benzenesulfonyl chloride

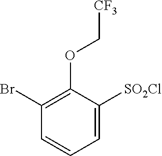 [SMILES: O=S(=O)(Cl)c1cccc(Br)c1OCC(F)(F)F]

To a mixture of benzyl(3-bromo-2-(2,2,2-trifluoroethoxy)phenyl)sulfane (132.0 mg, 0.35 mmol), water (0.5 mL), and acetic acid (2.5 mL) was added 1-chloropyrrolidine-2,5-dione (186.9 mg, 1.4 mmol). The mixture was stirred for 90 min, diluted with ethyl acetate (20 mL), and quenched with brine (10 mL). The phases were separated, and the aqueous phase was extracted with ethyl acetate (10 mL). The combined organic phases were dried over anhydrous magnesium sulfate, filtered, and concentrated. The crude material was purified by silica gel chromatography (0-30% ethyl acetate/hexanes) to afford the title compound (124.0 mg, 100% yield) as colorless oil. ¹H NMR (400 MHz, CDCl₃) δ 8.03-7.98 (m, 2H), 7.34 (t, J=8.1 Hz, 1H), 4.67 (q, J=7.9 Hz, 2H). ¹⁹F NMR (376 MHz, CDCl₃) δ −73.8.

### Step 4: N-(6-((1H-Pyrazol-1-yl)methyl)-4-methoxybenzo[d]isoxazol-3-yl)-3-bromo-2-(2,2,2-trifluoroethoxy)benzenesulfonamide

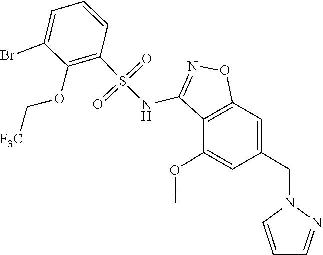 [SMILES: COc1cc(Cn2cccn2)cc2onc(NS(=O)(=O)c3cccc(Br)c3OCC(F)(F)F)c12]

To a mixture of 6-((1H-pyrazol-1-yl)methyl)-4-methoxybenzo[d]isoxazol-3-amine (48.0 mg, 0.20 mmol) and 3-bromo-2-(2,2,2-trifluoroethoxy)benzenesulfonyl chloride (125.1 mg, 0.35 mmol) in tetrahydrofuran (1 mL) was added potassium tert-butoxide (0.69 mL, 0.69 mmol, 1.0 M in tetrahydrofuran) drop wise. The mixture was stirred for 15 minutes. The mixture was next diluted with brine (10 mL) and extracted with ethyl acetate (2×30 mL). The combined organic phases were dried over anhydrous magnesium sulfate, filtered, and concentrated. The crude material was purified by silica gel chromatography (0-5% methanol/dichloromethane) to afford the title compound (99.0 mg, 90% yield) as pale yellow solid. LCMS calc. for C₂₀H₁₇BrF₃N₄O₅S [M+H]⁺: m/z=561.0; Found: 560.7.

### Step 5: tert-Butyl 2-(3-(N-(6-((1H-pyrazol-1-yl)methyl)-4-methoxybenzo-[d]isoxazol-3-yl)sulfamoyl)-2-(2,2,2-trifluoroethoxy)phenyl)-2,7-diazaspiro[3.5]nonane-7-carboxylate

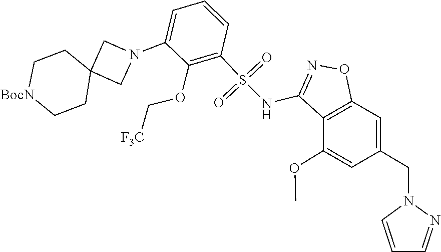 [SMILES: COc1cc(Cn2cccn2)cc2onc(NS(=O)(=O)c3cccc(N4CC5(CCN(C(=O)OC(C)(C)C)CC5)C4)c3OCC(F)(F)F)c12]

A nitrogen sparged mixture of tert-butyl 2-azaspiro[3.5]nonane-7-carboxylate hydrochloride (64.6 mg, 0.25 mmol), sodium tert-butoxide (84.7 mg, 0.88 mmol), N-(6-((1H-pyrazol-1-yl)methyl)-4-methoxybenzo[d]isoxazol-3-yl)-3-bromo-2-(2,2,2-trifluoroethoxy)-benzenesulfonamide (99.0 mg, 0.18 mmol), and BrettPhos Pd G4 (24.4 mg, 0.03 mmol) in 1,4-dioxane (2 mL) was stirred at 100° C. for 1 hour. After cooling to room temperature, the mixture was diluted with dichloromethane, passed through a pad of Celite (10 g), and concentrated. The crude material was purified by silica gel chromatography (0-6% methanol/dichloromethane) to afford the title compound as pale-yellow viscous oil. LCMS calc. for C₃₂H₃₈F₃N₆O₇S [M+H]⁺: m/z=707.2; Found: 707.0.

### Step 6: N-(6-((1H-Pyrazol-1-yl)methyl)-4-methoxybenzo[d]isoxazol-3-yl)-3-(2, 7-diazaspiro[3.5]nonan-2-yl)-2-(2,2,2-trifluoroethoxy)benzenesulfonamide

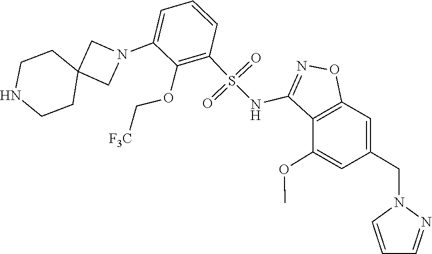 [SMILES: COc1cc(Cn2cccn2)cc2onc(NS(=O)(=O)c3cccc(N4CC5(CCNCC5)C4)c3OCC(F)(F)F)c12]

A mixture of tert-butyl 2-(3-(N-(6-((1H-pyrazol-1-yl)methyl)-4-methoxybenzo-[d]isoxazol-3-yl)sulfamoyl)-2-(2,2,2-trifluoroethoxy)phenyl)-2,7-diazaspiro[3.5]nonane-7-carboxylate (40.0 mg, 0.06 mmol), dichloromethane (1.0 mL), and 2,2,2-trifluoroacetic acid (220 μL, 2.83 mmol) was stirred for 20 minutes. The crude mixture was concentrated, diluted with acetonitrile (5 mL), and purified by preparative (Waters CSH C18, 5 μm particle size, 30×100 mm) HPLC (26.2-46.2% CH₃CN in H₂O with 0.2% TFA in 5 minutes) to afford the title compound as a TFA salt (24.3 mg, 60% yield), a white solid. LCMS calc. for C₂₇H₃₀F₃N₆O₅S [M+H]⁺: m/z=607.2; Found: 607.0.

### Step 7: N-(6-((1H-Pyrazol-1-yl)methyl)-4-methoxybenzo[d]isoxazol-3-yl)-3-(7-((1-(3-(2,6-dioxopiperidin-3-yl)-1-methyl-1H-indazol-6-yl)piperidin-4-yl)methyl)-2, 7-diazaspiro[3.5]nonan-2-yl)-2-(2,2,2-trifluoroethoxy)benzenesulfonamide

To a mixture of 1-(3-(2,6-dioxopiperidin-3-yl)-1-methyl-1H-indazol-6-yl)-piperidine-4-carbaldehyde (7.5 mg, 0.02 mmol) and N-(6-((1H-pyrazol-1-yl)methyl)-4-methoxybenzo[d]isoxazol-3-yl)-3-(2,7-diazaspiro[3.5]nonan-2-yl)-2-(2,2,2-trifluoro-ethoxy)benzenesulfonamide (8.0 mg, 0.01 mmol) in DMSO (1 mL) was added N,N-diisopropylethylamine (13.8 μL, 0.08 mmol) and acetic acid (2.3 μL, 0.04 mmol). The mixture was stirred for 10 minutes. Next, sodium triacetoxyborohydride (14.0 mg, 0.07 mmol) was added and the mixture was stirred for 2 hours. The crude mixture was diluted with DMSO, filtered through a 0.2 um PTFE syringe filter, and purified by preparative (Waters CSH C18, 5 μm particle size, 30×100 mm) HPLC (24.6-44.6% CH₃CN in H₂O with 0.2% TFA in 5 minutes) to afford the title compound as a TFA salt (9.5 mg, 68% yield), a white solid. LCMS calc. for C₄₆H₅₂F₃N₁₀O₇S [M+H]⁺: m/z=945.4; Found: 945.2.

### Example 22. N-(6-((1H-Pyrazol-1-yl)methyl)-4-methoxybenzo[d]isoxazol-3-yl)-2-chloro-3-(7-((1-(3-(2,6-dioxopiperidin-3-yl)-1-methyl-1H-indazol-6-yl)piperidin-4-yl)methyl)-2,7-diazaspiro[3.5]nonan-2-yl)benzenesulfonamide

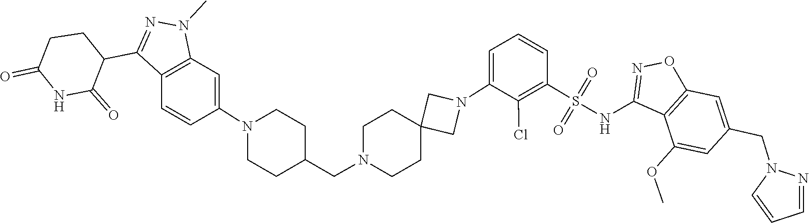 [SMILES: COc1cc(Cn2cccn2)cc2onc(NS(=O)(=O)c3cccc(N4CC5(CCN(CC6CCN(c7ccc8c(C9CCC(=O)NC9=O)nn(C)c8c7)CC6)CC5)C4)c3Cl)c12]

The title compound was synthesized by procedures analogous to that described in Example 21, Steps 2-7. LCMS calc. for C₄₄H₅₀ClN₁₀O₆S [M+H]⁺: m/z=881.3; Found: 881.3.

### Example 23. N-(6-((1H-Pyrazol-1-yl)methyl)-4-methoxybenzo[d]isoxazol-3-yl)-3-(7-((1-(3-(2,6-dioxopiperidin-3-yl)-1-methyl-1H-indazol-6-yl)piperidin-4-yl)methyl)-2,7-diazaspiro[3.5]nonan-2-yl)-5-fluoro-2-methoxybenzenesulfonamide

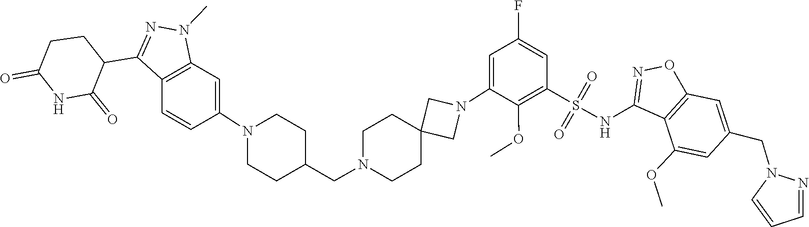 [SMILES: COc1c(N2CC3(CCN(CC4CCN(c5ccc6c(C7CCC(=O)NC7=O)nn(C)c6c5)CC4)CC3)C2)cc(F)cc1S(=O)(=O)Nc1noc2cc(Cn3cccn3)cc(OC)c12]

The title compound was synthesized by procedures analogous to that described in Example 21, Steps 2-7. LCMS calc. for C₄₅H₅₂FN₁₀O₇S [M+H]⁺: m/z=895.4; Found: 895.2.

### Example 24. N-(6-((1H-Pyrazol-1-yl)methyl)-4-methoxybenzo[d]isoxazol-3-yl)-5-chloro-3-(7-((1-(3-(2,6-dioxopiperidin-3-yl)-1-methyl-1H-indazol-6-yl)piperidin-4-yl)methyl)-2,7-diazaspiro[3.5]nonan-2-yl)-2-methoxybenzenesulfonamide

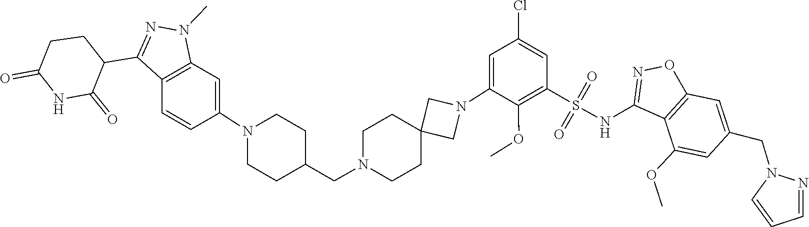 [SMILES: COc1c(N2CC3(CCN(CC4CCN(c5ccc6c(C7CCC(=O)NC7=O)nn(C)c6c5)CC4)CC3)C2)cc(Cl)cc1S(=O)(=O)Nc1noc2cc(Cn3cccn3)cc(OC)c12]

The title compound was synthesized by procedures analogous to that described in Example 21, Step 2-7. LCMS calc. for C₄₅H₅₂ClN₁₀O₇S [M+H]⁺: m/z=911.3; Found: 911.2.

### Example 25. N-(6-((1H-Pyrazol-1-yl)methyl)-4-methoxybenzo[d]isoxazol-3-yl)-3-(7-((1-(3-(2,6-dioxopiperidin-3-yl)-1-methyl-1H-indazol-6-yl)piperidin-4-yl)methyl)-2,7-diazaspiro[3.5]nonan-2-yl)-2-methoxy-5-methylbenzenesulfonamide

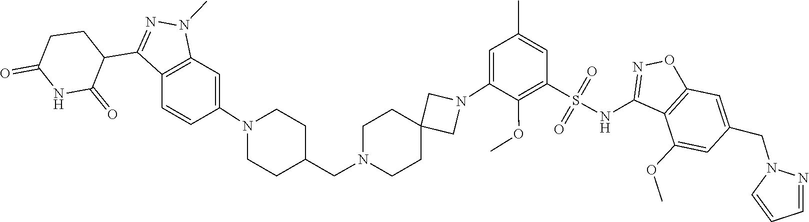 [SMILES: COc1c(N2CC3(CCN(CC4CCN(c5ccc6c(C7CCC(=O)NC7=O)nn(C)c6c5)CC4)CC3)C2)cc(C)cc1S(=O)(=O)Nc1noc2cc(Cn3cccn3)cc(OC)c12]

The title compound was synthesized by procedures analogous to that described in Example 21, Steps 2-7. LCMS calc. for C₄₆H₅₅N₁₀O₇S [M+H]⁺: m/z=891.4; Found: 891.2.

### Example 26. N-(6-((1H-pyrazol-1-yl)methyl)-4-methoxybenzo[d]isoxazol-3-yl)-3-(7-((1-(3-(2,6-dioxopiperidin-3-yl)-1-methyl-1H-indazol-6-yl)piperidin-4-yl)methyl)-2,7-diazaspiro[3.5]nonan-2-yl)-2-(trifluoromethyl)benzenesulfonamide

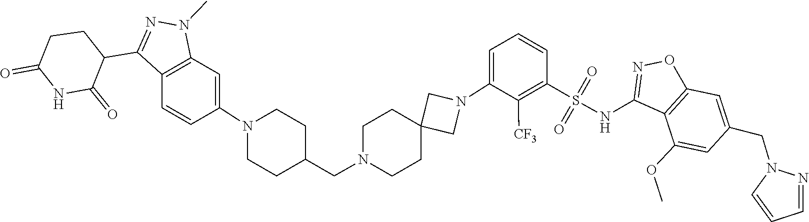 [SMILES: COc1cc(Cn2cccn2)cc2onc(NS(=O)(=O)c3cccc(N4CC5(CCN(CC6CCN(c7ccc8c(C9CCC(=O)NC9=O)nn(C)c8c7)CC6)CC5)C4)c3C(F)(F)F)c12]

The title compound was synthesized by procedures analogous to that described in 21, Seps 2-7. LCMS calc. for C₄₅H₅₀F₃N₁₀O₆S [M+H]⁺: m/z=915.4; Found: 915.2.

### Example 27. N-(6-((1H-pyrazol-1-yl)methyl)-4-methoxybenzo[d]isoxazol-3-yl)-3-(7-((1-(3-(2,6-dioxopiperidin-3-yl)-1-methyl-1H-indazol-6-yl)piperidin-4-yl)methyl)-2,7-diazaspiro[3.5]nonan-2-yl)-2-fluorobenzenesulfonamide

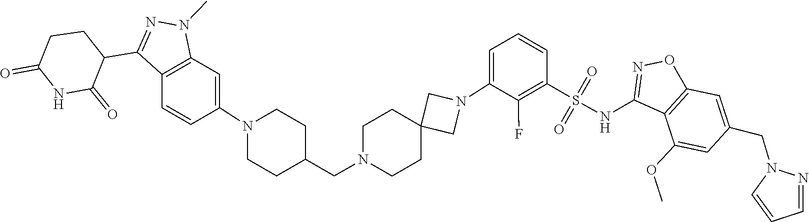 [SMILES: COc1cc(Cn2cccn2)cc2onc(NS(=O)(=O)c3cccc(N4CC5(CCN(CC6CCN(c7ccc8c(C9CCC(=O)NC9=O)nn(C)c8c7)CC6)CC5)C4)c3F)c12]

The title compound was synthesized by procedures analogous to that described in Example 21, Steps 2-7. LCMS calc. for C₄₄H₅₀FN₁₀O₆S [M+H]⁺: m/z=865.4; Found: 865.2.

### Example 28. N-(6-((1H-Pyrazol-1-yl)methyl)-4-methoxybenzo[d]isoxazol-3-yl)-3-(7-((1-(3-(2,6-dioxopiperidin-3-yl)-1-methyl-1H-indazol-6-yl)piperidin-4-yl)methyl)-2,7-diazaspiro[3.5]nonan-2-yl)-5-fluoro-2,6-dimethoxybenzenesulfonamide

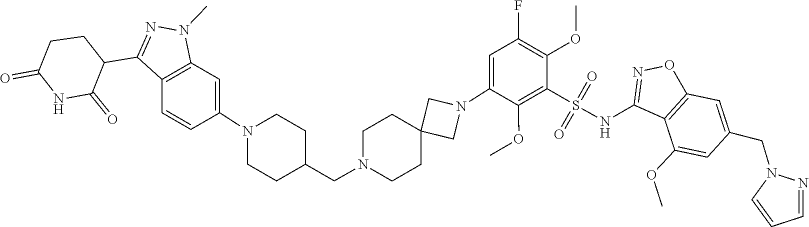 [SMILES: COc1c(F)cc(N2CC3(CCN(CC4CCN(c5ccc6c(C7CCC(=O)NC7=O)nn(C)c6c5)CC4)CC3)C2)c(OC)c1S(=O)(=O)Nc1noc2cc(Cn3cccn3)cc(OC)c12]

### Step 1: Benzyl(3-fluoro-2,6-dimethoxyphenyl)sulfane

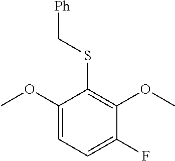

A solution of 1-fluoro-2,4-dimethoxybenzene (570 mg, 3.65 mmol) in THF (20 mL) was purged with nitrogen for 5 minutes at 0° C. The mixture was stirred at 0° C. for 5 minutes then n-butyllithium (1.90 mL, 4.75 mmol, 2.5 M in hexane) was added drop wise. The mixture was stirred at 0° C. for 1 hour. A solution of dibenzyl disulfide (1.03 g, 4.20 mmol) in THF (1 mL) was added drop wise. The ice bath was removed, and the mixture was allowed to warm to room temperature while stirring for 20 min. The reaction mixture was quenched with brine (20 mL) and extracted with ethyl acetate (2×20 mL). The combined organic phases were dried over MgSO₄, filtered, and concentrated. The crude material was purified by silica gel chromatography (0-70% DCM/hexanes) to afford the title compound (0.76 g, 74% yield) as colorless oil. ¹H NMR (400 MHz, CDCl₃) δ 7.27-7.15 (m, 5H), 7.01 (dd, J=10.6, 9.1 Hz, 1H), 6.55 (dd, J=9.1, 3.9 Hz, 1H), 4.07 (s, 2H), 3.84 (s, 3H), 3.71 (s, 3H). ¹⁹F NMR (376 MHz, CDCl₃) δ −138.2

### Step 2: 3-Chloro-5-fluoro-2,6-dimethoxybenzenesulfonyl chloride

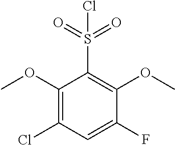

To a solution of benzyl(3-fluoro-2,6-dimethoxyphenyl)sulfane (580 mg, 2.08 mmol) in water (2 mL) and acetic acid (10 mL) was added 1-chloropyrrolidine-2,5-dione (1.11 g, 8.34 mmol). The mixture was stirred at room temperature for 2.5 hours, then diluted with ethyl acetate (20 mL) and brine (10 mL). The phases were separated. The aqueous phase was extracted with ethyl acetate (2×10 mL). The combined organic phases were dried over MgSO₄, filtered, and concentrated. The crude material was purified by silica gel chromatography (0-20% ethyl acetate/hexanes) to afford the title compound (0.46 g, 47% yield) as colorless oil. ¹H NMR (400 MHz, CDCl₃) δ 7.54 (d, J=10.5 Hz, 1H), 4.12 (s, 3H), 4.02 (s, 3H). ¹⁹F NMR (376 MHz, CDCl₃) δ −128.1.

### Step 3: N-(6-((1H-Pyrazol-1-yl)methyl)-4-methoxybenzo[d]isoxazol-3-yl)-3-chloro-5-fluoro-2,6-dimethoxybenzenesulfonamide

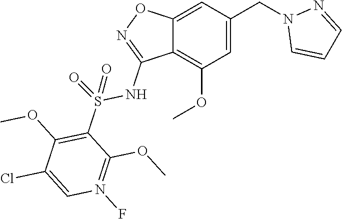

A mixture of 6-((1H-pyrazol-1-yl)methyl)-4-methoxybenzo[d]isoxazol-3-amine (700 mg, 2.87 mmol) (prepared as described in WO2023245150 A1) and 3-chloro-5-fluoro-2,6-dimethoxybenzenesulfonyl chloride (described in Example 39) (2.07 g, 7.16 mmol) in THF (20 mL) was stirred at 0° C. for 10 minutes. Potassium tert-butoxide (1.0 M in THF, 17.2 mL, 17.2 mmol) was added to the mixture drop wise. The mixture continued to stir at 0° C. for 30 min. The reaction was then quenched with brine (5 mL) and extracted with a 6:1 solution of DCM/MeOH (2×20 mL). The combined organic phases were dried over anhydrous MgSO₄, filtered, and concentrated. The crude material was purified by silica gel chromatography (0-6% MeOH/DCM). The resulting solid was triturated with a 4:1 solution of hexanes/EtOAc (40 mL) to afford the title compound (0.85 g, 60% yield) as yellow solid. LCMS calc. for C₂₀H₁₉ClFN₄O₆S [M+H]⁺: m/z=497.1; Found: 497.0.

### Step 4: N-(6-((1H-Pyrazol-1-yl)methyl)-4-methoxybenzo[d]isoxazol-3-yl)-3-fluoro-2,6-dimethoxy-5-(2, 7-diazaspiro[3.5]nonan-2-yl)benzenesulfonamide

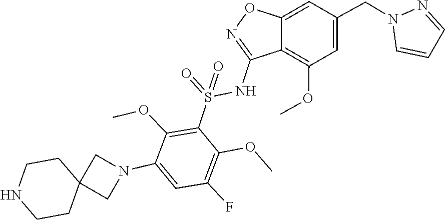 [SMILES: COc1c(F)cc(N2CC3(CCNCC3)C2)c(OC)c1S(=O)(=O)Nc1noc2cc(Cn3cccn3)cc(OC)c12]

A mixture of tert-butyl 2,7-diazaspiro[3.5]nonane-7-carboxylate hydrochloride (135 mg, 0.515 mmol), sodium tert-butoxide (155 mg, 1.61 mmol), N-(6-((1H-pyrazol-1-yl)-methyl)-4-methoxybenzo[d]isoxazol-3-yl)-3-chloro-5-fluoro-2,6-dimethoxybenzenesulfon-amide (160 mg, 0.322 mmol), and dicyclohexyl-[3,6-dimethoxy-2-[2,4,6-tri(propan-2-yl)phenyl]phenyl]phosphane; [2-[2-(methylamino)phenyl]phenyl]-methylsulfonyloxy-palladium (BrettPhos Pd G4) (44 mg, 0.048 mmol), in 1,4-dioxane (4 mL) was sparged with nitrogen for 2 minutes. The reaction mixture was stirred at 90° C. for 1 h. After cooling to room temperature, the mixture was diluted with 20% MeOH/DCM (4 mL), passed through a pad of silica gel eluting with 20% MeOH/DCM (50 mL), and concentrated. The resulting material was diluted with DCM (2 mL), and trifluoroacetic acid (1.21 mL, 16.1 mmol) was added. The mixture was stirred for 20 minutes. The volatiles were then partially removed under reduced pressure. The contents were diluted with MeCN (10 ml) and filtered through a 0.2 um PTFE syringe filter. The crude material was purified by prep-HPLC on a C18 column (23.3-43.3% CH₃CN/0.2% TFA (aq.)) to afford the title compound as a TFA salt (80 mg, 36% yield), a white solid. LCMS calc. for C₂₇H₃₂FN₆O₆S [M+H]⁺: m/z=587.2; Found: 587.1.

### Step 5. N-(6-((1H-Pyrazol-1-yl)methyl)-4-methoxybenzo[d]isoxazol-3-yl)-3-(7-((1-(3-(2,6-dioxopiperidin-3-yl)-1-methyl-1H-indazol-6-yl)piperidin-4-yl)methyl)-2, 7-diazaspiro[3.5]nonan-2-yl)-5-fluoro-2,6-dimethoxybenzenesulfonamide

The title compound was synthesized by procedures analogous to that described in Example 21, Step 7. LCMS calc. for C₄₆H₅₄FN₁₀O₈S [M+H]⁺: m/z=925.4; Found: 925.3.

### Example 29: N-(6-((1H-Pyrazol-1-yl)methyl)-4-methoxybenzo[d]isoxazol-3-yl)-5-(7-((1-(4-(2,6-dioxopiperidin-3-yl)phenyl)piperidin-4-yl)methyl)-2,7-diazaspiro[3.5]-nonan-2-yl)-2-methylbenzenesulfonamide

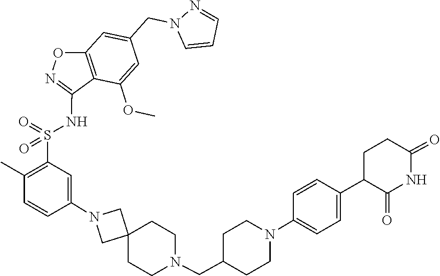 [SMILES: COc1cc(Cn2cccn2)cc2onc(NS(=O)(=O)c3cc(N4CC5(CCN(CC6CCN(c7ccc(C8CCC(=O)NC8=O)cc7)CC6)CC5)C4)ccc3C)c12]

### Step 1: N-(6-((1H-Pyrazol-1-yl)methyl)-4-methoxybenzo[d]isoxazol-3-yl)-5-bromo-2-methylbenzenesulfonamide

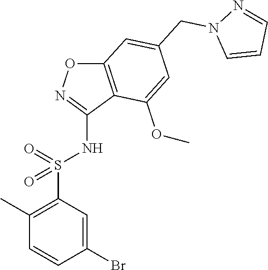 [SMILES: COc1cc(Cn2cccn2)cc2onc(NS(=O)(=O)c3cc(Br)ccc3C)c12]

To a solution of 6-((1H-pyrazol-1-yl)methyl)-4-methoxybenzo[d]isoxazol-3-amine (20 mg, 0.082 mmol) and 5-bromo-2-methylbenzenesulfonyl chloride (55 mg, 0.21 mmol) in THF (1 mL) was added KOtBu (74 mg, 0.66 mmol). The reaction was stirred at room temperature for 1 h then treated with a minimal amount of water and DCM. The mixture was purified directly via SiO₂ FCC (0-100% EtOAc in hexanes) to afford the title compound (17 mg, 44%) as a white solid. LCMS calc. for C₁₉H₁₈BrN₄O₄S [M+H]⁺: m/z=477.0; Found: 476.9.

### Step 2: tert-Butyl 2-(3-(N-(6-((1H-pyrazol-1-yl)methyl)-4-methoxybenzo[d]-isoxazol-3-yl)sulfamoyl)-4-methylphenyl)-2, 7-diazaspiro[3.5]nonane-7-carboxylate

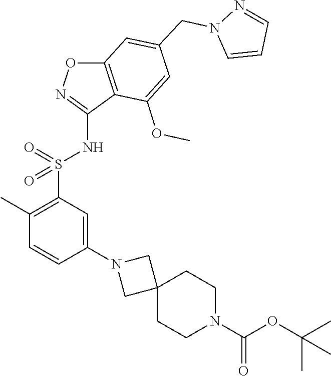 [SMILES: COc1cc(Cn2cccn2)cc2onc(NS(=O)(=O)c3cc(N4CC5(CCN(C(=O)OC(C)(C)C)CC5)C4)ccc3C)c12]

A mixture of N-(6-((1H-pyrazol-1-yl)methyl)-4-methoxybenzo[d]isoxazol-3-yl)-5-bromo-2-methylbenzenesulfonamide (17 mg, 0.036 mmol), tert-butyl 2,7-diazaspiro-[3.5]nonane-7-carboxylate (HCl salt) (11 mg, 0.044 mmol), BrettPhos Pd G3 (3.3 mg, 0.0036 mmol), and NaOtBu (17 mg, 0.18 mmol) in 1,4-dioxane (1 mL) was sparged with N₂ for 1 min. The reaction was stirred at 100° C. for 3.5 h then cooled to room temperature, diluted with MeOH, and purified via prep-HPLC (Waters CSH-C18, 5 μm, 30×100 mm, 53.5-73.5% H₂O (0.2% TFA)/MeCN over 5 min) to afford the title compound (17 mg, 54%) as its TFA salt. LCMS calc. for C₃₁H₃₉N₆O₆S [M+H]⁺: m/z=623.3; Found: 623.1.

### Step 3: tert-Butyl 2-(3-(N-(6-((1H-pyrazol-1-yl)methyl)-4-methoxybenzo[d]-isoxazol-3-yl)sulfamoyl)-4-methylphenyl)-2, 7-diazaspiro[3.5]nonane-7-carboxylate

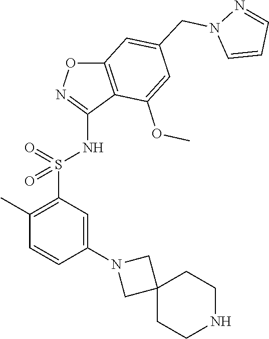 [SMILES: COc1cc(Cn2cccn2)cc2onc(NS(=O)(=O)c3cc(N4CC5(CCNCC5)C4)ccc3C)c12]

To the TFA salt of tert-butyl 2-(3-(N-(6-((1H-pyrazol-1-yl)methyl)-4-methoxy-benzo[d]isoxazol-3-yl)sulfamoyl)-4-methylphenyl)-2,7-diazaspiro[3.5]nonane-7-carboxylate (17 mg, 0.019 mmol) in DCM (1 mL) was added TFA (0.15 mL, 1.9 mmol). The reaction was stirred for 1.5 h then concentrated to afford the TFA salt of the title compound which was used directly in the next step without further purification. LCMS calc. for C₂₆H₃₁N₆O₄S [M+H]⁺: m/z=523.2; Found: 523.0.

### Step 4: N-(6-((1H-Pyrazol-1-yl)methyl)-4-methoxybenzo[d]isoxazol-3-yl)-5-(7-((1-(4-(2,6-dioxopiperidin-3-yl)phenyl)piperidin-4-yl)methyl)-2, 7-diazaspiro[3.5]nonan-2-yl)-2-methylbenzenesulfonamide

To a solution of SO₃-pyridine (12 mg, 0.078 mmol) and Et₃N (22 μL, 0.16 mmol) in DMSO (0.50 mL) was added 3-(4-(4-(hydroxymethyl)piperidin-1-yl)phenyl)piperidine-2,6-dione (7.1 mg, 0.023 mmol). The reaction was stirred at room temperature for 1 h then sequentially treated with AcOH (17 μL, 0.29 mmol), a solution of the TFA salt of N-(6-((1H-pyrazol-1-yl)methyl)-4-methoxybenzo[d]isoxazol-3-yl)-2-methyl-5-(2,7-diazaspiro-[3.5]nonan-2-yl)benzenesulfonamide, and NaBH(OAc)₃ (12 mg, 0.058 mmol). The reaction was stirred at room temperature for 100 min then diluted with MeOH and purified via prep-HPLC (Waters CSH Fluoro-Phenyl, 5 μm, 30×100 mm, 21.4-41.4% H₂O (0.2% TFA)/MeCN over 5 min) to afford the title compound (4.7 mg, 23% over 2 steps) as a white solid. LCMS calc. for C₄₃H₅₁N₈O₆S [M+H]⁺: m/z=807.4; Found: 807.1.

### Example 30: N-(6-((1H-Pyrazol-1-yl)methyl)-4-methoxybenzo[d]isoxazol-3-yl)-3-(7-((1-(4-(2,6-dioxopiperidin-3-yl)phenyl)piperidin-4-yl)methyl)-2,7-diazaspiro[3.5]-nonan-2-yl)-2-methylbenzenesulfonamide

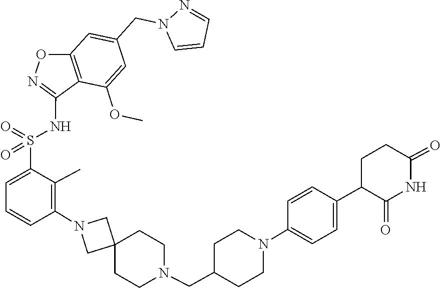 [SMILES: COc1cc(Cn2cccn2)cc2onc(NS(=O)(=O)c3cccc(N4CC5(CCN(CC6CCN(c7ccc(C8CCC(=O)NC8=O)cc7)CC6)CC5)C4)c3C)c12]

### Step 1: N-(6-((1H-Pyrazol-1-yl)methyl)-4-methoxybenzo[d]isoxazol-3-yl)-2-methyl-3-(2, 7-diazaspiro[3.5]nonan-2-yl)benzenesulfonamide

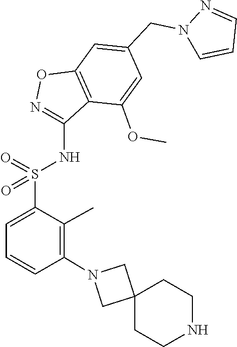 [SMILES: COc1cc(Cn2cccn2)cc2onc(NS(=O)(=O)c3cccc(N4CC5(CCNCC5)C4)c3C)c12]

The TFA salt of the title compound was prepared according to procedures analogous to Example 29, Steps 1-3, using the appropriate starting materials and intermediates. LCMS calc. for C₂₆H₃₁N₆O₄S [M+H]⁺: m/z=523.2; Found: 523.1.

### Step 2: N-(6-((1H-Pyrazol-1-yl)methyl)-4-methoxybenzo[d]isoxazol-3-yl)-3-(7-((1-(4-(2,6-dioxopiperidin-3-yl)phenyl)piperidin-4-yl)methyl)-2, 7-diazaspiro[3.5]nonan-2-yl)-2-methylbenzenesulfonamide

To a solution of SO₃-pyridine (23 mg, 0.15 mmol) and Et₃N (41 μL, 0.29 mmol) in DMSO (0.5 mL) was added 3-(4-(4-(hydroxymethyl)piperidin-1-yl)phenyl)piperidine-2,6-dione (which can be prepared as described in WO2021219070A1) (15 mg, 0.049 mmol). The reaction was stirred at room temperature for 80 min then added to a solution of N-(6-((1H-pyrazol-1-yl)methyl)-4-methoxybenzo[d]isoxazol-3-yl)-2-methyl-3-(2,7-diazaspiro[3.5]nonan-2-yl)benzene-sulfonamide (TFA salt) (18 mg, 0.024 mmol) and AcOH (35 μL, 0.61 mmol) in DMF (1 mL). The resulting solution was treated with NaBH(OAc)₃ (17 mg, 0.078 mmol), stirred for 1 h, then diluted with MeOH and purified via prep-HPLC (Waters CSH Phenyl-Hexyl, 5 μm, 30×100 mm, 17.3-37.3% H₂O (0.2% TFA)/MeCN over 5 min) to give the TFA salt of the title compound (14 mg, 55%) as a white solid. LCMS calc. for C₄₃H₅₁N₈O₆S [M+H]⁺: m/z=807.4; Found: 807.2.

### Example 31: N-(6-((1H-Pyrazol-1-yl)methyl)-4-methoxybenzo[d]isoxazol-3-yl)-4-(7-((1-(3-(2,6-dioxopiperidin-3-yl)-1-methyl-1H-indazol-6-yl)piperidin-4-yl)methyl)-2,7-diazaspiro[3.5]nonan-2-yl)-2-methylbenzenesulfonamide

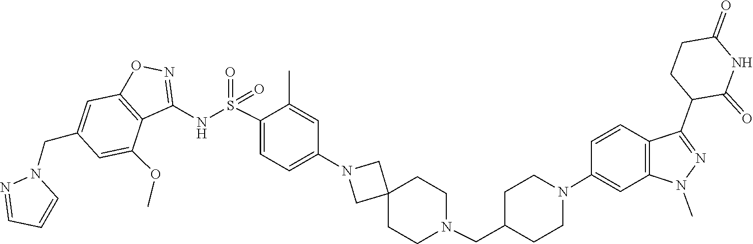 [SMILES: COc1cc(Cn2cccn2)cc2onc(NS(=O)(=O)c3ccc(N4CC5(CCN(CC6CCN(c7ccc8c(C9CCC(=O)NC9=O)nn(C)c8c7)CC6)CC5)C4)cc3C)c12]

### Step 1: N-(6-((1H-Pyrazol-1-yl)methyl)-4-methoxybenzo[d]isoxazol-3-yl)-2-methyl-4-(2, 7-diazaspiro[3.5]nonan-2-yl)benzenesulfonamide

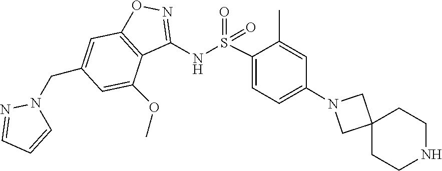 [SMILES: COc1cc(Cn2cccn2)cc2onc(NS(=O)(=O)c3ccc(N4CC5(CCNCC5)C4)cc3C)c12]

The TFA salt of the title compound was prepared according to procedures analogous to Example 29, Steps 1-3, using the appropriate starting materials and intermediates. LCMS calc. for C₂₆H₃₁N₆O₄S [M+H]⁺: m/z=523.2; Found: 523.1.

### Step 2: N-(6-((1H-Pyrazol-1-yl)methyl)-4-methoxybenzo[d]isoxazol-3-yl)-4-(7-((1-(3-(2,6-dioxopiperidin-3-yl)-1-methyl-1H-indazol-6-yl)piperidin-4-yl)methyl)-2, 7-diaza-spiro[3.5]nonan-2-yl)-2-methylbenzenesulfonamide

To a solution of 1-(3-(2,6-dioxopiperidin-3-yl)-1-methyl-1H-indazol-6-yl)-piperidine-4-carbaldehyde (7.70 mg, 0.0217 mmol) (which can be prepared as described in WO2024039901A₂) and the TFA salt of N-(6-((1H-pyrazol-1-yl)methyl)-4-methoxybenzo-[d]isoxazol-3-yl)-2-methyl-4-(2,7-diazaspiro[3.5]nonan-2-yl)benzenesulfonamide (13.6 mg, 0.0181 mmol) in DMF (1 mL) was added AcOH (5.18 μL, 0.0905 mmol), followed by NaBH(OAc)₃ (57.6 mg, 0.272 mmol). The reaction was stirred at room temperature for 45 min then diluted with MeOH and purified via prep-HPLC (Waters CSH Phenyl-Hexyl, 5 μm, 30×100 mm, 11.7-29.7% H₂O (0.2% o TFA)/MeCN over 12 min) to afford the TFA salt of the title compound (8.40 mg, 42.60%) as a white solid. LCMS calc. for C₄₅H₅₃N₁O₆S [M+H]: m/z=861.4; Found: 861.3.

### Examples 32 and 33

Examples 32 and 33 are set forth in Table 1 and were prepared using procedures analogous to Example 29, Steps 1-2, using the appropriate intermediates, as well as commercial starting materials. The following compounds are TFA salts unless otherwise noted.

TABLE 1
Examples 32 and 33

| | | [M + H]⁺: |
|---|---|---|
| Ex | Structure/Name | m/z found |
| 32 | 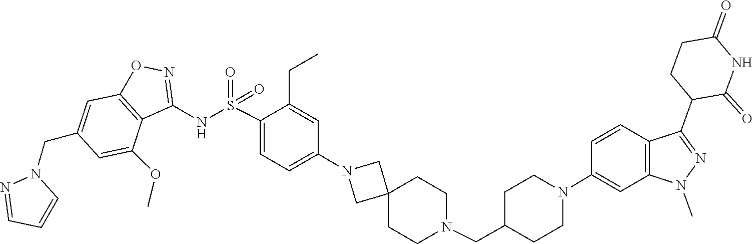 [SMILES: CCc1cc(N2CC3(CCN(CC4CCN(c5ccc6c(C7CCC(=O)NC7=O)nn(C)c6c5)CC4)CC3)C2)ccc1S(=O)(=O)Nc1noc2cc(Cn3cccn3)cc(OC)c12] | 875.3 |
| | N-(6-((1H-Pyrazol-1-yl)methyl)-4-methoxybenzo[d]isoxazol-3-yl)-4-(7- | |
| | ((1-(3-(2,6-dioxopiperidin-3-yl)-1-methyl-1H-indazol-6-yl)piperidin-4- | |
| | yl)methyl)-2,7-diazaspiro[3.5]nonan-2-yl)-2-ethylbenzenesulfonamide | |
| 33 | 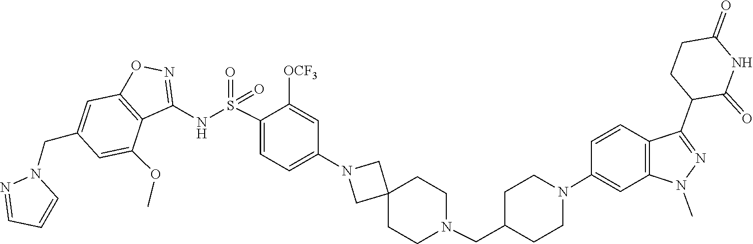 [SMILES: COc1cc(Cn2cccn2)cc2onc(NS(=O)(=O)c3ccc(N4CC5(CCN(CC6CCN(c7ccc8c(C9CCC(=O)NC9=O)nn(C)c8c7)CC6)CC5)C4)cc3OC(F)(F)F)c12] | 931.2 |
| | N-(6-((1H-Pyrazol-1-yl)methyl)-4-methoxybenzo[d]isoxazol-3-yl)-4-(7- | |
| | ((1-(3-(2,6-dioxopiperidin-3-yl)-1-methyl-1H-indazol-6-yl)piperidin- | |
| | 4-yl)methyl)-2,7-diazaspiro[3.5]nonan-2-yl)-2-(trifluoromethoxy)- | |
| | benzenesulfonamide | |

### Example 34: N-(6-((1H-Pyrazol-1-yl)methyl)-4-methoxybenzo[d]isoxazol-3-yl)-3-(7-((1-(3-(2,6-dioxopiperidin-3-yl)-1-methyl-1H-indazol-6-yl)piperidin-4-yl)methyl)-7-azaspiro[3.5]nonan-2-yl)benzenesulfonamide

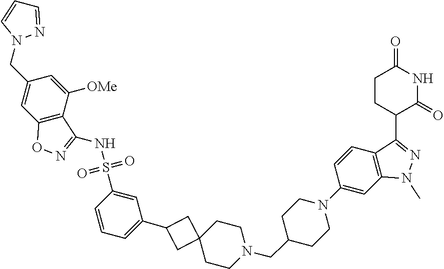 [SMILES: COc1cc(Cn2cccn2)cc2onc(NS(=O)(=O)c3cccc(C4CC5(CCN(CC6CCN(c7ccc8c(C9CCC(=O)NC9=O)nn(C)c8c7)CC6)CC5)C4)c3)c12]

### Step 1. (7-(tert-Butoxycarbonyl)-7-azaspiro[3.5]nonan-2-yl)zinc(II) iodide

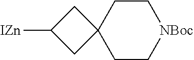

To a dried mixture of zinc (335 mg, 5.12 mmol) and lithium chloride (217 mg, 5.12 mmol) under nitrogen was added THF (1.7 mL), chlorotrimethylsilane (40.0 mL, 0.340 mmol) and 1,2-dibromoethane (30.0 mL, 0.340 mmol) were added, sequentially. The mixture was heated to 45° C. until bubbling began then stirred at the same temperature for an additional 10 min. The reaction was cooled to rt, and a solution of tert-butyl 2-iodo-7-azaspiro[3.5]nonane-7-carboxylate (1.20 g, 3.42 mmol) in THF (1.7 mL) was added under nitrogen and the mixture was stirred at 45° C. overnight. The mixture was filtered through a 0.2 mm PTFE syringe filter to afford a clear, 0.6 M solution of the title compound in THF. The solution was stored under nitrogen.

### Step 2. tert-Butyl 2-(3-(N-(6-((1H-pyrazol-1-yl)methyl)-4-methoxybenzo[d]-isoxazol-3-yl)sulfamoyl)phenyl)-7-azaspiro[3.5]nonane-7-carboxylate

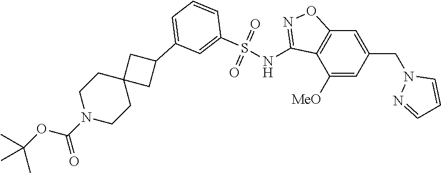 [SMILES: COc1cc(Cn2cccn2)cc2onc(NS(=O)(=O)c3cccc(C4CC5(CCN(C(=O)OC(C)(C)C)CC5)C4)c3)c12]

To a solution of N-(6-((1H-pyrazol-1-yl)methyl)-4-methoxybenzo[d]isoxazol-3-yl)-3-bromobenzenesulfonamide (0.10 g, 0.22 mmol) (which can be prepared as described in WO2023245150A1) in THF (2.2 mL) was added SPhos Pd G2 (CAS 1375325-64-6) (16 mg, 0.02 mmol). The reaction was sparged with nitrogen for 2 minutes then treated with (7-(tert-butoxycarbonyl)-7-azaspiro-[3.5]nonan-2-yl)zinc(II) iodide (0.6 M in THF, 1.44 mL, 0.86 mmol). The reaction was stirred at 60° C. for 1 hr then cooled to rt and directly purified by silica gel chromatography (0-5% MeOH/DCM) to give the title compound (88 mg, 67%) as a white solid. LCMS calcd for C₃₁H₃₈N₅O₆S [M+H]⁺: m/z=608.3; Found: 608.2.

### Step 3. N-(6-((1H-Pyrazol-1-yl)methyl)-4-methoxybenzo[d]isoxazol-3-yl)-3-(7-azaspiro[3.5]nonan-2-yl)benzenesulfonamide

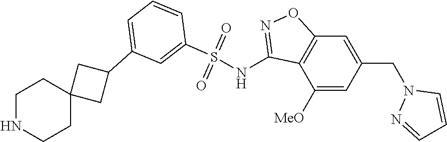 [SMILES: COc1cc(Cn2cccn2)cc2onc(NS(=O)(=O)c3cccc(C4CC5(CCNCC5)C4)c3)c12]

To a solution of tert-butyl 2-(3-(N-(6-((1H-pyrazol-1-yl)methyl)-4-methoxybenzo-[d]isoxazol-3-yl)sulfamoyl)phenyl)-7-azaspiro[3.5]nonane-7-carboxylate (88 mg, 0.14 mmol) in DCM (1.4 mL) was added 2,2,2-trifluoroacetic acid (0.44 mL, 5.8 mmol). The reaction stirred at rt for 1 hr then concentrated under pressure to give the TFA salt of the title compound (70 mg, 95%) as viscous brown oil, which was used in the next step without further purification. LCMS calcd for C₂₆H₃₀N₅O₄S [M+H]⁺: m/z=508.2; Found: 508.1.

### Step 4. N-(6-((1H-Pyrazol-1-yl)methyl)-4-methoxybenzo[d]isoxazol-3-yl)-3-(7-((1-(3-(2,6-dioxopiperidin-3-yl)-1-methyl-1H-indazol-6-yl)piperidin-4-yl)methyl)-7-azaspiro-[3.5]nonan-2-yl)benzenesulfonamide

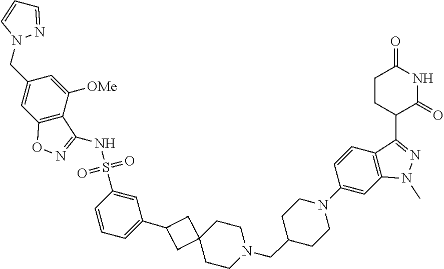 [SMILES: COc1cc(Cn2cccn2)cc2onc(NS(=O)(=O)c3cccc(C4CC5(CCN(CC6CCN(c7ccc8c(C9CCC(=O)NC9=O)nn(C)c8c7)CC6)CC5)C4)c3)c12]

To 1-[3-(2,6-dioxopiperidin-3-yl)-1-methylindazol-6-yl]piperidine-4-carbaldehyde (14 mg, 0.040 mmol) (which can be prepared as described in WO2024039901A₂) and the TFA salt of N-(6-((1H-pyrazol-1-yl)-methyl)-4-methoxybenzo[d]isoxazol-3-yl)-3-(7-azaspiro[3.5]nonan-2-yl)benzene-sulfonamide (10 mg, 0.020 mmol) in DMSO (1.1 mL) was added N,N-diisopropylethyl-amine (0.034 mL, 0.20 mmol). The reaction was stirred for 10 min at rt then treated with sodium triacetoxyborohydride (21 mg, 0.099 mmol) and acetic acid (0.0056 mL, 0.099 mmol). The reaction was stirred for 1 hr at rt then diluted with DMSO, filtered, and purified by prep-HPLC (Waters CSH-C18, 5 μm, 30×100 mm, 20.4-40.4% CH₃CN in H₂O with 0.2% TFA over 5 minutes) to yield the title compound (6.8 mg, 36%) as a white TFA salt. LCMS calcd for C₄₅H₅₂N₉O₆S [M+H]⁺: m/z=846.4; Found: 846.3.

### Example 35: N-(6-((1H-Pyrazol-1-yl)methyl)-4-methoxybenzo[d]isoxazol-3-yl)-3-(2-((4-(3-(2,4-dioxotetrahydropyrimidin-1(2H)-yl)-1-methyl-1H-indazol-6-yl)piperidin-1-yl)methyl)-7-azaspiro[3.5]nonan-7-yl)benzenesulfonamide

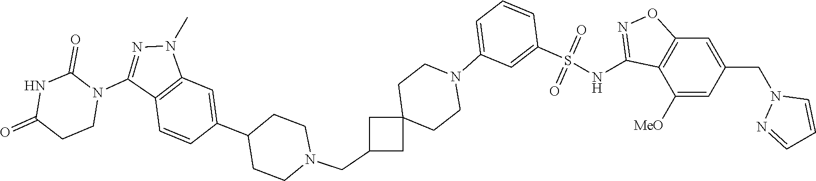 [SMILES: COc1cc(Cn2cccn2)cc2onc(NS(=O)(=O)c3cccc(N4CCC5(CC4)CC(CN4CCC(c6ccc7c(N8CCC(=O)NC8=O)nn(C)c7c6)CC4)C5)c3)c12]

### Step 1. tert-Butyl 2-(((tert-butyldiphenylsilyl)oxy)methyl)-7-azaspiro[3.5]nonane-7-carboxylate

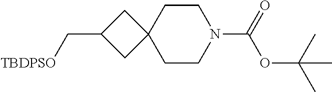

To a solution of tert-butyl 2-(hydroxymethyl)-7-azaspiro[3.5]nonane-7-carboxylate (2.00 mg, 7.83 mmol) and imidazole (640 mg, 9.40 mmol) in DCM (65 mL) was added tert-butylchlorodiphenylsilane (2.44 mL, 9.40 mmol), dropwise. The reaction mixture was stirred at rt overnight then washed with 1N HCl (aq) (100 mL). The organic layer was washed with brine (100 mL) and the combined organic layers were dried over anhydrous sodium sulfate then concentrated. The resulting residue was purified by silica gel chromatography (0-15% EtOAc/hexanes) to give the title compound (3.80 g, 98.3%) as a colorless, viscous oil. LCMS calcd for C₃₀H₄₄NO₃Si [M+H]⁺: m/z=494.3; Found: 494.0.

### Step 2. 2-(((tert-Butyldiphenylsilyl)oxy)methyl)-7-azaspiro[3.5]nonane

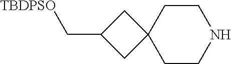

To a solution of tert-butyl 2-(((tert-butyldiphenylsilyl)oxy)methyl)-7-azaspiro-[3.5]nonane-7-carboxylate (3.80 mg, 7.7 mmol) in DCM (38 mL) was added 2,2,2-trifluoroacetic acid (14.7 mL, 192 mmol) and stirred at rt overnight. The reaction was washed with sat. NaHCO₃ (aq) (3×100 mL) and brine (100 mL). The combined organic layers were dried over anhydrous sodium sulfate then concentrated under reduced pressure to give a crude residue which was purified by silica gel chromatography (0-10% MeOH/DCM with 2% triethylamine) to give the title compound (1.70 mg, 56.1%) as a colorless, viscous oil. LCMS calcd for C₂₅H₃₆NOSi [M+H]⁺: m/z=394.3; Found: 394.2.

### Step 3. N-(6-((1H-Pyrazol-1-yl)methyl)-4-methoxybenzo[d]isoxazol-3-yl)-3-(2-(((tert-butyldiphenylsilyl)oxy)methyl)-7-azaspiro[3.5]nonan-7-yl)benzenesulfonamide

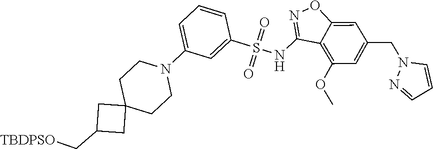

A mixture of 2-(((tert-butyldiphenylsilyl)oxy)methyl)-7-azaspiro[3.5]nonane (476 mg, 1.21 mmol), N-(6-((1H-pyrazol-1-yl)methyl)-4-methoxybenzo[d]isoxazol-3-yl)-3-bromobenzenesulfonamide (0.400 g, 0.86 mmol) (which can be prepared as described in WO2023245150A1), sodium tert-butoxide (249 mg, 2.59 mmol) and BrettPhos Pd G4 (CAS 1599466-83-7) (79.4 mg, 0.0863 mmol) in 1,4-dioxane (8.6 mL) was degassed and then stirred at 90° C. for 1.5 hrs. The solvent was removed under reduced pressure and the resulting residue was purified by silica gel chromatography (0-5% MeOH/DCM to afford the title compound (317 mg, 42.6%) as pale yellow solid. LCMS calcd for C₄₃H₅₀N₅O₅SSi [M+H]⁺: m/z=776.3; Found: 776.1.

### Step 3. N-(6-((1H-Pyrazol-1-yl)methyl)-4-methoxybenzo[d]isoxazol-3-yl)-3-(2-(hydroxymethyl)-7-azaspiro[3.5]nonan-7-yl)benzenesulfonamide

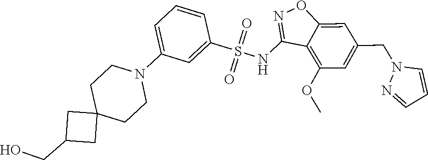 [SMILES: COc1cc(Cn2cccn2)cc2onc(NS(=O)(=O)c3cccc(N4CCC5(CC4)CC(CO)C5)c3)c12]

To a solution of N-(6-((1H-pyrazol-1-yl)methyl)-4-methoxybenzo[d]isoxazol-3-yl)-3-(2-(((tert-butyldiphenylsilyl)oxy)methyl)-7-azaspiro[3.5]nonan-7-yl)benzene-sulfonamide (317 mg, 0.408 mmol) in THF (4.0 mL) was added tetrabutylammonium fluoride (1 M in THF) (1.63 mL, 1.63 mmol). The reaction mixture was stirred at rt for 2 h, diluted with DCM (20 mL), and washed with brine (2×20 mL). The combined organic layers were dried over anhydrous sodium sulfate, concentrated, then purified by silica gel chromatography eluting (0-5% MeOH/DCM) to give the title compound (199 mg, 90.6% yield) as a colorless, viscous oil. LCMS calcd for C₂₇H₃₂N₅O₅S [M+H]⁺: m/z=538.2; Found: 538.0.

### Step 4. N-(6-((1H-Pyrazol-1-yl)methyl)-4-methoxybenzo[d]isoxazol-3-yl)-3-(2-formyl-7-azaspiro[3.5]nonan-7-yl)benzenesulfonamide

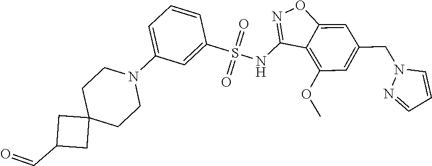 [SMILES: COc1cc(Cn2cccn2)cc2onc(NS(=O)(=O)c3cccc(N4CCC5(CC4)CC(C=O)C5)c3)c12]

To a solution of N-(6-((1H-pyrazol-1-yl)methyl)-4-methoxybenzo[d]isoxazol-3-yl)-3-(2-(hydroxymethyl)-7-azaspiro[3.5]nonan-7-yl)benzenesulfonamide (70.0 mg, 0.130 mmol) and triethylamine (0.127 mL, 0.911 mmol) in DMSO (0.50 mL) was added a solution of sulfur trioxide pyridine complex (124 mg, 0.781 mmol) in DMSO (0.50 mL). The reaction was stirred at rt for 1 h. The crude reaction solution was used directly in the next step without purification. LCMS calc. for C₂₇H₃₀N₅O₅S [M+H]⁺: m/z=536.2; Found 536.0.

### Step 5. N-(6-((1H-Pyrazol-1-yl)methyl)-4-methoxybenzo[d]isoxazol-3-yl)-3-(2-((4-(3-(2,4-dioxotetrahydropyrimidin-1(2H)-yl)-1-methyl-1H-indazol-6-yl)piperidin-1-yl)methyl)-7-azaspiro[3.5]nonan-7-yl)benzenesulfonamide

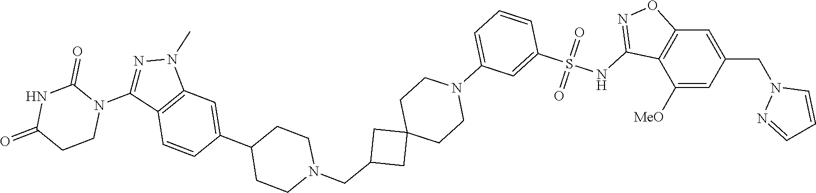 [SMILES: COc1cc(Cn2cccn2)cc2onc(NS(=O)(=O)c3cccc(N4CCC5(CC4)CC(CN4CCC(c6ccc7c(N8CCC(=O)NC8=O)nn(C)c7c6)CC4)C5)c3)c12]

To a solution of N-(6-((1H-Pyrazol-1-yl)methyl)-4-methoxybenzo[d]isoxazol-3-yl)-3-(2-formyl-7-azaspiro[3.5]nonan-7-yl)benzenesulfonamide (69.0 mg, 0.13 mmol) in DMSO (1.0 mL) (crude reaction from Step 4) was added a solution of 1-(1-methyl-6-(piperidin-4-yl)-1H-indazol-3-yl)dihydropyrimidine-2,4(1H,3H)-dione hydrochloride (which can be prepared as described in WO2022235945A1) (95.0 mg, 0.261 mmol) in DMSO (0.3 mL), followed by N,N-diisopropylethylamine (0.228 mL, 1.31 mmol). The reaction was stirred for 10 minutes then treated with sodium triacetoxyborohydride (111 mg, 0.523 mmol), followed by acetic acid (0.0747 mL, 1.31 mmol). The reaction was stirred at rt for 1 h then quenched with water, diluted with DMSO, filtered, and purified by prep-HPLC (Waters CSH-C18, 5 μm, 30×100 mm, 21.3-41.3% CH₃CN in H₂O with 0.2% TFA over 5 minutes) to afford the title compound (40.0 mg, 31.8% yield) as a white solid. ¹H NMR (400 MHz, DMSO) δ 10.77 (dt, J=9.1, 3.9 Hz, 1H), 10.56 (s, 1H), 9.35-9.12 (m, 1H), 7.87 (d, J=2.2 Hz, 1H), 7.62 (d, J=8.5 Hz, 1H), 7.49 (dd, J=6.4, 1.9 Hz, 2H), 7.43-7.34 (m, 2H), 7.34-7.28 (m, 1H), 7.22 (dd, J=8.4, 2.5 Hz, 1H), 7.02 (dd, J=8.6, 1.3 Hz, 1H), 6.79 (d, J=35.1 Hz, 2H), 6.30 (t, J=2.1 Hz, 1H), 5.44 (s, 2H), 3.98 (s, 3H), 3.91 (t, J=6.7 Hz, 4H), 3.86 (s, 3H), 3.32-3.15 (m, 4H), 3.09 (tt, J=10.0, 3.0 Hz, 3H), 3.02-2.90 (m, 2H), 2.75 (t, J=6.7 Hz, 3H), 2.20-2.02 (m, 4H), 1.97 (td, J=13.5, 3.3 Hz, 1H), 1.83-1.54 (m, 6H). LCMS calc. for C₄₄H₅₁N₁₀O₆S [M+H]⁺: m/z=847.4; Found 847.6.

### Example 36: N-(6-((1H-Pyrazol-1-yl)methyl)-4-methoxybenzo[d]isoxazol-3-yl)-3-(2-((4-(3-(2,6-dioxopiperidin-3-yl)-1-methyl-1H-indazol-6-yl)piperidin-1-yl)methyl)-7-azaspiro[3.5]nonan-7-yl)benzenesulfonamide

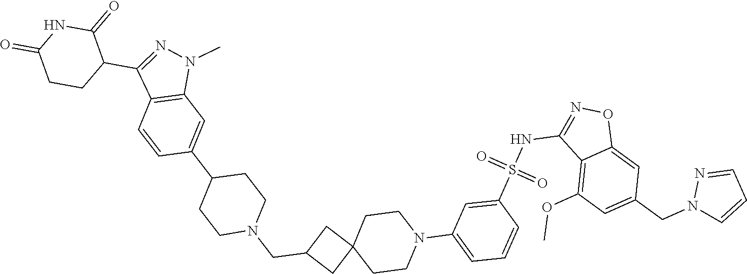 [SMILES: COc1cc(Cn2cccn2)cc2onc(NS(=O)(=O)c3cccc(N4CCC5(CC4)CC(CN4CCC(c6ccc7c(C8CCC(=O)NC8=O)nn(C)c7c6)CC4)C5)c3)c12]

The title compound was synthesized according to procedures analogous to Example 35, using 3-(1-methyl-6-(piperidin-4-yl)-1H-indazol-3-yl)piperidine-2,6-dione hydrochloride (which can be prepared as described in WO2024149214A1). LCMS calc. for C₄₅H₅₂N₉O₆S [M+H]⁺: m/z=846.4; Found 846.3

### Example 37: N-(6-((1H-Pyrazol-1-yl)methyl)-4-methoxybenzo[d]isoxazol-3-yl)-3-(2-((4-((1-(2,6-dioxopiperidin-3-yl)-3-methyl-2-oxo-2,3-dihydro-1H-benzo[d]imidazol-4-yl)ethynyl)piperidin-1-yl)methyl)-7-azaspiro[3.5]nonan-7-yl)benzenesulfonamide

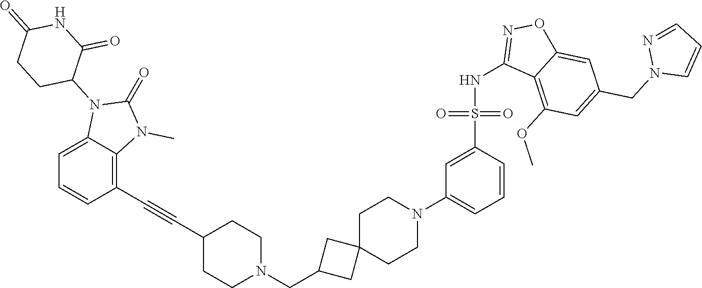 [SMILES: COc1cc(Cn2cccn2)cc2onc(NS(=O)(=O)c3cccc(N4CCC5(CC4)CC(CN4CCC(C#Cc6cccc7c6n(C)c(=O)n7C6CCC(=O)NC6=O)CC4)C5)c3)c12]

The title compound was synthesized according to procedures analogous to Example 35, using 3-(3-methyl-2-oxo-4-(piperidin-4-ylethynyl)-2,3-dihydro-1H-benzo[d]imidazol-1-yl)piperidine-2,6-dione hydrochloride (which can be prepared as described in WO 2023049790). LCMS calc. for C₄₇H₅₂N₉O₇S [M+H]⁺: m/z=886.4; Found 886.2.

### Example 38. N-(6-((1H-Pyrazol-1-yl)methyl)-4-methoxybenzo[d]isoxazol-3-yl)-4′-((4-(3-(2,4-dioxotetrahydropyrimidin-1(2H)-yl)-1-methyl-1H-indazol-6-yl)piperidin-1-yl)methyl)-2,4-dimethoxy-[1,1′-biphenyl]-3-sulfonamide

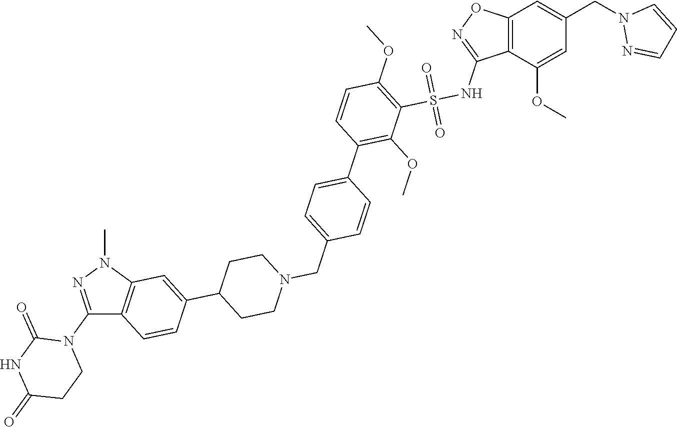 [SMILES: COc1ccc(-c2ccc(CN3CCC(c4ccc5c(N6CCC(=O)NC6=O)nn(C)c5c4)CC3)cc2)c(OC)c1S(=O)(=O)Nc1noc2cc(Cn3cccn3)cc(OC)c12]

### Step 1. N-(6-((1H-Pyrazol-1-yl)methyl)-4-methoxybenzo[d]isoxazol-3-yl)-4′-formyl-2,4-dimethoxy-[1,1′-biphenyl]-3-sulfonamide

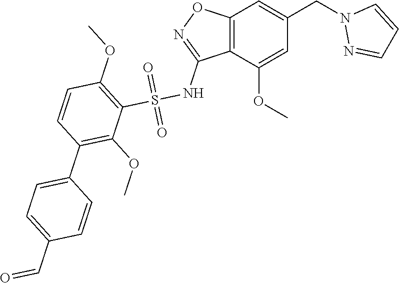 [SMILES: COc1ccc(-c2ccc(C=O)cc2)c(OC)c1S(=O)(=O)Nc1noc2cc(Cn3cccn3)cc(OC)c12]

To a nitrogen sparged solution of N-(6-((1H-pyrazol-1-yl)methyl)-4-methoxybenzo-[d]isoxazol-3-yl)-3-bromo-2,6-dimethoxybenzenesulfonamide (100 mg, 0.191 mmol) in 1,4-dioxane (1.7 mL) and water (0.30 mL) was added 4-(4,4,5,5-tetramethyl-1,3,2-dioxaborolan-2-yl)benzaldehyde (67 mg, 0.29 mmol), cesium carbonate (187 mg, 0.573 mmol), and [1,1′-bis(diphenylphosphino)ferrocene]dichloropalladium(II) (14 mg, 0.019 mmol). The reaction was stirred at 90° C. for 2 h. The reaction mixture was cooled to room temperature, poured into water, and extracted with EtOAc. The combined organic layers were washed with brine, dried over Na₂SO₄, filtered, and concentrated to afford the title compound (100 mg, 0.19 mmol, 98%) as a brown oil. LCMS calc. for C₂₇H₂₅N₄O₇S [M+H]⁺: m/z=549.1; Found 549.2.

### Step 2. N-(6-((1H-Pyrazol-1-yl)methyl)-4-methoxybenzo[d]isoxazol-3-yl)-4′-((4-(3-(2,4-dioxotetrahydropyrimidin-1(2H)-yl)-1-methyl-1H-indazol-6-yl)piperidin-1-yl)methyl)-2,4-dimethoxy-[1,1′-biphenyl]-3-sulfonamide

The title compound was synthesized according to procedures analogous to Example 35, Step 5. LCMS calc. for C₄₄H₄₆N₉O₈S [M+H]⁺: m/z=860.3; Found 860.6.

### Example 39. 3-(7-((1-(3-(2,6-Dioxopiperidin-3-yl)-1-methyl-1H-indazol-6-yl)piperidin-4-yl)methyl)-2,7-diazaspiro[3.5]nonan-2-yl)-5-fluoro-2,6-dimethoxy-N-(4-methoxy-6-(methoxymethyl)benzo[d]isoxazol-3-yl)benzenesulfonamide

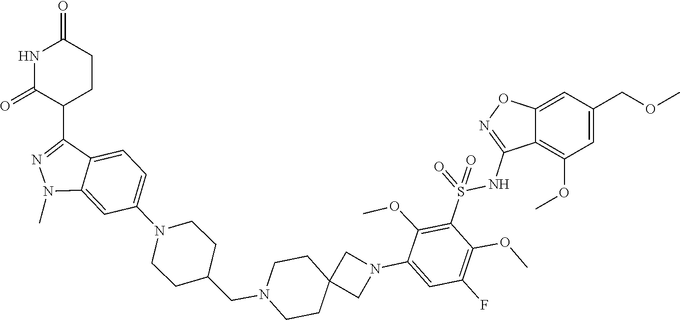 [SMILES: COCc1cc(OC)c2c(NS(=O)(=O)c3c(OC)c(F)cc(N4CC5(CCN(CC6CCN(c7ccc8c(C9CCC(=O)NC9=O)nn(C)c8c7)CC6)CC5)C4)c3OC)noc2c1]

### Step 1: 3-Chloro-5-fluoro-2,6-dimethoxy-N-(4-methoxy-6-(methoxymethyl)benzo-[d]isoxazol-3-yl)benzenesulfonamide

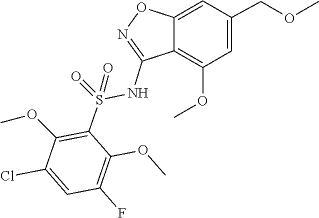

To a solution of 3-chloro-5-fluoro-2,6-dimethoxybenzenesulfonyl chloride (500 mg, 1.73 mmol) (described in Example 28, Steps 1-2) and 4-methoxy-6-(methoxymethyl)-benzo[d]isoxazol-3-amine (prepared as described by Stupple et al. al. WO2019243491 A1) (150 mg, 0.720 mmol) in THF (5 mL) was added potassium tert-butoxide (1.0 M in THF, 5.76 mL, 5.76 mmol) dropwise at 0° C. The mixture was stirred at 0° C. for 30 min, then quenched with brine (5 mL) and extracted with a 6:1 DCM/MeOH solution (2×20 mL). The combined organic phases were dried over MgSO₄, filtered, and concentrated. The crude material was purified by silica gel chromatography (20-80% ethyl acetate/hexanes) to afford the title compound (0.25 g, 76% yield) as a light orange solid. LCMS calc. for C₁₈H₁₉C₁FN₂O₇S [M+H]⁺: m/z=461.1; Found: 461.0.

### Step 2: 3-Fluoro-2,6-dimethoxy-N-(4-methoxy-6-(methoxymethyl)benzo[d]isoxazol-3-yl)-S-(2, 7-diazaspiro[3.5]nonan-2-yl)benzenesulfonamide

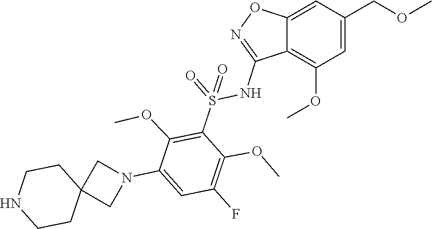 [SMILES: COCc1cc(OC)c2c(NS(=O)(=O)c3c(OC)c(F)cc(N4CC5(CCNCC5)C4)c3OC)noc2c1]

A mixture of tert-butyl 2,7-diazaspiro[3.5]nonane-7-carboxylate hydrochloride (39 mg, 0.15 mmol), sodium tert-butoxide (45 mg, 0.47 mmol), 3-chloro-5-fluoro-2,6-dimethoxy-N-(4-methoxy-6-(methoxymethyl)benzo[d]isoxazol-3-yl)benzenesulfonamide (43 mg, 0.09 mmol), and dicyclohexyl-[3,6-dimethoxy-2-[2,4,6-tri(propan-2-yl)phenyl]phenyl]phosphane; [2-[2-(methylamino)phenyl]phenyl]-methylsulfonyloxypalladium (BrettPhos Pd G4) (13 mg, 0.01 mmol), in 1,4-dioxane (3 mL) was sparged with nitrogen for 2 minutes. The reaction mixture was stirred at 90° C. for 1 hour. After cooling to room temperature, the mixture was diluted with 20% MeOH/DCM (4 mL), passed through a pad of silica gel eluting with 20% MeOH/DCM (50 mL), and concentrated. The resulting material was diluted with DCM (2 mL) and trifluoroacetic acid (360 μL, 4.67 mmol). The mixture was stirred at room temperature for 20 minutes. The volatiles were partially removed under reduced pressure, and the contents were diluted with MeCN (10 ml) and filtered through a 0.2 um PTFE syringe filter. The crude material was purified by prep-HPLC on a C18 column (24.5-44.5% CH₃CN/0.2% TFA (aq.)) to afford the title compound as a TFA salt (33 mg, 53% yield), a white solid. LCMS calc. for C₂₅H₃₂FN₄O₇S [M+H]⁺: m/z=551.2; Found: 551.2.

### Step 3: 3-(7-((1-(3-(2,6-Dioxopiperidin-3-yl)-1-methyl-1H-indazol-6-yl)piperidin-4-yl)methyl)-2, 7-diazaspiro[3.5]nonan-2-yl)-5-fluoro-2,6-dimethoxy-N-(4-methoxy-6-(methoxy-methyl)benzo[d]isoxazol-3-yl)benzenesulfonamide

To a solution of 1-[3-(2,6-dioxopiperidin-3-yl)-1-methylindazol-6-yl]piperidine-4-carbaldehyde (11 mg, 0.032 mmol) (prepared as described by Collier et. al. WO 2024/039901 A2) and 3-fluoro-2,6-dimethoxy-N-(4-methoxy-6-(methoxymethyl)benzo[d]isoxazol-3-yl)-5-(2,7-diazaspiro[3.5]nonan-2-yl)benzenesulfonamide (TFA salt) (7.0 mg, 0.011 mmol) in DMSO (0.50 mL) was added N,N-diisopropylethylamine (11 μL, 0.063 mmol) followed by acetic acid (1.8 μL, 0.032 mmol). The mixture was stirred for 10 min, then sodium triacetoxyborohydride (11 mg, 0.053 mmol) was added. Stirring continued at room temperature for 3 hours after which the mixture was diluted with DMSO (5 mL) and filtered through 0.2 um PTFE syringe filter. The crude material was purified by prep-HPLC on a C18 column (23.9-43.9% CH₃CN/0.2% TFA (aq.)) to afford the title compound as a TFA salt (7.5 mg, 71% yield), a white solid. LCMS calc. for C₄₄H₅₄FN₈O₉S [M+H]⁺: m/z=889.4; Found: 889.3.

### Example 40. 3-(7-((1-(3-(2,4-Dioxotetrahydropyrimidin-1(2H)-yl)-1-methyl-1H-indazol-6-yl)piperidin-4-yl)methyl)-2,7-diazaspiro[3.5]nonan-2-yl)-5-fluoro-2,6-dimethoxy-N-(4-methoxy-6-(methoxymethyl)benzo[d]isoxazol-3-yl)benzenesulfonamide

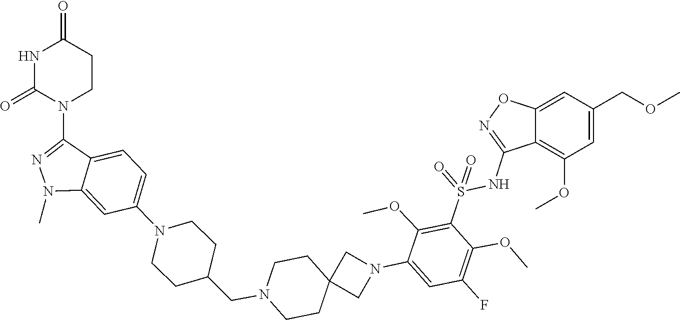 [SMILES: COCc1cc(OC)c2c(NS(=O)(=O)c3c(OC)c(F)cc(N4CC5(CCN(CC6CCN(c7ccc8c(N9CCC(=O)NC9=O)nn(C)c8c7)CC6)CC5)C4)c3OC)noc2c1]

The title compound was synthesized by procedures analogous to those described in Example 39, using 1-(3-(2,4-dioxotetrahydropyrimidin-1(2H)-yl)-1-methyl-1H-indazol-6-yl)piperidine-4-carbaldehyde (prepared as described by Fu et. al. US 2025/0049798 A1) instead of 1-[3-(2,6-dioxopiperidin-3-yl)-1-methylindazol-6-yl]piperidine-4-carbaldehyde in Step 3. LCMS calc. for C₄₃H₅₃FN₉O₉S [M+H]⁺: m/z=890.4; Found: 890.3.

### Example 41. 3-(7-((1-(3-(2,6-Dioxopiperidin-3-yl)-1-methyl-1H-pyrrolo[2,3-b]pyridin-6-yl)piperidin-4-yl)methyl)-2,7-diazaspiro[3.5]nonan-2-yl)-5-fluoro-2,6-dimethoxy-N-(4-methoxy-6-(methoxymethyl)benzo[d]isoxazol-3-yl)benzenesulfonamide

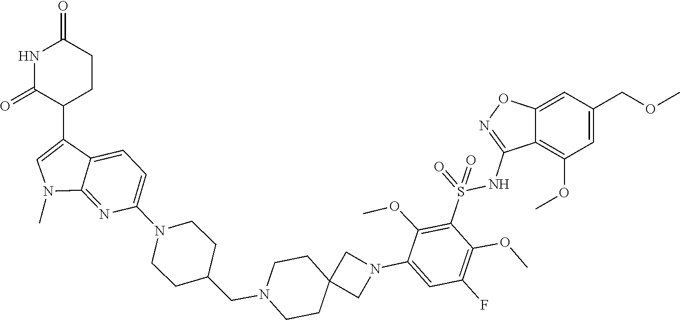 [SMILES: COCc1cc(OC)c2c(NS(=O)(=O)c3c(OC)c(F)cc(N4CC5(CCN(CC6CCN(c7ccc8c(C9CCC(=O)NC9=O)cn(C)c8n7)CC6)CC5)C4)c3OC)noc2c1]

The title compound was synthesized by procedures analogous to that described in Example 39, using 1-(3-(2,6-dioxopiperidin-3-yl)-1-methyl-1H-pyrrolo[2,3-b]pyridin-6-yl)piperidine-4-carbaldehyde (described in Example 9, Steps 1-4) instead of 1-[3-(2,6-dioxopiperidin-3-yl)-1-methylindazol-6-yl]piperidine-4-carbaldehyde in Step 3. LCMS calc. for C₄₄H₅₄FN₈O₉S [M+H]⁺: m/z=889.4; Found: 889.3.

### Example 42. 3-(2-((1-(3-(2,6-Dioxopiperidin-3-yl)-1-methyl-1H-indazol-6-yl)piperidin-4-yl)methyl)-1,2,3,4-tetrahydroisoquinolin-6-yl)-5-fluoro-2,6-dimethoxy-N-(4-methoxy-6-(methoxymethyl)benzo[d]isoxazol-3-yl)benzenesulfonamide

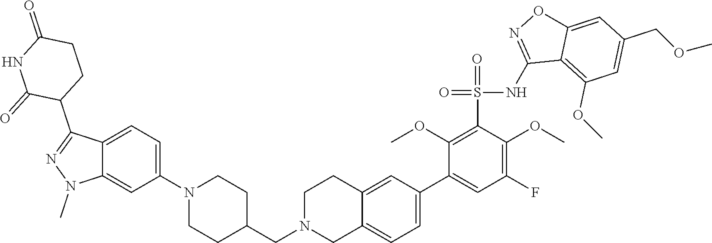 [SMILES: COCc1cc(OC)c2c(NS(=O)(=O)c3c(OC)c(F)cc(-c4ccc5c(c4)CCN(CC4CCN(c6ccc7c(C8CCC(=O)NC8=O)nn(C)c7c6)CC4)C5)c3OC)noc2c1]

### Step 1: 3-Fluoro-2,6-dimethoxy-N-(4-methoxy-6-(methoxymethyl)benzo[d]isoxazol-3-yl)-S-(1,2,3,4-tetrahydroisoquinolin-6-yl)benzenesulfonamide

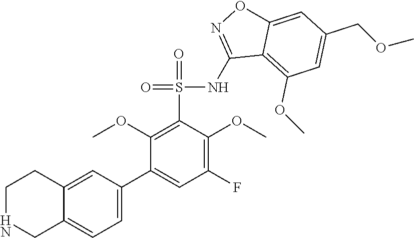 [SMILES: COCc1cc(OC)c2c(NS(=O)(=O)c3c(OC)c(F)cc(-c4ccc5c(c4)CCNC5)c3OC)noc2c1]

A solution of 3-chloro-5-fluoro-2,6-dimethoxy-N-(4-methoxy-6-(methoxymethyl)-benzo[d]isoxazol-3-yl)benzenesulfonamide (from Example 39, Step 1) (28.5 mg, 0.0618 mmol) in water (0.50 mL) and 1,4-dioxane (1.5 mL) was sparged with nitrogen for 3 minutes. Potassium carbonate (51.3 mg, 0.371 mmol), (2-(tert-butoxycarbonyl)-1,2,3,4-tetrahydroisoquinolin-6-yl)boronic acid (51.4 mg, 0.186 mmol), and XPhos Pd G4 (CAS 1599466-81-5, 8.0 mg, 0.093 mmol) were added. The mixture was sparged with nitrogen for an additional 3 minutes, then stirred at 90° C. for 40 minutes under nitrogen. After cooling to room temperature, the mixture was diluted with a 20% MeOH/DCM solution (4 mL) and passed through a pad of silica gel eluting with 20% MeOH/DCM (50 mL). The resulting material was concentrated, then diluted with DCM (2 mL). Trifluoroacetic acid (0.36 mL, 4.7 mmol) was added while stirring. The mixture was stirred at room temperature for 20 minutes. The volatiles were partially removed under reduced pressure. The contents were diluted with MeCN (10 ml) and filtered through a 0.2 um PTFE syringe filter. The crude material was purified by prep-HPLC on a C18 column (27.2-47.2% CH₃CN/0.2% TFA (aq.)) to afford the title compound as a TFA salt (23 mg, 55% yield), a white solid. LCMS calc. for C₂₇H₂₉FN₃O₇S [M+H]⁺: m/z=558.2; Found: 558.3.

### Step 2: 3-(2-((1-(3-(2,6-Dioxopiperidin-3-yl)-1-methyl-1H-indazol-6-yl)piperidin-4-yl)-methyl)-1,2,3,4-tetrahydroisoquinolin-6-yl)-5-fluoro-2,6-dimethoxy-N-(4-methoxy-6-(methoxymethyl)benzo[d]isoxazol-3-yl)benzenesulfonamide

The title compound was synthesized by procedures analogous to that described in Example 39, Step 3. LCMS calc. for C₄₆H₅₁FN₇O₉S [M+H]⁺: m/z=896.4; Found: 896.3.

### Example 43. 3-(2-((1-(3-(2,4-Dioxotetrahydropyrimidin-1(2H)-yl)-1-methyl-1H-indazol-6-yl)piperidin-4-yl)methyl)-1,2,3,4-tetrahydroisoquinolin-6-yl)-5-fluoro-2,6-dimethoxy-N-(4-methoxy-6-(methoxymethyl)benzo[d]isoxazol-3-yl)benzenesulfonamide

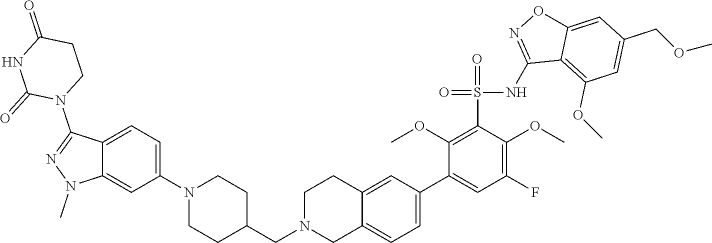 [SMILES: COCc1cc(OC)c2c(NS(=O)(=O)c3c(OC)c(F)cc(-c4ccc5c(c4)CCN(CC4CCN(c6ccc7c(N8CCC(=O)NC8=O)nn(C)c7c6)CC4)C5)c3OC)noc2c1]

The title compound was synthesized by procedures analogous to that described in Example 39, Step 3. LCMS calc. for C₄₅H₅₀FN₈O₉S [M+H]⁺: m/z=897.3; Found: 897.4.

### Example 44. 4′-((4-(3-(2,6-Dioxopiperidin-3-yl)-1-methyl-1H-indazol-6-yl)piperidin-1-yl)methyl)-5-fluoro-2,4-dimethoxy-N-(4-methoxy-6-(methoxymethyl)benzo[d]isoxazol-3-yl)-3′-methyl-[1,1′-biphenyl]-3-sulfonamide

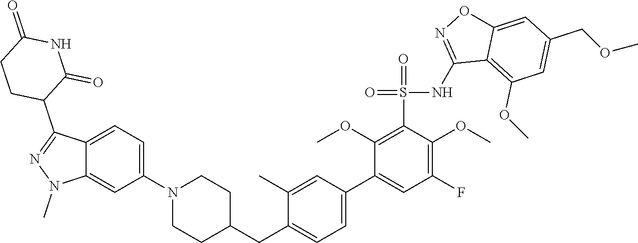 [SMILES: COCc1cc(OC)c2c(NS(=O)(=O)c3c(OC)c(F)cc(-c4ccc(CC5CCN(c6ccc7c(C8CCC(=O)NC8=O)nn(C)c7c6)CC5)c(C)c4)c3OC)noc2c1]

### Step 1: 5-Fluoro-4′-formyl-2,4-dimethoxy-N-(4-methoxy-6-(methoxymethyl)-benzo[d]isoxazol-3-yl)-3′-methyl-[1,1′-biphenyl]-3-sulfonamide

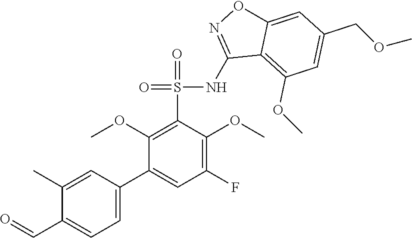 [SMILES: COCc1cc(OC)c2c(NS(=O)(=O)c3c(OC)c(F)cc(-c4ccc(C=O)c(C)c4)c3OC)noc2c1]

A solution of 3-chloro-5-fluoro-2,6-dimethoxy-N-(4-methoxy-6-(methoxymethyl)-benzo[d]isoxazol-3-yl)benzenesulfonamide (34.0 mg, 0.07 mmol) (from Example 39, Step 1) in water (0.20 mL) and 1,4-dioxane (1.5 mL) was sparged with nitrogen for 3 minutes. Potassium carbonate (61 mg, 0.44 mmol), XPhos Pd G4 (CAS 1599466-81-5, 10 mg, 0.01 mmol), and (4-formyl-3-methylphenyl)boronic acid (60.5 mg, 0.369 mmol) were added. The mixture was purged with nitrogen for 3 minutes, and then stirred at 90° C. for 40 minutes. After cooling to room temperature, the mixture was diluted with a 20% MeOH/DCM solution (4 mL), passed through a pad of silica gel eluting with 20% MeOH/DCM (50 mL), and concentrated The crude material was purified by prep-HPLC (51.9-71.9% CH₃CN/0.2% TFA (aq.)) to afford the title compound as a TFA salt (12 mg, 25% yield), a white solid. LCMS calc. for C₂₆H₂₆FN₂O₈S [M+H]⁺: m/z=545.1; Found: 545.0.

### Step 2: 4′-((4-(3-(2,6-Dioxopiperidin-3-yl)-1-methyl-1H-indazol-6-yl)piperidin-1-yl)-methyl)-5-fluoro-2,4-dimethoxy-N-(4-methoxy-6-(methoxymethyl)benzo[d]isoxazol-3-yl)-3′-methyl-[1,1′-biphenyl]-3-sulfonamide

The title compound was synthesized by procedures analogous to that described in Example 39, using 3-(1-methyl-6-(piperidin-4-yl)-1H-indazol-3-yl)piperidine-2,6-dione hydrochloride (prepared as described by Nasveschuk et. al. WO 2022/261250 A1) instead of 1-[3-(2,6-dioxopiperidin-3-yl)-1-methylindazol-6-yl]piperidine-4-carbaldehyde in Step 3. LCMS calc. for C₄₄H₄₈FN₆O₉S [M+H]⁺: m/z=855.3; Found: 855.2.

### Example 45. 4′-((4-(3-(2,4-Dioxotetrahydropyrimidin-1(2H)-yl)-1-methyl-1H-indazol-6-yl)piperidin-1-yl)methyl)-5-fluoro-2,4-dimethoxy-N-(4-methoxy-6-(methoxymethyl)-benzo[d]isoxazol-3-yl)-3′-methyl-[1,1′-biphenyl]-3-sulfonamide

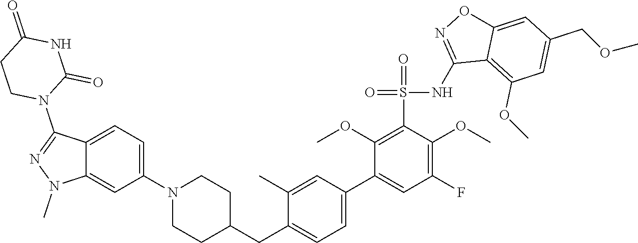 [SMILES: COCc1cc(OC)c2c(NS(=O)(=O)c3c(OC)c(F)cc(-c4ccc(CC5CCN(c6ccc7c(N8CCC(=O)NC8=O)nn(C)c7c6)CC5)c(C)c4)c3OC)noc2c1]

The title compound was synthesized by procedures analogous to that described in Example 39, using 1-(1-methyl-6-(piperidin-4-yl)-1H-indazol-3-yl)dihydropyrimidine-2,4(1H,3H)-dione hydrochloride (prepared as described by Nasveschuk et. al. WO 2022/261250) instead of 1-[3-(2,6-dioxopiperidin-3-yl)-1-methylindazol-6-yl]piperidine-4-carbaldehyde in Step 3. LCMS calc. for C₄₃H₄₇FN₇O₉S [M+H]⁺: m/z=856.3; Found: 856.3.

### Example 46. N-(6-((1H-Pyrazol-1-yl)methyl)-4-methoxybenzo[d]isoxazol-3-yl)-3-(7-((1-(3-(2,4-dioxotetrahydropyrimidin-1(2H)-yl)-1-methyl-1H-indazol-6-yl)piperidin-4-yl)methyl)-2,7-diazaspiro[3.5]nonan-2-yl)-5-fluoro-2,6-dimethoxybenzenesulfonamide

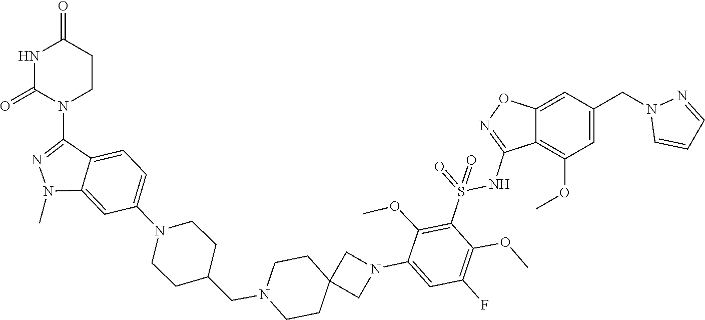 [SMILES: COc1c(F)cc(N2CC3(CCN(CC4CCN(c5ccc6c(N7CCC(=O)NC7=O)nn(C)c6c5)CC4)CC3)C2)c(OC)c1S(=O)(=O)Nc1noc2cc(Cn3cccn3)cc(OC)c12]

### Step 1: N-(6-((1H-Pyrazol-1-yl)methyl)-4-methoxybenzo[d]isoxazol-3-yl)-3-(7-((1-(3-(2,4-dioxotetrahydropyrimidin-1(2H)-yl)-1-methyl-1H-indazol-6-yl)piperidin-4-yl)methyl)-2, 7-diazaspiro[3.5]nonan-2-yl)-5-fluoro-2,6-dimethoxybenzenesulfonamide

To a solution of 1-(3-(2,4-dioxotetrahydropyrimidin-1(2H)-yl)-1-methyl-1H-indazol-6-yl)piperidine-4-carbaldehyde (prepared as described by Fu et. al. US 2025/0049798 A1) (8.0 mg, 22 μmol) and N-(6-((1H-pyrazol-1-yl)methyl)-4-methoxybenzo[d]isoxazol-3-yl)-3-fluoro-2,6-dimethoxy-5-(2,7-diazaspiro[3.5]nonan-2-yl)benzenesulfonamide (TFA salt) (6.3 mg, 8.9 μmol) (described in Example 28, Steps 1-4) in DMSO (0.50 mL) was added N,N-diisopropylethylamine (15.7 μL, 89 μmol) followed by acetic acid (2.6 μL, 45 μmol). The mixture was stirred for 10 min, then sodium triacetoxyborohydride (11.4 mg, 53.9 μmol) was added. The mixture continued to stir at room temperature for 3 h after which the mixture was diluted with DMSO (5 mL) and filtered through 0.2 um PTFE syringe filter. The crude material was purified by prep-HPLC on a C18 column (21.6-41.6% CH₃CN/0.2% TFA (aq.)) to afford the title compound as TFA salt (7.6 mg, 81% yield), a white solid. LCMS calc. for C₄₅H₅₃FN₁₁O₈S [M+H]⁺: m/z=926.4; Found: 926.2.

### Example 47. rac-3-((cis)-3-((1-(3-(2,4-Dioxotetrahydropyrimidin-1(2H)-yl)-1-methyl-1H-indazol-6-yl)piperidin-4-yl)methyl)-3,7-diazabicyclo[4.2.0]octan-7-yl)-5-fluoro-2,6-dimethoxy-N-(4-methoxy-6-(methoxymethyl)benzo[d]isoxazol-3-yl)benzenesulfonamide

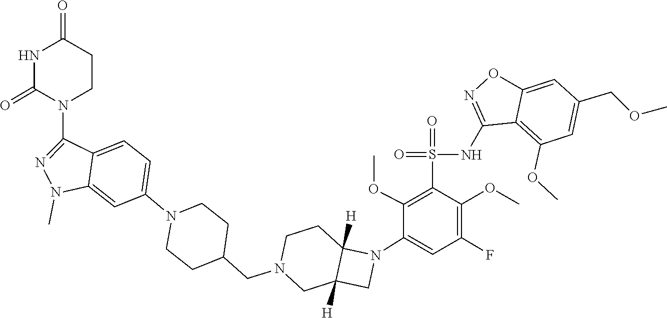 [SMILES: COCc1cc(OC)c2c(NS(=O)(=O)c3c(OC)c(F)cc(N4C[C@@H]5CN(CC6CCN(c7ccc8c(N9CCC(=O)NC9=O)nn(C)c8c7)CC6)CC[C@@H]54)c3OC)noc2c1]

### Step 1: rac-3-((cis)-3, 7-Diazabicyclo[4.2.0]octan-7-yl)-5-fluoro-2,6-dimethoxy-N-(4-methoxy-6-(methoxymethyl)benzo[d]isoxazol-3-yl)benzenesulfonamide

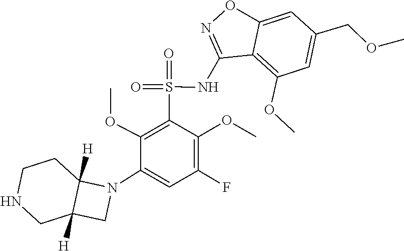 [SMILES: COCc1cc(OC)c2c(NS(=O)(=O)c3c(OC)c(F)cc(N4C[C@@H]5CNCC[C@@H]54)c3OC)noc2c1]

The title compound was synthesized by procedures analogous to that described in Example 39, Step 2, using rac-tert-butyl (cis)-3,7-diazabicyclo[4.2.0]octane-3-carboxylate hydrochloride instead of tert-butyl 2,7-diazaspiro[3.5]nonane-7-carboxylate hydrochloride. LCMS calc. for C₂₄H₃₀FN₄O₇S [M+H]⁺: m/z=537.2; Found: 537.1.

### Step 2: rac-3-((cis)-3-((1-(3-(2,4-Dioxotetrahydropyrimidin-1(2H)-yl)-1-methyl-1H-indazol-6-yl)piperidin-4-yl)methyl)-3, 7-diazabicyclo[4.2.0]octan-7-yl)-5-fluoro-2,6-dimethoxy-N-(4-methoxy-6-(methoxymethyl)benzo[d]isoxazol-3-yl)benzenesulfonamide

The racemic title compound was synthesized by procedures analogous to that described in Example 39, Step 3. LCMS calc. for C₄₂H₅₁FN₉O₉S [M+H]⁺: m/z=876.3; Found: 876.3.

### Example 48. 3-((1-(3-(2,6-Dioxopiperidin-3-yl)-1-methyl-1H-pyrrolo[2,3-b]pyridin-6-yl)piperidin-4-yl)methyl)-3,7-diazabicyclo[4.2.0]octan-7-yl)-5-fluoro-2,6-dimethoxy-N-(4-methoxy-6-(methoxymethyl)benzo[d]isoxazol-3-yl)benzenesulfonamide (mixture of four stereoisomers)

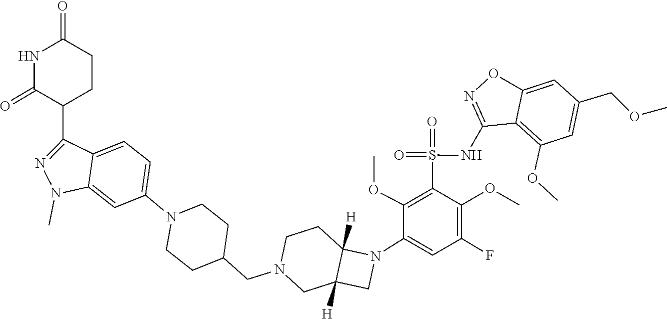 [SMILES: COCc1cc(OC)c2c(NS(=O)(=O)c3c(OC)c(F)cc(N4C[C@@H]5CN(CC6CCN(c7ccc8c(C9CCC(=O)NC9=O)nn(C)c8c7)CC6)CC[C@@H]54)c3OC)noc2c1]

The mixture of 4 diastereomeric title compounds were synthesized by procedures analogous to that described in Example 39, Step 3. LCMS calc. for C₄₃H₅₂FN₈O₉S [M+H]⁺: m/z=875.4; Found: 875.3.

### Example 49. rac-3-((cis)-3-((1-(3-(2,4-Dioxotetrahydropyrimidin-1(2H)-yl)-1-methyl-1H-indazol-6-yl)piperidin-4-yl)methyl)-3,7-diazabicyclo[4.2.0]octan-7-yl)-2-methoxy-N-(4-methoxy-6-(methoxymethyl)benzo[d]isoxazol-3-yl)-5-(trifluoromethyl)benzene-sulfonamide

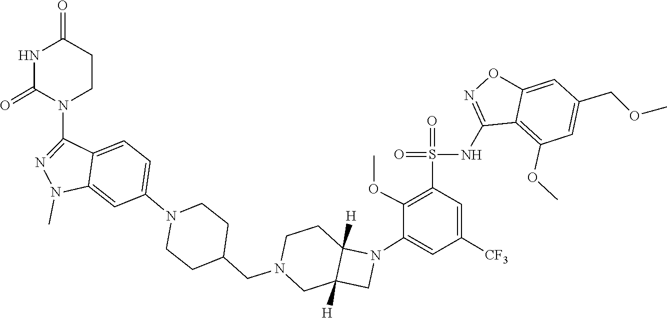 [SMILES: COCc1cc(OC)c2c(NS(=O)(=O)c3cc(C(F)(F)F)cc(N4C[C@@H]5CN(CC6CCN(c7ccc8c(N9CCC(=O)NC9=O)nn(C)c8c7)CC6)CC[C@@H]54)c3OC)noc2c1]

### Step 1: Benzyl(3-bromo-2-methoxy-5-(trifluoromethyl)phenyl)sulfane

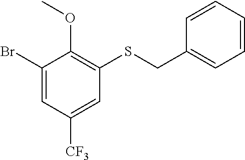 [SMILES: COc1c(Br)cc(C(F)(F)F)cc1SCc1ccccc1]

Tris(dibenzylideneacetone)dipalladium(0) (63.4 mg, 0.0692 mmol), 9,9-dimethyl-4,5-bis(diphenylphosphino)xanthene (80.0 mg, 0.138 mmol), 1,3-dibromo-2-methoxy-5-(trifluoromethyl)benzene (924 mg, 2.77 mmol), phenylmethanethiol (340 μL, 2.91 mmol), N,N-diisopropylethylamine (964 μL, 5.53 mmol), and 1,4-dioxane (10 mL) were added to a 40 mL vial. The vial was sealed with a septum cap and the mixture was sparged with nitrogen via a needle for 5 minutes. The mixture was then stirred at 100° C. for 3 hours. After cooling to room temperature, it was passed through a pad of silica gel (10 g) eluting with ethyl acetate (80 mL). The volatiles were removed under reduced pressure. The crude material was purified by flash silica gel column chromatography (0-20% DCM/hexanes) to afford the title compound (0.76 g, 72% yield) as colorless oil. ¹H NMR (400 MHz, CDCl₃) δ 7.63 (dd, J=2.1, 0.9 Hz, 1H), 7.43-7.24 (m, 6H), 4.17 (s, 2H), 3.93 (s, 3H). ¹⁹F NMR (376 MHz, CDCl₃) δ −62.36.

### Step 2: 3-Bromo-2-methoxy-5-(trifluoromethyl)benzenesulfonyl chloride

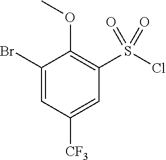 [SMILES: COc1c(Br)cc(C(F)(F)F)cc1S(=O)(=O)Cl]

The title compound was synthesized by procedures analogous to that described in Example 28, Step 2. ¹H NMR (400 MHz, CDCl₃) δ 8.23 (m, 1H), 8.20 (m, 1H), 4.22 (s, 3H). 19F NMR (376 MHz, CDCl₃) δ −62.40.

### Step 3: 3-Bromo-2-methoxy-N-(4-methoxy-6-(methoxymethyl)benzo[d]isoxazol-3-yl)-5-(trifluoromethyl)benzenesulfonamide

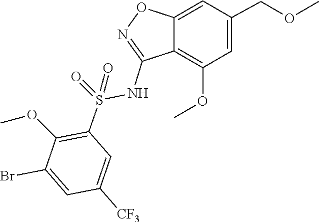

3-Bromo-2-methoxy-5-(trifluoromethyl)benzenesulfonyl chloride (0.31 g, 0.86 mmol), 4-methoxy-6-(methoxymethyl)benzo[d]isoxazol-3-amine (72 mg, 0.35 mmol), and THF (1.7 mL) were added to a 20 mL vial. The vial was placed in an ice bath and the mixture was stirred at 0° C. for 5 minutes. Next, potassium t-butoxide (1.0 M in THF, 2.42 mL, 2.42 mmol) was added drop wise. The mixture was stirred at 0° C. for 30 minutes and then quenched with brine (5 mL) and extracted with a 6:1 DCM/MeOH solution (2×20 mL). The combined organic phase was dried over anhydrous magnesium sulfate, filtered, and concentrated under reduced pressure. The crude material was purified by flash silica gel column chromatography (0-5% MeOH/DCM) to afford the title compound (88 mg, 48% yield) as off white solid. LCMS calc. for C₁₈H₁₇BrF₃N₂O₆S [M+H]⁺: m/z=525.0 and 526.0; Found: 524.9 and 525.9.

### Step 4: rac-3-((cis)-3-((1-(3-(2,4-Dioxotetrahydropyrimidin-1(2H)-yl)-1-methyl-1H-indazol-6-yl)piperidin-4-yl)methyl)-3, 7-diazabicyclo[4.2.0]octan-7-yl)-2-methoxy-N-(4-methoxy-6-(methoxymethyl)benzo[d]isoxazol-3-yl)-5-(trifluoromethyl)benzenesulfonamide

The title compound was synthesized by procedures analogous to that described in Example 39, Step 2 and 3. LCMS calc. for C₄₂H₄₉F₃N₉O₈S [M+H]⁺: m/z=896.3; Found: 896.2.

### Example 50. 3-((1-(3-(2,6-Dioxopiperidin-3-yl)-1-methyl-1H-pyrrolo[2,3-b]pyridin-6-yl)piperidin-4-yl)methyl)-3,7-diazabicyclo[4.2.0]octan-7-yl)-2-methoxy-N-(4-methoxy-6-(methoxymethyl)benzo[d]isoxazol-3-yl)-5-(trifluoromethyl)benzenesulfonamide (mixture of four stereoisomers)

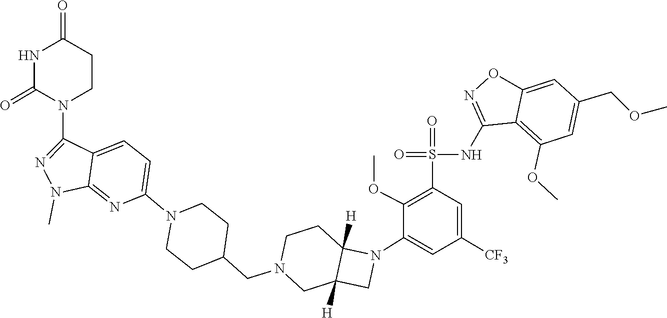 [SMILES: COCc1cc(OC)c2c(NS(=O)(=O)c3cc(C(F)(F)F)cc(N4C[C@@H]5CN(CC6CCN(c7ccc8c(N9CCC(=O)NC9=O)nn(C)c8n7)CC6)CC[C@@H]54)c3OC)noc2c1]

The mixture of 4 diastereomeric title compounds were synthesized by procedures analogous to that described in Example 39, Step 3. LCMS calc. for C₄₃H₅₀F₃N₈O₈S [M+H]⁺: m/z=895.3; Found: 895.3.

### Example 51. N-(6-((1H-pyrazol-1-yl)methyl)-4-methoxybenzo[d]isoxazol-3-yl)-3-(6-((1-(3-(2,4-dioxotetrahydropyrimidin-1(2H)-yl)-1-methyl-1H-indazol-6-yl)piperidin-4-yl)methyl)-5,6,7,8-tetrahydro-1,6-naphthyridin-2-yl)-5-fluoro-2,6-dimethoxybenzene-sulfonamide

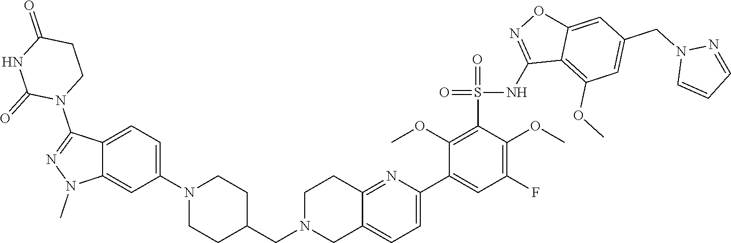 [SMILES: COc1c(F)cc(-c2ccc3c(n2)CCN(CC2CCN(c4ccc5c(N6CCC(=O)NC6=O)nn(C)c5c4)CC2)C3)c(OC)c1S(=O)(=O)Nc1noc2cc(Cn3cccn3)cc(OC)c12]

### Step 1: N-(6-((1H-pyrazol-1-yl)methyl)-4-methoxybenzo[d]isoxazol-3-yl)-3-bromo-5-fluoro-2,6-dimethoxy-N-((2-(trimethylsilyl)ethoxy)methyl)benzenesulfonamide

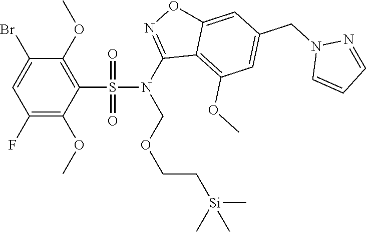 [SMILES: COc1c(F)cc(Br)c(OC)c1S(=O)(=O)N(COCC[Si](C)(C)C)c1noc2cc(Cn3cccn3)cc(OC)c12]

N-(6-((1H-pyrazol-1-yl)methyl)-4-methoxybenzo[d]isoxazol-3-yl)-3-bromo-5-fluoro-2,6-dimethoxybenzenesulfonamide (300 mg, 0.55 mmol), THF (10 mL), and sodium hydride (88.7 mg, 2.22 mmol) were stirred at rt for 5 min. (2-(chloromethoxy)ethyl)trimethyl-silane (393 μL, 2.22 mmol) was added next. The mixture was stirred at rt for 2 hours and at 40° C. overnight. The reaction was quenched by brine (30 mL) and extracted with DCM (3×40 mL). The combined organic phase was dried over anhydrous MgSO₄, filtered, and concentrated under reduced pressure. The crude material was purified by flash silica gel column chromatography (0 to 10% MeOH/DCM) to afford the title compound (353 mg, 95% yield) as viscous gum which solidified slowly to a yellow solid. LCMS calc. for C₂₆H₃₃BrFN₄O₇SSi [M+H]⁺: m/z=671.1, 673.1; Found: 671.1, 673.0.

### Step 2: N-(6-((1H-pyrazol-1-yl)methyl)-4-methoxybenzo[d]isoxazol-3-yl)-3-fluoro-2,6-dimethoxy-5-(4,4,5,5-tetramethyl-1,3,2-dioxaborolan-2-yl)-N-((2-(trimethylsilyl)ethoxy)-methyl)benzenesulfonamide

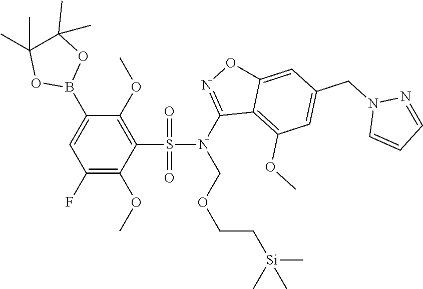 [SMILES: COc1c(F)cc(B2OC(C)(C)C(C)(C)O2)c(OC)c1S(=O)(=O)N(COCC[Si](C)(C)C)c1noc2cc(Cn3cccn3)cc(OC)c12]

N-(6-((1H-pyrazol-1-yl)methyl)-4-methoxybenzo[d]isoxazol-3-yl)-3-bromo-5-fluoro-2,6-dimethoxy-N-((2-(trimethylsilyl)ethoxy)methyl)benzenesulfonamide (25 mg, 0.04 mmol), 2-Isopropoxy-4,4,5,5-tetramethyl-1,3,2-dioxaborolane (30 μL, 0.15 mmol), and THF (1 mL) were cooled to 0° C. in an ice/water bath and stirred under nitrogen. Isopropyl-magnesium chloride lithium chloride complex solution (143 μL, 0.19 mmol, 1.3 M in THF) was added drop wise. The mixture was stirred for 1 hour at 0° C. and then quenched with saturated NH₄Cl_{(aq)} and brine. The mixture was extracted with ethyl acetate (3×10 mL), dried over anhydrous MgSO₄, filtered, and concentrated under reduced pressure. The crude material was purified by flash silica gel column chromatography (0 to 100% ethyl acetate/hexanes) to afford the title compound (23 mg, 86% yield) as colorless oil. LCMS calc. for C₃₂H₄₅BrFN₄O₉SSi [M+H]⁺: m/z=719.3; Found: 719.2.

### Step 3: N-(6-((1H-pyrazol-1-yl)methyl)-4-methoxybenzo[d]isoxazol-3-yl)-3-fluoro-2,6-dimethoxy-5-(5, 6,7,8-tetrahydro-1,6-naphthyridin-2-yl)benzenesulfonamide

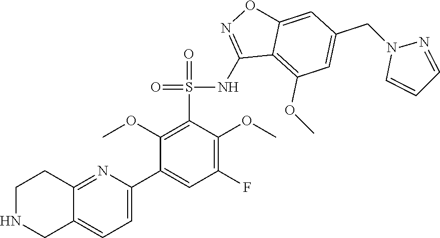 [SMILES: COc1c(F)cc(-c2ccc3c(n2)CCNC3)c(OC)c1S(=O)(=O)Nc1noc2cc(Cn3cccn3)cc(OC)c12]

N-(6-((1H-pyrazol-1-yl)methyl)-4-methoxybenzo[d]isoxazol-3-yl)-3-fluoro-2,6-dimethoxy-5-(4,4,5,5-tetramethyl-1,3,2-dioxaborolan-2-yl)-N-((2-(trimethylsilyl)ethoxy)-methyl)benzenesulfonamide (53.0 mg, 0.07 mmol), water (0.20 mL), and 1,4-dioxane (2 mL) were sparged with nitrogen for 3 minutes. Potassium Carbonate (40.8 mg, 0.29 mmol), SPhos Pd G2 (10.6 mg, 0.01 mmol), and tert-butyl 2-chloro-7,8-dihydro-1,6-naphthyridine-6(5H)-carboxylate (39.6 mg, 0.15 mmol) were added next. The mixture was stirred at 80° C. under nitrogen for 1 h, cooled to rt, diluted with a 6:1 solution of DCM/MeOH (5 mL), and passed through a pad of silica gel eluting with a 6:1 solution of DCM/MeOH (50 mL). The volatiles were removed under reduced pressure. The resulting material was dissolved in DCM (1 mL) and 2,2,2-trifluoroacetic acid (560 μL, 7.37 mmol), and stirred at rt for 3 hours. The crude material was diluted with MeCN (3 mL) and purified by prep-HPLC on a C18 column (24.6-44.6% CH₃CN/0.2% TFA (aq.)) to afford the title compound (32 mg, 61% yield) as TFA salt as white solid. LCMS calc. for C₂₈H₂₈FN₆O₆S [M+H]⁺: m/z=595.2; Found: 595.2.

### Step 4: N-(6-((1H-pyrazol-1-yl)methyl)-4-methoxybenzo[d]isoxazol-3-yl)-3-(6-((1-(3-(2,4-dioxotetrahydropyrimidin-1(2H)-yl)-1-methyl-1H-indazol-6-yl)piperidin-4-yl)methyl)-5,6,7,8-tetrahydro-1,6-naphthyridin-2-yl)-5-fluoro-2,6-dimethoxybenzenesulfonamide

The title compound was synthesized by procedures analogous to that described in Example 39, Step 3. LCMS calc. for C₄₆H₄₉FN₁₁O₈S [M+H]⁺: m/z=934.4; Found: 934.2.

### Example 52. N-(6-((1H-pyrazol-1-yl)methyl)-4-methoxybenzo[d]isoxazol-3-yl)-3-(7-((1-(4-(2,6-dioxopiperidin-3-yl)phenyl)piperidin-4-yl)methyl)-2,7-diazaspiro[3.5]nonan-2-yl)-5-fluoro-2,6-dimethoxybenzenesulfonamide

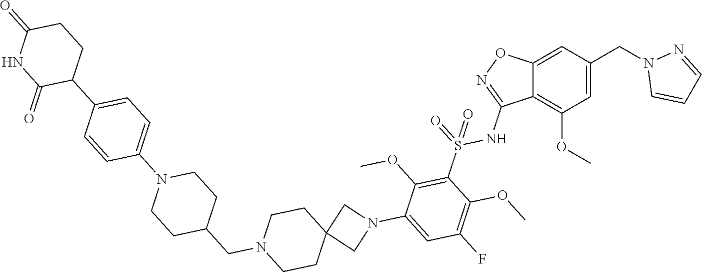 [SMILES: COc1c(F)cc(N2CC3(CCN(CC4CCN(c5ccc(C6CCC(=O)NC6=O)cc5)CC4)CC3)C2)c(OC)c1S(=O)(=O)Nc1noc2cc(Cn3cccn3)cc(OC)c12]

The title compound was synthesized by procedures analogous to that described in Example 39, using 1-(4-(2,6-dioxopiperidin-3-yl)phenyl)piperidine-4-carbaldehyde (CAS #: 2736511-32-1) instead of 1-[3-(2,6-dioxopiperidin-3-yl)-1-methylindazol-6-yl]piperidine-4-carbaldehyde in Step 3. LCMS calc. for C₄₄H₅₁FN₈O₈S [M+H]⁺: m/z=871.4; Found: 871.5.

### Example 53: rac-3-(cis)-N-(6-((1H-pyrazol-1-yl)methyl)-4-methoxybenzo[d]isoxazol-3-yl)-3-((1-(3-(2,4-dioxotetrahydropyrimidin-1(2H)-yl)-1-methyl-1H-indazol-6-yl)-piperidin-4-yl)methyl)-3,7-diazabicyclo[4.2.0]octan-7-yl)-5-fluoro-2,6-dimethoxy-benzenesulfonamide

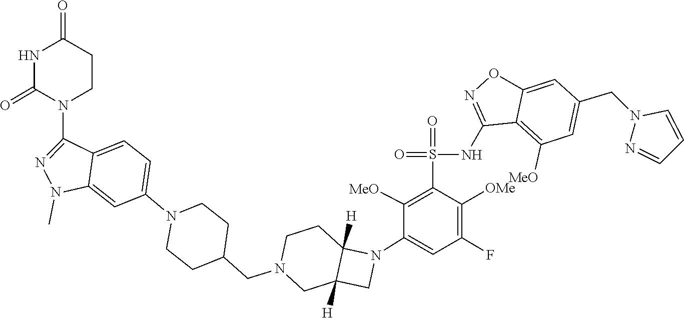 [SMILES: COc1c(F)cc(N2C[C@@H]3CN(CC4CCN(c5ccc6c(N7CCC(=O)NC7=O)nn(C)c6c5)CC4)CC[C@@H]32)c(OC)c1S(=O)(=O)Nc1noc2cc(Cn3cccn3)cc(OC)c12]

The title compound was synthesized by procedures analogous to that described in Example 47, using rac-3-(cis)-N-(6-((1H-pyrazol-1-yl)methyl)-4-methoxybenzo[d]isoxazol-3-yl)-3,7-diazabicyclo[4.2.0]octan-7-yl)-5-fluoro-2,6-dimethoxybenzenesulfonamide instead of rac-3-((cis)-3,7-Diazabicyclo[4.2.0]octan-7-yl)-5-fluoro-2,6-dimethoxy-N-(4-methoxy-6-(methoxymethyl)benzo[d]isoxazol-3-yl)benzenesulfonamide. LCMS calc. for C₄₄H₅₁FN₁₁O₈S [M+H]⁺: m/z=912.4; Found: 912.0.

### Example 54. N-(6-((1H-Pyrazol-1-yl)methyl)-4-methoxybenzo[d]isoxazol-3-yl)-3-(7-((4-(3-(2,4-dioxotetrahydropyrimidin-1(2H)-yl)-1-methyl-1H-indazol-6-yl)piperazin-1-yl)methyl)-2-azaspiro[3.5]nonan-2-yl)benzenesulfonamide

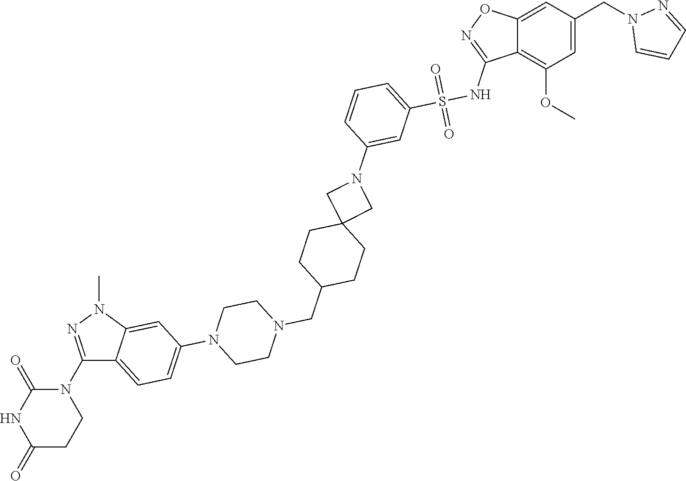 [SMILES: COc1cc(Cn2cccn2)cc2onc(NS(=O)(=O)c3cccc(N4CC5(CCC(CN6CCN(c7ccc8c(N9CCC(=O)NC9=O)nn(C)c8c7)CC6)CC5)C4)c3)c12]

The title compound was synthesized according to procedures analogous to Example 35, Steps 1-5 using tert-butyl 7-(((di-tert-butyl(phenyl)silyl)oxy)methyl)-2-azaspiro[3.5]-nonane-2-carboxylate and 1-(1-methyl-6-(piperazin-1-yl)-1H-indazol-3-yl)dihydropyrimidine-2,4(1H,3H)-dione (which can be prepared as described in WO2023283372 A1). LCMS calc. for C₄₃H₅₀N₁₁O₆S [M+H]⁺: m/z=848.4; Found: 848.3.

### Example 55. N-(6-((1H-Pyrazol-1-yl)methyl)-4-methoxybenzo[d]isoxazol-3-yl)-3-(7-((4-(4-(2,6-dioxopiperidin-3-yl)phenyl)piperidin-1-yl)methyl)-2-azaspiro[3.5]nonan-2-yl)-5-fluoro-2,6-dimethoxybenzenesulfonamide

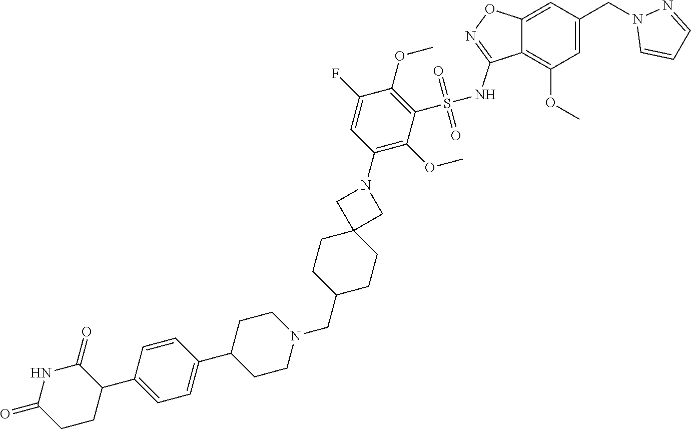 [SMILES: COc1c(F)cc(N2CC3(CCC(CN4CCC(c5ccc(C6CCC(=O)NC6=O)cc5)CC4)CC3)C2)c(OC)c1S(=O)(=O)Nc1noc2cc(Cn3cccn3)cc(OC)c12]

### Step 1. 3-Fluoro-2,6-dimethoxybenzenesulfonyl chloride

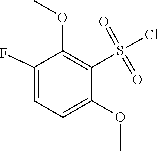

To a solution of 2,6-dimethoxybenzene-1-sulfonyl chloride (2.50 g, 10.6 mmol) in nitromethane (30 mL) was added N-fluoro-N′-chloromethyltriethylenediamine bis(tetrafluoro-borate) (3.37 g, 9.51 mmol). The reaction mixture was stirred at 75° C. overnight. The reaction mixture was diluted with brine (50 mL) and EtOAc (50 mL). The aqueous layer was extracted with EtOAc (3×20 mL). The combined organic layers were washed with brine, dried over MgSO₄, filtered, and concentrated. The crude material was purified by silica gel chromatography (0-40% EtOAc/hexanes) to afford the title compound (1.87 g, 7.33 mmol, 69.4% yield) as a tan oil. ¹H NMR (400 MHz, CDCl₃) δ 7.41 (dd, J=10.6, 9.4 Hz, 1H), 6.76 (dd, J=9.4, 3.5 Hz, 1H), 4.11 (d, J=2.2 Hz, 3H), 3.99 (s, 3H).

### Step 2. 3-Bromo-5-fluoro-2,6-dimethoxybenzenesulfonyl chloride

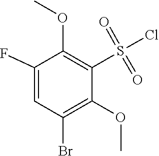 [SMILES: COc1c(F)cc(Br)c(OC)c1S(=O)(=O)Cl]

A solution of 3-fluoro-2,6-dimethoxybenzenesulfonyl chloride (1.83 g, 7.19 mmol) and N-bromosuccinimide (1.54 mg, 8.63 mmol) in nitromethane (12 mL) was stirred at 95° C. for 18 h. The reaction mixture was cooled to room temperature and diluted with water (25 mL) and EtOAc (25 mL). The aqueous layer was extracted with EtOAc (3×15 mL). The combined organic layers were washed with brine, dried over MgSO₄, filtered, and concentrated. The crude material was purified by silica gel chromatography (0-20% EtOAc/hexanes) to afford the title compound (2.17 g, 6.51 mmol, 90.4% yield) as a yellow oil. ¹H NMR (400 MHz, CDCl₃) δ 7.71 (d, J=10.3 Hz, 1H), 4.13 (d, J=2.3 Hz, 3H), 4.01 (s, 3H).

### Step 3. N-(6-((1H-Pyrazol-1-yl)methyl)-4-methoxybenzo[d]isoxazol-3-yl)-3-bromo-5-fluoro-2,6-dimethoxybenzenesulfonamide

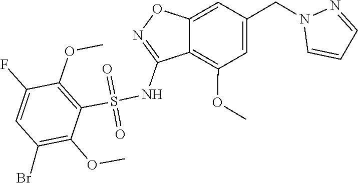 [SMILES: COc1c(F)cc(Br)c(OC)c1S(=O)(=O)Nc1noc2cc(Cn3cccn3)cc(OC)c12]

The title compound was synthesized according to procedures analogous to Example 28, Step 3. LCMS calc. for C₂₀H₁₉BrFN₄O₆S [M+H]⁺: m/z=541.0, 543.0; Found: 541.0, 543.1.

### Step 4. 7-(((tert-Butyldiphenylsilyl)oxy)methyl)-2-azaspiro[3.5]nonane

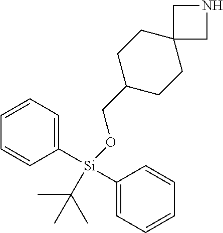 [SMILES: CC(C)(C)[Si](OCC1CCC2(CC1)CNC2)(c1ccccc1)c1ccccc1]

The title compound was synthesized according to procedures analogous to Example 35, Steps 1-2, using tert-butyl 7-(hydroxymethyl)-2-azaspiro[3.5]nonane-2-carboxylate. LCMS calc. for C₂₅H₃₆NOSi [M+H]⁺: m/z=394.3; Found: 394.3.

### Step 5. N-(6-((1H-Pyrazol-1-yl)methyl)-4-methoxybenzo[d]isoxazol-3-yl)-3-(7-(((tert-butyldiphenylsilyl)oxy)methyl)-2-azaspiro[3.5]nonan-2-yl)-5-fluoro-2,6-dimethoxybenzenesulfonamide

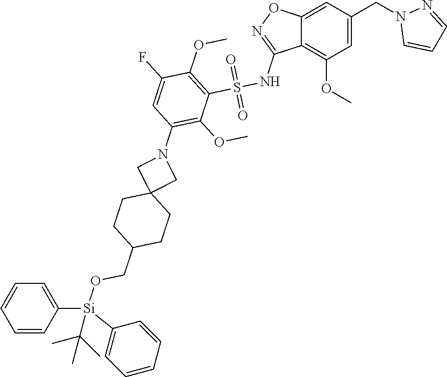 [SMILES: COc1c(F)cc(N2CC3(CCC(CO[Si](c4ccccc4)(c4ccccc4)C(C)(C)C)CC3)C2)c(OC)c1S(=O)(=O)Nc1noc2cc(Cn3cccn3)cc(OC)c12]

To a nitrogen sparged solution of 2-azaspiro[3.5]nonan-7-ylmethoxy-tert-butyl-diphenylsilane (291 mg, 0.739 mmol), N-(6-((1H-pyrazol-1-yl)methyl)-4-methoxybenzo[d]-isoxazol-3-yl)-3-bromo-5-fluoro-2,6-dimethoxybenzenesulfonamide (200 mg, 0.369 mmol), and cesium carbonate (602 mg, 1.85 mmol) in 1,4-dioxane (4 mL) was added Pd-PEPPSI-iHeptCl (CAS: 1814936-54-3, 36.0 mg, 0.0369 mmol). The reaction mixture was sparged with nitrogen an additional 5 min then stirred at 100° C. in a sealed vial for 2 h. The reaction was diluted with water (10 mL) and DCM (10 mL) The layers were separated, and the aqueous layer was extracted with DCM (3×10 mL). The combined organic layers were washed with brine, dried over MgSO₄, and filtered. The crude material was concentrated onto Celite and purified by silica gel chromatography (20-90% EtOAc/hexanes) to afford the title compound (147 mg, 0.172 mmol, 46.6% yield) as a tan solid. LCMS calc. for C₄₅H₅₃FN₅O₇SSi [M+H]⁺: m/z=854.3; Found: 854.2.

### Step 6. N-(6-((1H-Pyrazol-1-yl)methyl)-4-methoxybenzo[d]isoxazol-3-yl)-3-(7-((4-(4-(2,6-dioxopiperidin-3-yl)phenyl)piperidin-1-yl)methyl)-2-azaspiro[3.5]nonan-2-yl)-5-fluoro-2, 6-dimethoxybenzenesulfonamide

The title compound was synthesized according to procedures analogous to Example 35, Steps 3-5 using 3-(4-(piperidin-4-yl)phenyl)piperidine-2,6-dione (which can be prepared as described in WO2023125907A1). LCMS calc. for C₄₅H₅₃FN₇O₈S [M+H]⁺: m/z=870.4; Found: 870.4.

### Example 56. N-(6-((1H-Pyrazol-1-yl)methyl)-4-methoxybenzo[d]isoxazol-3-yl)-3-(7-((1-(3-(2,6-dioxopiperidin-3-yl)-1-methyl-1H-indazol-6-yl)piperidin-4-yl)methyl)-2,7-diazaspiro[3.5]nonan-2-yl)benzenesulfonamide

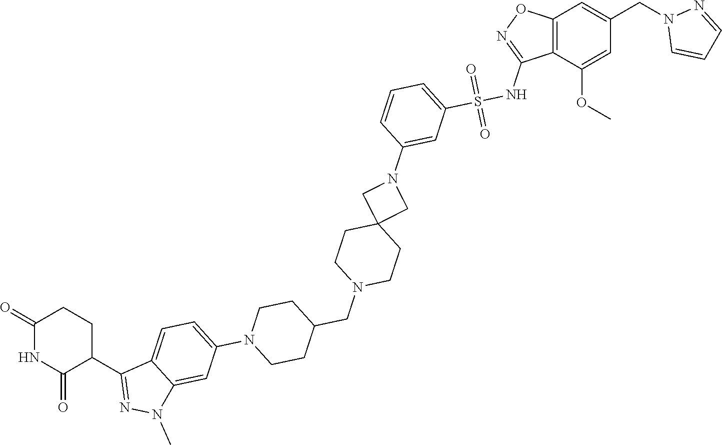 [SMILES: COc1cc(Cn2cccn2)cc2onc(NS(=O)(=O)c3cccc(N4CC5(CCN(CC6CCN(c7ccc8c(C9CCC(=O)NC9=O)nn(C)c8c7)CC6)CC5)C4)c3)c12]

The title compound was synthesized according to procedures analogous to Example 1, Step 6 using 1-(3-(2,6-dioxopiperidin-3-yl)-1-methyl-1H-indazol-6-yl)piperidine-4-carbaldehyde (which can be prepared as described in WO2024039901A₂). ¹H NMR (400 MHz, CD₃OD) δ 7.78 (d, J=2.3 Hz, 1H), 7.62 (d, J=9.0 Hz, 1H), 7.58 (d, J=2.0 Hz, 1H), 7.43-7.34 (m, 2H), 7.15 (d, J=2.0 Hz, 1H), 7.04 (d, J=9.1 Hz, 1H), 6.98 (s, 1H), 6.78 (s, 1H), 6.76-6.69 (m, 1H), 6.65 (s, 1H), 6.39 (t, J=2.2 Hz, 1H), 5.48 (s, 2H), 4.33 (dd, J=9.1, 5.1 Hz, 1H), 3.98 (d, J=1.5 Hz, 3H), 3.95-3.85 (m, 5H), 3.81 (s, 2H), 3.72 (s, 2H), 3.64 (d, J=12.7 Hz, 2H), 3.21-3.07 (m, 4H), 2.97 (t, J=12.2 Hz, 2H), 2.85-2.70 (m, 2H), 2.53-2.40 (m, 1H), 2.39-2.23 (m, 3H), 2.21-2.04 (m, 3H), 2.00 (d, J=12.7 Hz, 2H), 1.60 (q, J=11.9 Hz, 2H). LCMS calc. for C₄₄H₅₁N₁₀O₆S [M+H]⁺: m/z=847.4; Found: 847.6.

### Example 57. N-(6-((1H-Pyrazol-1-yl)methyl)-4-methoxybenzo[d]isoxazol-3-yl)-3-(7-((1-(3-(2,4-dioxotetrahydropyrimidin-1(2H)-yl)-1-methyl-1H-pyrazolo[3,4-b]pyridin-6-yl)piperidin-4-yl)methyl)-2,7-diazaspiro[3.5]nonan-2-yl)-2,6-dimethoxybenzene-sulfonamide

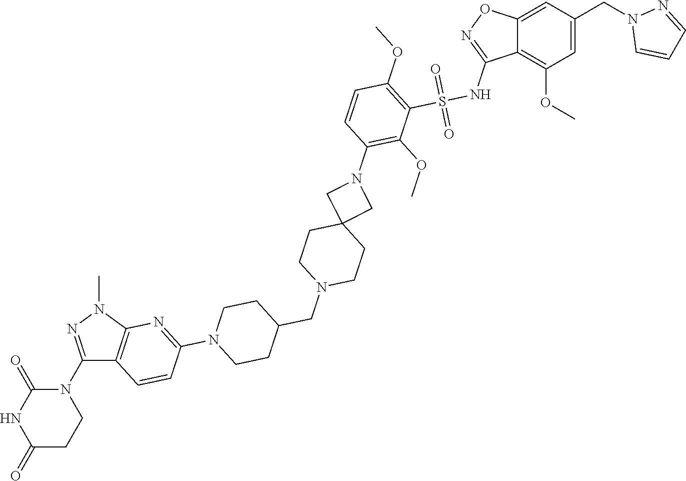 [SMILES: COc1ccc(N2CC3(CCN(CC4CCN(c5ccc6c(N7CCC(=O)NC7=O)nn(C)c6n5)CC4)CC3)C2)c(OC)c1S(=O)(=O)Nc1noc2cc(Cn3cccn3)cc(OC)c12]

### Step 1. 6-Bromo-3-iodo-1-methyl-1H-pyrazolo[3,4-b]pyridine

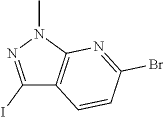

To a mixture of 6-bromo-1-methylpyrazolo[5,4-b]pyridine (5.00 g, 23.6 mmol) and N-iodosuccinimide (5.84 g, 25.9 mmol) in MeCN (117 mL) was added fluoroboric acid (50% aq., 14.8 mL, 120 mmol). The reaction mixture was stirred at 80° C. for 2 h. The mixture was cooled to room temperature, water was added, and the mixture was stirred rapidly for 5 min. The resulting solid was collected by filtration to afford the title compound (8.39 g, 24.8 mmol, quantitative yield) as an off-white solid. LCMS calc. for C₇H₆BrIN₃ [M+H]⁺: m/z=337.9, 339.9; Found: 337.7, 339.7.

### Step 2. Methyl 3-((6-bromo-1-methyl-1H-pyrazolo[3,4-b]pyridin-3-yl)amino)-propanoate

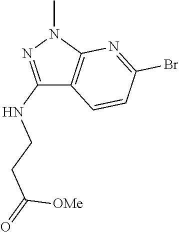 [SMILES: COC(=O)CCNc1nn(C)c2nc(Br)ccc12]

A nitrogen sparged solution of 6-bromo-3-iodo-1-methylpyrazolo[5,4-b]pyridine (8.39 g, 24.8 mmol), beta-alanine methyl ester, HCl (6.93 g, 49.7 mmol), copper(I) iodide (473 mg, 2.48 mmol), L-proline (572 mg, 4.97 mmol), and potassium phosphate tribasic (21.1 g, 99.4 mmol) in DMSO (83 mL) was stirred at 100° C. for 1.5 hr. The reaction was cooled to room temperature and diluted with water. The layers were separated, and the aqueous layer was extracted with DCM (3×). The combined organic layers were washed with water, dried over MgSO₄, filtered, and concentrated. The crude material was purified by silica gel chromatography (0-55% EtOAc/hexanes) to afford the title compound (715 mg, 2.28 mmol, 9.19% yield) as a yellow solid. LCMS calc. for C₁₁H₁₄BrN₄O₂ [M+H]⁺: m/z=313.0, 315.0; Found: 313.0, 315.0.

### Step 3. Methyl 3-(1-(6-bromo-1-methyl-1H-pyrazolo[3,4-b]pyridin-3-yl)ureido)-propanoate

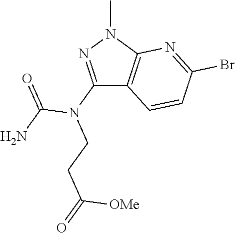 [SMILES: COC(=O)CCN(C(N)=O)c1nn(C)c2nc(Br)ccc12]

A solution of methyl 3-[(6-bromo-1-methylpyrazolo[3,4-b]pyridin-3-yl)amino]-propanoate (715 mg, 2.28 mmol) and sodium cyanate (445 mg, 6.85 mmol) in acetic acid (15 mL) was stirred for 5.5 h. The mixture was neutralized with sat. NaHCO₃ (aq.) and extracted with DCM (3×). The combined organic layers were washed with water, dried over MgSO₄, filtered, and concentrated. The material was carried forward without further purification. LCMS calc. for C₁₂H₁₅BrN₅O₃ [M+H]⁺: m/z=356.0, 358.0; Found: 355.9, 357.9.

### Step 4. 1-(6-Chloro-1-methyl-1H-pyrazolo[3,4-b]pyridin-3-yl)dihydropyrimidine-2,4(1H,3H)-dione

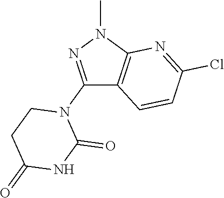 [SMILES: Cn1nc(N2CCC(=O)NC2=O)c2ccc(Cl)nc21]

A solution of methyl 3-[(6-bromo-1-methylpyrazolo[5,4-b]pyridin-3-yl)-carbamoyl-amino]propanoate (813 mg, 2.28 mmol) in HCl (37% aq., 20.5 mL, 245 mmol) was stirred at 50° C. for 3 hr. The reaction was cooled to room temperature and neutralized with sat. NaHCO₃ (aq.), and extracted with DCM (×1) and 3:1 (v/v) CHCl₃/IPA (×3). The combined organic layers were washed with water, dried over MgSO₄, filtered, and concentrated to afford the title compound (400 mg, 1.43 mmol, 62.7% yield over 2 steps). LCMS calc. for C₁₁H₁₁ClN₅O₂[M+H]⁺: m/z=280.1; Found: 280.1.

### Step 5. 1-(3-(2,4-Dioxotetrahydropyrimidin-1(2H)-yl)-1-methyl-1H-pyrazolo[3,4-b]pyridin-6-yl)piperidine-4-carbaldehyde

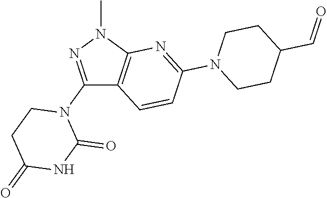 [SMILES: Cn1nc(N2CCC(=O)NC2=O)c2ccc(N3CCC(C=O)CC3)nc21]

To a mixture of 1-(6-chloro-1-methylpyrazolo[5,4-b]pyridin-3-yl)-1,3-diazinane-2,4-dione (335 mg, 1.20 mmol) and 4-(dimethoxymethyl)piperidine (381 mg, 2.40 mmol) in DMF (12 mL) was added diisopropylethyl amine (0.626 mL, 3.59 mmol). The reaction mixture was stirred at 70° C. overnight. The reaction was cooled to room temperature and diluted with water. The layers were separated, and the aqueous layer was extracted with DCM (3×). The combined organic layers were washed with water, dried over MgSO₄, filtered, and concentrated. crude material was purified by prep-HPLC on a C18 column (25.5-45.5% CH₃CN/0.2% TFA (aq.)) to afford the title compound (20.0 mg, 0.0561 mmol, 4.69% yield) (acetal deprotected under purification conditions). LCMS calc. for C₁₇H₂₁N₆O₃ [M+H]⁺: m/z=357.2; Found: 357.2.

### Step 6. N-(6-((1H-Pyrazol-1-yl)methyl)-4-methoxybenzo[d]isoxazol-3-yl)-3-(7-((1-(3-(2,4-dioxotetrahydropyrimidin-1(2H)-yl)-1-methyl-1H-pyrazolo[3,4-b]pyridin-6-yl)piperidin-4-yl)methyl)-2, 7-diazaspiro[3.5]nonan-2-yl)-2,6-dimethoxybenzenesulfonamide

The title compound was synthesized according to procedures analogous to Example 1, Step 6 using N-(6-((1H-pyrazol-1-yl)methyl)-4-methoxybenzo[d]isoxazol-3-yl)-2,6-dimethoxy-3-(2,7-diazaspiro[3.5]nonan-2-yl)benzenesulfonamide (which can be prepared as described in WO2023245150A1) and 1-(3-(2,4-dioxotetrahydropyrimidin-1(2H)-yl)-1-methyl-1H-pyrazolo[3,4-b]pyridin-6-yl)piperidine-4-carbaldehyde. LCMS calc. for C₄₄H₅₃N₁₂O₈S [M+H]⁺: m/z=909.4; Found: 909.4.

### Example 58. N-(6-((1H-Pyrazol-1-yl)methyl)-4-methoxybenzo[d]isoxazol-3-yl)-3-(7-((1-(3-(2,6-dioxopiperidin-3-yl)-1-methyl-1H-pyrazolo[3,4-b]pyridin-6-yl)piperidin-4-yl)methyl)-2,7-diazaspiro[3.5]nonan-2-yl)-5-fluoro-2,6-dimethoxybenzenesulfonamide

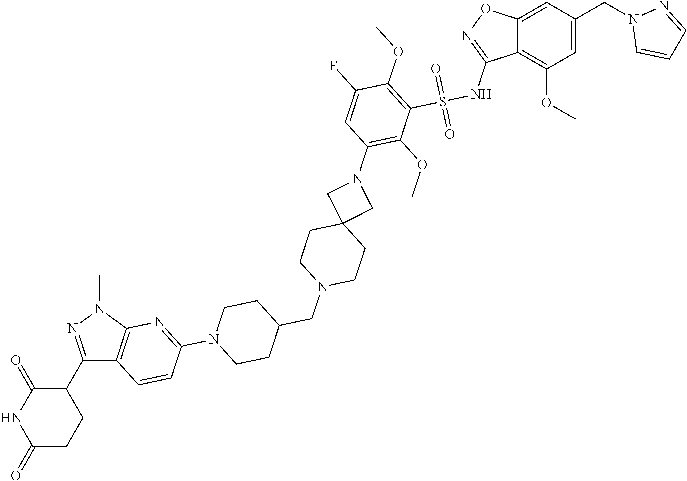 [SMILES: COc1c(F)cc(N2CC3(CCN(CC4CCN(c5ccc6c(C7CCC(=O)NC7=O)nn(C)c6n5)CC4)CC3)C2)c(OC)c1S(=O)(=O)Nc1noc2cc(Cn3cccn3)cc(OC)c12]

### Step 1. 3-(2,6-Bis(benzyloxy)pyridin-3-yl)-6-bromo-1-methyl-1H-pyrazolo-[3,4-b]pyridine

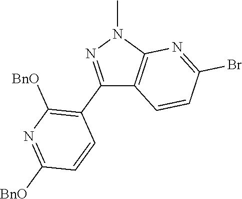 [SMILES: Cn1nc(-c2ccc(OCc3ccccc3)nc2OCc2ccccc2)c2ccc(Br)nc21]

A mixture of 2,6-bis(phenylmethoxy)-3-(4,4,5,5-tetramethyl-1,3,2-dioxaborolan-2-yl)-pyridine (720 mg, 1.73 mmol), 6-bromo-3-iodo-1-methylpyrazolo[5,4-b]pyridine (417 mg, 1.23 mmol) (From Example 57, Step 1), [1,1′-bis(diphenylphosphino)ferrocene]dichloro-palladium(II) dichloromethane (101 mg, 0.123 mmol), and potassium carbonate (511 mg, 3.70 mmol) in 1,4-dioxane (8 mL) and water (2 mL) was sparged with nitrogen for −1 min then stirred at 80° C. for 1.75 h. The reaction was cooled to room temperature and concentrated. The crude material was purified by silica gel chromatography (0-30% EtOAc/hexanes) to afford the title compound (312 mg, 0.623 mmol, 50.5% yield) as a white solid. LCMS calc. for C₂₆H₂₂BrN₄O₂ [M+H]⁺: m/z=501.1, 503.1; Found: 501.0, 503.0.

### Step 2. 3-(2,6-Bis(benzyloxy)pyridin-3-yl)-6-(4-(dimethoxymethyl)piperidin-1-yl)-1-methyl-1H-pyrazolo[3,4-b]pyridine

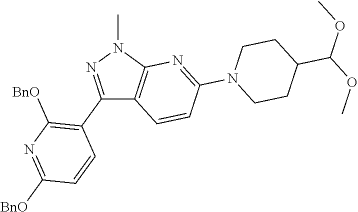 [SMILES: COC(OC)C1CCN(c2ccc3c(-c4ccc(OCc5ccccc5)nc4OCc4ccccc4)nn(C)c3n2)CC1]

A mixture of 2-(dicyclohexylphosphino)-2′,4′,6′-tri-i-propyl-1,1′-biphenyl (XPhos) (232 mg, 0.486 mmol), 3-[2,6-bis(phenylmethoxy)-3-pyridinyl]-6-bromo-1-methylpyrazolo-[3,4-b]pyridine (1220 mg, 2.43 mmol), 4-(dimethoxymethyl)piperidine (547 mg, 3.44 mmol), cesium carbonate (2650 mg, 8.13 mmol) and tris(dibenzylideneacetone)dipalladium (0) (223 mg, 0.243 mmol) in 1,4-dioxane (24 mL) was sparged with nitrogen for ~3 min, then stirred at 110° C. overnight. The reaction mixture was concentrated and purified by silica gel chromatography (0-50% EtOAc/hexanes) to afford the title compound (439 mg, 0.757 mmol, 31.1% yield) as an orange oil. LCMS calc. for C₃₄H₃₈N₅O₄ [M+H]⁺: m/z=580.3; Found: 580.3.

### Step 3. 1-(3-(2,6-Dioxopiperidin-3-yl)-1-methyl-1H-pyrazolo[3,4-b]pyridin-6-yl)piperidine-4-carbaldehyde

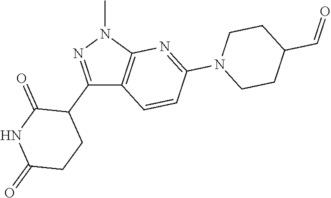 [SMILES: Cn1nc(C2CCC(=O)NC2=O)c2ccc(N3CCC(C=O)CC3)nc21]

The title compound was synthesized according to procedures analogous to Example 4, Steps 3-4. LCMS calc. for C₁₈H₂₂N₅O₃ [M+H]⁺: m/z=356.2; Found: 356.1.

### Step 4. N-(6-((1H-Pyrazol-1-yl)methyl)-4-methoxybenzo[d]isoxazol-3-yl)-3-fluoro-2,6-dimethoxy-5-(2, 7-diazaspiro[3.5]nonan-2-yl)benzenesulfonamide

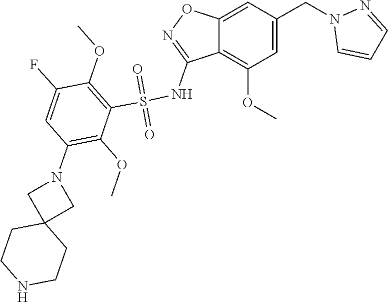 [SMILES: COc1c(F)cc(N2CC3(CCNCC3)C2)c(OC)c1S(=O)(=O)Nc1noc2cc(Cn3cccn3)cc(OC)c12]

The title compound was synthesized according to procedures analogous to Example 12, Step 2 using N-(6-((1H-pyrazol-1-yl)methyl)-4-methoxybenzo[d]isoxazol-3-yl)-3-bromo-5-fluoro-2,6-dimethoxybenzenesulfonamide (From Example 55, Step 3). LCMS calc. for C₂₇H₃₂FN₆O₆S [M+H]⁺: m/z=587.2; Found: 587.2.

### Step 5. N-(6-((1H-Pyrazol-1-yl)methyl)-4-methoxybenzo[d]isoxazol-3-yl)-3-(7-((1-(3-(2,6-dioxopiperidin-3-yl)-1-methyl-1H-pyrazolo[3,4-b]pyridin-6-yl)piperidin-4-yl)methyl)-2, 7-diazaspiro[3.5]nonan-2-yl)-5-fluoro-2,6-dimethoxybenzenesulfonamide

The title compound was synthesized according to procedures analogous to Example 1, Step 6. LCMS calc. for C₄₅H₅₃FN₁₁O₈S [M+H]⁺: m/z=926.4; Found: 926.1.

### Example 59. N-(6-((1H-Pyrazol-1-yl)methyl)-4-methoxybenzo[d]isoxazol-3-yl)-3-(7-((1-(3-(2,4-dioxotetrahydropyrimidin-1(2H)-yl)-7-fluoro-1-methyl-1H-indazol-6-yl)piperidin-4-yl)methyl)-2,7-diazaspiro[3.5]nonan-2-yl)-5-fluoro-2,6-dimethoxybenzene-sulfonamide

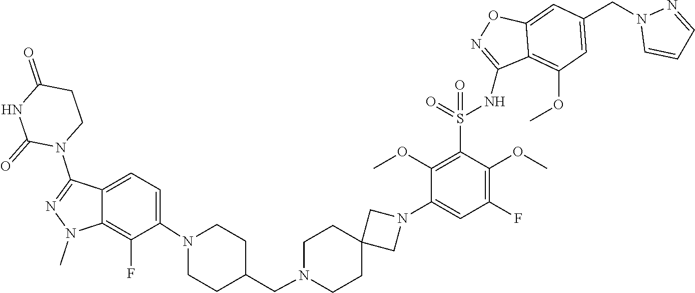 [SMILES: COc1c(F)cc(N2CC3(CCN(CC4CCN(c5ccc6c(N7CCC(=O)NC7=O)nn(C)c6c5F)CC4)CC3)C2)c(OC)c1S(=O)(=O)Nc1noc2cc(Cn3cccn3)cc(OC)c12]

### Step 1. 1-(6-(4-(Dimethoxymethyl)piperidin-1-yl)-7-fluoro-1-methyl-1H-indazol-3-yl)dihydropyrimidine-2,4(1H,3H)-dione

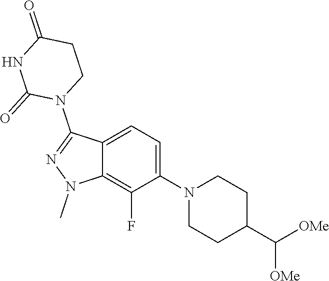 [SMILES: COC(OC)C1CCN(c2ccc3c(N4CCC(=O)NC4=O)nn(C)c3c2F)CC1]

To a solution of 1-(6-bromo-7-fluoro-1-methyl-indazol-3-yl)hexahydropyrimidine-2,4-dione (50.0 mg, 0.147 mmol), 4-(dimethoxymethyl)piperidine (46.7 mg, 0.293 mmol), and RuPhos Pd G3 (CAS: 1445085-77-7, 24.5 mg, 0.0293 mmol) was added cesium carbonate (191 mg, 0.586 mmol). The reaction was sparged with nitrogen and stirred for 4 h at 100° C. The reaction was cooled to room temperature and concentrated. The crude material was purified by silica gel chromatography (0-75% EtOAc/hexanes) to afford the title compound (16 mg, 0.028 mmol, 19% yield) as an orange solid. C₂₀H₂₇FN₅O₄ [M+H]⁺: m/z=420.2; Found: 420.2.

### Step 2. 1-(3-(2,4-Dioxotetrahydropyrimidin-1(2H)-yl)-7-fluoro-1-methyl-1H-indazol-6-yl)piperidine-4-carbaldehyde

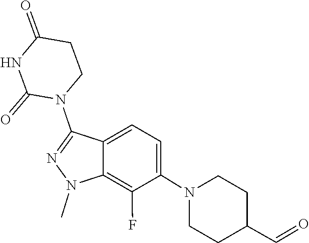 [SMILES: Cn1nc(N2CCC(=O)NC2=O)c2ccc(N3CCC(C=O)CC3)c(F)c21]

The title compound was synthesized according to procedures analogous to Example 4, Step 4. LCMS calc. for C₁₈H₂₁FN₅O₃[M+H]⁺: m/z=374.2; Found: 374.1.

### Step 3. N-(6-((1H-Pyrazol-1-yl)methyl)-4-methoxybenzo[d]isoxazol-3-yl)-3-(7-((1-(3-(2,4-dioxotetrahydropyrimidin-1(2H)-yl)-7-fluoro-1-methyl-1H-indazol-6-yl)piperidin-4-yl)methyl)-2,7-diazaspiro[3.5]nonan-2-yl)-5-fluoro-2,6-dimethoxybenzenesulfonamide

The title compound was synthesized according to procedures analogous to Example 1, Step 6, using N-(6-((1H-pyrazol-1-yl)methyl)-4-methoxybenzo[d]isoxazol-3-yl)-3-fluoro-2,6-dimethoxy-5-(2,7-diazaspiro[3.5]nonan-2-yl)benzenesulfonamide (From Example 58, Step 4). LCMS calc. for C₄₅H₅₂F₂N₁₁O₈S [M+H]⁺: m/z=944.4; Found: 944.3.

### Example 60. N-(6-((1H-Pyrazol-1-yl)methyl)-4-methoxybenzo[d]isoxazol-3-yl)-3-(4-((7-(3-(2,4-dioxotetrahydropyrimidin-1(2H)-yl)-1-methyl-1H-indazol-6-yl)-7-azaspiro[3.5]-nonan-2-yl)methyl)piperazin-1-yl)-5-fluoro-2,6-dimethoxybenzenesulfonamide

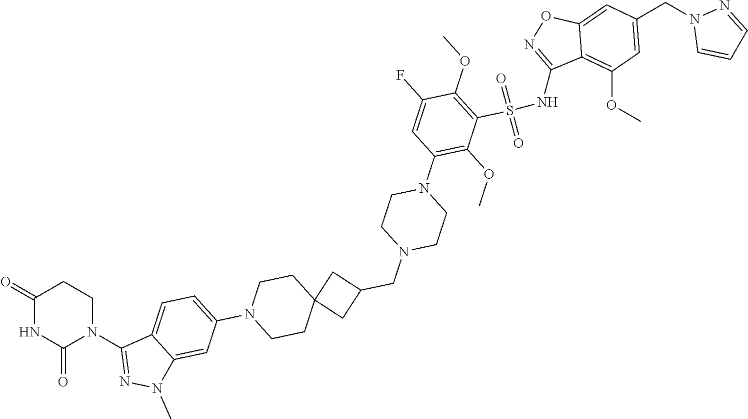 [SMILES: COc1c(F)cc(N2CCN(CC3CC4(CCN(c5ccc6c(N7CCC(=O)NC7=O)nn(C)c6c5)CC4)C3)CC2)c(OC)c1S(=O)(=O)Nc1noc2cc(Cn3cccn3)cc(OC)c12]

### Step 1. 1-(6-(2-(Dimethoxymethyl)-7-azaspiro[3.5]nonan-7-yl)-1-methyl-1H-indazol-3-yl)dihydropyrimidine-2,4(1H,3H)-dione

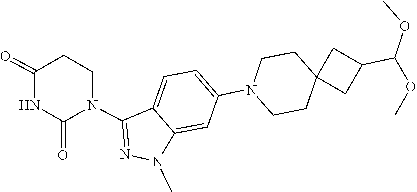 [SMILES: COC(OC)C1CC2(CCN(c3ccc4c(N5CCC(=O)NC5=O)nn(C)c4c3)CC2)C1]

The title compound was synthesized according to procedures analogous to Example 59, Step 1, using 1-(6-bromo-1-methyl-1H-indazol-3-yl)dihydropyrimidine-2,4(1H,3H)-dione (which can be prepared as described in WO2023059792A1) and 2-(dimethoxymethyl)-7-azaspiro[3.5]nonane (which can be prepared as described in WO20244015408A1). LCMS calc. for C₂₃H₃₂N₅O₄ [M+H]⁺: m/z=442.2; Found: 442.3.

### Step 2. 7-(3-(2,4-Dioxotetrahydropyrimidin-1(2H)-yl)-1-methyl-1H-indazol-6-yl)-7-azaspiro[3.5]nonane-2-carbaldehyde

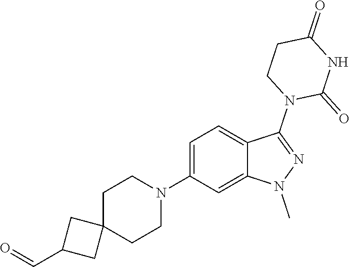 [SMILES: Cn1nc(N2CCC(=O)NC2=O)c2ccc(N3CCC4(CC3)CC(C=O)C4)cc21]

The title compound was synthesized according to procedures analogous to Example 4, Step 4. LCMS calc. for C₂₁H₂₆N₅O₃ [M+H]⁺: m/z=396.2; Found: 396.2.

### Step 3. N-(6-((1H-Pyrazol-1-yl)methyl)-4-methoxybenzo[d]isoxazol-3-yl)-3-fluoro-2,6-dimethoxy-5-(piperazin-1-yl)benzenesulfonamide

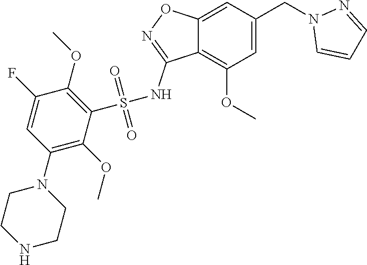 [SMILES: COc1c(F)cc(N2CCNCC2)c(OC)c1S(=O)(=O)Nc1noc2cc(Cn3cccn3)cc(OC)c12]

To a nitrogen sparged solution of N-(6-((1H-pyrazol-1-yl)methyl)-4-methoxybenzo-[d]isoxazol-3-yl)-3-bromo-5-fluoro-2,6-dimethoxybenzenesulfonamide (100 mg, 0.185 mmol, from Example 55, Step 3), tert-butyl 1-piperazinecarboxylate (68.8 mg, 0.369 mmol), cesium carbonate (181 mg, 0.554 mmol) in 1,4-dioxane (1.8 mL) was added Pd-PEPPSI-iHeptCl (CAS: 1158652-41-5, 18.0 mg, 0.0185 mmol). The reaction was stirred under a nitrogen atmosphere for 48 h at 100° C. The reaction mixture was cooled to room temperature and concentrated. TFA (200 μL) was added, and the reaction mixture was stirred for 1 h then concentrated. The crude material was purified by silica gel chromatography (0-20% MeOH/DCM) to afford the title compound (80 mg, 0.15 mmol, 79% yield) as a yellow solid. LCMS calc. for C₂₄H₂₈FN₆O₆S [M+H]⁺: m/z=547.2; Found: 547.2.

### Step 4. N-(6-((1H-Pyrazol-1-yl)methyl)-4-methoxybenzo[d]isoxazol-3-yl)-3-(4-((7-(3-(2,4-dioxotetrahydropyrimidin-1(2H)-yl)-1-methyl-1H-indazol-6-yl)-7-azaspiro[3.5]-nonan-2-yl)methyl)piperazin-1-yl)-5-fluoro-2,6-dimethoxybenzenesulfonamide

The title compound was synthesized according to procedures analogous to Example 1, Step 6. LCMS calc. for C₄₅H₅₃FN₁₁O₈S [M+H]⁺: m/z=926.4; Found: 926.4.

### Example 61. N-(6-((1H-Pyrazol-1-yl)methyl)-4-methoxybenzo[d]isoxazol-3-yl)-3-(6-((1-(3-(2,6-dioxopiperidin-3-yl)-1-methyl-1H-indazol-6-yl)piperidin-4-yl)methyl)-5,6,7,8-tetrahydro-1,6-naphthyridin-2-yl)-5-fluoro-2,6-dimethoxybenzenesulfonamide

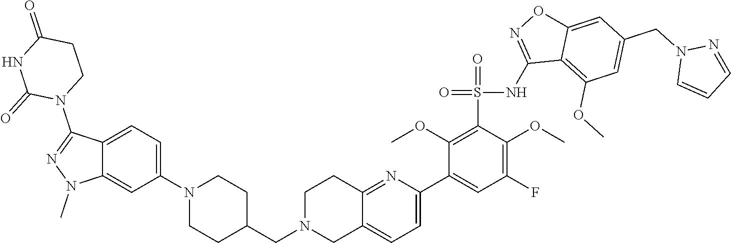 [SMILES: COc1c(F)cc(-c2ccc3c(n2)CCN(CC2CCN(c4ccc5c(N6CCC(=O)NC6=O)nn(C)c5c4)CC2)C3)c(OC)c1S(=O)(=O)Nc1noc2cc(Cn3cccn3)cc(OC)c12]

Step 1: N-(6-((1H-Pyrazol-1-yl)methyl)-4-methoxybenzo[d]isoxazol-3-yl)-3-bromo-5-fluoro-2,6-dimethoxy-N-((2-(trimethylsilyl)ethoxy)methyl)benzenesulfonamide

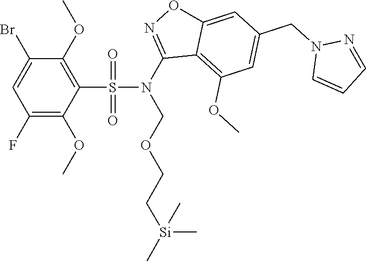 [SMILES: COc1c(F)cc(Br)c(OC)c1S(=O)(=O)N(COCC[Si](C)(C)C)c1noc2cc(Cn3cccn3)cc(OC)c12]

A solution of N-(6-((1H-pyrazol-1-yl)methyl)-4-methoxybenzo[d]isoxazol-3-yl)-3-bromo-5-fluoro-2,6-dimethoxybenzenesulfonamide (300 mg, 0.554 mmol, Example 55, Step 3) and sodium hydride (88.7 mg, 2.22 mmol) in THF (10 mL) was stirred for 5 min. (2-(chloromethoxy)ethyl)trimethylsilane (393 μL, 2.22 mmol) was added. The mixture was stirred at room temperature for 2 h and then at 40° C. overnight. The reaction was quenched with slow addition of brine (30 mL). The mixture was then extracted with DCM (3×40 mL), dried over anhydrous MgSO₄, filtered, and concentrated. The crude material was purified by silica gel chromatography (0-10% MeOH/DCM) to afford the title compound (353 mg, 94.8% yield) as light-yellow gum which solidified slowly. LCMS calc. for C₂₆H₃₃BrFN₄O₇SSi [M+H]⁺: m/z=671.1, 673.1; Found: 671.0, 673.0.

### Step 2: N-(6-((1H-Pyrazol-1-yl)methyl)-4-methoxybenzo[d]isoxazol-3-yl)-3-fluoro-2,6-dimethoxy-5-(4,4,5,5-tetramethyl-1,3,2-dioxaborolan-2-yl)-N-((2-(trimethylsilyl)ethoxy)-methyl)benzenesulfonamide

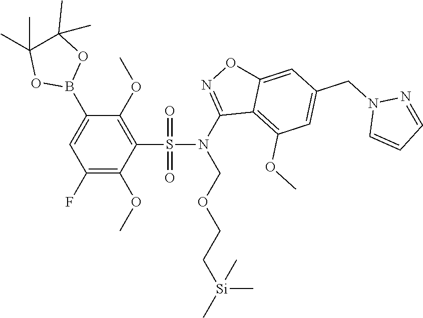 [SMILES: COc1c(F)cc(B2OC(C)(C)C(C)(C)O2)c(OC)c1S(=O)(=O)N(COCC[Si](C)(C)C)c1noc2cc(Cn3cccn3)cc(OC)c12]

A solution of N-(6-((1H-pyrazol-1-yl)methyl)-4-methoxybenzo[d]isoxazol-3-yl)-3-bromo-5-fluoro-2,6-dimethoxy-N-((2-(trimethylsilyl)ethoxy)methyl)benzene-sulfonamide (50 mg, 0.074 mmol) and 2-isopropoxy-4,4,5,5-tetramethyl-1,3,2-dioxaborolane (61 μL, 0.30 mmol) in THF (2 mL) was stirred under a nitrogen atmosphere at 0° C. A solution of isopropylmagnesium chloride lithium chloride complex (286 μL, 0.372 mmol, 1.3 M in THF) was added drop wise. The mixture was stirred at room temperature for 20 minutes, then quenched with saturated NH₄Cl (aq.) (20 mL) and brine (20 mL). The mixture was extracted with ethyl acetate (3×30 mL), dried over anhydrous MgSO₄, filtered, and concentrated. The crude material was purified by silica gel chromatography (0-10% MeOH/DCM) to afford the title compound (56 mg, quantitative yield) as colorless oil. LCMS calc. for C₃₂H₄₅BFN₄O₉SSi [M+H]⁺: m/z=719.3; Found: 719.2.

### Step 3: N-(6-((1H-Pyrazol-1-yl)methyl)-4-methoxybenzo[d]isoxazol-3-yl)-3-fluoro-2,6-dimethoxy-5-(5, 6, 7,8-tetrahydro-1,6-naphthyridin-2-yl)benzenesulfonamide

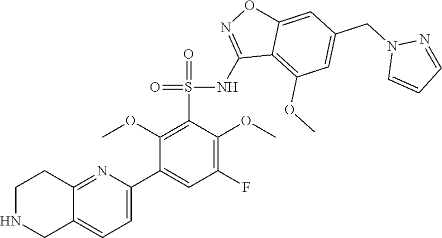 [SMILES: COc1c(F)cc(-c2ccc3c(n2)CCNC3)c(OC)c1S(=O)(=O)Nc1noc2cc(Cn3cccn3)cc(OC)c12]

A solution of N-(6-((1H-pyrazol-1-yl)methyl)-4-methoxybenzo[d]isoxazol-3-yl)-3-fluoro-2,6-dimethoxy-5-(4,4,5,5-tetramethyl-1,3,2-dioxaborolan-2-yl)-N-((2-(trimethylsilyl)-ethoxy)methyl)benzenesulfonamide (53 mg, 0.074 mmol) in water (0.2 mL) and 1,4-dioxane (2 mL) was sparged with nitrogen for 3 min. Potassium carbonate (41 mg, 0.29 mmol), SPhos Pd G2 (CAS: 1375325-64-6) (11 mg, 0.015 mmol) and tert-butyl 2-chloro-7,8-dihydro-1,6-naphthyridine-6(5H)-carboxylate (40 mg, 0.15 mmol) were added. The mixture was stirred under a nitrogen atmosphere at 80° C. for 1 h, cooled to room temperature, diluted with a 6:1 (v/v) solution of DCM/MeOH (5 mL), and passed through a pad of silica gel eluting with 6:1 (v/v) DCM/MeOH (50 mL). The volatiles were removed under reduced pressure. The resulting material was stirred in DCM (1 mL) and 2,2,2-trifluoroacetic acid (0.56 mL, 7.4 mmol) at room temperature for 3 h. The mixture was diluted with MeCN (3 mL), partially concentrated, and purified by preparative HPLC on a C18 column (24.6-44.6% CH₃CN/0.2% TFA (aq.)) to afford the title compound as TFA salt (32 mg, 61% yield), a white solid. LCMS calc. for C₂₈H₂₈FN₆₀₆S [M+H]⁺: m/z=595.2; Found: 595.2.

### Step 4: N-(6-((1H-Pyrazol-1-yl)methyl)-4-methoxybenzo[d]isoxazol-3-yl)-3-(6-((1-(3-(2,6-dioxopiperidin-3-yl)-1-methyl-1H-indazol-6-yl)piperidin-4-yl)methyl)-5,6,7,8-tetrahydro-1,6-naphthyridin-2-yl)-5-fluoro-2,6-dimethoxybenzenesulfonamide

The title compound was synthesized by procedures analogous to those outlined in Example 2, using 1-(3-(2,6-dioxopiperidin-3-yl)-1-methyl-1H-indazol-6-yl)piperidine-4-carbaldehyde (which can be prepared as described in WO2024039901A₂). ¹H NMR (400 MHz, CD₃OD) δ 7.81-7.74 (m, 4H), 7.69 (dd, J=8.7, 0.8 Hz, 1H), 7.59 (dd, J=2.0, 0.7 Hz, 1H), 7.15-7.07 (m, 2H), 6.82-6.80 (m, 1H), 6.71 (s, 1H), 6.39 (t, J=2.2 Hz, 1H), 5.50 (s, 2H), 4.67 (br s, 2H), 4.35 (dd, J=9.3, 5.1 Hz, 1H), 4.07 (d, J=2.1 Hz, 3H), 4.0 (s, 3H), 3.98 (s, 3H), 3.94-3.75 (br m, 4H), 3.56 (s, 3H), 3.45-3.36 (m, 4H), 3.19-3.09 (m, 2H), 2.85-2.70 (m, 2H), 2.55-2.41 (m, 1H), 2.39-2.27 (m, 2H), 2.11 (br m, 2H), 1.70 (qd, J=12.3, 3.8 Hz, 2H). ¹⁹F NMR (376 MHz, MeOD) δ −77.5, −132.7. LCMS calc. for C₄₇H₅₀FN₁₀O₈S [M+H]⁺: m/z=933.3; Found: 932.9.

### Example 62. N-(6-((1H-Pyrazol-1-yl)methyl)-4-methoxybenzo[d]isoxazol-3-yl)-4′-((4-(3-(2,4-dioxotetrahydropyrimidin-1(2H)-yl)-1-methyl-1H-indazol-6-yl)piperidin-1-yl)methyl)-2,4-dimethoxy-3′-methyl-[1,1′-biphenyl]-3-sulfonamide

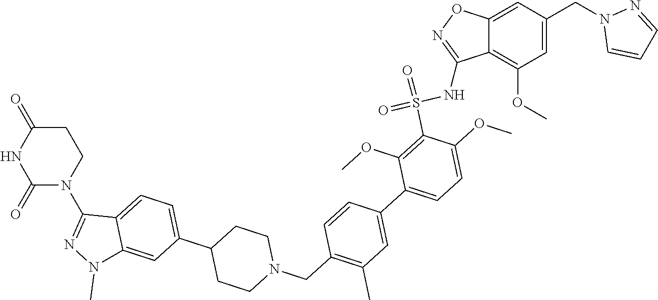 [SMILES: COc1ccc(-c2ccc(CN3CCC(c4ccc5c(N6CCC(=O)NC6=O)nn(C)c5c4)CC3)c(C)c2)c(OC)c1S(=O)(=O)Nc1noc2cc(Cn3cccn3)cc(OC)c12]

### Step 1: N-(6-((1H-Pyrazol-1-yl)methyl)-4-methoxybenzo[d]isoxazol-3-yl)-4′-formyl-2,4-dimethoxy-3′-methyl-[1,1′-biphenyl]-3-sulfonamide

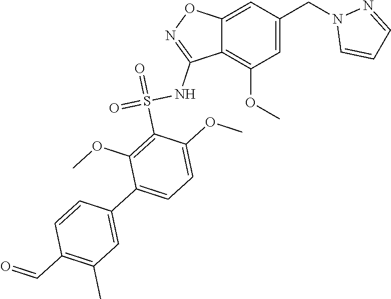 [SMILES: COc1ccc(-c2ccc(C=O)c(C)c2)c(OC)c1S(=O)(=O)Nc1noc2cc(Cn3cccn3)cc(OC)c12]

A solution of N-(6-((1H-pyrazol-1-yl)methyl)-4-methoxybenzo[d]isoxazol-3-yl)-3-bromo-2,6-dimethoxybenzenesulfonamide (which can be prepared as described in WO2023245150 A1) (30 mg, 0.057 mmol) in water (0.20 mL) and 1,4-dioxane (1 mL) was sparged with nitrogen for 3 min. Potassium carbonate (24 mg, 0.17 mmol), SPhos Pd G2 (CAS: 1375325-64-6) (4.1 mg, 0.0057 mmol) and (4-formyl-3-methylphenyl)boronic acid (24 mg, 0.14 mmol) were added. The mixture was stirred under a nitrogen atmosphere at 90° C. for 40 minutes. After cooling to room temperature, the mixture was diluted with a 6:1 (v/v) solution of DCM/MeOH (5 mL) and passed through a pad of silica gel eluting with a 6:1 (v/v) solution of DCM/MeOH (50 mL). The volatiles were removed under reduced pressure. The crude material was purified by preparative HPLC on a C18 column (43.8-63.8% CH₃CN/0.2% TFA (aq.)) to afford the title compound as a TFA salt (21.1 mg, 54% yield), a white solid. LCMS calc. for C₂₈H₂₇N₄O₇S [M+H]⁺: m/z=563.2; Found: 563.1.

### Step 2: N-(6-((1H-Pyrazol-1-yl)methyl)-4-methoxybenzo[d]isoxazol-3-yl)-4′-((4-(3-(2,4-dioxotetrahydropyrimidin-1(2H)-yl)-1-methyl-1H-indazol-6-yl)piperidin-1-yl)methyl)-2,4-dimethoxy-3′-methyl-[1,1′-biphenyl]-3-sulfonamide

The title compound was synthesized by procedures analogous to those outlined in Example 35, Step 5, utilizing 3-(1-methyl-6-(piperidin-4-yl)-1H-indazol-3-yl)piperidine-2,6-dione (which can be prepared as described in WO2021127586 A1). LCMS calc. for C₄₅H₄₈N₉O₈S [M+H]⁺: m/z=874.3; Found: 874.3.

### Examples 63-72

Examples 63-72 listed in Table 2 were synthesized according to procedures analogous to Example 62 using appropriate starting materials. All examples in these tables were prepared as the TFA salt unless otherwise noted.

TABLE 2
Examples 63-72

| | | LCMS |
|---|---|---|
| Example | Structure/Name | [M + H]⁺ |
| 63 | 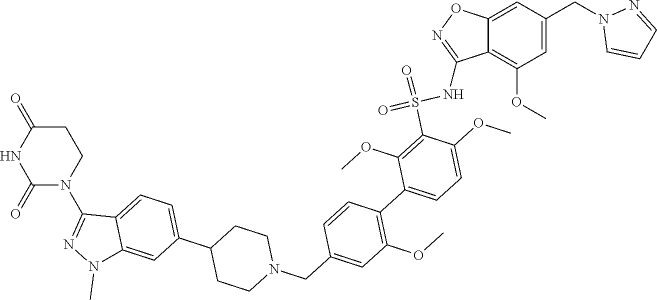 [SMILES: COc1cc(CN2CCC(c3ccc4c(N5CCC(=O)NC5=O)nn(C)c4c3)CC2)ccc1-c1ccc(OC)c(S(=O)(=O)Nc2noc3cc(Cn4cccn4)cc(OC)c23)c1OC] | 890.3 |
| | N-(6-((1H-Pyrazol-1-yl)methyl)-4-methoxybenzo[d]isoxazol-3- | |
| | yl)-4′-((4-(3-(2,4-dioxotetrahydropyrimidin-1(2H)-yl)-1-methyl- | |
| | 1H-indazol-6-yl)piperidin-1-yl)methyl)-2,2′,4-trimethoxy-[1,1′- | |
| | biphenyl]-3-sulfonamide | |
| 64 | 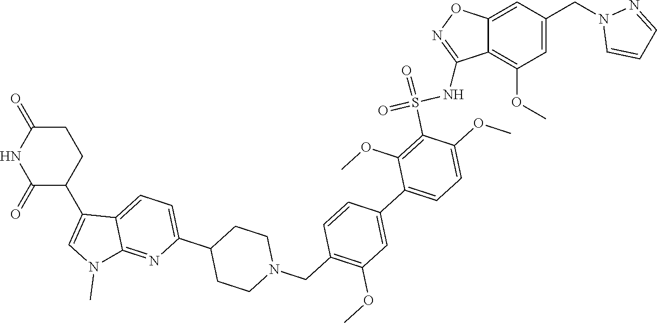 [SMILES: COc1cc(-c2ccc(OC)c(S(=O)(=O)Nc3noc4cc(Cn5cccn5)cc(OC)c34)c2OC)ccc1CN1CCC(c2ccc3c(C4CCC(=O)NC4=O)cn(C)c3n2)CC1] | 889.2 |
| | N-(6-((1H-Pyrazol-1-yl)methyl)-4-methoxybenzo[d]isoxazol-3- | |
| | yl)-4′-((4-(3-(2,6-dioxopiperidin-3-yl)-1-methyl-1H-pyrrolo[2,3- | |
| | b]pyridin-6-yl)piperidin-1-yl)methyl)-2,3′,4-trimethoxy-[1,1′- | |
| | biphenyl]-3-sulfonamide | |
| 65 | 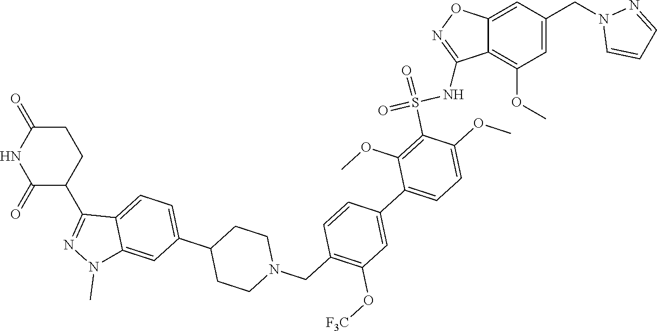 [SMILES: COc1ccc(-c2ccc(CN3CCC(c4ccc5c(C6CCC(=O)NC6=O)nn(C)c5c4)CC3)c(OC(F)(F)F)c2)c(OC)c1S(=O)(=O)Nc1noc2cc(Cn3cccn3)cc(OC)c12] | 943.3 |
| | N-(6-((1H-Pyrazol-1-yl)methyl)-4-methoxybenzo[d]isoxazol-3- | |
| | yl)-4′-((4-(3-(2,6-dioxopiperidin-3-yl)-1-methyl-1H-indazol-6- | |
| | yl)piperidin-1-yl)methyl)-2,4-dimethoxy-3′-(trifluoromethoxy)- | |
| | [1,1′-biphenyl]-3-sulfonamide | |
| 66 | 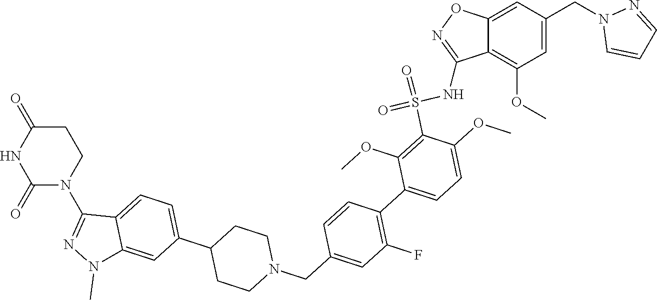 [SMILES: COc1ccc(-c2ccc(CN3CCC(c4ccc5c(N6CCC(=O)NC6=O)nn(C)c5c4)CC3)cc2F)c(OC)c1S(=O)(=O)Nc1noc2cc(Cn3cccn3)cc(OC)c12] | 878.2 |
| | N-(6-((1H-Pyrazol-1-yl)methyl)-4-methoxybenzo[d]isoxazol-3- | |
| | yl)-4′-((4-(3-(2,4-dioxotetrahydropyrimidin-1(2H)-yl)-1-methyl- | |
| | 1H-indazol-6-yl)piperidin-1-yl)methyl)-2′-fluoro-2,4-dimethoxy- | |
| | [1,1′-biphenyl]-3-sulfonamide | |
| 67 | 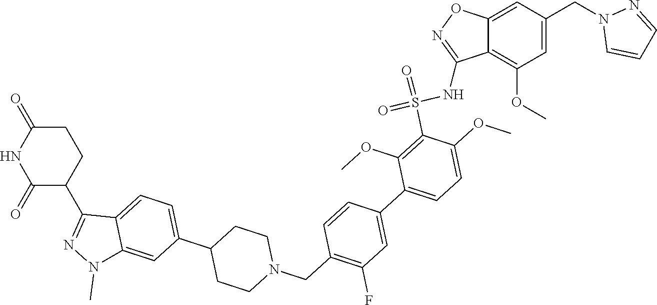 [SMILES: COc1ccc(-c2ccc(CN3CCC(c4ccc5c(C6CCC(=O)NC6=O)nn(C)c5c4)CC3)c(F)c2)c(OC)c1S(=O)(=O)Nc1noc2cc(Cn3cccn3)cc(OC)c12] | 877.1 |
| | N-(6-((1H-Pyrazol-1-yl)methyl)-4-methoxybenzo[d]isoxazol-3- | |
| | yl)-4′-((4-(3-(2,6-dioxopiperidin-3-yl)-1-methyl-1H-indazol-6- | |
| | yl)piperidin-1-yl)methyl)-3′-fluoro-2,4-dimethoxy- | |
| | [1,1′-biphenyl]-3-sulfonamide | |
| 68 | 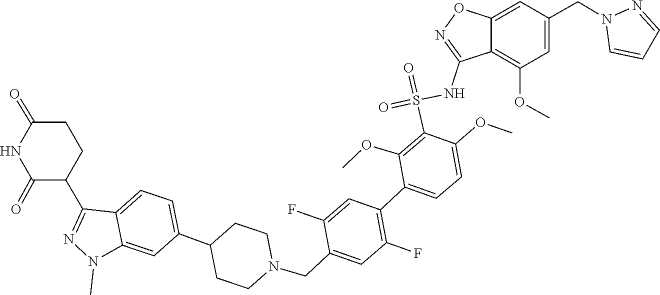 [SMILES: COc1ccc(-c2cc(F)c(CN3CCC(c4ccc5c(C6CCC(=O)NC6=O)nn(C)c5c4)CC3)cc2F)c(OC)c1S(=O)(=O)Nc1noc2cc(Cn3cccn3)cc(OC)c12] | 895.3 |
| | N-(6-((1H-Pyrazol-1-yl)methyl)-4-methoxybenzo[d]isoxazol-3- | |
| | yl)-4′-((4-(3-(2,6-dioxopiperidin-3-yl)-1-methyl-1H-indazol-6- | |
| | yl)piperidin-1-yl)methyl)-2′,5′-difluoro-2,4-dimethoxy- | |
| | [1,1′-biphenyl]-3-sulfonamide | |
| 69 | 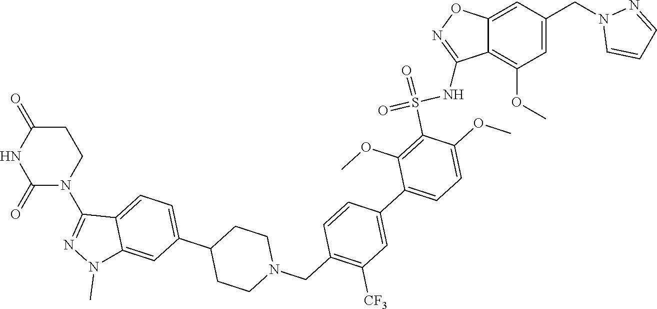 [SMILES: COc1ccc(-c2ccc(CN3CCC(c4ccc5c(N6CCC(=O)NC6=O)nn(C)c5c4)CC3)c(C(F)(F)F)c2)c(OC)c1S(=O)(=O)Nc1noc2cc(Cn3cccn3)cc(OC)c12] | 928.2 |
| | N-(6-((1H-Pyrazol-1-yl)methyl)-4-methoxybenzo[d]isoxazol-3- | |
| | yl)-4′-((4-(3-(2,4-dioxotetrahydropyrimidin-1(2H)-yl)-1-methyl- | |
| | 1H-indazol-6-yl)piperidin-1-yl)methyl)-2,4-dimethoxy-3′- | |
| | (trifluoromethyl)-[1,1′-biphenyl]-3-sulfonamide | |
| 70 | 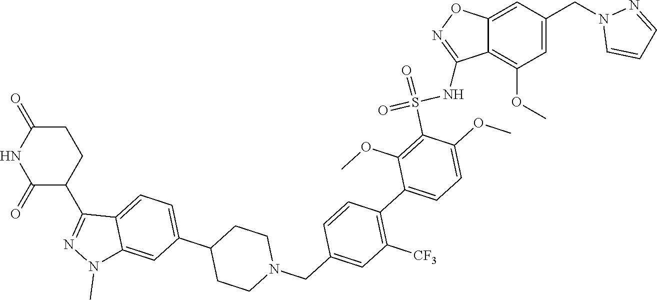 [SMILES: COc1ccc(-c2ccc(CN3CCC(c4ccc5c(C6CCC(=O)NC6=O)nn(C)c5c4)CC3)cc2C(F)(F)F)c(OC)c1S(=O)(=O)Nc1noc2cc(Cn3cccn3)cc(OC)c12] | 927.3 |
| | N-(6-((1H-Pyrazol-1-yl)methyl)-4-methoxybenzo[d]isoxazol-3- | |
| | yl)-4′-((4-(3-(2,6-dioxopiperidin-3-yl)-1-methyl-1H-indazol-6- | |
| | yl)piperidin-1-yl)methyl)-2,4-dimethoxy-2′-(trifluoromethyl)- | |
| | [1,1′-biphenyl]-3-sulfonamide | |
| 71 | 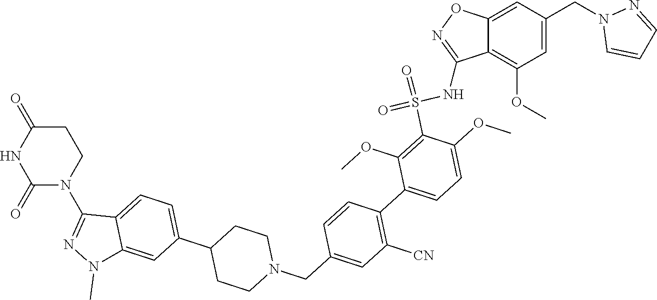 [SMILES: COc1ccc(-c2ccc(CN3CCC(c4ccc5c(N6CCC(=O)NC6=O)nn(C)c5c4)CC3)cc2C#N)c(OC)c1S(=O)(=O)Nc1noc2cc(Cn3cccn3)cc(OC)c12] | 885.3 |
| | N-(6-((1H-Pyrazol-1-yl)methyl)-4-methoxybenzo[d]isoxazol-3- | |
| | yl)-2′-cyano-4′-((4-(3-(2,4-dioxotetrahydropyrimidin-1(2H)-yl)- | |
| | 1-methyl-1H-indazol-6-yl)piperidin-1-yl)methyl)-2,4- | |
| | dimethoxy-[1,1′-biphenyl]-3-sulfonamide | |
| 72 | 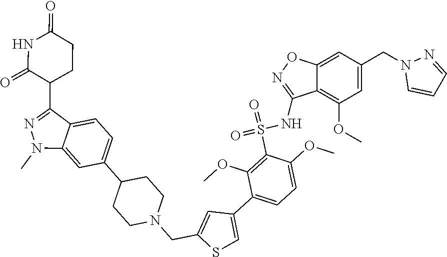 [SMILES: COc1ccc(-c2csc(CN3CCC(c4ccc5c(C6CCC(=O)NC6=O)nn(C)c5c4)CC3)c2)c(OC)c1S(=O)(=O)Nc1noc2cc(Cn3cccn3)cc(OC)c12] | 865.2 |
| | N-(6-((1H-Pyrazol-1-yl)methyl)-4-methoxybenzo[d]isoxazol-3- | |
| | yl)-3-(5-((4-(3-(2,6-dioxopiperidin-3-yl)-1-methyl-1H-indazol-6- | |
| | yl)piperidin-1-yl)methyl)thiophen-3-yl)-2,6- | |
| | dimethoxybenzenesulfonamide | |

### Example 73. N-(6-((1H-Pyrazol-1-yl)methyl)-4-methoxybenzo[d]isoxazol-3-yl)-3-(2-((1-(3-(2,6-dioxopiperidin-3-yl)-1-methyl-1H-indazol-6-yl)piperidin-4-yl)methyl)-1,2,3,4-tetrahydroisoquinolin-6-yl)-5-fluoro-2,6-dimethoxybenzenesulfonamide

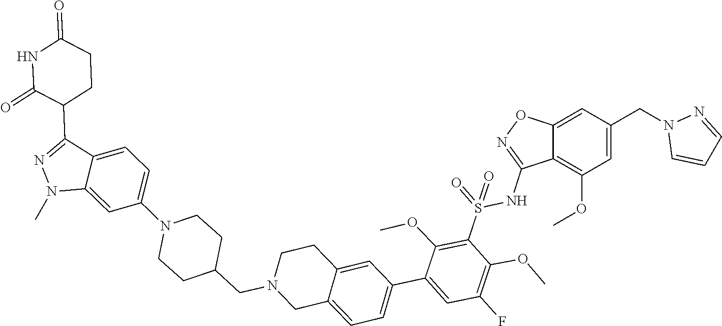 [SMILES: COc1c(F)cc(-c2ccc3c(c2)CCN(CC2CCN(c4ccc5c(C6CCC(=O)NC6=O)nn(C)c5c4)CC2)C3)c(OC)c1S(=O)(=O)Nc1noc2cc(Cn3cccn3)cc(OC)c12]

### Step 1: N-(6-((1H-Pyrazol-1-yl)methyl)-4-methoxybenzo[d]isoxazol-3-yl)-3-fluoro-2,6-dimethoxy-5-(1, 2,3,4-tetrahydroisoquinolin-6-yl)benzenesulfonamide

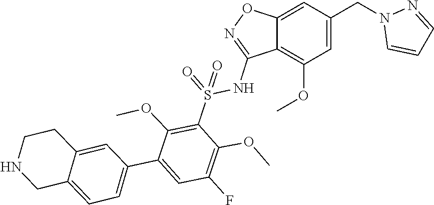 [SMILES: COc1c(F)cc(-c2ccc3c(c2)CCNC3)c(OC)c1S(=O)(=O)Nc1noc2cc(Cn3cccn3)cc(OC)c12]

To a solution of N-(6-((1H-pyrazol-1-yl)methyl)-4-methoxybenzo[d]isoxazol-3-yl)-3-bromo-5-fluoro-2,6-dimethoxybenzenesulfonamide (20.0 mg, 0.040 mmol, Example 55, Step 3) in water (0.20 mL) and 1,4-dioxane (1 mL) was sparged with nitrogen for 3 min. Potassium carbonate (22 mg, 0.16 mmol), XPhos Pd G4 (CAS: 1599466-81-5) (7 mg, 0.008 mmol), and (2-(tert-butoxycarbonyl)-1,2,3,4-tetrahydroisoquinolin-6-yl)boronic acid (20 mg, 0.080 mmol) were added. The mixture was stirred at 80° C. for 40 minutes, cooled to room temperature, diluted with a 6:1 (v/v) solution of DCM/MeOH (5 mL), and passed through a pad of silica gel eluting with a 6:1 (v/v) solution of DCM/MeOH (50 mL). The volatiles were removed under reduced pressure. The resulting material was stirred with DCM (1 mL) and 2,2,2-trifluoroacetic acid (0.31 mL, 4.0 mmol) at room temperature for 30 minutes. The mixture was diluted with MeCN (3 mL) and partially concentrated. The crude material was purified by preparative HPLC on a C18 column (24.5-44.5% CH₃CN/0.2% TFA (aq.)) to afford the title compound as TFA salt (12.8 mg, 45% yield) as white solid. LC-MS calc. for C₂₉H₂₉FN₅O₆S [M+H]⁺: m/z=594.2; Found: 594.2.

### Step 2: N-(6-((1H-Pyrazol-1-yl)methyl)-4-methoxybenzo[d]isoxazol-3-yl)-3-(2-((1-(3-(2,6-dioxopiperidin-3-yl)-1-methyl-1H-indazol-6-yl)piperidin-4-yl)methyl)-1,2,3,4-tetrahydroisoquinolin-6-yl)-5-fluoro-2,6-dimethoxybenzenesulfonamide

The title compound was synthesized by procedures analogous to those outlined in Example 2, using 1-(3-(2,4-dioxotetrahydropyrimidin-1(2H)-yl)-1-methyl-1H-indazol-6-yl)piperidine-4-carbaldehyde (which can be prepared as described in WO2023249970A1). ¹H NMR (400 MHz, CD₃OD) δ 7.81 (d, J=2.3 Hz, 1H), 7.64 (d, J=8.9 Hz, 1H), 7.59 (d, J=1.9 Hz, 1H), 7.47 (d, J=11.8 Hz, 1H), 7.42 (m, 2H), 7.33 (d, J=8.3 Hz, 1H), 7.07 (dd, J=8.9, 1.9 Hz, 1H), 7.03 (d, J=1.9 Hz, 1H), 6.83 (s, 1H), 6.72 (s, 1H), 6.40 (t, J=2.1 Hz, 1H), 5.51 (s, 2H), 4.73 (br s, 1H), 4.48 (br s, 1H), 4.34 (dd, J=9.2, 5.1 Hz, 1H), 4.04 (d, J=1.8 Hz, 3H), 3.99 (s, 3H), 3.98 (s, 3H), 3.91 (d, J=12.8 Hz, 3H), 3.61-3.47 (m, 1H), 3.43 (s, 3H), 3.38-3.33 (m, 2H), 3.31-3.17 (br m, 2H), 3.05 (t, J=12.1 Hz, 2H), 2.86-2.69 (m, 2H), 2.46 (ddt, J=14.3, 9.0, 4.4 Hz, 1H), 2.37-2.25 (m, 2H), 2.06 (d, J=12.8 Hz, 2H), 1.65 (q, J=12.0 Hz, 2H). ¹⁹F NMR (376 MHz, CD₃OD) δ −77.33, −133.03. LC-MS calc. for C₄₈H₅₁FN₉O₈S [M+H]⁺: m/z=932.4; Found: 932.2.

### Examples 74-76 and 119

Examples 74-76 and 119 listed in Table 3 were synthesized according to procedures analogous to Example 73 using appropriate starting materials. All examples in these tables were prepared as the TFA salt unless otherwise noted.

TABLE 3
Examples 74-76 and 119

| | | LCMS |
|---|---|---|
| Example | Structure/Name | [M + H]⁺ |
| 74 | 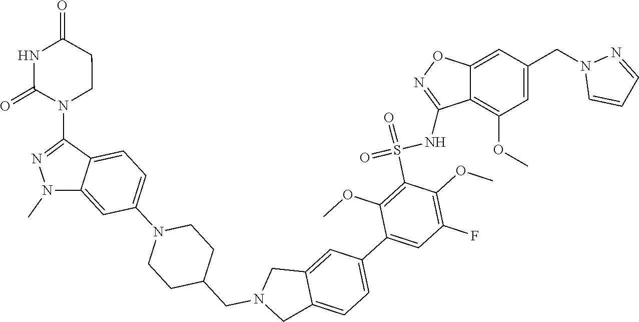 [SMILES: COc1c(F)cc(-c2ccc3c(c2)CN(CC2CCN(c4ccc5c(N6CCC(=O)NC6=O)nn(C)c5c4)CC2)C3)c(OC)c1S(=O)(=O)Nc1noc2cc(Cn3cccn3)cc(OC)c12] | 919.3 |
| | N-(6-((1H-Pyrazol-1-yl)methyl)-4-methoxybenzo[d]isoxazol-3-yl)-3- | |
| | (2-((1-(3-(2,4-dioxotetrahydropyrimidin-1(2H)-yl)-1-methyl-1H- | |
| | indazol-6-yl)piperidin-4-yl)methyl)isoindolin-5-yl)-5-fluoro-2,6- | |
| | dimethoxybenzenesulfonamide | |
| 75 | 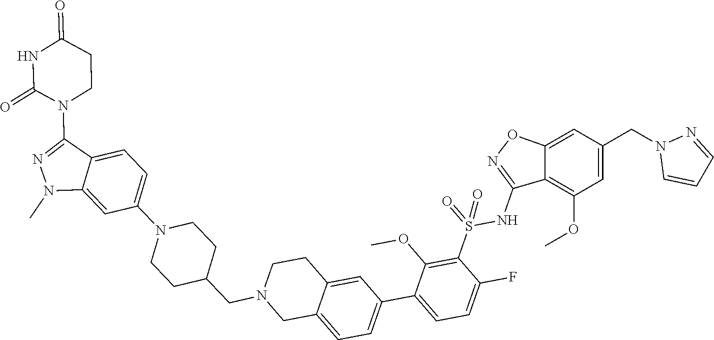 [SMILES: COc1c(-c2ccc3c(c2)CCN(CC2CCN(c4ccc5c(N6CCC(=O)NC6=O)nn(C)c5c4)CC2)C3)ccc(F)c1S(=O)(=O)Nc1noc2cc(Cn3cccn3)cc(OC)c12] | 903.3 |
| | N-(6-((1H-Pyrazol-1-yl)methyl)-4-methoxybenzo[d]isoxazol-3-yl)-3- | |
| | (2-((1-(3-(2,4-dioxotetrahydropyrimidin-1(2H)-yl)-1-methyl-1H- | |
| | indazol-6-yl)piperidin-4-yl)methyl)-1,2,3,4-tetrahydroisoquinolin-6- | |
| | yl)-6-fluoro-2-methoxybenzenesulfonamide | |
| 76 | 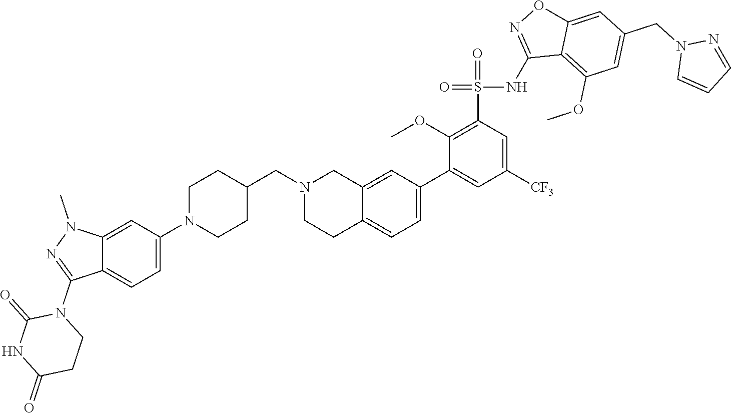 [SMILES: COc1c(-c2ccc3c(c2)CN(CC2CCN(c4ccc5c(N6CCC(=O)NC6=O)nn(C)c5c4)CC2)CC3)cc(C(F)(F)F)cc1S(=O)(=O)Nc1noc2cc(Cn3cccn3)cc(OC)c12] | 953.3 |
| | N-(6-((1H-Pyrazol-1-yl)methyl)-4-methoxybenzo[d]isoxazol-3-yl)-3- | |
| | (2-((1-(3-(2,4-dioxotetrahydropyrimidin-1(2H)-yl)-1-methyl-1H- | |
| | indazol-6-yl)piperidin-4-yl)methyl)-1,2,3,4-tetrahydroisoquinolin-7- | |
| | yl)-2-methoxy-5-(trifluoromethyl)benzenesulfonamide | |
| 119 | 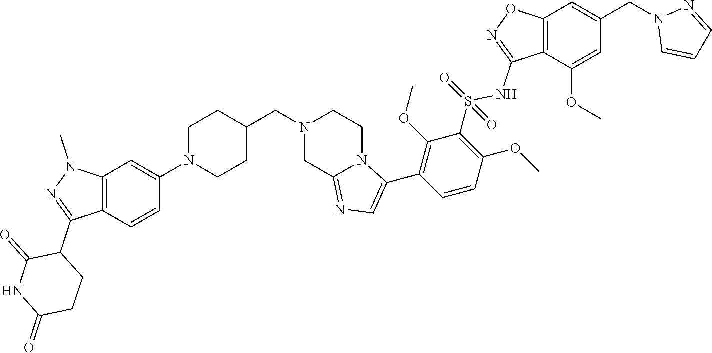 [SMILES: COc1ccc(-c2cnc3n2CCN(CC2CCN(c4ccc5c(C6CCC(=O)NC6=O)nn(C)c5c4)CC2)C3)c(OC)c1S(=O)(=O)Nc1noc2cc(Cn3cccn3)cc(OC)c12] | 904.2 |
| | N-(6-((1H-Pyrazol-1-yl)methyl)-4-methoxybenzo[d]isoxazol-3-yl)-3- | |
| | (7-((1-(3-(2,6-dioxopiperidin-3-yl)-1-methyl-1H-indazol-6- | |
| | yl)piperidin-4-yl)methyl)-5,6,7,8-tetrahydroimidazo[1,2-a]pyrazin-3- | |
| | yl)-2,6-dimethoxybenzenesulfonamide | |

### Example 77. N-(6-((1H-Pyrazol-1-yl)methyl)-4-methoxybenzo[d]isoxazol-3-yl)-5-(tert-butyl)-3-(6-((1-(3-(2,4-dioxotetrahydropyrimidin-1(2H)-yl)-1-methyl-1H-indazol-6-yl)piperidin-4-yl)methyl)-5,6,7,8-tetrahydro-1,6-naphthyridin-2-yl)-2-methoxybenzene-sulfonamide

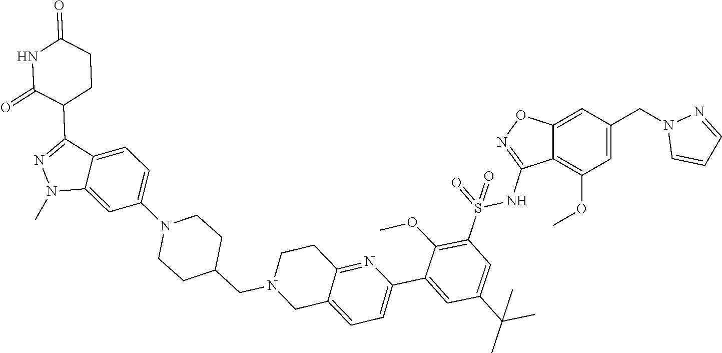 [SMILES: COc1c(-c2ccc3c(n2)CCN(CC2CCN(c4ccc5c(C6CCC(=O)NC6=O)nn(C)c5c4)CC2)C3)cc(C(C)(C)C)cc1S(=O)(=O)Nc1noc2cc(Cn3cccn3)cc(OC)c12]

### Step 1: 1,3-Dibromo-5-(tert-butyl)-2-methoxybenzene

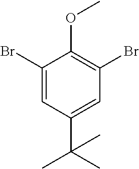

A solution of potassium carbonate (2870 mg, 20.8 mmol), 2,6-dibromo-4-tert-butyl-phenol (3200 mg, 10.4 mmol), and iodomethane (2.59 mL, 41.6 mmol) in acetone (25 mL) was stirred at 60° C. in a sealed vial for 4 h. After cooling to room temperature, the mixture was passed through a pad of silica gel, eluting with ethyl acetate (100 mL) and the volatiles were removed under reduced pressure. The crude material was purified by silica gel chromatography (0-50% DCM/hexanes) to afford the title compound (3310 mg, 98.9% yield) as colorless oil. ¹H NMR (400 MHz, CDCl₃) δ 7.50 (s, 2H), 3.89 (s, 3H), 1.31 (s, 9H).

### Step 2: Benzyl(3-bromo-5-(tert-butyl)-2-methoxyphenyl)sulfane

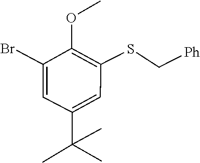 [SMILES: COc1c(Br)cc(C(C)(C)C)cc1SCc1ccccc1]

To a solution of tris(dibenzylideneacetone)dipalladium(0) (0.23 g, 0.25 mmol), phenylmethanethiol (1.24 mL, 10.6 mmol), N,N-diisopropylethylamine (3.50 mL, 20.1 mmol), (9,9-dimethyl-9H-xanthene-4,5-diyl)bis(diphenylphosphane) (291 mg, 0.502 mmol), 1,3-dibromo-5-(tert-butyl)-2-methoxybenzene (3.24 g, 10.0 mmol) in 1,4-dioxane (25 mL) was sparged with nitrogen for 5 minutes. The mixture was stirred at 100° C. under a nitrogen atmosphere for 3 h. After cooling to room temperature, the mixture was passed through a pad of silica gel, eluting with ethyl acetate (80 mL). The volatiles were removed under reduced pressure and the crude material was purified by silica gel chromatography (0-20% DCM/hexanes) to afford the title compound (1.6 g, 42% yield) as white solid. ¹H NMR (400 MHz, CDCl₃) δ 7.37 (d, J=2.3 Hz, 1H), 7.34-7.20 (m, 5H), 7.12 (d, J=2.3 Hz, 1H), 4.14 (s, 2H), 3.90 (s, 3H), 1.22 (s, 9H).

### Step 3: 3-Bromo-5-(tert-butyl)-2-methoxybenzenesulfonyl chloride

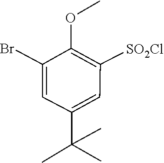 [SMILES: COc1c(Br)cc(C(C)(C)C)cc1S(=O)(=O)Cl]

A solution of benzyl(3-bromo-5-(tert-butyl)-2-methoxyphenyl)sulfane (1.55 g, 4.24 mmol) in acetic acid (6 mL) and water (4 mL) was stirred for 5 min. 1-Chloropyrrolidine-2,5-dione (2.27 g, 17.0 mmol) was added and the mixture was stirred for 5 h. The reaction was quenched by brine (10 mL) and extracted with DCM (3×30 mL). The combined organic phases were dried over anhydrous MgSO₄, filtered, and concentrated. The crude material was purified by silica gel chromatography (0-20% DCM/hexanes) to afford the title compound (1.25 g, 86.0% yield) as colorless oil. ¹H NMR (400 MHz, CDCl₃) δ 7.92 (s, 1H), 7.91 (s, 1H), 4.12 (s, 3H), 1.37 (s, 9H).

### Step 4: N-(6-((1H-Pyrazol-1-yl)methyl)-4-methoxybenzo[d]isoxazol-3-yl)-3-bromo-5-(tert-butyl)-2-methoxybenzenesulfonamide

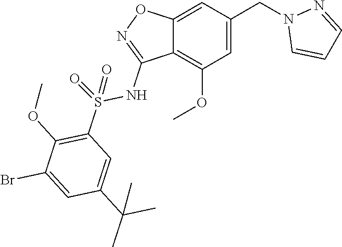 [SMILES: COc1c(Br)cc(C(C)(C)C)cc1S(=O)(=O)Nc1noc2cc(Cn3cccn3)cc(OC)c12]

The title compound was synthesized by procedures analogous to those outlined in Example 12, Step 1. LCMS calc. for C₂₃H₂₆BrN₄O₅S [M+H]⁺: m/z=549.1, 551.1; Found: 549.0, 551.0.

### Step 5: N-(6-((1H-Pyrazol-1-yl)methyl)-4-methoxybenzo[d]isoxazol-3-yl)-5-(tert-butyl)-2-methoxy-3-(4,4,5,5-tetramethyl-1, 3,2-dioxaborolan-2-yl)benzenesulfonamide

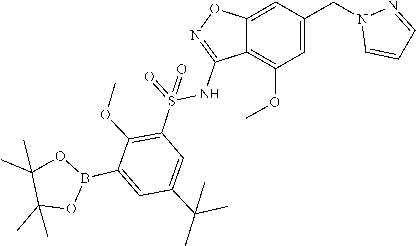 [SMILES: COc1c(B2OC(C)(C)C(C)(C)O2)cc(C(C)(C)C)cc1S(=O)(=O)Nc1noc2cc(Cn3cccn3)cc(OC)c12]

A solution of N-(6-((1H-pyrazol-1-yl)methyl)-4-methoxybenzo[d]isoxazol-3-yl)-3-bromo-5-(tert-butyl)-2-methoxybenzenesulfonamide (100 mg, 0.182 mmol) in 1,4-dioxane (2 mL) was sparged with nitrogen for 5 min. Potassium 2,2-dimethylpropanoate (68.9 mg, 0.491 mmol), bis(pinacolato)diboron (69.3 mg, 0.273 mmol), and [1,1′-bis(diphenylphosphino)-ferrocene]dichloropalladium(II), complex with dichloromethane (30 mg, 0.036 mmol) were added. The mixture was stirred at 90° C. under a nitrogen atmosphere for 1 h. After cooling to room temperature, the mixture was diluted with ethyl acetate (20 mL), passed through a pad of silica gel (10 g) eluting with a 1:6 (v/v) solution of MeOH/DCM (70 mL), and concentrated. The crude material was used in the next step without further purification. LCMS calc. for C₂₉H₃₈BN₄O₇S [M+H]⁺: m/z=597.3; Found: 597.2.

### Step 6: N-(6-((1H-Pyrazol-1-yl)methyl)-4-methoxybenzo[d]isoxazol-3-yl)-5-(tert-butyl)-2-methoxy-3-(5,6,7,8-tetrahydro-1, 6-naphthyridin-2-yl)benzenesulfonamide

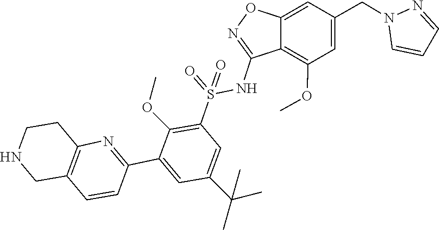 [SMILES: COc1c(-c2ccc3c(n2)CCNC3)cc(C(C)(C)C)cc1S(=O)(=O)Nc1noc2cc(Cn3cccn3)cc(OC)c12]

The title compound was synthesized by procedures analogous to those outlined in Example 61, Step 3. LCMS calc. for C₃₁H₃₅N₆O₅S [M+H]⁺: m/z=603.2; Found: 603.1.

### Step 7: N-(6-((1H-pyrazol-1-yl)methyl)-4-methoxybenzo[d]isoxazol-3-yl)-5-(tert-butyl)-3-(6-((1-(3-(2,4-dioxotetrahydropyrimidin-1(2H)-yl)-1-methyl-1H-indazol-6-yl)piperidin-4-yl)methyl)-5,6,7,8-tetrahydro-1, 6-naphthyridin-2-yl)-2-methoxybenzene-sulfonamide

The title compound was synthesized by procedures analogous to those outlined in Example 2. ¹H NMR (400 MHz, CD₃OD) δ 8.12 (d, J=2.6 Hz, 1H), 7.93 (d, J=2.6 Hz, 1H), 7.80 (d, J=2.3 Hz, 1H), 7.75 (d, J=8.1 Hz, 1H), 7.64-7.57 (m, 2H), 7.55 (d, J=8.1 Hz, 1H), 7.06 (dd, J=9.1, 1.9 Hz, 1H), 6.99 (s, 1H), 6.83 (d, J=1.0 Hz, 1H), 6.69 (d, J=1.0 Hz, 1H), 6.40 (t, J=2.1 Hz, 1H), 5.49 (s, 2H), 4.67 (br s, 2H), 4.04 (t, J=6.7 Hz, 2H), 3.98 (s, 3H), 3.96-3.73 (m, 7H), 3.50 (s, 3H), 3.43 (br s, 2H), 3.38 (d, J=7.0 Hz, 2H), 3.36-3.33 (m, 1H), 3.04 (t, J=12.1 Hz, 2H), 2.88 (t, J=6.7 Hz, 2H), 2.38-2.24 (m, 1H), 2.07 (br d, J=12.9 Hz, 2H), 1.65 (q, J=12.7, 12.3 Hz, 1H), 1.39 (s, 9H). ¹⁹F NMR (376 MHz, CD₃OD) δ −77.3. LCMS calc. for C₄₉H₅₆N₁₁O₇S [M+H]⁺: m/z=942.4; Found: 942.0.

### Example 78. N-(6-((1H-Pyrazol-1-yl)methyl)-4-methoxybenzo[d]isoxazol-3-yl)-3-(2-((4-(3-(2,6-dioxopiperidin-3-yl)-1-methyl-1H-indazol-6-yl)piperidin-1-yl)methyl)pyrimidin-5-yl)-2,6-dimethoxybenzenesulfonamide

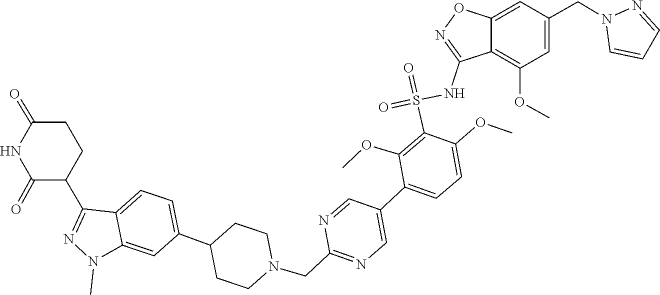 [SMILES: COc1ccc(-c2cnc(CN3CCC(c4ccc5c(C6CCC(=O)NC6=O)nn(C)c5c4)CC3)nc2)c(OC)c1S(=O)(=O)Nc1noc2cc(Cn3cccn3)cc(OC)c12]

### Step 1: 5-Bromo-2-vinylpyrimidine

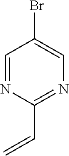

A solution of 2,5-dibromopyrimidine (1.0 g, 4.2 mmol) in water (4 mL) and 1,4-dioxane (18 mL) was sparged for 5 min. [1,1′-Bis(diphenylphosphino)ferrocene]dichloro-palladium(II), complex with dichloromethane (0.34 g, 0.42 mmol), 4,4,5,5-tetramethyl-2-vinyl-1,3,2-dioxaborolane (0.68 g, 4.4 mmol), and potassium carbonate (1.16 g, 8.41 mmol) were added. The mixture was stirred at 90° C. for 3 h. After cooling to room temperature, the reaction was diluted with ethyl acetate (20 mL) and brine (10 mL). The phases were separated, and the aqueous phase was extracted with ethyl acetate (30 mL). The combined organic phases were dried over anhydrous MgSO₄, filtered, and concentrated. The crude material was purified by silica gel chromatography (0-100% DCM/hexanes) to afford the title compound (462 mg, 59.4% yield) as white solid. LCMS calc. for C₆H₆BrN₂ [M+H]⁺: m/z=185.0, 187.0; Found: 184.9, 186.9.

### Step 2: 5-(4,4,5,5-Tetramethyl-1, 3,2-dioxaborolan-2-yl)-2-vinylpyrimidine

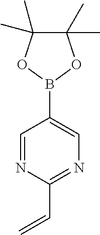

The title compound was synthesized by procedures analogous to those outlined in Example 77, Step 5. LCMS calc. for C₁₂H₁₈BN₂O₂ [M+H]⁺: m/z=233.1; Found: 233.1.

### Step 3: N-(6-((1H-Pyrazol-1-yl)methyl)-4-methoxybenzo[d]isoxazol-3-yl)-2,6-dimethoxy-3-(2-vinylpyrimidin-5-yl)benzenesulfonamide

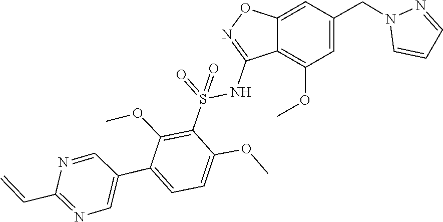 [SMILES: C=Cc1ncc(-c2ccc(OC)c(S(=O)(=O)Nc3noc4cc(Cn5cccn5)cc(OC)c34)c2OC)cn1]

A solution of N-(6-((1H-pyrazol-1-yl)methyl)-4-methoxybenzo[d]isoxazol-3-yl)-3-bromo-2,6-dimethoxybenzenesulfonamide (40 mg, 0.076 mmol) (which can be prepared as described in WO2022235945 A1) in water (0.30 mL) and 1,4-dioxane (2.0 mL) was sparged with nitrogen for 3 min. Potassium carbonate (63 mg, 0.46 mmol), XPhos Pd G4 (CAS: 1599466-81-5, 13.2 mg, 0.0153 mmol), and 5-(4,4,5,5-tetramethyl-1,3,2-dioxaborolan-2-yl)-2-vinylpyrimidine (88.7 mg, 0.382 mmol) were added. The mixture was stirred at 90° C. under a nitrogen atmosphere for 40 min. After cooling to room temperature, the mixture was diluted with a 6:1 (v/v) solution of DCM/MeOH (5 mL) and passed through a pad of silica gel eluting with a 6:1 (v/v) solution of DCM/MeOH (50 mL). The volatiles were removed under reduced pressure. The material was carried forward without further purification. LCMS calc. for C₂₆H₂₅N₆O₆S [M+H]⁺: m/z=549.2; Found: 549.0.

### Step 4: N-(6-((1H-Pyrazol-1-yl)methyl)-4-methoxybenzo[d]isoxazol-3-yl)-3-(2-formylpyrimidin-5-yl)-2,6-dimethoxybenzenesulfonamide

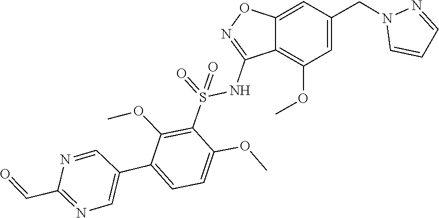 [SMILES: COc1ccc(-c2cnc(C=O)nc2)c(OC)c1S(=O)(=O)Nc1noc2cc(Cn3cccn3)cc(OC)c12]

To a solution of N-(6-((1H-pyrazol-1-yl)methyl)-4-methoxybenzo[d]isoxazol-3-yl)-2,6-dimethoxy-3-(2-vinylpyrimidin-5-yl)benzenesulfonamide (31 mg, 0.056 mmol) and 2,6-lutidine (13.1 μL, 0.113 mmol) in 1,4-dioxane (1.5 mL) and water (0.50 mL) was added osmium tetroxide (35.9 μL, 5.65 μmol, 4 wt % in water) and sodium periodate (36 mg, 0.17 mmol). The mixture was stirred for 3 h, then diluted with a 6:1 (v/v) solution of DCM/MeOH (10 mL), dried with anhydrous MgSO₄, filtered through a pad of silica gel (10 g) eluting with a 6:1 (v/v) solution of DCM/MeOH (30 mL), and concentrated under reduced pressure to afford the title compound as brown semi-solid which was used without further purification. LCMS calc. for C₂₅H₂₃N₆O₇S [M+H]⁺: m/z=551.1; Found: 551.0.

### Step 5: N-(6-((1H-Pyrazol-1-yl)methyl)-4-methoxybenzo[d]isoxazol-3-yl)-3-(2-((4-(3-(2,6-dioxopiperidin-3-yl)-1-methyl-1H-indazol-6-yl)piperidin-1-yl)methyl)pyrimidin-5-yl)-2,6-dimethoxybenzenesulfonamide

The title compound was synthesized by procedures analogous to those outlined in Example 62, Step 2. LCMS calc. for C₄₃H₄₅N₁₀O₈S [M+H]⁺: m/z=861.3; Found: 861.2.

### Example 79. N-(6-((1H-Pyrazol-1-yl)methyl)-4-methoxybenzo[d]isoxazol-3-yl)-3-(6-((4-(3-(2,6-dioxopiperidin-3-yl)-1-methyl-1H-indazol-6-yl)piperidin-1-yl)methyl)-2-methylpyridin-3-yl)-5-fluoro-2,6-dimethoxybenzenesulfonamide

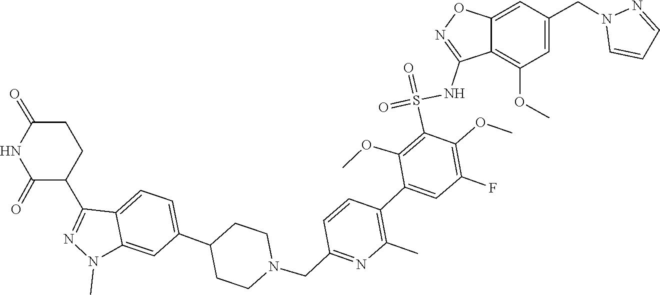 [SMILES: COc1c(F)cc(-c2ccc(CN3CCC(c4ccc5c(C6CCC(=O)NC6=O)nn(C)c5c4)CC3)nc2C)c(OC)c1S(=O)(=O)Nc1noc2cc(Cn3cccn3)cc(OC)c12]

### Step 1: 3-Bromo-2-methyl-6-vinylpyridine

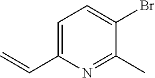

The title compound was synthesized by procedures analogous to those outlined in Example 78, Step 1. LCMS calc. for C₈H₉BrN [M+H]⁺: m/z=198.0, 200.0; Found: 198.0, 200.0.

### Step 2: (2-Methyl-6-vinylpyridin-3-yl)boronic acid

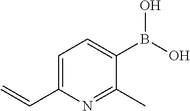

A solution of 3-bromo-2-methyl-6-vinylpyridine (73 mg, 0.37 mmol) in THF (1 mL) was stirred at −78° C. for 5 min then n-Butyllithium (0.18 mL, 0.46 mmol, 2.5 M in hexanes) was added dropwise. Stirring continued for 15 min at −78° C., after which trimethyl borate (50 μL, 0.46 mmol) was added drop wise. The mixture was stirred for 20 minutes at −78° C., then was allowed to warm to room temperature over 1 h. The reaction was quenched with saturated NH₄Cl (aq.) (10 mL) and extracted with a 6:1 (v/v) solution of DCM/MeOH (3×20 mL). The combined organic phases were dried over anhydrous MgSO₄, filtered, and concentrated to afford the title compound (49 mg, 82% yield) as white solid. LCMS calc. for C₈H₁₁BNO₂ [M+H]⁺: m/z=164.1; Found: 164.0.

### Step 3: N-(6-((1H-Pyrazol-1-yl)methyl)-4-methoxybenzo[d]isoxazol-3-yl)-3-(6-((4-(3-(2,6-dioxopiperidin-3-yl)-1-methyl-1H-indazol-6-yl)piperidin-1-yl)methyl)-2-methylpyridin-3-yl)-5-fluoro-2,6-dimethoxybenzenesulfonamide

The title compound was synthesized by procedures analogous to those outlined in Example 78, Steps 3-5. LCMS calc. for C₄₅H₄₇FN₉O₈S [M+H]⁺: m/z=892.3; Found: 892.2.

### Example 80. N-(6-((1H-Pyrazol-1-yl)methyl)-4-methoxybenzo[d]isoxazol-3-yl)-4′-(4-(3-(2,6-dioxopiperidin-3-yl)-1-methyl-1H-indazol-6-yl)piperidine-1-carbonyl)-2,4-dimethoxy-[1,1′-biphenyl]-3-sulfonamide

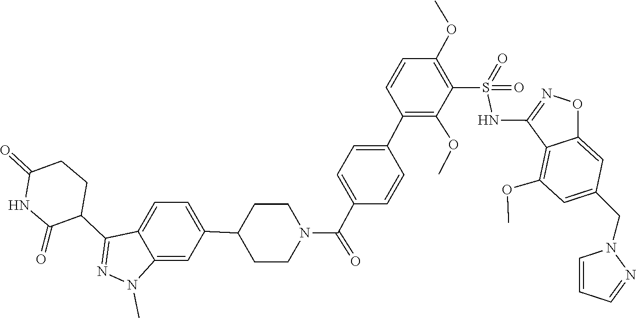 [SMILES: COc1ccc(-c2ccc(C(=O)N3CCC(c4ccc5c(C6CCC(=O)NC6=O)nn(C)c5c4)CC3)cc2)c(OC)c1S(=O)(=O)Nc1noc2cc(Cn3cccn3)cc(OC)c12]

### Step 1: 3′—(N-(6-((1H-Pyrazol-1-yl)methyl)-4-methoxybenzo[d]isoxazol-3-yl)sulfamoyl)-2′,4′-dimethoxy-[1,1′-biphenyl]-4-carboxylic acid

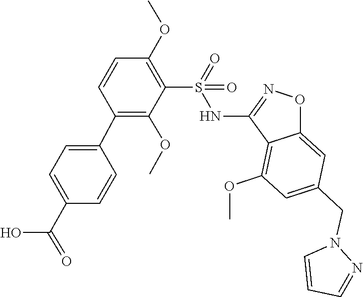 [SMILES: COc1ccc(-c2ccc(C(=O)O)cc2)c(OC)c1S(=O)(=O)Nc1noc2cc(Cn3cccn3)cc(OC)c12]

The title compound was synthesized by procedures analogous to those outlined in Example 78, Step 3, utilizing 4-boronobenzoic acid. LC-MS calc. for C₂₇H₂₅N₄O₈S [M+H]⁺: m/z=565.1; Found: 565.1.

### Step 2: N-(6-((1H-Pyrazol-1-yl)methyl)-4-methoxybenzo[d]isoxazol-3-yl)-4′-(4-(3-(2,6-dioxopiperidin-3-yl)-1-methyl-1H-indazol-6-yl)piperidine-1-carbonyl)-2,4-dimethoxy-[1,1′-biphenyl]-3-sulfonamide

A solution of 3′-(N-(6-((1H-pyrazol-1-yl)methyl)-4-methoxybenzo[d]isoxazol-3-yl)-sulfamoyl)-2′,4′-dimethoxy-[1,1′-biphenyl]-4-carboxylic acid (5.0 mg, 0.0089 mmol), 1-hydroxybenzotriazole hydrate (3.6 mg, 0.027 mmol) and [dimethylamino-(3-oxido-triazolo[4,5-b]pyridin-3-ium-1-yl)methylidene]-dimethylazanium hexafluorophosphate (10 mg, 0.027 mmol) in DMF (0.50 mL) was stirred for 15 min. 3-(1-methyl-6-(piperidin-4-yl)-1H-indazol-3-yl)piperidine-2,6-dione hydrochloride (4.8 mg, 0.013 mmol) and N,N-diisopropylethylamine (9.3 μL, 0.053 mmol) were added and stirring continued for 20 min. The crude material was then purified by preparative HPLC on a C18 column (36.8-56.8% CH₃CN/0.2% TFA (aq.)) to afford the title compound (5.1 mg, 58% yield) as a TFA salt, a white solid. LCMS calc. for C₄₅H₄₅N₈O₉S [M+H]⁺: m/z=873.3; Found: 873.2.

### Example 81. N-(6-((1H-Pyrazol-1-yl)methyl)-4-methoxybenzo[d]isoxazol-3-yl)-3-(1-((1-(3-(2,4-dioxotetrahydropyrimidin-1(2H)-yl)-1-methyl-1H-indazol-6-yl)piperidin-4-yl)methyl)piperidin-4-yl)-5-fluoro-2,6-dimethoxybenzenesulfonamide

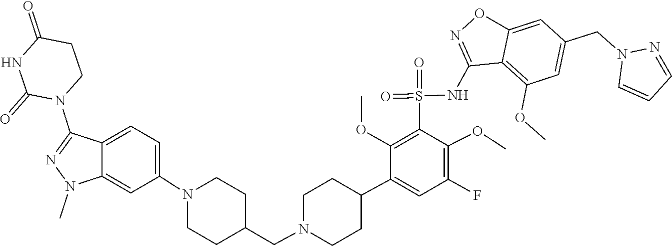 [SMILES: COc1c(F)cc(C2CCN(CC3CCN(c4ccc5c(N6CCC(=O)NC6=O)nn(C)c5c4)CC3)CC2)c(OC)c1S(=O)(=O)Nc1noc2cc(Cn3cccn3)cc(OC)c12]

### Step 1. (1-(tert-Butoxycarbonyl)piperidin-4-yl)zinc(II) iodide

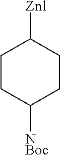

To a dried mixture of zinc (315 mg, 4.82 mmol) and lithium chloride (204 mg, 4.82 mmol) under a nitrogen atmosphere was added THF (1.6 mL), chlorotrimethylsilane (41 μL, 0.32 mmol) and 1,2-dibromoethane (28 μL, 0.32 mmol). The mixture was heated to 45° C. until bubbling began, then stirred at the same temperature for an additional 10 min. The reaction was cooled to room temperature, and a solution of tert-butyl 4-iodopiperidine-1-carboxylate (1.0 g, 3.2 mmol) in THF (1.6 mL) was added under a nitrogen atmosphere. The mixture was stirred at 45° C. overnight. The mixture was filtered through a 0.2 μm PTFE syringe filter to afford a clear, 0.6 M solution of the title compound in THF, which was stored under nitrogen.

### Step 2. tert-Butyl 4-(3-(N-(6-((1H-pyrazol-1-yl)methyl)-4-methoxybenzo[d]isoxazol-3-yl)sulfamoyl)-5-fluoro-2,4-dimethoxyphenyl)piperidine-1-carboxylate

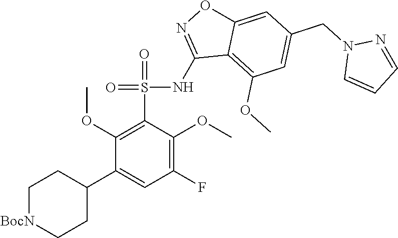 [SMILES: COc1c(F)cc(C2CCN(C(=O)OC(C)(C)C)CC2)c(OC)c1S(=O)(=O)Nc1noc2cc(Cn3cccn3)cc(OC)c12]

To a solution of N-(6-((1H-pyrazol-1-yl)methyl)-4-methoxybenzo[d]isoxazol-3-yl)-3-bromo-5-fluoro-2,6-dimethoxybenzenesulfonamide (0.10 g, 0.19 mmol) (from Example 55, Step 3) in THF (2 mL) was added CPhos Pd G3 (CAS: 1447963-73-6, 15 mg, 0.019 mmol). The reaction was sparged with nitrogen for 2 min then treated with (1-(tert-butoxycarbonyl)-piperidin-4-yl)zinc(II) iodide (0.6 M in THF, 1.23 mL, 0.739 mmol). The reaction was stirred at 50° C. for 4 h, then cooled to room temperature, and directly purified by silica gel chromatography (0-5% MeOH/DCM) to give the title compound (47 mg, 39%) as a brown oil. LCMS calc. for C₃₀H₃₇FN₅O₈S [M+H]⁺: m/z=646.2; Found: 646.2.

### Step 3. N-(6-((1H-Pyrazol-1-yl)methyl)-4-methoxybenzo[d]isoxazol-3-yl)-3-fluoro-2,6-dimethoxy-5-(piperidin-4-yl)benzenesulfonamide

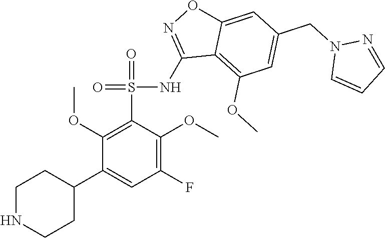 [SMILES: COc1c(F)cc(C2CCNCC2)c(OC)c1S(=O)(=O)Nc1noc2cc(Cn3cccn3)cc(OC)c12]

To a solution of tert-butyl 4-(3-(N-(6-((1H-pyrazol-1-yl)methyl)-4-methoxybenzo-[d]isoxazol-3-yl)sulfamoyl)-5-fluoro-2,4-dimethoxyphenyl)piperidine-1-carboxylate (47 mg, 0.073 mmol) in DCM (1.5 mL) was added trifluoroacetic acid (0.11 mL, 1.4 mmol). The reaction stirred for 2 h, then concentrated. The crude material was diluted with DMSO, filtered, and purified by prep-HPLC on a C18 column (21.5-41.5 CH₃CN/0.2% TFA (aq.)) to afford the title compound (21 mg, 53%) as a white TFA salt. LCMS calc. for C₂₅H₂₉FN₅O₆S [M+H]⁺: m/z=546.2; Found: 546.1.

### Step 4. N-(6-((1H-Pyrazol-1-yl)methyl)-4-methoxybenzo[d]isoxazol-3-yl)-3-(1-((1-(3-(2,4-dioxotetrahydropyrimidin-1(2H)-yl)-1-methyl-1H-indazol-6-yl)piperidin-4-yl)methyl)piperidin-4-yl)-5-fluoro-2,6-dimethoxybenzenesulfonamide

The title compound was synthesized according to procedures analogous to Example 1, Step 6 using 1-(3-(2,4-dioxotetrahydropyrimidin-1(2H)-yl)-1-methyl-1H-indazol-6-yl)piperidine-4-carbaldehyde (which can be prepared as described in WO2023249970A1). LCMS calc. for C₄₃H₅₀FN₁₀O₈S [M+H]⁺: m/z=885.3; Found: 885.3.

### Example 82. N-(6-((1H-Pyrazol-1-yl)methyl)-4-methoxybenzo[d]isoxazol-3-yl)-3-(4-((4-(3-(2,4-dioxotetrahydropyrimidin-1(2H)-yl)-1-methyl-1H-indazol-6-yl)piperidin-1-yl)methyl)cyclohexyl)-5-fluoro-2,6-dimethoxybenzenesulfonamide

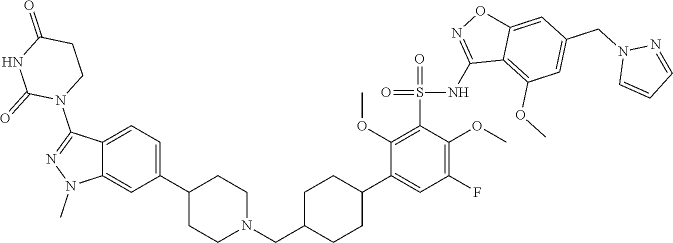 [SMILES: COc1c(F)cc(C2CCC(CN3CCC(c4ccc5c(N6CCC(=O)NC6=O)nn(C)c5c4)CC3)CC2)c(OC)c1S(=O)(=O)Nc1noc2cc(Cn3cccn3)cc(OC)c12]

### Step 1. trans-4-(((tert-Butyldiphenylsilyl)oxy)methyl)cyclohexan-1-ol

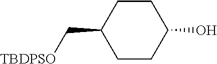

To a solution of trans-4-(hydroxymethyl)cyclohexan-1-ol (1.0 g, 7.7 mmol) and imidazole (784 mg, 11.5 mmol) in DCM (38 mL) was added tert-butylchlorodiphenylsilane (2.0 mL, 7.7 mmol) dropwise. The reaction mixture was stirred overnight then washed with 1N HCl (aq.) (100 mL). The organic layer was washed with brine (100 mL), dried over anhydrous Na₂SO₄, filtered, and concentrated. The resulting residue was purified by silica gel chromatography (0-40% EtOAc/hexanes) to afford the title compound (1.35 g, 48%) as a colorless, viscous oil. ¹H NMR (400 MHz, CDCl₃) δ 7.68 (m, 4H), 7.52 (m, 6H), 4.49 (d, J=4.4 Hz, 1H), 3.44 (d, J=6.2 Hz, 2H), 3.33 (m, 1H), 1.90 (m, 2H), 1.77 (m, 2H), 1.42 (ddp, J=11.8, 9.5, 3.1 Hz, 1H), 1.16 (m, 2H), 0.99 (s, 8H), 0.98 (m, 2H).

### Step 2. cis-tert-Butyl((-4-iodocyclohexyl)methoxy)diphenylsilane

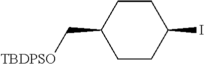

To a solution of polymer-supported PPh₃ (100-200 mesh, ~1.6 mmol/g loading; 2.74 g, 4.40 mmol) and imidazole (0.32 g, 4.8 mmol) in DCM (12 mL) was added iodine (1.12 g, 4.40 mmol) and stirred for 30 min. trans-4-(((tert-Butyldiphenylsilyl)oxy)methyl)cyclohexan-1-ol (1.35 g, 3.66 mmol) was added and the mixture was stirred overnight. The reaction mixture was filtered through a pad of Celite, concentrated, and purified by silica gel chromatography (0-20% EtOAc/hexanes) to afford the title compound (1.05 g, 59.9%) as a colorless, viscous oil. ¹H NMR (400 MHz, DMSO-d₆) δ 7.68 (m, 4H), 7.52 (m, 6H), 4.49 (d, J=4.4 Hz, 1H), 3.44 (d, J=6.2 Hz, 2H), (m, 1H), 1.90 (m, 2H), 1.77 (m, 2H), 1.42 (ddp, J=11.8, 9.5, 3.1 Hz, 1H), 1.16 (m, 2H), 0.99 (s, 8H), 0.98 (m, 2H).

### Step 3. (4-(((tert-Butyldiphenylsilyl)oxy)methyl)cyclohexyl)zinc(II) iodide

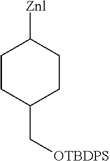

This title compound was synthesized by procedures analogues to those outlines in Example 81, Step 1, utilizing cis-tert-butyl((-4-iodocyclohexyl)methoxy)diphenylsilane instead of tert-butyl 4-iodopiperidine-1-carboxylate.

### Step 4. N-(6-((1H-Pyrazol-1-yl)methyl)-4-methoxybenzo[d]isoxazol-3-yl)-3-(4-(((tert-butyldiphenylsilyl)oxy)methyl)cyclohexyl)-5-fluoro-2,6-dimethoxybenzenesulfonamide (mixture of diastereomers)

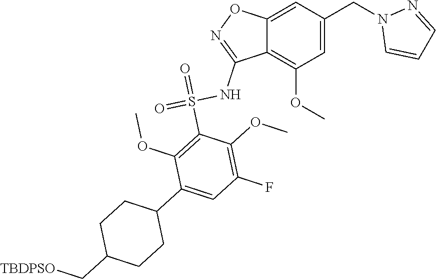

The inseparable mixture of 2 diastereomeric title compounds were synthesized by procedures analogues to those outlines in Example 81, Step 2, utilizing (4-(((tert-butyl-diphenylsilyl)oxy)methyl)cyclohexyl)zinc(II) iodide instead of (1-(tert-butoxycarbonyl)-piperidin-4-yl)zinc(II) iodide. LCMS calc. for C₄₃H₅₀FN₄O₇SSi [M+H]⁺: m/z=813.3; Found: 813.2.

### Step 5. N-(6-((1H-Pyrazol-1-yl)methyl)-4-methoxybenzo[d]isoxazol-3-yl)-3-fluoro-5-(4-(hydroxymethyl)cyclohexyl)-2,6-dimethoxybenzenesulfonamide (mixture of diastereomers)

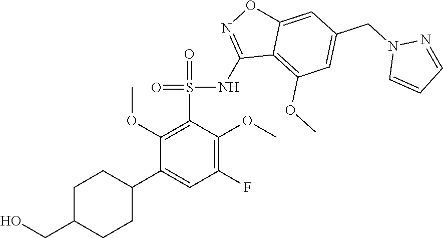 [SMILES: COc1c(F)cc(C2CCC(CO)CC2)c(OC)c1S(=O)(=O)Nc1noc2cc(Cn3cccn3)cc(OC)c12]

To a solution of N-(6-((1H-pyrazol-1-yl)methyl)-4-methoxybenzo[d]isoxazol-3-yl)-3-(4-(((tert-butyldiphenylsilyl)oxy)methyl)cyclohexyl)-5-fluoro-2,6-dimethoxybenzene-sulfonamide (80 mg, 0.15 mmol) in THF (1.3 mL) was added tetrabutylammonium fluoride (1 M in THF) (1.23 mL, 1.23 mmol). The reaction mixture was stirred at 50° C. for 5 h. The mixture was cooled to room temperature, diluted with DCM (20 mL), and washed with brine (2×20 mL). The combined organic layers were dried over anhydrous sodium sulfate, filtered, concentrated, and purified by silica gel chromatography (0-5% MeOH/DCM) to give inseparable mixture of 2 diastereomeric title compounds (69 mg, 97% yield) as a pink solid. LCMS calc. for C₂₇H₃₂FN₄O₇S [M+H]⁺: m/z=575.2; Found: 575.1.

### Step 6. N-(6-((1H-Pyrazol-1-yl)methyl)-4-methoxybenzo[d]isoxazol-3-yl)-3-fluoro-5-(4-formylcyclohexyl)-2,6-dimethoxybenzenesulfonamide

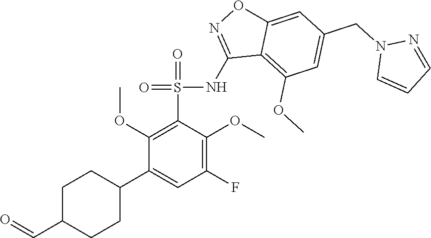 [SMILES: COc1c(F)cc(C2CCC(C=O)CC2)c(OC)c1S(=O)(=O)Nc1noc2cc(Cn3cccn3)cc(OC)c12]

To a solution of N-(6-((1H-pyrazol-1-yl)methyl)-4-methoxybenzo[d]isoxazol-3-yl)-3-fluoro-5-(4-(hydroxymethyl)cyclohexyl)-2,6-dimethoxybenzenesulfonamide (8.0 mg, 0.014 mmol) and triethylamine (0.019 mL, 0.14 mmol) in DMSO (0.50 mL) was added sulfur trioxide triethylamine complex (15 mg, 0.084 mmol) in DMSO (0.50 mL). The reaction was stirred for 2 h. The material was carried forward without further purification. LCMS calc. for C₂₇H₃₀FN₄O₇S [M+H]⁺: m/z=573.2; Found: 573.1.

### Step 7. N-(6-((1H-Pyrazol-1-yl)methyl)-4-methoxybenzo[d]isoxazol-3-yl)-3-(4-((4-(3-(2,4-dioxotetrahydropyrimidin-1(2H)-yl)-1-methyl-1H-indazol-6-yl)piperidin-1-yl)methyl)cyclohexyl)-5-fluoro-2,6-dimethoxybenzenesulfonamide (mixture of diastereomers)

To a solution of N-(6-((1H-pyrazol-1-yl)methyl)-4-methoxybenzo[d]isoxazol-3-yl)-3-fluoro-5-(4-formylcyclohexyl)-2,6-dimethoxybenzenesulfonamide (8.0 mg, 0.014 mmol) in DMSO (0.5 mL) was added a solution of 1-(1-methyl-6-(piperidin-4-yl)-1H-indazol-3-yl)dihydropyrimidine-2,4(1H,3H)-dione hydrochloride (which can be prepared as described in WO2022235945 A1) (9.1 mg, 0.028 mmol) in DMSO (0.5 mL), followed by N,N-diisopropylethylamine (0.035 mL, 0.20 mmol). The reaction was stirred for 10 min, then sodium triacetoxyborohydride (21.2 mg, 0.10 mmol) was added, followed by acetic acid (0.011 mL, 0.20 mmol). The reaction was stirred for 1 h then quenched with water, diluted with DMSO, filtered, and purified by prep-HPLC on a C18 column (21.3-41.3% CH₃CN/0.2% TFA (aq.)) to afford the title compound as a TFA salt (1.8 mg, 13% yield), a white solid. LCMS calc. for C₄₄H₅₁FN₉O₈S [M+H]⁺: m/z=884.4; Found: 884.4.

### Examples 83-84

Examples 83-84 listed in Table 4 were synthesized according to procedures analogous to Example 81 using appropriate starting materials. All examples in these tables were prepared as the TFA salt unless otherwise noted.

TABLE 4
Examples 83-84

| Ex- | | LCMS |
|---|---|---|
| ample | Structure/Name | [M + H]⁺ |
| 83 | 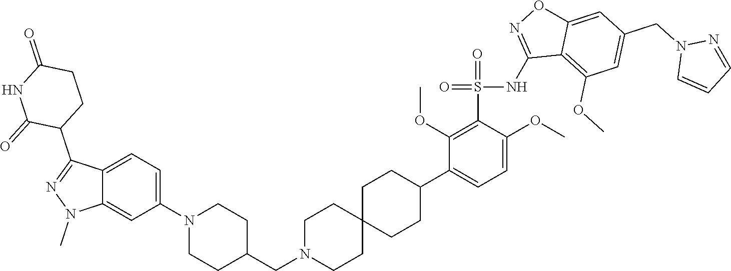 [SMILES: COc1ccc(C2CCC3(CC2)CCN(CC2CCN(c4ccc5c(C6CCC(=O)NC6=O)nn(C)c5c4)CC2)CC3)c(OC)c1S(=O)(=O)Nc1noc2cc(Cn3cccn3)cc(OC)c12] | 934.4 |
| | N-(6-((1H-Pyrazol-1-yl)methyl)-4- | |
| | methoxybenzo[d]isoxazol-3-yl)-3-(3-((1-(3-(2,6- | |
| | dioxopiperidin-3-yl)-1-methyl-1H-indazol-6-yl)piperidin- | |
| | 4-yl)methyl)-3-azaspiro[5.5]undecan-9-yl)-2,6- | |
| | dimethoxybenzenesulfonamide | |
| | ¹H NMR (400 MHz, CD₃OD) δ 7.79 (d, J = 2.4 Hz, 1H), | |
| | 7.76 (m, 1H), 7.63 (m, 1H), 7.53 (d, J = 8.8 Hz, 1H), 7.33 | |
| | (m, 2H), 6.90 (d, J = 8.9 Hz, 1H), 6.78 (d, J = 1.4 Hz, 1H), | |
| | 6.70 (s, 1H), 6.39 (q, J = 1.8 Hz, 1H), 5.48 (s, 2H), 4.47 | |
| | (m, 1H), 4.01 (s, 6H), 3.94 (s, 3H), 3.91 (m, 5H), 3.60 (m, | |
| | 2H), 3.19 (p, J = 11.0 Hz, 6H), 3.02 (m, 1H), 2.86 (m, | |
| | 2H), 2.58 (m, 5H), 2.08 (dt, J = 15.2, 4.2 Hz, 2H), 1.92 | |
| | (m, 1H), 1.76 (m, 11H), 1.39-1.23 (m, 1H). | |
| 84 | 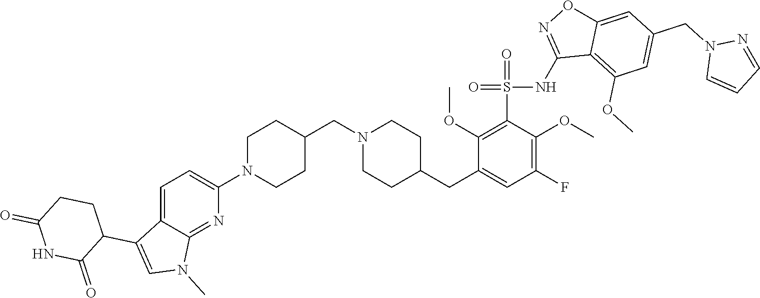 [SMILES: COc1c(F)cc(CC2CCN(CC3CCN(c4ccc5c(C6CCC(=O)NC6=O)cn(C)c5n4)CC3)CC2)c(OC)c1S(=O)(=O)Nc1noc2cc(Cn3cccn3)cc(OC)c12] | 898.2 |
| | N-(6-((1H-Pyrazol-1-yl)methyl)-4- | |
| | methoxybenzo[d]isoxazol-3-yl)-3-((1-((1-(3-(2,6- | |
| | dioxopiperidin-3-yl)-1-methyl-1H-pyrrolo[2,3-b]pyridin- | |
| | 6-yl)piperidin-4-yl)methyl)piperidin-4-yl)methyl)-5- | |
| | fluoro-2,6-dimethoxybenzenesulfonamide | |

### Example 85. N-(6-((1H-Pyrazol-1-yl)methyl)-4-methoxybenzo[d]isoxazol-3-yl)-4′-((4-(3-(2,4-dioxotetrahydropyrimidin-1(2H)-yl)-1-methyl-1H-indazol-6-yl)piperidin-1-yl)methyl)-2,4-dimethoxy-[1,1′-biphenyl]-3-sulfonamide

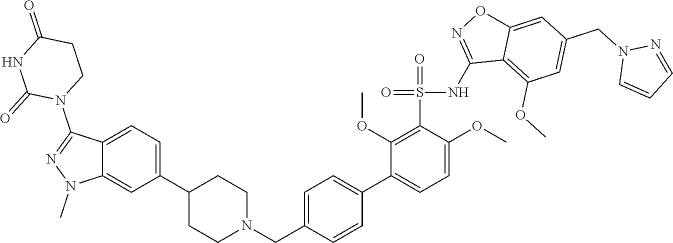 [SMILES: COc1ccc(-c2ccc(CN3CCC(c4ccc5c(N6CCC(=O)NC6=O)nn(C)c5c4)CC3)cc2)c(OC)c1S(=O)(=O)Nc1noc2cc(Cn3cccn3)cc(OC)c12]

### Step 1: N-(6-((1H-Pyrazol-1-yl)methyl)-4-methoxybenzo[d]isoxazol-3-yl)-4′-formyl-2,4-dimethoxy-[1,1′-biphenyl]-3-sulfonamide

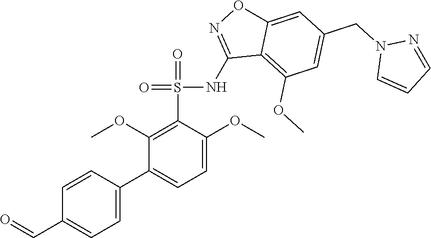 [SMILES: COc1ccc(-c2ccc(C=O)cc2)c(OC)c1S(=O)(=O)Nc1noc2cc(Cn3cccn3)cc(OC)c12]

To a nitrogen sparged solution of N-(6-((1H-pyrazol-1-yl)methyl)-4-methoxybenzo-[d]isoxazol-3-yl)-3-bromo-2,6-dimethoxybenzenesulfonamide (120 mg, 0.229 mmol) (which can be prepared as described in WO2022235945 A1) in water (0.40 mL) and 1,4-dioxane (1.9 mL) was added potassium carbonate (95.1 mg, 0.688 mmol), SPhos Pd G2 (CAS: 1375325-64-6, 16.5 mg, 0.0229 mmol), and (4-formylphenyl)boronic acid (85.9 mg, 0.573 mmol). The mixture was stirred at 90° C. for 90 minutes, under a nitrogen atmosphere. After cooling to room temperature, the mixture was filtered through Celite, concentrated, and purified by silica gel chromatography (0-90% EtOAc/hexanes to 0-5% MeOH/DCM) to afford the title compound (90 mg, 72%) as a pale yellow solid. LCMS calc. for C₂₇H₂₅N₄O₇S [M+H]⁺: m/z=549.1; Found: 549.1.

### Step 2: N-(6-((1H-Pyrazol-1-yl)methyl)-4-methoxybenzo[d]isoxazol-3-yl)-4′-((4-(3-(2,4-dioxotetrahydropyrimidin-1(2H)-yl)-1-methyl-1H-indazol-6-yl)piperidin-1-yl)methyl)-2,4-dimethoxy-[1,1′-biphenyl]-3-sulfonamide

The title compound was synthesized by procedures analogous to that described in Example 82, Step 5. ¹H NMR (400 MHz, CD₃OD) δ 7.77 (d, J=2.4 Hz, 1H), 7.69 (m, 5H), 7.56 (m, 2H), 7.35 (s, 1H), 7.12 (m, 1H), 6.99 (d, J=8.8 Hz, 1H), 6.73 (d, J=28.2 Hz, 2H), 6.36 (t, J=2.2 Hz, 1H), 5.47 (s, 2H), 4.42 (s, 2H), 4.16 (m, 8H), 3.84 (s, 3H), 3.76 (m, 2H), 3.48 (s, 3H), 3.27 (m, 2H), 3.14 (m, 1H), 2.85 (t, J=6.7 Hz, 2H), 2.35 (m, 2H), 2.14 (m, 2H). LCMS calc. for C₄₄H₄₆N₉O₈S [M+H]⁺: m/z=860.3; Found: 860.8.

### Example 86-87

Examples 86-87 listed in Table 5 were synthesized according to procedures analogous to Example 85 using appropriate starting materials. All examples in these tables were prepared as the TFA salt unless otherwise noted.

TABLE 5
Examples 86-87

| | | LCMS |
|---|---|---|
| Example VD | Structure/Name | [M + H]⁺ |
| 86 | 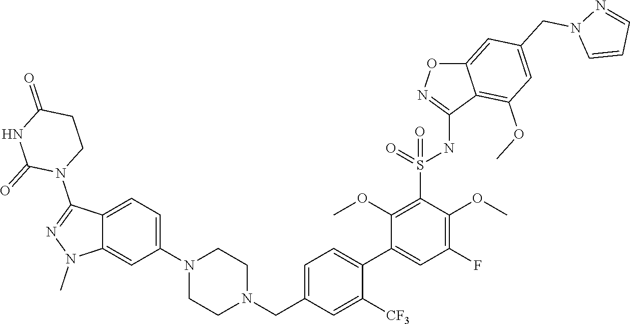 [SMILES: COc1c(F)cc(-c2ccc(CN3CCN(c4ccc5c(N6CCC(=O)NC6=O)nn(C)c5c4)CC3)cc2C(F)(F)F)c(OC)c1S(=O)(=O)Nc1noc2cc(Cn3cccn3)cc(OC)c12] | 947.2 |
| | N-(6-((1H-Pyrazol-1-yl)methyl)-4- | |
| | methoxybenzo[d]isoxazol-3-yl)-4′-((4-(3-(2,4- | |
| | dioxotetrahydropyrimidin-1(2H)-yl)-1-methyl-1H- | |
| | indazol-6-yl)piperazin-1-yl)methyl)-5-fluoro-2,4- | |
| | dimethoxy-2′-(trifluoromethyl)-[1,1′-biphenyl]-3- | |
| | sulfonamide | |
| 87 | 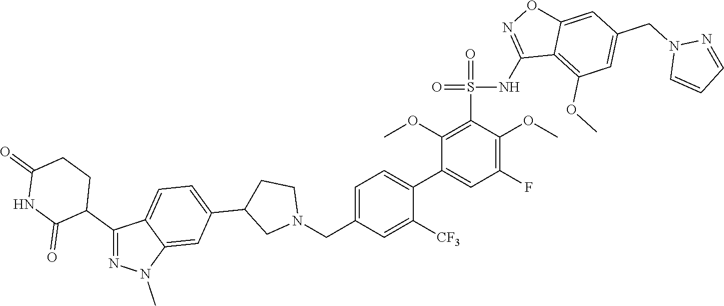 [SMILES: COc1c(F)cc(-c2ccc(CN3CCC(c4ccc5c(C6CCC(=O)NC6=O)nn(C)c5c4)C3)cc2C(F)(F)F)c(OC)c1S(=O)(=O)Nc1noc2cc(Cn3cccn3)cc(OC)c12] | 931.4 |
| | N-(6-((1H-Pyrazol-1-yl)methyl)-4- | |
| | methoxybenzo[d]isoxazol-3-yl)-4′-((3-(3-(2,6- | |
| | dioxopiperidin-3-yl)-1-methyl-1H-indazol-6- | |
| | yl)pyrrolidin-1-yl)methyl)-5-fluoro-2,4-dimethoxy- | |
| | 2′-(trifluoromethyl)-[1,1′-biphenyl]-3-sulfonamide | |

### Example 88. rac-N-(6-((1H-Pyrazol-1-yl)methyl)-4-methoxybenzo[d]isoxazol-3-yl)-3-((3a,7a)-5-((1-(3-(2,4-dioxotetrahydropyrimidin-1(2H)-yl)-1-methyl-1H-indazol-6-yl)piperidin-4-yl)methyl)octahydro-2H-pyrrolo[3,4-c]pyridin-2-yl)-5-fluoro-2,6-dimethoxybenzenesulfonamide

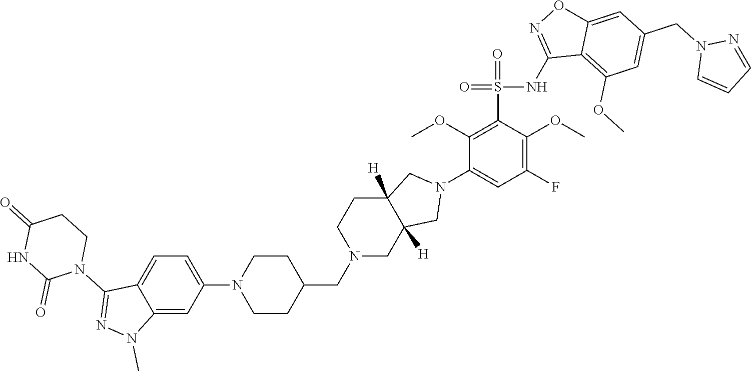 [SMILES: COc1c(F)cc(N2C[C@H]3CCN(CC4CCN(c5ccc6c(N7CCC(=O)NC7=O)nn(C)c6c5)CC4)C[C@H]3C2)c(OC)c1S(=O)(=O)Nc1noc2cc(Cn3cccn3)cc(OC)c12]

### Step 1. rac-tert-Butyl (3a, 7a)-2-(3-(N-(6-((1H-pyrazol-1-yl)methyl)-4-methoxybenzo-[d]isoxazol-3-yl)sulfamoyl)-5-fluoro-2,4-dimethoxyphenyl)octahydro-5H-pyrrolo[3,4-c]pyridine-5-carboxylate

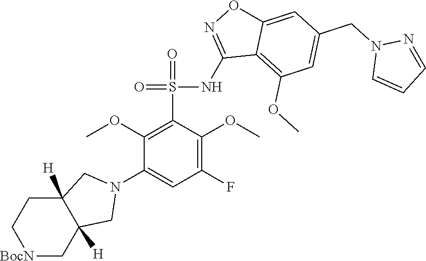 [SMILES: COc1c(F)cc(N2C[C@H]3CCN(C(=O)OC(C)(C)C)C[C@H]3C2)c(OC)c1S(=O)(=O)Nc1noc2cc(Cn3cccn3)cc(OC)c12]

A nitrogen sparged solution of rac-tert-butyl (3a,7a)-octahydro-5H-pyrrolo[3,4-c]-pyridine-5-carboxylate hydrochloride (CAS: 1257389-94-8, 100 mg, 0.185 mmol), cesium carbonate (181 mg, 0.554 mmol), N-(6-((1H-pyrazol-1-yl)methyl)-4-methoxybenzo[d]-isoxazol-3-yl)-3-bromo-5-fluoro-2,6-dimethoxybenzenesulfonamide (100 mg, 0.443 mmol, Example 55, Step 3), and Pd-PEPPSI-iHeptCl (CAS: 1814936-54-3, 18.0 mg, 0.0185 mmol) in 1,4-dioxane (1.8 mL) was stirred at 100° C. for 2 h. After cooling to room temperature, the mixture was diluted with DCM (20 mL), filtered through a pad of Celite, dried over MgSO₄, and concentrated. The crude material was purified by silica gel chromatography (0-5% MeOH/DCM) to give the racemic title compound (15 mg, 12%) as a thick yellow oil. LCMS calc. for C₃₂H₄₀FN₆O₈S [M+H]⁺: m/z=687.3; Found: 687.2.

### Step 2. rac-N-(6-((1H-Pyrazol-1-yl)methyl)-4-methoxybenzo[d]isoxazol-3-yl)-3-((3a, 7a)-5-((1-(3-(2,4-dioxotetrahydropyrimidin-1(2H)-yl)-1-methyl-1H-indazol-6-yl)piperidin-4-yl)methyl)octahydro-2H-pyrrolo[3,4-c]pyridin-2-yl)-5-fluoro-2,6-dimethoxy-benzenesulfonamide

The racemic title compound was synthesized by procedures analogous to those described in Example 81, Steps 3-4. LCMS calc. for C₄₅H₅₃FN₁₁O₈S [M+H]⁺: m/z=926.4; Found: 926.6.

### Example 89-91 and 118

Examples 89-91 and 118 shown in Table 6 were synthesized according to procedures analogous to Example 88 using appropriate starting materials. All examples in this table were prepared as the TFA salt unless otherwise noted.

TABLE 6
Examples 89-91 and 118

| Exam- | | LCMS |
|---|---|---|
| ple | Structure/Name | [M + H]⁺ |
| 89 | 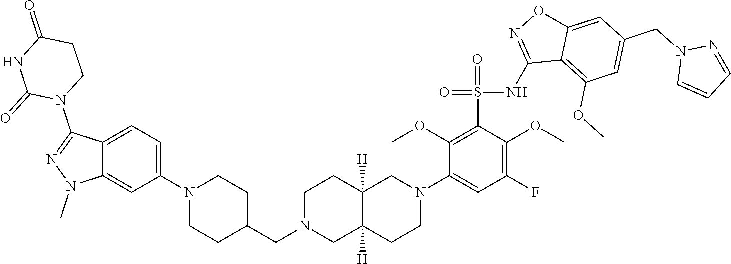 [SMILES: COc1c(F)cc(N2CC[C@H]3CN(CC4CCN(c5ccc6c(N7CCC(=O)NC7=O)nn(C)c6c5)CC4)CC[C@H]3C2)c(OC)c1S(=O)(=O)Nc1noc2cc(Cn3cccn3)cc(OC)c12] | 940.3 |
| | N-(6-((1H-Pyrazol-1-yl)methyl)-4- | |
| | methoxybenzo[d]isoxazol-3-yl)-3-((4aR,8aR)-6-((1-(3- | |
| | (2,4-dioxotetrahydropyrimidin-1(2H)-yl)-1-methyl-1H- | |
| | indazol-6-yl)piperidin-4-yl)methyl)octahydro-2,6- | |
| | naphthyridin-2(1H)-yl)-5-fluoro-2,6- | |
| | dimethoxybenzenesulfonamide | |
| 90 | 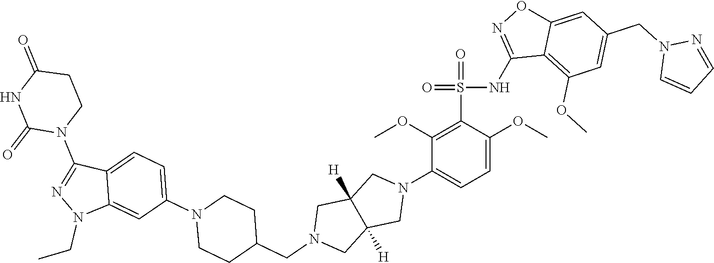 [SMILES: CCn1nc(N2CCC(=O)NC2=O)c2ccc(N3CCC(CN4C[C@@H]5CN(c6ccc(OC)c(S(=O)(=O)Nc7noc8cc(Cn9cccn9)cc(OC)c78)c6OC)C[C@H]5C4)CC3)cc21] | 907.3 |
| | N-(6-((1H-Pyrazol-1-yl)methyl)-4- | |
| | methoxybenzo[d]isoxazol-3-yl)-3-((3aR,6aR)-5-((1-(3- | |
| | (2,4-dioxotetrahydropyrimidin-1(2H)-yl)-1-ethyl-1H- | |
| | indazol-6-yl)piperidin-4-yl)methyl)hexahydropyrrolo[3,4- | |
| | c]pyrrol-2(1H)-yl)-2,6-dimethoxybenzenesulfonamide | |
| 91 | 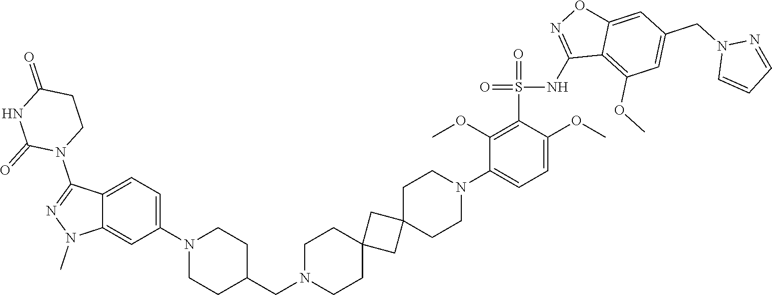 [SMILES: COc1ccc(N2CCC3(CC2)CC2(CCN(CC4CCN(c5ccc6c(N7CCC(=O)NC7=O)nn(C)c6c5)CC4)CC2)C3)c(OC)c1S(=O)(=O)Nc1noc2cc(Cn3cccn3)cc(OC)c12] | 977.3 |
| | N-(6-((1H-Pyrazol-1-yl)methyl)-4- | |
| | methoxybenzo[d]isoxazol-3-yl)-3-(11-((1-(3-(2,4- | |
| | dioxotetrahydropyrimidin-1(2H)-yl)-1-methyl-1H- | |
| | indazol-6-yl)piperidin-4-yl)methyl)-3,11- | |
| | diazadispiro[5.1.58.16]tetradecan-3-yl)-2,6- | |
| | dimethoxybenzenesulfonamide | |
| 118 | 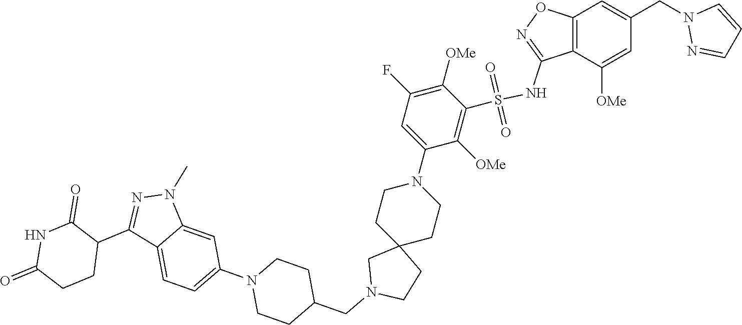 [SMILES: COc1c(F)cc(N2CCC3(CCN(CC4CCN(c5ccc6c(C7CCC(=O)NC7=O)nn(C)c6c5)CC4)C3)CC2)c(OC)c1S(=O)(=O)Nc1noc2cc(Cn3cccn3)cc(OC)c12] | 940.1 |
| | N-(6-((1H-Pyrazol-1-yl)methyl)-4- | |
| | methoxybenzo[d]isoxazol-3-yl)-3-(2-((1-(3-(2,6- | |
| | dioxopiperidin-3-yl)-1-methyl-1H-indazol-6-yl)piperidin- | |
| | 4-yl)methyl)-2,8-diazaspiro[4.5]decan-8-yl)-5-fluoro-2,6- | |
| | dimethoxybenzenesulfonamide | |
| | ¹H NMR (300 MHz, DMSO-d₆) δ 10.88 (s, 1H), 10.39 (s, | |
| | 1H), 9.67 (s, 1H), 7.89 (d, J = 2.0 Hz, 1H), 7.53 (dd, J = | |
| | 11.7, 5.4 Hz, 2H), 7.23 (d, J = 13.1 Hz, 1H), 6.99 (d, J = | |
| | 7.4 Hz, 2H), 6.86 (s, 1H), 6.75 (s, 1H), 6.31 (t, J = 2.0 Hz, | |
| | 1H), 5.46 (s, 2H), 4.28 (dd, J = 9.2, 5.1 Hz, 1H), 3.91 (s, | |
| | 3H), 3.85 (s, 1H), 3.80 (d, J = 5.5 Hz, 6H), 3.77 (s, 3H), | |
| | 3.70 (s, 1H), 3.64-3.56 (m, 1H), 3.17 (s, 3H), 3.02-2.77 | |
| | (m, 7H), 2.63 (dd, J = 11.6, 6.8 Hz, 2H), 2.37-2.26 (m, | |
| | 1H), 2.16 (dd, J = 13.4, 5.5 Hz, 1H), 2.07-1.68 (m, 9H), | |
| | 1.41 (d, J = 10.8 Hz, 2H). | |

### Example 92. N-(6-((1H-Pyrazol-1-yl)methyl)-4-methoxybenzo[d]isoxazol-3-yl)-3-(7-((1-(3-(2,4-dioxotetrahydropyrimidin-1(2H)-yl)-1-ethyl-1H-indazol-6-yl)piperidin-4-yl)methyl)-2,7-diazaspiro[3.5]nonan-2-yl)-5-fluoro-2,6-dimethoxybenzenesulfonamide

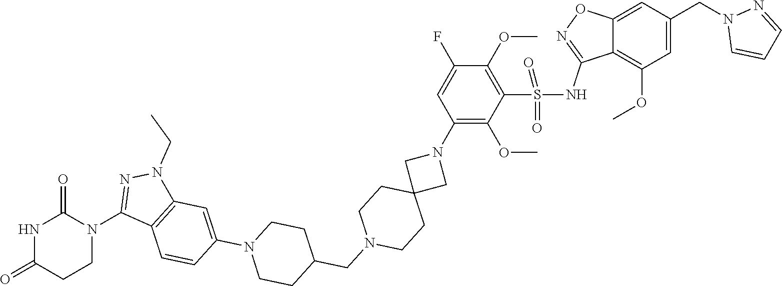 [SMILES: CCn1nc(N2CCC(=O)NC2=O)c2ccc(N3CCC(CN4CCC5(CC4)CN(c4cc(F)c(OC)c(S(=O)(=O)Nc6noc7cc(Cn8cccn8)cc(OC)c67)c4OC)C5)CC3)cc21]

### Step 1. 6-Bromo-1-ethyl-3-iodo-1H-indazole

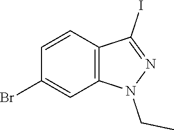

To a solution of 6-bromo-3-iodo-1H-indazole (4.00 g, 12.4 mmol) in DMF (30 mL) was added potassium hydroxide (1.04 g, 18.6 mmol) at 0° C. Diethyl sulfate (2.29 g, 14.9 mmol) was added to the mixture dropwise. The reaction mixture was stirred at room temperature for 2 h, then poured into water and extracted with ethyl acetate. The combined organic layers were washed with brine, dried over Na₂SO₄, filtered, and concentrated. The crude material was purified by silica column chromatography (0-50% EtOAc/heptane) to afford the title compound (3.52 g, 10.0 mmol, 81.0% yield) as a light brown solid. LCMS calc. for C₉H₉BrIN₂ [M+H]⁺ m/z=350.9, 352.9; Found 350.9, 352.9.

### Step 2. 1-(6-Bromo-1-ethyl-1H-indazol-3-yl)-3-(4-methoxybenzyl)dihydro-pyrimidine-2,4(1H,3H)-dione

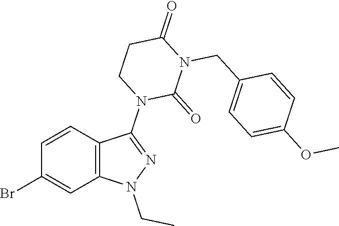 [SMILES: CCn1nc(N2CCC(=O)N(Cc3ccc(OC)cc3)C2=O)c2ccc(Br)cc21]

To a solution of 6-bromo-1-ethyl-3-iodo-1H-indazole (2.32 g, 6.61 mmol) in DMSO (30 mL) was added 3-(4-methoxybenzyl)dihydropyrimidine-2,4(1H,3H)-dione (2.32 g, 9.91 mmol), K₃PO₄ (2.81 g, 13.2 mmol), 3,4,7,8-tetramethyl-1,10-phenanthroline (312 mg, 1.32 mmol), and Cu(OAc)₂ (120 mg, 0.661 mmol). The reaction was stirred at 80° C. for 16 h. The mixture was poured into water and extracted with EtOAc. The combined organic layers were washed with brine, dried over Na₂SO₄, filtered, and concentrated. The crude material was purified by silica column chromatography (0-90% EtOAc/heptane) to afford the title compound (2.05 g, 4.48 mmol, 67.8% yield) as a yellow solid. LCMS calc. for C₂₁H₂₂BrN₄O₃ [M+H]⁺ m/z=457.1, 459.1; Found 457.2, 459.2.

### Step 3. 1-(6-Bromo-1-ethylindazol-3-yl)-1,3-diazinane-2,4-dione

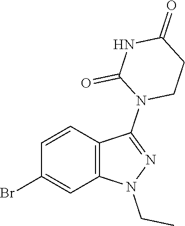 [SMILES: CCn1nc(N2CCC(=O)NC2=O)c2ccc(Br)cc21]

A mixture of TFA (4.9 g, 3.3 mL, 43 mmol), trifluoromethanesulfonic acid (1.2 g, 0.70 mL, 8.0 mmol) and 1-(6-bromo-1-ethylindazol-3-yl)-1,3-diazinane-2,4-dione (2.1 g, 4.5 mmol) was stirred at 70° C. for 2 h. The reaction was quenched by sat. NaHCO₃ (aq.). The mixture was extracted by EtOAc. The combined organic layers were washed with brine, dried over Na₂SO₄, filtered, and concentrated. The crude material was purified by silica column chromatography (0-100% EtOAc/DCM) to afford the title compound (1.46 g, 4.33 mmol, 96.7% yield) as an orange solid. LCMS calc. for C₁₃H₁₄BrN₄O₂ [M+H]⁺ m/z=337.0, 339.0; Found 337.2, 339.2.

### Step 4. 1-(6-(4-(Dimethoxymethyl)piperidin-1-yl)-1-ethyl-1H-indazol-3-yl)dihydro-pyrimidine-2,4(1H,3H)-dione

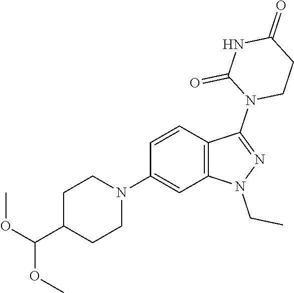 [SMILES: CCn1nc(N2CCC(=O)NC2=O)c2ccc(N3CCC(C(OC)OC)CC3)cc21]

The title compound was synthesized by procedures analogous to that described in Example 4, Step 1. LCMS calc. for C₂₁H₃₀N₅O₄ [M+H]⁺: m/z=416.2; found: 416.3.

### Step 5. 1-(3-(2,4-Dioxotetrahydropyrimidin-1(2H)-yl)-1-ethyl-1H-indazol-6-yl)piperidine-4-carbaldehyde

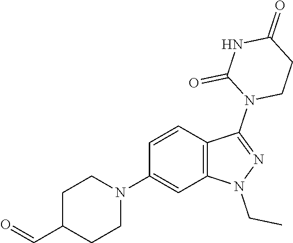 [SMILES: CCn1nc(N2CCC(=O)NC2=O)c2ccc(N3CCC(C=O)CC3)cc21]

The title compound was synthesized by procedures analogous to that described in Example 4, Step 4. LCMS calc. for C₁₉H₂₄N₅O₃ [M+H]⁺: m/z=370.2; Found: 370.1.

### Step 6. N-(6-((1H-Pyrazol-1-yl)methyl)-4-methoxybenzo[d]isoxazol-3-yl)-3-(7-((1-(3-(2,4-dioxotetrahydropyrimidin-1(2H)-yl)-1-ethyl-1H-indazol-6-yl)piperidin-4-yl)methyl)-2, 7-diazaspiro[3.5]nonan-2-yl)-5-fluoro-2,6-dimethoxybenzenesulfonamide

The title compound was synthesized by procedures analogous to that described in Example 2, using N-(6-((1H-pyrazol-1-yl)methyl)-4-methoxybenzo[d]isoxazol-3-yl)-3-fluoro-2,6-dimethoxy-5-(2,7-diazaspiro[3.5]nonan-2-yl)benzenesulfonamide (from Example 58, Step 4). LCMS calc. for C₄₆H₅₅FN₁₁O₈S [M+H]⁺: m/z=940.4; Found: 940.5.

### Example 93. N-(6-((1H-Pyrazol-1-yl)methyl)-4-methoxybenzo[d]isoxazol-3-yl)-3-(7-((1-(4-(2,6-dioxopiperidin-3-yl)-2-methylphenyl)piperidin-4-yl)methyl)-2,7-diazaspiro[3.5]-nonan-2-yl)-5-fluoro-2,6-dimethoxybenzenesulfonamide

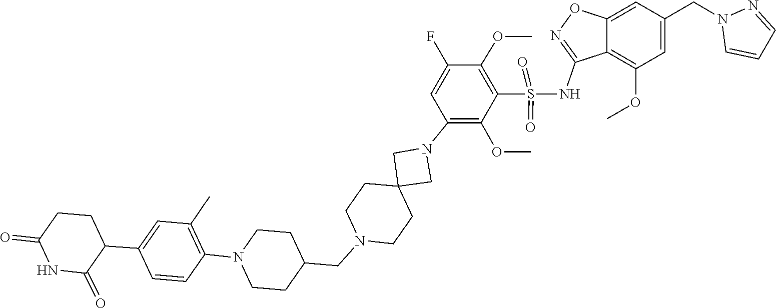 [SMILES: COc1c(F)cc(N2CC3(CCN(CC4CCN(c5ccc(C6CCC(=O)NC6=O)cc5C)CC4)CC3)C2)c(OC)c1S(=O)(=O)Nc1noc2cc(Cn3cccn3)cc(OC)c12]

The title compound was synthesized by procedures analogous to that described in Example 3, using 4-piperidinemethanol, 5-bromo-2-iodotoluene, and N-(6-((1H-pyrazol-1-yl)methyl)-4-methoxybenzo[d]isoxazol-3-yl)-3-fluoro-2,6-dimethoxy-5-(2,7-diazaspiro[3.5]-nonan-2-yl)benzenesulfonamide (from Example 58, Step 4). LCMS calc. for C₄₅H₅₄FN₈O₈S [M+H]⁺: m/z=885.4; Found: 885.5.

### Example 94. N-(6-((1H-Pyrazol-1-yl)methyl)-4-methoxybenzo[d]isoxazol-3-yl)-3-(7-((1-(4-(2,6-dioxopiperidin-3-yl)-3-fluorophenyl)piperidin-4-yl)methyl)-2,7-diazaspiro[3.5]-nonan-2-yl)-2-methoxybenzenesulfonamide

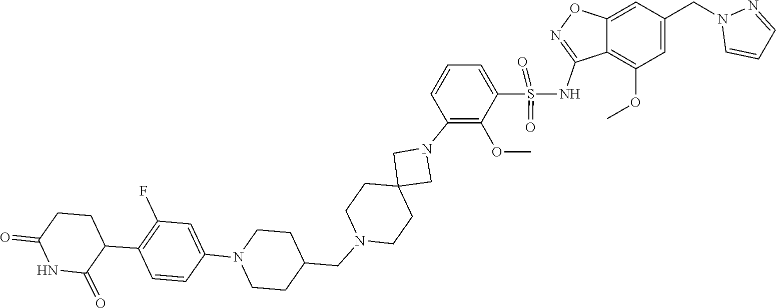 [SMILES: COc1c(N2CC3(CCN(CC4CCN(c5ccc(C6CCC(=O)NC6=O)c(F)c5)CC4)CC3)C2)cccc1S(=O)(=O)Nc1noc2cc(Cn3cccn3)cc(OC)c12]

The title compound was synthesized by procedures analogous to that described in Example 3, using 3-fluoro-4-iodobromobenzene. LCMS calc. for C₄₃H₅₀FN₈O₇S [M+H]⁺: m/z=841.3; Found: 841.4.

### Example 95. N-(6-((1H-Pyrazol-1-yl)methyl)-4-methoxybenzo[d]isoxazol-3-yl)-3-(7-((1-(4-(2,6-dioxopiperidin-3-yl)-3,5-difluorophenyl)piperidin-4-yl)methyl)-2,7-diazaspiro-[3.5]nonan-2-yl)-2-methoxybenzenesulfonamide

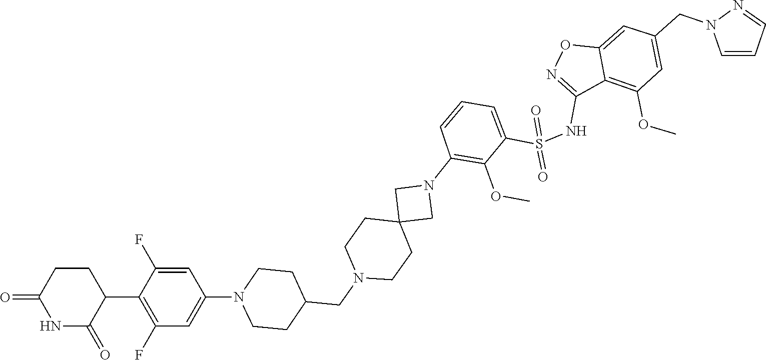 [SMILES: COc1c(N2CC3(CCN(CC4CCN(c5cc(F)c(C6CCC(=O)NC6=O)c(F)c5)CC4)CC3)C2)cccc1S(=O)(=O)Nc1noc2cc(Cn3cccn3)cc(OC)c12]

The title compound was synthesized by procedures analogous to that described in Example 1, Step 6, using 3-(2,6-difluoro-4-(4-(hydroxymethyl)piperidin-1-yl)phenyl)-piperidine-2,6-dione (CAS: 2960325-22-6) and N-(6-((1H-pyrazol-1-yl)methyl)-4-methoxy-benzo[d]isoxazol-3-yl)-2-methoxy-3-(2,7-diazaspiro[3.5]nonan-2-yl)benzenesulfonamide (which can be prepared as described in WO2023245150 A1). LCMS calc. for C₄₃H₄₉F₂N₈O₇S [M+H]⁺: m/z=859.3; Found: 859.4.

### Example 96. N-(6-((1H-Pyrazol-1-yl)methyl)-4-methoxybenzo[d]isoxazol-3-yl)-3-(7-(2-(1-(4-(2,6-dioxopiperidin-3-yl)phenyl)piperidin-4-yl)ethyl)-2,7-diazaspiro[3.5]nonan-2-yl)-5-fluoro-2,6-dimethoxybenzenesulfonamide

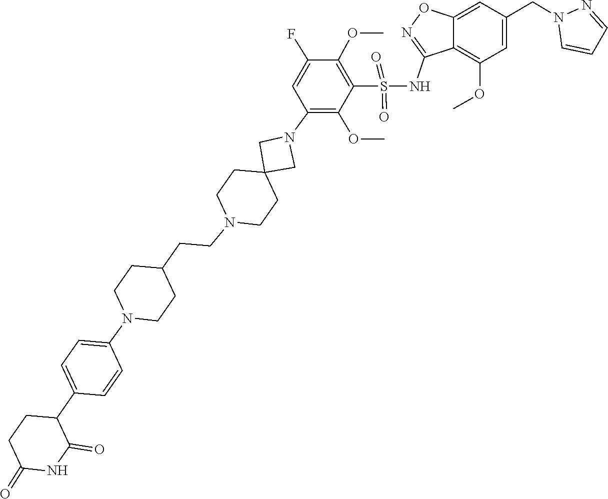 [SMILES: COc1c(F)cc(N2CC3(CCN(CCC4CCN(c5ccc(C6CCC(=O)NC6=O)cc5)CC4)CC3)C2)c(OC)c1S(=O)(=O)Nc1noc2cc(Cn3cccn3)cc(OC)c12]

The title compound was synthesized by procedures analogous to that described in Example 2, using 2-(1-(4-(2,6-dioxopiperidin-3-yl)phenyl)piperidin-4-yl)acetaldehyde (CAS: 2736510-47-5) and N-(6-((1H-pyrazol-1-yl)methyl)-4-methoxybenzo[d]isoxazol-3-yl)-3-fluoro-2,6-dimethoxy-5-(2,7-diazaspiro[3.5]nonan-2-yl)benzenesulfonamide (from Example 58, Step 4). LCMS calc. for C₄₅H₅₄FN₈O₈S [M+H]⁺: m/z=885.4; Found: 885.5.

### Example 97. N-(6-((1H-Pyrazol-1-yl)methyl)-4-methoxybenzo[d]isoxazol-3-yl)-3-(7-(((3S)-1-(1-(2,6-dioxopiperidin-3-yl)-3-methyl-1H-indazol-5-yl)pyrrolidin-3-yl)methyl)-2,7-diazaspiro[3.5]nonan-2-yl)-5-fluoro-2,6-dimethoxybenzenesulfonamide

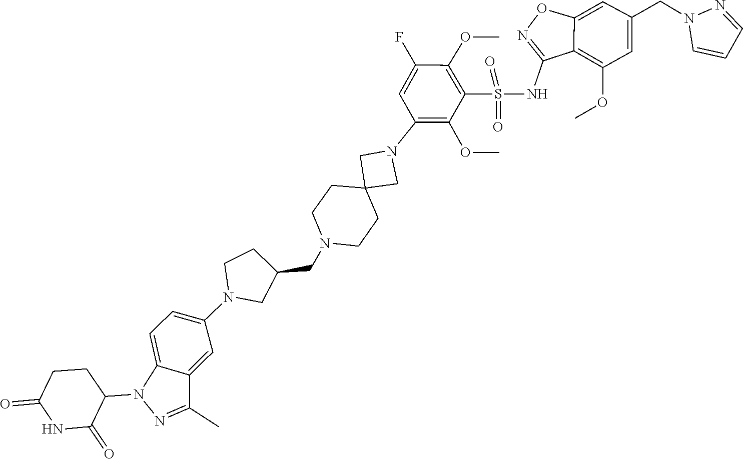 [SMILES: COc1c(F)cc(N2CC3(CCN(C[C@@H]4CCN(c5ccc6c(c5)c(C)nn6C5CCC(=O)NC5=O)C4)CC3)C2)c(OC)c1S(=O)(=O)Nc1noc2cc(Cn3cccn3)cc(OC)c12]

The title compound was synthesized by procedures analogous to that described in Example 1, using N-(6-((1H-pyrazol-1-yl)methyl)-4-methoxybenzo[d]isoxazol-3-yl)-3-fluoro-2,6-dimethoxy-5-(2,7-diazaspiro[3.5]nonan-2-yl)benzenesulfonamide (from Example 58, Step 4). LCMS calc. for C₄₅H₅₂FN₁₀O₈S [M+H]⁺: m/z=911.4; Found: 911.5.

### Example 98. N-(6-((1H-Pyrazol-1-yl)methyl)-4-methoxybenzo[d]isoxazol-3-yl)-3-(7-(((3S)-1-(4-(2,6-dioxopiperidin-3-yl)phenyl)pyrrolidin-3-yl)methyl)-2,7-diazaspiro[3.5]-nonan-2-yl)-5-fluoro-2,6-dimethoxybenzenesulfonamide

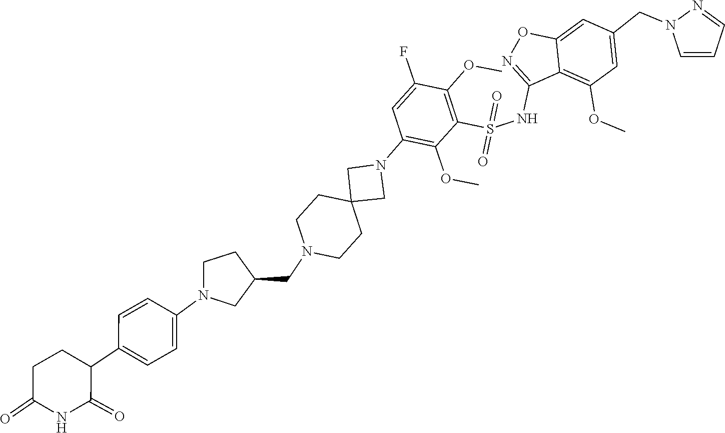 [SMILES: COc1c(F)cc(N2CC3(CCN(C[C@@H]4CCN(c5ccc(C6CCC(=O)NC6=O)cc5)C4)CC3)C2)c(OC)c1S(=O)(=O)Nc1noc2cc(Cn3cccn3)cc(OC)c12]

The title compound was synthesized by procedures analogous to that described in Example 3, using (3R)-3-pyrrolidinylmethanol and N-(6-((1H-pyrazol-1-yl)methyl)-4-methoxybenzo[d]isoxazol-3-yl)-3-fluoro-2,6-dimethoxy-5-(2,7-diazaspiro[3.5]nonan-2-yl)benzenesulfonamide (from Example 58, Step 4). LCMS calc. for C₄₃H₅₀FN₈O₈S [M+H]⁺: m/z=857.3; Found: 857.3.

### Example 99. N-(6-((1H-Pyrazol-1-yl)methyl)-4-methoxybenzo[d]isoxazol-3-yl)-3-(7-(((3R,4S)-1-(4-(2,6-dioxopiperidin-3-yl)phenyl)-3-fluoropiperidin-4-yl)methyl)-2,7-diazaspiro[3.5]nonan-2-yl)-5-fluoro-2,6-dimethoxybenzenesulfonamide

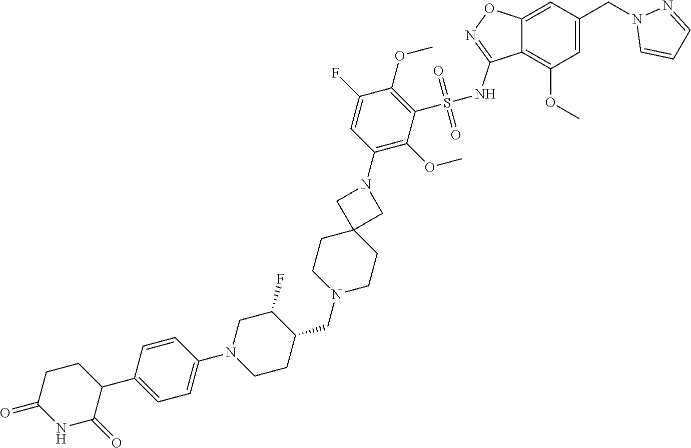 [SMILES: COc1c(F)cc(N2CC3(CCN(C[C@@H]4CCN(c5ccc(C6CCC(=O)NC6=O)cc5)C[C@@H]4F)CC3)C2)c(OC)c1S(=O)(=O)Nc1noc2cc(Cn3cccn3)cc(OC)c12]

The title compound was synthesized by procedures analogous to that described in Example 3, using [(3S,4R)-rel-3-fluoro-4-piperidyl]methanol hydrochloride and N-(6-((1H-pyrazol-1-yl)methyl)-4-methoxybenzo[d]isoxazol-3-yl)-3-fluoro-2,6-dimethoxy-5-(2,7-diazaspiro[3.5]nonan-2-yl)benzenesulfonamide (from Example 58, Step 4). LCMS calc. for C₄₄H₅₁F₂N₈O₈S [M+H]⁺: m/z=889.3; Found: 889.4.

### Example 100. N-(6-((1H-Pyrazol-1-yl)methyl)-4-methoxybenzo[d]isoxazol-3-yl)-3-(7-((1-(3-(2,4-dioxotetrahydropyrimidin-1(2H)-yl)-1-methyl-1H-pyrrolo[2,3-b]pyridin-6-yl)-4-fluoropiperidin-4-yl)methyl)-2,7-diazaspiro[3.5]nonan-2-yl)-2-methoxybenzenesulfonamide

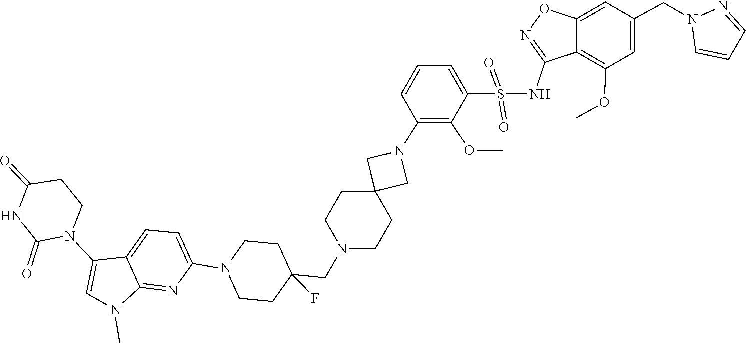 [SMILES: COc1c(N2CC3(CCN(CC4(F)CCN(c5ccc6c(N7CCC(=O)NC7=O)cn(C)c6n5)CC4)CC3)C2)cccc1S(=O)(=O)Nc1noc2cc(Cn3cccn3)cc(OC)c12]

Step 1. 1-(6-Chloro-1-methyl-1H-pyrrolo[2,3-b]pyridin-3-yl)dihydropyrimidine-2,4(1H,3H)-dione

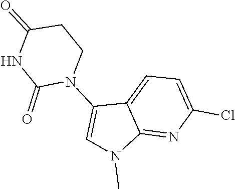 [SMILES: Cn1cc(N2CCC(=O)NC2=O)c2ccc(Cl)nc21]

The title compound was synthesized by procedures analogous to that described in Example 92, Steps 2-3, using 6-chloro-3-iodo-1-methyl-1H-pyrrolo[2,3-b]pyridine. LCMS calc. for C₁₂H₁₂ClN₄O₂[M+H]⁺: m/z=279.1; Found: 279.4.

### Step 2. 1-(6-(4-Fluoro-4-(hydroxymethyl)piperidin-1-yl)-1-methyl-1H-pyrrolo[2,3-b]pyridin-3-yl)dihydropyrimidine-2,4(1H,3H)-dione

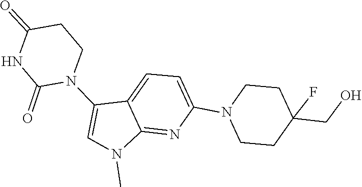 [SMILES: Cn1cc(N2CCC(=O)NC2=O)c2ccc(N3CCC(F)(CO)CC3)nc21]

A nitrogen sparged mixture of 1-(6-chloro-1-methyl-1H-pyrrolo[2,3-b]pyridin-3-yl)-dihydropyrimidine-2,4(1H,3H)-dione (2.70 g, 9.69 mmol), (4-fluoropiperidin-4-yl)methanol (1.29 g, 9.69 mmol), cesium carbonate (9.47 g, 29.1 mmol), and Pd-PEPPSI-iHeptCl (CAS: 1814936-54-33, 943 mg, 0.969 mmol) in 1,4-dioxane (40 mL) was stirred at 110° C. for 7 h. The mixture was concentrated and the residue was diluted with water and extracted with DCM. The combined organic layers were dried over anhydrous sodium sulfate, filtered, and concentrated. The crude material was purified by silica gel chromatography (0-6% MeOH/DCM) to afford the title compound (2.67 g, 7.10 mmol, 73.3%) as yellow brown solid. LCMS calc. for C₁₈H₂₃FN₅O₃[M+H]⁺: m/z=376.2; Found: 376.4.

### Step 3. 1-(3-(2,4-Dioxotetrahydropyrimidin-1(2H)-yl)-1-methyl-1H-pyrrolo[2,3-b]pyridin-6-yl)-4-fluoropiperidine-4-carbaldehyde

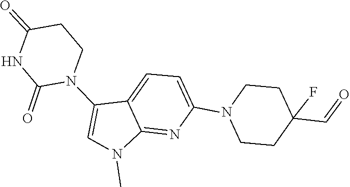 [SMILES: Cn1cc(N2CCC(=O)NC2=O)c2ccc(N3CCC(F)(C=O)CC3)nc21]

To a mixture of 1-(6-(4-fluoro-4-(hydroxymethyl)piperidin-1-yl)-1-methyl-1H-pyrrolo[2,3-b]pyridin-3-yl)dihydropyrimidine-2,4(1H,3H)-dione (1.500 g, 4.00 mmol) in DCM (12 mL) and DMSO (4 mL) was added triethylamine (2.43 g, 24.0 mmol). The content was sparged with nitrogen, cooled to 0° C., and sulfurtrioxide pyridine complex (3.18 g, 20.0 mmol) was added. The mixture was stirred at 0° C. for 30 min. The mixture was warmed to room temperature diluted with water and extracted with EtOAc. The combined organic layers were washed with brine, dried over MgSO₄, filtered, and concentrated. The crude material was purified by silica gel chromatography (0-80% EtOAc/DCM) to afford the title compound (590 mg, 1.58 mmol, 39.5%) as a white solid. LCMS calc. for C₁₈H₂₁FN₅O₃ [M+H]⁺: m/z=374.2; Found: 374.4.

### Step 4. N-(6-((1H-Pyrazol-1-yl)methyl)-4-methoxybenzo[d]isoxazol-3-yl)-3-(7-((1-(3-(2,4-dioxotetrahydropyrimidin-1(2H)-yl)-1-methyl-1H-pyrrolo[2,3-b]pyridin-6-yl)-4-fluoropiperidin-4-yl)methyl)-2, 7-diazaspiro[3.5]nonan-2-yl)-2-methoxybenzenesulfonamide

The title compound was synthesized by procedures analogous to that described in Example 2, using N-(6-((1H-pyrazol-1-yl)methyl)-4-methoxybenzo[d]isoxazol-3-yl)-2-methoxy-3-(2,7-diazaspiro[3.5]nonan-2-yl)benzenesulfonamide (which can be prepared as described in WO2023245150 A1). LCMS calc. for C₄₄H₅₁FN₁₁O₇S [M+H]⁺: m/z=896.4; Found: 896.4.

### Example 101. 3-[7-[[4-[4-(2,6-Dioxopiperidin-3-yl)phenyl]piperazin-1-yl]methyl]-2-azaspiro[3.5]nonan-2-yl]-5-fluoro-2-methoxy-N-[4-methoxy-6-(pyrazol-1-ylmethyl)-1,2-benzoxazol-3-yl]benzenesulfonamide

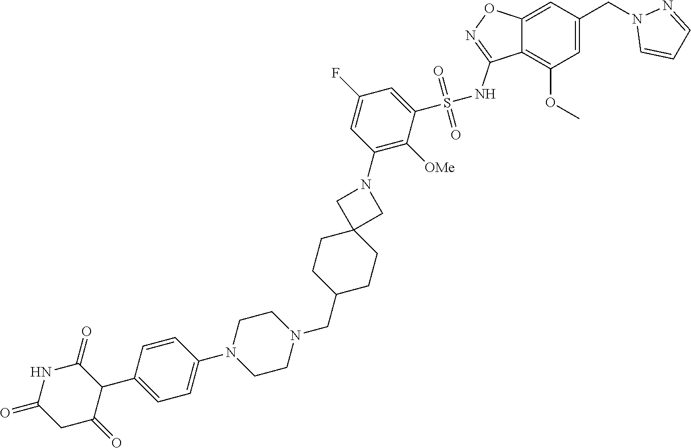 [SMILES: COc1c(N2CC3(CCC(CN4CCN(c5ccc(C6C(=O)CC(=O)NC6=O)cc5)CC4)CC3)C2)cc(F)cc1S(=O)(=O)Nc1noc2cc(Cn3cccn3)cc(OC)c12]

Step 1. 3-Bromo-5-fluoro-2-methoxybenzenesulfonyl chloride

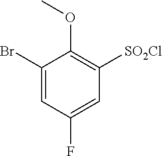

A solution of tert-butyl nitrite (1.33 g, 12.9 mmol) in MeCN (5 mL) was added dropwise to a mixture of 3-bromo-5-fluoro-2-methoxyaniline (2.36 g, 10.7 mmol), copper (II) chloride anhydrous (72.1 mg, 0.536 mmol), DABSO (1.55 g, 6.44 mmol), and hydrochloric acid (12N 1.79 mL, 21.45 mmol) in MeCN (25 mL). The mixture was stirred for 4 h. The reaction was quenched with cold water (100 mL) and extracted with EtOAc (75 mL×3). The combined organic phases were dried over Na₂SO₄, filtered, and concentrated. The crude material was purified by silica gel chromatography (0-10% MTBE/heptane) to afford the title compound (1.76 g, 5.81 mmol, 54.2%), as an amber oil. An aliquot was quenched with morpholine for LCMS analysis. LCMS calc. for C₁₁H₁₄BrFNO₄S [M+H]⁺: m/z=354.0; Found: 354.2.

### Step 2. N-(6-((1H-Pyrazol-1-yl)methyl)-4-methoxybenzo[d]isoxazol-3-yl)-3-bromo-5-fluoro-2-methoxybenzenesulfonamide

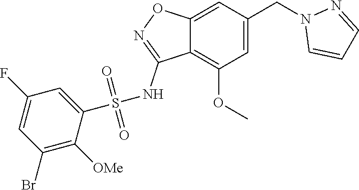 [SMILES: COc1c(Br)cc(F)cc1S(=O)(=O)Nc1noc2cc(Cn3cccn3)cc(OC)c12]

The title compound was synthesized by procedures analogous to that described in Example 12, Step 1. LCMS calc. for C₁₉H₁₇BrFN₄O₅S [M+H]⁺: m/z=511.0, 513.0; Found: 511.2, 513.1.

### Step 3. N-(6-((1H-Pyrazol-1-yl)methyl)-4-methoxybenzo[d]isoxazol-3-yl)-3-(7-((4-(4-(2,6-dioxopiperidin-3-yl)phenyl)piperazin-1-yl)methyl)-2-azaspiro[3.5]nonan-2-yl)-5-fluoro-2-methoxybenzenesulfonamide

The title compound was synthesized by procedures analogous to that described in Example 35, Steps 1-5, using (2-azaspiro[3.5]nonan-7-yl)methanol and 3-(4-(piperazin-1-yl)phenyl)piperidine-2,6-dione. LCMS calc. for C₄₃H₅₀FN₈O₇S [M+H]⁺: m/z=841.3; Found 841.8.

### Example 102. N-(6-((1H-Pyrazol-1-yl)methyl)-4-methoxybenzo[d]isoxazol-3-yl)-3-((1S,6S)-3-((1-(3-(2,4-dioxotetrahydropyrimidin-1(2H)-yl)-1-methyl-1H-indazol-6-yl)piperidin-4-yl)methyl)-3,7-diazabicyclo[4.2.0]octan-7-yl)-2-methoxy-6-methylbenzenesulfonamide

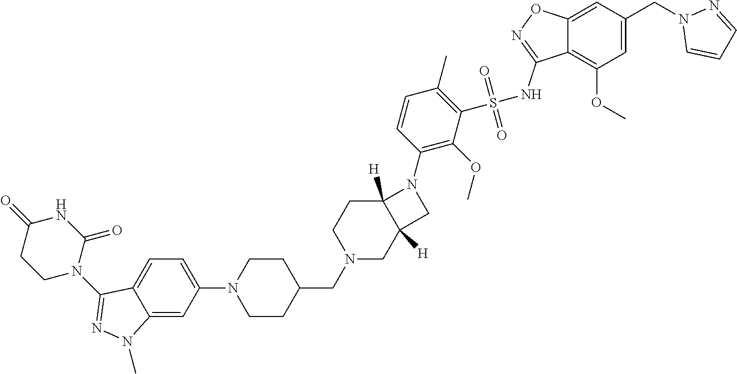 [SMILES: COc1c(N2C[C@@H]3CN(CC4CCN(c5ccc6c(N7CCC(=O)NC7=O)nn(C)c6c5)CC4)CC[C@@H]32)ccc(C)c1S(=O)(=O)Nc1noc2cc(Cn3cccn3)cc(OC)c12]

### Step 1. 1-Bromo-2-methoxy-4-methyl-3-nitrobenzene

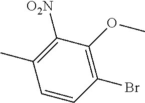

To a solution of 6-bromo-3-methyl-2-nitrophenol (930 mg, 4.01 mmol) and potassium carbonate (1.66 g, 12.0 mmol) in DMF (10 mL) was added iodomethane (1.71 g, 12.0 mmol). The mixture was stirred overnight. The mixture was poured in water and extracted with ethyl acetate. The organic layers were dried over anhydrous sodium sulfate, filtered, and concentrated. The crude material was purified by silica gel chromatography (0-20% EtOAc/heptane) to afford the title compound (1.01 g, 4.10 mmol, quantitative yield) as colorless oil. ¹H NMR (300 MHz, CDCl₃) δ 7.53 (d, J=8.3 Hz, 1H), 6.93 (d, J=8.3 Hz, 1H), 3.95 (s, 3H), 2.27 (s, 3H).

### Step 2. 3-Bromo-2-methoxy-6-methylaniline

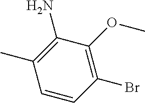

To a solution of 1-bromo-2-methoxy-4-methyl-3-nitrobenzene (836 mg, 3.40 mmol) in methanol (10 mL) and water (2.5 mL) was added ammonium chloride (1.09 g, 20.4 mmol). The mixture was heated to 80° C. and iron powder (1.90 g, 34.0 mmol) was added. The mixture was heated at 80° C. for 4 h. The material was centrifuged and the residue was washed several times with methanol. The organic pool was adsorbed on silica gel and purified by silica column chromatography (0-50% EtOAc/hexanes) to afford the title compound (739 mg, 3.42 mmol, quantitative) as a light yellow oil. ¹H NMR (300 MHz, CDCl₃) δ 6.83 (d, J=8.1 Hz, 1H), 6.70 (d, J=8.1 Hz, 1H), 3.87 (s, 2H), 3.83 (s, 3H), 2.13 (s, 3H).

### Step 3. 1-Bromo-3-iodo-2-methoxy-4-methylbenzene

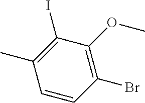

A solution of 3-bromo-2-methoxy-6-methylaniline (655 mg, 3.03 mmol) in water (7 mL) was cooled in an ice-bath and sulfuric acid (0.969 mL, 18.2 mmol) was added. Sodium nitrite solution (3.13 g, 4.55 mmol) in water (1 mL) was added dropwise. The reaction mixture was stirred at 0° C. for 1 h. A solution of potassium iodide (1.51 g, 9.09 mmol) in water (1 mL) was added dropwise. The reaction was stirred at room temperature for 10 min, then 100° C. for 7 h. The reaction was quenched with water and extracted with ethyl acetate. The organic pool was dried over anhydrous sodium sulfate, filtered, and concentrated. The crude material was purified by silica gel chromatography (0-5% EtOAc/heptane) to afford the title compound (873 mg, 2.67 mmol, 88.1%) as an oil. ¹H NMR (300 MHz, CDCl₃) δ 7.40 (d, J=8.1 Hz, 1H), 6.89 (d, J=8.1 Hz, 1H), 3.87 (s, 3H), 2.44 (s, 3H).

### Step 4. 3-Bromo-2-methoxy-6-methylbenzenesulfonyl chloride

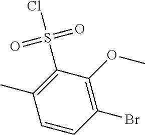

The title compound was synthesized by procedures analogous to that described in Example 21, Steps 2-3. ¹H NMR (300 MHz, CDCl₃) δ 7.74 (d, J=8.3 Hz, 1H), 7.00 (dd, J=8.3, 0.9 Hz, 1H), 4.09 (s, 3H), 2.68 (s, 3H).

### Step 5. 3-Bromo-2-methoxy-N-[4-methoxy-6-(pyrazol-1-ylmethyl)-1,2-benzoxazol-3-yl]-6-methylbenzenesulfonamide

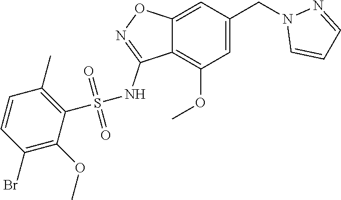 [SMILES: COc1c(Br)ccc(C)c1S(=O)(=O)Nc1noc2cc(Cn3cccn3)cc(OC)c12]

The title compound was synthesized by procedures analogous to that described in Example 12, Step 1. LCMS calc. for C₂₀H₂₀BrN₄O₅S [M+H]⁺: m/z=507.0, 509.0; Found 507.2, 509.2.

### Step 6. N-(6-((1H-Pyrazol-1-yl)methyl)-4-methoxybenzo[d]isoxazol-3-yl)-3-((1S,6S)-3-((1-(3-(2,4-dioxotetrahydropyrimidin-1(2H)-yl)-1-methyl-1H-indazol-6-yl)piperidin-4-yl)methyl)-3, 7-diazabicyclo[4.2.0]octan-7-yl)-2-methoxy-6-methylbenzenesulfonamide

The title compound was synthesized by procedures analogous to that described in Example 47, Steps 1-2. LCMS calc. for C₄₄H₅₂N₁₁O₇S [M+H]⁺: m/z=878.4; Found: 878.7.

### Example 103. N-(6-((1H-Pyrazol-1-yl)methyl)-4-methoxybenzo[d]isoxazol-3-yl)-3-(7-((1-(3-(2,6-dioxopiperidin-3-yl)-5-fluoro-1-methyl-1H-indazol-6-yl)piperidin-4-yl)methyl)-2,7-diazaspiro[3.5]nonan-2-yl)-5-fluoro-2,6-dimethoxybenzenesulfonamide

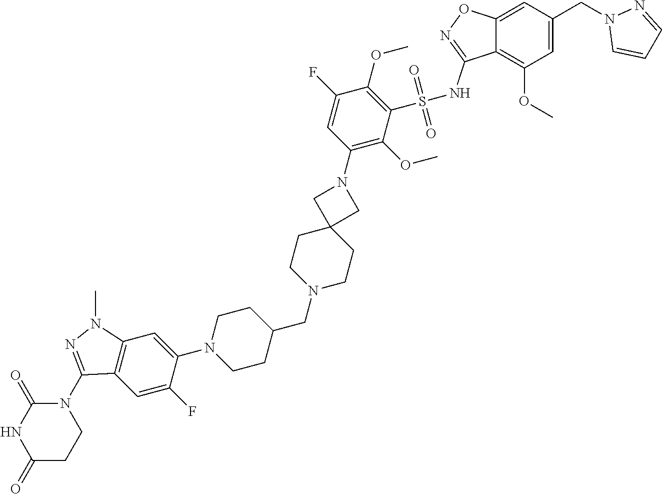 [SMILES: COc1c(F)cc(N2CC3(CCN(CC4CCN(c5cc6c(cc5F)c(N5CCC(=O)NC5=O)nn6C)CC4)CC3)C2)c(OC)c1S(=O)(=O)Nc1noc2cc(Cn3cccn3)cc(OC)c12]

The title compound was synthesized by procedures analogous to that described in Example 2, using 1-(3-(2,6-dioxopiperidin-3-yl)-5-fluoro-1-methyl-1H-indazol-6-yl)piperidine-4-carbaldehyde (which can be prepared as described in WO2025131111 A1) and N-(6-((1H-pyrazol-1-yl)methyl)-4-methoxybenzo[d]isoxazol-3-yl)-3-fluoro-2,6-dimethoxy-5-(2,7-diazaspiro[3.5]nonan-2-yl)benzenesulfonamide (from Example 58, Step 4). LCMS calc. for C₄₆H₅₃F₂N₁₀O₈S [M+H]: m/z=943.4; Found: 943.7.

### Example 104. N-(6-((1H-Pyrazol-1-yl)methyl)-4-methoxybenzo[d]isoxazol-3-yl)-3-((3R)-3-((4-(3-(2,6-dioxopiperidin-3-yl)-1-methyl-1H-indazol-6-yl)piperidin-1-yl)methyl)-piperidin-1-yl)-2,6-dimethoxybenzenesulfonamide

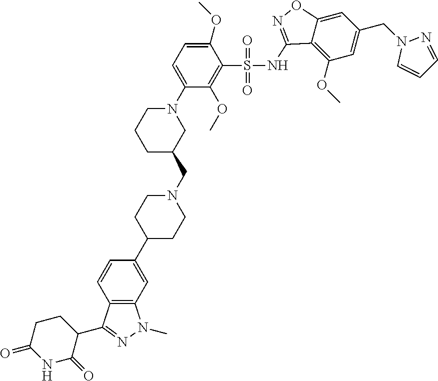 [SMILES: COc1ccc(N2CCC[C@H](CN3CCC(c4ccc5c(C6CCC(=O)NC6=O)nn(C)c5c4)CC3)C2)c(OC)c1S(=O)(=O)Nc1noc2cc(Cn3cccn3)cc(OC)c12]

### Step 1. (s)-3-(((tert-Butyldimethylsilyl)oxy)methyl)piperidine

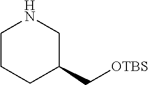 [SMILES: CC(C)(C)[Si](C)(C)OC[C@H]1CCCNC1]

The title compound was synthesized by procedures analogous to that described in Example 35, Step 1, using (S)-piperidin-3-ylmethanol. LCMS calc. for C₁₂H₂₈NOSi [M+H]⁺: m/z=230.2; Found: 230.3.

### Step 2. N-(6-((1H-Pyrazol-1-yl)methyl)-4-methoxybenzo[d]isoxazol-3-yl)-3-((3R)-3-((4-(3-(2,6-dioxopiperidin-3-yl)-1-methyl-1H-indazol-6-yl)piperidin-1-yl)methyl)piperidin-1-yl)-2,6-dimethoxybenzenesulfonamide

The title compound was synthesized by procedures analogous to that described in Example 35, Steps 3-5, using 3-(1-methyl-6-(piperidin-4-yl)-1H-indazol-3-yl)piperidine-2,6-dione and N-(6-((1H-pyrazol-1-yl)methyl)-4-methoxybenzo[d]isoxazol-3-yl)-3-bromo-2,6-dimethoxybenzenesulfonamide (which can be prepared as described in WO2024039901 A₂). LCMS calc. for C₄₄H₅₂N₉O₈S [M+H]⁺: m/z=866.4; Found: 866.8.

### Example 105. 3-[6-[[4-[3-(2,6-Dioxopiperidin-3-yl)-1-methylindazol-6-yl]piperidin-1-yl]methyl]-2-pyridinyl]-2,6-dimethoxy-N-[4-methoxy-6-(pyrazol-1-ylmethyl)-1,2-benzoxazol-3-yl]benzenesulfonamide

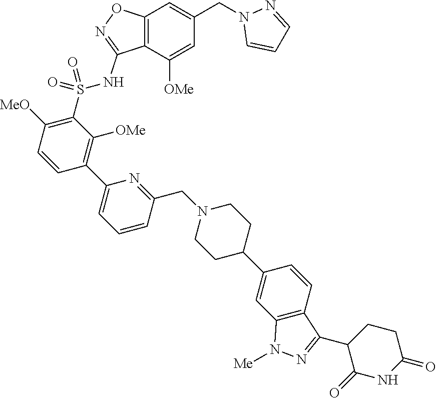 [SMILES: COc1ccc(-c2cccc(CN3CCC(c4ccc5c(C6CCC(=O)NC6=O)nn(C)c5c4)CC3)n2)c(OC)c1S(=O)(=O)Nc1noc2cc(Cn3cccn3)cc(OC)c12]

### Step 1. 3-Bromo-2,6-dimethoxybenzenesulfonylfluoride

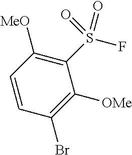 [SMILES: COc1ccc(Br)c(OC)c1S(=O)(=O)F]

A solution of 3-bromo-2,6-dimethoxybenzenesulfonyl chloride (921 mg, 2.92 mmol) and potassium bifluoride (570 mg, 7.30 mmol) in MeCN (8 mL) and water (4 mL) was stirred for 18 h. The reaction was diluted with EtOAc (100 mL), washed with brine (50 mL×3), dried over Na₂SO₄, filtered, and concentrated. The residue was purified by silica gel chromatography (0-25% EtOAc/heptane) to afford the title compound (527 mg, 1.76 mmol, 60.3%) as a white solid. ¹H NMR (300 MHz, CDCl₃) δ 7.79 (d, J=9.1 Hz, 1H), 6.78 (d, J=9.1 Hz, 1H), 4.00 (s, 3H), 3.97 (s, 3H).

### Step 2. 2,6-Dimethoxy-3-(4,4,5,5-tetramethyl-1, 3,2-dioxaborolan-2-yl)benzene-sulfonylfluoride

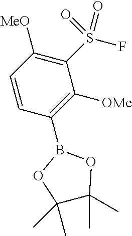 [SMILES: COc1ccc(B2OC(C)(C)C(C)(C)O2)c(OC)c1S(=O)(=O)F]

A mixture of 3-bromo-2,6-dimethoxybenzenesulfonyl fluoride (2.19 g, 7.31 mmol), bis(pinacolato)diboron (2.78 g, 11.0 mmol), [1,1′-bis(diphenylphosphino)ferrocene]-dichloro-palladium(II) (535 mg, 0.731 mmol), and KOAc (2.02 g, 21.9 mmol) in 1,4-dioxane (25 mL) was heated at 100° C. for 18 h under a nitrogen atmosphere. The reaction was cooled to ambient temperature, diluted with EtOAc (150 mL), washed with water (75 mL) and saturated NaHCO₃ (aq.) solution (75 mL), dried over Na₂SO₄, filtered, and concentrated. The residue was purified by silica gel chromatography (0-25% MTBE/heptane) to afford the title compound (1.46 g, 4.22 mmol, 57.7%) as white solid. ¹H NMR (300 MHz, CDCl₃) δ 7.99 (d, J=8.6 Hz, 1H), 6.81 (d, J=8.6 Hz, 1H), 3.97 (s, 6H), 1.35 (s, 12H).

### Step 3. 2-Bromo-6-(dimethoxymethyl)pyridine

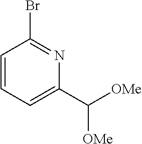 [SMILES: COC(OC)c1cccc(Br)n1]

To a solution of 6-bromo-2-pyridinecarboxaldehyde (1.86 g, 9.99 mmol) and trimethylorthoformate (2.74 g, 25.8 mmol) in MeOH (20 mL) was added p-TSOH H₂O (0.393 g, 2.07 mmol) and the mixture was heated at 70° C. for 24 h. The reaction was cooled to ambient temperature, diluted with EtOAc (50 mL), washed with sat. NaHCO₃ (aq.) solution (25 mL), water (25 mL), dried over Na₂SO₄, filtered, and concentrated. The crude material was purified by silica gel chromatography (0-40% EtOAc/heptane) to afford the title compound (2.24 g, 9.64 mmol, 96.5%), as colorless oil. ¹H NMR (300 MHz, CDCl₃) δ 7.65-7.40 (m, 3H), 5.30 (s, 1H), 3.41 (s, 6H).

### Step 4. 3-[6-(Dimethoxymethyl)-2-pyridinyl]-2,6-dimethoxybenzenesulfonyl fluoride

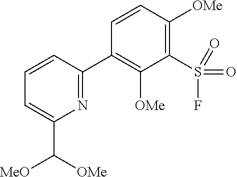 [SMILES: COc1ccc(-c2cccc(C(OC)OC)n2)c(OC)c1S(=O)(=O)F]

A mixture of 2,6-dimethoxy-3-(4,4,5,5-tetramethyl-1,3,2-dioxaborolan-2-yl)benzenesulfonyl fluoride (287 mg, 0.830 mmol), 2-bromo-6-(dimethoxymethyl)pyridine (160 mg, 0.690 mmol), bis(diphenylphosphino)ferrocene]dichloropalladium(II) (25 mg, 0.035 mmol) and Cs₂CO₃ (675 mg, 2.07 mmol) in water (0.20 mL) and 1,4-dioxane (1.8 mL) was heated at 100° C. under a nitrogen atmosphere for 18 h. The reaction was cooled to ambient temperature and diluted with EtOAc (50 mL), washed with water (30 mL) and brine (30 mL), dried over Na₂SO₄, filtered, and concentrated. The crude material was purified by silica gel chromatography (0-75% EtOAc/heptane) to afford the title compound (210 mg, 0.57 mmol, 83%) as a yellow solid. ¹H NMR (300 MHz, CDCl₃) δ 8.09 (d, J=8.9 Hz, 1H), 7.97-7.74 (m, 2H), 7.55 (dd, J=6.3, 2.4 Hz, 1H), 6.96 (d, J=9.0 Hz, 1H), 5.41 (s, 1H), 4.01 (s, 3H), 3.59 (s, 3H), 3.45 (s, 6H). LCMS calc. for C₁₆H₁₉FNO₆S [M+H]⁺: m/z=372.1; Found: 372.2.

### Step 5. 3-[6-(Dimethoxymethyl)-2-pyridinyl]-2,6-dimethoxy-N-[4-methoxy-6-(pyrazol-1-ylmethyl)-1,2-benzoxazol-3-yl]benzenesulfonamide

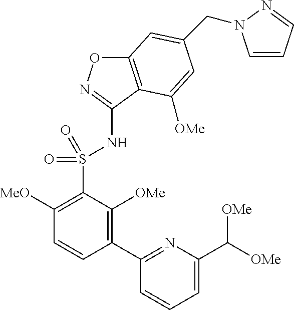 [SMILES: COc1ccc(-c2cccc(C(OC)OC)n2)c(OC)c1S(=O)(=O)Nc1noc2cc(Cn3cccn3)cc(OC)c12]

KO^{t}Bu (1.0 M in THF, 0.86 mmol, 0.86 mL) was added dropwise to a solution of 3-[6-(dimethoxymethyl)-2-pyridinyl]-2,6-dimethoxybenzenesulfonyl fluoride (201 mg, 0.540 mmol) and 4-methoxy-6-(1H-pyrazol-1-ylmethyl)-1,2-benzoxazol-3-amine (52.8 mg, 0.216 mmol) (which can be prepared as described in WO2023245150 A1) in THF (1 mL) at 0° C. The mixture was stirred for 30 min, quenched with 10% citric acid (aq.) solution (10 mL), and extracted with EtOAc (10 mL×3). The combined organic phases were dried over Na₂SO₄, filtered, and concentrated. The crude material was purified by prep-HPLC on a C18 column (20.0-70.0% CH₃CN/0.05% TFA (aq.)) to afford the title compound (64 mg, 0.11 mmol, 50%) as white solid. LCMS calculated for C₂₈H₃₀N₅O₈S [M+H]⁺: m/z=596.2; Found: 596.3.

### Step 6. 3-(6-Formyl-2-pyridinyl)-2,6-dimethoxy-N-[4-methoxy-6-(pyrazol-1-ylmethyl)-]1,2-benzoxazol-3-yl]benzenesulfonamide

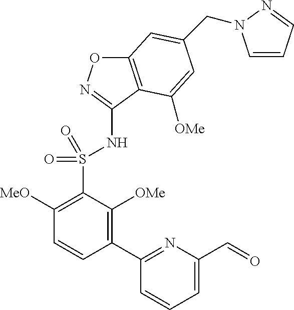 [SMILES: COc1ccc(-c2cccc(C=O)n2)c(OC)c1S(=O)(=O)Nc1noc2cc(Cn3cccn3)cc(OC)c12]

A solution of HCl (4M aq., 0.50 mL) was added to a solution of 3-[6-(dimethoxy-methyl)-2-pyridinyl]-2,6-dimethoxy-N-[4-methoxy-6-(pyrazol-1-ylmethyl)-1,2-benzoxazol-3-yl]benzenesulfonamide (45.6, 0.0766 mmol) in THF (0.50 mL) and the reaction was heated at 60° C. for 3 h. Volatiles were evaporated and the residue was adjusted to pH~8 with saturated NaHCO₃ (aq.) solution. The mixture was extracted with DCM (5 mL×3). The combined organic phases were dried over Na₂SO₄, filtered, and concentrated to afford the title compound (45.5 mg, 0.0828 mmol, 95.2%) as off-white solid. LCMS calc. for C₂₆H₂₄N₅O₇S [M+H]⁺: m/z=550.1; Found: 550.2.

### Step 7. 3-[6-[[4-[3-(2,6-Dioxopiperidin-3-yl)-1-methylindazol-6-yl]piperidin-1-yl]methyl]-2-pyridinyl]-2,6-dimethoxy-N-[4-methoxy-6-(pyrazol-1-ylmethyl)-1,2-benzoxazol-3-yl]benzenesulfonamide

The title compound was synthesized by procedures analogous to that described in Example 35, Step 5, using 3-(1-methyl-6-(piperidin-4-yl)-1H-indazol-3-yl)piperidine-2,6-dione. ¹H NMR (300 MHz, CD₃OD) δ 8.02-7.89 (m, 2H), 7.87-7.74 (m, 2H), 7.69 (d, J=8.4 Hz, 1H), 7.61-7.44 (m, 2H), 7.41 (s, 1H), 7.09 (dd, J=18.9, 8.7 Hz, 2H), 6.70 (d, J=4.8 Hz, 2H), 6.36 (s, 1H), 5.45 (s, 2H), 4.62 (s, 2H), 4.36 (dd, J=9.1, 5.1 Hz, 1H), 4.00 (d, J=6.0 Hz, 6H), 3.88 (s, 3H), 3.83-3.68 (m, 2H), 3.59 (s, 3H), 3.46-3.33 (m, 2H), 3.21-3.00 (m, 1H), 2.84-2.70 (m, 2H), 2.53-2.13 (m, 6H). LCMS calc. for C₄₄H₄₆N₉O₈S [M+H]⁺: m/z=860.3; Found: 860.6.

### Example 106. N-(6-((1H-Pyrazol-1-yl)methyl)-4-methoxybenzo[d]isoxazol-3-yl)-3-(4-((4-(3-(2,6-dioxopiperidin-3-yl)-1-methyl-1H-indazol-6-yl)piperidin-1-yl)methyl)pyridin-2-yl)-2,6-dimethoxybenzenesulfonamide

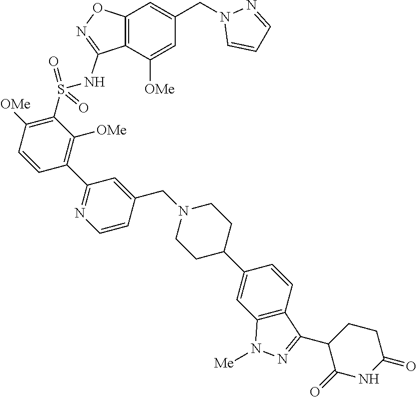 [SMILES: COc1ccc(-c2cc(CN3CCC(c4ccc5c(C6CCC(=O)NC6=O)nn(C)c5c4)CC3)ccn2)c(OC)c1S(=O)(=O)Nc1noc2cc(Cn3cccn3)cc(OC)c12]

The title compound was synthesized by procedures analogous to that described in Example 105, Steps 1-7, using 2-bromoisonicotinaldehyde. ¹H NMR (300 MHz, CD₃OD) δ 8.82 (d, J=5.1 Hz, 1H), 8.08 (s, 1H), 7.90 (d, J=8.9 Hz, 1H), 7.78 (d, J=2.0 Hz, 1H), 7.70 (d, J=8.4 Hz, 1H), 7.64 (d, J=5.2 Hz, 1H), 7.56 (d, J=1.3 Hz, 1H), 7.38 (s, 1H), 7.09 (d, J=8.9 Hz, 2H), 6.74 (d, J=17.6 Hz, 2H), 6.37 (t, J=2.1 Hz, 1H), 5.47 (s, 2H), 4.55 (s, 2H), 4.36 (dd, J=9.2, 5.2 Hz, 1H), 4.01 (s, 6H), 3.89 (s, 3H), 3.72-3.52 (m, 5H), 3.30-3.21 (m, 2H), 3.17-3.04 (m, 1H), 2.84-2.68 (m, 2H), 2.56-2.27 (m, 2H), 2.26-2.05 (m, 4H). LCMS calc. for C₄₄H₄₆N₉O₈S [M+H]⁺: m/z=860.3; Found: 860.6.

### Example 107. N-(6-((1H-Pyrazol-1-yl)methyl)-4-methoxybenzo[d]isoxazol-3-yl)-3-(8-((1-(3-(2,4-dioxotetrahydropyrimidin-1(2H)-yl)-1-methyl-1H-indazol-6-yl)piperidin-4-yl)methyl)-8-azabicyclo[3.2.1]octan-3-yl)-5-fluoro-2,6-dimethoxybenzenesulfonamide

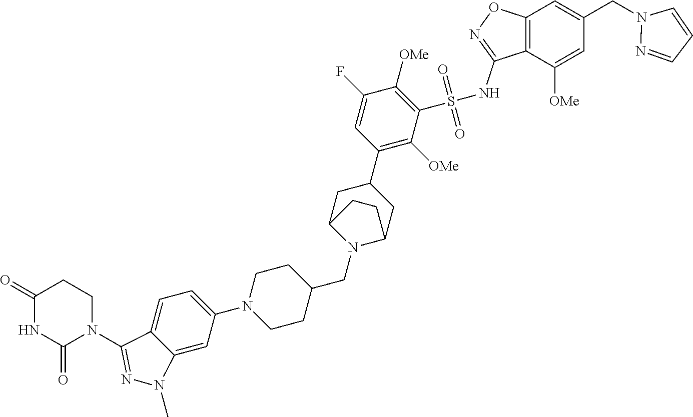 [SMILES: COc1c(F)cc(C2CC3CCC(C2)N3CC2CCN(c3ccc4c(N5CCC(=O)NC5=O)nn(C)c4c3)CC2)c(OC)c1S(=O)(=O)Nc1noc2cc(Cn3cccn3)cc(OC)c12]

### Step 1. tert-Butyl 3-(5-fluoro-3-fluorosulfonyl-2,4-dimethoxyphenyl)-8-azabicyclo[3.2.1]octane-8-carboxylate

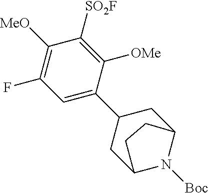 [SMILES: COc1c(F)cc(C2CC3CCC(C2)N3C(=O)OC(C)(C)C)c(OC)c1S(=O)(=O)F]

A mixture of pyridin-2-carboximidamidhydrochloride (7.5 mg, 0.047 mmol), 3-bromo-5-fluoro-2,6-dimethoxybenzenesulfonyl fluoride (75.1 mg, 0.237 mmol), Mn powder (39.0 mg, 0.711 mmol), tert-butyl 3-iodo-8-azabicyclo[3.2.1]octane-8-carboxylate (120 mg, 0.355 mmol), and tetrabutylammonium iodide (17.5 mg, 0.0474 mmol) in DMA (2 mL) was heated at 55° C. under a nitrogen atmosphere for 3 h. The reaction was diluted with EtOAc (50 mL), washed with water (25 mL) and brine (25 mL), dried over Na₂SO₄, filtered, and concentrated. The residue was purified by silica gel chromatography (0-50% EtOAc/heptane) to afford the title compound (125 mg, 0.281 mmol, quantitative) as white solid. LCMS calc. for C₂₀H₂₈F₂NO₆S [M+H]⁺: m/z=448.2; Found: 448.4.

### Step 2. tert-Butyl 3-[5-fluoro-2,4-dimethoxy-3-[[4-methoxy-6-(pyrazol-1-ylmethyl)-1,2-benzoxazol-3-yl]sulfamoyl]phenyl]-8-azabicyclo[3.2.1]octane-8-carboxylate

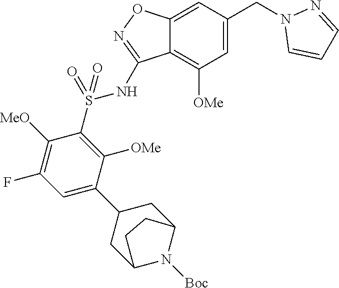 [SMILES: COc1c(F)cc(C2CC3CCC(C2)N3C(=O)OC(C)(C)C)c(OC)c1S(=O)(=O)Nc1noc2cc(Cn3cccn3)cc(OC)c12]

The title compound was synthesized by procedures analogous to that described in Example 12, Step 1. LCMS calc. for C₃₂H₃₉FN₅O₈S [M+H]⁺: m/z=672.2; Found: 672.6.

### Step 3. 3-(8-Azabicyclo[3.2.1]octan-3-yl)-5-fluoro-2,6-dimethoxy-N-[4-methoxy-6-(pyrazol-1-ylmethyl)-1,2-benzoxazol-3-yl]benzenesulfonamide

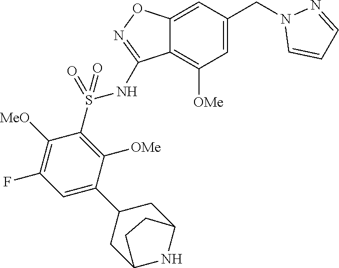 [SMILES: COc1c(F)cc(C2CC3CCC(C2)N3)c(OC)c1S(=O)(=O)Nc1noc2cc(Cn3cccn3)cc(OC)c12]

To a solution of tert-butyl 3-[5-fluoro-2,4-dimethoxy-3-[[4-methoxy-6-(pyrazol-1-ylmethyl)-1,2-benzoxazol-3-yl]sulfamoyl]phenyl]-8-azabicyclo[3.2.1]octane-8-carboxylate (21.8 mg, 0.0325 mmol) in DCM (1.2 mL) was added TFA (0.40 mL) at 0° C. The reaction mixture was stirred at 0° C. for 1 h. The volatiles were evaporated and the crude material was purified by prep-HPLC on a C18 column (20.0-50.0% CH₃CN/0.05% TFA (aq.)) to afford the title compound as a TFA salt (5.8 mg, 0.0085 mmol, 26%), a white solid. LCMS calc. for C₂₇H₃₁FN₅O₆S [M+H]⁺: m/z=572.2; Found: 572.5.

### Step 4. 3-[8-[[1-[3-(2,4-Dioxo-]1,3-diazinan-1-yl)-1-methylindazol-6-yl]piperidin-4-yl]methyl]-8-azabicyclo[3.2.1]octan-3-yl]-5-fluoro-2,6-dimethoxy-N-[4-methoxy-6-(pyrazol-1-ylmethyl)-1,2-benzoxazol-3-yl]benzenesulfonamide

The title compound was synthesized by procedures analogous to that described in Example 2, using 1-(3-(2,4-dioxotetrahydropyrimidin-1(2H)-yl)-1-methyl-1H-indazol-6-yl)piperidine-4-carbaldehyde (which can be prepared as described in WO2023249970 A1). LCMS calc. for C₄₅H₅₂FN₁₀O₈S [M+H]⁺: m/z=911.4; Found: 911.4.

### Example 108. N-(6-((1H-Pyrazol-1-yl)methyl)-4-methoxybenzo[d]isoxazol-3-yl)-3-(7-((1-(2-(2,6-dioxopiperidin-3-yl)-1-methyl-1H-pyrrolo[2,3-b]pyridin-6-yl)piperidin-4-yl)methyl)-2,7-diazaspiro[3.5]nonan-2-yl)-2-methoxybenzenesulfonamide

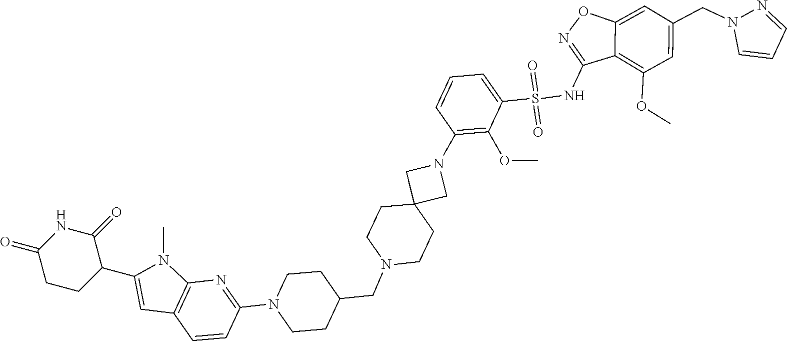 [SMILES: COc1c(N2CC3(CCN(CC4CCN(c5ccc6cc(C7CCC(=O)NC7=O)n(C)c6n5)CC4)CC3)C2)cccc1S(=O)(=O)Nc1noc2cc(Cn3cccn3)cc(OC)c12]

### Step 1. 6-Chloro-3-iodo-1-methyl-1H-pyrrolo[2,3-b]pyridine

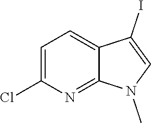

A mixture of 6-chloro-3-iodo-1H-pyrrolo[2,3-b]pyridine (327 mmol) in DMF (600 mL) was cooled to 0° C. Iodomethane (69 g, 490 mmol) was added dropwise and stirred at 15° C. for 1 hr. The reaction mixture was poured into ice water (1.5 L) and the mixture was extracted with ethyl acetate (600 mL×2). The combined organic layers were washed with Na₂S₂O₃ (300 mL×2) and brine, dried over Na₂SO₄, and filtered to afford the title compound (90 g, 308 mmol, 94%) as a yellow solid. LCMS calc. for C₈H₇ClIN₂ [M+H]⁺: m/z=292.9; Found: 292.9.

### Step 2. 2-(2,6-Bis(benzyloxy)pyridin-3-yl)-6-chloro-1-methyl-1H-pyrrolo[2,3-b]pyridine

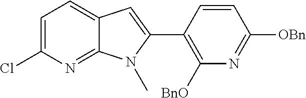 [SMILES: Cn1c(-c2ccc(OCc3ccccc3)nc2OCc2ccccc2)cc2ccc(Cl)nc21]

To a solution of 6-chloro-3-iodo-1-methyl-1H-pyrrolo[2,3-b]pyridine (40 g, 140 mmol) in dioxane (400 mL) and water (60 mL) was added 2,6-bis(benzyloxy)-3-(4,4,5,5-tetramethyl-1,3,2-dioxaborolan-2-yl)pyridine (57 g, 140 mmol) and cesium carbonate (133 g, 410 mmol). The mixture was sparged with nitrogen and [1,1′-bis(diphenylphosphino)-ferrocene]dichloropalladium(II) (5.5 g, 6.8 mmol) was added. The mixture was stirred at 70° C. for 16 h. The reaction mixture was cooled to room temperature, poured into water (400 mL) and extracted with ethyl acetate (400 mL×2). The combined organic layers were washed with brine, dried with Na₂SO₄, filtered, and concentrated. The crude material was purified by silica gel chromatography (2.5% EtOAc/petroleum ether) to afford the title compound (15 g, 33 mmol, 21%) as a white solid. LCMS calc. for C₂₇H₂₃ClN₃O₂[M+H]⁺: m/z=456.1; Found: 456.3.

### Step 3. 2-(2,6-bis(Benzyloxy)pyridin-3-yl)-6-(4-(dimethoxymethyl)piperidin-1-yl)-1-methyl-1H-pyrrolo[2,3-b]pyridine

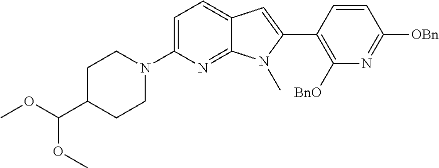 [SMILES: COC(OC)C1CCN(c2ccc3cc(-c4ccc(OCc5ccccc5)nc4OCc4ccccc4)n(C)c3n2)CC1]

To a mixture of 2-(2,6-bis(benzyloxy)pyridin-3-yl)-6-chloro-1-methyl-1H-pyrrolo-[2,3-b]pyridine (15 g, 33 mmol) in dioxane (150 mL) was added 4-(dimethoxymethyl)-piperidine (6.8 g, 43 mmol) and cesium carbonate (21.2 g, 65.8 mmol). The mixture was sparged with nitrogen and Pd₂(dba)₃ (3.0 g, 3.3 mmol) and Ruphos (CAS: 787618-22-8, 2.7 g, 6.6 mmol) were added. The mixture was stirred at 100° C. for 16 hrs. The reaction mixture was cooled to room temperature, poured into water (200 mL) and extracted with ethyl acetate (200 mL×2). The combined organic layers were washed with brine, dried with Na₂SO₄, filtered, and concentrated. The crude material was purified by silica gel chromatography (6.25% EtOAc/petroleum ether) to afford the title compound (14 g, 24 mmol, 56%) as a white solid. LCMS calc. for C₃₅H₃₉N₄O₄ [M+H]⁺: m/z=579.3; Found: 579.3.

### Step 4. 3-(6-(4-(Dimethoxymethyl)piperidin-1-yl)-1-methyl-1H-pyrrolo[2,3-b]pyridin-2-yl)piperidine-2,6-dione

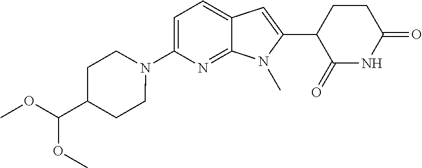 [SMILES: COC(OC)C1CCN(c2ccc3cc(C4CCC(=O)NC4=O)n(C)c3n2)CC1]

To a solution of 2-(2,6-bis(benzyloxy)pyridin-3-yl)-6-(4-(dimethoxymethyl)-piperidin-1-yl)-1-methyl-1H-pyrrolo[2,3-b]pyridine (14 g, 24 mmol) in THF/EtOH (140 mL/140 mL) was added Pd/C (1.4 g). The suspension was degassed under vacuum and purged with hydrogen (15 psi) several times. The mixture was stirred under a hydrogen atmosphere at 70° C. for 16 h. The reaction mixture was cooled to room temperature and filtered through a pad of silica gel, washing with THF. The combined filtrate was concentrated, and the crude product was purified by triturating with ethyl acetate (20 mL) at room temperature for 1 h. After filtration, the filter cake was washed with ethyl acetate (10 mL) and concentrated to afford the title compound (6.0 g, 15 mmol, 63%) as a yellow solid. LCMS calc. for C₂₁H₂₉N₄O₄ [M+H]⁺: m/z=401.2; Found: 401.1.

### Step 5. 1-(2-(2,6-Dioxopiperidin-3-yl)-1-methyl-1H-pyrrolo[2,3-b]pyridin-6-yl)piperidine-4-carbaldehyde

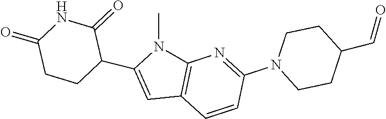 [SMILES: Cn1c(C2CCC(=O)NC2=O)cc2ccc(N3CCC(C=O)CC3)nc21]

The title compound was synthesized by procedures analogous to that described in Example 4, Step 4. LCMS calc. for C₁₉H₂₃N₄O₃ [M+H]⁺: m/z=355.2; found: 355.1.

### Step 6. N-(6-((1H-Pyrazol-1-yl)methyl)-4-methoxybenzo[d]isoxazol-3-yl)-3-(7-((1-(2-(2,6-dioxopiperidin-3-yl)-1-methyl-1H-pyrrolo[2,3-b]pyridin-6-yl)piperidin-4-yl)methyl)-2, 7-diazaspiro[3.5]nonan-2-yl)-2-methoxybenzenesulfonamide

The title compound was synthesized by procedures analogous to that described in Example 2, using N-(6-((1H-pyrazol-1-yl)methyl)-4-methoxybenzo[d]isoxazol-3-yl)-2-methoxy-3-(2,7-diazaspiro[3.5]nonan-2-yl)benzenesulfonamide. LCMS calc. for C₄₅H₅₃N₁₀O₇S [M+H]⁺: m/z=877.4; Found: 877.2.

### Example 109. N-(6-((1H-Pyrazol-1-yl)methyl)-4-methoxybenzo[d]isoxazol-3-yl)-3-(7-((1-(2-(2,6-dioxopiperidin-3-yl)-1-methyl-1H-pyrrolo[2,3-b]pyridin-6-yl)piperidin-4-yl)methyl)-2,7-diazaspiro[3.5]nonan-2-yl)-5-fluoro-2,6-dimethoxybenzenesulfonamide

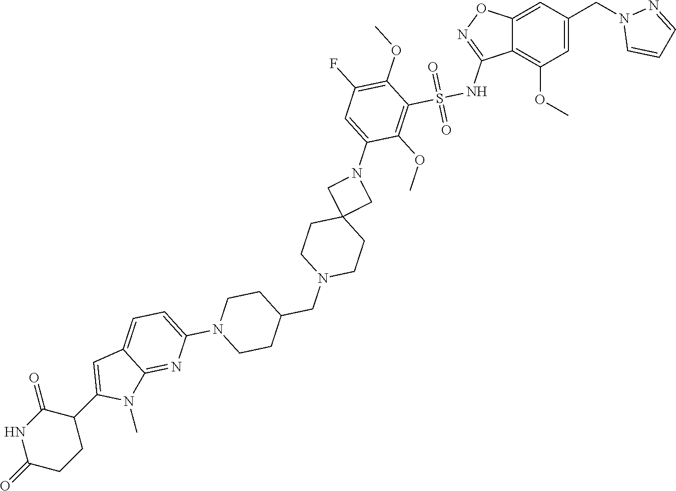 [SMILES: COc1c(F)cc(N2CC3(CCN(CC4CCN(c5ccc6cc(C7CCC(=O)NC7=O)n(C)c6n5)CC4)CC3)C2)c(OC)c1S(=O)(=O)Nc1noc2cc(Cn3cccn3)cc(OC)c12]

The title compound was synthesized by procedures analogous to that described in Example 2, using 1-(2-(2,6-dioxopiperidin-3-yl)-1-methyl-1H-pyrrolo[2,3-b]pyridin-6-yl)piperidine-4-carbaldehyde (from Example 108, Step 5) and N-(6-((1H-pyrazol-1-yl)methyl)-4-methoxybenzo[d]isoxazol-3-yl)-3-fluoro-2,6-dimethoxy-5-(2,7-diazaspiro[3.5]-nonan-2-yl)benzenesulfonamide (from Example 58, Step 4). LCMS calc. for C₄₆H₅₄FN₁₀O₈S [M+H]⁺: m/z=925.4; Found: 925.5.

### Example 110. N-(6-((1H-Pyrazol-1-yl)methyl)-4-methoxybenzo[d]isoxazol-3-yl)-3-(7-((1-(5-(2,6-dioxopiperidin-3-yl)pyridin-2-yl)piperidin-4-yl)methyl)-2,7-diazaspiro[3.5]nonan-2-yl)-5-fluoro-2,6-dimethoxybenzenesulfonamide

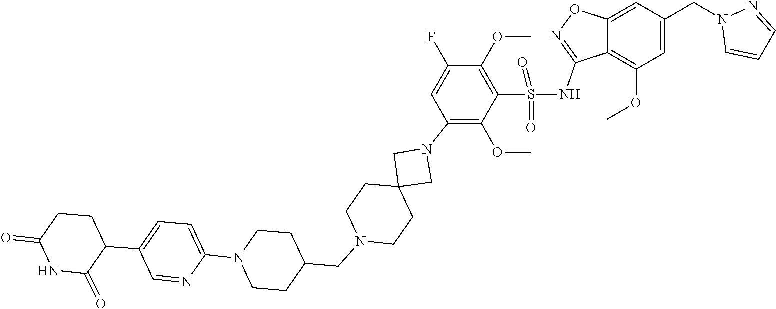 [SMILES: COc1c(F)cc(N2CC3(CCN(CC4CCN(c5ccc(C6CCC(=O)NC6=O)cn5)CC4)CC3)C2)c(OC)c1S(=O)(=O)Nc1noc2cc(Cn3cccn3)cc(OC)c12]

### Step 1. 5-Bromo-2-(4-(dimethoxymethyl)piperidin-1-yl)pyridine

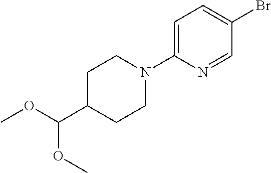 [SMILES: COC(OC)C1CCN(c2ccc(Br)cn2)CC1]

To a solution of 5-bromo-2-fluoropyridine (1.17 mL, 11.4 mmol) in DMSO (114 mL) was added 4-(dimethoxymethyl)piperidine (1.99 g, 12.5 mmol) and N,N-Diisopropyl-ethylamine (3.96 mL, 22.7 mmol). The reaction was stirred for 18 h at 90° C. . The mixture was poured into water and extracted with ethyl acetate. The combined organic layers were washed with brine, dried over Na₂SO₄, filtered, and concentrated. The crude material was purified by silica column chromatography (0-30% EtOAc/heptane) to the title compound (3.36 g, 10.7 mmol, 93.8% yield) as a white solid. LCMS calc. for C₁₃H₂₀BrN₂O₂ [M+H]⁺: m/z=315.1; Found: 315.4.

### Step 2. 2,6-Bis(benzyloxy)-6′-(4-(dimethoxymethyl)piperidin-1-yl)-3,3′-bipyridine

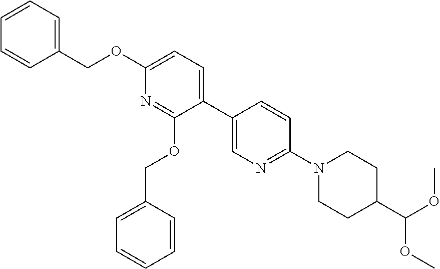 [SMILES: COC(OC)C1CCN(c2ccc(-c3ccc(OCc4ccccc4)nc3OCc3ccccc3)cn2)CC1]

The title compound was synthesized by procedures analogous to that described in Example 8, Step 2. LCMS calc. for C₃₂H₃₆N₃O₄ [M+H]⁺: m/z=526.3; Found: 526.6.

### Step 3. 3-(6-(4-(Dimethoxymethyl)piperidin-1-yl)pyridin-3-yl)piperidine-2,6-dione

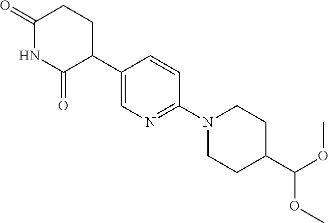 [SMILES: COC(OC)C1CCN(c2ccc(C3CCC(=O)NC3=O)cn2)CC1]

The title compound was synthesized by procedures analogous to that described in Example 1, Step 5. LCMS calc. for C₁₈H₂₆N₃O₄ [M+H]⁺: m/z=348.2; Found: 348.5.

### Step 4. 1-(5-(2,6-Dioxopiperidin-3-yl)pyridin-2-yl)piperidine-4-carbaldehyde

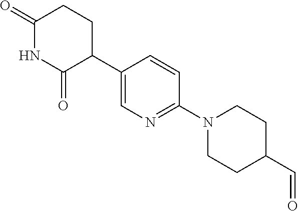 [SMILES: O=CC1CCN(c2ccc(C3CCC(=O)NC3=O)cn2)CC1]

The title compound was synthesized by procedures analogous to that described in Example 4, Step 4. LCMS calc. for C₁₆H₂₀N₃O₃ [M+H]⁺: m/z=302.1; Found: 302.1.

### Step 5. N-(6-((1H-Pyrazol-1-yl)methyl)-4-methoxybenzo[d]isoxazol-3-yl)-3-(7-((1-(5-(2,6-dioxopiperidin-3-yl)pyridin-2-yl)piperidin-4-yl)methyl)-2, 7-diazaspiro[3.5]-nonan-2-yl)-5-fluoro-2,6-dimethoxybenzenesulfonamide

The title compound was synthesized by procedures analogous to that described in Example 2, using N-(6-((1H-pyrazol-1-yl)methyl)-4-methoxybenzo[d]isoxazol-3-yl)-3-fluoro-2,6-dimethoxy-5-(2,7-diazaspiro[3.5]nonan-2-yl)benzenesulfonamide (from Example 58, Step 4). LCMS calc. for C₄₃H₅₁FN₉O₈S [M+H]⁺: m/z=872.4; Found: 872.5.

### Example 111. 4-(4-((2-(3-(N-(6-((1H-Pyrazol-1-yl)methyl)-4-methoxybenzo[d]isoxazol-3-yl)sulfamoyl)-5-fluoro-2,4-dimethoxyphenyl)-2,7-diazaspiro[3.5]nonan-7-yl)methyl)-piperidin-1-yl)-N-(2,6-dioxopiperidin-3-yl)benzamide

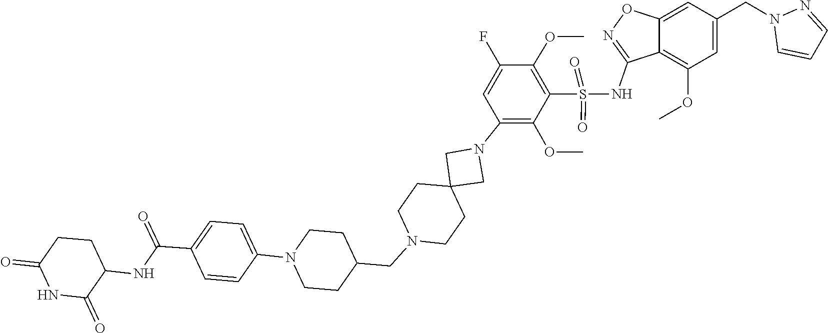 [SMILES: COc1c(F)cc(N2CC3(CCN(CC4CCN(c5ccc(C(=O)NC6CCC(=O)NC6=O)cc5)CC4)CC3)C2)c(OC)c1S(=O)(=O)Nc1noc2cc(Cn3cccn3)cc(OC)c12]

The title compound was synthesized by procedures analogous to that described in Example 2, using N-(6-((1H-pyrazol-1-yl)methyl)-4-methoxybenzo[d]isoxazol-3-yl)-3-fluoro-2,6-dimethoxy-5-(2,7-diazaspiro[3.5]nonan-2-yl)benzenesulfonamide (from Example 58, Step 4) and N-(2,6-dioxopiperidin-3-yl)-4-(4-formylpiperidin-1-yl)benzamide (which can be prepared as described in WO2025168131 A1). LCMS calc. for C₄₅H₅₃FN₉O₉S [M+H]⁺: m/z=914.4; Found: 914.4.

### Example 112. (R)—N-(6-((1H-Pyrazol-1-yl)methyl)-4-methoxybenzo[d]isoxazol-3-yl)-3-(7-((1-(4-(2,6-dioxopiperidin-3-yl-3,4,4,5,5-d5)phenyl)piperidin-4-yl)methyl)-2,7-diazaspiro[3.5]nonan-2-yl)-5-fluoro-2,6-dimethoxybenzenesulfonamide

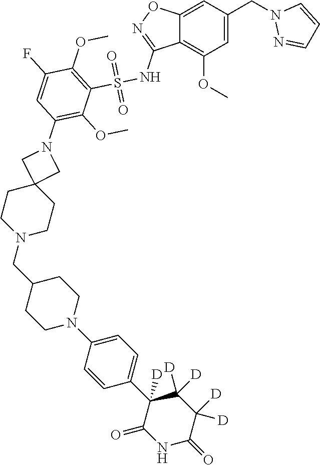 [SMILES: [2H]C1([2H])C(=O)NC(=O)[C@@]([2H])(c2ccc(N3CCC(CN4CCC5(CC4)CN(c4cc(F)c(OC)c(S(=O)(=O)Nc6noc7cc(Cn8cccn8)cc(OC)c67)c4OC)C5)CC3)cc2)C1([2H])[2H]]

### Step 1. 2,6-Bis(benzyloxy)-3-bromopyridine-4,5-d2

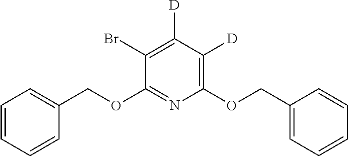 [SMILES: [2H]c1c(OCc2ccccc2)nc(OCc2ccccc2)c(Br)c1[2H]]

A mixture of 2,6-bis(benzyloxy)-3-bromopyridine (2.50 g, 6.75 mmol), cyclohexyl-(diphenyl)phosphane (906 mg, 3.38 mmol), deuterium oxide (2.71 g, 135 mmol), silver carbonate (373 mg, 1.35 mmol), and potassium carbonate (934 mg, 6.75 mmol) in toluene (0.60 mL) was heated at 120° C. in a sealed tube for 1 d. The reaction was cooled to ambient temperature and diluted with EtOAc (100 mL), washed with water (50 mL) and brine (50 mL), dried over Na₂SO₄, filtered, and concentrated. The residue was purified by silica gel chromatography (0-25% EtOAc/heptane) to afford the title compound (2111 mg, 5.67 mmol, 84.0%) as a light yellow solid. ¹H NMR (300 MHz, CDCl₃) δ 7.58-7.27 (m, 10H), 5.42 (s, 2H), 5.30 (s, 2H).

### Step 2. 2,6-Bis(benzyloxy)-3-(4-chlorophenyl)pyridine-4,5-d2

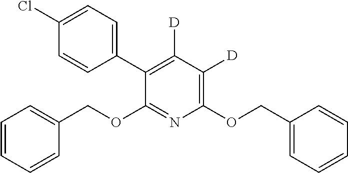 [SMILES: [2H]c1c(OCc2ccccc2)nc(OCc2ccccc2)c(-c2ccc(Cl)cc2)c1[2H]]

A mixture of (4-chlorophenyl)boronic acid (1.21 g, 7.76 mmol), 2,6-bis(benzyloxy)-3-bromopyridine-4,5-d₂ (1.93 g, 5.17 mmol), [1,1′-bis(diphenylphosphino)ferrocene]dichloro-palladium(II) (189 mg, 0.259 mmol), and cesium carbonate (5.06 g, 15.5 mmol) in 1,4-dioxane (20 mL) and water (4 mL) was heated at 90° C. under a nitrogen atmosphere for 2 h. The mixture was cooled to ambient temperature, diluted with EtOAc (150 mL), washed with water (75 mL) and brine (75 mL), dried over Na₂SO₄, filtered, and concentrated. The residue was purified by silica gel chromatography (0-20% EtOAc/heptane) to afford the title compound (1.94 mg, 4.81 mmol, 92.9%) as a yellow solid. C₂₆H₂₁D₂ClNO [M+H]⁺: m/z=404.1; Found: 404.2.

### Step 3. 2,6-Bis(benzyloxy)-3-(4-(4-(dimethoxymethyl)piperidin-1-yl)phenyl)pyridine-4,5-d₂

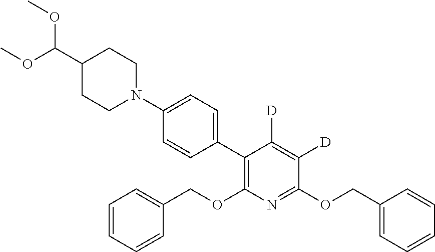 [SMILES: [2H]c1c(OCc2ccccc2)nc(OCc2ccccc2)c(-c2ccc(N3CCC(C(OC)OC)CC3)cc2)c1[2H]]

The title compound was synthesized by procedures analogous to that described in Example 4, Step 1. LCMS calc. for C₃₃H₃₅D₂N₂O₄[M+H]⁺: m/z=527.3; Found: 527.7.

### Step 4. (R)-3-(4-(4-(dimethoxymethyl)piperidin-1-yl)phenyl)piperidine-2,6-dione-3,4,4,5,5-d₅

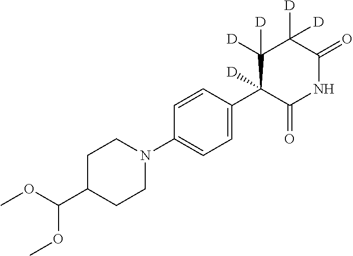 [SMILES: [2H]C1([2H])C(=O)NC(=O)[C@@]([2H])(c2ccc(N3CCC(C(OC)OC)CC3)cc2)C1([2H])[2H]]

2,6-Bis(benzyloxy)-3-(4-(4-(dimethoxymethyl)piperidin-1-yl)phenyl)pyridine-4,5-d₂ (1.84 g, 3.49 mmol was added into a solution of palladium on carbon (10% Pd, 372 mg, 3.49 mmol) in ethyl acetate (20 mL). The mixture was hydrogenated with 1 atm of deuterium gas (in balloon) for 18 h. The mixture was filtered, the filtrate was concentrated, and the crude material was purified by silica gel chromatography (0-75% EtOAc/DCM) to afford the racemic title compound (1.02 g, 2.89 mmol, 82.8%) as white solid. The enantiomers of the product were separated on a Lux Cellulose-3 column (10:45:45 hexanes/MeOH/isopropanol) (Peak 1 ((R)-isomer)=5.1 min, Peak 2 ((S)-isomer)=7.2 min) to afford the single enantiomer of the title compound (20 mg, 50% yield, >99% ee). C₁₉H₂₂D₅N₂O₄[M+H]⁺: m/z=352.2; Found: 352.5.

### Step 5. (R)-1-(4-(2,6-Dioxopiperidin-3-yl-3,4,4,5,5-d5)phenyl)piperidine-4-carbaldehyde

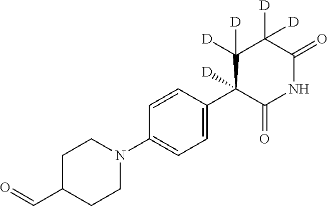 [SMILES: [2H]C1([2H])C(=O)NC(=O)[C@@]([2H])(c2ccc(N3CCC(C=O)CC3)cc2)C1([2H])[2H]]

The title compound was synthesized by procedures analogous to that described in Example 4, Step 4. LCMS calc. for C₁₇H₁₆D₅N₂O₃[M+H]⁺: m/z=306.2; Found: 306.2.

### Step 6. (R)—N-(6-((1H-Pyrazol-1-yl)methyl)-4-methoxybenzo[d]isoxazol-3-yl)-3-(7-((1-(4-(2,6-dioxopiperidin-3-yl-3,4,4,5,5-d5)phenyl)piperidin-4-yl)methyl)-2, 7-diazaspiro-[3.5]nonan-2-yl)-5-fluoro-2,6-dimethoxybenzenesulfonamide

The title compound was synthesized by procedures analogous to that described in Example 2, using N-(6-((1H-pyrazol-1-yl)methyl)-4-methoxybenzo[d]isoxazol-3-yl)-3-fluoro-2,6-dimethoxy-5-(2,7-diazaspiro[3.5]nonan-2-yl)benzenesulfonamide (from Example 113, Step 2). LCMS calc. for C₄₄H₄₇D₅FN₈O₈S [M+H]⁺: m/z=876.4; Found: 876.4. Chiral purity assessed using a (S,S)Whelk-O1 column (ethanol)=98.1% ee.

### Example 113. (R)—N-(6-((1H-Pyrazol-1-yl)methyl)-4-methoxybenzo[d]isoxazol-3-yl)-3-(7-((1-(4-(2,6-dioxopiperidin-3-yl)phenyl)piperidin-4-yl)methyl)-2,7-diazaspiro[3.5]nonan-2-yl)-5-fluoro-2,6-dimethoxybenzenesulfonamide

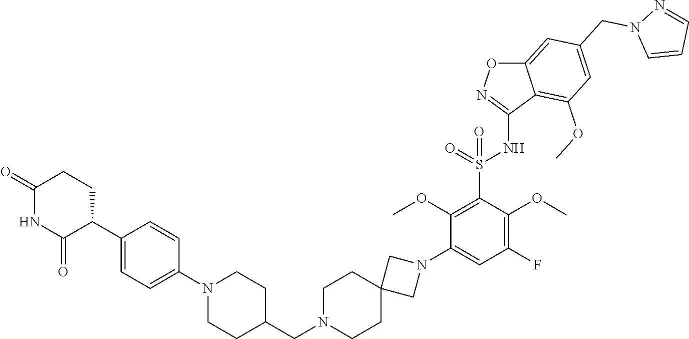 [SMILES: COc1c(F)cc(N2CC3(CCN(CC4CCN(c5ccc([C@H]6CCC(=O)NC6=O)cc5)CC4)CC3)C2)c(OC)c1S(=O)(=O)Nc1noc2cc(Cn3cccn3)cc(OC)c12]

### Step 1. N-(6-((1H-Pyrazol-1-yl)methyl)-4-methoxybenzo[d]isoxazol-3-yl)-3-bromo-5-fluoro-2,6-dimethoxybenzenesulfonamide

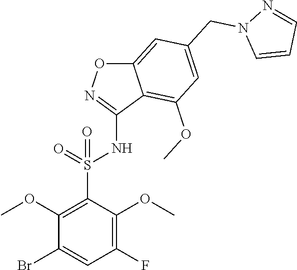 [SMILES: COc1c(F)cc(Br)c(OC)c1S(=O)(=O)Nc1noc2cc(Cn3cccn3)cc(OC)c12]

To a solution of 6-((1H-pyrazol-1-yl)methyl)-4-methoxybenzo[d]isoxazol-3-amine (355 mg, 1.45 mmol) (can be prepared as described in WO2023245150A1) and 3-bromo-5-fluoro-2,6-dimethoxybenzenesulfonyl chloride (from 55, Step 2) (1.21 g, 3.63 mmol) in THF (15 mL) was added potassium tert-butoxide (1.30 g, 11.6 mmol). The reaction was stirred for 15 min. The reaction was quenched with 1N HCl (aq.) adjusting the pH to around 3, and then the reaction mixture was diluted with water (20 mL) and EtOAc (20 mL). The aqueous layer was extracted with EtOAc (3×15 mL). The combined organic layers were washed with brine, dried over MgSO₄, filtered, and concentrated. Purification by silica gel chromatography (0-10% MeOH/DCM) afforded the title compound (676 mg, 1.25 mmol, 85.9% yield) as a tan solid. LCMS calc. for C₂₀H₁₉BrFN₄O₆S [M+H]⁺: m/z=541.0, 543.0; Found: 541.0, 543.0.

### Step 2. N-(6-((1H-Pyrazol-1-yl)methyl)-4-methoxybenzo[d]isoxazol-3-yl)-3-fluoro-2,6-dimethoxy-5-(2, 7-diazaspiro[3.5]nonan-2-yl)benzenesulfonamide

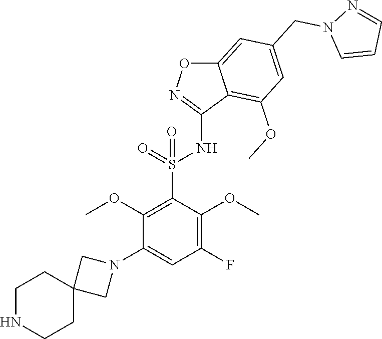 [SMILES: COc1c(F)cc(N2CC3(CCNCC3)C2)c(OC)c1S(=O)(=O)Nc1noc2cc(Cn3cccn3)cc(OC)c12]

To a nitrogen-sparged mixture of N-(6-((1H-pyrazol-1-yl)methyl)-4-methoxybenzo-[d]isoxazol-3-yl)-3-bromo-5-fluoro-2,6-dimethoxybenzenesulfonamide (50.0 mg, 0.0924 mmol), sodium tert-butoxide (26.6 mg, 0.277 mmol), and tert-butyl 2,7-diazaspiro[3.5]-nonane-7-carboxylate (25.1 mg, 0.111 mmol) in 1,4-dioxane (1 mL) was added BrettPhos Pd G3 (CAS: 1470372-59-8, 8.37 mg, 0.0924 mmol). The mixture was sparged with N₂ an additional 3 min and then stirred at 100° C. for 2 h. The reaction was cooled to room temperature and concentrated. TFA (1.5 mL) was added, and the mixture was stirred for 1 h. The mixture was concentrated and purified by silica gel chromatography (0-20% MeOH/DCM) to afford the title compound (59 mg, 0.10 mmol, >99%) as a white solid. LCMS calc. for C₂₇H₃₂FN₆O₆S [M+H]⁺: m/z=587.2; Found: 587.2.

### Step 3. (R)-3-(4-(4-(Dimethoxymethyl)piperidin-1-yl)phenyl)piperidine-2,6-dione and (S)-3-(4-(4-(Dimethoxymethyl)piperidin-1-yl)phenyl)piperidine-2,6-dione

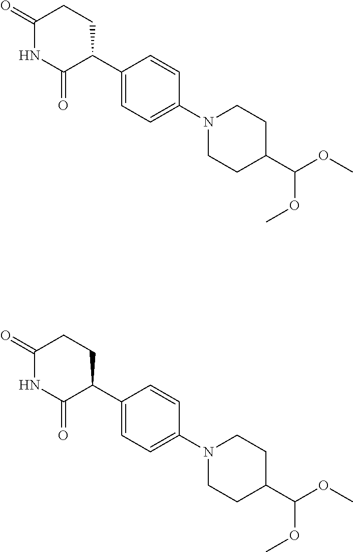 [SMILES: COC(OC)C1CCN(c2ccc([C@H]3CCC(=O)NC3=O)cc2)CC1 ; COC(OC)C1CCN(c2ccc([C@@H]3CCC(=O)NC3=O)cc2)CC1]

Chiral separation of 3-(4-(4-(dimethoxymethyl)piperidin-1-yl)phenyl)piperidine-2,6-dione (1.00 g, 2.89 mmol) (which can be prepared as described in WO202403991A₂) using a REGIS (S,S) WHELK-O 1 column (Isocratic elution, 60% IPA+MeCN in supercritical CO₂) (Peak 1 ((R)-isomer)=4.3 min, Peak 2 ((S)-isomer)=6.6 min) afforded the title compound as the (R)-isomer (492 mg, 1.42 mmol, 49.1% yield, 99.5% ee). LCMS calc. for C₁₉H₂₇N₂O₄ [M+H]⁺: m/z=347.2; Found: 347.3. The (S)-isomer was also collected (487 mg, 1.41 mmol, 49% yield, >99% ee). LCMS calc. for C₁₉H₂₇N₂O₄ [M+H]⁺: m/z=347.2; Found: 347.3.

### Step 4. (R)-1-(4-(2,6-Dioxopiperidin-3-yl)phenyl)piperidine-4-carbaldehyde

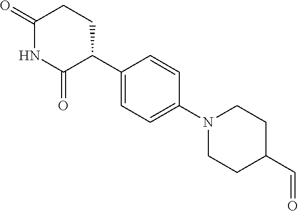 [SMILES: O=CC1CCN(c2ccc([C@H]3CCC(=O)NC3=O)cc2)CC1]

To a slurry of (R)-3-(4-(4-(dimethoxymethyl)piperidin-1-yl)phenyl)piperidine-2,6-dione (100 mg, 0.289 mmol) in DCM (0.5 mL) and acetone (0.5 mL) was added TFA (111 mL, 1.45 mmol) dropwise. The mixture was stirred for 6 h and then concentrated. To the resulting material was added NaHCO₃ (aq.) to adjust to pH 8-9. The resulting solid was collected by filtration to afford the title compound (65 mg, 0.22 mmol, 75% yield, 99% ee) as an off-white solid. LCMS calc. for C₁₇H₂₁N₂O₃ [M+H]⁺: m/z=301.2; Found: 301.3.

### Step 5. (R)—N-(6-((1H-Pyrazol-1-yl)methyl)-4-methoxybenzo[d]isoxazol-3-yl)-3-(7-((1-(4-(2,6-dioxopiperidin-3-yl)phenyl)piperidin-4-yl)methyl)-2,7-diazaspiro[3.5]nonan-2-yl)-5-fluoro-2,6-dimethoxybenzenesulfonamide

To a solution of N-(6-((1H-pyrazol-1-yl)methyl)-4-methoxybenzo[d]isoxazol-3-yl)-3-fluoro-2,6-dimethoxy-5-(2,7-diazaspiro[3.5]nonan-2-yl)benzenesulfonamide (100 mg, 0.17 mmol), (R)-1-(4-(2,6-dioxopiperidin-3-yl)phenyl)piperidine-4-carbaldehyde (61 mg, 0.20 mmol), and acetic acid (44 mg, 0.34 mmol) in DMSO (2 mL) was added sodium triacetoxy-borohydride (110 mg, 0.51 mmol) portion-wise at 10-20° C. The mixture was stirred at 15-25° C. for 1-2 h. The reaction mixture was poured into water (3 mL) and filtered. The filter cake was washed with water (0.5 mL) and MTBE (0.5 mL) and dried at 40° C. for 16 h. The solid was triturated with 4:1 water/MeCN (20 mL) for 2-3 h and then filtered and dried. The solid was dispersed with 4:1 water/MeCN/water (10 mL), and H₂SO₄ (1 M, 0.17 mL) was added. The resulting solution was filtered and concentrated. The resulting solid was triturated with IPA (1 mL) for 1 h, then filtered and dried to afford the title compound as the H₂SO₄ salt (92 mg, 0.095 mmol, 56% yield, 88.5% ee). ¹H NMR (400 MHz, DMSO-d₆) δ 10.79 (s, 1H), 10.36 (s, 1H), 8.87 (s, 1H), 7.89 (d, J=2.3 Hz, 1H), 7.51 (d, J=1.8 Hz, 1H), 7.06 (d, J=8.3 Hz, 2H), 6.93 (d, J=8.3 Hz, 2H), 6.87 (s, 1H), 6.77 (d, J=0.9 Hz, 1H), 6.73 (d, J=12.9 Hz, 1H), 6.31 (t, J=2.1 Hz, 1H), 5.46 (s, 2H), 3.81 (s, 3H), 3.75 (s, 3H), 3.73-3.72 (m, 2H), 3.69-3.67 (m, 3H), 3.62 (s, 3H), 3.57 (s, 2H), 3.46 (d, J=12.3 Hz, 2H), 3.03-2.94 (m, 4H), 2.78-2.58 (m, 3H), 2.49-2.41 (m, 1H), 2.21-2.05 (m, 3H), 2.04-1.87 (m, 3H), 1.81 (d, J=12.5 Hz, 2H), 1.33 (q, J=10.7 Hz, 2H). LCMS calc. for C₄₄H₅₂FN₈O₈S [M+H]⁺: m/z=871.4; Found: 871.6.

### Example 114. (S)—N-(6-((1H-Pyrazol-1-yl)methyl)-4-methoxybenzo[d]isoxazol-3-yl)-3-(7-((1-(4-(2,6-dioxopiperidin-3-yl)phenyl)piperidin-4-yl)methyl)-2,7-diazaspiro[3.5]nonan-2-yl)-5-fluoro-2,6-dimethoxybenzenesulfonamide

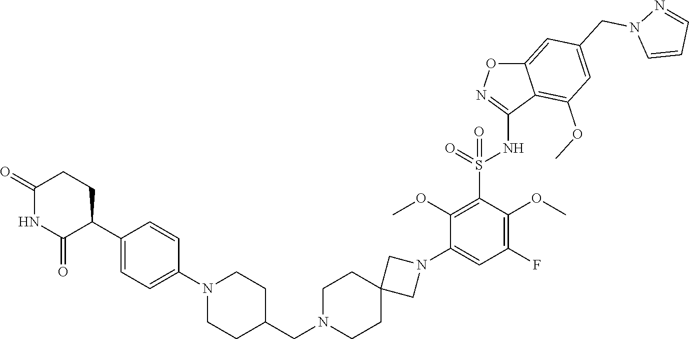 [SMILES: COc1c(F)cc(N2CC3(CCN(CC4CCN(c5ccc([C@@H]6CCC(=O)NC6=O)cc5)CC4)CC3)C2)c(OC)c1S(=O)(=O)Nc1noc2cc(Cn3cccn3)cc(OC)c12]

The title compound was synthesized according to procedures analogous to Example 113 substituting (S)-3-(4-(4-(Dimethoxymethyl)piperidin-1-yl)phenyl)piperidine-2,6-dione (from Step 3) for (R)-3-(4-(4-(Dimethoxymethyl)piperidin-1-yl)phenyl)piperidine-2,6-dione in Step 4. ¹H NMR (600 MHz, DMSO-d6) δ 10.77 (s, 1H), 7.84 (s, 1H), 7.46 (s, 1H), 7.01 (d, J=8.2 Hz, 2H), 6.86 (d, J=8.3 Hz, 2H), 6.60 (s, 1H), 6.50 (d, J=11.8 Hz, 1H), 6.27 (s, 1H), 5.38 (s, 2H), 3.78 (s, 3H), 3.70 (dd, J=10.9, 4.6 Hz, 1H), 3.64 (d, J=10.1 Hz, 5H), 3.56 (s, 3H), 3.51 (s, 4H), 2.74 (s, 3H), 2.61 (t, J=11.7 Hz, 5H), 2.44 (d, J=17.4 Hz, 1H), 2.14-2.05 (m, 2H), 1.98 (dd, J=12.9, 4.7 Hz, 1H), 1.83 (s, 4H), 1.76 (d, J=11.4 Hz, 3H), 1.21 (d, J=10.4 Hz, 2H). LCMS calc. for C₄₄H₅₂FN₈O₈S [M+H]⁺: m/z=871.4; Found: 871.6. Chiral purity: 92.0% ee.

### Example 115. N-(6-((1H-Pyrazol-1-yl)methyl)-4-methoxybenzo[d]isoxazol-3-yl)-3-(6-((1-(3-(2,6-dioxopiperidin-3-yl)-1-(methyl-d3)-1H-pyrrolo[2,3-b]pyridin-6-yl)piperidin-4-yl)methyl)-5,6,7,8-tetrahydro-1,6-naphthyridin-2-yl)-5-fluoro-2,6-dimethoxybenzene-sulfonamide

### Step 1. 6-Bromo-3-iodo-1H-indazole

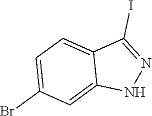 [SMILES: Brc1ccc2c(I)n[nH]c2c1]

To a solution of 6-bromoindazole (6.00 g, 30.5 mmol) in DMSO (100 mL) was added 1-iodopyrrole-2,5-dione (8.83 g, 39.6 mmol). The mixture was stirred at 90° C. for 4 h. The mixture was poured into cold water and extracted with ethyl acetate. The organic layers were dried over anhydrous sodium sulfate, filtered, and concentrated. The crude material was purified by silica gel chromatography (0-20% EtOAc/heptane) to afford the title compound (6.00 g, 18.6 mmol, 61.0%) as a light pink solid. LCMS calc. for C₇H₅BrIN₂ [M+H]⁺ m/z=322.9; Found 323.1.

### Step 2: 6-Bromo-3-iodo-1-(methyl-d3)-1H-indazole

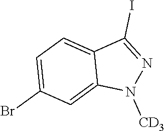 [SMILES: [2H]C([2H])([2H])n1nc(I)c2ccc(Br)cc21]

To a nitrogen sparged solution of 6-bromo-3-iodo-1H-indazole (2.00 g, 6.19 mmol) and cesium carbonate (5.04 g, 15.5 mmol) in DMF (20 mL) was added trideuteriomethyl trifluoromethanesulfonate (2.59 g, 15.5 mmol) dropwise at 0° C. The mixture was stirred at 0° C. for 4 h, then poured in cold water and extracted with MTBE. The organic layers were dried over anhydrous sodium sulfate, filtered, and concentrated. The crude material was purified by silica gel chromatography (0-20% MTBE/heptane) to afford the title compound (1.87 g, 5.49 mmol, 88.76%) as an orange-red solid. LCMS calc. for C₈H₄D₃BrIN₂ [M+H]+m/z=339.9; Found 340.1.

### Step 3: 1-(6-Bromo-1-(methyl-d3)-1H-indazol-3-yl)dihydropyrimidine-2,4(1H,3H)-dione

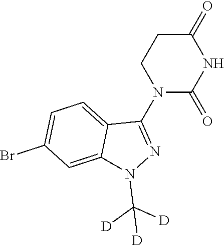 [SMILES: [2H]C([2H])([2H])n1nc(N2CCC(=O)NC2=O)c2ccc(Br)cc21]

The title compound was synthesized by procedures analogous to those outlined in Example 92, Steps 2-3. LCMS calc. for C₁₂H₉D₃BrN₄O₂[M+H]⁺ m/z=326.0; Found 326.2.

### Step 4. 1-(6-(4-(Dimethoxymethyl)piperidin-1-yl)-1-(methyl-d3)-1H-indazol-3-yl)dihydropyrimidine-2,4(1H,3H)-dione

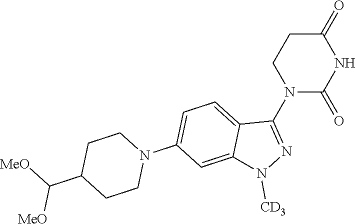 [SMILES: [2H]C([2H])([2H])n1nc(N2CCC(=O)NC2=O)c2ccc(N3CCC(C(OC)OC)CC3)cc21]

The title compound was synthesized by procedures analogous to those outlined in Example 4, Step 1. LCMS calc. for C₂₀H₂₅D₃N₅O₄[M+H]⁺ m/z=405.2; Found 405.4.

### Step 5. 1-(3-(2,4-Dioxotetrahydropyrimidin-1(2H)-yl)-1-(methyl-d3)-1H-indazol-6-yl)piperidine-4-carbaldehyde

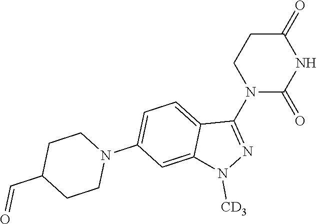 [SMILES: [2H]C([2H])([2H])n1nc(N2CCC(=O)NC2=O)c2ccc(N3CCC(C=O)CC3)cc21]

The title compound was synthesized by procedures analogous to those outlined in Example 4, Step 4. LCMS calc. for C₁₈H₁₉D₃N₅O₃[M+H]⁺ m/z=359.2; Found 359.4.

### Step 6: N-(6-((1H-Pyrazol-1-yl)methyl)-4-methoxybenzo[d]isoxazol-3-yl)-3-(6-((1-(3-(2,6-dioxopiperidin-3-yl)-1-(methyl-d3)-1H-pyrrolo[2,3-b]pyridin-6-yl)piperidin-4-yl)methyl)-5,6,7,8-tetrahydro-1, 6-naphthyridin-2-yl)-5-fluoro-2,6-dimethoxybenzene-sulfonamide

The title compound was synthesized by procedures analogous to those outlined in Example 2, using N-(6-((1H-pyrazol-1-yl)methyl)-4-methoxybenzo[d]isoxazol-3-yl)-3-fluoro-2,6-dimethoxy-5-(5,6,7,8-tetrahydro-1,6-naphthyridin-2-yl)benzenesulfonamide (from Example % SRZ14, Step 3). LCMS calc. for C₄₇H₄₇D₃FN₁₀O₈S [M+H]⁺ m/z=936.4; Found 936.8.

### Example 116. N-(6-((1H-Pyrazol-1-yl)methyl)-4-methoxybenzo[d]isoxazol-3-yl)-3-(6-((1-(3-(2,4-dioxotetrahydropyrimidin-1(2H)-yl)-1-(methyl-d3)-1H-indazol-6-yl)piperidin-4-yl)methyl)-5,6,7,8-tetrahydro-1,6-naphthyridin-2-yl)-5-fluoro-2,6-dimethoxybenzenesulfonamide

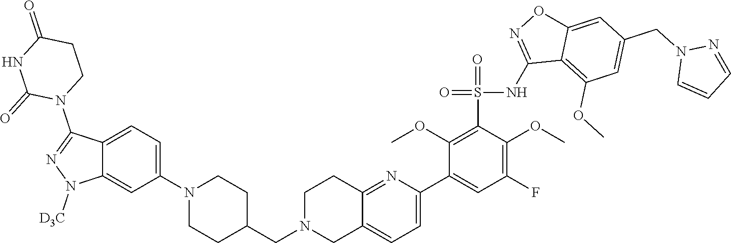 [SMILES: [2H]C([2H])([2H])n1nc(N2CCC(=O)NC2=O)c2ccc(N3CCC(CN4CCc5nc(-c6cc(F)c(OC)c(S(=O)(=O)Nc7noc8cc(Cn9cccn9)cc(OC)c78)c6OC)ccc5C4)CC3)cc21]

### Step 1. 6-Chloro-3-iodo-1H-pyrrolo[2,3-b]pyridine

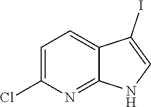 [SMILES: Clc1ccc2c(I)c[nH]c2n1]

The title compound was synthesized by procedures analogous to those outlined in Example 115, Step 1. LCMS calc. for C₇H₅ClIN₂ [M+H]⁺ m/z=278.9; Found 279.2.

### Step 2: 6-Chloro-3-iodo-1-(methyl-d3)-1H-pyrrolo[2,3-b]pyridine

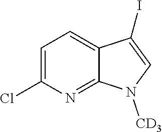 [SMILES: [2H]C([2H])([2H])n1cc(I)c2ccc(Cl)nc21]

To a solution of 6-chloro-3-iodo-1H-pyrrolo[2,3-b]pyridine (8.00 g, 28.7 mmol) and cesium carbonate (2.34 g, 71.8 mmol) in DMSO (100 mL) was added iodomethane-d₃ (1.04 g, 71.8 mmol). The reaction mixture was stirred for 2 h, then poured into water and extracted with ethyl acetate. The organic layer was dried over anhydrous sodium sulfate, filtered, and concentrated. The crude material was purified by silica gel chromatography (0-40% of MTBE/heptane) to afford the title compound (8.00 g, 27.1 mmol, 94.3%) as light pink solid. LCMS calc. for C₈H₄D₃ClIN₂ [M+H]⁺ m/z=296.0; Found 296.2.

### Step 3. 3-(2,6-Bis(benzyloxy)pyridin-3-yl)-6-chloro-1-(methyl-d3)-1H-pyrrolo[2,3-b]pyridine

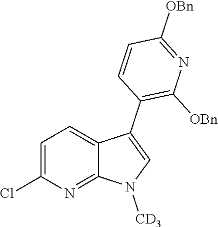 [SMILES: [2H]C([2H])([2H])n1cc(-c2ccc(OCc3ccccc3)nc2OCc2ccccc2)c2ccc(Cl)nc21]

The title compound was synthesized by procedures analogous to those outlined in Example 8, Step 2. LCMS calc. for C₂₇H₂₀D₃ClN₃O₂[M+H]⁺ m/z=459.2; Found 459.3.

### Step 4: 3-(2,6-Bis(benzyloxy)pyridin-3-yl)-6-(4-(dimethoxymethyl)piperidin-1-yl)-1-(methyl-d3)-1H-pyrrolo[2,3-b]pyridine

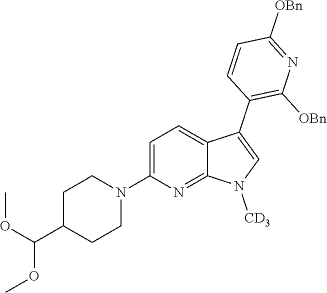 [SMILES: [2H]C([2H])([2H])n1cc(-c2ccc(OCc3ccccc3)nc2OCc2ccccc2)c2ccc(N3CCC(C(OC)OC)CC3)nc21]

The title compound was synthesized by procedures analogous to those outlined in Example 100, Step 2. LCMS calc. for C₃₅H₃₆D₃N₄O₄[M+H]⁺ m/z=582.3; Found 582.6.

### Step 5: 3-(6-(4-(Dimethoxymethyl)piperidin-1-yl)-1-(methyl-d3)-1H-pyrrolo[2,3-b]pyridin-3-yl)piperidine-2,6-dione

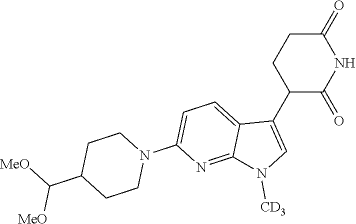 [SMILES: [2H]C([2H])([2H])n1cc(C2CCC(=O)NC2=O)c2ccc(N3CCC(C(OC)OC)CC3)nc21]

The title compound was synthesized by procedures analogous to those outlined in Example 1, Step 5. LCMS calc. for C₂₁H₂₆D₃N₄O₄[M+H]⁺ m/z=404.2; Found 404.4.

### Step 6: 1-(3-(2,6-Dioxopiperidin-3-yl)-1-(methyl-d3)-1H-pyrrolo[2,3-b]pyridin-6-yl)piperidine-4-carbaldehyde

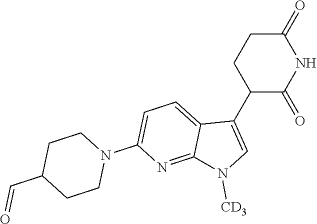 [SMILES: [2H]C([2H])([2H])n1cc(C2CCC(=O)NC2=O)c2ccc(N3CCC(C=O)CC3)nc21]

The title compound was synthesized by procedures analogous to those outlined in Example 4, Step 4. LCMS calc. for C₁₉H₂₀D₃N₄O₃[M+H]⁺ m/z=358.2; Found 358.4.

### Step 7. N-(6-((1H-Pyrazol-1-yl)methyl)-4-methoxybenzo[d]isoxazol-3-yl)-3-(6-((1-(3-(2,4-dioxotetrahydropyrimidin-1(2H)-yl)-1-(methyl-d3)-1H-indazol-6-yl)piperidin-4-yl)methyl)-5,6,7,8-tetrahydro-1, 6-naphthyridin-2-yl)-5-fluoro-2,6-dimethoxybenzene-sulfonamide

The title compound was synthesized by procedures analogous to those outlined in Example 2, using N-(6-((1H-pyrazol-1-yl)methyl)-4-methoxybenzo[d]isoxazol-3-yl)-3-fluoro-2,6-dimethoxy-5-(5,6,7,8-tetrahydro-1,6-naphthyridin-2-yl)benzenesulfonamide (from Example 61, Step 3). LCMS calc. for C₄₆H₄₆D₃FN₁₁O₈S [M+H]⁺ m/z=937.4; Found 937.7.

### Example 117. N-(6-((1H-Pyrazol-1-yl)methyl)-4-methoxybenzo[d]isoxazol-3-yl)-3-(7-((1-(3-(2,6-dioxopiperidin-3-yl)-1-(2,2,2-trifluoroethyl)-1H-indazol-6-yl)piperidin-4-yl)methyl)-2,7-diazaspiro[3.5]nonan-2-yl)-5-fluoro-2,6-dimethoxybenzenesulfonamide

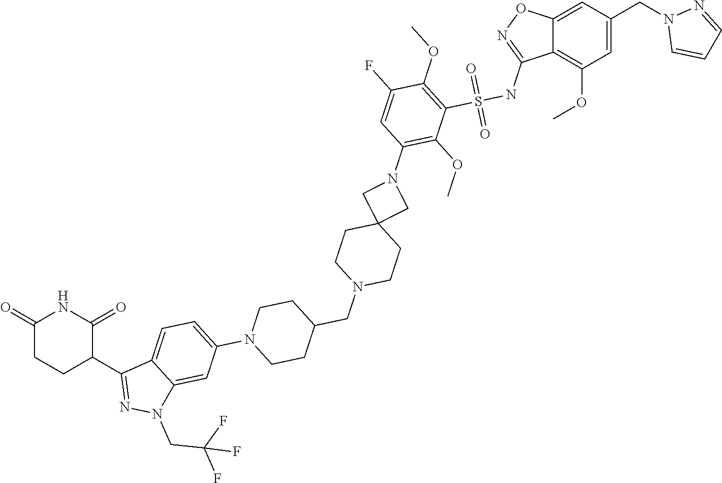 [SMILES: COc1c(F)cc(N2CC3(CCN(CC4CCN(c5ccc6c(C7CCC(=O)NC7=O)nn(CC(F)(F)F)c6c5)CC4)CC3)C2)c(OC)c1S(=O)(=O)Nc1noc2cc(Cn3cccn3)cc(OC)c12]

### Step 1. 6-Bromo-3-iodo-1-(2,2,2-trifluoroethyl)-1H-indazole

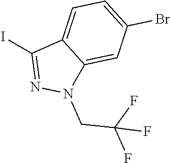 [SMILES: FC(F)(F)Cn1nc(I)c2ccc(Br)cc21]

To a nitrogen sparged solution of 6-bromo-3-iodo-1H-indazole (1000 mg, 3.10 mmol) and cesium carbonate (3030 mg, 9.29 mmol) in DMF (8 mL) and DMSO (8 mL) was added 2,2,2-trifluoroethyl trifluoromethanesulfonate (862 mg, 3.72 mmol) at 0° C. dropwise. The mixture was stirred for 1 h at 0° C., then quenched with cold water and extracted with ethyl acetate. The organic layers were washed with water and brine, dried over anhydrous sodium sulfate, filtered, and concentrated. The crude material was purified by silica gel chromatography (0-20% EtOAc/heptane) to afford the title compound (1170 mg, 2.90 mmol, 93.6%) as a white solid. LCMS calc. for C₉H₆BrF₃IN₂ [M+H]⁺: m/z=404.9, 406.9; Found: 405.0, 406.9.

### Step 2. 3-(2,6-Bis(benzyloxy)pyridin-3-yl)-6-bromo-1-(2,2,2-trifluoroethyl)-1H-indazole

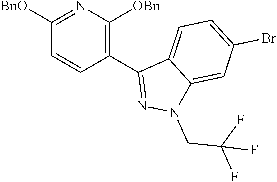 [SMILES: FC(F)(F)Cn1nc(-c2ccc(OCc3ccccc3)nc2OCc2ccccc2)c2ccc(Br)cc21]

The title compound was synthesized according to procedures analogous to Example 58, Step 1. LCMS calc. for C₂₈H₂₂BrF₃N₃O₂ [M+H]⁺: m/z=568.1, 570.1; Found: 568.1, 570.0.

### Step 3. 3-(2,6-Bis(benzyloxy)pyridin-3-yl)-6-(4-(dimethoxymethyl)piperidin-1-yl)-1-(2,2,2-trifluoroethyl)-1H-indazole

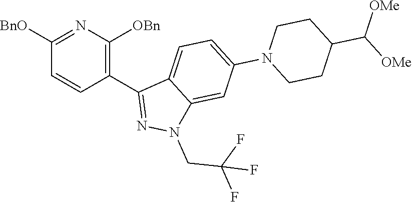 [SMILES: COC(OC)C1CCN(c2ccc3c(-c4ccc(OCc5ccccc5)nc4OCc4ccccc4)nn(CC(F)(F)F)c3c2)CC1]

The title compound was synthesized according to procedures analogous to Example 4, Step 1. LCMS calc. for C₃₆H₃₈F₃N₄O₄[M+H]⁺: m/z=647.3; Found: 647.4.

### Step 4. 1-(3-(2,6-Dioxopiperidin-3-yl)-1-(2,2,2-trifluoroethyl)-1H-indazol-6-yl)piperidine-4-carbaldehyde

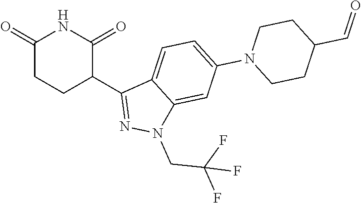 [SMILES: O=CC1CCN(c2ccc3c(C4CCC(=O)NC4=O)nn(CC(F)(F)F)c3c2)CC1]

The title compound was synthesized according to procedures analogous to Example 4, Steps 3-4. LCMS calc. for C₂₀H₂₂F₃N₄O₃[M+H]⁺: m/z=423.2; Found: 423.2.

### Step 5. N-(6-((1H-Pyrazol-1-yl)methyl)-4-methoxybenzo[d]isoxazol-3-yl)-3-(7-((1-(3-(2,6-dioxopiperidin-3-yl)-1-(2,2,2-trifluoroethyl)-1H-indazol-6-yl)piperidin-4-yl)methyl)-2, 7-diazaspiro[3.5]nonan-2-yl)-5-fluoro-2,6-dimethoxybenzenesulfonamide

The title compound was synthesized according to procedures analogous to Example 2, using N-(6-((1H-pyrazol-1-yl)methyl)-4-methoxybenzo[d]isoxazol-3-yl)-3-fluoro-2,6-dimethoxy-5-(2,7-diazaspiro[3.5]nonan-2-yl)benzenesulfonamide (from Example 113, Step 2). ¹H NMR (400 MHz, CH₃OD) δ 7.80 (dd, J=2.3, 0.7 Hz, 1H), 7.64 (d, J=8.9 Hz, 1H), 7.59 (dd, J=2.0, 0.7 Hz, 1H), 7.14-6.99 (m, 2H), 6.79 (d, J=0.9 Hz, 1H), 6.73-6.63 (m, 2H), 6.39 (t, J=2.2 Hz, 1H), 5.49 (s, 2H), 5.18-5.04 (m, 2H), 4.35 (dd, J=8.8, 5.1 Hz, 1H), 3.98 (s, 3H), 3.94-3.85 (m, 5H), 3.80-3.72 (m, 5H), 3.68-3.58 (m, 4H), 3.19-3.01 (m, 4H), 2.94 (t, J=12.1 Hz, 2H), 2.88-2.64 (m, 2H), 2.46 (ddt, J=13.8, 8.7, 4.3 Hz, 1H), 2.34 (dq, J=12.2, 5.6 Hz, 1H), 2.23 (d, J=14.3 Hz, 2H), 2.19-1.89 (m, 5H), 1.56 (q, J=10.9 Hz, 2H). LCMS calc. for C₄₇H₅₃F₄N₁₀O₈S [M+H]⁺: m/z=993.4; Found: 993.3.

### Example 120. N-(6-((1H-Pyrazol-1-yl)methyl)-4-methoxybenzo[d]isoxazol-3-yl)-3-(7-(3-(2,6-dioxopiperidin-3-yl)-1-methyl-1H-indazol-6-yl)-2,7-diazaspiro[3.5]nonan-2-yl)-2-methoxybenzenesulfonamide

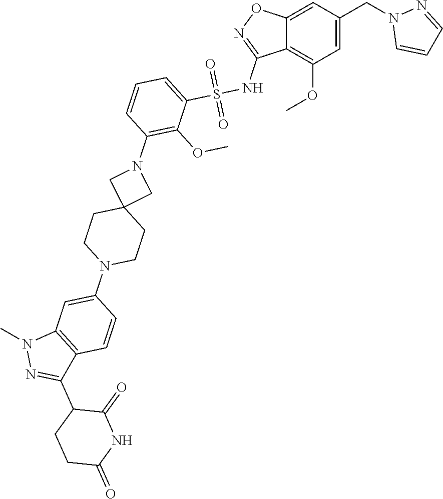 [SMILES: COc1c(N2CC3(CCN(c4ccc5c(C6CCC(=O)NC6=O)nn(C)c5c4)CC3)C2)cccc1S(=O)(=O)Nc1noc2cc(Cn3cccn3)cc(OC)c12]

### Step 1. tert-butyl 7-(3-(2,6-Bis(benzyloxy)pyridin-3-yl)-1-methyl-1H-indazol-6-yl)-2, 7-diazaspiro[3.5]nonane-2-carboxylate

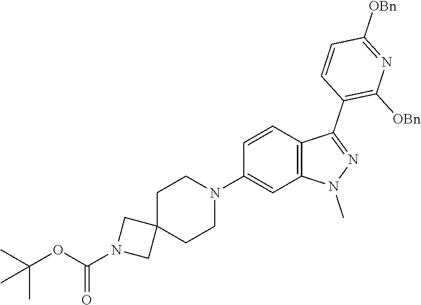 [SMILES: Cn1nc(-c2ccc(OCc3ccccc3)nc2OCc2ccccc2)c2ccc(N3CCC4(CC3)CN(C(=O)OC(C)(C)C)C4)cc21]

The title compound was synthesized by procedures analogous to those outlined in Example 58, Step 2, using tert-butyl 2,7-diazaspiro[3.5]nonane-2-carboxylate. LCMS calc. for C₃₉H₄₄N₅O₄ [M+H]⁺ m/z=646.3; Found 646.2.

### Step 2. tert-butyl 7-(3-(2,6-Dioxopiperidin-3-yl)-1-methyl-1H-indazol-6-yl)-2, 7-diazaspiro[3.5]nonane-2-carboxylate

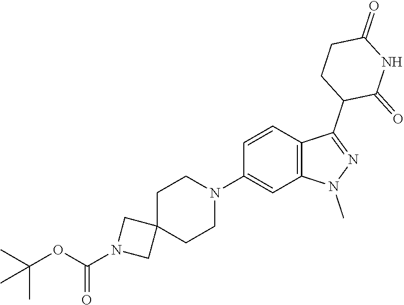 [SMILES: Cn1nc(C2CCC(=O)NC2=O)c2ccc(N3CCC4(CC3)CN(C(=O)OC(C)(C)C)C4)cc21]

The title compound was synthesized by procedures analogous to those outlined in Example 1, Step 5. LCMS calc. for C₂₅H₃₄N₅O₄ [M+H]⁺ m/z=468.3; Found 468.1.

### Step 3. 3-(1-Methyl-6-(2, 7-diazaspiro[3.5]nonan-7-yl)-1H-indazol-3-yl)piperidine-2,6-dione

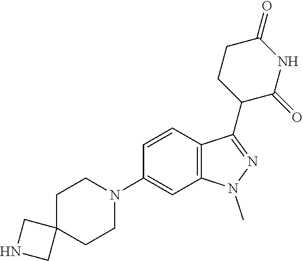 [SMILES: Cn1nc(C2CCC(=O)NC2=O)c2ccc(N3CCC4(CC3)CNC4)cc21]

The title compound was synthesized by procedures analogous to those outlined in Example 34, Step 3. LCMS calc. for C₂₀H₂₆N₅O₂ [M+H]⁺ m/z=368.2; Found 368.1.

### Step 4. N-(6-((1H-Pyrazol-1-yl)methyl)-4-methoxybenzo[d]isoxazol-3-yl)-3-bromo-2-methoxybenzenesulfonamide

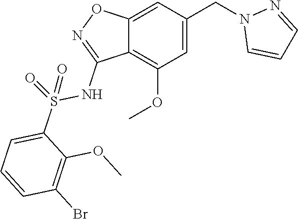 [SMILES: COc1c(Br)cccc1S(=O)(=O)Nc1noc2cc(Cn3cccn3)cc(OC)c12]

The title compound was synthesized by procedures analogous to those outlined in Example 12, Step 1, using 3-bromo-2-methoxybenzenesulfonyl chloride. LCMS calc. for C₁₉H₁₈BrN₄O₅S [M+H]⁺ m/z=493.0, 495.0; Found 493.0, 495.0.

### Step 5. N-(6-((1H-Pyrazol-1-yl)methyl)-4-methoxybenzo[d]isoxazol-3-yl)-3-(7-(3-(2,6-dioxopiperidin-3-yl)-1-methyl-1H-indazol-6-yl)-2,7-diazaspiro[3.5]nonan-2-yl)-2-methoxybenzenesulfonamide

To a solution N-(6-((1H-pyrazol-1-yl)methyl)-4-methoxybenzo[d]isoxazol-3-yl)-3-bromo-2-methoxybenzenesulfonamide (20 mg, 0.041 mmol) in DMF (1 mL) was added 3-(1-methyl-6-(2,7-diazaspiro[3.5]nonan-7-yl)-1H-indazol-3-yl)piperidine-2,6-dione (17.9 mg, 0.0487 mmol), cesium carbonate (52.8 mg, 0.162 mmol), and Pd-PEPPSI-iHeptCl (CAS: 1814936-54-3, 2.0 mg, 0.0020 mmol). The reaction was stirred for 17 h. The mixture was filtered and purified by prep-HPLC on a C18 column (15-60% CH₃CN/0.2% TFA (aq.)) to afford the title compound (10.4 mg, 0.0133 mmol, 32.9%) as a white solid. LCMS calc. for C₃₉H₄₂N₉O₇S [M+H]⁺ m/z=780.3; Found 780.5.

### Example A. HiBit KAT6A Degradation Assessment

The degradation activity of compounds was evaluated by measuring levels of KAT6A protein using the HiBiT system. These studies were conducted in HeLa cells expressing HiBit tagged KAT6A. Cells were maintained in a 37° C. incubator at 5% CO₂ in DMEM (ATCC, 30-2003) supplemented with 10% v/v FBS (Gibco, 26140-079) and 1% penicillin streptomycin. Cells were seeded in 384-well plates at a density of 2,000 cells/well. Compounds dissolved in DMSO were added in 9-point serial dilution. After 24 h of treatment, KAT6A levels were measured using the Nano-Glo® HiBiT Lytic Detection System (Promega catalog number N₃₀₃₀) per manufacturer's instructions. Luminescence signal was measured with a multimode plate reader (PHERAStar FSX, BMG Labtech). Luminescence values were normalized to background and DMSO controls to quantify KAT6A for each condition. Results were analyzed, and DC₅₀ and Dₘₐₓ values obtained using a four-parameter logistic curve fit. The results are summarized in Table 7.

TABLE 7
HiBiT KAT6A Degradation

| Ex. | DC₅₀ | Dₘₐₓ |
|---|---|---|
| 1 | B | A |
| 2 | B | A |
| 3 | B | B |
| 4 | C | B |
| 5 | A | A |
| 6 | A | A |
| 7 | A | A |
| 8 | A | B |
| 9 | A | A |
| 10 | B | A |
| 11 | A | A |
| 12 | B | A |
| 13 | C | B |
| 14 | A | A |
| 15 | A | B |
| 16 | A | A |
| 17 | A | A |
| 18 | B | B |
| 19 | C | B |
| 20 | A | A |
| 21 | A | A |
| 22 | A | A |
| 23 | A | A |
| 24 | A | A |
| 25 | A | A |
| 26 | B | A |
| 27 | B | B |
| 28 | A | A |
| 29 | C | A |
| 30 | A | B |
| 31 | A | A |
| 32 | A | A |
| 33 | A | A |
| 34 | B | A |
| 35 | B | A |
| 36 | A | A |
| 37 | C | B |
| 38 | A | A |
| 39 | A | A |
| 40 | B | B |
| 41 | A | B |
| 42 | B | A |
| 43 | B | B |
| 44 | C | B |
| 45 | C | C |
| 46 | A | A |
| 47 | C | B |
| 48 | B | A |
| 49 | C | C |
| 50 | B | A |
| 51 | A | B |
| 52 | A | B |
| 53 | A | B |
| 54 | B | A |
| 55 | A | A |
| 56 | A | A |
| 57 | A | B |
| 58 | A | A |
| 59 | A | A |
| 60 | A | A |
| 61 | A | A |
| 62 | A | A |
| 63 | A | A |
| 64 | A | B |
| 65 | B | A |
| 66 | A | A |
| 67 | B | B |
| 68 | B | A |
| 69 | A | B |
| 70 | A | A |
| 71 | B | B |
| 72 | A | B |
| 73 | A | A |
| 74 | B | A |
| 75 | A | A |
| 76 | C | C |
| 77 | A | A |
| 78 | B | B |
| 79 | B | B |
| 80 | B | A |
| 81 | A | A |
| 82 | A | A |
| 83 | A | A |
| 84 | A | A |
| 85 | A | A |
| 86 | B | B |
| 87 | A | A |
| 88 | B | B |
| 89 | A | A |
| 90 | A | A |
| 91 | A | B |
| 92 | A | B |
| 93 | A | A |
| 94 | A | B |
| 95 | A | B |
| 96 | A | B |
| 97 | A | A |
| 98 | B | B |
| 99 | A | B |
| 100 | B | B |
| 101 | A | B |
| 102 | B | A |
| 103 | A | A |
| 104 | A | B |
| 105 | B | A |
| 106 | B | B |
| 107 | B | B |
| 108 | A | A |
| 109 | A | B |
| 110 | — | C |
| 111 | — | C |
| 112 | A | A |
| 113 | A | A |
| 114 | A | B |
| 115 | A | A |
| 116 | A | A |
| 117 | A | A |
| 118 | A | A |
| 119 | B | B |
| 120 | — | C |

In Table 7, column DC₅₀, an “A” denotes DC₅₀ < 1 nM; a “B” denotes 1 nM ≤ DC₅₀ < 10 nM; a “C” denotes 10 nM ≤ DC₅₀ < 100 nM; a “D” denotes DC₅₀ > 100 nM. In Table 7, column Dₘₐₓ, an “A” denotes Dₘₐₓ > 75%; a “B” denotes 50% < Dₘₐₓ ≤ 75%; a “C” denotes 25% < Dₘₐₓ ≤ 50%; D represents that a Dₘₐₓ ≤ 25%.

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

1. A compound of Formula (I): 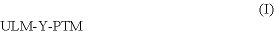
or a pharmaceutically acceptable salt or solvate thereof,
wherein Y-PTM is a moiety of Formula IA: 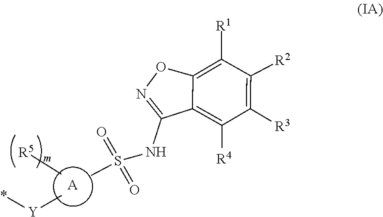
wherein
Y is a covalent bond, or chemical moiety that links PTM and ULM;
* is a point of attachment to ULM;
Ring A is a C₆-C₁₀ membered aryl group or a 5-10 membered heteroaryl group;
R¹ is H or 5-6 membered heteroaryl optionally substituted by methyl;
R² is H, C₁₋₄alk-O—C₁₋₄alkyl, or —(C(R⁸)₂)ₙ-(5-9 membered heteroaryl) optionally substituted by halogen, C₁-C₃ alkyl, —CH₂OH, or —OH;
R³ is H, halogen, C₁-C₄ alkyl, cyclopropyl, haloalkyl, C₁-C₄ alkoxy, or haloalkoxy;
R⁴ is H, halogen, C₁-C₄ alkyl, cyclopropyl, C₁-C₄ alkoxy, or —O-cyclopropyl;
each R⁵ is independently H, halogen, oxo, —OH, —CN, —NO₂, —C₁-C₆alkyl, —C₂-C₆alkenyl, —C₂-C₆alkynyl, C₀-C₁alk-aryl, C₀-C₁alk-heteroaryl, cycloalkyl, cycloalkenyl, heterocycloalkyl, or heterocycloalkenyl, —OR^{a}, —SR^{a}, —NR^{c}R^{d}, —NR^{a}R^{c}, —C(O)R^{b}, —OC(O)R^{a}, —C(O)OR^{a}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{d})(OR^{c}), or —S(O)₂R^{b};
each R^{a} is independently H, —C(O)R^{b}, —C(O)OR^{c}, —C(O)NR^{c}R^{d}, —C(═NR^{b})NR^{b}R^{c}, —C(═NOR^{b})NR^{b}R^{c}, —C(═NCN)NR^{b}R^{c}, —P(OR^{c})₂, —P(O)R^{c}R^{b}, —P(O)OR^{c}OR^{b}, —S(O)R^{b}, —S(O)NR^{c}R^{d}, —S(O)₂R^{b}, —S(O)₂NR^{c}R^{d}, SiR₃, —C₁-C₁₀alkyl, —C₁-C₁₀ haloalkyl, —C₂-C₁₀ alkenyl, —C₂-C₁₀ alkynyl, aryl, cycloalkyl, cycloalkenyl, heteroaryl, heterocycloalkyl, or heterocycloalkenyl;
each R^{b} is independently H, —C₁-C₆ alkyl, —C₁-C₆ haloalkyl, —C₂-C₆ alkenyl, —C₂-C₆ alkynyl, aryl, cycloalkyl, cycloalkenyl, heteroaryl, heterocycloalkyl, or heterocycloalkenyl;
each R^{c} or R^{d} is independently H, —C₁-C₁₀ alkyl, —C₂-C₆ alkenyl, —C₂-C₆ alkynyl, —OC₁-C₆alkyl, —O-cycloalkyl, aryl, heteroaryl, cycloalkyl, cycloalkenyl, heterocycloalkyl, or heterocycloalkenyl; or
R^{c} and R^{d}, together with the atom to which they are both attached, form a monocyclic or multicyclic heterocycloalkyl, or a monocyclic or multicyclic heterocyclo-alkenyl group;
each R⁸ is independently H, halogen, or C₁-C₄ alkyl;
m is 0, 1, 2, 3, or 4;
n is 0 or 1; and
ULM is 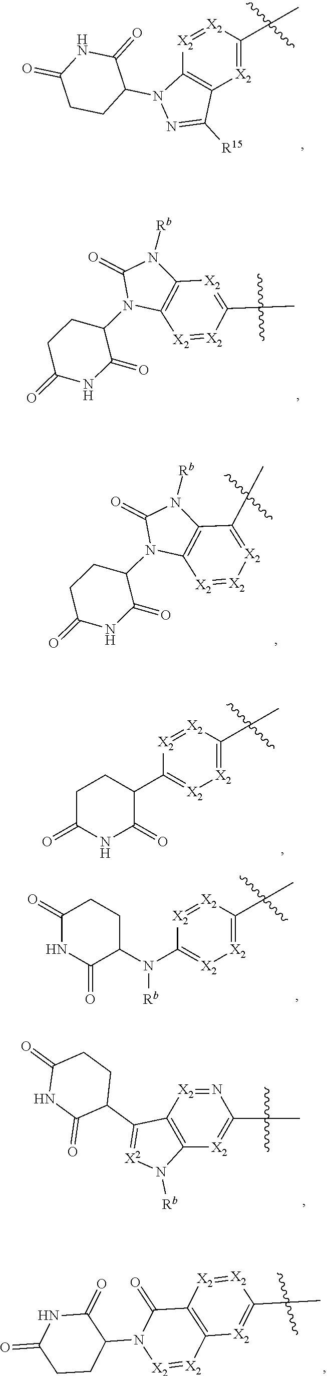 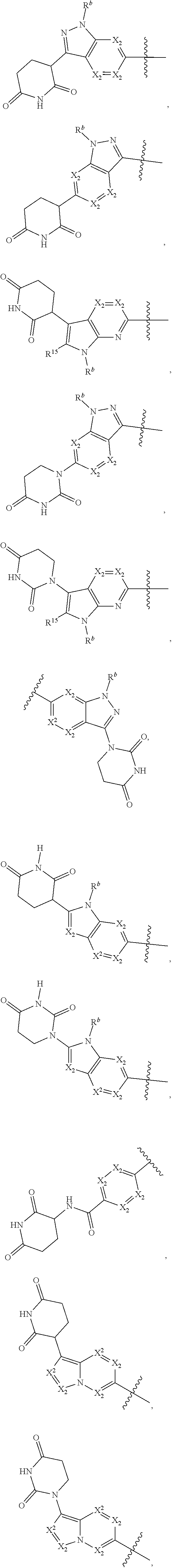 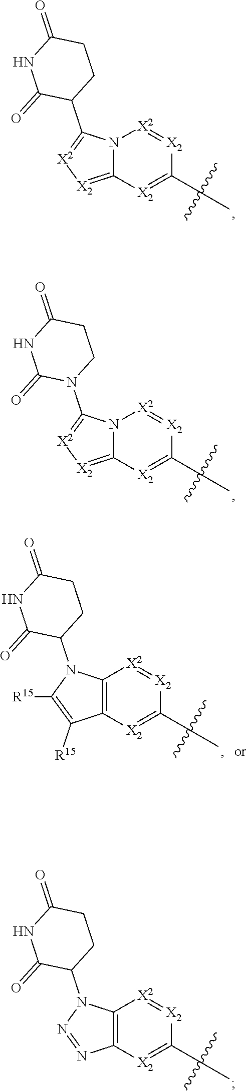
wherein [symbol]
is a point of attachment to Y;
each X₂ is independently N or CR¹⁵; and
each R¹⁵ is independently H, D, halogen, oxo, —OH, —CN, —NO₂, —C₁-C₆alkyl, —C₁-C₆ haloalkyl, —C₂-C₆alkenyl, —C₂-C₆alkynyl, C₀-C₁alk-aryl, C₀-C₁alk-heteroaryl, cycloalkyl, cycloalkenyl, heterocycloalkyl, or heterocycloalkenyl, —OR^{a}, —SR^{a}, —NR^{c}R^{d}, —NR^{a}R^{c}, —C(O)R^{b}, —OC(O)R^{a}, —C(O)OR^{a}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{d})(OR^{c}) or —S(O)₂R^{b};
wherein any of the —C₁-C₆alkyl, C₁-C₈ alkoxide, —C₂-C₆alkenyl, —C₂-C₆alkynyl, aryl, heteroaryl, cycloalkyl, cycloalkenyl, heterocycloalkyl, heterocycloalkenyl, spirocycloalkyl, spiroheterocycloalkyl and spiroheterocycloalkenyl groups in any of R₁—R₅, R₈, R₁₅, and ring Ais optionally substituted by 1-6 R^{f} groups; and
each R₁₅ independently D, oxo, halogen, C₁-C₈ alkoxy, C₁-C₈ alkyl, C₁₋₆ haloalkyl, C₁₋₆ haloalkoxy, —OH, —CN, —NO₂, —C₂₋₆ alkenyl, —C₂₋₆ alkynyl, C₆₋₁₀ aryl, C₅₋₁₂ heteroaryl, C₃₋₈ cycloalkyl, C₃₋₈ cycloalkenyl, C₃₋₈ heterocycloalkyl, C₃₋₈ heterocycloalkenyl, —OR^{a}, —SR^{a}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)R^{c}R^{d}, —P(O)(OR^{b})(OR^{b}), —B(OR^{c})(OR^{d}), —S(O)₂R^{b}, —C(O)NR^{b}OR^{b}, —S(O)₂OR^{b}, —OS(O)₂OR^{b}, or —OPO(OR^{b})(OR^{b}); wherein said C₁-C₈ alkyl is optionally substituted by 1-6 groups selected from D, halogen, —OH, —CN, —OR^{a}, —SR^{a}, —NR^{a}R^{d}, or NR^{c}R^{d}.

2.-4. (canceled)

5. The compound according to claim 1, wherein R² is —(C(R⁸)₂)ₙ-(5-9 membered heteroaryl) optionally substituted by halogen, C₁-C₃ alkyl, —CH₂OH, or —OH.

6. The compound according to claim 5, wherein R² is —(C(R⁸)₂)ₙ-(5-6 membered heteroaryl) optionally substituted by halogen, C₁-C₃ alkyl, —CH₂OH, or —OH.

7. The compound according to claim 6, wherein the 5-6 membered heteroaryl is a pyrazole, a pyrrole, a pyridine or a pyridazine.

8.-35. (canceled)

36. The compound according to claim 1, wherein Y is represented by the formula: 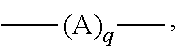
wherein:
q is an integer from 1 to 14;
each Ais independently selected from the group consisting of CR^{1a}R^{1b}, O, S, SO, SO₂, NR^{1c}, SO₂NR^{1c}, SONR^{1c}, SO(═NR^{1c}), SO(═NR^{1c})NR^{1d}, CONR^{1c}, NR^{1c}CONR^{1d}, NR^{1c}C(O)O, NR^{1c}SO₂NR^{1d}, CO, CR^{1a}═CR^{1b}, C≡C, SiR^{1a}R^{1b}, P(O)R^{1a}, P(O)OR^{1a}, (CR^{1a}R^{1b})₁₋₄, —(CR^{1a}R^{1b})₁₋₄O(CR^{1a}R^{1b})₁₋₄, —(CR^{1a}R^{1b})₁₋₄S(CR^{1a}R^{1b})₁₋₄, —(CR^{1a}R^{1b})₁₋₄NR^{1c}(CR^{1a}R^{1b})₁₋₄, NR^{1c}C(═NCN)NR^{1d}, NR^{1c}C(═NCN), NR^{1c}C(═CNO₂)NR^{1d}, 3-11 membered cycloalkyl, optionally substituted with 0-6 R^{1a} or R^{1b} groups, 3-11 membered heterocyclyl optionally substituted with 0-6 R^{1a} or R^{1b} groups, aryl optionally substituted with 0-6 R^{1a} or R^{1b} groups, or heteroaryl optionally substituted with 0-6 R^{1a} or R^{1b} groups,
wherein R^{1a}, R^{1b}, R^{1c}, R^{1d} and R^{1e} are each independently, —H, -halo, —C₁-C₈alkyl, —O—C₁-C₈alkyl, —C₁-C₆haloalkyl, —S—C₁-C₈alkyl, —NHC₁-C₈alkyl, —N(C₁-C₈alkyl)₂, 3-11 membered cycloalkyl, aryl, heteroaryl, 3-11 membered heterocyclyl, —O-(3-11 membered cycloalkyl), —S-(3-11 membered cycloalkyl), NH-(3-11 membered cycloalkyl), N(3-11 membered cycloalkyl)₂, N-(3-11 membered cycloalkyl)(C₁-C₈alkyl), —OH, —NH₂, —SO₂C₁-C₈alkyl, —SO₂-aryl, —SO₂-heteroaryl, SO(NH)C₁-C₈alkyl, P(O)(OC₁-C₈alkyl)(C₁-C₈alkyl), —P(O)(OC₁-C₈alkyl)₂, —C≡C—C₁-C₈alkyl, —C≡CH, —CH═CH(C₁-C₈alkyl), —C(C₁-C₈alkyl)═CH(C₁-C₈alkyl), —C(C₁-C₈alkyl)═C(C₁-C₈alkyl)₂, —Si(OH)₃, —Si(C₁-C₈alkyl)₃, —Si(OH)(C₁-C₈alkyl)₂, —C(O)C₁-C₈alkyl, —C(O)OC₁-C₈alkyl, —CO₂H, —CN, —CF₃, —CHF₂, —CH₂F, —NO₂, —SF₅, —SO₂NHC₁-C₈alkyl, —SO₂N(C₁-C₈alkyl)₂, —SO(NH)NHC₁-C₈alkyl, —SO(NH)N(C₁-C₈alkyl)₂, —SONHC₁-C₈alkyl, —SON(C₁-C₈alkyl)₂, —CONHC₁-C₈alkyl, —CON(C₁-C₈alkyl)₂, —N(C₁-C₈alkyl)CONH(C₁-C₈alkyl), —N(C₁-C₈alkyl)CON(C₁-C₈alkyl)₂, —NHCONH(C₁-C₈alkyl), —NHCON(C₁-C₈alkyl)₂, —NHCONH₂, —N(C₁-C₈alkyl)SO₂NH(C₁-C₈alkyl), —N(C₁-C₈alkyl)SO₂N(C₁-C₈alkyl)₂, —NHSO₂NH(C₁-C₈alkyl), —NHSO₂N(C₁-C₈alkyl)₂, or —NHSO₂NH₂; and where R^{1a} or R^{1b}, each independently may be optionally linked to other groups to form cycloalkyl and/or heterocyclyl moiety, optionally substituted with 0-4 R^{1e} groups.

37. (canceled)

38. The compound according to claim 1, wherein Y is 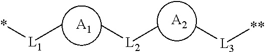
wherein
** is a point of attachment to PTM;
L₁, L₂, and L₃ are each independently a bond, CR^{1a}R^{1b}, O, SO, SO₂, NR^{c}SO₂NR^{1c}, SONR^{1c}, SO(═NR^{1c}), SO(═NR^{1c})NR^{1d}, CONR^{1c}, NR^{1c}CONR^{1d}, NR^{1c}C(O)O, NR^{1c}SO₂NR^{1d}, CO, CR^{1a}═CR^{1b}, C≡C, SiR^{1a}R^{1b}, P(O)R^{1a}, P(O)OR^{1a}, (CR^{1a}R^{1b})₁₋₄, —(CR^{1a}R^{1b})₁₋₄O(CR^{1a}R^{1b})₁₋₄, —(CR^{1a}R^{1b})₁₋₄S(CR^{1a}R^{1b})₁₋₄, —(CR^{1a}R^{1b})₁₋₄NR^{c}(CR^{1a}R^{b})₁₋₄, NR^{1c}C(═NCN)NR^{1d}, NR^{1c}C(═NCN), NR^{1c}C(═CNO₂)NR^{1d};
ring A₁ and ring A₂ are each independently 3-12 membered cycloalkyl, optionally substituted with 1-6 R^{1a} or R^{1b} groups; 3-12 membered heterocyclyl optionally substituted with 1-6 R^{1a} or R^{1b} groups; aryl optionally substituted with 1-6 R^{1a} or R^{1b} groups; or heteroaryl optionally substituted with 1-6 R^{1a} or R^{1b} groups;
wherein R^{1a}, R^{1b}, R^{1c}, R^{1d} and R^{1e} are each independently, —H, -halo, —C₁-C₈alkyl, —O—C₁-C₈alkyl, —C₁-C₆haloalkyl, —S—C₁-C₈alkyl, —NHC₁-C₈alkyl, —N(C₁-C₈alkyl)₂, 3-11 membered cycloalkyl, aryl, heteroaryl, 3-11 membered heterocyclyl, —O-(3-11 membered cycloalkyl), —S-(3-11 membered cycloalkyl), NH-(3-11 membered cycloalkyl), N(3-11 membered cycloalkyl)₂, N-(3-11 membered cycloalkyl)(C₁-C₈alkyl), —OH, —NH₂, —SH, —SO₂C₁-C₈alkyl, —SO₂-aryl, —SO₂-heteroaryl, SO(NH)C₁-C₈alkyl, P(O)(OC₁-C₈alkyl)(C₁-C₈alkyl), —P(O)(OC₁-C₈alkyl)₂, —C≡C—C₁-C₈alkyl, —C≡CH, —CH═CH(C₁-C₈alkyl), —C(C₁-C₈alkyl)═CH(C₁-C₈alkyl), —C(C₁-C₈alkyl)═C(C₁-C₈alkyl)₂, —Si(OH)₃, —Si(C₁-C₈alkyl)₃, —Si(OH)(C₁-C₈alkyl)₂, —C(O)C₁-C₈alkyl, —C(O)OC₁-C₈alkyl, —CO₂H, —CN, —CF₃, —CHF₂, —CH₂F, —NO₂, —SF₅, —SO₂NHC₁-C₈alkyl, —SO₂N(C₁-C₈alkyl)₂, —SO(NH)NHC₁-C₈alkyl, —SO(NH)N(C₁-C₈alkyl)₂, —SONHC₁-C₈alkyl, —SON(C₁-C₈alkyl)₂, —CONHC₁-C₈alkyl, —CON(C₁-C₈alkyl)₂, —N(C₁-C₈alkyl)CONH(C₁-C₈alkyl), —N(C₁-C₈alkyl)CON(C₁-C₈alkyl)₂, —NHCONH(C₁-C₈alkyl), —NHCON(C₁-C₈alkyl)₂, —NHCONH₂, —N(C₁-C₈alkyl)SO₂NH(C₁-C₈alkyl), —N(C₁-C₈alkyl)SO₂N(C₁-C₈alkyl)₂, —NHSO₂NH(C₁-C₈alkyl), —NHSO₂N(C₁-C₈alkyl)₂, or —NHSO₂NH₂; and where R^{1a} or R^{1b}, each independently may be optionally linked to other groups to form cycloalkyl and/or heterocyclyl moiety, optionally substituted with 0-4 R^{1e} groups.

39. The compound according to claim 1, wherein Y is 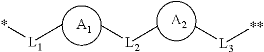
wherein
* is a point of attachment to ULM
** is a point of attachment to PTM;
L₁ is a bond, (C(R¹⁰)₂)ₚ, or CO;
L₂ is a bond, (C(R¹⁰)₂)ₚ, or CO;
L₃ is a bond, (C(R¹⁰)₂)ₚ, or CO;
p is 1, 2, 3 or 4;
each R¹⁰ is independently H or C₁-C₄ alkyl;
ring A₁ is a 3-7 membered cycloalkyl group, a 4-10-membered heterocycloalkyl group, an aryl group, or a heteroaryl group; and
ring A₂ is a 3-7 membered cycloalkyl group, a 4-12-membered heterocycloalkyl group, an aryl group, or a heteroaryl group;
wherein ring A₁ and ring A₂ are optionally substituted with 1-6 R^{1a} or R^{1b} groups.

40.-49. (canceled)

50. The compound according to claim 39, wherein Y is 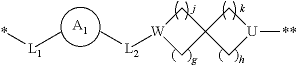
wherein
* is a point of attachment to ULM;
** is a point of attachment to PTM;
W is CR₁₁ or N;
U is CR₁₁ or N;
R₁₁ is H, fluoro, C₁₋₆alkyl, or C₁₋₆alkoxide; and
each g, h, j and k is independently 0, 1 or 2.

51.-57. (canceled)

58. The compound according to claim 39, wherein Y is 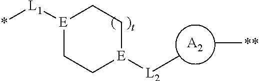
wherein
* is a point of attachment to ULM;
** is a point of attachment to PTM;
each E is independently CR₁₁ or N;
R₁₁ is H, F, C₁₋₆alkyl, or C₁₋₆alkoxide; and
t is 0, 1, 2 or 3.

59.-64. (canceled)

65. The compound according to claim 36, wherein Y is 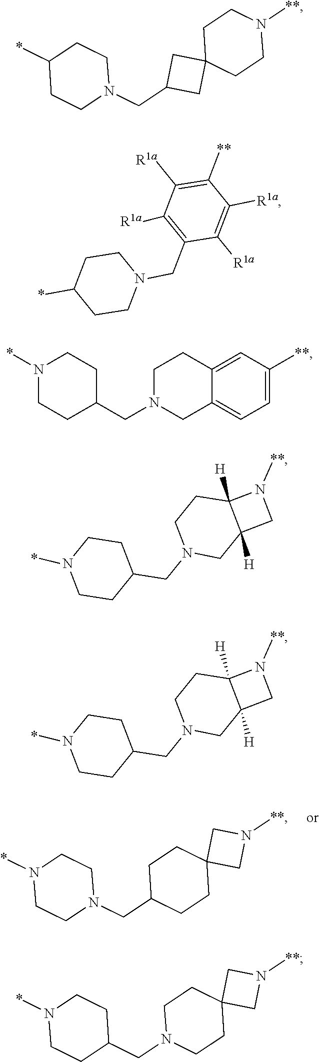
wherein * is a point of attachment to ULM and ** is a point of attachment to PTM.

66.-72. (canceled)

73. The compound according to claim 1, wherein Ring A is 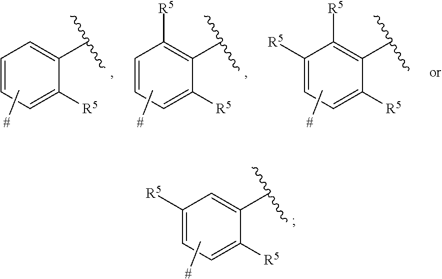
wherein [symbol]
is a point of attachment to the sulfonamide group and # is a point of attachment to Y.

74. The compound according to claim 73, wherein Ring A is 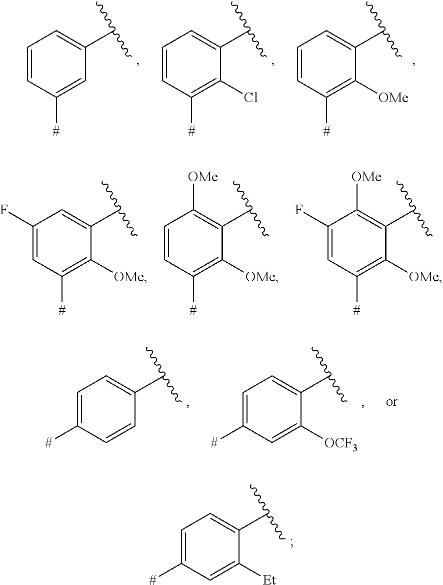
wherein [symbol]
is a point of attachment to the sulfonamide group and # is a point of attachment to Y.

75.-78. (canceled)

79. The compound according to claim 1, or a pharmaceutically acceptable salt thereof, wherein Y-PTM has the formula IA-1:

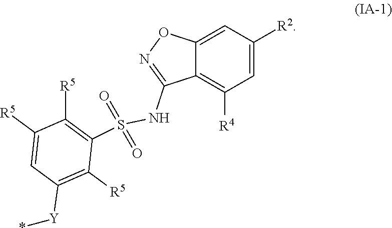

80. The compound according to claim 1, or a pharmaceutically acceptable salt thereof, wherein Y-PTM has the formula IA-2: 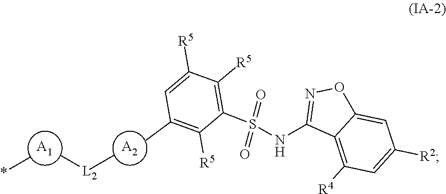
wherein
* is a point of connection to ULM;
L₂ is a bond or (C(R¹⁰)₂)ₚ;
p is 1, 2, 3 or 4;
each R¹⁰ is independently H or C₁-C₄ alkyl;
ring A₁ is a 3-7 membered cycloalkyl group, a 4-10-membered heterocycloalkyl group, an aryl group, or a heteroaryl group; and
ring A₂ is a 3-7 membered cycloalkyl group, a 4-12-membered heterocycloalkyl group, an aryl group, or a heteroaryl group.

81. The compound according to claim 1, or a pharmaceutically acceptable salt thereof, wherein Y-PTM has the formula IA-3: 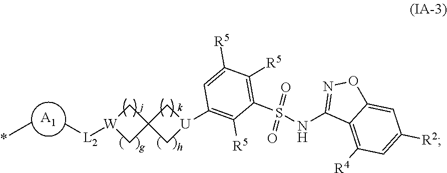
wherein
* is a point of connection to ULM;
L₂ is a bond or (C(R¹⁰)₂)ₚ;
p is 1, 2, 3 or 4;
each R¹⁰ is independently H or C₁-C₄ alkyl;
ring A₁ is a 3-7 membered cycloalkyl group, a 4-10-membered heterocycloalkyl group, an aryl group, or a heteroaryl group;
W is CR₁₁ or N;
U is CR₁₁ or N;
R₁₁ is H, F, C₁₋₆alkyl, or C₁₋₆alkoxide; and
each g, h, j and k is independently 0, 1, 2 or 3.

82.-88. (canceled)

89. The compound according to claim 1, or a pharmaceutically acceptable salt thereof, wherein Y-PTM has formula IA-4 or formula IA-5: 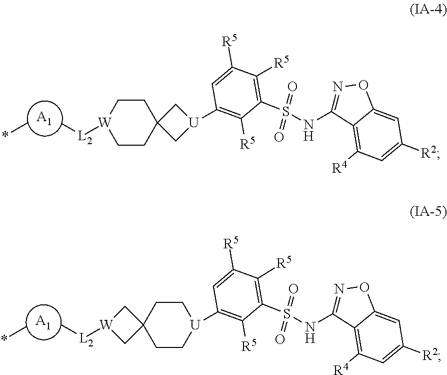
wherein
* is a point of connection to ULM;
L₂ is a bond or (C(R¹⁰)₂)ₚ;
p is 1, 2, 3 or 4;
each R¹⁰ is independently H or C₁-C₄ alkyl;
ring A₁ is a 3-7 membered cycloalkyl group, a 4-10-membered heterocycloalkyl group, an aryl group, or a heteroaryl group;
W is CR₁₁ or N;
U is CR₁₁ or N; and
R₁₁ is H, F, C₁₋₆alkyl, or C₁₋₆alkoxide.

90. The compound of claim 38, wherein ring A₁ is: 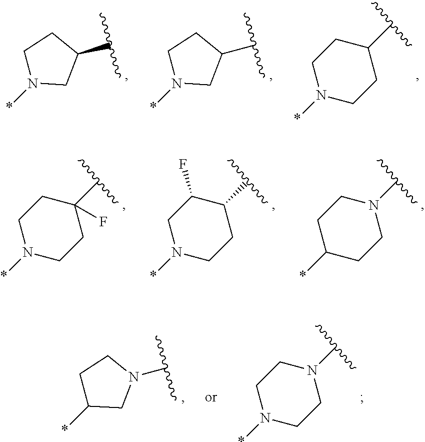
wherein [symbol]
is a point of attachment to L₂ and * is a point of attachment to ULM.

91. The compound according to claim 89, or a pharmaceutically acceptable salt thereof, wherein Y-PTM has formula IA-6 or formula IA-7: 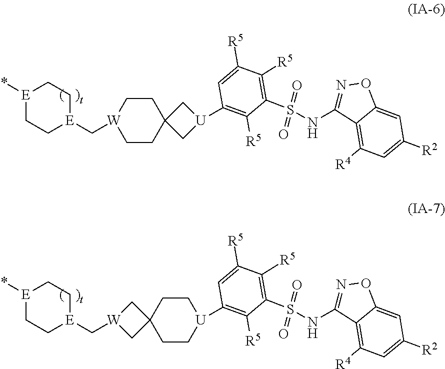
wherein
* is a point of connection to ULM;
W is CR₁₁ or N;
U is CR₁₁ or N; and
R₁₁ is H, F, C₁₋₆alkyl, or C₁₋₆alkoxide;
each E is independently CR₁₁ or N; and
t is 0, 1, 2 or 3.

92. The compound according to claim 1, or a pharmaceutically acceptable salt thereof, wherein Y-PTM has formula IA-8: 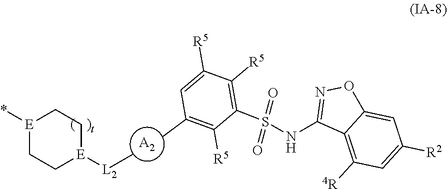
wherein
* is a point of connection to ULM;
L₂ is a bond or (C(R¹⁰)₂)ₚ;
p is 1, 2, 3 or 4;
each R¹⁰ is independently H or C₁-C₄ alkyl;
ring A₂ is a 3-7 membered cycloalkyl group, a 4-10-membered heterocycloalkyl group, an aryl group, or a heteroaryl group;
each E is independently CR₁₁ or N;
R₁₁ is H, F, C₁₋₆alkyl, or C₁₋₆alkoxide; and
t is 0, 1, 2 or 3.

93.-96. (canceled)

97. The compound according to claim 91, or a pharmaceutically acceptable salt thereof, wherein Y-PTM has formula IA-9: 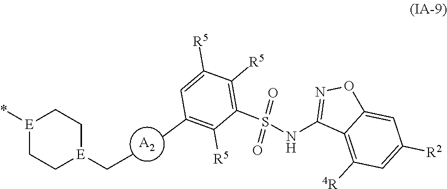
wherein
* is a point of connection to ULM;
L₂ is a bond or (C(R¹⁰)₂)ₚ;
p is 1, 2, 3 or 4;
each R¹⁰ is independently H or C₁-C₄ alkyl;
ring A₂ is a 3-7 membered cycloalkyl group, a 4-10-membered heterocycloalkyl group, an aryl group, or a heteroaryl group;
each E is independently CR₁₁ or N; and
R₁₁ is H, F, C₁₋₆alkyl, or C₁₋₆alkoxide.

98. The compound of claim 38, wherein ring A₂ is selected from: 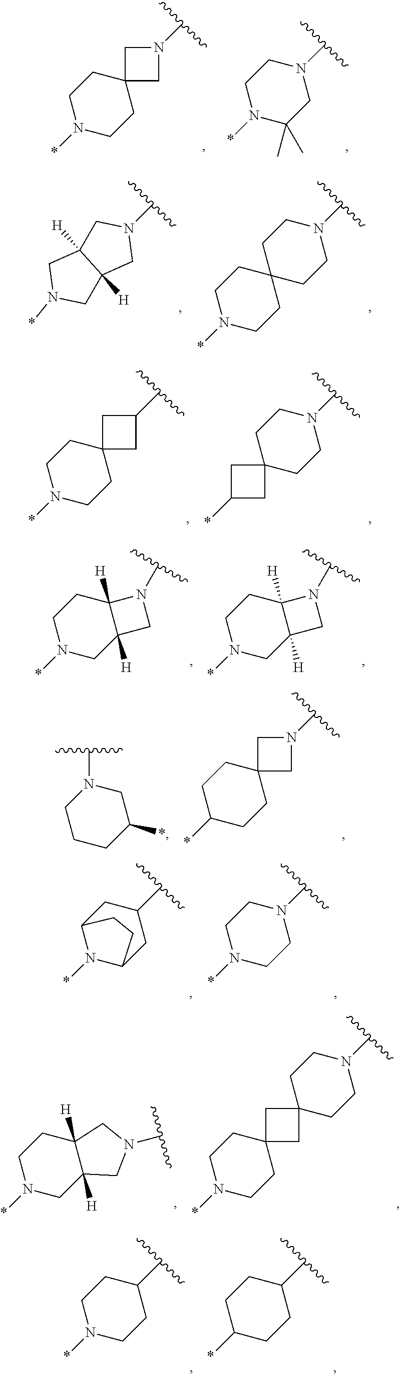 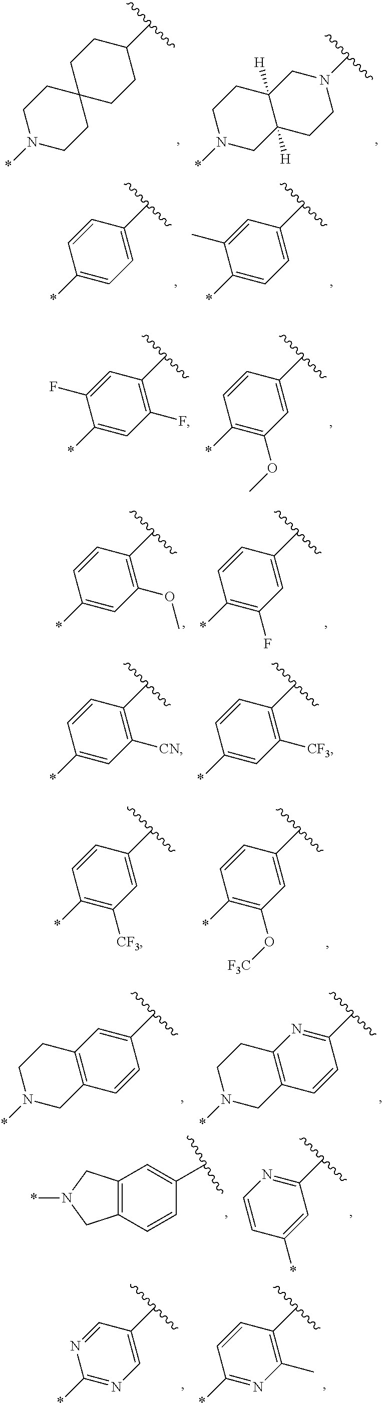 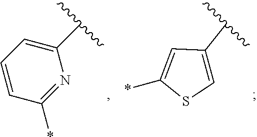 [SMILES: *c1cccc(*)n1 ; *c1csc(*)c1]
wherein [symbol]
is a point of attachment to PLM and * is a point of attachment to L₂.

99.-103. (canceled)

104. The compound according to claim 79, wherein R² is —(C(R⁸)₂)ₙ-(5-9 membered heteroaryl).

105. The compound according to claim 79, wherein R² is N

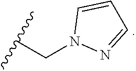 [SMILES: *Cn1cccn1]

106. The compound according to claim 1, wherein ULM is

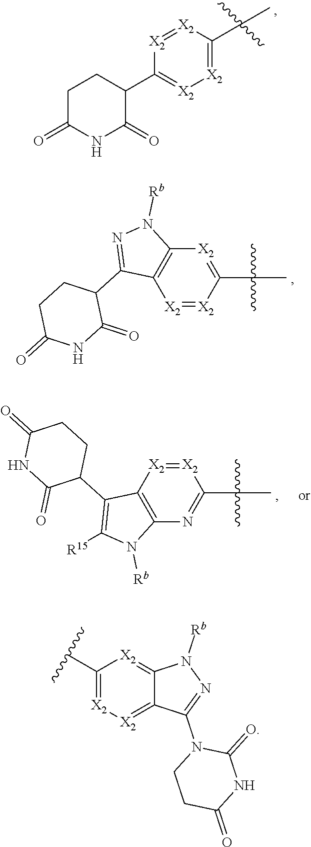

107. (canceled)

108. (canceled)

109. The compound according to claim 1, wherein ULM is 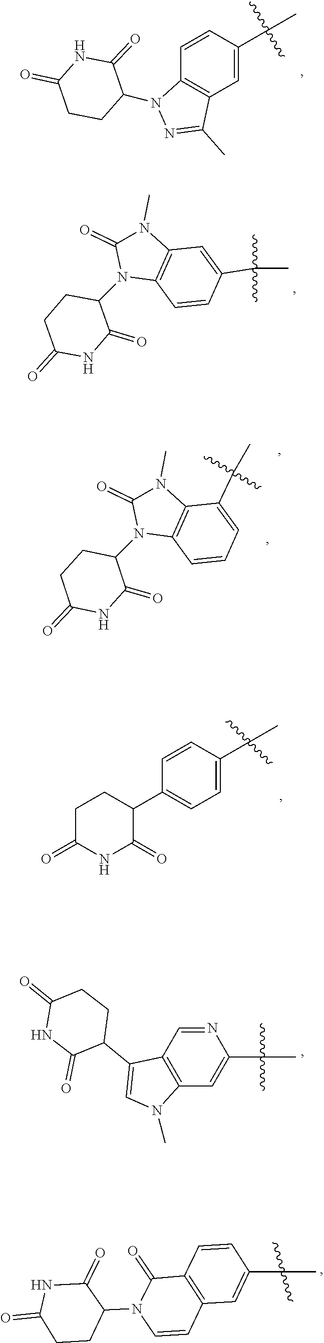 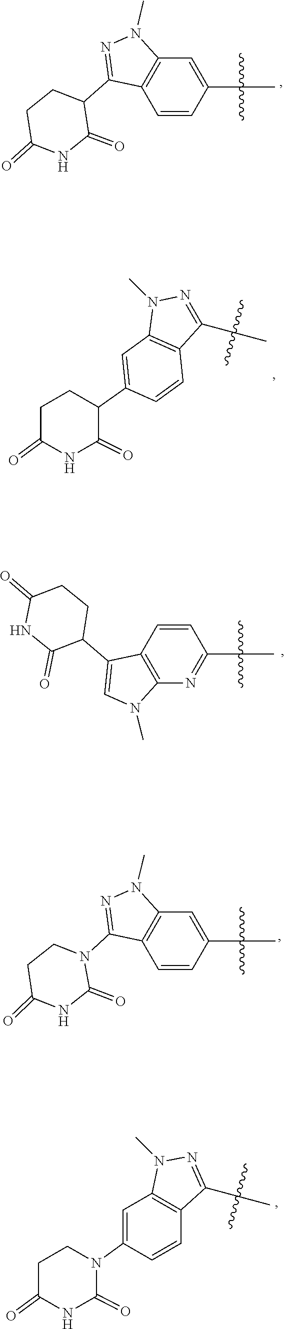 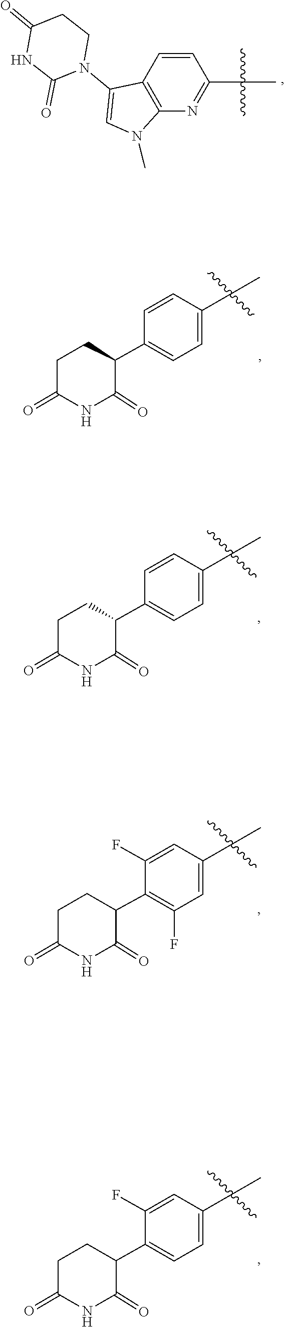 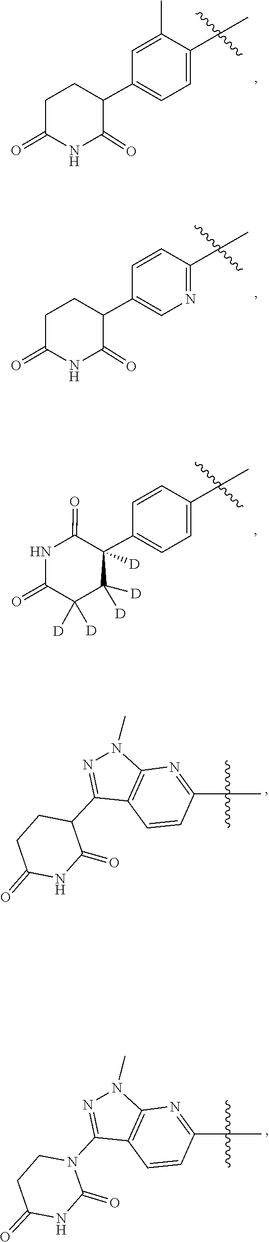 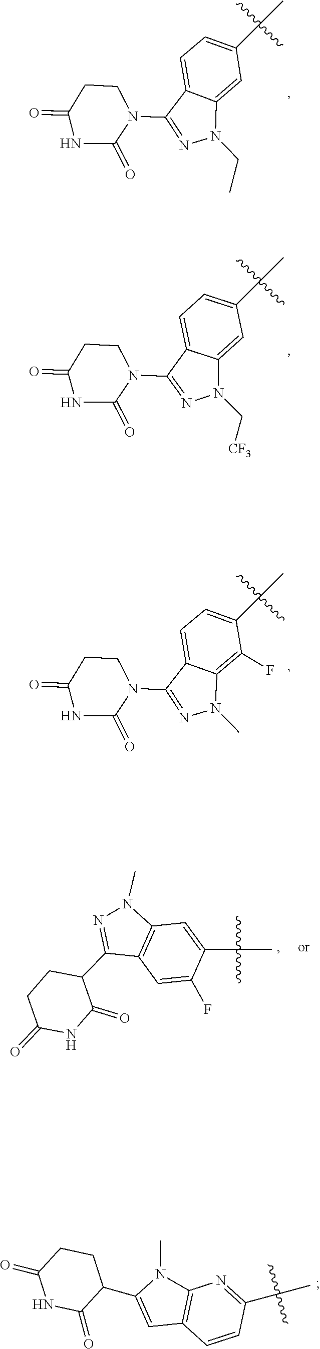
wherein [symbol] is a point of attachment to Y.

110. The compound according to claim 1, wherein ULM is 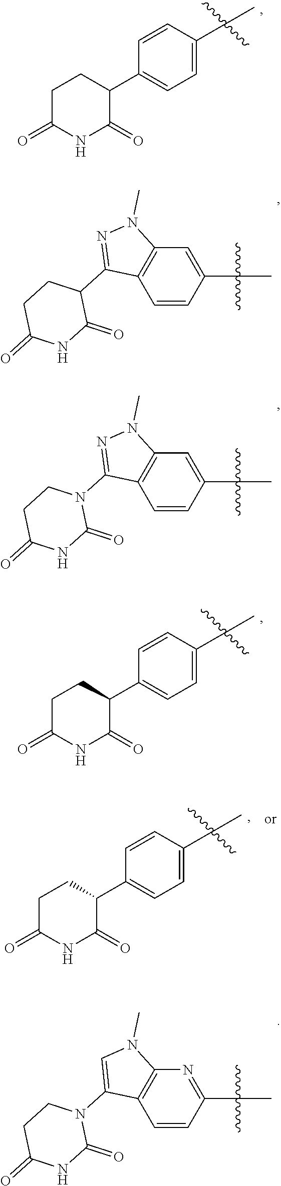
wherein [symbol] is a point of attachment to Y.

111.-116. (canceled)

117. The compound according to claim 1 that is:
N-(6-((1H-Pyrazol-1-yl)methyl)-4-methoxybenzo[d]isoxazol-3-yl)-3-(7-(((3S)-1-(1-(2,6-dioxopiperidin-3-yl)-3-methyl-1H-indazol-5-yl)pyrrolidin-3-yl)methyl)-2,7-diazaspiro[3.5]nonan-2-yl)benzenesulfonamide;
N-(6-((1H-Pyrazol-1-yl)methyl)-4-methoxybenzo[d]isoxazol-3-yl)-3-(7-((1-(1-(2,6-dioxopiperidin-3-yl)-3-methyl-2-oxo-2,3-dihydro-1H-benzo[d]imidazol-5-yl)piperidin-4-yl)methyl)-2,7-diazaspiro[3.5]nonan-2-yl)benzenesulfonamide;
N-(6-((1H-Pyrazol-1-yl)methyl)-4-methoxybenzo[d]isoxazol-3-yl)-3-(7-((1-(4-(2,6-dioxopiperidin-3-yl)phenyl)pyrrolidin-3-yl)methyl)-2,7-diazaspiro[3.5]nonan-2-yl)benzenesulfonamide;
N-(6-((1H-Pyrazol-1-yl)methyl)-4-methoxybenzo[d]isoxazol-3-yl)-3-(7-((1-(3-(2,6-dioxopiperidin-3-yl)-1-methyl-1H-pyrrolo[3,2-c]pyridin-6-yl)piperidin-4-yl)methyl)-2,7-diazaspiro[3.5]nonan-2-yl)benzenesulfonamide;
N-(6-((1H-Pyrazol-1-yl)methyl)-4-methoxybenzo[d]isoxazol-3-yl)-3-(7-((1-(2-(2,6-dioxopiperidin-3-yl)-1-oxo-1,2-dihydroisoquinolin-6-yl)piperidin-4-yl)methyl)-2,7-diazaspiro[3.5]nonan-2-yl)-2-methoxybenzenesulfonamide;
N-(6-((1H-Pyrazol-1-yl)methyl)-4-methoxybenzo[d]isoxazol-3-yl)-3-(7-((1-(1-(2,6-dioxopiperidin-3-yl)-3-methyl-1H-indazol-5-yl)piperidin-4-yl)methyl)-2,7-diazaspiro[3.5]nonan-2-yl)-2-methoxybenzenesulfonamide;
N-(6-((1H-Pyrazol-1-yl)methyl)-4-methoxybenzo[d]isoxazol-3-yl)-3-(7-((1-(3-(2,6-dioxopiperidin-3-yl)-1-methyl-1H-indazol-6-yl)piperidin-4-yl)methyl)-2,7-diazaspiro[3.5]nonan-2-yl)-2-methoxybenzenesulfonamide;
N-(6-((1H-Pyrazol-1-yl)methyl)-4-methoxybenzo[d]isoxazol-3-yl)-3-(7-((1-(6-(2,6-dioxopiperidin-3-yl)-1-methyl-1H-indazol-3-yl)piperidin-4-yl)methyl)-2,7-diazaspiro[3.5]nonan-2-yl)-2-methoxybenzenesulfonamide;
N-(6-((1H-Pyrazol-1-yl)methyl)-4-methoxybenzo[d]isoxazol-3-yl)-3-(7-((1-(3-(2,6-dioxopiperidin-3-yl)-1-methyl-1H-pyrrolo[2,3-b]pyridin-6-yl)piperidin-4-yl)methyl)-2,7-diazaspiro[3.5]nonan-2-yl)-2-methoxybenzenesulfonamide;
N-(6-((1H-Pyrazol-1-yl)methyl)-4-methoxybenzo[d]isoxazol-3-yl)-3-(7-((1-(3-(2,4-dioxotetrahydropyrimidin-1(2H)-yl)-1-methyl-1H-indazol-6-yl)piperidin-4-yl)methyl)-2,7-diazaspiro[3.5]nonan-2-yl)-2,4-dimethoxybenzenesulfonamide;
N-(6-((1H-Pyrazol-1-yl)methyl)-4-methoxybenzo[d]isoxazol-3-yl)-3-(7-((1-(3-(2,6-dioxopiperidin-3-yl)-1-methyl-1H-indazol-6-yl)piperidin-4-yl)methyl)-2,7-diazaspiro[3.5]nonan-2-yl)-2,6-dimethoxybenzenesulfonamide;
N-(6-((1H-Pyrazol-1-yl)methyl)-4-methoxybenzo[d]isoxazol-3-yl)-4-(7-((1-(1-(2,6-dioxopiperidin-3-yl)-3-methyl-1H-indazol-5-yl)piperidin-4-yl)methyl)-2,7-diazaspiro[3.5]nonan-2-yl)benzenesulfonamide;
N-(6-((1H-Pyrazol-1-yl)methyl)-4-methoxybenzo[d]isoxazol-3-yl)-4-(7-((1-(2-(2,6-dioxopiperidin-3-yl)-1-oxo-1,2-dihydroisoquinolin-6-yl)piperidin-4-yl)methyl)-2,7-diazaspiro[3.5]nonan-2-yl)benzenesulfonamide;
N-(6-((1H-Pyrazol-1-yl)methyl)-4-methoxybenzo[d]isoxazol-3-yl)-4-(7-((1-(1-(2,6-dioxopiperidin-3-yl)-3-methyl-1H-indazol-5-yl)piperidin-4-yl)methyl)-2,7-diazaspiro[3.5]nonan-2-yl)-2-methoxybenzenesulfonamide;
N-(6-((1H-Pyrazol-1-yl)methyl)-4-methoxybenzo[d]isoxazol-3-yl)-4-(7-((1-(1-(2,6-dioxopiperidin-3-yl)-3-methyl-2-oxo-2,3-dihydro-1H-benzo[d]imidazol-5-yl)piperidin-4-yl)methyl)-2,7-diazaspiro[3.5]nonan-2-yl)-2-methoxybenzenesulfonamide;
N-(6-((1H-Pyrazol-1-yl)methyl)-4-methoxybenzo[d]isoxazol-3-yl)-4-(4-((1-(1-(2,6-dioxopiperidin-3-yl)-3-methyl-2-oxo-2,3-dihydro-1H-benzo[d]imidazol-5-yl)piperidin-4-yl)methyl)-3,3-dimethylpiperazin-1-yl)benzenesulfonamide;
N-(6-((1H-Pyrazol-1-yl)methyl)-4-methoxybenzo[d]isoxazol-3-yl)-4-((3aS,6aS)-5-((1-(1-(2,6-dioxopiperidin-3-yl)-3-methyl-2-oxo-2,3-dihydro-1H-benzo[d]imidazol-5-yl)piperidin-4-yl)methyl)hexahydropyrrolo[3,4-c]pyrrol-2(1H)-yl)benzenesulfonamide;
N-(6-((1H-Pyrazol-1-yl)methyl)-4-methoxybenzo[d]isoxazol-3-yl)-4-((3aR,6aS)-5-((1-(1-(2,6-dioxopiperidin-3-yl)-3-methyl-2-oxo-2,3-dihydro-1H-benzo[d]imidazol-5-yl)piperidin-4-yl)methyl)hexahydropyrrolo[3,4-c]pyrrol-2(1H)-yl)-2-methoxybenzenesulfonamide;
N-(6-((1H-Pyrazol-1-yl)methyl)-4-methoxybenzo[d]isoxazol-3-yl)-3-(9-((1-(4-(2,6-dioxopiperidin-3-yl)phenyl)piperidin-4-yl)methyl)-3,9-diazaspiro[5.5]undecan-3-yl)benzenesulfonamide;
N-(6-((1H-Pyrazol-1-yl)methyl)-4-methoxybenzo[d]isoxazol-3-yl)-3-(9-((1-(3-(2,6-dioxopiperidin-3-yl)-1-methyl-1H-indazol-6-yl)piperidin-4-yl)methyl)-3, 9-diazaspiro[5.5]undecan-3-yl)benzenesulfonamide;
N-(6-((1H-Pyrazol-1-yl)methyl)-4-methoxybenzo[d]isoxazol-3-yl)-3-(7-((1-(3-(2,6-dioxopiperidin-3-yl)-1-methyl-1H-indazol-6-yl)piperidin-4-yl)methyl)-2,7-diazaspiro[3.5]nonan-2-yl)-2-(2,2,2-trifluoroethoxy)benzenesulfonamide;
N-(6-((1H-Pyrazol-1-yl)methyl)-4-methoxybenzo[d]isoxazol-3-yl)-2-chloro-3-(7-((1-(3-(2,6-dioxopiperidin-3-yl)-1-methyl-1H-indazol-6-yl)piperidin-4-yl)methyl)-2,7-diazaspiro[3.5]nonan-2-yl)benzenesulfonamide;
N-(6-((1H-Pyrazol-1-yl)methyl)-4-methoxybenzo[d]isoxazol-3-yl)-3-(7-((1-(3-(2,6-dioxopiperidin-3-yl)-1-methyl-1H-indazol-6-yl)piperidin-4-yl)methyl)-2,7-diazaspiro[3.5]nonan-2-yl)-5-fluoro-2-methoxybenzenesulfonamide;
N-(6-((1H-Pyrazol-1-yl)methyl)-4-methoxybenzo[d]isoxazol-3-yl)-5-chloro-3-(7-((1-(3-(2,6-dioxopiperidin-3-yl)-1-methyl-1H-indazol-6-yl)piperidin-4-yl)methyl)-2,7-diazaspiro[3.5]nonan-2-yl)-2-methoxybenzenesulfonamide;
N-(6-((1H-Pyrazol-1-yl)methyl)-4-methoxybenzo[d]isoxazol-3-yl)-3-(7-((1-(3-(2,6-dioxopiperidin-3-yl)-1-methyl-1H-indazol-6-yl)piperidin-4-yl)methyl)-2,7-diazaspiro[3.5]nonan-2-yl)-2-methoxy-5-methylbenzenesulfonamide;
N-(6-((1H-pyrazol-1-yl)methyl)-4-methoxybenzo[d]isoxazol-3-yl)-3-(7-((1-(3-(2,6-dioxopiperidin-3-yl)-1-methyl-1H-indazol-6-yl)piperidin-4-yl)methyl)-2,7-diazaspiro[3.5]nonan-2-yl)-2-(trifluoromethyl)benzenesulfonamide;
N-(6-((1H-pyrazol-1-yl)methyl)-4-methoxybenzo[d]isoxazol-3-yl)-3-(7-((1-(3-(2,6-dioxopiperidin-3-yl)-1-methyl-1H-indazol-6-yl)piperidin-4-yl)methyl)-2,7-diazaspiro[3.5]nonan-2-yl)-2-fluorobenzenesulfonamide;
N-(6-((1H-Pyrazol-1-yl)methyl)-4-methoxybenzo[d]isoxazol-3-yl)-3-(7-((1-(3-(2,6-dioxopiperidin-3-yl)-1-methyl-1H-indazol-6-yl)piperidin-4-yl)methyl)-2,7-diazaspiro[3.5]nonan-2-yl)-5-fluoro-2,6-dimethoxybenzenesulfonamide;
N-(6-((1H-Pyrazol-1-yl)methyl)-4-methoxybenzo[d]isoxazol-3-yl)-5-(7-((1-(4-(2,6-dioxopiperidin-3-yl)phenyl)piperidin-4-yl)methyl)-2,7-diazaspiro[3.5]nonan-2-yl)-2-methylbenzenesulfonamide;
N-(6-((1H-Pyrazol-1-yl)methyl)-4-methoxybenzo[d]isoxazol-3-yl)-3-(7-((1-(4-(2,6-dioxopiperidin-3-yl)phenyl)piperidin-4-yl)methyl)-2,7-diazaspiro[3.5]nonan-2-yl)-2-methylbenzenesulfonamide;
N-(6-((1H-Pyrazol-1-yl)methyl)-4-methoxybenzo[d]isoxazol-3-yl)-4-(7-((1-(3-(2,6-dioxopiperidin-3-yl)-1-methyl-1H-indazol-6-yl)piperidin-4-yl)methyl)-2,7-diazaspiro[3.5]nonan-2-yl)-2-methylbenzenesulfonamide;
N-(6-((1H-Pyrazol-1-yl)methyl)-4-methoxybenzo[d]isoxazol-3-yl)-4-(7-((1-(3-(2,6-dioxopiperidin-3-yl)-1-methyl-1H-indazol-6-yl)piperidin-4-yl)methyl)-2,7-diazaspiro[3.5]nonan-2-yl)-2-ethylbenzenesulfonamide;
N-(6-((1H-Pyrazol-1-yl)methyl)-4-methoxybenzo[d]isoxazol-3-yl)-4-(7-((1-(3-(2,6-dioxopiperidin-3-yl)-1-methyl-1H-indazol-6-yl)piperidin-4-yl)methyl)-2,7-diazaspiro[3.5]nonan-2-yl)-2-(trifluoromethoxy)benzenesulfonamide;
N-(6-((1H-Pyrazol-1-yl)methyl)-4-methoxybenzo[d]isoxazol-3-yl)-3-(7-((1-(3-(2,6-dioxopiperidin-3-yl)-1-methyl-1H-indazol-6-yl)piperidin-4-yl)methyl)-7-azaspiro[3.5]nonan-2-yl)benzenesulfonamide;
N-(6-((1H-Pyrazol-1-yl)methyl)-4-methoxybenzo[d]isoxazol-3-yl)-3-(2-((4-(3-(2,4-dioxotetrahydropyrimidin-1(2H)-yl)-1-methyl-1H-indazol-6-yl)piperidin-1-yl)methyl)-7-azaspiro[3.5]nonan-7-yl)benzenesulfonamide;
N-(6-((1H-Pyrazol-1-yl)methyl)-4-methoxybenzo[d]isoxazol-3-yl)-3-(2-((4-(3-(2,6-dioxopiperidin-3-yl)-1-methyl-1H-indazol-6-yl)piperidin-1-yl)methyl)-7-azaspiro[3.5]nonan-7-yl)benzenesulfonamide;
N-(6-((1H-Pyrazol-1-yl)methyl)-4-methoxybenzo[d]isoxazol-3-yl)-3-(2-((4-((1-(2,6-dioxopiperidin-3-yl)-3-methyl-2-oxo-2,3-dihydro-1H-benzo[d]imidazol-4-yl)ethynyl)piperidin-1-yl)methyl)-7-azaspiro[3.5]nonan-7-yl)benzenesulfonamide;
or a pharmaceutically acceptable salt thereof.

118. The compound according to claim 1 that is:
3-(7-((1-(3-(2,6-Dioxopiperidin-3-yl)-1-methyl-1H-indazol-6-yl)piperidin-4-yl)methyl)-2,7-diazaspiro[3.5]nonan-2-yl)-5-fluoro-2,6-dimethoxy-N-(4-methoxy-6-(methoxymethyl)-benzo[d]isoxazol-3-yl)benzenesulfonamide;
3-(7-((1-(3-(2,4-Dioxotetrahydropyrimidin-1(2H)-yl)-1-methyl-1H-indazol-6-yl)-piperidin-4-yl)methyl)-2,7-diazaspiro[3.5]nonan-2-yl)-5-fluoro-2,6-dimethoxy-N-(4-methoxy-6-(methoxymethyl)benzo[d]isoxazol-3-yl)benzenesulfonamide;
3-(7-((1-(3-(2,6-Dioxopiperidin-3-yl)-1-methyl-1H-pyrrolo[2,3-b]pyridin-6-yl)piperidin-4-yl)methyl)-2,7-diazaspiro[3.5]nonan-2-yl)-5-fluoro-2,6-dimethoxy-N-(4-methoxy-6-(methoxy-methyl)benzo[d]isoxazol-3-yl)benzenesulfonamide;
3-(2-((1-(3-(2,6-Dioxopiperidin-3-yl)-1-methyl-1H-indazol-6-yl)piperidin-4-yl)methyl)-1,2,3,4-tetrahydroisoquinolin-6-yl)-5-fluoro-2,6-dimethoxy-N-(4-methoxy-6-(methoxymethyl)-benzo[d]isoxazol-3-yl)benzenesulfonamide;
3-(2-((1-(3-(2,4-Dioxotetrahydropyrimidin-1(2H)-yl)-1-methyl-1H-indazol-6-yl)-piperidin-4-yl)methyl)-1,2,3,4-tetrahydroisoquinolin-6-yl)-5-fluoro-2,6-dimethoxy-N-(4-methoxy-6-(methoxymethyl)benzo[d]isoxazol-3-yl)benzenesulfonamide;
4′-((4-(3-(2,6-Dioxopiperidin-3-yl)-1-methyl-1H-indazol-6-yl)piperidin-1-yl)methyl)-5-fluoro-2,4-dimethoxy-N-(4-methoxy-6-(methoxymethyl)benzo[d]isoxazol-3-yl)-3′-methyl-[1,1′-biphenyl]-3-sulfonamide;
4′-((4-(3-(2,4-Dioxotetrahydropyrimidin-1(2H)-yl)-1-methyl-1H-indazol-6-yl)piperidin-1-yl)methyl)-5-fluoro-2,4-dimethoxy-N-(4-methoxy-6-(methoxymethyl)benzo-[d]isoxazol-3-yl)-3′-methyl-[1,1′-biphenyl]-3-sulfonamide;
N-(6-((1H-Pyrazol-1-yl)methyl)-4-methoxybenzo[d]isoxazol-3-yl)-3-(7-((1-(3-(2,4-dioxotetrahydropyrimidin-1(2H)-yl)-1-methyl-1H-indazol-6-yl)piperidin-4-yl)methyl)-2,7-diazaspiro[3.5]nonan-2-yl)-5-fluoro-2,6-dimethoxybenzenesulfonamide;
rac-3-((cis)-3-((1-(3-(2,4-Dioxotetrahydropyrimidin-1(2H)-yl)-1-methyl-1H-indazol-6-yl)piperidin-4-yl)methyl)-3,7-diazabicyclo[4.2.0]octan-7-yl)-5-fluoro-2,6-dimethoxy-N-(4-methoxy-6-(methoxymethyl)benzo[d]isoxazol-3-yl)benzenesulfonamide;
3-((1-(3-(2,6-Dioxopiperidin-3-yl)-1-methyl-1H-pyrrolo[2,3-b]pyridin-6-yl)piperidin-4-yl)methyl)-3,7-diazabicyclo[4.2.0]octan-7-yl)-5-fluoro-2,6-dimethoxy-N-(4-methoxy-6-(methoxymethyl)benzo[d]isoxazol-3-yl)benzenesulfonamide;
rac-3-((cis)-3-((1-(3-(2,4-Dioxotetrahydropyrimidin-1(2H)-yl)-1-methyl-1H-indazol-6-yl)piperidin-4-yl)methyl)-3,7-diazabicyclo[4.2.0]octan-7-yl)-2-methoxy-N-(4-methoxy-6-(methoxymethyl)benzo[d]isoxazol-3-yl)-5-(trifluoromethyl)benzenesulfonamide;
3-((1-(3-(2,6-Dioxopiperidin-3-yl)-1-methyl-1H-pyrrolo[2,3-b]pyridin-6-yl)piperidin-4-yl)methyl)-3,7-diazabicyclo[4.2.0]octan-7-yl)-2-methoxy-N-(4-methoxy-6-(methoxymethyl)-benzo[d]isoxazol-3-yl)-5-(trifluoromethyl)benzenesulfonamide;
N-(6-((1H-pyrazol-1-yl)methyl)-4-methoxybenzo[d]isoxazol-3-yl)-3-(6-((1-(3-(2,4-dioxotetrahydropyrimidin-1(2H)-yl)-1-methyl-1H-indazol-6-yl)piperidin-4-yl)methyl)-5,6,7,8-tetrahydro-1,6-naphthyridin-2-yl)-5-fluoro-2,6-dimethoxybenzene-sulfonamide;
N-(6-((1H-pyrazol-1-yl)methyl)-4-methoxybenzo[d]isoxazol-3-yl)-3-(7-((1-(4-(2,6-dioxopiperidin-3-yl)phenyl)piperidin-4-yl)methyl)-2,7-diazaspiro[3.5]nonan-2-yl)-5-fluoro-2,6-dimethoxybenzenesulfonamide;
rac-3-(cis)-N-(6-((1H-pyrazol-1-yl)methyl)-4-methoxybenzo[d]isoxazol-3-yl)-3-((1-(3-(2,4-dioxotetrahydropyrimidin-1(2H)-yl)-1-methyl-1H-indazol-6-yl)-piperidin-4-yl)methyl)-3,7-diazabicyclo[4.2.0]octan-7-yl)-5-fluoro-2,6-dimethoxy-benzenesulfonamide;
or a pharmaceutically acceptable salt thereof.

119. The compound according to claim 1 that is:
N-(6-((1H-Pyrazol-1-yl)methyl)-4-methoxybenzo[d]isoxazol-3-yl)-3-(7-((4-(3-(2,4-dioxotetrahydropyrimidin-1(2H)-yl)-1-methyl-1H-indazol-6-yl)piperazin-1-yl)methyl)-2-azaspiro[3.5]nonan-2-yl)benzenesulfonamide;
N-(6-((1H-Pyrazol-1-yl)methyl)-4-methoxybenzo[d]isoxazol-3-yl)-3-(7-((4-(4-(2,6-dioxopiperidin-3-yl)phenyl)piperidin-1-yl)methyl)-2-azaspiro[3.5]nonan-2-yl)-5-fluoro-2,6-dimethoxybenzenesulfonamide;
N-(6-((1H-Pyrazol-1-yl)methyl)-4-methoxybenzo[d]isoxazol-3-yl)-3-(7-((1-(3-(2,6-dioxopiperidin-3-yl)-1-methyl-1H-indazol-6-yl)piperidin-4-yl)methyl)-2,7-diazaspiro[3.5]nonan-2-yl)benzenesulfonamide;
N-(6-((1H-Pyrazol-1-yl)methyl)-4-methoxybenzo[d]isoxazol-3-yl)-3-(7-((1-(3-(2,4-dioxotetrahydropyrimidin-1(2H)-yl)-1-methyl-1H-pyrazolo[3,4-b]pyridin-6-yl)piperidin-4-yl)methyl)-2,7-diazaspiro[3.5]nonan-2-yl)-2,6-dimethoxybenzenesulfonamide;
N-(6-((1H-Pyrazol-1-yl)methyl)-4-methoxybenzo[d]isoxazol-3-yl)-3-(7-((1-(3-(2,6-dioxopiperidin-3-yl)-1-methyl-1H-pyrazolo[3,4-b]pyridin-6-yl)piperidin-4-yl)methyl)-2,7-diazaspiro[3.5]nonan-2-yl)-5-fluoro-2,6-dimethoxybenzenesulfonamide;
N-(6-((1H-Pyrazol-1-yl)methyl)-4-methoxybenzo[d]isoxazol-3-yl)-3-(7-((1-(3-(2,4-dioxotetrahydropyrimidin-1(2H)-yl)-7-fluoro-1-methyl-1H-indazol-6-yl)piperidin-4-yl)methyl)-2,7-diazaspiro[3.5]nonan-2-yl)-5-fluoro-2,6-dimethoxybenzenesulfonamide;
N-(6-((1H-Pyrazol-1-yl)methyl)-4-methoxybenzo[d]isoxazol-3-yl)-3-(4-((7-(3-(2,4-dioxotetrahydropyrimidin-1(2H)-yl)-1-methyl-1H-indazol-6-yl)-7-azaspiro[3.5]nonan-2-yl)methyl)piperazin-1-yl)-5-fluoro-2,6-dimethoxybenzenesulfonamide;
N-(6-((1H-Pyrazol-1-yl)methyl)-4-methoxybenzo[d]isoxazol-3-yl)-3-(6-((1-(3-(2,6-dioxopiperidin-3-yl)-1-methyl-1H-indazol-6-yl)piperidin-4-yl)methyl)-5,6,7,8-tetrahydro-1,6-naphthyridin-2-yl)-5-fluoro-2,6-dimethoxybenzenesulfonamide;
N-(6-((1H-Pyrazol-1-yl)methyl)-4-methoxybenzo[d]isoxazol-3-yl)-4′-((4-(3-(2,4-dioxotetrahydropyrimidin-1(2H)-yl)-1-methyl-1H-indazol-6-yl)piperidin-1-yl)methyl)-2,4-dimethoxy-3′-methyl-[1,1′-biphenyl]-3-sulfonamide;
N-(6-((1H-Pyrazol-1-yl)methyl)-4-methoxybenzo[d]isoxazol-3-yl)-4′-((4-(3-(2,4-dioxotetrahydropyrimidin-1(2H)-yl)-1-methyl-1H-indazol-6-yl)piperidin-1-yl)methyl)-2,2′,4-trimethoxy-[1,1′-biphenyl]-3-sulfonamide;
N-(6-((1H-Pyrazol-1-yl)methyl)-4-methoxybenzo[d]isoxazol-3-yl)-4′-((4-(3-(2,6-dioxopiperidin-3-yl)-1-methyl-1H-pyrrolo[2,3-b]pyridin-6-yl)piperidin-1-yl)methyl)-2,3′,4-trimethoxy-[1,1′-biphenyl]-3-sulfonamide;
N-(6-((1H-Pyrazol-1-yl)methyl)-4-methoxybenzo[d]isoxazol-3-yl)-4′-((4-(3-(2,6-dioxopiperidin-3-yl)-1-methyl-1H-indazol-6-yl)piperidin-1-yl)methyl)-2,4-dimethoxy-3′-(trifluoromethoxy)-[1,1′-biphenyl]-3-sulfonamide;
N-(6-((1H-Pyrazol-1-yl)methyl)-4-methoxybenzo[d]isoxazol-3-yl)-4′-((4-(3-(2,4-dioxotetrahydropyrimidin-1(2H)-yl)-1-methyl-1H-indazol-6-yl)piperidin-1-yl)methyl)-2′-fluoro-2,4-dimethoxy-[1,1′-biphenyl]-3-sulfonamide;
N-(6-((1H-Pyrazol-1-yl)methyl)-4-methoxybenzo[d]isoxazol-3-yl)-4′-((4-(3-(2,6-dioxopiperidin-3-yl)-1-methyl-1H-indazol-6-yl)piperidin-1-yl)methyl)-3′-fluoro-2,4-dimethoxy-[1,1′-biphenyl]-3-sulfonamide;
N-(6-((1H-Pyrazol-1-yl)methyl)-4-methoxybenzo[d]isoxazol-3-yl)-4′-((4-(3-(2,6-dioxopiperidin-3-yl)-1-methyl-1H-indazol-6-yl)piperidin-1-yl)methyl)-2′,5′-difluoro-2,4-dimethoxy-[1,1′-biphenyl]-3-sulfonamide;
N-(6-((1H-Pyrazol-1-yl)methyl)-4-methoxybenzo[d]isoxazol-3-yl)-4′-((4-(3-(2,4-dioxotetrahydropyrimidin-1(2H)-yl)-1-methyl-1H-indazol-6-yl)piperidin-1-yl)methyl)-2,4-dimethoxy-3′-(trifluoromethyl)-[1,1′-biphenyl]-3-sulfonamide;
N-(6-((1H-Pyrazol-1-yl)methyl)-4-methoxybenzo[d]isoxazol-3-yl)-4′-((4-(3-(2,6-dioxopiperidin-3-yl)-1-methyl-1H-indazol-6-yl)piperidin-1-yl)methyl)-2,4-dimethoxy-2′-(trifluoromethyl)-[1,1′-biphenyl]-3-sulfonamide;
N-(6-((1H-Pyrazol-1-yl)methyl)-4-methoxybenzo[d]isoxazol-3-yl)-2′-cyano-4′-((4-(3-(2,4-dioxotetrahydropyrimidin-1(2H)-yl)-1-methyl-1H-indazol-6-yl)piperidin-1-yl)methyl)-2,4-dimethoxy-[1,1′-biphenyl]-3-sulfonamide;
N-(6-((1H-Pyrazol-1-yl)methyl)-4-methoxybenzo[d]isoxazol-3-yl)-3-(5-((4-(3-(2,6-dioxopiperidin-3-yl)-1-methyl-1H-indazol-6-yl)piperidin-1-yl)methyl)thiophen-3-yl)-2,6-dimethoxybenzenesulfonamide;
N-(6-((1H-Pyrazol-1-yl)methyl)-4-methoxybenzo[d]isoxazol-3-yl)-3-(2-((1-(3-(2,6-dioxopiperidin-3-yl)-1-methyl-1H-indazol-6-yl)piperidin-4-yl)methyl)-1,2,3,4-tetrahydro-isoquinolin-6-yl)-5-fluoro-2,6-dimethoxybenzenesulfonamide;
N-(6-((1H-Pyrazol-1-yl)methyl)-4-methoxybenzo[d]isoxazol-3-yl)-3-(2-((1-(3-(2,4-dioxotetrahydropyrimidin-1(2H)-yl)-1-methyl-1H-indazol-6-yl)piperidin-4-yl)methyl)-isoindolin-5-yl)-5-fluoro-2,6-dimethoxybenzenesulfonamide;
N-(6-((1H-Pyrazol-1-yl)methyl)-4-methoxybenzo[d]isoxazol-3-yl)-3-(2-((1-(3-(2,4-dioxotetrahydropyrimidin-1(2H)-yl)-1-methyl-1H-indazol-6-yl)piperidin-4-yl)methyl)-1,2,3,4-tetrahydroisoquinolin-6-yl)-6-fluoro-2-methoxybenzenesulfonamide;
N-(6-((1H-Pyrazol-1-yl)methyl)-4-methoxybenzo[d]isoxazol-3-yl)-3-(2-((1-(3-(2,4-dioxotetrahydropyrimidin-1(2H)-yl)-1-methyl-1H-indazol-6-yl)piperidin-4-yl)methyl)-1,2,3,4-tetrahydroisoquinolin-7-yl)-2-methoxy-5-(trifluoromethyl)benzenesulfonamide;
N-(6-((1H-Pyrazol-1-yl)methyl)-4-methoxybenzo[d]isoxazol-3-yl)-5-(tert-butyl)-3-(6-((1-(3-(2,4-dioxotetrahydropyrimidin-1(2H)-yl)-1-methyl-1H-indazol-6-yl)piperidin-4-yl)methyl)-5,6,7,8-tetrahydro-1,6-naphthyridin-2-yl)-2-methoxybenzenesulfonamide;
N-(6-((1H-Pyrazol-1-yl)methyl)-4-methoxybenzo[d]isoxazol-3-yl)-3-(2-((4-(3-(2,6-dioxopiperidin-3-yl)-1-methyl-1H-indazol-6-yl)piperidin-1-yl)methyl)pyrimidin-5-yl)-2,6-dimethoxybenzenesulfonamide;
N-(6-((1H-Pyrazol-1-yl)methyl)-4-methoxybenzo[d]isoxazol-3-yl)-3-(6-((4-(3-(2,6-dioxopiperidin-3-yl)-1-methyl-1H-indazol-6-yl)piperidin-1-yl)methyl)-2-methylpyridin-3-yl)-5-fluoro-2,6-dimethoxybenzenesulfonamide;
N-(6-((1H-Pyrazol-1-yl)methyl)-4-methoxybenzo[d]isoxazol-3-yl)-4′-(4-(3-(2,6-dioxopiperidin-3-yl)-1-methyl-1H-indazol-6-yl)piperidine-1-carbonyl)-2,4-dimethoxy-[1,1′-biphenyl]-3-sulfonamide;
N-(6-((1H-Pyrazol-1-yl)methyl)-4-methoxybenzo[d]isoxazol-3-yl)-3-(1-((1-(3-(2,4-dioxotetrahydropyrimidin-1(2H)-yl)-1-methyl-1H-indazol-6-yl)piperidin-4-yl)methyl)-piperidin-4-yl)-5-fluoro-2,6-dimethoxybenzenesulfonamide;
N-(6-((1H-Pyrazol-1-yl)methyl)-4-methoxybenzo[d]isoxazol-3-yl)-3-(4-((4-(3-(2,4-dioxotetrahydropyrimidin-1(2H)-yl)-1-methyl-1H-indazol-6-yl)piperidin-1-yl)methyl)cyclo-hexyl)-5-fluoro-2,6-dimethoxybenzenesulfonamide;
N-(6-((1H-Pyrazol-1-yl)methyl)-4-methoxybenzo[d]isoxazol-3-yl)-3-(3-((1-(3-(2,6-dioxopiperidin-3-yl)-1-methyl-1H-indazol-6-yl)piperidin-4-yl)methyl)-3-azaspiro[5.5]-undecan-9-yl)-2,6-dimethoxybenzenesulfonamide;
N-(6-((1H-Pyrazol-1-yl)methyl)-4-methoxybenzo[d]isoxazol-3-yl)-3-((1-((1-(3-(2,6-dioxopiperidin-3-yl)-1-methyl-1H-pyrrolo[2,3-b]pyridin-6-yl)piperidin-4-yl)methyl)piperidin-4-yl)methyl)-5-fluoro-2,6-dimethoxybenzenesulfonamide;
N-(6-((1H-Pyrazol-1-yl)methyl)-4-methoxybenzo[d]isoxazol-3-yl)-4′-((4-(3-(2,4-dioxotetrahydropyrimidin-1(2H)-yl)-1-methyl-1H-indazol-6-yl)piperidin-1-yl)methyl)-2,4-dimethoxy-[1,1′-biphenyl]-3-sulfonamide;
N-(6-((1H-Pyrazol-1-yl)methyl)-4-methoxybenzo[d]isoxazol-3-yl)-4′-((4-(3-(2,4-dioxotetrahydropyrimidin-1(2H)-yl)-1-methyl-1H-indazol-6-yl)piperazin-1-yl)methyl)-5-fluoro-2,4-dimethoxy-2′-(trifluoromethyl)-[1,1′-biphenyl]-3-sulfonamide;
N-(6-((1H-Pyrazol-1-yl)methyl)-4-methoxybenzo[d]isoxazol-3-yl)-4′-((3-(3-(2,6-dioxopiperidin-3-yl)-1-methyl-1H-indazol-6-yl)pyrrolidin-1-yl)methyl)-5-fluoro-2,4-dimethoxy-2′-(trifluoromethyl)-[1,1′-biphenyl]-3-sulfonamide;
rac-N-(6-((1H-Pyrazol-1-yl)methyl)-4-methoxybenzo[d]isoxazol-3-yl)-3-((3a,7a)-5-((1-(3-(2,4-dioxotetrahydropyrimidin-1(2H)-yl)-1-methyl-1H-indazol-6-yl)piperidin-4-yl)methyl)octahydro-2H-pyrrolo[3,4-c]pyridin-2-yl)-5-fluoro-2,6-dimethoxy-benzenesulfonamide;
N-(6-((1H-Pyrazol-1-yl)methyl)-4-methoxybenzo[d]isoxazol-3-yl)-3-((4aR,8aR)-6-((1-(3-(2,4-dioxotetrahydropyrimidin-1(2H)-yl)-1-methyl-1H-indazol-6-yl)piperidin-4-yl)methyl)octahydro-2,6-naphthyridin-2(1H)-yl)-5-fluoro-2,6-dimethoxybenzenesulfonamide;
N-(6-((1H-Pyrazol-1-yl)methyl)-4-methoxybenzo[d]isoxazol-3-yl)-3-((3aR,6aR)-5-((1-(3-(2,4-dioxotetrahydropyrimidin-1(2H)-yl)-1-ethyl-1H-indazol-6-yl)piperidin-4-yl)methyl)hexahydropyrrolo[3,4-c]pyrrol-2(1H)-yl)-2,6-dimethoxybenzenesulfonamide;
N-(6-((1H-Pyrazol-1-yl)methyl)-4-methoxybenzo[d]isoxazol-3-yl)-3-(11-((1-(3-(2,4-dioxotetrahydropyrimidin-1(2H)-yl)-1-methyl-1H-indazol-6-yl)piperidin-4-yl)methyl)-3,11-diazadispiro[5.1.58.16]tetradecan-3-yl)-2,6-dimethoxybenzenesulfonamide;
N-(6-((1H-Pyrazol-1-yl)methyl)-4-methoxybenzo[d]isoxazol-3-yl)-3-(7-((1-(3-(2,4-dioxotetrahydropyrimidin-1(2H)-yl)-1-ethyl-1H-indazol-6-yl)piperidin-4-yl)methyl)-2,7-diazaspiro[3.5]nonan-2-yl)-5-fluoro-2,6-dimethoxybenzenesulfonamide;
N-(6-((1H-Pyrazol-1-yl)methyl)-4-methoxybenzo[d]isoxazol-3-yl)-3-(7-((1-(4-(2,6-dioxopiperidin-3-yl)-2-methylphenyl)piperidin-4-yl)methyl)-2,7-diazaspiro[3.5]nonan-2-yl)-5-fluoro-2,6-dimethoxybenzenesulfonamide;
N-(6-((1H-Pyrazol-1-yl)methyl)-4-methoxybenzo[d]isoxazol-3-yl)-3-(7-((1-(4-(2,6-dioxopiperidin-3-yl)-3-fluorophenyl)piperidin-4-yl)methyl)-2,7-diazaspiro[3.5]nonan-2-yl)-2-methoxybenzenesulfonamide;
N-(6-((1H-Pyrazol-1-yl)methyl)-4-methoxybenzo[d]isoxazol-3-yl)-3-(7-((1-(4-(2,6-dioxopiperidin-3-yl)-3,5-difluorophenyl)piperidin-4-yl)methyl)-2,7-diazaspiro[3.5]nonan-2-yl)-2-methoxybenzenesulfonamide;
N-(6-((1H-Pyrazol-1-yl)methyl)-4-methoxybenzo[d]isoxazol-3-yl)-3-(7-(2-(1-(4-(2,6-dioxopiperidin-3-yl)phenyl)piperidin-4-yl)ethyl)-2,7-diazaspiro[3.5]nonan-2-yl)-5-fluoro-2,6-dimethoxybenzenesulfonamide;
N-(6-((1H-Pyrazol-1-yl)methyl)-4-methoxybenzo[d]isoxazol-3-yl)-3-(7-(((3S)-1-(1-(2,6-dioxopiperidin-3-yl)-3-methyl-1H-indazol-5-yl)pyrrolidin-3-yl)methyl)-2,7-diazaspiro-[3.5]nonan-2-yl)-5-fluoro-2,6-dimethoxybenzenesulfonamide;
N-(6-((1H-Pyrazol-1-yl)methyl)-4-methoxybenzo[d]isoxazol-3-yl)-3-(7-(((3S)-1-(4-(2,6-dioxopiperidin-3-yl)phenyl)pyrrolidin-3-yl)methyl)-2,7-diazaspiro[3.5]nonan-2-yl)-5-fluoro-2,6-dimethoxybenzenesulfonamide;
N-(6-((1H-Pyrazol-1-yl)methyl)-4-methoxybenzo[d]isoxazol-3-yl)-3-(7-(((3R,4S)-1-(4-(2,6-dioxopiperidin-3-yl)phenyl)-3-fluoropiperidin-4-yl)methyl)-2,7-diazaspiro[3.5]nonan-2-yl)-5-fluoro-2,6-dimethoxybenzenesulfonamide;
N-(6-((1H-Pyrazol-1-yl)methyl)-4-methoxybenzo[d]isoxazol-3-yl)-3-(7-((1-(3-(2,4-dioxotetrahydropyrimidin-1(2H)-yl)-1-methyl-1H-pyrrolo[2,3-b]pyridin-6-yl)-4-fluoro-piperidin-4-yl)methyl)-2,7-diazaspiro[3.5]nonan-2-yl)-2-methoxybenzenesulfonamide;
3-[7-[[4-[4-(2,6-Dioxopiperidin-3-yl)phenyl]piperazin-1-yl]methyl]-2-azaspiro-[3.5]nonan-2-yl]-5-fluoro-2-methoxy-N-[4-methoxy-6-(pyrazol-1-ylmethyl)-1,2-benzoxazol-3-yl]benzenesulfonamide;
N-(6-((1H-Pyrazol-1-yl)methyl)-4-methoxybenzo[d]isoxazol-3-yl)-3-((1S,6S)-3-((1-(3-(2,4-dioxotetrahydropyrimidin-1(2H)-yl)-1-methyl-1H-indazol-6-yl)piperidin-4-yl)methyl)-3,7-diazabicyclo[4.2.0]octan-7-yl)-2-methoxy-6-methylbenzenesulfonamide;
N-(6-((1H-Pyrazol-1-yl)methyl)-4-methoxybenzo[d]isoxazol-3-yl)-3-(7-((1-(3-(2,6-dioxopiperidin-3-yl)-5-fluoro-1-methyl-1H-indazol-6-yl)piperidin-4-yl)methyl)-2,7-diazaspiro[3.5]nonan-2-yl)-5-fluoro-2,6-dimethoxybenzenesulfonamide;
N-(6-((1H-Pyrazol-1-yl)methyl)-4-methoxybenzo[d]isoxazol-3-yl)-3-((3R)-3-((4-(3-(2,6-dioxopiperidin-3-yl)-1-methyl-1H-indazol-6-yl)piperidin-1-yl)methyl)piperidin-1-yl)-2,6-dimethoxybenzenesulfonamide;
3-[6-[[4-[3-(2,6-Dioxopiperidin-3-yl)-1-methylindazol-6-yl]piperidin-1-yl]methyl]-2-pyridinyl]-2,6-dimethoxy-N-[4-methoxy-6-(pyrazol-1-ylmethyl)-1,2-benzoxazol-3-yl]benzenesulfonamide;
N-(6-((1H-Pyrazol-1-yl)methyl)-4-methoxybenzo[d]isoxazol-3-yl)-3-(4-((4-(3-(2,6-dioxopiperidin-3-yl)-1-methyl-1H-indazol-6-yl)piperidin-1-yl)methyl)pyridin-2-yl)-2,6-dimethoxybenzenesulfonamide;
N-(6-((1H-Pyrazol-1-yl)methyl)-4-methoxybenzo[d]isoxazol-3-yl)-3-(8-((1-(3-(2,4-dioxotetrahydropyrimidin-1(2H)-yl)-1-methyl-1H-indazol-6-yl)piperidin-4-yl)methyl)-8-azabicyclo[3.2.1]octan-3-yl)-5-fluoro-2,6-dimethoxybenzenesulfonamide;
N-(6-((1H-Pyrazol-1-yl)methyl)-4-methoxybenzo[d]isoxazol-3-yl)-3-(7-((1-(2-(2,6-dioxopiperidin-3-yl)-1-methyl-1H-pyrrolo[2,3-b]pyridin-6-yl)piperidin-4-yl)methyl)-2,7-diazaspiro[3.5]nonan-2-yl)-2-methoxybenzenesulfonamide;
N-(6-((1H-Pyrazol-1-yl)methyl)-4-methoxybenzo[d]isoxazol-3-yl)-3-(7-((1-(2-(2,6-dioxopiperidin-3-yl)-1-methyl-1H-pyrrolo[2,3-b]pyridin-6-yl)piperidin-4-yl)methyl)-2,7-diazaspiro[3.5]nonan-2-yl)-5-fluoro-2,6-dimethoxybenzenesulfonamide;
N-(6-((1H-Pyrazol-1-yl)methyl)-4-methoxybenzo[d]isoxazol-3-yl)-3-(7-((1-(5-(2,6-dioxopiperidin-3-yl)pyridin-2-yl)piperidin-4-yl)methyl)-2,7-diazaspiro[3.5]nonan-2-yl)-5-fluoro-2,6-dimethoxybenzenesulfonamide;
4-(4-((2-(3-(N-(6-((1H-Pyrazol-1-yl)methyl)-4-methoxybenzo[d]isoxazol-3-yl)sulfamoyl)-5-fluoro-2,4-dimethoxyphenyl)-2,7-diazaspiro[3.5]nonan-7-yl)methyl)-piperidin-1-yl)-N-(2,6-dioxopiperidin-3-yl)benzamide;
(R)—N-(6-((1H-Pyrazol-1-yl)methyl)-4-methoxybenzo[d]isoxazol-3-yl)-3-(7-((1-(4-(2,6-dioxopiperidin-3-yl-3,4,4,5,5-d5)phenyl)piperidin-4-yl)methyl)-2,7-diazaspiro[3.5]-nonan-2-yl)-5-fluoro-2,6-dimethoxybenzenesulfonamide;
(R)—N-(6-((1H-Pyrazol-1-yl)methyl)-4-methoxybenzo[d]isoxazol-3-yl)-3-(7-((1-(4-(2,6-dioxopiperidin-3-yl)phenyl)piperidin-4-yl)methyl)-2,7-diazaspiro[3.5]nonan-2-yl)-5-fluoro-2,6-dimethoxybenzenesulfonamide;
(S)—N-(6-((1H-Pyrazol-1-yl)methyl)-4-methoxybenzo[d]isoxazol-3-yl)-3-(7-((1-(4-(2,6-dioxopiperidin-3-yl)phenyl)piperidin-4-yl)methyl)-2,7-diazaspiro[3.5]nonan-2-yl)-5-fluoro-2,6-dimethoxybenzenesulfonamide;
N-(6-((1H-Pyrazol-1-yl)methyl)-4-methoxybenzo[d]isoxazol-3-yl)-3-(6-((1-(3-(2,6-dioxopiperidin-3-yl)-1-(methyl-d3)-1H-pyrrolo[2,3-b]pyridin-6-yl)piperidin-4-yl)methyl)-5,6,7,8-tetrahydro-1,6-naphthyridin-2-yl)-5-fluoro-2,6-dimethoxybenzene-sulfonamide;
N-(6-((1H-Pyrazol-1-yl)methyl)-4-methoxybenzo[d]isoxazol-3-yl)-3-(6-((1-(3-(2,4-dioxotetrahydropyrimidin-1(2H)-yl)-1-(methyl-d3)-1H-indazol-6-yl)piperidin-4-yl)methyl)-5,6,7,8-tetrahydro-1,6-naphthyridin-2-yl)-5-fluoro-2,6-dimethoxybenzenesulfonamide;
N-(6-((1H-Pyrazol-1-yl)methyl)-4-methoxybenzo[d]isoxazol-3-yl)-3-(7-((1-(3-(2,6-dioxopiperidin-3-yl)-1-(2,2,2-trifluoroethyl)-1H-indazol-6-yl)piperidin-4-yl)methyl)-2,7-diazaspiro[3.5]nonan-2-yl)-5-fluoro-2,6-dimethoxybenzenesulfonamide;
N-(6-((1H-Pyrazol-1-yl)methyl)-4-methoxybenzo[d]isoxazol-3-yl)-3-(2-((1-(3-(2,6-dioxopiperidin-3-yl)-1-methyl-1H-indazol-6-yl)piperidin-4-yl)methyl)-2,8-diazaspiro[4.5]decan-8-yl)-5-fluoro-2,6-dimethoxybenzenesulfonamide;
N-(6-((1H-Pyrazol-1-yl)methyl)-4-methoxybenzo[d]isoxazol-3-yl)-3-(7-((1-(3-(2,6-dioxopiperidin-3-yl)-1-methyl-1H-indazol-6-yl)piperidin-4-yl)methyl)-5,6,7,8-tetrahydro-imidazo[1,2-a]pyrazin-3-yl)-2,6-dimethoxybenzenesulfonamide;
N-(6-((1H-Pyrazol-1-yl)methyl)-4-methoxybenzo[d]isoxazol-3-yl)-3-(7-(3-(2,6-dioxo-piperidin-3-yl)-1-methyl-1H-indazol-6-yl)-2,7-diazaspiro[3.5]nonan-2-yl)-2-methoxybenzene-sulfonamide;
or a pharmaceutically acceptable salt thereof.

120. The compound of claim 1, in the form of a pharmaceutically acceptable salt.

121. A pharmaceutical composition comprising a compound according to claim 1, or a pharmaceutically acceptable salt thereof, and a pharmaceutically acceptable excipient.

122. A method of treating cancer or solid tumors in a subject in need thereof comprising administering to the subject a compound of claim 1 or a pharmaceutical composition comprising the compound.

123. The method of claim 122, wherein the cancer is selected from the group consisting of lung cancer, mesothelioma, bone cancer, pancreatic cancer, skin cancer, cancer of the head or neck, cutaneous or intraocular melanoma, uterine cancer, ovarian cancer, rectal cancer, cancer of the anal region, stomach cancer, hepatic carcinoma, colon cancer, breast cancer, uterine cancer, carcinoma of the fallopian tubes, carcinoma of the endometrium, carcinoma of the cervix, carcinoma of the vagina, carcinoma of the vulva, Hodgkin's disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, hematology malignancy, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, glioblastoma, brain stem glioma, pituitary adenoma, and a combination of two or more of the foregoing cancers.

124. The method of claim 122, wherein the solid tumors are selected from the group consisting of breast, lung, colon, brain, prostate, stomach, pancreatic, ovarian, melanoma, endocrine, uterine, testicular, hematologic and bladder.

125.-135. (canceled)

136. A method of degrading a KAT6 protein comprising contacting the KAT6 protein with a compound of claim 1 or a pharmaceutical composition comprising the compound.
